(19) United States
(12) Patent Application Publication
(10) Pub. No.: US 2024/0228570 A9
(43) Pub. Date: Jul. 11, 2024

(54) PLATFORM TECHNOLOGY FOR TREATMENT OF INFLAMMATORY, IMMUNOLOGICAL AND/OR AUTOIMMUNOLOGICAL DISEASES

(71) Applicant: ellennbe GmbH, Bad Heilbrunn (DE)

(72) Inventor: Lydia Ellen Neumann, Bad Heilbrunn (DE)

(21) Appl. No.: 18/491,113

(22) Filed: Oct. 20, 2023

Related U.S. Application Data
(63) Continuation-in-part of application No. PCT/EP2022/079665, filed on Oct. 24, 2022.
(60) Provisional application No. 63/380,647, filed on Oct. 24, 2022.

(30) Foreign Application Priority Data
Oct. 25, 2021 (EP) 21204596.7

(51) Int. Cl. C07K 14/57; A61K 9/00; A61K 38/00; A61K 45/06; A61M 37/00; A61P 37/06; C07K 14/475; C07K 14/54; C07K 14/55

(52) U.S. Cl. CPC C07K 14/57; A61K 9/0019; A61K 45/06; A61M 37/0092; A61P 37/06; C07K 14/475; C07K 14/5406; C07K 14/55; A61K 38/00

(57) ABSTRACT

The invention relates to an immunomodulatory substance(s) and/or a skin-conditioning agent for use in a method for treating and/or preventing an inflammatory disease, immunological disease and/or autoimmunological disease in a subject, wherein the method comprises a step (A) selected from one or more of: generating an accumulation of PBMCs within the skin, generating a vasodilation of the capillaries within the skin, generating an increased blood volume within the skin, generating an increased sO₂ within the skin, generating an increased rHb within the skin, generating an increased temperature on the skin, generating a redness on the skin, administering conditioning energy to the skin, administering a skin-conditioning agent to the skin or administering PBMCs to the skin of the subject; and the method further comprises step (B) administering a first immunomodulatory substance(s) to the skin of the subject and/or step (C) administering a second immunomodulatory substance(s) to the skin of the subject.

### CROSS-REFERENCE TO RELATED APPLICATIONS

This application is a continuation-in-part of PCT Application No. PCT/EP2022/079665, filed on Oct. 24, 2022, which claims priority to EP Application No. 21204596.7, filed Oct. 25, 2021. This application also claims priority to U.S. Provisional Patent Application Ser. No. 63/380,647, filed on Oct. 24, 2022, and to U.S. Provisional Patent Application Ser. No. 63/380,300, filed on Oct. 20, 2022. These applications are hereby incorporated by reference in their entireties.

### INCORPORATION BY REFERENCE STATEMENT

This application incorporates by reference a sequence listing submitted herewith as Sequencelisting1944024.XML, created on Oct. 20, 2023, and having a file size of 6,806 bytes.

### BACKGROUND

The present invention relates to immunomodulatory substance(s) and/or a skin-conditioning agent for use in a method for treating and/or preventing diseases in a subject, such as an inflammatory disease, immunological disease and/or autoimmunological disease.

The invention further relates to pharmaceutical compositions, topical dosage forms, injectable dosage forms and kits of parts comprising an immunomodulatory substance(s) and/or skin-conditioning agent which may be used in the method for treating and/or preventing of the diseases in a subject. Moreover, the present invention relates to medical devices and kits of parts which may be used in the method for treating and/or preventing of the disease in a subject.

Inflammatory diseases, immunological diseases and/or autoimmunological diseases that cause damaging and painful inflammatory reactions with severe impact on life quality are widespread diseases which affect significant parts of the human and animal population. In particular, a chronic course of such diseases can cause extreme suffering over long periods of time for the concerned subjects and may frequently be life-shortening.

The methods and substances known for treatment and prevention of such diseases are still unsatisfactory to a large degree. For example, glucocorticoids are used for the treatment of these diseases, however, besides their beneficial effects in reducing inflammation, they have considerable negative side effects, especially in long-term use. In case of using for instance biologics and biosimilars like monoclonal antibodies the methods are additionally highly expensive, poorly tolerated by many patients, do not work in a significant proportion of patients and often lose their effect after a certain time.

On the other hand, immunotherapies may be available which, however, require immense personal and material resources and can only be provided in specialized health care centers. Hence, they are associated with a journey of the patients to the health care centers or are not accessible to them at all. Hence, such therapies are unsuitable for a broad public application, are time-consuming and cannot be sold as an off-the shelf therapy.

It is therefore highly desirable to provide alternative agents, articles and methods for the treatment and/or prevention of inflammatory diseases, immunological diseases and/or autoimmunological diseases, which, accordingly, is the object of the present invention.

### BRIEF SUMMARY

Highly surprisingly, it was found that this object can be solved by use of an immunomodulatory substance(s) in a method where it is brought in close vicinity and/or contact of Langerhans cells (LHs), peripheral regulatory T cells (peripheral Tregs) and/or peripheral blood mononuclear cells (PBMCs) and incubated in vivo in a subject's body part such as the skin.

The present invention is based on entirely new principles found for the treatment and/or prevention of an inflammatory disease, immunological disease and/or autoimmunological disease, and has a multitude of advantages for the subject's treated. Hence, the present invention provides a novel platform technology generally applicable for the treatment of inflammatory, immunological and/or autoimmunological diseases.

### BRIEF DESCRIPTION OF THE FIGURES AND DRAWINGS

The accompanying drawings, which are included to provide a further understanding of the invention and are incorporated in and constitute a part of this application, illustrate embodiments and effects of the invention and together with the description serve to explain the principle of the invention.

FIG. 1 shows a top view of the positions of the orientation areas O1 to O4 and the corresponding measurement areas M1 to M4 on the forearms of the subject tested including a scale on the table. The orientation and measurement areas were used for the O2C-measuments, the measurements of temperature on the skin and the redness of the skin.

FIG. 2A is a diagram showing the oxygen saturation of the hemoglobin (sO₂) within the skin measured over time, obtained from O2C-measurement, after topical application of a heat pad (M4) and without (M1 to M3). The table indicates the following values a), b) and c) at T(bp)_{base} before applying the heat pad, and at points in time T(bp)₁ to T(bp)₁₂ after the heat pad had been applied: a) values of the sO₂ in percent (%) obtained by O2C-measurement; b) calculated change of the sO₂ in %-points with reference to the value obtained at T(bp)_{base}; and c) elapsed time for T(bp)₁ to T(bp)₁₂ in seconds after removing the heat pad from M4.

FIG. 2B is a diagram showing the relative hemoglobin amount (rHb) within the skin measured over time, obtained from O2C-measurement, after topical application of a heat pad (M4) and without (M1 to M3). The table indicates the following values a), b) and c) at T(bp)_{base} before applying the heat pad, and at points in time T(bp)₁ to T(bp)₁₂ after the heat pad had been applied: a) values of the rHb in arbitrary units (AU) obtained by O2C-measurement; b) calculated change of the rHb in AU with reference to the value obtained at T(bp)_{base}; and c) elapsed time for T(bp)₁ to T(bp)₁₂ in seconds after removing the heat pad from M4.

FIG. 2C is a diagram showing the relative blood flow (rFlow) within the skin measured over time, obtained from O2C-measurement, after topical application of a heat pad (M4) and without (M1 to M3). The table indicates the following values a), b) and c) at T(bp)_{base} before applying the heat pad, and at points in time T(bp)₁ to T(bp)₁₂ after the heat pad had been applied: a) values of the rFlow in arbitrary units (AU) obtained by O2C-measurement; b) calculated change of the rFlow in AU with reference to the value obtained at T(bp)_{base}; and c) elapsed time for T(bp)₁ to T(bp)₁₂ in seconds after removing the heat pad from M4.

FIG. 2D is a diagram showing the blood flow velocity (Vel) within the skin measured over time, obtained from O2C-measurement, after topical application of a heat pad (M4) and without (M1 to M3). The table indicates the following values a), b) and c) at T(bp)_{base} before applying the heat pad, and at points in time T(bp)₁ to T(bp)₁₂ after the heat pad had been applied: a) values of the Vel in arbitrary units (AU) obtained by O2C-measurement; b) calculated change of the Vel in AU with reference to the value obtained at T(bp)_{base}; and c) elapsed time for T(bp)₁ to T(bp)₁₂ in seconds after removing the heat pad from M4.

FIG. 3A is a diagram showing the oxygen saturation of the hemoglobin (sO₂) within the skin measured over time, obtained from O2C-measurement, when topically applying a heat crème (M1), a comparative compound (M4) and without treatment (M2 and M3). The table indicates the following values a), b) and c) at T(bc)_{base} before applying the heat crème, and at points in time T(bc)₁ to T(bc)₁₃ after the heat crème was applied: a) values of the sO₂ in percent (%) obtained by O2C-measurement; b) calculated change of the sO₂ in 35%-points with reference to the value obtained at T(bc)_{base}; and c) elapsed time for T(bc)₁ to T(bc)₁₃ in seconds after finishing the application of the heat crème to M1.

FIG. 3B is a diagram showing the relative hemoglobin amount (rHb) within the skin measured over time, obtained from O2C-measurement, when topically applying a heat cream (M1), a comparative compound (M4) and without treatment (M2 and M3). The table indicates the following values a), b) and c) at T(bc)_{base} before applying the heat crème, and at points in time T(bc)₁ to T(bc)₁₃ after the heat crème was applied: a) values of the rHb in arbitrary units (AU) obtained by O2C-measurement; b) calculated change of the rHb in AU with reference to the value obtained at T(bc)_{base}; and c) elapsed time for T(bc)₁ to T(bc)₁₃ in seconds after finishing the application of the heat crème to M1.

FIG. 3C is a diagram showing the relative blood flow (rFlow) within the skin measured over time, obtained from O2C-measurement, when topically applying a heat cream (M1), a comparative compound (M4) and without treatment (M2 and M3). The table indicates the following values a), b) and c) at T(bc)_{base} before applying the heat crème, and at points in time T(bc)₁ to T(bc)₁₃ after the heat crème was applied: a) values of the rFlow in arbitrary units (AU) obtained by O2C-measurement; b) calculated change of the rFlow in AU with reference to the value obtained at T(bc)_{base}; and c) elapsed time for T(bc)₁ to T(bc)₁₃ in seconds after finishing the application of the heat crème to M1.

FIG. 3D is a diagram showing the blood flow velocity (Vel) within the skin measured over time, obtained from O2C-measurement, when topically applying a heat cream (M1), a comparative compound (M4) and without treatment (M2 and M3). The table indicates the following values a), b) and c) at T(bc)_{base} before applying the heat crème, and at points in time T(bc)₁ to T(bc)₁₃ after the heat crème was applied: a) values of the Vel in arbitrary units (AU) obtained by O2C-measurement; b) calculated change of the Vel in AU with reference to the value obtained at T(bc)_{base}; and c) elapsed time for T(bc)₁ to T(bc)₁₃ in seconds after finishing the application of the heat crème to M1.

FIG. 4 shows the setup and spatial arrangement for the measurements of the temperature on the skin.

FIG. 5 is a diagram showing the baseline temperature on the skin over time without any treatment at M1 to M4. T(t)_{base} is indicated which was taken at 10.8 min. Furthermore, a thermal view of the subject's arms taken at T(t)_{base} is shown.

FIG. 6 is a diagram showing the temperature on the skin measured over time, wherein a topical heat administration (heat pad) at M4 has been used and no heat was administered to M1, M2 and M3. Furthermore, measurement points in time T(tp)_{pad}, T(tp)₁, T(tp)₂ and T(tp)₃ are indicated. T(tp)ₚᵣₑ is before any application of heat, T(tp)_{pad} is during the application of the heat to M4 using a head pad, and T(tp)₁ to T(tp)₃ are thereafter. M1 to M3 were left untreated.

FIG. 7 shows thermal views of the subject's forearms at measurement points in time T(tp)ₚᵣₑ, T(tp)_{pad} and T(tp)₁ to T(tp)₃ as indicated in FIG. 6.

FIG. 8 is a diagram showing the temperature on the skin measured over time when topically administering a heat cream (M1), a comparative compound (M3) and without treatment (M2 and M4). Furthermore, measurement time points T(tc)ₚᵣₑ, T(tc)_{DAC} and T(tc)₁ to T(tc)₉ are indicated: T(tc)ₚᵣₑ is before the application of any comparative composition or test composition (heat crème), T(tc)_{DAC} after the application of the comparative composition to M3, and T(tc)₁ to T(tc)₉ are after the application of the heat crème to M1. M2 and M4 were left untreated.

FIG. 9 shows thermal views of the subject's forearms at measurement points in time T(tc)ₚᵣₑ, T(tc)_{DAC} and T(tc)₁ to T(tc)₉ as indicated in FIG. 8.

FIG. 10 shows a graph of the initial amount of CRP and the development of the amount of CRP over a treatment course of 53 weeks of one patient suffering from rheumatoid arthritis (patient 1), including controls, placebo, incorrect treatment and correct.

FIG. 11 shows the development of the relative amounts of CRP of patients suffering from rheumatoid arthritis (patients 1, 2 and 4 to 6) and polyarthritis (patient 9) over up to 10 weeks of treatment. The initial CRP amount is set to 1 (100%) in each case.

FIG. 12 shows a bar chart of the development of the amount of CRP of three patients suffering from rheumatoid arthritis (patients 1, 2 and 4) after treatment for up to 5 weeks.

FIG. 13 shows a bar chart of the development of the amount of CRP after treatment for up to 53 weeks. All rheumatoid arthritis patients for which CRP values were collected are shown.

FIG. 14 shows photographs of the right and left hand of a patient suffering from rheumatoid arthritis (patient 2). The initial condition is shown and the improved condition after treatment for 1, 2 and 45 weeks.

FIG. 15 shows a bar chart of the development of the HAQ-score of a patient (patient 7) suffering from rheumatoid arthritis and synovitis over a treatment course of 12 weeks.

FIG. 16 shows a bar chart of the development of the amount of CRP and of the HAQ of a patient (patient 9) suffering from polyarthritis over a treatment course of 19 weeks.

FIG. 17 shows a graph of the development of the BASDAI (Bath Ankylosing Spondylitis Disease Activity Index) of two patients (patient 11 and 12) suffering from ankylosing spondylitis (Bechterew's disease) after treatment for 10 weeks.

FIG. 18 shows a bar chart of the development of the SHILD-score of a patient (patient 15) suffering from multiple sclerosis over a treatment period of for 4 years.

FIG. 19 shows photographs taken from the eyes of a patient (patient 1) suffering from Basedow's disease before onset of disease, 2 months after onset of disease and after a treatment for 10 months and 13 months, respectively.

FIG. 20 shows a schematic block-diagram of a medical device.

FIG. 21 shows a schematic block-diagram of another medical device.

FIG. 22 shows a schematic block-diagram of a further medical device.

FIG. 23 shows a schematic block-diagram of an even further medical device.

FIG. 24 shows a schematic block-diagram of a still further medical device.

FIG. 25 shows a schematic sketch of a medical device.

FIG. 26 shows a schematic sketch of another medical device.

### DETAILED DESCRIPTION

The following definitions and preferred embodiments relate generally to any of the embodiments as described herein, even if not explicitly mentioned and even if stated under different topics or outline notes, unless described otherwise.

The present invention is based on the surprising finding that the creation of a vasodilation (blood vessel dilation, particularly a capillary dilation), increasing the blood volume, increasing the sO₂ (oxygen saturation of haemoglobin), increasing the rHb (relative haemoglobin amount), increasing the temperature, generating a redness, administering conditioning energy, administering a skin-conditioning agent within/on/to e.g. the skin needs to be combined with the administration of an immunomodulatory substance(s). Thereby, without wishing to be bound to theory, it is believed that the PBMCs accumulate e.g. within the skin and the PBMCs are brought in close vicinity and/or contact with immunomodulatory substance(s) administered. Further without wishing to be bound to theory, it is believed that the LHs residing within the skin are brought in contact with immunomodulatory substance(s) administered. Further without wishing to be bound to theory, it is believed that peripheral Tregs like skin-resident Foxp3, CD25, CD4 regulatory T cells which reside within the skin are brought in contact with immunomodulatory substance(s) administered. The skin can these ways be used as an in-vivo incubator for the PBMC, LHs and/or peripheral Tregs, thereby it is believed to provide affected PBMCs, LHs and/or peripheral Tregs for effecting a modulated immune system activity.

The general concept of the present invention relates to a method comprising the steps of:
(A-0) generating an accumulation of PBMCs (peripheral blood mononuclear cells) within a body part, preferably the skin of a subject;
(A-1) generating a vasodilation of the capillaries within a body part, preferably the skin of a subject;
(A-2) generating an increased blood volume within a body part, preferably the skin of a subject;
(A-3) generating an increased sO₂ (oxygen saturation of haemoglobin) a body part, preferably the skin, and/or an increased rHb (relative haemoglobin amount) within a body part, preferably the skin of a subject;
(A-4) generating an increased temperature on a body part, preferably the skin of a subject;
(A-5) generating a redness on a body part, preferably the skin of a subject;
(A-6) administering conditioning energy to a body part, preferably the skin of a subject;
(A-7) administering a skin-conditioning agent to a body part, preferably the skin of a subject; and/or
(A-8) administering PBMCs into a body part, preferably the skin of a subject, and further comprising any or both of steps:
(B) administering the immunomodulatory substance(s) to this body part of the subject; and/or
(C) administering the immunomodulatory substance(s) to this body part of the subject, wherein the immunomodulatory substance(s) administered in step (C) is different from the immunomodulatory substance(s) administered in step (B).

Hence, the present invention relates to an immunomodulatory substance(s) and/or a skin-conditioning agent for use in a method for treating and/or preventing an inflammatory disease, immunological disease and/or autoimmunological disease in a subject,
wherein the method comprises a step (A), wherein step (A) is selected from one or more of steps:
   (A-0) generating an accumulation of PBMCs within the skin of the subject;
   (A-1) generating a vasodilation of the capillaries within the skin of the subject;
   (A-2) generating an increased blood volume within the skin of the subject;
   (A-3) generating an increased sO₂ within the skin and/or an increased rHb within the skin of the subject;
   (A-4) generating an increased temperature on the skin of the subject;
   (A-5) generating a redness on the skin of the subject;
   (A-6) administering conditioning energy to the skin of the subject;
   (A-7) administering a skin-conditioning agent to the skin of the subject; and/or
   (A-8) administering PBMCs into the skin of the subject,
and the method further comprises the step(s) of:
   (B) administering the immunomodulatory substance(s) to the skin of the subject; and/or
   (C) administering the immunomodulatory substance(s) to the skin of the subject,
wherein the immunomodulatory substance(s) administered in step (C) is different from the immunomodulatory substance(s) administered in step (B),
and to said method as such.

The present invention also relates to pharmaceutical compositions, injectable dosage forms, topical dosage forms, medical devices and kits of parts according to the independent claims and as mentioned in further detail below.

The present invention is very effective in the treatment and/or prevention of an inflammatory disease, immunological disease and/or autoimmunological disease. The present invention aims, amongst others, at the control of joint inflammation and hence, the prevention or delay of future joint degeneration caused thereby. Advanced joint degeneration often necessitates joint replacement in the long term, which may be delayed or possibly never be required at all.

Furthermore, present invention has no adverse side effects. Thus, the present invention may also contribute to mental and/or physical wellbeing of the treated subjects, may be life-lengthening while at the same time maintaining an improved quality of life.

Furthermore, the present invention is easy and quick to perform, for example, because steps (A) and (B) and/or (C) are performed on and/or within the skin. The skin has the advantage to be easily accessible for treatment and administration. Furthermore advantageous is that treatments on the skin, like balms, crèmes or plasters, or into the skin, like injections, are generally of lower health risk for a subject like for instance intravenous injections.

The present invention provides a ready to use immunotherapeutic on a prescription basis without the requirement of specialized health care centers. This keeps the costs low by at the same time ensuring a high patient compliance. The subjects or owner of an animal may in several embodiments of the present invention self-apply the method steps, which allows for an on-site application of the therapy for immobile humans or non-transportable or transport-unwilling animals. Therefore, a therapy is provided by the present invention which is also accessible to patients which live remotely out of the reach of health care centers.

Further, the present invention may get along without administering subject's own body extracts like body fluids, blood, cells, tissue, PBMCs, substance(s) etc. (except for the case of administering PBMCs). Such extracts may be swapped or contaminated, e.g. during storage, freezing or body-external incubation for instance during cell-culturing. Conventional immunotherapies frequently use cell-culturing, wherein the cell-culturing is performed body-external. The present invention moves the cell-culturing into the subject's body where the incubation takes place thereby using the subject's body, particularly the skin, as an in-vivo incubator. Hence, with the present invention there is no risk of contamination or swapping such extracts or the risk is at least minimized.

Further advantageous, the PBMCs, LHs and/or peripheral Tregs remain in their natural habitat under optimal conditions and in an optimal micro-milieu as it may be for instance provided within the skin. Thereby, the PBMCs, LHs and/or peripheral Tregs are not exposed to any stress due to extraction, freezing, fluctuations in temperature, CO₂-environmental content, nutrient supply and media composition, all of which can result in altered expression patterns or even death of a part of the cell population. Furthermore, expensive staff and sophisticated equipment necessary for convention cell-culturing are dispensable.

Still further advantages are that the therapy can be offered in forms which do not require training of the user or which compensate inadequate training of the user in performing the method. The handling and execution of the method can be designed very simple and straightforward and a correct execution of the method can be ensured. This may be particularly relevant for very young or elderly patients or for users having impaired manual skills as it is for instance frequently the case with rheumatological diseases. Furthermore, fear of self-application errors can be taken away and a lack of treatment adherence can be mitigated or even overcome due to the simple application and uncomplicated availability of the method without a frequent visit to a doctor. The percentage of patients who respond to therapy of the present invention, is very high. The present invention does not provide any adverse side effects or intolerances as known to date. To the contrary, patients even feel stronger and more energetic. Such adverse side effects or intolerances are also not expected even in long-term use. Moreover, a loss of efficacy in long-term use, as is often the case with e.g. biologics, is also not to be expected with the therapeutic approach of the present invention. Hence, the method is highly suitable to find a broad and sustainable public application.

Moreover, the therapy according to the present invention is compatible with conventional preparations, pharmaceuticals and drugs, including biologics, biosimilars, glucocorticoids, Disease modifying antirheumatic drugs (DMARDs) like methotrexate (MTX), hydroxychloroquin (HCQ) and heflunomide (LEF) and confers an effect on top and in addition to these preparations. It may even replace or displace these preparations completely, and that, as stated before, without any side effects, intolerances or long-term damages.

Without wishing to be bound to theory, it is believed that the effect of the present invention is mainly conferred by regulatory T-cells and/or helper T-cells or a subset thereof which may be presumably generated when applying the method of the present invention. The present invention is believed to be based, without whishing to be bound thereto, on the following principles: the skin tissue provides a tight and firm structure. Thereby, it is believed that skin tissue may trap, immobilize, anchor, entangle and/or hold PBMCs, that are a believed to be accumulated, without being flushed away. Furthermore, it is believed that the skin tissue may trap, immobilize, anchor, entangle and/or hold the administered immunomodulatory substance(s) and the immunomodulatory substance(s) may not be flushed away. Thereby a sufficiently long contact of the PBMCs, LHs and/or peripheral Tregs with the immunomodulatory substance(s) is provided while using the subject's body as an in-vivo incubator leading directly or indirectly to the development of regulatory T-cells and/or helper T-cells or a subset thereof. The principles, again without whishing to be bound thereto, are be belived to be in detail as follows:
The blood supply of the sub-topical skin layers and particularly the dermis essentially provided by capillary vessels. In contrast to veins and arteries, capillaries have in their normal state a very narrow cross section. Solely erythrocytes and thrombocytes, which are deprived of a nucleus, are compressible and flexible enough to enter and stream through non-dilated capillaries thereby ensuring the skin's oxygen supply and skin integrity.
PBMCs are nucleated cells, that is, they contain a nucleus. Therefore, it is believed that PBMCs are too bulky to squeeze through non-dilated capillaries. As a consequence, it is believed that PBMCs, particularly naïve lymphocytes like naïve T-cells, are not present or at least not present in effective amounts within the skin capillaries and hence, within the skin. In other words, it is believed that the skin tissue is normally free or essentially free of blood-derived PBMCs and does not contain blood-derived PBMCs in an effective amount. Hence, it is believed that the skin, that is skin as a whole including capillaries and tissue, normally contains only a very limited amount of PBMCs compared to e.g. the vascular system or the lymph nodes.
Therefore, in order to generate an accumulation of PBMCs within the skin, particularly within the skin tissue, beside others, PBMCs might be, for instance, injected directly into the skin tissue as for instance in step (A-8).
Another possibility, again without whishing to be bound to that theory, which is believed to generate an accumulation of PBMCs within the skin comprises two things. First, the PBMCs must be given access to the capillaries to bring them in the vicinity of the adjacent skin tissue (effected by e.g. any of steps (A-1) to (A-7)). Thereby an accumulation of PBMCs within the skin of the subject is believed to be generated, particularly within the lumen of the capillaries of the skin. Secondly, the PBMCs require to be caused to leave the capillaries, cross the capillary wall and migrate into the adjacent skin tissue. This migration mechanism is known to naturally occur and does not require further action or affectation. In brief, for crossing the vascular endothelial of the capillaries, PBMCs, e.g. lymphocytes, start tethering and rolling to a complete stop inside along the capillary walls and subsequently cross through the capillary wall thereby migrating from the capillary lumen into the adjacent skin tissue. Hence, according to the theory, but again without whishing to be bound thereto, apart from administering PBMCs e.g. by injection, also e.g. any of below steps (A-1) to (A-7) may generate an accumulation of PBMCs within the skin, particularly within the lumen of the skin capillaries and finally the skin tissue due to the natural migration of the PBMCs out of the capillaries into the skin tissue.
After accumulation, i.e. migration and/or administration into the skin tissue, the PBMCs are, unlike in blood, believed to be trapped and hold by the skin tissue and cannot be flushed away.
Similarly, the immunomodulatory substance(s) administered to the skin (e.g. steps (B) and/or (C)) are not flushed away and diluted by the blood and stay in place, at least for a certain time limited by their diffusion rates.
The trapping and holding of at the same time the immunomodulatory substance(s) and PBMCs allows to incubate the PBMCs, e.g. lymphocytes, more particularly naïve lymphocytes like naïve T-cells, for a time sufficient with the immunomodulatory substance(s). Thereby, the PBMCs are incubated in vivo for (further) affecting the PBMCs, like for instance regulating, inducing, pre-activating, activating, suppressing, maturing, differentiating, modulating and/or proliferating PBMCs. The administered immunomodulatory substance(s) may be capable to effect the desired affectation of the PBMCs.
Moreover, within the skin tissue dendritic cells like LHs inherently reside, particularly within the basal part of the epidermis, but they are not present within the blood. It is believed that such dendritic cells, further again without wishing to be bound to theory, are also capable of affecting the PBMCs. Such affectation by dendritic cells may be in addition to, in combination with, in an enhancing manner and/or synergistically with the immunomodulatory substance(s). Hence, beside other, LHs may provide an optimal or advantageous micro-milieu supporting PBMCs affection, however LHs cannot replace or compensate for the administration of immunomodulatory substance(s).
After affecting the PBMCs (e.g. the naïve PBMCs), affected PBMCs, particularly regulatory T-cells, which are no longer naïve, naturally leave the skin, enter the blood stream and migrate to their place of action in the subject's body without any further action or affectation. The place of action may be for instance an inflamed joint in the subject's body. It is believed that the effect of the present invention is mainly conferred by affected PBMCs, particularly regulatory T-cells and/or helper T-cells or a subset thereof.
It may be noted that in addition to affecting the PBMCs, the immunomodulatory substance(s) like e.g. INF-y may, but not necessarily, functions as an attractor creating a micro-milieu which facilitates the naturally occurring process of migration of the PBMCs from the capillary lumen across the capillary wall into the surrounding tissue. Nevertheless, this does not replace the fact that prior to crossing the capillary walls the PBMCs need to be accumulated within the skin capillaries or skin capillary lumen. This requires other means than administering the immunomodulatory substance(s) and can be effected by e.g. any of steps (A-1) to (A-7). As already mentioned above, the skin capillaries are in their non-dilated ground state too narrow for PBMCs to squeeze into and through them. The immunomodulatory substance(s) administered in the present invention do not provide for the presence of PBMCs within the capillaries of the skin.
As already mentioned above, LHs and peripheral Tregs are cells inherently residing within the skin, particularly within the basal part of the epidermis. Without wishing to be bound to theory, it is believed that in the blood of patients suffering from an inflammatory disease, immunological disease and/or autoimmunological disease like for instance rheumatoid arthritis, low amounts of autoantigens like RA-autoantigen/autoantibody immune complexes are present. It is believed that in case LHs get in contact with such low amounts of autoantigens immune suppression, self-tolerance and the reduction of the immune response is initiated and finally activation of CD4 regulatory T cells (T_{reg}s) can occur in the lymph nodes.
Without wishing to be bound to theory, it is believed that these autoantigens are delivered and accumulated within the skin by for instance creating a vasodilation, particularly a capillary dilation within the skin, increasing the blood volume, sO₂ or rHb within the skin, increasing the temperature on the skin, generating a redness on the skin or by administering conditioning energy or administering a skin-conditioning agent to the skin. It is believed that these autoantigens are efficiently cross-presented to the peripheral Tregs (like Foxp3, CD25, CD4 T cells) which may, in antigen specific manner, support development of the immuno-suppressive phenotype of the LHs finally leading to activation of regulatory CD4 suppressor T cells (T_{reg}s) within the lymph nodes. The intrinsic capability of LHs to sense self versus foreign antigens drives in the case of acquisition of autoantigen their differentiation toward the development of immuno-suppressive phenotype. The immunolatory substance(s), like e.g. INF-y or IL-2, particularly in ultra-low dosages, may affect the LHs and/or peripheral T_{reg}s thereby promoting activation and maturation of the immunosuppressive phenotype of the LHs.
Again without wishing to be bound to theory, it is in particular believed that via their Fc-receptors LHs internalize the autoantigen/autoantibody-complexes present in low amounts in the patient's blood and process the autoantigen for surface presentation (MHCII-antigen complex). In case of generating an increased temperature on the skin, it is believed that such warming may have a further effect of causing the LHs to generate heat shock proteins (chaperones) which may stabilize the MHC-autoantigen complex, thereby promoting effective antigen presentation. The resulting LHs which carry the autoantigen as MHCII-autoantigen-complex are believed to move to the lymph nodes and present the autoantigen to CD4 T helper cells. CD4 T helper cells recognize the autoantigen as self and finally activate the already pre-activated LHs carrying autoantigen towards immunosuppression and tolerance for this autoantigen. The final distinction between self versus foreign antigens is believed to be made here. Without wishing to be bound thereto, it is believed that based in that theory immunosuppressive and tolerogenically activated LHs are generated within the lymph nodes. These immunosuppressive and tolerogenically activated LHs presumably then activate regulatory CD4 suppressor T cells (T_{reg}s) by autoantigen cross presentation within the lymph nodes.

As already mentioned above, it is believed that also the PBMCs that are believed to be accumulated within skin and brought in contact with the immunomodulatory substance(s) finally lead to the generation of T_{reg}s. Both theories, that is, via the PBMCs or via the LHs and the peripheral T_{reg}s, have in common that finally T_{reg}s may be generated. These T_{reg}s are then believed to spread, whereby the tolerogenic and immunosuppressive effect is expanded and globalized within the subject's body. The production of pro-inflammatory cytokines is lowered, pro-inflammatory cytotoxic T-cells is counteracted in an immunosuppressive manner, self-tolerance is developed and inflammatory responses, immune responses or autoimmune responses are downregulated. The inflammatory disease, immunological disease and/or autoimmunological disease is controlled.

As a consequence, the treatment is local while the effect is systemic.

For the above reasons, the skin is highly suitable to be used as an in-vivo incubator for PBMCs, LHs and/or peripheral T_{reg}s in order to generate helper T-cells, or a subset thereof, and/or regulatory T-cells.

The present invention relates to an immunomodulatory substance(s) and/or a skin-conditioning agent for use in a method for treating and/or preventing an inflammatory disease, immunological disease and/or autoimmunological disease in a subject,
wherein the method comprises, preferably consists of, a step (A), wherein step (A) is selected from one or more of steps:
(A-0) generating an accumulation of PBMCs within the skin of the subject;
(A-1) generating a vasodilation of the capillaries within the skin of the subject;
(A-2) generating an increased blood volume within the skin of the subject;
(A-3) generating an increased sO₂ within the skin and/or an increased rHb within the skin of the subject;
(A-4) generating an increased temperature on the skin of the subject;
(A-5) generating a redness on the skin of the subject;
(A-6) administering conditioning energy to the skin of the subject;
(A-7) administering a skin-conditioning agent to the skin of the subject; and/or
(A-8) administering PBMCs into the skin of the subject,
and the method further comprises, preferably further consists of, a step (B):
(B) administering the immunomodulatory substance(s) to the skin of said subject,
Or
wherein the method comprises, preferably consists of, a step (A), wherein step (A) is selected from one or more of steps:
(A-0) generating an accumulation of PBMCs within the skin of the subject;
(A-1) generating a vasodilation of the capillaries within the skin of the subject;
(A-2) generating an increased blood volume within the skin of the subject;
(A-3) generating an increased sO₂ within the skin and/or an increased rHb within the skin of the subject;
(A-4) generating an increased temperature on the skin of the subject;
(A-5) generating a redness on the skin of the subject;
(A-6) administering conditioning energy to the skin of the subject;
(A-7) administering a skin-conditioning agent to the skin of the subject; and/or
(A-8) administering PBMCs into the skin of the subject,
and the method further comprises, preferably further consists of, a step (C);
(C) administering the immunomodulatory substance(s) to the skin of said subject,
Or
wherein the method comprises, preferably consists of, a step (A), wherein step (A) is selected from one or more of steps:
(A-0) generating an accumulation of PBMCs within the skin of the subject;
(A-1) generating a vasodilation of the capillaries within the skin of the subject;
(A-2) generating an increased blood volume within the skin of the subject;
(A-3) generating an increased sO₂ within the skin and/or an increased rHb within the skin of the subject;
(A-4) generating an increased temperature on the skin of the subject;
(A-5) generating a redness on the skin of the subject;
(A-6) administering conditioning energy to the skin of the subject;
(A-7) administering a skin-conditioning agent to the skin of the subject; and/or
(A-8) administering PBMCs into the skin of the subject,
and the method further comprises, preferably further consists of, the steps;
(B) administering the immunomodulatory substance(s) to the skin of said subject; and
(C) administering the immunomodulatory substance(s) to the skin of the subject,
wherein the immunomodulatory substance(s) administered in step (C) is different from the immunomodulatory substance(s) administered in step (B), and to any of said methods as such.

In a preferred aspect of all embodiments of the present invention described herein, the present invention relates to immunomodulatory substance(s) and/or a skin-conditioning agent for use in a method for treating and/or preventing an inflammatory disease, immunological disease and/or autoimmunological disease in a subject,
wherein the method comprises, preferably consists of, a step (A), wherein step (A) is selected from one or more of steps:
(A-0) generating an accumulation of PBMCs within the skin of the subject;
(A-1) generating a vasodilation of the capillaries within the skin of the subject;
(A-2) generating an increased blood volume within the skin of the subject;
(A-3) generating an increased sO₂ within the skin and/or an increased rHb within the skin of the subject;
(A-4) generating an increased temperature on the skin of the subject;
(A-5) generating a redness on the skin of the subject;
(A-6) administering conditioning energy to the skin of the subject;
(A-7) administering a skin-conditioning agent to the skin of the subject; and/or
(A-8) administering PBMCs into the skin of the subject,
and the method further comprises, preferably further consists of, a step (B):
(B) administering the immunomodulatory substance(s) to the skin of said subject, or wherein the method comprises, preferably consists of, a step (A), wherein step (A) is selected from one or more of steps:
(A-0) generating an accumulation of PBMCs within the skin of the subject;
(A-1) generating a vasodilation of the capillaries within the skin of the subject;
(A-2) generating an increased blood volume within the skin of the subject;
(A-3) generating an increased sO₂ within the skin and/or an increased rHb within the skin of the subject;
(A-4) generating an increased temperature on the skin of the subject;
(A-5) generating a redness on the skin of the subject;
(A-6) administering conditioning energy to the skin of the subject;
(A-7) administering a skin-conditioning agent to the skin of the subject; and/or
(A-8) administering PBMCs into the skin of the subject,
and the method further comprises, preferably further consists of, the steps;
(B) administering the immunomodulatory substance(s) to the skin of said subject; and
(C) administering the immunomodulatory substance(s) to the skin of the subject,
wherein the immunomodulatory substance(s) administered in step (C) is different from the immunomodulatory substance(s) administered in step (B),
and to any of said methods as such.

In a more preferred aspect of all embodiments of the present invention described herein, the present invention relates to immunomodulatory substances and/or a skin-conditioning agent for use in a method for treating and/or preventing an inflammatory disease, immunological disease and/or autoimmunological disease in a subject,
wherein the method comprises, preferably consists of, a step (A), wherein step (A) is selected from one or more of steps:
(A-0) generating an accumulation of PBMCs within the skin of the subject;
(A-1) generating a vasodilation of the capillaries within the skin of the subject;
(A-2) generating an increased blood volume within the skin of the subject;
(A-3) generating an increased sO₂ within the skin and/or an increased rHb within the skin of the subject;
(A-4) generating an increased temperature on the skin of the subject;
(A-5) generating a redness on the skin of the subject;
(A-6) administering conditioning energy to the skin of the subject;
(A-7) administering a skin-conditioning agent to the skin of the subject; and/or
(A-8) administering PBMCs into the skin of the subject, and the method further comprises, preferably further consists of, the steps;
(B) administering the immunomodulatory substance(s) to the skin of said subject; and
(C) administering the immunomodulatory substance(s) to the skin of the subject,
wherein the immunomodulatory substance(s) administered in step (C) is different from the immunomodulatory substance(s) administered in step (B),
and to any of said methods as such.

For the sake of simplicity and clarity, several of the following embodiments refer to step (A) in general. However, if not mentioned otherwise, it is to be understood that any of these embodiments refers independently to any of steps (A-0), (A-1), (A-2), (A-3), (A-4), (A-5), (A-6), (A-7) and/or (A-8).

The immunomodulatory substance(s) administered in step (B) is or may also be denoted as first immunomodulatory substance(s), the immunomodulatory substance(s) administered in step (C) is or may also be denoted as second immunomodulatory substance(s) and the immunomodulatory substance(s) administered in step (B₁) (see below) is or may also be denoted as third immunomodulatory substance(s). If not mentioned otherwise, it is to be understood that any embodiment or definition described herein in terms of the immunomodulatory substance(s) is independently and mutatis mutandis applicable to the first immunomodulatory substance(s), the second immunomodulatory substance(s) and the third immunomodulatory substance(s) in any of the embodiments described herein, particular to any of the embodiments of the medical device described herein.

It is to be understood that the expressions “first”, “second” and “third” as mentioned in any of the embodiments described herein in respect of the immunomodulatory substance(s) do not intent or suggest any order, hierarchy or prioritization of any of the first immunomodulatory substance(s), the second immunomodulatory substance(s) and the third immunomodulatory substance(s) (also denoted as immunomodulatory substance(s)). These expressions only serve to unambiguously assign, designate and refer to the corresponding immunomodulatory substance(s). Similarly, it is to be understood that in any of the embodiments described herein the immunomodulatory substance(s) of step (B), step (C) and step (B₁) may also be regarded as “first immunomodulatory substance(s)” (step (B)), “second immunomodulatory substance(s)” step (C) and “third immunomodulatory substance(s)” step (B₁) as mentioned in any of the embodiments described herein.

It is to be understood that the “immunomodulatory substance(s) for use in a method for treating and/or preventing an inflammatory disease, immunological disease and/or autoimmunological disease in a subject” as mentioned in any of the embodiments described herein is the immunolatory substance(s) of step (B), step (B₁) and step (C), respectively, depending on what is applicable.

It is further to be understood in any of the embodiments described herein that in case the immunomodulatory substance(s) of step (B) is a “first immunomodulatory substance(s)”, the immunomodulatory substance(s) of step (C) is a “second immunomodulatory substance(s)” and/or the immunomodulatory substance(s) of step (B₁) is a “third immunomodulatory substance(s)”, the “immunomodulatory substance (s) for use in a method for treating and/or preventing an inflammatory disease, immunological disease and/or autoimmunological disease in a subject” as mentioned in any of the embodiments described herein is a first immunomodulatory substance(s), a second immunomodulatory substance(s) and a third immunomodulatory substance(s), respectively, for use in a method for treating and/or preventing an inflammatory disease, immunological disease and/or autoimmunological disease in a subject, depending on what is applicable.

Preferably, the inflammatory disease, immunological disease and/or autoimmunological disease to be treated and/or prevented in any of the embodiments described herein requires for treating and/or preventing a modulation of the subject's immune system, wherein the modulation of the immune system may be a downregulation of the immune system.

The downregulation may be for instance an anti-inflammatory regulation and/or a less aggressive regulation in recognizing antigen, autoantigen or antigen presenting cells, thereby providing e.g. a reduction in inflammatory activity and swelling.

It is preferred that the inflammatory disease, immunological disease and/or autoimmunological disease to be treated and/or prevented as mentioned in any of the embodiments described herein:
requires for treating and/or preventing a downregulation of the immune system, more preferably of the subject's immune system; and/or
is characterized by a symptomatic caused by a too aggressive and/or hyperactive immunoreaction of the subject's body; and/or involves an overrepresentation and/or hyperactivity of cytotoxic T-cells; and/or
is not caused by a genetic condition of the subject; and/or
has an immunological background and/or an autoimmunological background. More preferably, the inflammatory disease, immunological disease and/or autoimmunological disease to be treated and/or prevented is an immunological disease, autoimmunological disease and/or an organ rejection reaction after organ transplantation, even more preferably an immunological disease and/or autoimmunological disease. It may be pointed out that an inflammatory disease may have an immunological background or an autoimmunological background, while an immunological disease or autoimmunological disease must not necessarily be associated with an inflammation; and/or
is associated with an inflammation.

More preferably, the inflammatory disease, immunological disease and/or autoimmunological disease to be treated and/or prevented as mentioned in any of the embodiments described herein comprises, preferably consists of:
arthritis,
preferably as defined in the preferred embodiments of the arthritis described herein;
synovitis and/or tenosynovitis,
preferably in accordance with code M65.- of the standard ICD-10-GM 2021;
inflammatory rheumatic diseases,
preferably as defined in the preferred embodiments of the inflammatory rheumatic diseases described herein;
Basedow's disease (also called Graves' disease), preferably in accordance with code E05.0 of the standard ICD-10-GM 2021;
Hashimoto's disease (also called Hashimoto's thyroiditis), preferably in accordance with code E06.3 of the standard ICD-10-GM 2021;
inflammatory diseases of the nervous system, including, but not limited to:
   multiple sclerosis,
   preferably in accordance with code G35.-, more preferably in accordance with code G35.10 of the standard ICD-10-GM 2021;
Morbus Crohn,
preferably in accordance with code K50.- of the standard ICD-10-GM 2021;
Uveitis ante or a and/or iridocyclitis,
preferably in accordance with codes H20.0, H20.1 and H20.9 of the standard ICD-10-GM 2021;
diabetic diseases like Diabetes mellitus type 1, preferably in accordance with code E10.- of the standard ICD-10-GM 2021;
myasthenia gravis, preferably in accordance with code G70.0 of the standard ICD-10-GM 2021;
glomerulonephritis based on autoimmunological background, e.g. not caused by genetic or bacterial background, preferably in accordance with code N05.- of the standard ICD-10-GM 2021;
hepatitis, including
   autoimmune hepatitis, preferably in accordance with code K75.4 of the standard ICD-10-GM 2021; and/or
   hepatitis C, preferably in accordance with code B18.2 of the standard ICD-10-GM 2021;
APECED (also called autoimmune polyendocrine syndrome type 1),
preferably in accordance with code E31.0 of the standard ICD-10-GM 2021;
Goodpasture's syndrome, preferably in accordance with code M31.- of the standard ICD-10-GM 2021;
Polyneuritides, preferably in accordance with code G61.- of the standard ICD-10-GM 2021, including:
   CIDP (also called chronic inflammatory demyelinating polyneuropathy),
   preferably in accordance with code G61.8 of the standard ICD-10-GM 2021; and/or
   Guillain-Barré syndrome,
   preferably in accordance with code G61.0 of the standard ICD-10-GM 2021;
Lichen mucosae,
preferably in accordance with code L43.- of the standard ICD-10-GM 2021; and/or
Stiff-Person syndrome,
preferably in accordance with code G25.88 of the standard ICD-10-GM 2021.

The expression “standard ICD-10-GM 2021” as mentioned in any of the embodiments described herein refers to the “ICD-10-GM 2021 Systematisches Verzeichnis: Internationale statistische Klassifikation der Krankheiten and verwandter Gesunctheitsprobleme, 10. Revision—German Modification”, Deutscher Arzteverlag, ISBN-13: 978-3-7691-3722-4.

Even more preferably, the inflammatory disease, immunological disease and/or autoimmunological disease to be treated and/or prevented comprises, preferably consists of:
arthritis,
preferably as defined in the preferred embodiments of the arthritis described herein;
synovitis and/or tenosynovitis,
preferably in accordance with code M65.- of the standard ICD-10-GM 2021;
inflammatory rheumatic diseases,
preferably as defined in the preferred embodiments of the inflammatory rheumatic diseases described herein;
Hashimoto's disease (also called Hashimoto's thyroiditis),
preferably in accordance with code E06.3 of the standard ICD-10-GM 2021;
Basedow's disease (also called Graves' disease),
preferably in accordance with code E05.0 of the standard ICD-10-GM 2021;
Morbus Crohn,
preferably in accordance with code K50.- of the standard ICD-10-GM 2021;
and/or
inflammatory diseases of the nervous system, including, but not limited to:
   multiple sclerosis,
   preferably in accordance with code G35.-, more preferably in accordance with code G35.10 of the standard ICD-10-GM 2021.

Preferably, the arthritis as mentioned in any of the embodiments described herein comprises, preferably consists of, monoarthritis, oligoarthritis and/or polyarthritis, more preferably is a polyarthritis, preferably in accordance with codes M13.-, M14.-, M15.- and/or M25.-, more preferably code M25.5 of the standard ICD-10-GM 2021.

Preferably, the synovitis and/or tenosynovitis as mentioned in any of the embodiments described herein comprises, preferably consists of, synovitis and tenosynovitis, more preferably, synovitis, preferably in accordance with code M65.- of the standard ICD-10-GM 2021.

Preferably, the inflammatory rheumatic disease as mentioned in any of the embodiments described herein comprises, preferably consists of:
rheumatoid arthritis (RA) (also called chronic polyarthritis),
preferably in accordance with code M05.-, more preferably code M05.80 of the standard ICD-10-GM 2021;
juvenile arthritis,
preferably in accordance with code M08.- of the standard ICD-10-GM 2021;
spondyloarthritides including:
   Bechterew's disease (also called ankylosing spondylitis),
   preferably in accordance with code M45.-, of the standard ICD-10-GM 2021,
   psoriatic arthritis,
   preferably in accordance with code M07.- of the standard ICD-10-GM 2021;
   enteropathic arthritis (associated with intestinal diseases such as ulcerative colitis);
   preferably in accordance with code M07.- of the standard ICD-10-GM 2021;
   reactive arthritis;
   preferably in accordance with code M02.- of the standard ICD-10-GM 2021; and/or
   undifferentiated spondyloarthritis;
collagenoses (connective tissue diseases) like:
   systemic sclerosis,
   preferably in accordance with code M34.9 of the standard ICD-10-GM 2021;
   Sjögren's syndrome,
   preferably in accordance with code M35.0 of the standard ICD-10-GM 2021;
   polymyositis,
   preferably in accordance with code M33.- of the standard ICD-10-GM 2021;
   dermatomyositis and mixed collagenosis,
   preferably in accordance with code M30-M36 of the standard ICD-10-GM 2021; and/or
   lupus erythematosus,
   preferably in accordance with code L93.- of the standard ICD-10-GM 2021;
vasculitides (diseases with vascular inflammation),
preferably in accordance with code L93.-, L95.- or M05.- of the standard ICD-10-GM 2021;
polymyalgia rheumatica,
preferably in accordance with code M35.3 of the standard ICD-10-GM 2021;
SAPHO syndrome (also called Synovitis, Acne, Pustulosis, Hyperostosis, Osteitis syndrome), preferably in accordance with code M86.3 of the standard ICD-10-GM 2021;
polychondritis,
preferably in accordance with code M94,1 the standard ICD-10-GM 2021;
myositis fibrosa generalisata,
preferably in accordance with code G71.- of the standard ICD-10-GM 2021;
rigid spine syndrome,
preferably in accordance with code G71.2 of the standard ICD-10-GM 2021;
neuromyotonie, including:
   Isaac's syndrome,
   preferably in accordance with code G71.1 of the standard ICD-10-GM 2021 and/or
   Morvan's syndrome),
   preferably in accordance with code G60.8 of the standard ICD-10-GM 2021;
and/or
Cramping disease (Satoyoshi syndrome),
preferably in accordance with code M35.8 of the standard ICD-10-GM 2021.

More preferably, the inflammatory rheumatic disease is rheumatoid arthritis, psoriatic arthritis, polymyalgia rheumatica, vasculitis, and/or Bechterew's disease, even more preferably rheumatoid arthritis, polymyalgia rheumatica and/or Bechterew's disease.

More preferably the inflammatory disease, immunological disease and/or autoimmunological disease to be treated and/or prevented comprises, preferably consists of, arthritis, synovitis, tenosynovitis, inflammatory rheumatic disease, Hashimoto's disease, Basedow's disease, Morbus Crohn and/or multiple sclerosis, even more preferably arthritis, synovitis, tenosynovitis, rheumatoid arthritis, polymyalgia rheumatica, vasculitis, Bechterew's disease, psoriatic arthritis, Hashimoto's disease, Basedow's disease, Morbus Crohn and/or multiple sclerosis, still even more preferably polyarthritis, synovitis, rheumatoid arthritis, polymyalgia rheumatica, Bechterew's disease, Basedow's disease and/or multiple sclerosis.

The inflammatory disease, immunological disease and/or autoimmunological disease to be treated and/or prevented and any of the preferred embodiments thereof may be, preferably, are defined and/or determined by any method(s) and/or parameter(s) commonly used in the art and known to the person skilled in the art. Preferably, the definition and/or determination is in accordance with the standard ICD-10-GM 2021, preferably in accordance with the code of the standard ICD-10-GM 2021 indicated as preferred for the embodiment of the inflammatory disease, immunological disease and/or autoimmunological disease and the preferred embodiments thereof.

More preferably, the definition and/or determination of the arthritis, even more preferably the monoarthritis, oligoarthritis and/or polyarthritis, is in accordance with the standard ICD-10-GM 2021, preferably with codes M13.-, M14.-, M15.- and/or M25.-, more preferably with code M25.5 of the standard ICD-10-GM 2021. Still even more preferably, the definition and/or determination of the polyarthritis in accordance with the standard ICD-10-GM 2021, preferably with code M13.-, M14.-, M15.- and/or M25.-, more preferably with code M25.5 of the standard ICD-10-GM 2021.

More preferably, the definition and/or determination of the synovitis and/or tenosynovitis in accordance with the standard ICD-10-GM 2021, even more preferably with code M65.- of the standard ICD-10-GM 2021.

More preferably, the definition and/or determination of the inflammatory rheumatic disease is in accordance with the standard ICD-10-GM 2021 indicated as preferred for the embodiment of the inflammatory rheumatic disease and the preferred embodiments thereof.

Hence, even more preferably, the definition and/or determination of the rheumatoid arthritis in accordance with the standard ICD-10-GM 2021, preferably with code M05.-, more preferably with code M05.80 in accordance with the standard ICD-10-GM 2021.

Even more preferably, the definition and/or determination of the polymyalgia rheumatica is in accordance with the standard ICD-10-GM 2021, preferably with code M35.3 of the standard ICD-10-GM 2021.

Even more preferably, the definition and/or determination of the Bechterew's disease is in accordance with the standard ICD-10-GM 2021, preferably with code M45.- of the standard ICD-10-GM 2021.

Even more preferably, the definition and/or determination of the psoriatic arthritis in accordance with the standard ICD-10-GM 2021, preferably with code M07.- of the standard ICD-10-GM 2021.

More preferably, the definition and/or determination of the Basedow's disease is in accordance with the standard ICD-10-GM 2021, preferably with code E05.0 of the standard ICD-10-GM 2021.

More preferably, the definition and/or determination of the Hashimoto's thyroiditis in accordance with the standard ICD-10-GM 2021, preferably with code E06.3 of the standard ICD-10-GM 2021.

More preferably, the definition and/or determination of the multiple sclerosis in accordance with the standard ICD-10-GM 2021, preferably with code G35.-, more preferably with code G35.10 of the standard ICD-10-GM 2021.

The subject as mentioned in any of the embodiments described herein may be, preferably, is any subject in need of the treatment and/or prevention.

The subject may be, preferably, is as mentioned in any of the embodiments described herein any subject having an immune system capable of rising an adaptive immune response, preferably, capable of rising a T-cell immune response.

Preferably, the subject is a vertebrate, more preferably a mammal, even more preferably any of the genus and/or species human or a mammal animal which is selected from cow, buffalo, horse, donkey, elephant, sheep, goat, pig, rabbit, mouse, rat, camel, dromedary, lama, alpaca, dog and/or cat. Still more preferably the subject is a human.

The subject may be, preferably, is as mentioned in any of the embodiments described herein a newborn, suckling, infant child, adolescent and/or adult of the subject, preferably a suckling, infant, child, adolescent and/or adult, still more preferably an infant, child, adolescent or adult, still more preferably a child, adolescent and/or adult, still even more preferably an adolescent and/or adult and further preferably an adult. In case the subject is a human, a newborn is until 28^{th} day of life, a suckling from the beginning of the 29^{th} day of life until the end of the 12^{th} month of life, an infant from the beginning of the 13^{th} month to the completed 3^{rd} year of life, a child from the beginning of the 4^{th} year to the completed 12^{th} year of life, an adolescent from the beginning of the 13^{th} year to the completed 18^{th} year of life and an adult from the beginning of the 19^{th} year of life.

Preferably, the subject is a subject being diagnosed with the inflammatory disease, immunological disease and/or autoimmunological disease and/or a subject for which it is indicative to be at risk to develop the inflammatory disease, immunological disease and/or autoimmunological disease.

Preferably, the subject being diagnosed with the inflammatory disease, immunological disease and/or autoimmunological disease is a subject diagnosed with arthritis, synovitis, tenosynovitis, inflammatory rheumatic disease, Hashimoto's disease, Basedow's disease and/or multiple sclerosis, even more preferably arthritis, synovitis, tenosynovitis, rheumatoid arthritis, polymyalgia rheumatica, Bechterew's disease, psoriatic arthritis, Hashimoto's disease, Basedow's disease and/or multiple sclerosis, still even more preferably polyarthritis, synovitis, rheumatoid arthritis, polymyalgia rheumatica, Bechterew's disease, Basedow's disease and/or multiple sclerosis.

Preferably, the subject for which it is indicative to be at risk to develop the inflammatory disease, immunological disease and/or autoimmunological disease, is a subject for which it is indicative to be at risk to develop arthritis, synovitis, tenosynovitis, inflammatory rheumatic disease, Hashimoto's disease, Basedow's disease and/or multiple sclerosis, even more preferably arthritis, synovitis, tenosynovitis, rheumatoid arthritis, polymyalgia rheumatica, Bechterew's disease, psoriatic arthritis, Hashimoto's disease, Basedow's disease and/or multiple sclerosis, still even more preferably polyarthritis, synovitis, rheumatoid arthritis, polymyalgia rheumatica, Bechterew's disease, Basedow's disease and/or multiple sclerosis.

Usually, in a subject being diagnosed with the inflammatory disease, immunological disease and/or autoimmunological disease, the symptoms of the inflammatory disease, immunological disease and/or autoimmunological disease are already apparent and the subject suffers from the symptoms of the inflammatory disease, immunological disease and/or autoimmunological disease. In other words, the inflammatory disease, immunological disease and/or autoimmunological disease has already been broken out in the subject.

Usually, in a subject for which it is indicative to be at risk to develop the inflammatory disease, immunological disease and/or autoimmunological disease, the symptoms of the inflammatory disease, immunological disease and/or autoimmunological disease are not yet apparent and the subject does not suffer from the symptoms of the inflammatory disease, immunological disease and/or autoimmunological disease. In other words, the inflammatory disease, immunological disease and/or autoimmunological disease has not yet been broken out in the subject. To be at risk is to be understood in the sense that the subject will develop and/or will develop with a high chance the inflammatory disease, immunological disease and/or autoimmunological disease. This may for instance be the case for subjects carrying a HLA-B*27 allel, which may be indicative to be at risk to develop, for instance, Morbus Bechterew, rheumatoid arthritis, Morbus Crohn, reactive arthritis or juvenile arthritis or in case in the subject's family history for instance rheumatoid arthritis has frequently occured.

The diagnosis of the subject being diagnosed with the inflammatory disease, immunological disease and/or autoimmunological disease as mentioned in any of the embodiments described herein may be, preferably, is established by any method(s) and/or parameter(s) commonly used in the art and known to the person skilled in the art. Preferably, the diagnosis established in accordance with the standard ICD-10-GM 2021, preferably in accordance with the code of the standard ICD-10-GM 2021 indicated as preferred for the embodiment of the inflammatory disease, immunological disease and/or autoimmunological disease and the preferred embodiments thereof.

More preferably, the diagnosis of the subject being diagnosed with arthritis, even more preferably the monoarthritis, oligoarthritis and/or polyarthritis, is established in accordance with the standard ICD-10-GM 2021, preferably with codes M13.-, M14.-, M15.- and/or M25.-, more preferably with code M25.5 of the standard ICD-10-GM 2021. Even more preferably, the diagnosis of the subject being diagnosed with polyarthritis established in accordance with the standard ICD-10-GM 2021, preferably M13.-, M14.-, M15.- and/or M25.-, more preferably M25.5 in accordance with the standard ICD-10-GM 2021.

More preferably, the diagnosis of the subject being diagnosed with the synovitis and tenosynovitis established in accordance with the standard ICD-10-GM 2021, even more preferably with code M65.- of the standard ICD-10-GM 2021.

More preferably, the diagnosis of the subject being diagnosed with the inflammatory rheumatic disease is established in accordance with the standard ICD-10-GM 2021 indicated as preferred for the embodiment of the inflammatory rheumatic disease and the preferred embodiments thereof.

Hence, even more preferably, the diagnosis of the subject being diagnosed with rheumatoid arthritis established in accordance with the standard ICD-10-GM 2021, preferably M05.-, more preferably M05.80 in accordance with the standard ICD-10-GM 2021.

Even more preferably, the diagnosis of the subject being diagnosed with polymyalgia rheumatica is established in accordance with the standard ICD-10-GM 2021, preferably M35.3, in accordance with the standard ICD-10-GM 2021.

Even more preferably, the diagnosis of the subject being diagnosed with Bechterew's disease is established in accordance with the standard ICD-10-GM 2021, preferably M45.-, in accordance with the standard ICD-10-GM 2021.

Even more preferably, the diagnosis of the subject being diagnosed with psoriatic arthritis established in accordance with the standard ICD-10-GM 2021, preferably with code M07.- of the standard ICD-10-GM 2021.

More preferably, the diagnosis of the subject being diagnosed with Basedow's disease is established in accordance with the standard ICD-10-GM 2021, preferably M45.-, in accordance with the standard ICD-10-GM 2021.

More preferably, the diagnosis of the subject being diagnosed with Hashimoto's thyroiditis established in accordance with the standard ICD-10-GM 2021, preferably M45.-, in accordance with the standard ICD-10-GM 2021.

More preferably, the diagnosis of the subject being diagnosed with multiple sclerosis established in accordance with the standard ICD-10-GM 2021, preferably M45.-, in accordance with the standard ICD-10-GM 2021.

The indication of the subject for which it is indicative to be at risk to develop the inflammatory disease, immunological disease and/or autoimmunological disease as mentioned in any of the embodiments described herein may be, preferably, is established by any method(s) and/or parameter(s) commonly used in the art and known to the person skilled in the art. More preferably, the indication is established in accordance with the ICD-10-GM 2021.

The indication of the subject for which it is indicative to be at risk to develop the inflammatory disease, immunological disease and/or autoimmunological disease may be, preferably, is established by any method(s) and/or parameter(s) commonly used in the art and known to the person skilled in the art, for instance family history and/or genetic association like HLA-B*27 in case of Bechterew's disease, rheumatoid arthritis, Morbus Crohn, reactive arthritis or juvenile arthritis.

Preferably, the subject for which it is indicative to be at risk to develop the inflammatory disease, immunological disease and/or autoimmunological disease, is a subject for which it is indicative to develop arthritis, monoarthritis, oligoarthritis, polyarthritis and/or any of the inflammatory rheumatic diseases listed above.

Preferably, the expression “treating” as mentioned in any of the embodiments described herein refers to a subject being diagnosed with the inflammatory disease, immunological disease and/or autoimmunological disease and the subject's condition is improved e.g. pain and/or inflammation are relieved or disappear; organ, tissue and/or joint destruction is prevented; blood parameters are improved or normalized; further progress of organ, tissue and/or joint destruction is slowed down, stopped and/or regeneration has occurred; organ, tissue and/or joint functional ability is preserved or improved; swelling of joints and/or pressure sensitivity is reduced; the subject's normal lifestyle maintained; and/or the subject's quality of life and/or mental and/or physical wellbeing are maintained or improved.

Preferably, the expression “preventing” as mentioned in any of the embodiments described herein refers to a subject being diagnosed to be at risk to develop the inflammatory disease, immunological disease and/or autoimmunological disease and the subject's healthy condition is maintained and the onset of the symptoms of the inflammatory disease, immunological disease and/or autoimmunological disease in the subject is prevented and/or delayed; organ, tissue and/or joint destruction is prevented; organ, tissue and/or joint functional ability is preserved; the subject's normal lifestyle is maintained; and/or the subject's quality of life and/or mental and/or physical wellbeing are maintained or improved.

The expression “PBMCs” (“peripheral blood mononuclear cells”) as used in any of the embodiments described herein typically refers to a sub-population of cells of the cellular blood components, particularly of the leucocytes. Preferably, leucocytes comprise, more preferably consist of as cellular components, lymphocytes and monocytes. Preferably, PBMCs comprise, preferably consist of as cellular components, lymphocytes and monocytes, whereas erythrocytes and platelets (which do not possess a nucleus) and granulocytes (which possess multi-lobed nuclei) are not present in effective amounts, essentially absent or absent, more preferably absent. More preferably, the PBMCs as mentioned in any of the embodiments described herein are lymphocytes, such as natural killer cells, B-cells and/or T-cells, more preferably B-cells and/or T-cells, even more preferably are naïve PBMCs, still more preferably naïve lymphocytes, still even more preferably naïve B-cells and/or naïve T-cells, and still even more preferably are naïve T-cells.

Preferably, the PBMCs as mentioned in any of the embodiments described herein are blood-derived PBMCs. This applies equally to PBMCs, which are administered e.g. injected (as it may be the case in step (A-8)), as well as to any accumulated PBMCs or PBMCs that may be accumulated. Hence, the expression “PBMCs” preferably does not refer to PBMCs inherently reside within tissues, preferably within the skin. Hence, PBMCs inherently residing within the skin, particularly the skin tissue, are preferably not encompassed in the accumulated PBMCs, in the accumulation of PBMCs or the PBMCs to be accumulated.

PBMCs may be extracted from the blood, preferably whole blood, and may then be present in an isolated, preferably purified, form, in the following termed “isolated PBMCs”, or they may be present as a sub-population of cells of the cellular blood components within the blood, preferably whole blood, in the following termed “blood PBMCs”. In isolated PBMCs, blood components and cellular blood components other than PBMCs are preferably not present in effective amounts, essentially absent or absent, more preferably absent. Isolated PBMCs may be obtained by any method known to the person skilled in the art, preferably by ficoll-extraction in combination with gradient centrifugation or by apheresis, more preferably by apheresis, using whole blood, preferably as described in the Materials and Methods' section ‘Preparation of PBMCs’.

The expression “lymphocytes” as mentioned in any of the embodiments described herein typically refers to a sub-population of cells of the PBMCs. Lymphocytes again may comprise sub-populations of cells, including B-cells, T-cells and/or natural killer cells. Preferably lymphocytes may comprise, preferably consist of as cellular components, B-cells including naïve and affected B-cells, like mature B-cells; natural killer cells; and/or T-cells including naïve and affected T-cells, like mature T-cells. More preferably, lymphocytes as mentioned in any of the embodiments described herein comprise, more preferably consist of as cellular components, naïve lymphocytes, affected lymphocytes and/or natural killer cells, even more preferably naïve lymphocytes, still even more preferably naïve B-cells and/or naïve T-cell, further preferably naïve T-cells. Preferably, as mentioned in any of the embodiments described herein, the lymphocytes are naïve lymphocytes, more preferably comprising, even more preferably consisting of as cellular components, naïve B-cells and/or naïve T-cells. Still even more preferably, the lymphocytes are naïve T-cells.

The expression “naïve” or “naïve cells” as mentioned in any of the embodiments described herein in respect to any type of cells like naïve PBMCs, naïve lymphocytes, naïve B-cells and/or naïve T-cells, typically have not been exposed to their corresponding antigen.

Preferably, such naïve cells are not activated and/or they have a potential to be affected by the immunomodulatory substance(s) and possibly dendritic cells, more preferably both. Upon affectation, naïve cells become affected cells. Even more preferably, naïve cells are not activated, have a potential to be affected and have not been exposed to their corresponding antigen. Typically, the expression “naïve” does not exclude that the naïve cells have already gained a certain degree of regulation, induction, pre-activation, activation, suppression, maturation, differentiation, modulation, proliferation, migration, survival, changed gene expression, preferably as long as such cells still have a potential to be affected by the immunomodulatory substance(s) and possibly dendritic cells, by at least one member of the list of possible affectations, like attraction, migration, survival, changed gene expression, cell-cell interaction, regulation, induction, suppression, maturation, differentiation, modulation and/or proliferation.

The expression “the potential to be affected” as mentioned in any of the embodiments described herein in respect to any type of cells like LHs, peripheral T_{reg}s, naïve PBMCs, naïve lymphocytes, naïve B-cells and/or naïve T-cells typically means that such LHs, peripheral T_{reg}s or naïve cells are upon affectation caused by the immunomodulatory substance(s) attracted, regulated, induced, pre-activated, activated, suppressed, matured, differentiated, modulated, proliferated, migrate, survive, have changed gene expression, process antigen and/or undergo cell-cell interaction, more preferably attracted, matured, pre-activated, activated, differentiated, proliferated, migrate, survive, have changed gene expression, process antigen and/or undergo cell-cell interaction.

The expression “affected” or “affected cells” as mentioned in any of the embodiments described herein in respect to any type of cells like affected LHs, affected peripheral T_{reg}s or affected PBMCs, affected lymphocytes, affected B-cells and/or affected T-cells typically means that such affected cells have, due to the affectation caused by the immunomodulatory substance(s) undergone attraction, migration, survival, changed gene expression, processing of antigen, cell-cell interaction, regulation, induction, pre-activation, activation, suppression, maturation, differentiation, modulation and/or proliferation, more preferably undergone attraction, migration, survival, changed gene expression, processing of antigen, cell-cell interaction, maturation, pre-activation, activation, differentiation and/or proliferation, even more preferably undergone migration, survival, changed gene expression, processing of antigen, cell-cell interaction, maturation, pre-activation, activation, differentiation and/or proliferation. Preferably, “affected” excludes that such affected cells have undergone activation due to the affectation caused by the immunomodulatory substance(s) and possibly dendritic cells, i.e. such affected cells do not provide an increased aggression against antigen, autoantigen and/or antigen presenting cells.

The expression “attract”, “attracting”, “attracted” and/or “attraction” as mentioned in any of the embodiments described herein in respect to any type of cells like LHs, peripheral T_{reg}s, PBMCs, lymphocytes, B-cells and/or T-cells, particularly naïve PBMCs, naïve lymphocytes, naïve B-cells and/or naïve T-cells typically means that such cells are caused to directionally migrate towards an attractor. Such attractor may be, but is not necessarily, e.g. the immunomodulatory substance(s). The expression for instance means that e.g. PBMCs may be caused to leave the lumen of the skin capillaries by crossing the vascular endothelial and enter into the surrounding skin tissue towards the site where the immunomodulatory substance(s) is present. As already stated in further detail above, this migration mechanism is known to naturally occur and does not require further action or affectation. Nevertheless, such an attractor may facilitate or contribute to such migration by providing a beneficial micro milieu. Therefore, the affectation may, but not necessarily, be amongst others an attraction. This could be advantageous in case of e.g. steps (A-0) to (A-7), where the accumulation of PBMCs is believed to be generated particularly within the lumen of the capillaries of the skin. The crossing of the PBMCs of the capillary wall then relies on the naturally occurring process and is believed to lead to an accumulation within the skin, particularly within the skin tissue. Contrary, in case of e.g. step (A-8), the PBMCs may be administered by injection and the accumulation of PBMCs is directly generated within the skin, particularly within the skin tissue. A migration into the skin tissue is then not required. In any case, such accumulated PBMCs are believed to be trapped and hold within the skin. The LHs and peripheral T_{reg}s already reside within the skin. Furthermore, the immunomodulatory substance(s) administered are also believed to be trapped and hold within the skin. Thereby an in-vivo incubation of the LHs, peripheral T_{reg}s and/or PBMCs with the immunomodulatory substance(s) is provided so that the LHs, peripheral T_{reg}s and/or PBMCs may become affected LHs, affected peripheral T_{reg}s and/or affected PBMCs. These affected LHs, affected peripheral T_{reg}s and/or affected PBMCs are preferably attracted, regulated, induced, pre-activated, activated, suppressed, matured, differentiated, modulated, proliferated and/or migrate, survive, have changed gene expression, process antigen and/or undergo cell-cell interaction.

The expressions “proliferate”, “proliferating”, “proliferated” and/or “proliferation as mentioned in any of the embodiments described herein in respect to any type of cells typically means that the amounts of such cells, relative to the amounts of such cells prior to affectation caused by the immunomodulatory substance(s), increase/are increased or decrease/are decreased, preferably increase/are increased (it may be noted that in dependency of the type of cell, a certain amount of such cells may already be present prior to affectation due to immune processes naturally occurring in the subject's body). For instance, in case regulatory T-cells and/or helper T-cells, or a subset thereof, are proliferated, this means that the amounts of such cells are increased relative to the amounts prior to affectation, preferably within the blood of the subject (it may be noted that a certain amount of such cells may already be present prior to affectation due to immune processes naturally occurring in the subject's body).

The expressions “differentiate”, “differentiating”, “differentiated” and/or “differentiation” as mentioned in any of the embodiments described herein in respect to any type of cells typically means that such cells may already be differentiated to a certain degree and they do have the potential to further differentiate and/or mature.

The expressions “mature”, “maturing”, “matured” and/or “maturation” as mentioned in any of the embodiments described herein in respect to any type of cells typically means that such cells do not have the potential to further differentiate and/or mature.

Differentiated cells may for instance be helper T-cells, or subsets thereof, having the potential to mature to for instance regulatory T-cells. Mature cells may for instance be regulatory T-cells which have no longer the potential for a further differentiation and/or maturation.

The expression “affected LHs” as mentioned in any of the embodiments described herein typically refers to LHs that have, without wishing to be bound to theory, due to the affectation caused by the immunomodulatory substance(s), undergone attraction, migration, survival, changed gene expression, processing of antigen, cell-cell interaction, regulation, induction, pre-activation, activation, maturation, differentiation, modulation and/or proliferation, more preferably undergone migration, changed gene expression, processing of antigen, cell-cell interaction, regulation, induction, pre-activation, activation, maturation, differentiation and/or modulation.

The expression “affected peripheral T_{reg}s” as mentioned in any of the embodiments described herein typically refers to peripheral T_{reg}s that have, without wishing to be bound to theory, due to the affectation caused by the immunomodulatory substance(s), undergone attraction, migration, survival, changed gene expression, processing of antigen, cell-cell interaction, regulation, induction, pre-activation, activation, maturation, differentiation, modulation and/or proliferation, more preferably undergone attraction, migration, survival, changed gene expression, cell-cell interaction, regulation, induction, modulation and/or proliferation.

The expression “naïve PBMCs” as mentioned in any of the embodiments described herein typically refers to PBMCs that have differentiated, at least to a certain degree.

Preferably, naïve PBMCs as mentioned in any of the embodiments described herein have not been exposed to their corresponding antigen.

Preferably, naïve PBMCs are not activated and/or they have a potential to be affected by the immunomodulatory substance(s) and possibly dendritic cells, more preferably both. Upon affectation naïve PBMCs become affected PBMCs.

Even more preferably, naïve PBMCs are not activated, have a potential to be affected and have not been exposed to their corresponding antigen.

Preferably, all PBMCs, more preferably all lymphocytes, that leave the pivotal (central) lymphoid organs like the thymus or bone marrow are considered naïve PBMCs. More preferably, those leaving the bone marrow are naïve B-lymphocytes, also called naïve B-cells, and/or those leaving the thymus are naïve T-lymphocytes, also called naïve T-cells.

Naïve PBMCs as mentioned in any of the embodiments described herein more preferably comprise, even more preferably consist of as cellular components, naïve lymphocytes, even more preferably naïve T-cells and/or naïve B-cell, still more preferably naïve T-cells.

The expression “affected PBMCs” as mentioned in any of the embodiments described herein typically refers to naïve PBMCs upon affectation caused by the immunomodulatory substance(s) and possibly dendritic cells. Without wishing to be bound to theory, it is believed that affected PBMCs preferably comprise, more preferably consist of as cellular components, affected lymphocytes, even more preferably affected B-cells and/or affected T-cells, still more preferably affected T-cells, regulatory B-cells, helper T-cells or a subset thereof, and/or regulatory T-cells; even more preferably regulatory B-cells, helper T-cells or a subset thereof, and/or regulatory T-cells; still more preferably helper T-cells, or a subset thereof, and/or regulatory T-cells. Further, without wishing to be bound to theory, preferably, affected PBMCs have, due to the affectation caused by the immunomodulatory substance(s) and possibly dendritic cells, undergone attraction, migration, survival, changed gene expression, cell-cell interaction, regulation, induction, suppression, maturation, differentiation, modulation and/or proliferation, more preferably undergone attraction, migration, survival, changed gene expression, cell-cell interaction, maturation, differentiation, and/or proliferation, even more preferably undergone maturation, differentiation, and/or proliferation. Preferably, the expression “affected PBMCs” excludes that affected PBMCs have undergone activation due to the affectation caused by the immunomodulatory substance(s) and possibly dendritic cells. Hence, preferably, affected PBMCs do not provide an increased aggression against antigen, autoantigen and/or antigen presenting cells.

The expression “naïve lymphocytes” as mentioned in any of the embodiments described herein typically refers to lymphocytes that have differentiated, at least to a certain degree.

Preferably, naïve lymphocytes as mentioned in any of the embodiments described herein have not been exposed to their corresponding antigen.

Preferably, naïve lymphocytes are not activated and/or they have a potential to be affected by the immunomodulatory substance(s) and possibly dendritic cells, more preferably both. Upon affectation naïve lymphocytes become affected lymphocytes.

Even more preferably, naïve lymphocytes are not activated, have a potential to be affected and have not been exposed to their corresponding antigen.

Preferably, all lymphocytes that leave the pivotal (central) lymphoid organs like the thymus or bone marrow are considered naïve lymphocytes. More preferably, those leaving the bone marrow are naïve B-lymphocytes, also called naïve B-cells, and/or those leaving the thymus are naïve T-lymphocytes, also called naïve T-cells.

Naïve lymphocytes as mentioned in any of the embodiments described herein more preferably comprise, even more preferably consist of as cellular components, naïve T-cells and/or naïve B-cell, still more preferably naïve T-cells.

The expression “affected lymphocytes” as mentioned in any of the embodiments described herein typically refers to naïve lymphocytes upon affectation caused by the immunomodulatory substance(s) and possibly dendritic cells. Without wishing to be bound to theory, it is believed that affected lymphocytes, preferably comprise, more preferably consist of as cellular components, memory B-cells, plasma cells, regulatory B-cells, helper T-cells or a subset thereof, and/or regulatory T-cells; more preferably regulatory B-cells, helper T-cells or a subset thereof, and/or regulatory T-cells; even more preferably helper T-cells or a subset thereof, and/or regulatory T-cells. Further, without wishing to be bound to theory, preferably, affected lymphocytes have, due to the affectation caused by the immunomodulatory substance(s) and possibly dendritic cells, undergone attraction, migration, survival, changed gene expression, cell-cell interaction, regulation, induction, suppression, maturation, differentiation, modulation and/or proliferation, more preferably undergone attraction, migration, survival, changed gene expression, cell-cell interaction, maturation, differentiation, and/or proliferation, even more preferably undergone maturation, differentiation, and/or proliferation. Preferably, the expression “affected lymphocytes” excludes that affected lymphocytes have undergone activation due to the affectation caused by the immunomodulatory substance(s) and possibly dendritic cells. Hence, preferably, affected lymphocytes do not provide an increased aggression against antigen, autoantigen and/or antigen presenting cells.

The expression “naïve T-cells” as mentioned in any of the embodiments described herein typically refers to T-cells that have differentiated, preferably at least to a certain degree, in the thymus, and successfully undergone the positive and negative processes of central selection in the thymus. Furthermore, they may have or have been released by the thymus.

Preferably, naïve T-cells as mentioned in any of the embodiments described herein have not been exposed to their corresponding antigen.

Preferably, naïve T-cells are not activated and/or they have a potential to be affected by the immunomodulatory substance(s) and possibly dendritic cells, more preferably both. Upon affectation, naïve T-cells become affected T-cells.

Even more preferably, naïve T-cells are not activated, have a potential to be affected and have not been exposed to their corresponding antigen.

The expression “affected T-cells” as mentioned in any of the embodiments described herein typically refers to naïve T-cells upon affectation caused by the immunomodulatory substance(s) and possibly dendritic cells. Without wishing to be bound to theory, it is believed that affected T-cells preferably comprise, more preferably consist of as cellular components, helper T-cells and/or regulatory T-cells, more preferably helper T-cells and/or regulatory T-cells. Further, without wishing to be bound to theory, preferably, affected T-cells have, due to the affectation caused by the immunomodulatory substance(s) and possibly dendritic cells, undergone attraction, migration, survival, changed gene expression, cell-cell interaction, regulation, induction, suppression, maturation, differentiation, modulation and/or proliferation, more preferably undergone attraction, migration, survival, changed gene expression, cell-cell interaction, maturation, differentiation, and/or proliferation, even more preferably undergone maturation, differentiation, and/or proliferation. Preferably, the expression “affected T-cells” excludes that affected T-cells have undergone activation, particularly not due to the affectation caused by the immunomodulatory substance(s) and possibly dendritic cells. Hence, preferably, affected T-cells do not provide an increased aggression against antigen, autoantigen and/or antigen presenting cells.

It may be pointed out that according to the above definitions of naïve T-cells and affected T-cells, the helper T-cells can be considered as naïve T-cells and/or as affected T-cell.

The expression “naïve B-cells” as mentioned in any of the embodiments described herein typically refers to B-cells that have differentiated, at least to a certain degree, in the bone marrow.

Preferably, naïve B-cells as mentioned in any of the embodiments described herein have not been exposed to their corresponding antigen.

Preferably, naïve B-cells are not activated and/or they have a potential to be affected by the immunomodulatory substance(s) and possibly dendritic cells, more preferably both. Upon affectation, naïve B-cells become affected B-cells.

Even more preferably, naïve B-cells are not activated, have a potential to be affected and have not been exposed to their corresponding antigen.

The expression “affected B-cells” as mentioned in any of the embodiments described herein typically refers to naïve B-cells upon affectation caused by the immunomodulatory substance(s) and possibly dendritic cells. Without wishing to be bound to theory, it is believed that affected B-cells preferably comprise, more preferably consist of as cellular components, memory B-cells, plasma cells and/or regulatory B-cells, even more preferably regulatory B-cells. Preferably, affected B-cells have, due to the affectation caused by the immunomodulatory substance(s) and possibly dendritic cells, undergone attraction, migration, survival, changed gene expression, cell-cell interaction, regulation, induction, suppression, maturation, differentiation, modulation and/or proliferation, more preferably undergone attraction, migration, survival, changed gene expression, cell-cell interaction, maturation, differentiation, and/or proliferation, even more preferably undergone maturation, differentiation, and/or proliferation. Preferably, the expression “affected B-cells” excludes that affected B-cells have undergone activation due to the affectation caused by the immunomodulatory substance(s) and possibly dendritic cells. Hence, preferably, affected B-cells do not provide an increased aggression against antigen, autoantigen and/or antigen presenting cells.

If not mentioned otherwise, the definitions of LHs, peripheral T_{reg}s, affected LHs, affected peripheral T_{reg}s, PBMCs, naïve PBMCs, affected PBMCs, lymphocytes, naïve lymphocytes, affected lymphocytes, T-cells, naïve T-cells, affected T-cells, helper T-cells or a subset thereof, B-cells, naïve B-cells, affected B-cells and the preferred embodiments thereof apply generally to any of the embodiments as described herein in which PBMCs, naïve PBMCs, affected PBMCs, lymphocytes, naïve lymphocytes, affected lymphocytes, T-cells, naïve T-cells, affected T-cells, helper T-cells or a subset thereof, B-cells, naïve B-cells and/or affected B-cells are mentioned.

If not mentioned otherwise, it is to be understood that any embodiment or definition of the immunomodulatory substance(s) as described herein is independently and mutatis mutandis applicable to the immunomodulatory substance(s) as mentioned in any of the embodiments described herein, particularly the immunomodulatory substance(s) for use according to the present invention, the immunomodulatory substance(s) of steps (B), (B₁) and/or step (C), the pharmaceutical composition, injectable dosage form, topical dosage forms, medical devices and/or kits of parts.

The expression “immunomodulatory substance” or “immunomodulatory substance(s)” as mentioned in any of the embodiments described herein, specifically the immunomodulatory substance(s) for use according to the present invention and/or the immunomodulatory substance(s) of steps (B), (B₁) and/or (C) of the method, the pharmaceutical compositions, injectable dosage forms, topical dosage forms, medical devices and/or kits of parts, may be, preferably is, any type of substance(s) capable of affecting and/or which affect(s), preferably directly and/or indirectly, the LHs, peripheral T_{reg}s and/or PBMCs.

The expression “to affect” or “affecting” as mentioned in any of the embodiments described herein in respect to the immunomodulatory substance(s) and in any of the preferred embodiments thereof typically means that the immunomodulatory substance(s) attracts, regulates, induces, pre-activates, activates, suppresses, matures, differentiates, modulates, proliferates, promotes migration, survival, changed gene expression, processing of antigen, cell-cell interaction LHs, peripheral T_{reg}s and/or PBMCs.

Preferably, the immunomodulatory substance(s) regulates, induces, pre-activates, activates, matures, differentiates, modulates, proliferates, causes migration, survival, changed gene expression, processing of antigen and/or cell-cell interaction, more preferably regulates, induces, pre-activates, activates, matures, differentiates, modulates, promotes migration, changed gene expression, processing of antigen and/or cell-cell interaction the LHs.

Preferably the immunomodulatory substance(s) attracts, regulates, induces, pre-activates, activates, matures, differentiates, modulates, proliferates, migrates, causes survival, changed gene expression, cell-cell interaction, more preferably attracts, regulates, induces, modulates, proliferates, promotes migration, survival, changed gene expression and/or cell-cell interaction the peripheral T_{reg}s.

Preferably, the immunomodulatory substance(s) attracts, matures, differentiates and/or proliferates PBMCs, even more preferably matures, differentiates and/or proliferates PBMCs. In other words the immunomodulatory substance(s) may attracts, regulates, induces, modulates and/or suppresses the PBMCs and/or causes the PBMCs to mature, differentiate and/or to proliferate. Preferably, “to affect” or “affecting” in respect of PBMCs excludes that the immunomodulatory substance(s) activates PBMCs. Hence, preferably, upon affectation, affected PBMCs do not provide an increased aggression against antigen, autoantigen and/or antigen presenting cells. More preferably, the PBMCs are naïve PBMCs, even more preferably lymphocytes, still more preferably T-cells and/or B-cells, still even more preferably naïve lymphocytes, further preferably naïve B-cells and/or naïve T-cells, further preferably naïve T-cells.

The expression “capable of affecting” as mentioned in any of the embodiments described herein in respect to the immunomodulatory substance(s) and in any of the preferred embodiments thereof typically means that the immunomodulatory substance(s) is capable of attracting, regulating, inducing, pre-activating, activating, suppressing, maturing, differentiating, modulating, proliferating, promoting migration, survival, changed gene expression, processing of antigen and/or cell-cell interaction of LHs, peripheral T_{reg}s and/or PBMCs. Hence, the immunomodulatory substance(s) may be capable of affecting and/or affects the LHs, peripheral T_{reg}s and/or PBMCs by attracting, regulating, inducing, pre-activating, activating, suppressing, maturing, differentiating, modulating, proliferating, promoting migration, survival, changed gene expression, processing of antigen and/or cell-cell interaction.

Preferably the immunomodulatory substance(s) is capable of regulating, inducing, pre-activating, activates, maturing, differentiating, modulating, proliferating, causing migration, survival, changed gene expression, processing of antigen and/or cell-cell interaction, more preferably regulating, inducing, pre-activating, activating, maturing, differentiating, modulating, promoting migration, changed gene expression, processing of antigen and/or cell-cell interaction the LHs. Hence, in other words the immunomodulatory substance(s) may more preferably be capable of affecting and/or affects the LHs by regulating, inducing, pre-activating, activates, maturing, differentiating, modulating, proliferating, causing migration, survival, changed gene expression, processing of antigen and/or cell-cell interaction, more preferably regulating, inducing, pre-activating, activating, maturing, differentiating, modulating, promoting migration, changed gene expression, processing of antigen and/or cell-cell interaction.

Preferably the immunomodulatory substance(s) is capable of attracting, regulating, inducing, pre-activating, activating, maturing, differentiating, modulating, proliferating, migrating, causing survival, changed gene expression, cell-cell interaction, more preferably attracts, regulating, inducing, modulating, proliferating, promoting migration, survival, changed gene expression and/or cell-cell interaction the peripheral T_{reg}s. Hence, in other words the immunomodulatory substance(s) may more preferably be capable of affecting and/or affects the peripheral T_{reg}s attracting, regulating, inducing, pre-activating, activating, maturing, differentiating, modulating, proliferating, migrating, causing survival, changed gene expression, cell-cell interaction, more preferably attracts, regulating, inducing, modulating, proliferating, promoting migration, survival, changed gene expression and/or cell-cell interaction.

Preferably the immunomodulatory substance(s) is capable of attracting, maturing, differentiating and/or proliferating PBMCs, even more preferably maturing, differentiating and/or proliferating PBMCs. Preferably, “capable of affecting” in respect of PBMCs excludes that the immunomodulatory substance(s) is capable of activating PBMCs. Hence, preferably, upon a potential affectation, affected PBMCs do not provide an increased aggression against antigen, autoantigen and/or antigen presenting cells. More preferably, the PBMCs are naïve PBMCs, even more preferably lymphocytes, still more preferably T-cells and/or B-cells, still even more preferably naïve lymphocytes, further preferably naïve B-cells and/or naïve T-cells, further preferably naïve T-cells.

Hence, more preferably, the immunomodulatory substance(s) is capable of affecting and/or affects the LHs, peripheral T_{reg}s or PBMCs by attracting, proliferating, differentiating and/or maturing, even more preferably by proliferating, differentiating and/or maturing, even more preferably differentiating and/or maturing. Hence, more preferably, the immunomodulatory substance(s) is capable of attracting, proliferating, differentiating and/or maturing the PBMCs and/or attracts, proliferates, differentiates and/or matures the PBMCs. Even more preferably, the immunomodulatory substance(s) is capable of proliferating, differentiating and/or maturing the PBMCs and/or proliferates, differentiates and/or matures the PBMCs. Still even more preferably, the immunomodulatory substance(s) is capable of differentiating and/or maturing the PBMCs and/or differentiates and/or matures the PBMCs. More preferably, the PBMCs are naïve PBMCs, even more preferably lymphocytes, still more preferably T-cells and/or B-cells, still even more preferably naïve lymphocytes, further preferably naïve B-cells and/or naïve T-cells, even further preferably naïve T-cells.

The expression “directly” as mentioned in any of the embodiments described herein typically means in the context of ‘affecting’ or ‘capable of affecting’ LHs, peripheral T_{reg}s and/or PBMCs that the immunomodulatory substance(s) acts or is capable of acting by itself as the effector causing a desired affectation of the LHs, peripheral T_{reg}s and/or PBMCs. For instance it may activate of may be capable of activating the respective receptor of the immunomodulatory substance(s) of a cell. The receptor of the immunomodulatory substance(s) may for be for instance one of the LHs, peripheral T_{reg}s and/or PBMCs. For instance in case the immunomodulatory substance(s) is a cytokine(s), the cytokine(s) may be capable of activating the respective cytokine-receptor(s). More preferably, the PBMCs are naïve PBMCs, even more preferably lymphocytes, still more preferably T-cells and/or B-cells, still even more preferably naïve lymphocytes, further preferably naïve B-cells and/or naïve T-cells, further preferably naïve T-cells.

The expression “indirectly” as mentioned in any of the embodiments described herein typically means in the context of ‘affecting’ or ‘capable of affecting’ LHs, peripheral T_{reg}s and/or PBMCs that the immunomodulatory substance(s) is any substance(s) that results in or causes or is capable of resulting in or causing the generation of the immunomodulatory substance(s), such as in particular a precursor, propeptide or prodrug. In other words, the immunomodulatory substance(s) may encompass substances which are usually not active as such, i.e. have no immunomodulatory activity like the affectation or capability of affectation, but which, upon use in accordance with the present invention, are converted into the actually active immunomodulatory substance. Thereby the desired affectation of the LHs, peripheral T_{reg}s and/or PBMCs is indirectly effected. This may for instance be the case if the immunomodulatory substance(s) is any substance(s) like:
an inductor inducing cells to produce and/or secrete the immunomodulatory substance(s), wherein such cells may be any cells capable of producing and/or secreting the immunomodulatory substance(s).
a precursor of the immunomodulatory substance(s) which e.g. is metabolized by the subject's body to generate the immunomodulatory substance(s). The precursor of the immunomodulatory substance(s) includes, beside others, for instance peptide(s), protein(s), protein-analogue(s), protein-variant(s), propeptide(s), derivative(s) thereof, RNA, DNA, salts, capped immunomodulatory substance(s), etc.;
an inhibitor for a substance, enzyme or factor preventing the expression, interaction, production, secretion and/or activity of the immunomodulatory substance(s), like for instance particles or nanoparticles or a cap-structure. The immunomodulatory substance(s) may for instance be packed in particles for delivery. These particles may sterically prevent and protect the substance from interacting with its target, e.g. the receptor, and the substance may for instance exert its action only after the release from the particles in the patient's body;
a prodrug of the immunomodulatory substance(s);
and/or
a co-drug of the immunomodulatory substance(s).

More preferably, the PBMCs to be affected are naïve PBMCs, even more preferably lymphocytes, still more preferably T-cells and/or B-cells, still even more preferably naïve lymphocytes, further preferably naïve B-cells and/or naïve T-cells, further preferably naïve T-cells; etc.

The expression “biopharmaceutical” encompasses biologics, biosimilars, biomimics, biobetters and/or biosuperiors.

More preferably, the immunomodulatory substance(s) as mentioned in any of the embodiments described herein, specifically the immunomodulatory substance(s) for use according to the present invention and/or the immunomodulatory substance(s) of steps (B), (B₁) and/or (C) of the method, the pharmaceutical compositions, injectable dosage forms, topical dosage forms, medical devices and/or kits of parts:
is capable of affecting and/or affects, preferably directly and/or indirectly, the LHs, peripheral T_{reg}s and/or PBMCs in the subject, more preferably within the skin of the subject;
is capable of affecting and/or affects, preferably directly and/or indirectly, the LHs, peripheral T_{reg}s and/or PBMCs in a vertebrate, preferably a mammal, more preferably a human or a mammal animal as defined above, more preferably in the subject as defined in any of the embodiments according to the present invention, more preferably within the skin of a vertebrate, even more preferably within the skin of a mammal, still more preferably within the skin of a human or a mammal animal as defined above, further preferably within the skin of the subject;
is capable of affecting and/or affects, preferably directly and/or indirectly, the LHs, peripheral T_{reg}s and/or PBMCs within the skin of the subject;
even more preferably is capable of directly affecting and/or directly affects the LHs, peripheral T_{reg}s and/or PBMCs in the subject, more preferably within the skin of the subject.

Preferably, the PBMCs are naïve PBMCs, more preferably lymphocytes, even more preferably T-cells and/or B-cells, still more preferably naïve lymphocytes, still even more preferably naïve B-cells and/or naïve T-cells, further preferably naïve T-cells.

The immunomodulatory substance(s) as mentioned in any of the embodiments described herein, specifically the immunomodulatory substance(s) for use according to the present invention and/or the immunomodulatory substance(s) of steps (B), (B₁) and/or (C) of the method, the pharmaceutical compositions, injectable dosage forms, topical dosage forms, medical devices and/or kits of parts may encompass precursors, prodrugs and propeptides of such immunomodulatory substance(s) which are usually not active as such, i.e. have no immunomodulatory activity, but which, upon use in accordance with the present invention, are converted into the actually active immunomodulatory substance.

Hence, the immunomodulatory substance(s) may be any substance, molecule, peptide, protein, protein-analogue, protein-variant, biopharmaceutical, inductor, precursor, prodrug, mutein, co-drug, propeptide(s) and/or any derivative, fragment, pharmaceutically acceptable salt of any of these. More preferably it is any peptide, protein, protein-analogue, protein-variant, inductor, precursor, prodrug, mutein, co-drug and/or any derivative, fragment, pharmaceutically acceptable salt of any of these.

The immunomodulatory substance(s) in any of the embodiments described herein may be any naturally occurring or artificial immunomodulatory substance(s), as long as it has a sufficient biologic activity, i.e. an effective cross-reactivity in the subject, particularly is capable of activating the respective interferon-receptor(s).

The immunomodulatory substance(s) as mentioned in any of the embodiments described herein, specifically the immunomodulatory substance(s) for use according to the present invention and/or the immunomodulatory substance(s) of steps (B), (B₁) and/or (C) of the method, the pharmaceutical compositions, injectable dosage forms, topical dosage forms, medical devices and/or kits of parts is any peptide(s), protein(s) and/or any derivative(s) or fragment(s) of these, may be, preferably is similar or identical, more preferably identical, to any peptide, protein or any fragment of these naturally occurring in a vertebrate, preferably a human or a mammal animal as defined above, more preferably in the subject as defined in any of the embodiments according to the present invention.

The immunomodulatory substance(s) may comprise, preferably consists of an amino acid sequence which has an amino acid sequence identity to any peptide, protein or any fragment of these naturally occurring in a vertebrate, preferably a mammal, more preferably a human or a mammal animal as defined above, even more preferably in the subject as defined in any of the embodiments according to the present invention. Preferably, the amino acid sequence identity is 70% or more, still more preferably of 85% or more, still even more preferably 90% or more, further preferably 95% or more, even further preferably 97% or more, still further preferably 98% or more, still even further preferably 99% or more and 100% or less. Preferably, the amino acid sequence identity includes substitutions, insertions and/or deletions of amino acid residues in respect to the naturally occurring peptide, protein or any fragment of these. Preferably, the total number of substitutions, insertions and/or deletions or the total number combined of substitutions, insertions and/or deletions of amino acid residues is 60 or less, more preferably 40 or less, even more preferably 20 or less, still more preferably 12 or less, still even more preferably 8 or less, further preferably 4 or less, even further preferably 2 or less and zero or more, still further preferably zero.

The immunomodulatory substance(s) may be identical to an endogenous immunomodulatory substance(s) of the same genus and/or species the subject belongs to or the amino acid sequence identity refers to an endogenous immunomodulatory substance(s) of the same genus and/or species the subject belongs to.

The expression “is identical” typically means in this particular context that the immunomodulatory substance(s) is in respect to its primary, secondary, tertiary and quaternary protein structure identical to an/the immunomodulatory substance(s) of the same genus and/or species the subject belongs to, more preferably, also in view of the modification pattern, particularly glycosylations.

The immunomodulatory substance(s) the immunomodulatory substance(s) as mentioned in any of the embodiments described herein, specifically the immunomodulatory substance(s) for use according to the present invention and/or the immunomodulatory substance(s) of steps (B), (B₁) and/or (C) of the method, the pharmaceutical compositions, injectable dosage forms, topical dosage forms, medical devices and/or kits of parts may be derived from an individual belonging to the same genus and/or species as the subject, or which is identical to an/the immunomodulatory substance(s) of the same genus and/or species the subject belongs to. For instance, in case the subject is: a human the immunomodulatory substance(s) is a human immunomodulatory substance(s); in case the subject is a cow the immunomodulatory substance(s) is a bovine immunomodulatory substance(s); in case the subject is a horse the immunomodulatory substance(s) is an equine immunomodulatory substance(s); in case the subject is a donkey the immunomodulatory substance(s) is a donkey immunomodulatory substance(s); in case the subject is an elephant the immunomodulatory substance(s) is an elephant immunomodulatory substance(s); in case the subject is a sheep the immunomodulatory substance(s) is a sheep immunomodulatory substance(s); in case the subject is a goat the immunomodulatory substance(s) is a goat immunomodulatory substance(s); in case the subject is a pig the immunomodulatory substance(s) is a porcine immunomodulatory substance(s); in case the subject is a rabbit the immunomodulatory substance(s) is a rabbit immunomodulatory substance(s); in case the subject is a mouse the immunomodulatory substance(s) is a mouse immunomodulatory substance(s); in case the subject is a rat the immunomodulatory substance(s) is a rat immunomodulatory substance(s); in case the subject is a camel the immunomodulatory substance(s) is a camel immunomodulatory substance(s); in case the subject is a dromedary the immunomodulatory substance(s) is a dromedary immunomodulatory substance(s); in case the subject is a lama the immunomodulatory substance(s) is a lama immunomodulatory substance(s); in case the subject is an alpaca the immunomodulatory substance(s) is an alpaca immunomodulatory substance(s); in case the subject is a dog the immunomodulatory substance(s) is a dog immunomodulatory substance(s); and/or in case the subject is a cat the immunomodulatory substance(s) is a cat immunomodulatory substance(s).

Preferably, the immunomodulatory substance(s) as mentioned in any of the embodiments described herein, specifically the immunomodulatory substance(s) for use according to the present invention and/or the immunomodulatory substance(s) of steps (B), (B₁) and/or (C) of the method, the pharmaceutical compositions, injectable dosage forms, topical dosage forms, medical devices and/or kits of parts, is not the subject's endogenous immunomodulatory substance(s). In other words, preferably the immunomodulatory substance(s) is not an immunomodulatory substance(s) isolated from the subject's body, i.e. from the individual subject to be treated or prevented.

Preferably, the immunomodulatory substance(s) as mentioned in any of the embodiments described herein, specifically the immunomodulatory substance(s) for use according to the present invention and/or the immunomodulatory substance(s) of steps (B), (B₁) and/or (C) of the method, the pharmaceutical compositions, injectable dosage forms, topical dosage forms, medical devices and/or kits of parts, may be, preferably, is a recombinant immunomodulatory substance(s) which may be which may be generated by any recombinant expression techniques known to the person skilled in the art, a chemically synthesized immunomodulatory substance(s) synthesized by any production techniques known to the person skilled in the art, an artificially produced immunomodulatory substance(s) produced by any production techniques known to the person skilled in the art, a naturally occurring immunomodulatory substance(s) obtained from natural sources like an animal or human, or any combination thereof. More preferably, the immunomodulatory substance(s) is a recombinant immunomodulatory substance(s), chemically synthesized immunomodulatory substance(s), artificially produced immunomodulatory substance(s) or any combination thereof, even more preferably a recombinant immunomodulatory substance(s).

Furthermore, the immunomodulatory substance(s) for use according to the present invention and/or the immunomodulatory substance(s) of steps (B), (B₁) and/or (C) of the method, the pharmaceutical compositions, injectable dosage forms, topical dosage forms, medical devices and/or kits of parts may or may not be derivatized, stabilized, fused with other proteins or peptides, conjugated with polymers, contain amino acid analogue(s) or artificial amino acids, modified, covalently or non-covalently, for instance glycosylated or methylated by e.g. posttranslational modification, and/or oligomerized, e.g. dimerized or trimerized, an protein-analogue, protein-variant as long as it is capable of affecting and/or affects LHs, peripheral T_{reg}s and/or PBMCs. More preferably, the modification pattern and/or oligomerization status is similar or identical to the peptide, protein or any fragment of these naturally occurring in a vertebrate, preferably a mammal, more preferably a human or a mammal animal as defined above, even more preferably the subject as defined in any of the embodiments according to the present invention.

An advantage of such similarity or identity to immunomodulatory substance(s) naturally occurring in the subject's body, particularly in case of an identity, is that the raise of an immune response against such substances and elimination thereof by the subject's immune system cannot be expected (e.g. generation of antibodies). This appears particularly true in case the immunomodulatory substance(s) is vital for the subject. This for instance is the case if the immunomodulatory substance(s) is a cytokine(s), particularly IFN-γ (interferon gamma), IL-2 (interleukin 2), IL-4 (interleukin 4) or BDNF (brain-derived neurotropic factor). An immune system's attack on such immunomodulatory substance(s) would be most likely life-threatening or even fatal and is hence, not expected. In case of e.g. biologics and biosimilars, like TNF-α inhibitors (tumor necrosis factor alpha inhibitors) or IL-6 inhibitors (interleukin-6 inhibitors) monoclonal antibodies, the situation is different. They are artificial substances which do not naturally occur in the subject's body. Sooner or later they are usually recognized by the subject's immune systems as foreign and will be neutralized. This possible, because an elimination thereof by the subject's immune systems has no life-threatening consequences on the homeostasis of the immune system. Precisely, as soon as suitable antibodies are formed, such antibodies will eliminate or neutralize e.g. the biologic or biosimilar. It consequently loses its effect, at least to some extent, and clinical resistance for a treatment with the biologic or biosimilar is observed in the subject. The biologic or biosimilar consequently becomes ineffective. Moreover, this loss of effect is typically permanent and last a subject's lifetime. Memory B-cells usually remain as a remnant of the immune response in the body for the rest of the subject's life. Thereby they repeatedly ensure elimination of the biologic or biosimilar for the rest of the subject's lifetime. Moreover, this can also not be reversed and a substitution is difficult. Hence, in this event, this biologic or biosimilar cannot be effectively applied again in this particular individual once such antibodies were generated. Contrary to e.g. biologics or biosimilars, such loss off effectiveness is not expected for the immunomodulatory substance(s) preferably used in the present invention due to the reasons explained above. An additional advantage of such similarity or identity to immunomodulatory substance(s) naturally occurring in the subject's body, particularly in case of an identity, is that less or no adverse side effects, like malaise and drug intolerance, are observed and are not to be expected. However, this frequently the case for e.g. biologics and biosimilars. The patients treated according to the present invention even feel stronger and more energetic, presumably because the inflammation draining the subject's body is repressed (e.g. indicated by decreasing CRP amounts (C-reactive protein), a decreasing value of the BASDAI (Bath Ankylosing Spondylitis Disease Activity Index) or a decreasing value of the HAQ (Health Assessment Questionnaire)). The therapy of the present invention does not add any adverse side effects or incompatibilities, even in long term use over more than a year.

Furthermore, in the treatment of inflammatory diseases glucocorticoids are frequently used. Since glucocorticoids have also a high similarity or are even identical with substances naturally occurring in the subject's body, the following shall be mentioned. In conventional therapies glucocorticoids need to be administered in high concentrations to be effective. Due to that fact, in long-term use they develop severe adverse effects, in adults and even more in children. It is a general interest in medicine to administer drugs and pharmaceuticals in the lowest concentration still effective. However, with a lowering of the dosage of e.g. glucocorticoids it is not possible that they can confer their beneficial effects. Hence, in conventional therapies an unpleasant weighing between side effects and curative effects must be made.

Similarly, also the immunomodulatory substance(s), particularly e.g. IFN-γ and IL-2, are employed in conventional therapies or therapy attempts solely in very high dosages of immunomodulatory substance(s). Furthermore, it has been reported previously, that administering high concentrations of e.g. IFN-γ (50,000 ng recombinant IFN-γ) proved even to be no more effective than placebo in patients with rheumatoid arthritis (“A randomized double-blind study comparing twenty-four-week treatment with recombinant interferon-γ versus placebo in the treatment of rheumatoid arthritis”, Eric M. Veys M D, PhD, Charles-Joel Menkes M D, PhD, Paul Emery FRCP, First published: January 1997, https://doi.org/10.1002/art.1780400110).

In the present invention the highest preferred concentration of e.g. IFN-γ is 1,500 ng, which is 33-times less than used by Eric et al. In the Examples of the present invention an amount of even 5 ng IFN-γ has been effectively used, which is as much as 10,000-times less than the amount used by Eric et al.

Hence, the present invention is based on the further surprising finding that the immunomodulatory substance(s) can be administered in extremely low concentrations while still being effective.

Such low concentrations typically do not lead to a systemic increase of the immunomodulatory substance(s) in the subject's body, presumably at least not to a systemic increase which is effective (systemic effective increase). In other words, when scaling the administered doses of the immunomodulatory substance(s) to the entire body of the subject, the resulting systemic concentrations is assumed to remain in the magnitude of the endogenous levels. The systemic homeostasis of the immune system is unlikely to be altered.

The low amounts locally administered can easy be handled by the subject's metabolism. From these points of view adverse side effects are not to be expected even in long-term use and a weighing between side effects and curative effects is not be necessary.

Moreover, by administering low amounts of immunomodulatory substance(s), the effectiveness of the treatment, the reliability of the treatment success and the repeatability in a greater proportion of patients is even improved.

Without wishing to be bound to theory, it is believed that the tolerogenic phenotype of LHs finally leading to the generation of regulatory T-cells may only be promoted and fully executed by administering ultra-low amounts of immunomodulatory substance(s) like e.g. IFN-γ and IL-2.

Further, without wishing to be bound to theory, it is believed that by administering extremely low amounts of immunomodulatory substance(s), the end phase of an inflammatory reaction can be locally resembled e.g. within the skin. The generation of immunosuppressive T-cells like helper T-cells, or a subset thereof, and particularly regulatory T-cells is thereby promoted with at the same time avoiding the generation of highly aggressive pro-inflammatory cytotoxic T-cells. Thereby, the desired and therapeutic anti-inflammatory effects are promoted while the non-wanted and adverse pro-inflammatory effects are prevented. It is noted that cytotoxic T-cells are, in a sense, the pro-inflammatory counteractors of anti-inflammatory helper T-cells, or a subset thereof, and particularly regulatory T-cells.

However, when administering high amounts of immunomodulatory substance(s) it is believed that the desired anti-inflammatory effects may be cancelled out, not be achieved or even exceeded by the non-wanted pro-inflammatory effects. This could possibly be an explanation for why the high concentrations administered in Eric et al. do not show any significant beneficial effects beyond the placebo.

Precisely, in order to neutralize harmful antigen a healthy immune system produces during an inflammatory reaction large quantities of cytotoxic T-cells. As mentioned above, these cells are highly aggressive. The presence of high amounts of autoantigen in combination with immunomodulatory substance(s) such as IFN-γ is believed to cause naïve T-cells to develop into cytotoxic T-cells.

At the end of an inflammatory reaction all antigen is neutralized and there is an excess of cytotoxic T-cells, which are relieved of their task. To prevent these cells with their high aggression potential from causing damage elsewhere in the body, the immune system provides for e.g. regulatory T-cells. As stated before, such regulatory T-cells may act as immunosuppressive counterparts of cytotoxic

T-cells. They ensure that the extent of an immune activation is limited. At the end of an inflammatory response all antigen is neutralized, but immunomodulatory substance(s) like e.g. IFN-γ are still present. Consequently, at the end of an inflammatory response the milieu has turned towards the favour of regulatory T-cells and/or helper T-cells, or a subset thereof, respectively. Without the presence of high amounts of autoantigen in the simultaneous presence of immunomodulatory substance(s) such as IFN-γ, trailing or subsequent naïve T-cells no longer develop into cytotoxic T-cells—they will mature or develop into regulatory T-cells.

Consequently, without whishing to be bound to theory, the formation of regulatory T-cells is believed to be promoted by administering low amount of the immunomodulatory substance(s), either based on the theory relating to PBMCs or the the theory relating to LHs and peripheral T_{reg}s.

Without wishing to be bound to theory, it is believed that in the present invention in the skin, which is free from high amounts of antigen/autoantigen/allergen or essentially free of antigen/autoantigen/allergen, that is only low amounts of antigen/autoantigen/allergen may be present, formation of immunosuppressive T-cells like regulatory and/or helper T-cells is initiated. It is further believed that by accumulating PBMCs e.g. within the skin, and contacting them with immunomodulatory substance(s) by at the same time avoiding antigen, autoantigen or allergen contact in large amounts, immunosuppressive T-cells like regulatory T-cells and helper T-cells, or a subset thereof, are generated. It is further believed that by accumulating low amounts of autoantigen within the skin at the site where LHs and peripheral T_{reg}s reside at concomitant providance of immunomodulatory substance(s) by at the same time avoiding antigen, autoantigen or allergen contact in large amounts, immunosuppressive T-cells like regulatory T-cells and helper T-cells, or a subset thereof, are generated.

However, if high amounts of immunomodulatory substance(s) are administered, it is believed, without whishing to be bound thereto, that the LHs will develop to an immunogenic phenotyp instead of the tolerogenic, immunosuppressive phenotype. Furthermore, if high amounts of immunomodulatory substance(s) are administered, it is believed, again without whishing to be bound thereto, that a systemic increase of the concentration of immunomodulatory substance(s) is generated to an extent which is effective, i.e. a systemic effective increase is generated. Thereby, also e.g. rheumatically inflamed joints are flushed with immunomodulatory substance(s) such as IFN-γ in effective amounts while at the same time a large amount of autoantigen, i.e. joint tissue, is present. Consequently, cytotoxic T-cells may be generated within the joints. The autoagression within the joints may be even be increased. Without wishing to be bound to theory, the beneficial suppressive effects of e.g. the regulatory T-cells believed to the generated by the method of the present invention are cancelled or even exceeded to the worse.

In short, without wishing to be bound to theory, it is believed that naïve PBMCs, particularly naïve T-cells, are the targets of the present invention. By using e.g. the skin as an in-vivo incubator for the naïve T-cells, specifically and locally helper T-cells, or subsets thereof, and regulatory T-cells, may be produced. The latter are then considered to be the effectors which spread and exert their beneficial anti-inflammatory effect at locations of need. At the same time the generation of pro-inflammatory cytotoxic T-cells is elsewhere preferably in the subject's body avoided.

Hence, preferably, in any of the embodiments described herein it is intended to administer the immunomodulatory substance(s) in an amount low enough to avoid a systemic increase, particularly an effective systemic increase, of the concentration of the immunomodulatory substance(s) in the subject's body. Hence, the immunomodulatory substance(s) may be administered in an amount that, when scaled to the entire body of the subject, the resulting systemic concentrations remain in the magnitude of the endogenous levels. Thereby, without wishing to be bound to theory, it is believed that the generation of an increased concentration of the immunomodulatory substance(s) for instance at sites of inflammation like inflamed joint shall be avoided. As already stated above, for instance naïve T-cells can mature in the presence of antigen/autoantigen/allergen towards cytotoxic T-cells. These T-cells act pro-inflammatory thereby decreasing, cancelling or even reversing the beneficial anti-inflammatory effects conferred by the regulatory T-cells and/or helper T-cells, or a subset thereof. Hence, by administering the immunomodulatory substance(s) in sufficiently low amounts, it is believed to solely promote the generation of regulatory T-cells and/or helper T-cells, or a subset thereof, locally within the skin while the generation of cytotoxic T-cells elsewhere in the subject's body is prevented.

Preferably, in any of the embodiments described herein, particularly in step (B), (B₁) and/or (C), the immunomodulatory substance(s) is administered in an amount that does not cause a systemic increase, more preferably an effective systemic increase, of the concentration of immunomodulatory substance(s) in the subject, preferably the blood of the subject, and/or that causes a systemic increase of the concentration of the immunomodulatory substance(s). Preferably, immunomodulatory substance(s) is administered in an amount low enough that the systemic increase is non-effective.

Preferably, in any of the embodiments described herein, particularly in step (B), (B₁) and/or (C), the immunomodulatory substance(s) is administered in an amount that causes a local increase, preferably an effective local increase, of the concentration of the immunomodulatory substance(s) in the subject, preferably in the skin tissue of the subject. The skin tissue is preferred, because unlike administration into the blood, no or less flushing away and dilution of the immunomodulatory substance(s) takes place.

Preferably, in any of the embodiments described herein, particularly in step (B), (B₁) and/or (C), the generated increased concentration, preferably the generated effective increased concentration of the immunomodulatory substance(s) administered is only local but not systemic in the subject.

Preferably, in any of the embodiments described herein, particularly in step (B), (B₁) and/or (C), the immunomodulatory substance(s) is administered in an amount that does not cause a systemic activation, preferably an effective systemic activation, of the respective receptor of the immunomodulatory substance(s) of a cell, and/or that does not cause a systemic generation, preferably an effective systemic generation, of the immunomodulatory substance(s), preferably in the subject.

Preferably, in any of the embodiments described herein, particularly in step (B), (B₁) and/or (C), the immunomodulatory substance(s) is administered in an amount that causes a local activation, preferably an effective local activation of the respective receptor of the immunomodulatory substance(s) of a cell, and/or causes a local generation, preferably an effective local generation of the immunomodulatory substance(s), preferably in the subject. Preferably, the activation and/or generation, more preferably the effective generation and/or effective activation is only local but not systemic in the subject.

Preferably, the immunomodulatory substance(s) as mentioned in any of the embodiments described herein, specifically the immunomodulatory substance(s) for use according to the present invention and/or the immunomodulatory substance(s) of steps (B), (B₁) and/or (C) of the method, the pharmaceutical compositions, injectable dosage forms, topical dosage forms, medical devices and/or kits of parts, is a cytokine-like acting substance(s), preferably a cytokine(s), or any peptide, protein, protein-analogue, peptide, protein-variant, derivative, fragment, biopharmaceutical, inductor, precursor, prodrug, mutein, co-drug thereof and/or pharmaceutically acceptable salt of any of these.

In the present invention, the expression “cytokine-like acting substance”, “interferon-like acting substance”, “interleukin-like acting substance”, “neutrophin-like acting substance”, or any similar expression, designates substances which directly or indirectly, e.g. after being metabolized, exert an effect similar or identical to the effect exerted by the substance itself in the body of the subject, and encompasses in each case also the substance itself, i.e. the “cytokine-like acting substance” encompasses the cytokine, the “interferon-like acting substance” encompasses the interferon, the “interleukin-like acting substance” encompasses the interleukin, and the “neutrophin-like acting substance” encompasses the neutrophin, and so on.

This applies also where herein in the following terms like “cytokine and/or cytokine-like acting substance” or “cytokine or cytokine-like acting substance” or similar terms are used, i.e. also in these terms the expression “cytokine-like acting substance” designates the cytokine itself and any substances which directly or indirectly, e.g. after being metabolized, exert an effect similar or identical to the effect exerted by the cytokine itself in the body of the subject.

The expression “cytokine-like acting substance” or “cytokine-like acting substance(s)” as mentioned in any of the embodiments described herein can be, preferably is, any substance that exerts a similar or identical effect to a cytokine in the body of the subject when administered thereto. The cytokine-like acting substance(s) may encompass precursors, prodrugs and propeptides of such substances which are usually not active as such, i.e. have no immunomodulatory activity, but which, upon use in accordance with the present invention, are converted into the actually active cytokine-like acting substance(s). Hence, it may be any substance, molecule, peptide, protein protein-analogue, protein-variant, derivative, fragment, biopharmaceutical, inductor, precursor, prodrug, mutein, co-drug and/or any derivative, fragment and/or pharmaceutically acceptable salt of any of these. Therefore cytokine-like acting substance(s) may:
activates or is/are capable of activating a respective cytokine-receptor(s) of a cell. The cytokine-receptor(s) may for instance be the receptor of the LHs, peripheral T_{reg}s or PBMCs; and/or
results in or causes or is capable of resulting in or causing the generation of a cytokine(s) and/or a cytokine-like acting substance(s).

Preferably, the cytokine-like acting substance(s) activates or is capable of activating the respective cytokine-receptor(s). The activating or the capability of activating the respective cytokine-receptor(s) may be directly and/or it may be indirectly.

Preferably, the cytokine-like acting substance(s) activates or is/are capable of activating a respective cytokine-receptor(s) within the skin of the subject. The cytokine-like acting substance(s) may be any naturally occurring or artificial cytokine-like acting substance(s) as long as it has a sufficient biologic activity, i.e. an effective cross-reactivity in the subject, particularly is capable of activating the respective cytokine-receptor(s). Preferably, the cytokine-like acting substance(s) is known to the person skilled in the art at the effective date of filing of the present invention.

Preferably, the cytokine-like acting substance is any peptide, protein, protein-analogue, protein-variant) or any derivative, fragment, biopharmaceutical, inductor, precursor, prodrug, mutein, co-drug and/or pharmaceutically acceptable salt of any of these.

In a preferred embodiment, the cytokine-like acting substance(s) may be as detailed below amongst others a cytokine(s) and/or any peptide, protein, protein-analogue, peptide, protein-variant, derivative, fragment, biopharmaceutical, inductor, precursor, prodrug, mutein, co-drug thereof and/or pharmaceutically acceptable salt of any of these.

More preferably, the cytokine-like acting substance(s) is a cytokine(s) and/or any peptide, protein, protein-analogue, peptide, protein-variant, derivative, fragment, biopharmaceutical, inductor, precursor, prodrug, mutein, co-drug thereof and/or pharmaceutically acceptable salt of any of these.

The expression “cytokine” or “cytokine(s)” as mentioned in any of the embodiments described herein preferably has to be interpreted broad. Typically, the cytokine(s) activates or is are capable of activating a respective cytokine-receptor(s) of a cell. The cytokine-receptor(s) may for instance be the receptor of the LHs, peripheral T_{reg}s and/or PBMCs. Preferably, the activating or capability of activating is within the skin of the subject, more preferably locally in effective amounts within the skin of the subject, even more preferably not in amounts systemically effective within the subject's body. Preferably the cytokine may be any type of cytokine known to the person skilled in the art. Furthermore, the present invention encompass the use or medical use of precursors, prodrugs and propeptides of such cytokine(s) which are usually not active as such, i.e. have no immunomodulatory activity, but which, upon use in accordance with the present invention, are converted into the actually active cytokine. Hence, these may be used in the method or medical use as described herein or in preparation of the pharmaceutical compositions, injectable dosage form, topical dosage forms, medical devices and/or kit of parts as described herein.

The cytokine(s) may be any peptide, protein, protein-analogue, protein-variant or any derivative, fragment, biopharmaceutical, inductor, precursor, prodrug, mutein, co-drug and/or pharmaceutically acceptable salt of any of these.

The cytokine(s) does not necessarily be derived from or be identical to the cytokine(s) of the same genus and/or species the subject belongs to, it may be any naturally occurring or an artificial cytokine(s), as long as it has a sufficient biologic activity, i.e. an effective cross-reactivity in the subject, particularly is capable of activating the respective cytokine-receptor(s). Preferably, the biologic activity of the cytokine(s) and/or cytokine-like acting substance(s) is sufficient to achieve the treating and/or preventing of the inflammatory disease, immunological disease and/or autoimmunological disease, preferably in the subject.

A cytokine and/or a cytokine-like acting substance typically affects cells by binding and activating the respective cytokine(s)-receptor on the cells. Hence, the activating of the respective receptor typically leads to the affectation of the cell as described above for the immunomodulatory substance. Hence, any embodiment mentioned herein for the immunomodulatory substance relating to the affecting cells like LHs, peripheral T_{reg}s and/or PBMCs is independently and mutatis mutandis applicable to the cytokine(s) and the cytokine-like acting substance.

The activating or the capability of activating the respective cytokine-receptor(s) by the cytokine(s) and/or cytokine-like acting substance(s) may be directly and/or it may be indirectly.

The expression “directly” as mentioned in any of the embodiments described herein typically means in the context of ‘activating’ and/or ‘capable of activating’ the respective receptor that the cytokine(s) or the cytokine-like acting substance(s) acts or is capable of acting by itself as the activator of the respective receptor of the cytokine(s) or the cytokine-like acting substance(s) of a cell like.

The expression “indirectly” as mentioned in any of the embodiments described herein typically means in the context of ‘activating’ and/or ‘capable of activating’ the respective receptor that the cytokine(s) or cytokine-like acting substance(s) is any substance(s) that results in or causes or is capable of resulting in or causing the generation of the cytokine(s) or cytokine-like acting substance(s), such as in particular a precursor, propeptide or prodrug. In other words, the cytokine(s) or cytokine-like acting substance(s) may encompass substances which are usually not active as such, i.e. have no immunomodulatory activity like the activation or capability of activation of a receptor, but which, upon use in accordance with the present invention, are converted into the actually cytokine(s) or cytokine-like acting substance(s). Thereby the desired activation of the respective cytokine(s)-receptor is indirectly effected. This may for instance be the case if the cytokine(s) or the cytokine-like acting substance(s) is any substance(s) like:
an inductor inducing cells to produce and/or secrete the cytokine(s) or the cytokine-like acting substance(s), wherein such cells may be any cells capable of producing and/or secreting the cytokine(s) or the cytokine-like acting substance(s);
a precursor of the cytokine(s) or the cytokine-like acting substance(s) which e.g. is metabolized by the subject's body to generate the cytokine(s) or the cytokine-like acting substance(s). The precursor of the immunomodulatory substance(s) includes, beside others, for instance peptide(s), protein(s), protein-analogue(s), protein-variant(s), propeptide(s), derivative(s) thereof, RNA, DNA, salts, capped immunomodulatory substance(s) etc.;
an inhibitor for a substance, enzyme or factor preventing the expression, interaction, production, secretion and/or activity of the cytokine(s) or the cytokine-like acting substance(s), like for instance particles or nanoparticles or a cap-structure. The cytokine(s) or the cytokine-like acting substance(s) may for instance be packed in particles for delivery. These particles may sterically prevent and protect the substance from interacting with its target, e.g. the receptor, and the substance may for instance exert its action only after the release from the particles in the patient's body;
a prodrug of the cytokine(s) or the cytokine-like acting substance(s); and/or
a co-drug of the cytokine(s) or the cytokine-like acting substance(s).

Preferably, the cytokine(s) and/or cytokine-like acting substance(s) for use according to the present invention and/or the cytokine(s) and/or cytokine-like acting substance(s) of steps (B), (B₁) and/or (C) of the method, the pharmaceutical compositions, injectable dosage forms, topical dosage forms, medical devices and/or kits of parts, is capable of affecting and/or affects the LHs, peripheral T_{reg}s and/or PBMCs. Preferably, the PBMCs are naïve PBMCs, more preferably lymphocytes, even more preferably T-cells and/or B-cells, still more preferably naïve lymphocytes, still even more preferably naïve B-cells and/or naïve T-cells, further preferably naïve T-cells.

More preferably, the cytokine(s) and/or cytokine-like acting substance(s) is capable of directly and/or indirectly affecting and/or affects the LHs, peripheral T_{reg}s and/or PBMCs.

More preferably, the cytokine(s) and/or cytokine-like acting substance(s) is capable of directly and/or indirectly affecting and/or affects the LHs, peripheral T_{reg}s and/or PBMCs in the subject as defined in any of the embodiments according to the present invention, further preferably within the skin of the subject.

If not mentioned otherwise, the description and definition of the immunomodulatory substance(s) stated above is independently and mutatis mutandis applicable to the cytokine(s) and/or cytokine-like acting substance(s). Particular, if not mentioned otherwise, the description and definition of the immunomodulatory substance(s) in respect of “directly” and “indirectly” affecting or capable of affecting the PBMCs is independently and mutatis mutandis applicable to the cytokine(s) and/or cytokine-like acting substance(s).

The cytokine(s) and/or cytokine-like acting substance(s) for use according to the present invention and/or the cytokine(s) and/or cytokine-like acting substance(s) of steps (B), (B₁) and/or (C) of the method, the pharmaceutical compositions, injectable dosage forms, topical dosage forms, medical devices and/or kits of parts, may be similar or identical, preferably identical, to any cytokine(s) and/or cytokine-like acting substance(s) naturally occurring in a vertebrate, preferably a human or a mammal, more preferably in the subject as defined in any of the embodiments according to the present invention. Preferably, the cytokine(s) and/or cytokine-like acting substance(s) may be similar or identical, preferably identical, to any peptide, protein or any fragment of these of a cytokine(s) and/or cytokine-like acting substance(s) naturally occurring in a vertebrate, preferably a human or a mammal as defined above, more preferably in the subject as defined in any of the embodiments according to the present invention.

The cytokine(s) and/or cytokine-like acting substance(s) may comprise, preferably consist of an amino acid sequence which has an amino acid sequence identity to any peptide, protein or any fragment of a cytokine(s) and/or cytokine-like acting substance(s) naturally occurring in a vertebrate, preferably a mammal, more preferably a human or a mammal animal as defined above, more preferably in the subject as defined in any of the embodiments according to the present invention. Preferably, the amino acid sequence identity is 70% or more, even more preferably 85% or more, still more preferably 90% or more, still even more preferably 95% or more, further preferably 97% or more, even further preferably 98% or more, still further preferably 99% or more and 100% or less. Preferably, the amino acid sequence identity includes substitutions, insertions and/or deletions of amino acid residues in respect to the naturally occurring peptide, protein or any fragment of these. Preferably, the total number of substitutions, insertions and/or deletions or the total number combined of substitutions, insertions and/or deletions of amino acid residues is 60 or less, more preferably 40 or less, even more preferably 20 or less, still more preferably 12 or less, still even more preferably 8 or less, further preferably 4 or less, even further preferably 2 or less and zero or more, still further preferably zero.

The cytokine(s) and/or cytokine-like acting substance(s) may be identical to an endogenous immunomodulatory substance(s) of the same genus and/or species the subject belongs to or the amino acid sequence identity refers to an endogenous immunomodulatory substance(s) of the same genus and/or species the subject belongs to. The expression “is identical” typically means in this particular context that the cytokine(s) and/or cytokine-like acting substance(s) is in respect to its primary, secondary, tertiary and quaternary protein structure identical to an/the cytokine(s) and/or cytokine-like acting substance(s) of the same genus and/or species the subject belongs to, more preferably, also in view of the modification pattern, particularly glycosylations.

The cytokine(s) and/or cytokine-like acting substance(s) for use according to the present invention and/or the cytokine(s) and/or cytokine-like acting substance(s) of steps (B), (B₁) and/or (C) of the method, the pharmaceutical compositions, injectable dosage forms, topical dosage forms, medical devices and/or kits of parts, may be derived from an individual belonging to the same genus and/or species as the subject, or which is identical to an/the cytokine(s) and/or cytokine-like acting substance(s) of the same genus and/or species the subject belongs to. For instance, in case the subject is: a human the cytokine(s) and/or cytokine-like acting substance(s) is human cytokine(s) and/or cytokine-like acting substance(s); a cow the cytokine(s) and/or cytokine-like acting substance(s) is bovine cytokine(s) and/or cytokine-like acting substance(s); a horse the cytokine(s) and/or cytokine-like acting substance(s) is equine cytokine(s) and/or cytokine-like acting substance(s); a donkey the cytokine(s) and/or cytokine-like acting substance(s) is donkey cytokine(s) and/or cytokine-like acting substance(s); an elephant the cytokine(s) and/or cytokine-like acting substance(s) is elephant cytokine(s) and/or cytokine-like acting substance(s); a sheep the cytokine(s) and/or cytokine-like acting substance(s) is sheep cytokine(s) and/or cytokine-like acting substance(s); a goat the cytokine(s) and/or cytokine-like acting substance(s) is goat cytokine(s) and/or cytokine-like acting substance(s); a pig the cytokine(s) and/or cytokine-like acting substance(s) is porcine cytokine(s) and/or cytokine-like acting substance(s); a rabbit the cytokine(s) and/or cytokine-like acting substance(s) is rabbit cytokine(s) and/or cytokine-like acting substance(s); a mouse the cytokine(s) and/or cytokine-like acting substance(s) is mouse cytokine(s) and/or cytokine-like acting substance(s); a rat the cytokine(s) and/or cytokine-like acting substance(s) is rat cytokine(s) and/or cytokine-like acting substance(s); a camel the cytokine(s) and/or cytokine-like acting substance(s) is camel cytokine(s) and/or cytokine-like acting substance(s); a dromedary the cytokine(s) and/or cytokine-like acting substance(s) is dromedary cytokine(s) and/or cytokine-like acting substance(s); a lama the cytokine(s) and/or cytokine-like acting substance(s) is lama cytokine(s) and/or cytokine-like acting substance(s); an alpaca the cytokine(s) and/or cytokine-like acting substance(s) is alpaca cytokine(s) and/or cytokine-like acting substance(s); a dog the cytokine(s) and/or cytokine-like acting substance(s) is dog cytokine(s) and/or cytokine-like acting substance(s); and/or a cat the cytokine(s) and/or cytokine-like acting substance(s) is cat cytokine(s) and/or cytokine-like acting substance(s).

Preferably, the cytokine(s) and/or cytokine-like acting substance(s) for use according to the present invention and/or the cytokine(s) and/or cytokine-like acting substance(s) of steps (B), (B₁) and/or (C) of the method, the pharmaceutical compositions, injectable dosage forms, topical dosage forms, medical devices and/or kits of parts, is not the subject's endogenous cytokine(s) and/or cytokine-like acting substance(s). In other words, preferably the cytokine(s) and/or cytokine-like acting substance(s) is not an cytokine(s) and/or cytokine-like acting substance(s) isolated from the subject's body, i.e. from the individual subject to be treated or prevented.

Preferably, the cytokine(s) and/or cytokine-like acting substance(s) for use according to the present invention and/or the cytokine(s) and/or cytokine-like acting substance(s) of steps (B), (B₁) and/or (C) of the method, the pharmaceutical compositions, injectable dosage forms, topical dosage forms, medical devices and/or kits of parts, may be, preferably, is a recombinant cytokine(s) and/or cytokine-like acting substance(s) which may be which may be generated by any recombinant expression techniques known to the person skilled in the art, a chemically synthesized cytokine(s) and/or cytokine-like acting substance(s) synthesized by any production techniques known to the person skilled in the art, an artificially produced cytokine(s) and/or cytokine-like acting substance(s) produced by any production techniques known to the person skilled in the art, a naturally occurring cytokine(s) and/or cytokine-like acting substance(s) obtained from natural sources like an animal or human, or any combination thereof. More preferably, the cytokine(s) and/or cytokine-like acting substance(s) is a recombinant cytokine(s) and/or cytokine-like acting substance(s), chemically synthesized cytokine(s) and/or cytokine-like acting substance(s), artificially produced cytokine(s) and/or cytokine-like acting substance(s) or any combination thereof, even more preferably a recombinant cytokine(s) and/or cytokine-like acting substance(s).

Furthermore, the cytokine(s) and/or cytokine-like acting substance(s) for use according to the present invention and/or the cytokine(s) and/or cytokine-like acting substance(s) of steps (B), (B₁) and/or (C) of the method, the pharmaceutical compositions, injectable dosage forms, topical dosage forms, medical devices and/or kits of parts, may or may not be derivatized, stabilized, fused with other proteins or peptides, conjugated with polymers, contain amino acid analogue(s) or artificial amino acids, modified, covalently or non-covalently, for instance glycosylated or methylated by e.g. posttranslational modification and/or oligomerized, e.g. dimerized or trimerized as long as it is capable of affecting and/or affects LHs, peripheral T_{reg}s and/or PBMCs. More preferably, the modification pattern and/or oligomerization status is similar or identical to the peptide, protein or any fragment of these naturally occurring in a vertebrate, preferably a mammal, more preferably a human or a mammal animal as defined above, more preferably in the subject as defined in any of the embodiments according to the present invention.

Preferably, in any of the embodiments described herein it is intended to administer the cytokine(s) and/or cytokine-like acting substance(s) in an amount low enough to avoid a systemic increase of the cytokine(s) and/or cytokine-like acting substance(s)-concentration in the subject's body. Hence, the cytokine(s) and/or cytokine-like acting substance(s) may be administered in an amount that, when scaled to the entire body of the subject, the resulting systemic concentrations remain in the magnitude of the endogenous levels. Thereby, without wishing to be bound to theory, it is believed that the generation of an increased cytokine(s) and/or cytokine-like acting substance(s)-concentration for instance at sites of inflammation, like inflamed joint. As already stated above, for instance naïve T-cells can mature in the presence of antigen/autoantigen/allergen towards cytotoxic, which may act pro-inflammatory. Thereby decreasing, cancelling or even reversing the beneficial effects achieved by the present invention, particularly, the anti-inflammatory effect of the regulatory T-cells and/or helper T-cells, or of a subset thereof, which are presumably generated.

Preferably, in any of the embodiments described herein, particularly in step (B), (B₁) and/or (C), the cytokine(s) and/or cytokine-like acting substance(s) is administered in an amount that does not cause a systemic increase, more preferably an effective systemic increase, of the concentration of cytokine(s) and/or cytokine-like acting substance(s) in the subject, preferably the blood of the subject, and/or that causes a systemic increase of the concentration of the cytokine(s) and/or cytokine-like acting substance(s). Preferably, cytokine(s) and/or cytokine-like acting substance(s) is administered in an amount low enough that the systemic increase is non-effective.

Preferably, in any of the embodiments described herein, particularly in step (B), (B₁) and/or (C), the cytokine(s) and/or cytokine-like acting substance(s) is administered in an amount that causes a local increase, preferably an effective local increase, of the concentration of the cytokine(s) and/or cytokine-like acting substance(s) in the subject, preferably in the skin tissue of the subject. The skin tissue is preferred, because unlike administration into the blood, no or less flushing away and dilution of the cytokine(s) and/or cytokine-like acting substance(s) takes place.

Preferably, in any of the embodiments described herein, particularly in step (B), (B₁) and/or (C), the generated increased concentration, preferably the generated effective increased concentration of the cytokine(s) and/or cytokine-like acting substance(s) administered is only local but not systemic in the subject.

Preferably, in any of the embodiments described herein, particularly in step (B), (B₁) and/or (C), the cytokine(s) and/or cytokine-like acting substance(s) is administered in an amount that does not cause a systemic activation, preferably an effective systemic activation, of the respective receptor of the cytokine(s) and/or cytokine-like acting substance(s) of a cell, and/or that does not cause a systemic generation, preferably an effective systemic generation, of the cytokine(s) and/or cytokine-like acting substance(s), preferably in the subject.

Preferably, in any of the embodiments described herein, particularly in step (B), (B₁) and/or (C), the cytokine(s) and/or cytokine-like acting substance(s) is administered in an amount that causes a local activation, preferably an effective local activation of the respective receptor of the cytokine(s) and/or cytokine-like acting substance(s) of a cell, and/or causes a local generation, preferably an effective local generation of the cytokine(s) and/or cytokine-like acting substance(s), preferably in the subject. Preferably, the activation and/or generation, more preferably the effective generation and/or effective activation is only local but not systemic in the subject.

Preferably, the cytokine-like acting substance(s) as mentioned in any of the embodiments described herein, specifically the immunomodulatory substance(s) for use according to the present invention and/or the immunomodulatory substance(s) of steps (B), (B₁) and/or (C) of the method, the pharmaceutical compositions, injectable dosage forms, topical dosage forms, medical devices and/or kits of parts, is an cytokine-like acting substance(s), more preferably an interferon-like acting substance(s), interleukin-like acting substance(s) and/or neurotrophin-like acting substance(s).

Preferably, the cytokine(s) as mentioned in any of the embodiments described herein, specifically the immunomodulatory substance(s) for use according to the present invention and/or the immunomodulatory substance(s) of steps (B), (B₁) and/or (C) of the method, the pharmaceutical compositions, injectable dosage forms, topical dosage forms, medical devices and/or kits of parts, is an cytokine(s), more preferably an interferons, interleukins, neurotrophins, colony-stimulating factors, tumour necrosis factors and/or chemokines, even more preferably an interferon(s), interleukin(s) and/or neurotrophin(s), still more preferably an interferon(s) and/or interleukin(s).

The expression “interferon-like acting substance” or “interferon-like acting substance(s)” as mentioned in any of the embodiments described herein can be, preferably is, any substance that exerts a similar or identical effect to an interferon in the body of the subject when administered thereto. The interferon-like acting substance(s) may encompass precursors, prodrugs and propeptides of such substances which are usually not active as such, i.e. have no immunomodulatory activity, but which, upon use in accordance with the present invention, are converted into the actually active interferon-like acting substance(s). Hence, it may be any substance, molecule, peptide, protein, protein-analogue, protein-variant, derivative, fragment, biopharmaceutical, inductor, precursor, prodrug, mutein, co-drug and/or any derivative, fragment and/or pharmaceutically acceptable salt of any of these. Therefore interferon-like acting substance(s) may:
activates or is/are capable of activating a respective interferon-receptor(s) of a cell. The interferon-receptor(s) may for instance be the receptor of the LHs, peripheral T_{reg}s or PBMCs; and/or
results in or causes or is capable of resulting in or causing the generation of a interferon(s) and/or a interferon-like acting substance(s).

Preferably, the interferon-like acting substance(s) activates or is capable of activating the respective interferon-receptor(s). The activating or the capability of activating the respective interferon-receptor(s) may be directly and/or it may be indirectly.

Preferably, the interferon-like acting substance(s) activates or is/are capable of activating a respective interferon-receptor(s) within the skin of the subject. The interferon-like acting substance(s) may be any naturally occurring or artificial interferon-like acting substance(s) as long as it has a sufficient biologic activity, i.e. an effective cross-reactivity in the subject, particularly is capable of activating the respective interferon-receptor(s). Preferably, the interferon-like acting substance(s) is known to the person skilled in the art at the effective date of filing of the present invention.

Preferably, the interferon-like acting substance is any peptide, protein, protein-analogue, protein-variant or any derivative, fragment, biopharmaceutical, inductor, precursor, prodrug, mutein, co-drug and/or pharmaceutically acceptable salt of any of these.

In a preferred embodiment, the interferon-like acting substance(s) may be as detailed below amongst others an interferon(s) and/or any derivative, fragment, protein-analogue, peptide, protein-variant, biopharmaceutical, inductor, precursor, prodrug, mutein, co-drug, propeptide thereof and/or any pharmaceutically acceptable salt of any of these.

More preferably, the interferon-like acting substance(s) is a interferon(s) and/or any derivative, fragment, protein-analogue, peptide, protein-variant, biopharmaceutical, inductor, precursor, prodrug, mutein, co-drug, propeptide thereof and/or any pharmaceutically acceptable salt of any of these.

The expression “interferon” or “interferon(s)” as mentioned in any of the embodiments described herein preferably has to be interpreted broad. Typically, the interferon(s) activates or is are capable of activating a respective interferon-receptor(s) of a cell. The interferon-receptor(s) may for instance be the receptor of the LHs, peripheral T_{reg}s or PBMCs. Preferably, the activating or capability of activating is within the skin of the subject, more preferably locally in effective amounts within the skin of the subject, even more preferably not in amounts systemically effective within the subject's body. Preferably the interferon may be any type of interferon known to the person skilled in the art. Furthermore, the present invention encompass the use or medical use of precursors, prodrugs and propeptides of such interferon(s) which are usually not active as such, i.e. have no immunomodulatory activity, but which, upon use in accordance with the present invention, are converted into the actually active interferon. Hence, these may be used in the method or the medical use as described herein or in preparation of the pharmaceutical compositions, injectable dosage form, topical dosage forms, medical devices and/or kit of parts as described herein.

The interferon(s) may be any peptide, protein, protein-analogue, protein-variant or any derivative, fragment, biopharmaceutical, inductor, precursor, prodrug, mutein, co-drug and/or pharmaceutically acceptable salt of any of these.

The interferon(s) does not necessarily be derived from or be identical to the interferon(s) of the same genus and/or species the subject belongs to, it may be any naturally occurring or an artificial interferon(s), as long as it has a sufficient biologic activity, i.e. an effective cross-reactivity in the subject, particularly is capable of activating the respective interferon-receptor(s). Preferably, the biologic activity of the interferon(s) and/or interferon-like acting substance(s) is sufficient to achieve the treating and/or preventing of the inflammatory disease, immunological disease and/or autoimmunological disease, preferably in the subject.

An interferon and/or an interferon-like acting substance typically affects cells by binding and activating the respective interferon(s)-receptor on the cells. Hence, the activating of the respective receptor typically leads to the affectation of the cell as described above for the immunomodulatory substance. Hence, any embodiment mentioned herein for the immunomodulatory substance relating to the affecting cells like LHs, peripheral T_{reg}s or PBMCs is independently and mutatis mutandis applicable to the interferon(s) and the interferon-like acting substance.

The activating or the capability of activating the respective interferon-receptor(s) by the interferon(s) and/or interferon-like acting substance(s) may be directly and/or it may be indirectly.

If not mentioned otherwise, the description and definition of the cytokine(s) or the cytokine-like acting substance stated above is independently and mutatis mutandis applicable to the interferon(s) and/or interferon-like acting substance(s). Particularly, if not mentioned otherwise, the description and definition of the cytokine(s) or the cytokine-like acting substance in respect of “directly” and “indirectly” activating or capable of activating the respective interferon-receptor(s), is independently and mutatis mutandis applicable to the interferon(s) and/or interferon-like acting substance(s).

Preferably, the interferon(s) and/or interferon-like acting substance(s) for use according to the present invention and/or the interferon(s) and/or interferon-like acting substance(s) of steps (B), (B₁) and/or (C) of the method, the pharmaceutical compositions, injectable dosage forms, topical dosage forms, medical devices and/or kits of parts, is capable of affecting and/or affects the LHs, peripheral T_{reg}s or PBMCs. Preferably, the PBMCs are naïve PBMCs, more preferably lymphocytes, even more preferably T-cells and/or B-cells, still more preferably naïve lymphocytes, still even more preferably naïve B-cells and/or naïve T-cells, further preferably naïve T-cells.

More preferably, the interferon(s) and/or interferon-like acting substance(s) is capable of directly and/or indirectly affecting and/or affects the LHs, peripheral T_{reg}s or PBMCs.

More preferably, the interferon(s) and/or interferon-like acting substance(s) is capable of directly and/or indirectly affecting and/or affects the LHs, peripheral T_{reg}s or PBMCs in the subject as defined in any of the embodiments according to the present invention, further preferably within the skin of the subject.

If not mentioned otherwise, the description and definition of the immunomodulatory substance(s) stated above is independently and mutatis mutandis applicable to the interferon(s) and/or interferon-like acting substance(s). Particularly, if not mentioned otherwise, the description and definition of the immunomodulatory substance(s) in respect of “directly” and “indirectly” affecting or capable of affecting the PBMCs is independently and mutatis mutandis applicable to the interferon(s) and/or interferon-like acting substance(s).

The interferon(s) and/or interferon-like acting substance(s) for use according to the present invention and/or the interferon(s) and/or interferon-like acting substance(s) of steps (B), (B₁) and/or (C) of the method, the pharmaceutical compositions, injectable dosage forms, topical dosage forms, medical devices and/or kits of parts, may be similar or identical, preferably identical, to any interferon(s) and/or interferon-like acting substance(s) naturally occurring in a vertebrate, preferably a human or a mammal, more preferably in the subject as defined in any of the embodiments according to the present invention. Preferably, the interferon(s) and/or interferon-like acting substance(s) may be similar or identical, preferably identical, to any peptide, protein or any fragment of these of a interferon(s) and/or interferon-like acting substance(s) naturally occurring in a vertebrate, preferably a human or a mammal as defined above, more preferably in the subject as defined in any of the embodiments according to the present invention.

The interferon(s) and/or interferon-like acting substance(s) may comprise, preferably consist of an amino acid sequence which has an amino acid sequence identity to any peptide, protein or any fragment of an interferon(s) and/or interferon-like acting substance(s) naturally occurring in a vertebrate, preferably a mammal, more preferably a human or a mammal animal as defined above, more preferably in the subject as defined in any of the embodiments according to the present invention. Preferably, the amino acid sequence identity is 70% or more, even more preferably 85% or more, still more preferably 90% or more, still even more preferably 95% or more, further preferably 97% or more, even further preferably 98% or more, still further preferably 99% or more and 100% or less. Preferably, the amino acid sequence identity includes substitutions, insertions and/or deletions of amino acid residues in respect to the naturally occurring peptide, protein or any fragment of these. Preferably, the total number of substitutions, insertions and/or deletions or the total number combined of substitutions, insertions and/or deletions of amino acid residues is 60 or less, more preferably 40 or less, even more preferably 20 or less, still more preferably 12 or less, still even more preferably 8 or less, further preferably 4 or less, even further preferably 2 or less and zero or more, still further preferably zero.

The interferon(s) and/or interferon-like acting substance(s) may be identical to an endogenous immunomodulatory substance(s) of the same genus and/or species the subject belongs to or the amino acid sequence identity refers to an endogenous immunomodulatory substance(s) of the same genus and/or species the subject belongs to. The expression “is identical” typically means in this particular context that the interferon(s) and/or interferon-like acting substance(s) is in respect to its primary, secondary, tertiary and quaternary protein structure identical to an/the interferon(s) and/or interferon-like acting substance(s) of the same genus and/or species the subject belongs to, more preferably, also in view of the modification pattern, particularly glycosylations.

The interferon(s) and/or interferon-like acting substance(s) for use according to the present invention and/or the interferon(s) and/or interferon-like acting substance(s) of steps (B), (B₁) and/or (C) of the method, the pharmaceutical compositions, injectable dosage forms, topical dosage forms, medical devices and/or kits of parts, may be derived from an individual belonging to the same genus and/or species as the subject, or which is identical to an/the interferon(s) and/or interferon-like acting substance(s) of the same genus and/or species the subject belongs to. For instance, in case the subject is: a human the interferon(s) and/or interferon-like acting substance(s) is human interferon(s) and/or interferon-like acting substance(s); a cow the interferon(s) and/or interferon-like acting substance(s) is bovine interferon(s) and/or interferon-like acting substance(s); a horse the interferon(s) and/or interferon-like acting substance(s) is equine interferon(s) and/or interferon-like acting substance(s); a donkey the interferon(s) and/or interferon-like acting substance(s) is donkey interferon(s) and/or interferon-like acting substance(s); an elephant the interferon(s) and/or interferon-like acting substance(s) is elephant interferon(s) and/or interferon-like acting substance(s); a sheep the interferon(s) and/or interferon-like acting substance(s) is sheep interferon(s) and/or interferon-like acting substance(s); a goat the interferon(s) and/or interferon-like acting substance(s) is goat interferon(s) and/or interferon-like acting substance(s); a pig the interferon(s) and/or interferon-like acting substance(s) is porcine interferon(s) and/or interferon-like acting substance(s); a rabbit the interferon(s) and/or interferon-like acting substance(s) is rabbit interferon(s) and/or interferon-like acting substance(s); a mouse the interferon(s) and/or interferon-like acting substance(s) is mouse interferon(s) and/or interferon-like acting substance(s); a rat the interferon(s) and/or interferon-like acting substance(s) is rat interferon(s) and/or interferon-like acting substance(s); a camel the interferon(s) and/or interferon-like acting substance(s) is camel interferon(s) and/or interferon-like acting substance(s); a dromedary the interferon(s) and/or interferon-like acting substance(s) is dromedary interferon(s) and/or interferon-like acting substance(s); a lama the interferon(s) and/or interferon-like acting substance(s) is lama interferon(s) and/or interferon-like acting substance(s); an alpaca the interferon(s) and/or interferon-like acting substance(s) is alpaca interferon(s) and/or interferon-like acting substance(s); a dog the interferon(s) and/or interferon-like acting substance(s) is dog interferon(s) and/or interferon-like acting substance(s); and/or a cat the interferon(s) and/or interferon-like acting substance(s) is cat interferon(s) and/or interferon-like acting substance(s).

Preferably, the interferon(s) and/or interferon-like acting substance(s) for use according to the present invention and/or the interferon(s) and/or interferon-like acting substance(s) of steps (B), (B₁) and/or (C) of the method, the pharmaceutical compositions, injectable dosage forms, topical dosage forms, medical devices and/or kits of parts, is not the subject's endogenous interferon(s) and/or interferon-like acting substance(s). In other words, preferably the interferon(s) and/or interferon-like acting substance(s) is not an interferon(s) and/or interferon-like acting substance(s) isolated from the subject's body, i.e. from the individual subject to be treated or prevented.

Preferably, the interferon(s) and/or interferon-like acting substance(s) for use according to the present invention and/or the interferon(s) and/or interferon-like acting substance(s) of steps (B), (B₁) and/or (C) of the method, the pharmaceutical compositions, injectable dosage forms, topical dosage forms, medical devices and/or kits of parts, may be, preferably, is a recombinant interferon(s) and/or interferon-like acting substance(s) which may be generated by any recombinant expression techniques known to the person skilled in the art, a chemically synthesized interferon(s) and/or interferon-like acting substance(s) synthesized by any production techniques known to the person skilled in the art, an artificially produced interferon(s) and/or interferon-like acting substance(s) produced by any production techniques known to the person skilled in the art, a naturally occurring interferon(s) and/or interferon-like acting substance(s) obtained from natural sources like an animal or human, or any combination thereof. More preferably, the interferon(s) and/or interferon-like acting substance(s) is a recombinant interferon(s) and/or interferon-like acting substance(s), chemically synthesized interferon(s) and/or interferon-like acting substance(s), artificially produced interferon(s) and/or interferon-like acting substance(s) or any combination thereof, even more preferably a recombinant interferon(s) and/or interferon-like acting substance(s).

Furthermore, the interferon(s) and/or interferon-like acting substance(s) for use according to the present invention and/or the interferon(s) and/or interferon-like acting substance(s) of steps (B), (B₁) and/or (C) of the method, the pharmaceutical compositions, injectable dosage forms, topical dosage forms, medical devices and/or kits of parts, may or may not be derivatized, stabilized, fused with other proteins or peptides, conjugated with polymers, contain amino acid analogue(s) or artificial amino acids, modified, covalently or non-covalently, for instance glycosylated or methylated by e.g. posttranslational modification and/or oligomerized, e.g. dimerized or trimerized as long as it is capable of affecting and/or affects LHs, peripheral T_{reg}s or PBMCs. More preferably, the modification pattern and/or oligomerization status is similar or identical to the peptide, protein or any fragment of these naturally occurring in a vertebrate, preferably a mammal, more preferably a human or a mammal animal as defined above, more preferably in the subject as defined in any of the embodiments according to the present invention.

Preferably, in any of the embodiments described herein it is intended to administer the interferon(s) and/or interferon-like acting substance(s) in an amount low enough to avoid a systemic increase of the interferon(s) and/or interferon-like acting substance(s)-concentration in the subject's body. Hence, the interferon(s) and/or interferon-like acting substance(s) may be administered in an amount that, when scaled to the entire body of the subject, the resulting systemic concentrations remain in the magnitude of the endogenous levels. Thereby, without wishing to be bound to theory, it is believed that the generation of increased interferon(s) and/or interferon-like acting substance(s)-concentration for instance at sites of inflammation, like inflamed joint. As already stated above, for instance naïve T-cells can mature in the presence of antigen/autoantigen/allergen towards cytotoxic, which may act pro-inflammatory. Thereby decreasing, cancelling or even reversing the beneficial effects achieved by the present invention, particularly, the anti-inflammatory effect of the regulatory T-cells and/or helper T-cells, or of a subset thereof, which are presumably generated.

Preferably, in any of the embodiments described herein, particularly in step (B), (B₁) and/or (C), the interferon(s) and/or interferon-like acting substance(s) is administered in an amount that does not cause a systemic increase, more preferably an effective systemic increase, of the concentration of interferon(s) and/or interferon-like acting substance(s) in the subject, preferably the blood of the subject, and/or that causes a systemic increase of the concentration of the interferon(s) and/or interferon-like acting substance(s). Preferably, interferon(s) and/or interferon-like acting substance(s) is administered in an amount low enough that the systemic increase is non-effective.

Preferably, in any of the embodiments described herein, particularly in step (B), (B₁) and/or (C), the interferon(s) and/or interferon-like acting substance(s) is administered in an amount that causes a local increase, preferably an effective local increase, of the concentration of the interferon(s) and/or interferon-like acting substance(s) in the subject, preferably in the skin tissue of the subject.

The skin tissue is preferred, because unlike administration into the blood, no or less flushing away and dilution of the interferon(s) and/or interferon-like acting substance(s) takes place.

Preferably, in any of the embodiments described herein, particularly in step (B), (B₁) and/or (C), the generated increased concentration, preferably the generated effective increased concentration of the interferon(s) and/or interferon-like acting substance(s) administered is only local but not systemic in the subject.

Preferably, in any of the embodiments described herein, particularly in step (B), (B₁) and/or (C), the interferon(s) and/or interferon-like acting substance(s) is administered in an amount that does not cause a systemic activation, preferably an effective systemic activation, of the respective receptor of the interferon(s) and/or interferon-like acting substance(s) of a cell, and/or that does not cause a systemic generation, preferably an effective systemic generation, of the interferon(s) and/or interferon-like acting substance(s), preferably in the subject.

Preferably, in any of the embodiments described herein, particularly in step (B), (B₁) and/or (C), the interferon(s) and/or interferon-like acting substance(s) is administered in an amount that causes a local activation, preferably an effective local activation of the respective receptor of the interferon(s) and/or interferon-like acting substance(s) of a cell, and/or causes a local generation, preferably an effective local generation of the interferon(s) and/or interferon-like acting substance(s), preferably in the subject. Preferably, the activation and/or generation, more preferably the effective generation and/or effective activation is only local but not systemic in the subject.

Preferably, the interferon-like acting substance(s) as mentioned in any of the embodiments described herein, specifically the immunomodulatory substance(s) for use according to the present invention and/or the immunomodulatory substance(s) of steps (B), (B₁) and/or (C) of the method, the pharmaceutical compositions, injectable dosage forms, topical dosage forms, medical devices and/or kits of parts, is an interferon and/or any derivative, fragment, protein-analogue, peptide, protein-variant, biopharmaceutical, inductor, precursor, prodrug, mutein, co-drug, propeptide thereof and/or any pharmaceutically acceptable salt of any of these and/or is an IFN-γ-like acting substance(s).

Preferably, the interferon as mentioned in any of the embodiments described herein, specifically the immunomodulatory substance(s) for use according to the present invention and/or the immunomodulatory substance(s) of steps (B), (B₁) and/or (C) of the method, the pharmaceutical compositions, injectable dosage forms, topical dosage forms, medical devices and/or kits of parts, is IFN-γ.

More preferably, the immunomodulatory substance(s) for use according to the present invention and/or the immunomodulatory substance(s) of steps (B), (B₁) and/or (C) of the method, the pharmaceutical compositions, injectable dosage forms, topical dosage forms, medical devices and/or kits of parts, is IFN-γ and/or any derivative, fragment, protein-analogue, peptide, protein-variant, biopharmaceutical, inductor, precursor, prodrug, mutein, co-drug, propeptide thereof and/or any pharmaceutically acceptable salt of any of these, even more preferably IFN-γ.

The expression “interleukin-like acting substance” or “interleukin-like acting substance(s)” as mentioned in any of the embodiments described herein can be, preferably is, any substance that exerts a similar or identical effect to a interleukin in the body of the subject when administered thereto. The interleukin-like acting substance(s) may encompass precursors, prodrugs and propeptides of such substances which are usually not active as such, i.e. have no immunomodulatory activity, but which, upon use in accordance with the present invention, are converted into the actually active interleukin-like acting substance(s). Hence, it may be any substance, molecule, peptide, protein, protein-analogue, protein-variant, derivative, fragment, biopharmaceutical, inductor, precursor, prodrug, mutein, co-drug and/or any derivative, fragment and/or pharmaceutically acceptable salt of any of these. Therefore interleukin-like acting substance(s) may:
activates or is/are capable of activating a respective interleukin-receptor(s) of a cell. The interleukin-receptor(s) may for instance be the receptor of the LHs, peripheral T_{reg}s or PBMCs; and/or
results in or causes or is capable of resulting in or causing the generation of a interleukin(s) and/or a interleukin-like acting substance(s).

Preferably, the interleukin-like acting substance(s) activates or is capable of activating the respective interleukin-receptor(s). The activating or the capability of activating the respective interleukin-receptor(s) may be directly and/or it may be indirectly.

Preferably, the interleukin-like acting substance(s) activates or is/are capable of activating a respective interleukin-receptor(s) within the skin of the subject. The interleukin-like acting substance(s) may be any naturally occurring or artificial interleukin-like acting substance(s) as long as it has a sufficient biologic activity, i.e. an effective cross-reactivity in the subject, particularly is capable of activating the respective interleukin-receptor(s). Preferably, the interleukin-like acting substance(s) is known to the person skilled in the art at the effective date of filing of the present invention.

Preferably, the interleukin-like acting substance is any peptide, protein, protein-analogue, protein-variant or any derivative, fragment, biopharmaceutical, inductor, precursor, prodrug, mutein, co-drug and/or pharmaceutically acceptable salt of any of these.

In a preferred embodiment, the interleukin-like acting substance(s) may be as detailed below amongst others an interleukin(s) and/or any derivative, fragment, protein-analogue, peptide, protein-variant, biopharmaceutical, inductor, precursor, prodrug, mutein, co-drug, propeptide thereof and/or any pharmaceutically acceptable salt of any of these.

More preferably, the interleukin-like acting substance(s) is an interleukin(s) and/or any derivative, fragment, protein-analogue, peptide, protein-variant, biopharmaceutical, inductor, precursor, prodrug, mutein, co-drug, propeptide thereof and/or any pharmaceutically acceptable salt of any of these.

The expression “interleukin” or “interleukin(s)” as mentioned in any of the embodiments described herein preferably has to be interpreted broad. Typically, the interleukin(s) activates or is are capable of activating a respective interleukin-receptor(s) of a cell. The interleukin-receptor(s) may for instance be the receptor of the LHs, peripheral T_{reg}s or PBMCs. Preferably, the activating or capability of activating is within the skin of the subject, more preferably locally in effective amounts within the skin of the subject, even more preferably not in amounts systemically effective within the subject's body. Preferably the interleukin may be any type of interleukin known to the person skilled in the art. Furthermore, the present invention encompass the use or medical use of precursors, prodrugs and propeptides of such interleukin(s) which are usually not active as such, i.e. have no immunomodulatory activity, but which, upon use in accordance with the present invention, are converted into the actually active interleukin. Hence, these may be used in the method or the medical use as described herein or in preparation of the pharmaceutical compositions, injectable dosage form, topical dosage forms, medical devices and/or kit of parts as described herein.

Preferably, the interleukin(s) may be any peptide, protein, protein-analogue, protein-variant or any derivative, fragment, biopharmaceutical, inductor, precursor, prodrug, mutein, co-drug and/or pharmaceutically acceptable salt of any of these.

The interleukin(s) does not necessarily be derived from or be identical to the interleukin(s) of the same genus and/or species the subject belongs to, it may be any naturally occurring or an artificial interleukin(s), as long as it has a sufficient biologic activity, i.e. an effective cross-reactivity in the subject, particularly is capable of activating the respective interleukin-receptor(s). Preferably, the biologic activity of the interleukin(s) and/or interleukin-like acting substance(s) is sufficient to achieve the treating and/or preventing of the inflammatory disease, immunological disease and/or autoimmunological disease, preferably in the subject.

An interleukin and/or an interleukin-like acting substance typically affects cells by binding and activating the respective interleukin(s)-receptor on the cells. Hence, the activating of the respective receptor typically leads to the affectation of the cell as described above for the immunomodulatory substance. Hence, any embodiment mentioned herein for the immunomodulatory substance relating to the affecting cells like LHs, peripheral T_{reg}s or PBMCs is independently and mutatis mutandis applicable to the interleukin(s) and the interleukin-like acting substance.

The activating or the capability of activating the respective interleukin-receptor(s) by the interleukin(s) and/or interleukin-like acting substance(s) may be directly and/or it may be indirectly.

If not mentioned otherwise, the description and definition of the cytokine(s) or the cytokine-like acting substance stated above is independently and mutatis mutandis applicable to the interleukin(s) and/or interleukin-like acting substance(s). Particularly, if not mentioned otherwise, the description and definition of the cytokine(s) or the cytokine-like acting substance in respect of “directly” and “indirectly” activating or capable of activating the respective interleukin-receptor(s), is independently and mutatis mutandis applicable to the interleukin(s) and/or interleukin-like acting substance(s).

Preferably, the interleukin(s) and/or interleukin-like acting substance(s) for use according to the present invention and/or the interleukin(s) and/or interleukin-like acting substance(s) of steps (B), (B₁) and/or (C) of the method, the pharmaceutical compositions, injectable dosage forms, topical dosage forms, medical devices and/or kits of parts, is capable of affecting and/or affects the LHs, peripheral T_{reg}s or PBMCs. Preferably, the PBMCs are naïve PBMCs, more preferably lymphocytes, even more preferably T-cells and/or B-cells, still more preferably naïve lymphocytes, still even more preferably naïve B-cells and/or naïve T-cells, further preferably naïve T-cells.

More preferably, the interleukin(s) and/or interleukin-like acting substance(s) is capable of directly and/or indirectly affecting and/or affects the LHs, peripheral T_{reg}s or PBMCs.

More preferably, the interleukin(s) and/or interleukin-like acting substance(s) is capable of directly and/or indirectly affecting and/or affects the LHs, peripheral T_{reg}s or PBMCs in the subject as defined in any of the embodiments according to the present invention, further preferably within the skin of the subject.

If not mentioned otherwise, the description and definition of the immunomodulatory substance(s) stated above is independently and mutatis mutandis applicable to the interleukin(s) and/or interleukin-like acting substance(s). Particularly, if not mentioned otherwise, the description and definition of the immunomodulatory substance(s) in respect of “directly” and “indirectly” affecting or capable of affecting the PBMCs is independently and mutatis mutandis applicable to the interleukin(s) and/or interleukin-like acting substance(s).

The interleukin(s) and/or interleukin-like acting substance(s) for use according to the present invention and/or the interleukin(s) and/or interleukin-like acting substance(s) of steps (B), (B₁) and/or (C) of the method, the pharmaceutical compositions, injectable dosage forms, topical dosage forms, medical devices and/or kits of parts, may be similar or identical, preferably identical, to any interleukin(s) and/or interleukin-like acting substance(s) naturally occurring in a vertebrate, preferably a human or a mammal, more preferably in the subject as defined in any of the embodiments according to the present invention. Preferably, the interleukin(s) and/or interleukin-like acting substance(s) may be similar or identical, preferably identical, to any peptide, protein or any fragment of these of a interleukin(s) and/or interleukin-like acting substance(s) naturally occurring in a vertebrate, preferably a human or a mammal as defined above, more preferably in the subject as defined in any of the embodiments according to the present invention.

The interleukin(s) and/or interleukin-like acting substance(s) may comprise, preferably consist of an amino acid sequence which has an amino acid sequence identity to any peptide, protein or any fragment of an interleukin(s) and/or interleukin-like acting substance(s) naturally occurring in a vertebrate, preferably a mammal, more preferably a human or a mammal animal as defined above, more preferably in the subject as defined in any of the embodiments according to the present invention. Preferably, the amino acid sequence identity is 70% or more, even more preferably 85% or more, still more preferably 90% or more, still even more preferably 95% or more, further preferably 97% or more, even further preferably 98% or more, still further preferably 99% or more and 100% or less.

Preferably, the amino acid sequence identity includes substitutions, insertions and/or deletions of amino acid residues in respect to the naturally occurring peptide, protein or any fragment of these. Preferably, the total number of substitutions, insertions and/or deletions or the total number combined of substitutions, insertions and/or deletions of amino acid residues is 60 or less, more preferably 40 or less, even more preferably 20 or less, still more preferably 12 or less, still even more preferably 8 or less, further preferably 4 or less, even further preferably 2 or less and zero or more, still further preferably zero.

The interleukin(s) and/or interleukin-like acting substance(s) may be identical to an endogenous immunomodulatory substance(s) of the same genus and/or species the subject belongs to or the amino acid sequence identity refers to an endogenous immunomodulatory substance(s) of the same genus and/or species the subject belongs to. The expression “is identical” typically means in this particular context that the interleukin(s) and/or interleukin-like acting substance(s) is in respect to its primary, secondary, tertiary and quaternary protein structure identical to an/the interleukin(s) and/or interleukin-like acting substance(s) of the same genus and/or species the subject belongs to, more preferably, also in view of the modification pattern, particularly glycosylations.

The interleukin(s) and/or interleukin-like acting substance(s) for use according to the present invention and/or the interleukin(s) and/or interleukin-like acting substance(s) of steps (B), (B₁) and/or (C) of the method, the pharmaceutical compositions, injectable dosage forms, topical dosage forms, medical devices and/or kits of parts, may be derived from an individual belonging to the same genus and/or species as the subject, or which is identical to an/the interleukin(s) and/or interleukin-like acting substance(s) of the same genus and/or species the subject belongs to. For instance, in case the subject is: a human the interleukin(s) and/or interleukin-like acting substance(s) is human interleukin(s) and/or interleukin-like acting substance(s); a cow the interleukin(s) and/or interleukin-like acting substance(s) is bovine interleukin(s) and/or interleukin-like acting substance(s); a horse the interleukin(s) and/or interleukin-like acting substance(s) is equine interleukin(s) and/or interleukin-like acting substance(s); a donkey the interleukin(s) and/or interleukin-like acting substance(s) is donkey interleukin(s) and/or interleukin-like acting substance(s); an elephant the interleukin(s) and/or interleukin-like acting substance(s) is elephant interleukin(s) and/or interleukin-like acting substance(s); a sheep the interleukin(s) and/or interleukin-like acting substance(s) is sheep interleukin(s) and/or interleukin-like acting substance(s); a goat the interleukin(s) and/or interleukin-like acting substance(s) is goat interleukin(s) and/or interleukin-like acting substance(s); a pig the interleukin(s) and/or interleukin-like acting substance(s) is porcine interleukin(s) and/or interleukin-like acting substance(s); a rabbit the interleukin(s) and/or interleukin-like acting substance(s) is rabbit interleukin(s) and/or interleukin-like acting substance(s); a mouse the interleukin(s) and/or interleukin-like acting substance(s) is mouse interleukin(s) and/or interleukin-like acting substance(s); a rat the interleukin(s) and/or interleukin-like acting substance(s) is rat interleukin(s) and/or interleukin-like acting substance(s); a camel the interleukin(s) and/or interleukin-like acting substance(s) is camel interleukin(s) and/or interleukin-like acting substance(s); a dromedary the interleukin(s) and/or interleukin-like acting substance(s) is dromedary interleukin(s) and/or interleukin-like acting substance(s); a lama the interleukin(s) and/or interleukin-like acting substance(s) is lama interleukin(s) and/or interleukin-like acting substance(s); an alpaca the interleukin(s) and/or interleukin-like acting substance(s) is alpaca interleukin(s) and/or interleukin-like acting substance(s); a dog the interleukin(s) and/or interleukin-like acting substance(s) is dog interleukin(s) and/or interleukin-like acting substance(s); and/or a cat the interleukin(s) and/or interleukin-like acting substance(s) is cat interleukin(s) and/or interleukin-like acting substance(s).

Preferably, the interleukin(s) and/or interleukin-like acting substance(s) for use according to the present invention and/or the interleukin(s) and/or interleukin-like acting substance(s) of steps (B), (B₁) and/or (C) of the method, the pharmaceutical compositions, injectable dosage forms, topical dosage forms, medical devices and/or kits of parts, is not the subject's endogenous interleukin(s) and/or interleukin-like acting substance(s). In other words, preferably the interleukin(s) and/or interleukin-like acting substance(s) is not an interleukin(s) and/or interleukin-like acting substance(s) isolated from the subject's body, i.e. from the individual subject to be treated or prevented.

Preferably, the interleukin(s) and/or interleukin-like acting substance(s) for use according to the present invention and/or the interleukin(s) and/or interleukin-like acting substance(s) of steps (B), (B₁) and/or (C) of the method, the pharmaceutical compositions, injectable dosage forms, topical dosage forms, medical devices and/or kits of parts, may be, preferably, is a recombinant interleukin(s) and/or interleukin-like acting substance(s) which may be generated by any recombinant expression techniques known to the person skilled in the art, a chemically synthesized interleukin(s) and/or interleukin-like acting substance(s) synthesized by any production techniques known to the person skilled in the art, an artificially produced interleukin(s) and/or interleukin-like acting substance(s) produced by any production techniques known to the person skilled in the art, a naturally occurring interleukin(s) and/or interleukin-like acting substance(s) obtained from natural sources like an animal or human, or any combination thereof. More preferably, the interleukin(s) and/or interleukin-like acting substance(s) is a recombinant interleukin(s) and/or interleukin-like acting substance(s), chemically synthesized interleukin(s) and/or interleukin-like acting substance(s), artificially produced interleukin(s) and/or interleukin-like acting substance(s) or any combination thereof, even more preferably a recombinant interleukin(s) and/or interleukin-like acting substance(s).

Furthermore, the interleukin(s) and/or interleukin-like acting substance(s) for use according to the present invention and/or the interleukin(s) and/or interleukin-like acting substance(s) of steps (B), (B₁) and/or (C) of the method, the pharmaceutical compositions, injectable dosage forms, topical dosage forms, medical devices and/or kits of parts, may or may not be derivatized, stabilized, fused with other proteins or peptides, conjugated with polymers, contain amino acid analogue(s) or artificial amino acids, modified, covalently or non-covalently, for instance glycosylated or methylated by e.g. posttranslational modification and/or oligomerized, e.g. dimerized or trimerized as long as it is capable of affecting and/or affects LHs, peripheral T_{reg}s or PBMCs. More preferably, the modification pattern and/or oligomerization status is similar or identical to the peptide, protein or any fragment of these naturally occurring in a vertebrate, preferably a mammal, more preferably a human or a mammal animal as defined above, more preferably in the subject as defined in any of the embodiments according to the present invention.

Preferably, in any of the embodiments described herein it is intended to administer the interleukin(s) and/or interleukin-like acting substance(s) in an amount low enough to avoid a systemic increase of the interleukin(s) and/or interleukin-like acting substance(s)-concentration in the subject's body. Hence, the interleukin(s) and/or interleukin-like acting substance(s) may be administered in an amount that, when scaled to the entire body of the subject, the resulting systemic concentrations remain in the magnitude of the endogenous levels. Thereby, without wishing to be bound to theory, it is believed that the generation of an increased interleukin(s) and/or interleukin-like acting substance(s)-concentration for instance at sites of inflammation, like inflamed joint. As already stated above, for instance naïve T-cells can mature in the presence of antigen/autoantigen/allergen towards cytotoxic, which may act pro-inflammatory. Thereby decreasing, cancelling or even reversing the beneficial effects achieved by the present invention, particularly, the anti-inflammatory effect of the regulatory T-cells and/or helper T-cells, or of a subset thereof, which are presumably generated.

Preferably, in any of the embodiments described herein, particularly in step (B), (B₁) and/or (C), the interleukin(s) and/or interleukin-like acting substance(s) is administered in an amount that does not cause a systemic increase, more preferably an effective systemic increase, of the concentration of interleukin(s) and/or interleukin-like acting substance(s) in the subject, preferably the blood of the subject, and/or that causes a systemic increase of the concentration of the interleukin(s) and/or interleukin-like acting substance(s).

Preferably, interleukin(s) and/or interleukin-like acting substance(s) is administered in an amount low enough that the systemic increase is non-effective.

Preferably, in any of the embodiments described herein, particularly in step (B), (B₁) and/or (C), the interleukin(s) and/or interleukin-like acting substance(s) is administered in an amount that causes a local increase, preferably an effective local increase, of the concentration of the interleukin(s) and/or interleukin-like acting substance(s) in the subject, preferably in the skin tissue of the subject. The skin tissue is preferred, because unlike administration into the blood, no or less flushing away and dilution of the interleukin(s) and/or interleukin-like acting substance(s) takes place.

Preferably, in any of the embodiments described herein, particularly in step (B), (B₁) and/or (C), the generated increased concentration, preferably the generated effective increased concentration of the interleukin(s) and/or interleukin-like acting substance(s) administered is only local but not systemic in the subject.

Preferably, in any of the embodiments described herein, particularly in step (B), (B₁) and/or (C), the interleukin(s) and/or interleukin-like acting substance(s) is administered in an amount that does not cause a systemic activation, preferably an effective systemic activation, of the respective receptor of the interleukin(s) and/or interleukin-like acting substance(s) of a cell, and/or that does not cause a systemic generation, preferably an effective systemic generation, of the interleukin(s) and/or interleukin-like acting substance(s), preferably in the subject.

Preferably, in any of the embodiments described herein, particularly in step (B), (B₁) and/or (C), the interleukin(s) and/or interleukin-like acting substance(s) is administered in an amount that causes a local activation, preferably an effective local activation of the respective receptor of the interleukin(s) and/or interleukin-like acting substance(s) of a cell, and/or causes a local generation, preferably an effective local generation of the interleukin(s) and/or interleukin-like acting substance(s), preferably in the subject. Preferably, the activation and/or generation, more preferably the effective generation and/or effective activation is only local but not systemic in the subject.

Preferably, the interleukin-like acting substance(s) as mentioned in any of the embodiments described herein, specifically the immunomodulatory substance(s) for use according to the present invention and/or the immunomodulatory substance(s) of steps (B), (B₁) and/or (C) of the method, the pharmaceutical compositions, injectable dosage forms, topical dosage forms, medical devices and/or kits of parts, is an interleukin and/or any derivative, fragment, protein-analogue, peptide, protein-variant, biopharmaceutical, inductor, precursor, prodrug, mutein, co-drug, propeptide thereof and/or any pharmaceutically acceptable salt of any of these and/or is an IL-2-like acting substance(s) and/or an IL-4-like acting substance(s).

Preferably, the interleukin as mentioned in any of the embodiments described herein, specifically the immunomodulatory substance(s) for use according to the present invention and/or the immunomodulatory substance(s) of steps (B), (B₁) and/or (C) of the method, the pharmaceutical compositions, injectable dosage forms, topical dosage forms, medical devices and/or kits of parts, is IL-2 and/or IL-4.

More preferably, the immunomodulatory substance(s) for use according to the present invention and/or the immunomodulatory substance(s) of steps (B), (B₁) and/or (C) of the method, the pharmaceutical compositions, injectable dosage forms, topical dosage forms, medical devices and/or kits of parts, is IL-2 and/or IL-4 and/or any derivative, fragment, protein-analogue, peptide, protein-variant, biopharmaceutical, inductor, precursor, prodrug, mutein, co-drug, propeptide thereof and/or any pharmaceutically acceptable salt of any of these, even more preferably IL-2 and/or IL-4.

The expression “neurotrophine-like acting substance” or “neurotrophine-like acting substance(s)” as mentioned in any of the embodiments described herein can be, preferably is, any substance that exerts a similar or identical effect to a neurotrophine in the body of the subject when administered thereto. The neurotrophine-like acting substance(s) may encompass precursors, prodrugs and propeptides of such substances which are usually not active as such, i.e. have no immunomodulatory activity, but which, upon use in accordance with the present invention, are converted into the actually active neurotrophine-like acting substance(s). Hence, it may be any substance, molecule, peptide, protein, protein-analogue, protein-variant, derivative, fragment, biopharmaceutical, inductor, precursor, prodrug, mutein, co-drug and/or any derivative, fragment and/or pharmaceutically acceptable salt of any of these. Therefore neurotrophine-like acting substance(s) may:
activates or is/are capable of activating a respective neurotrophine-receptor(s) of a cell. The neurotrophine-receptor(s) may for instance be the receptor of the LHs, peripheral T_{reg}s or PBMCs; and/or
results in or causes or is capable of resulting in or causing the generation of a neurotrophine(s) and/or a neurotrophine-like acting substance(s).

Preferably, the neurotrophine-like acting substance(s) activates or is capable of activating the respective neurotrophine-receptor(s). The activating or the capability of activating the respective neurotrophine-receptor(s) may be directly and/or it may be indirectly.

Preferably, the neurotrophine-like acting substance(s) activates or is/are capable of activating a respective neurotrophine-receptor(s) within the skin of the subject. The neurotrophine-like acting substance(s) may be any naturally occurring or artificial neurotrophine-like acting substance(s) as long as it has a sufficient biologic activity, i.e. an effective cross-reactivity in the subject, particularly is capable of activating the respective neurotrophine-receptor(s). Preferably, the neurotrophine-like acting substance(s) is known to the person skilled in the art at the effective date of filing of the present invention.

Preferably, the neurotrophine-like acting substance is any peptide, protein, protein-analogue, protein-variant or any derivative, fragment, biopharmaceutical, inductor, precursor, prodrug, mutein, co-drug and/or pharmaceutically acceptable salt of any of these.

In a preferred embodiment, the neurotrophine-like acting substance(s) may be as detailed below amongst others a neurotrophine(s) and/or any derivative, fragment, protein-analogue, peptide, protein-variant, biopharmaceutical, inductor, precursor, prodrug, mutein, co-drug, propeptide thereof and/or any pharmaceutically acceptable salt of any of these.

More preferably, the neurotrophine-like acting substance(s) is a neurotrophine(s) and/or any derivative, fragment, protein-analogue, peptide, protein-variant, biopharmaceutical, inductor, precursor, prodrug, mutein, co-drug, propeptide thereof and/or any pharmaceutically acceptable salt of any of these.

The expression “neurotrophine” or “neurotrophine(s)” as mentioned in any of the embodiments described herein preferably has to be interpreted broad. Typically, the neurotrophine(s) activates or is are capable of activating a respective neurotrophine-receptor(s) of a cell. The neurotrophine-receptor(s) may for instance be the receptor of the LHs, peripheral T_{reg}s or PBMCs. Preferably, the activating or capability of activating is within the skin of the subject, more preferably locally in effective amounts within the skin of the subject, even more preferably not in amounts systemically effective within the subject's body. Preferably the neurotrophine may be any type of neurotrophine known to the person skilled in the art. Furthermore, the present invention encompass the use or medical use of precursors, prodrugs and propeptides of such neurotrophine(s) which are usually not active as such, i.e. have no immunomodulatory activity, but which, upon use in accordance with the present invention, are converted into the actually active neurotrophine. Hence, these may be used in the method or the medical use as described herein or in preparation of the pharmaceutical compositions, injectable dosage form, topical dosage forms, medical devices and/or kit of parts as described herein.

The neurotrophine(s) may be any peptide, protein, protein-analogue, protein-variant or any derivative, fragment, biopharmaceutical, inductor, precursor, prodrug, mutein, co-drug and/or pharmaceutically acceptable salt of any of these.

The neurotrophine(s) does not necessarily be derived from or be identical to the neurotrophine(s) of the same genus and/or species the subject belongs to, it may be any naturally occurring or an artificial neurotrophine(s), as long as it has a sufficient biologic activity, i.e. an effective cross-reactivity in the subject, particularly is capable of activating the respective neurotrophine-receptor(s). Preferably, the biologic activity of the neurotrophine(s) and/or neurotrophine-like acting substance(s) is sufficient to achieve the treating and/or preventing of the inflammatory disease, immunological disease and/or autoimmunological disease, preferably in the subject.

A neurotrophine and/or an neurotrophine-like acting substance typically affects cells by binding and activating the respective neurotrophine(s)-receptor on the cells. Hence, the activating of the respective receptor typically leads to the affectation of the cell as described above for the immunomodulatory substance. Hence, any embodiment mentioned herein for the immunomodulatory substance relating to the affecting cells like LHs, peripheral T_{reg}s or PBMCs is independently and mutatis mutandis applicable to the neurotrophine(s) and the neurotrophine-like acting substance.

The activating or the capability of activating the respective neurotrophine-receptor(s) by the neurotrophine(s) and/or neurotrophine-like acting substance(s) may be directly and/or it may be indirectly.

If not mentioned otherwise, the description and definition of the cytokine(s) or the cytokine-like acting substance stated above is independently and mutatis mutandis applicable to the neurotrophine(s) and/or neurotrophine-like acting substance(s). Particularly, if not mentioned otherwise, the description and definition of the cytokine(s) or the cytokine-like acting substance in respect of “directly” and “indirectly” activating or capable of activating the respective neurotrophine-receptor(s), is independently and mutatis mutandis applicable to the neurotrophine(s) and/or neurotrophine-like acting substance(s).

Preferably, the neurotrophine(s) and/or neurotrophine-like acting substance(s) for use according to the present invention and/or the neurotrophine(s) and/or neurotrophine-like acting substance(s) of steps (B), (B₁) and/or (C) of the method, the pharmaceutical compositions, injectable dosage forms, topical dosage forms, medical devices and/or kits of parts, is capable of affecting and/or affects the LHs, peripheral T_{reg}s or PBMCs. Preferably, the PBMCs are naïve PBMCs, more preferably lymphocytes, even more preferably T-cells and/or B-cells, still more preferably naïve lymphocytes, still even more preferably naïve B-cells and/or naïve T-cells, further preferably naïve T-cells.

More preferably, the neurotrophine(s) and/or neurotrophine-like acting substance(s) is capable of directly and/or indirectly affecting and/or affects the LHs, peripheral T_{reg}s or PBMCs.

More preferably, the neurotrophine(s) and/or neurotrophine-like acting substance(s) is capable of directly and/or indirectly affecting and/or affects the LHs, peripheral T_{reg}s or PBMCs in the subject as defined in any of the embodiments according to the present invention, further preferably within the skin of the subject. If not mentioned otherwise, the description and definition of the immunomodulatory substance(s) stated above is independently and mutatis mutandis applicable to the neurotrophine(s) and/or neurotrophine-like acting substance(s). Particularly, if not mentioned otherwise, the description and definition of the immunomodulatory substance(s) in respect of “directly” and “indirectly” affecting or capable of affecting the PBMCs is independently and mutatis mutandis applicable to the neurotrophine(s) and/or neurotrophine-like acting substance(s).

The neurotrophin(s) and/or neurotrophin-like acting substance(s) for use according to the present invention and/or the neurotrophin(s) and/or neurotrophin-like acting substance(s) of steps (B), (B₁) and/or (C) of the method, the pharmaceutical compositions, injectable dosage forms, topical dosage forms, medical devices and/or kits of parts, may be similar or identical, preferably identical, to any neurotrophin(s) and/or neurotrophin-like acting substance(s) naturally occurring in a vertebrate, preferably a human or a mammal, more preferably in the subject as defined in any of the embodiments according to the present invention. Preferably, the neurotrophin(s) and/or neurotrophin-like acting substance(s) may be similar or identical, preferably identical, to any peptide, protein or any fragment of these of a neurotrophin(s) and/or neurotrophin-like acting substance(s) naturally occurring in a vertebrate, preferably a human or a mammal as defined above, more preferably in the subject as defined in any of the embodiments according to the present invention.

The neurotrophin(s) and/or neurotrophin-like acting substance(s) may comprise, preferably consist of an amino acid sequence which has an amino acid sequence identity to any peptide, protein or any fragment of an neurotrophin(s) and/or neurotrophin-like acting substance(s) naturally occurring in a vertebrate, preferably a mammal, more preferably a human or a mammal animal as defined above, more preferably in the subject as defined in any of the embodiments according to the present invention. Preferably, the amino acid sequence identity is 70% or more, even more preferably 85% or more, still more preferably 90% or more, still even more preferably 95% or more, further preferably 97% or more, even further preferably 98% or more, still further preferably 99% or more and 100% or less. Preferably, the amino acid sequence identity includes substitutions, insertions and/or deletions of amino acid residues in respect to the naturally occurring peptide, protein or any fragment of these. Preferably, the total number of substitutions, insertions and/or deletions or the total number combined of substitutions, insertions and/or deletions of amino acid residues is 60 or less, more preferably or less, even more preferably 20 or less, still more preferably 12 or less, still even more preferably 8 or less, further preferably 4 or less, even further preferably 2 or less and zero or more, still further preferably zero.

The neurotrophin(s) and/or neurotrophin-like acting substance(s) may be identical to an endogenous immunomodulatory substance(s) of the same genus and/or species the subject belongs to or the amino acid sequence identity refers to an endogenous immunomodulatory substance(s) of the same genus and/or species the subject belongs to. The expression “is identical” typically means in this particular context that the neurotrophin(s) and/or neurotrophin-like acting substance(s) is in respect to its primary, secondary, tertiary and quaternary protein structure identical to an/the neurotrophin(s) and/or neurotrophin-like acting substance(s) of the same genus and/or species the subject belongs to, more preferably, also in view of the modification pattern, particularly glycosylations.

The neurotrophin(s) and/or neurotrophin-like acting substance(s) for use according to the present invention and/or the neurotrophin(s) and/or neurotrophin-like acting substance(s) of steps (B), (B₁) and/or (C) of the method, the pharmaceutical compositions, injectable dosage forms, topical dosage forms, medical devices and/or kits of parts, may be derived from an individual belonging to the same genus and/or species as the subject, or which is identical to an/the neurotrophin(s) and/or neurotrophin-like acting substance(s) of the same genus and/or species the subject belongs to. For instance, in case the subject is: a human the neurotrophin(s) and/or neurotrophin-like acting substance(s) is human neurotrophin(s) and/or neurotrophin-like acting substance(s); a cow the neurotrophin(s) and/or neurotrophin-like acting substance(s) is bovine neurotrophin(s) and/or neurotrophin-like acting substance(s); a horse the neurotrophin(s) and/or neurotrophin-like acting substance(s) is equine neurotrophin(s) and/or neurotrophin-like acting substance(s); a donkey the neurotrophin(s) and/or neurotrophin-like acting substance(s) is donkey neurotrophin(s) and/or neurotrophin-like acting substance(s); an elephant the neurotrophin(s) and/or neurotrophin-like acting substance(s) is elephant neurotrophin(s) and/or neurotrophin-like acting substance(s); a sheep the neurotrophin(s) and/or neurotrophin-like acting substance(s) is sheep neurotrophin(s) and/or neurotrophin-like acting substance(s); a goat the neurotrophin(s) and/or neurotrophin-like acting substance(s) is goat neurotrophin(s) and/or neurotrophin-like acting substance(s); a pig the neurotrophin(s) and/or neurotrophin-like acting substance(s) is porcine neurotrophin(s) and/or neurotrophin-like acting substance(s); a rabbit the neurotrophin(s) and/or neurotrophin-like acting substance(s) is rabbit neurotrophin(s) and/or neurotrophin-like acting substance(s); a mouse the neurotrophin(s) and/or neurotrophin-like acting substance(s) is mouse neurotrophin(s) and/or neurotrophin-like acting substance(s); a rat the neurotrophin(s) and/or neurotrophin-like acting substance(s) is rat neurotrophin(s) and/or neurotrophin-like acting substance(s); a camel the neurotrophin(s) and/or neurotrophin-like acting substance(s) is camel neurotrophin(s) and/or neurotrophin-like acting substance(s); a dromedary the neurotrophin(s) and/or neurotrophin-like acting substance(s) is dromedary neurotrophin(s) and/or neurotrophin-like acting substance(s); a lama the neurotrophin(s) and/or neurotrophin-like acting substance(s) is lama neurotrophin(s) and/or neurotrophin-like acting substance(s); an alpaca the neurotrophin(s) and/or neurotrophin-like acting substance(s) is alpaca neurotrophin(s) and/or neurotrophin-like acting substance(s); a dog the neurotrophin(s) and/or neurotrophin-like acting substance(s) is dog neurotrophin(s) and/or neurotrophin-like acting substance(s); and/or a cat the neurotrophin(s) and/or neurotrophin-like acting substance(s) is cat neurotrophin(s) and/or neurotrophin-like acting substance(s).

Preferably, the neurotrophin(s) and/or neurotrophin-like acting substance(s) for use according to the present invention and/or the neurotrophin(s) and/or neurotrophin-like acting substance(s) of steps (B), (B₁) and/or (C) of the method, the pharmaceutical compositions, injectable dosage forms, topical dosage forms, medical devices and/or kits of parts, is not the subject's endogenous neurotrophin(s) and/or neurotrophin-like acting substance(s). In other words, preferably the neurotrophin(s) and/or neurotrophin-like acting substance(s) is not an neurotrophin(s) and/or neurotrophin-like acting substance(s) isolated from the subject's body, i.e. from the individual subject to be treated or prevented.

Preferably, the neurotrophin(s) and/or neurotrophin-like acting substance(s) for use according to the present invention and/or the neurotrophin(s) and/or neurotrophin-like acting substance(s) of steps (B), (B₁) and/or (C) of the method, the pharmaceutical compositions, injectable dosage forms, topical dosage forms, medical devices and/or kits of parts, may be, preferably, is a recombinant neurotrophin(s) and/or neurotrophin-like acting substance(s) which may be generated by any recombinant expression techniques known to the person skilled in the art, a chemically synthesized neurotrophin(s) and/or neurotrophin-like acting substance(s) synthesized by any production techniques known to the person skilled in the art, an artificially produced neurotrophin(s) and/or neurotrophin-like acting substance(s) produced by any production techniques known to the person skilled in the art, a naturally occurring neurotrophin(s) and/or neurotrophin-like acting substance(s) obtained from natural sources like an animal or human, or any combination thereof. More preferably, the neurotrophin(s) and/or neurotrophin-like acting substance(s) is a recombinant neurotrophin(s) and/or neurotrophin-like acting substance(s), chemically synthesized neurotrophin(s) and/or neurotrophin-like acting substance(s), artificially produced neurotrophin(s) and/or neurotrophin-like acting substance(s) or any combination thereof, even more preferably a recombinant neurotrophin(s) and/or neurotrophin-like acting substance(s).

Furthermore, the neurotrophin(s) and/or neurotrophin-like acting substance(s) for use according to the present invention and/or the neurotrophin(s) and/or neurotrophin-like acting substance(s) of steps (B), (B₁) and/or (C) of the method, the pharmaceutical compositions, injectable dosage forms, topical dosage forms, medical devices and/or kits of parts, may or may not be derivatized, stabilized, fused with other proteins or peptides, conjugated with polymers, contain amino acid analogue(s) or artificial amino acids, modified, covalently or non-covalently, for instance glycosylated or methylated by e.g. posttranslational modification and/or oligomerized, e.g. dimerized or trimerized as long as it is capable of affecting and/or affects LHs, peripheral T_{reg}s or PBMCs. More preferably, the modification pattern and/or oligomerization status is similar or identical to the peptide, protein or any fragment of these naturally occurring in a vertebrate, preferably a mammal, more preferably a human or a mammal animal as defined above, more preferably in the subject as defined in any of the embodiments according to the present invention.

Preferably, in any of the embodiments described herein it is intended to administer the neurotrophin(s) and/or neurotrophin-like acting substance(s) in an amount low enough to avoid a systemic increase of the neurotrophin(s) and/or neurotrophin-like acting substance(s)-concentration in the subject's body. Hence, the neurotrophin(s) and/or neurotrophin-like acting substance(s) may be administered in an amount that, when scaled to the entire body of the subject, the resulting systemic concentrations remain in the magnitude of the endogenous levels. Thereby, without wishing to be bound to theory, it is believed that the generation of an increased neurotrophin(s) and/or neurotrophin-like acting substance(s)-concentration for instance at sites of inflammation, like inflamed joint. As already stated above, for instance naïve T-cells can mature in the presence of antigen/autoantigen/allergen towards cytotoxic, which may act pro-inflammatory. Thereby decreasing, cancelling or even reversing the beneficial effects achieved by the present invention, particularly, the anti-inflammatory effect of the regulatory T-cells and/or helper T-cells, or of a subset thereof, which are presumably generated.

Preferably, in any of the embodiments described herein, particularly in step (B), (B₁) and/or (C), the neurotrophin(s) and/or neurotrophin-like acting substance(s) is administered in an amount that does not cause a systemic increase, more preferably an effective systemic increase, of the concentration of neurotrophin(s) and/or neurotrophin-like acting substance(s) in the subject, preferably the blood of the subject, and/or that causes a systemic increase of the concentration of the neurotrophin(s) and/or neurotrophin-like acting substance(s). Preferably, neurotrophin(s) and/or neurotrophin-like acting substance(s) is administered in an amount low enough that the systemic increase is non-effective.

Preferably, in any of the embodiments described herein, particularly in step (B), (B₁) and/or (C), the neurotrophin(s) and/or neurotrophin-like acting substance(s) is administered in an amount that causes a local increase, preferably an effective local increase, of the concentration of the neurotrophin(s) and/or neurotrophin-like acting substance(s) in the subject, preferably in the skin tissue of the subject. The skin tissue is preferred, because unlike administration into the blood, no or less flushing away and dilution of the neurotrophin(s) and/or neurotrophin-like acting substance(s) takes place.

Preferably, in any of the embodiments described herein, particularly in step (B), (B₁) and/or (C), the generated increased concentration, preferably the generated effective increased concentration of the neurotrophin(s) and/or neurotrophin-like acting substance(s) administered is only local but not systemic in the subject.

Preferably, in any of the embodiments described herein, particularly in step (B), (B₁) and/or (C), the neurotrophin(s) and/or neurotrophin-like acting substance(s) is administered in an amount that does not cause a systemic activation, preferably an effective systemic activation, of the respective receptor of the neurotrophin(s) and/or neurotrophin-like acting substance(s) of a cell, and/or that does not cause a systemic generation, preferably an effective systemic generation, of the neurotrophin(s) and/or neurotrophin-like acting substance(s), preferably in the subject.

Preferably, in any of the embodiments described herein, particularly in step (B), (B₁) and/or (C), the neurotrophin(s) and/or neurotrophin-like acting substance(s) is administered in an amount that causes a local activation, preferably an effective local activation of the respective receptor of the neurotrophin(s) and/or neurotrophin-like acting substance(s) of a cell, and/or causes a local generation, preferably an effective local generation of the neurotrophin(s) and/or neurotrophin-like acting substance(s), preferably in the subject. Preferably, the activation and/or generation, more preferably the effective generation and/or effective activation is only local but not systemic in the subject.

Preferably, the neurotrophin-like acting substance(s) as mentioned in any of the embodiments described herein, specifically the immunomodulatory substance(s) for use according to the present invention and/or the immunomodulatory substance(s) of steps (B), (B₁) and/or (C) of the method, the pharmaceutical compositions, injectable dosage forms, topical dosage forms, medical devices and/or kits of parts, is a neurotrophin, a BDNF-like acting substance(s) and/or a NGF-like acting substance(s), more preferably a neurotrophin and/or a BDNF-like acting substance(s).

Preferably, the neurotrophin as mentioned in any of the embodiments described herein, specifically the immunomodulatory substance(s) for use according to the present invention and/or the immunomodulatory substance(s) of steps (B), (B₁) and/or (C) of the method, the pharmaceutical compositions, injectable dosage forms, topical dosage forms, medical devices and/or kits of parts, is BDNF and/or NGF (nerve growth factor), more preferably BDNF.

More preferably, the immunomodulatory substance(s) for use according to the present invention and/or the immunomodulatory substance(s) of steps (B), (B₁) and/or (C) of the method, the pharmaceutical compositions, injectable dosage forms, topical dosage forms, medical devices and/or kits of parts, is BDNF, NGF (nerve growth factor) and/or any derivative, fragment, protein-analogue, peptide, protein-variant, biopharmaceutical, inductor, precursor, prodrug, mutein, co-drug, propeptide thereof and/or any pharmaceutically acceptable salt of any of these, even more preferably BDNF.

The “skin-conditioning agent” the as mentioned in any of the embodiments described herein, specifically the skin-conditioning agent for use according to the present invention and/or the skin-conditioning agent administered in step (A) may be, preferably is any type or kind of substance(s), composition or formulation suitable for modifying a condition of the skin particularly in respect of the amount of PBMCs, the vasodilation of the capillaries within the skin, the blood volume within the skin, the sO₂ within the skin, the rHb within the skin, the temperature on the skin and/or the redness on the skin.

More preferably, the skin-conditioning agent preferably is any type or kind of substance(s), composition or formulation suitable to generate and/or generates:
an accumulation of PBMCs within the skin, wherein it is to be understood that preferably any embodiment or definition thereof which is described under the outline note ‘As further regards step (A-0) for any of the embodiments as described herein’ is independently and mutatis mutandis applicable; and/or
a vasodilation of the capillaries within the skin, wherein it is to be understood that preferably any embodiment or definition thereof which is described under the outline note ‘As further regards step (A-1) for any of the embodiments as described herein’ is independently and mutatis mutandis applicable; and/or
an increased blood volume within the skin, wherein it is to be understood that preferably any embodiment or definition thereof which is described under the outline note ‘As further regards step (A-2) for any of the embodiments as described herein’ is independently and mutatis mutandis applicable; and/or
an increased sO₂ (oxygen saturation of haemoglobin) and/or an increased rHb (relative haemoglobin amount) within the skin, wherein it is to be understood that preferably any embodiment or definition thereof which is described under the outline note ‘As further regards step (A-3) for any of the embodiments as described herein’ is independently and mutatis mutandis applicable; and/or
an increased temperature on the skin, wherein it is to be understood that preferably any embodiment or definition thereof which is described under the outline note ‘As further regards step (A-4) for any of the embodiments as described herein’ is independently and mutatis mutandis applicable; and/or
a redness on the skin, wherein it is to be understood that preferably any embodiment or definition thereof which is described under the outline note ‘As further regards step (A-5) for any of the embodiments as described herein’ is independently and mutatis mutandis applicable.

Even more preferably, the skin-conditioning agent is suitable and/or sufficient to generate and/or generates the vasodilation of the capillaries within the skin, the increased blood volume within the skin, the increased sO₂ and/or an increased rHb within the skin and/or the increased temperature on the skin. Still more preferably, the vasodilation of the capillaries within the skin, the increased sO₂ and/or the increased rHb within the skin, and/or the increased temperature on the skin. Still even more preferably, the increased sO₂ and/or the increased rHb within the skin, and/or the increased temperature on the skin.

More preferably, the skin-conditioning agent does not cause an allergic reaction in the subject.

Preferably, the skin-conditioning agent is any blood-circulation-increasing agent, vasodilating agent, skin-temperature increasing agent, skin-sO₂-increasing agent and/or skin-rHb-increasing agent. Examples for the skin-conditioning agent comprise nitrates, alpha blockers, ACE-inhibitors, ginkgo preparations like gingko balm, calcium antagonists, dihydralazine, minoxidil, dihydroergotoxin, nicotinic acid analogues, vasodilators like methylnicotinat, moxa herbs, capsaicine, and/or pentoxifylline, a heat crème, a vasodilator containing crème, a methylnicotinat containing crème, particularly Kytta® heat balm (Kytta® heat balm containing methylnicotinat [“Kytta® heat balm” by P&G Health Germany GmbH, Germany, PZN 12358936]) or any combination thereof. Preferably, the skin-conditioning agent comprises as active ingredient(s), preferably consists of as active ingredient(s) of, nitrates, alpha blockers, ACE-inhibitors, ginkgo preparations like gingko balm, calcium antagonists, dihydralazine, minoxidil, dihydroergotoxin, nicotinic acid analogues, vasodilators like methylnicotinat, moxa herbs, capsaicine, pentoxifylline, vasodilator containing crème, methylnicotinat containing crème, Kytta® heat balm or any combination thereof, more preferably methylnicotinat containing crème and/or methylnicotinat, even more preferably Kytta® heat balm and/or methylnicotinat.

Preferably, the skin-conditioning agent is in the form of any of the pharmaceutical compositions, the injectable dosage forms, the topical dosage forms, medical devices and/or the kits of parts according to the present invention or as mentioned in any of the embodiments described herein.

Preferably, the blood-circulation-increasing agent is an agent suitable for increasing and/or increasing the blood volume within the skin, i.e. a blood-volume increasing agent. Even more preferably, the blood-circulation-increasing agent is a blood-volume increasing agent.

It is to be understood, that the expression:
“[ . . . ] wherein the method comprises, preferably consists of the steps of
   (A) which is selected from one or more of steps (A-0), (A-1), (A-2), (A-3), (A-4), (A-5), (A-6), (A-7) and/or (A-8); and is equivalent in its meaning and can be used interchangeably with the expression:
“[ . . . ] wherein the method comprises a step (A), wherein step (A) is selected from one or more of steps:
   (A-0) generating an accumulation of PBMCs (peripheral blood mononuclear cells) within the skin of the subject;
   (A-1) generating a vasodilation of the capillaries within the skin of the subject;
   (A-2) generating an increased blood volume within the skin of the subject;
   (A-3) generating an increased sO₂ (oxygen saturation of haemoglobin) within the skin and/or an increased rHb (relative haemoglobin amount) within the skin of the subject;
   (A-4) generating an increased temperature on the skin of the subject;
   (A-5) generating a redness on the skin of the subject;
   (A-6) administering conditioning energy to the skin of the subject;
   (A-7) administering a skin-conditioning agent to the skin of the subject; and/or
   (A-8) administering PBMCs into the skin of the subject,
and the method further comprises the step(s) of: [ . . . ]”.

As already stated above, the present invention relates to an immunomodulatory substance(s) and/or a skin-conditioning agent for use in a method for treating and/or preventing an inflammatory disease, immunological disease and/or autoimmunological disease in a subject,
wherein the method comprises, preferably consists of, the steps of:
(A) which is selected from one or more of steps (A-0), (A-1), (A-2), (A-3), (A-4), (A-5), (A-6), (A-7) and/or (A-8); and
(B) administering the immunomodulatory substance(s) to the skin of said subject,
or
wherein the method comprises, preferably consists of, the steps of:
(A) which is selected from one or more of steps (A-0), (A-1), (A-2), (A-3), (A-4), (A-5), (A-6), (A-7) and/or (A-8); and
(C) administering the immunomodulatory substance(s) to the skin of said subject,
or
wherein the method comprises, preferably consists of, the steps of:
(A) which is selected from one or more of steps (A-0), (A-1), (A-2), (A-3), (A-4), (A-5), (A-6), (A-7) and/or (A-8); and
(B) administering the immunomodulatory substance(s) to the skin of said subject; and
(C) administering the immunomodulatory substance(s) to the skin of the subject,
wherein the immunomodulatory substance(s) administered in step (C) is different from the immunomodulatory substance(s) administered in step (B),
and to any of said methods as such.

Preferably, the present invention relates to an immunomodulatory substances and/or a skin-conditioning agent for use in a method for treating and/or preventing an inflammatory disease, immunological disease and/or autoimmunological disease in a subject, wherein step (B) is performed a second time as (B₁), wherein the method comprises, preferably consists of, the steps of:
(A) which is selected from one or more of steps (A-0), (A-1), (A-2), (A-3), (A-4), (A-5), (A-6), (A-7) and/or (A-8); and
(B) administering the immunomodulatory substance(s) to the skin of said subject; and
(B₁) administering the immunomodulatory substance(s) to the skin of said subject,
wherein the immunomodulatory substance(s) administered in step (B₁) is different from the immunomodulatory substance(s) administered in step (B).

More preferably, the present invention relates to an immunomodulatory substances and/or a skin-conditioning agent for use in a method for treating and/or preventing an inflammatory disease, immunological disease and/or autoimmunological disease in a subject, wherein the method comprises, preferably consists of, the steps of:
(A) which is selected from one or more of steps (A-0), (A-1), (A-2), (A-3), (A-4), (A-5), (A-6), (A-7) and/or (A-8); and
(B) administering the immunomodulatory substance(s) to the skin of said subject; and
(B₁) administering the immunomodulatory substance(s) to the skin of said subject; and
(C) administering the immunomodulatory substance(s) to the skin of said subject,
wherein the immunomodulatory substance(s) administered in step (B), step (B₁) and step (C) are different from each other.

Even more preferably, the present invention relates to an immunomodulatory substances and/or a skin-conditioning agent for use in a method for treating and/or preventing an inflammatory disease, immunological disease and/or autoimmunological disease in a subject,
and the method comprises, preferably consists of:
   a first set of steps, comprising, preferably, consisting of:
   (A) which is selected from one or more of steps (A-0), (A-1), (A-2), (A-3), (A-4), (A-5), (A-6), (A-7) and/or (A-8); and
   (B) administering the immunomodulatory substance(s) to the skin of said subject; and
   (C) administering the immunomodulatory substance(s) to the skin of the subject, and
   a second set of steps comprising, preferably consisting of:
   (A) which is selected from one or more of steps (A-0), (A-1), (A-2), (A-3), (A-4), (A-5), (A-6), (A-7) and/or (A-8); and
   (B₁) administering the immunomodulatory substance(s) to the skin of said subject; and
   (C) administering the immunomodulatory substance(s) to the skin of the subject,
wherein the immunomodulatory substance(s) administered in step (B₁) is different from the immunomodulatory substance(s) administered in step (B), and
wherein the immunomodulatory substance(s) administered in each of steps (C) is different from the immunomodulatory substance(s) administered in step (B) and step (B₁).

Preferably, the immunomodulatory substance(s) administered in each of steps (C) are the same or different, more preferably are the same.

If not mentioned otherwise, it is to be understood that any embodiment of step (A) described herein is independently applicable to each of steps (A). Hence, step (A) of the first set of steps may or may not be performed the same as step (A) of the second set of steps, for instance in respect to the way of administering a skin-conditioning agent, the used concentration, whether administered by topical application or by injection, the site of the skin area on the patients bod and/or the size of the skin area, etc. More preferably, steps (A) are all performed by topical application or by injection.

If not mentioned otherwise, it is to be understood that any embodiment of step (C) described herein is independently applicable to each of steps (C). Hence, step (C) of the first set of steps may or may not be performed the same as step (C) of the second set of steps, for instance in respect to the type of immunomodulatory substance(s), the used concentration, whether administered by topical application or by injection, the size of the skin area etc. More preferably, steps (C) are performed the same. Hence, in step (C) of the first set of steps, for instance, the administration of the immunomodulatory substance(s) may be applied by injection while in step (C) of the second set of steps an administration by topical application may be used.

More preferably, the present invention relates to an immunomodulatory substance(s) and/or a skin-conditioning agent for use in a method for treating and/or preventing an inflammatory disease, immunological disease and/or autoimmunological disease in a subject, wherein the method comprises, preferably consists of, the steps of:
(A) which is selected from one or more of steps (A-0), (A-1), (A-2), (A-3), (A-4), (A-5), (A-6), (A-7) and/or (A-8); and
(B) administering the immunomodulatory substance(s) to the skin of said subject,
or
even more preferably, wherein the method comprises, preferably consists of, the steps of:
(A) which is selected from one or more of steps (A-0), (A-1), (A-2), (A-3), (A-4), (A-5), (A-6), (A-7) and/or (A-8); and
(B) administering the immunomodulatory substance(s) to the skin of said subject; and
(C) administering the immunomodulatory substance(s) to the skin of the subject,
wherein the immunomodulatory substance(s) administered in step (C) is different from the immunomodulatory substance(s) administered in step (B),
and to any of said methods as such.

Hence, more preferably the immunomodulatory substance(s) as mentioned in any of the embodiments described herein, specifically the immunomodulatory substance(s) for use according to the present invention and/or the immunomodulatory substance(s) of steps (B), (B₁) and/or (C) of the method, the pharmaceutical compositions, injectable dosage forms, topical dosage forms, medical devices and/or kits of parts, is:
the cytokine-like acting substance(s);
even more preferably the immune-related cytokine-like acting substance(s);
still more preferably the interferon-like acting substance(s), interleukin-like acting substance(s) and/or neurotrophin-like acting substance(s);
still even more preferably the IFN-γ-like acting substance(s), IL-4-like acting substance(s), BDNF-like acting substance(s) and/or IL-2-like acting substance(s);
further preferably the IFN-γ-like acting substance(s), IL-4-like acting substance(s) and/or BDNF-like acting substance(s);
even further the IFN-γ-like acting substance(s), IL-4-like acting substance(s) and/or IL-2-like acting substance(s), or IL-4-like acting substance(s), BDNF-like acting substance(s) and/or IL-2-like acting substance(s);
still further preferably the IFN-γ-like acting substance(s) and/or the IL-4-like acting substance(s); or the IL-4-like acting substance(s) and/or the BDNF-like acting substance(s);
still even further preferably the IFN-γ-like acting substance(s) and/or the IL-2-like acting substance(s); or the IL-4-like acting substance(s) and/or the BDNF-like acting substance(s);
furthermore preferably the IFN-γ-like acting substance(s); or the IL-4-like acting substance(s) and/or BDNF-like acting substance(s); or the IL-2-like acting substance(s).

Even more preferably, the immunomodulatory substance(s) as mentioned in any of the embodiments described herein, specifically the immunomodulatory substance(s) for use according to the present invention and/or the immunomodulatory substance(s) of steps (B), (B₁) and/or (C) of the method, the pharmaceutical compositions, injectable dosage forms, topical dosage forms, medical devices and/or kits of parts, is
the cytokine(s);
still more preferably the immune-related cytokine(s);
still even more preferably the interferon(s), interleukin(s) and/or neurotrophin(s);
further preferably the IFN-γ, IL-4, BDNF and/or IL-2;
even further preferably the IFN-γ, IL-4 and/or BDNF;
still further preferably the IFN-γ, IL-4 and/or IL-2; or the IL-4, BDNF and/or IL-2;
still even further preferably the IFN-γ and/or IL-4; or the IL-4 and/or BDNF;
furthermore preferably the IFN-γ and/or IL-2; or the IL-4 and/or BDNF;
even furthermore preferably the IFN-γ; or the IL-4 and/or BDNF; or the IL-2,
and/or any derivative, fragment, protein-analogue, peptide, protein-variant, biopharmaceutical, inductor, precursor, prodrug, mutein, co-drug, propeptide thereof and/or any pharmaceutically acceptable salt of any of these.

For the sake of simplicity and clarity, the following embodiments were only written out in full length for the cytokine(s), interferon(s), interleukin(s), neurotrophin(s), IFN-γ, IL-4, BDNF and/or IL-2. If not mentioned otherwise, it is to be understood that any of the embodiments of the present invention stated generally herein for the cytokine(s), interferon(s), interleukin(s), neurotrophin(s), IFN-γ, IL-4, BDNF and/or IL-2 and particularly in the following paragraphs in terms of:
the method comprising steps (A) and (C); steps (A), (B) and (C), steps (A) and (B); steps (A) and (B), wherein step (B) is performed a second time as (B₁); or a first set of steps (A), (B) and (C) and a second set of steps (A), (B₁) and (C); and/or
the particular inflammatory disease, immunological disease and/or autoimmunological disease to be treated and/or prevented;
and/or
any combination of any of these,
is independently and mutatis mutandis applicable to the cytokine-like acting substance(s), interferon-like acting substance(s), interleukin-like acting substance(s), neurotrophin-like acting substance(s), IFN-γ-like acting substance(s), IL-2-like acting substance(s), IL-4-like acting substance(s) and/or BDNF-like acting substance(s), wherein for that reason, where applicable, the term ‘cytokine(s)’ is to be understood cytokine-like acting substance(s)′, ‘interferon(s)’ is to be understood ‘interferon-like acting substance(s)’, ‘interleukin(s)’ is to be understood ‘interleukin-like acting substance(s)’, ‘neurotrophin(s)’ is to be understood ‘neurotrophin-like acting substance(s)’, ‘IFN-γ’ is to be understood IFN-γ-like acting substance(s)′, ‘IL-4’ is to be understood ‘IL-4-like acting substance(s)’, ‘BDNF’ is to be understood ‘BDNF-like acting substance(s)’ and ‘IL-2’ is to be understood ‘IL-2-like acting substance(s)’. Preferably, in one particular embodiment all of such terms are replaced or all of such terms remain unchanged, however, not some replaced and some not. The embodiments referring to cytokine(s), interferon(s), neurotrophin(s), interleukin(s), IFN-γ, IL-4, BDNF, IL-2 and/or any derivative, fragment, protein-analogue, peptide, protein-variant, biopharmaceutical, inductor, precursor, prodrug, mutein, co-drug, propeptide thereof and/or any pharmaceutically acceptable salt of any of these are more preferred. The embodiments referring to cytokine(s), interferon(s), neurotrophin(s), interleukin(s), IFN-γ, IL-4, BDNF and/or IL-2 are even more preferred.

Preferably,
the method comprises steps (A) and (C);
the immunomodulatory substance(s) for use according to the present invention is any of the immunomodulatory substance(s) as described herein, that is the immunomodulatory substance(s) including IL-2, preferably a cytokine(s) including IL-2, more preferably an interferon(s), neurotrophin(s) and/or interleukin(s) including IL-2, even more preferably IL-2; and
the immunomodulatory substance(s) in step (C) is IL-2,
or
the method comprises steps (A), (B) and (C);
the immunomodulatory substance(s) for use according to the present invention is any of the immunomodulatory substance(s) as described herein, that is the immunomodulatory substance(s) including IL-2, preferably a cytokine(s) including IL-2, more preferably an interferon(s), neurotrophin(s) and/or interleukin(s) including IL-2, even more preferably IL-2;
the immunomodulatory substance(s) in step (B) is any of the immunomodulatory substance(s) as described herein except for IL-2; and
the immunomodulatory substance(s) in step (C) is IL-2,
or
the method comprises steps (A) and (B);
the immunomodulatory substance(s) for use according to the present invention is IFN-γ, IL-4 and/or BDNF, preferably IFN-γ and/or IL-4; and
the immunomodulatory substance(s) in step (B) is IFN-γ and/or IL-4, or IL-4 and/or BDNF; preferably is IFN-γ, or s IL-4 and/or BDNF; more preferably is IFN-γ or IL-4,
or
the method comprises steps (A), (B) and (C);
the immunomodulatory substance(s) for use according to the present invention is IFN-γ, IL-4, BDNF and/or IL-2;
the immunomodulatory substance(s) in step (B) is IFN-γ and/or IL-4, or IL-4 and/or BDNF; preferably is IFN-γ, or IL-4 and/or BDNF; more preferably is IFN-γ or IL-4; and
the immunomodulatory substance(s) in step (C) is IL-2.

Even more preferably,
the method comprises steps (A), (B) and (C);
   the immunomodulatory substance(s) for use according to the present invention is IFN-γ and/or IL-2;
   the immunomodulatory substance(s) in step (B) is IFN-γ; and
   the immunomodulatory substance(s) in step (C) is IL-2,
   or
   the immunomodulatory substance(s) for use according to the present invention is IL-4 and/or IL-2;
   the immunomodulatory substance(s) in step (B) is IL-4; and
   the immunomodulatory substance(s) in step (C) is IL-2,
or the method comprises steps (A) and (B), wherein step (B) is performed a second time as (B₁);
   the immunomodulatory substance(s) for use according to the present invention is IFN-γ and/or IL-4;
   the immunomodulatory substance(s) in step (B) is IFN-γ; and
   the immunomodulatory substance(s) in step (B₁) is IL-4,
or
   the immunomodulatory substance(s) for use according to the present invention is IL-4 and/or BDNF;
   the immunomodulatory substance(s) in step (B) is IL-4; and
   the immunomodulatory substance(s) in step (B₁) is BDNF.

The embodiment, in which the method comprises steps (A), (B) and (C) is still more preferred.

If not mentioned otherwise, it is to be understood that any embodiment or definition described herein in terms of step (B) is independently and mutatis mutandis applicable to step (B₁) as mentioned in any of the embodiments described herein.

Still even more preferably, the method comprises steps (A), (B), (B₁) and (C);
the immunomodulatory substance(s) for use according to the present invention is IFN-γ, IL-4 and/or IL-2;
the immunomodulatory substance(s) in step (B) is IFN-γ;
the immunomodulatory substance(s) in step (B₁) is IL-4; and
the immunomodulatory substance(s) in step (C) is IL-2,
or
the immunomodulatory substance(s) for use according to the present invention is IL-4, BDNF and/or IL-2;
the immunomodulatory substance(s) in step (B) is IL-4;
the immunomodulatory substance(s) in step (B₁) is BDNF; and the immunomodulatory substance(s) in step (C) is IL-2.

Further preferably,
the immunomodulatory substance(s) for use according to the present invention is IFN-γ, IL-4 and/or IL-2, and the method comprises:
a first set of steps, comprising:
(A) which is selected from one or more of steps (A-0), (A-1), (A-2), (A-3), (A-4), (A-5), (A-6), (A-7) and/or (A-8); and
(B) administering IFN-γ to the skin of said subject; and
(C) administering IL-2 to the skin of the subject, and a second set of steps comprising:
(A) which is selected from one or more of steps (A-0), (A-1), (A-2), (A-3), (A-4), (A-5), (A-6), (A-7) and/or (A-8); and
(B₁) administering IL-4 to the skin of said subject; and
(C) administering IL-2 to the skin of the subject,
or
the immunomodulatory substance(s) for use according to the present invention is IL-4, BDNF and/or IL-2, and the method comprises
a first set of steps, comprising:
(A) which is selected from one or more of steps (A-0), (A-1), (A-2), (A-3), (A-4), (A-5), (A-6), (A-7) and/or (A-8); and
(B) administering IL-4 to the skin of said subject; and
(C) administering IL-2 to the skin of the subject, and
a second set of steps comprising:
(A) which is selected from one or more of steps (A-0), (A-1), (A-2), (A-3), (A-4), (A-5), (A-6), (A-7) and/or (A-8);
and
(B₁) administering BDNF to the skin of said subject; and
(C) administering IL-2 to the skin of the subject.

In any of the embodiments described herein comprising steps (A) and (B), still further preferably,
the immunomodulatory substance(s) for use according to the present invention is an interferon(s), neurotrophin(s) and/or interleukin(s);
the immunomodulatory substance(s) in step (B) is the interferon(s), neurotrophin(s) and/or interleukin(s), still even further preferably,
the immunomodulatory substance(s) for use according to the present invention is IFN-γ, IL-4 and/or BDNF; and
the immunomodulatory substance(s) in step (B) is IFN-γ, IL-4 and/or BDNF, furthermore preferably,
the immunomodulatory substance(s) for use according to the present invention is IFN-γ and/or IL-4 or IL-4 and/or BDNF; and
the immunomodulatory substance(s) in step (B) is IFN-γ and/or IL-4 or is IL-4 and/or BDNF, even furthermore preferably,
the immunomodulatory substance(s) for use according to the present invention is IFN-γ, IL-4 and/or BDNF; and
the immunomodulatory substance(s) in step (B) is IFN-γ and/or IL-4 or is IL-4 and/or BDNF, still furthermore preferably,
the immunomodulatory substance(s) for use according to the present invention is IFN-γ or IL-4; and
the immunomodulatory substance(s) in step (B) is IFN-γ or IL-4.

In any of the embodiments described herein comprising steps (A) and (C), still further preferably,
the immunomodulatory substance(s) for use according to the present invention is a cytokine(s); and
the immunomodulatory substance(s) in step (C) is the cytokine(s), still even further preferably,
the immunomodulatory substance(s) for use according to the present invention is an interleukin(s); and
the immunomodulatory substance(s) in step (C) is the interleukin(s), furthermore preferably,
the immunomodulatory substance(s) for use according to the present invention is IL-2; and
the immunomodulatory substance(s) in step (C) is IL-2.

In any of the embodiments described herein comprising steps (A), (B) and (C), still further preferably,
the immunomodulatory substance(s) for use according to the present invention is any of the immunomodulatory substance(s) as described herein including IL-2 as described herein, more preferably a cytokine(s) including IL-2;
the immunomodulatory substance(s) in step (B) is any of the immunomodulatory substance(s) as described herein except for IL-2, more preferably the cytokine(s) except for IL-2; and
the immunomodulatory substance(s) in step (C) is IL-2,
still even further preferably,
the immunomodulatory substance(s) for use according to the present invention is an interferon(s), neurotrophin(s) and/or interleukin(s) including IL-2;
the immunomodulatory substance(s) in step (B) is the interferon(s), neurotrophin(s) and/or interleukin(s) except for IL-2;
and
the immunomodulatory substance(s) in step (C) is IL-2,
furthermore preferably,
the immunomodulatory substance(s) for use according to the present invention is IFN-γ, IL-4, BDNF and/or IL-2;
the immunomodulatory substance(s) in step (B) is IFN-γ, IL-4 and/or BDNF, more preferably IFN-γ and/or IL-4 or IL-4 and/or BDNF; and
the immunomodulatory substance(s) in step (C) is IL-2,
even furthermore preferably,
the immunomodulatory substance(s) for use according to the present invention is IFN-γ, IL-4 and/or IL-2, preferably IFN-γ and/or IL-2 or IL-4 and/or IL-2;
the immunomodulatory substance(s) in step (B) is IFN-γ and/or IL-4, preferably IFN-γ or IL-4; and
the immunomodulatory substance(s) in step (C) is IL-2.

Preferably, in all embodiments of the present invention described herein relating to IFN-γ, that is all embodiments mentioning IFN-γ alone or in any combination with IL-4 and/or IL-2,
particularly all embodiments, wherein the immunomodulatory substance(s) for use according to the present invention comprises, preferably is, IFN-γ or IFN-γ in combination with IL-4 and/or IL-2, and the immunomodulatory substance(s) administered in step (B) comprises, preferably is, IFN-γ or IFN-γ and IL-4,
the inflammatory disease, immunological disease and/or autoimmunological disease to be treated and/or prevented:
   comprises, preferably consists of,
   any of the inflammatory disease, immunological disease and/or autoimmunological disease to be treated and/or prevented as described herein except for an inflammatory disease of the nervous system, preferably except for multiple sclerosis;
   more preferably comprises, still preferably consists of,
   arthritis, synovitis, tenosynovitis, inflammatory rheumatic disease, Hashimoto's disease and/or Basedow's disease;
   even more preferably comprises, still even more preferably consists of,
   arthritis, synovitis, rheumatoid arthritis, psoriatic arthritis, polymyalgia rheumatica, Bechterew's disease and/or Basedow's disease.

Preferably, in all embodiments of the present invention described herein relating to BDNF, that is all embodiments mentioning BDNF alone or in combination with IL-4 and/or IL-2,
particularly all embodiments, wherein the immunomodulatory substance(s) for use according to the present invention comprises, preferably is, BDNF or BDNF in any combination with IL-4 and/or IL-2, and the immunomodulatory substance(s) administered in step (B) comprises, preferably is, BDNF or BDNF and IL-4,
the inflammatory disease, immunological disease and/or autoimmunological disease to be treated and/or prevented:
   comprises, preferably consists of,
   an inflammatory disease of the nervous system;
   more preferably comprises, even more preferably consists of,
   multiple sclerosis.

Preferably, in all embodiments of the present invention described herein relating to IL-4, that is all embodiments mentioning IL-4 alone or in combination with BDNF and/or IL-2,
particularly all embodiments, wherein the immunomodulatory substance(s) for use according to the present invention comprises, preferably is, IL-4 or IL-4 in any combination with BDNF and/or IL-2, and the immunomodulatory substance(s) administered in step (B) comprises, preferably is, IL-4 or IL-4 and BDNF,
the inflammatory disease, immunological disease and/or autoimmunological disease to be treated and/or prevented:
   comprises, preferably consists of,
   any of the inflammatory disease, immunological disease and/or autoimmunological disease to be treated and/or prevented as described herein;
   preferably comprises, more preferably consists of,
   an inflammatory disease of the nervous system;
   more preferably comprises, even more preferably consists of,
   multiple sclerosis.

Preferably, in all embodiments of the present invention described herein relating to IL-2, that is all embodiments mentioning IL-2 alone or in combination with IFN-γ, BDNF and/or IL-4,
particularly all embodiments, wherein the immunomodulatory substance(s) for use according to the present invention is any of the immunomodulatory substance(s) as described herein including IL-2 as disclosed herein, preferably is a cytokine(s) including IL-2, more preferably is an interferon(s), neurotrophin(s) and/or interleukin(s) including IL-2, even more preferably is IL-2 or IL-2 in any combination with IFN-γ, BDNF and/or IL-4, and the immunomodulatory substance(s) administered in step (C) comprises, preferably is, IL-2,
the inflammatory disease, immunological disease and/or autoimmunological disease to be treated and/or prevented:
   in case of IL-2,
   comprises, preferably consists of,
      the inflammatory disease, the immunological disease and/or autoimmunological disease as described herein;
   in case of IL-2 in any combination with IFN-γ and/or IL-4, preferably in combination with IFN-γ,
   preferably comprises, more preferably consists of,
      the inflammatory disease, immunological disease and/or autoimmunological disease, except for an inflammatory disease of the nervous system except for multiple sclerosis,
   more preferably comprises, more preferably consists of,
      arthritis, synovitis, tenosynovitis, inflammatory rheumatic disease, Hashimoto's disease and/or Basedow's disease, even more preferably comprises, still more preferably consists of,
      arthritis, synovitis, rheumatoid arthritis, psoriatic arthritis, polymyalgia rheumatica, Bechterew's disease and/or Basedow's disease;
   and/or
      in case of IL-2 in any combination with BDNF and/or IL-4,
      comprises, preferably consists of,
         an inflammatory disease of the nervous system,
      preferably comprises, preferably consists of,
         multiple sclerosis.

Hence, still even more preferably,
the method comprises steps (A) and (C);
the immunomodulatory substance(s) for use according to the present invention is any of the immunomodulatory substance(s) as described herein, that is the immunomodulatory substance(s) including IL-2, preferably a cytokine(s) including IL-2, more preferably an interferon(s), neurotrophin(s) and/or interleukin(s) including IL-2, even more preferably IL-2;
the inflammatory disease, immunological disease and/or autoimmunological disease to be treated and/or prevented is any of the inflammatory disease, immunological disease and/or autoimmunological disease to be treated and/or prevented as described herein; and
the immunomodulatory substance(s) in step (C) is IL-2,
or
the method comprises steps (A), (B) and (C);
the immunomodulatory substance(s) for use according to the present invention is any of the immunomodulatory substance(s) as described herein, that is the immunomodulatory substance(s) including IL-2, preferably a cytokine(s) including IL-2, more preferably an interferon(s), neurotrophin(s) and/or interleukin(s) including IL-2, even more preferably IL-2;
the inflammatory disease, immunological disease and/or autoimmunological disease to be treated and/or prevented is any of the inflammatory disease, immunological disease and/or autoimmunological disease to be treated and/or prevented as described herein;
the immunomodulatory substance(s) in step (B) is any of the immunomodulatory substance(s) as described herein except for IL-2; and
the immunomodulatory substance(s) in step (C) is IL-2,
or
the method comprises steps (A) and (B);
the immunomodulatory substance(s) for use according to the present invention is IFN-γ and/or IL-4, preferably IFN-γ;
the inflammatory disease, immunological disease and/or autoimmunological disease to be treated and/or prevented is any of the inflammatory disease, immunological disease and/or autoimmunological disease to be treated and/or prevented as described herein except for an inflammatory disease of the nervous system, preferably except for multiple sclerosis, preferably is arthritis, synovitis, tenosynovitis, inflammatory rheumatic disease, Hashimoto's disease and/or Basedow's disease, more preferably is arthritis, synovitis, rheumatoid arthritis, psoriatic arthritis, Bechterew's disease and/or Basedow's disease; and
the immunomodulatory substance(s) in step (B) is IFN-γ and/or IL-4, preferably is IFN-γ,
or
the method comprises steps (A) and (B);
the immunomodulatory substance(s) for use according to the present invention is IL-4 and/or BDNF, preferably IL-4; the inflammatory disease, immunological disease and/or autoimmunological disease to be treated and/or prevented is an inflammatory disease of the nervous system, preferably is multiple sclerosis; and the immunomodulatory substance(s) in step (B) is IL-4 and/or BDNF, preferably IL-4.

Furthermore preferably,
the method comprises steps (A), (B) and (C);
   the immunomodulatory substance(s) for use according to the present invention is IFN-γ and/or IL-2;
   the inflammatory disease, immunological disease and/or autoimmunological disease to be treated and/or prevented is any of the inflammatory disease, immunological disease and/or autoimmunological disease to be treated and/or prevented as described herein except for an inflammatory disease of the nervous system, preferably except for multiple sclerosis, preferably is arthritis, synovitis, tenosynovitis, inflammatory rheumatic disease, Hashimoto's disease and/or Basedow's disease, more preferably is arthritis, synovitis, rheumatoid arthritis, psoriatic arthritis, polymyalgia rheumatica, Bechterew's disease and/or Basedow's disease;
   the immunomodulatory substance(s) in step (B) is IFN-γ; and
   the immunomodulatory substance(s) in step (C) is IL-2,
or
   the immunomodulatory substance(s) for use according to the present invention is IL-4 and/or IL-2;
   the inflammatory disease, immunological disease and/or autoimmunological disease to be treated and/or prevented is any of the inflammatory disease, immunological disease and/or autoimmunological to be treated and/or prevented as described herein, preferably is arthritis, synovitis, tenosynovitis, inflammatory rheumatic disease, Hashimoto's disease Basedow's disease and/or multiple sclerosis, more preferably is arthritis, synovitis, rheumatoid arthritis, psoriatic arthritis, polymyalgia rheumatica, Bechterew's disease, Basedow's disease and/or multiple sclerosis;
   the immunomodulatory substance(s) in step (B) is IL-4; and
   the immunomodulatory substance(s) in step (C) is IL-2,
or the method comprises steps (A) and (B), wherein step (B) is performed a second time as (B₁);
   the immunomodulatory substance(s) for use according to the present invention is IFN-γ and/or IL-4;
   the inflammatory disease, immunological disease and/or autoimmunological disease to be treated and/or prevented is any of the inflammatory disease, immunological disease and/or autoimmunological disease to be treated and/or prevented as described herein except for an inflammatory disease of the nervous system, preferably except for multiple sclerosis, preferably is arthritis, synovitis, tenosynovitis, inflammatory rheumatic disease, Hashimoto's disease and/or Basedow's disease, more preferably is arthritis, synovitis, rheumatoid arthritis, psoriatic arthritis, polymyalgia rheumatica, Bechterew's disease and/or Basedow's disease;
   the immunomodulatory substance(s) in step (B) is IFN-γ; and
   the immunomodulatory substance(s) in step (B₁) is IL-4,
or
   the immunomodulatory substance(s) for use according to the present invention is IL-4 and/or BDNF;
   the inflammatory disease, immunological disease and/or autoimmunological disease to be treated and/or prevented is an inflammatory disease of the nervous system, preferably is multiple sclerosis;
   the immunomodulatory substance(s) in step (B) is IL-4; and
   the immunomodulatory substance(s) in step (B₁) is BDNF.

The embodiment, in which the method comprises steps (A), (B) and (C) is still more preferred.

Even furthermore preferably, the method comprises steps (A), (B), (B₁) and (C);
the immunomodulatory substance(s) for use according to the present invention is IFN-γ, IL-4 and/or IL-2;
the inflammatory disease, immunological disease and/or autoimmunological disease to be treated and/or prevented is any of the inflammatory disease, immunological disease and/or autoimmunological disease to be treated and/or prevented as described herein except for an inflammatory disease of the nervous system, preferably except for multiple sclerosis, preferably is arthritis, synovitis, tenosynovitis, inflammatory rheumatic disease, Hashimoto's disease and/or Basedow's disease, more preferably is arthritis, synovitis, rheumatoid arthritis, psoriatic arthritis, polymyalgia rheumatica, Bechterew's disease and/or Basedow's disease;
the immunomodulatory substance(s) in step (B) is IFN-γ;
the immunomodulatory substance(s) in step (B₁) is IL-4; and
the immunomodulatory substance(s) in step (C) is IL-2,
or
the immunomodulatory substance(s) for use according to the present invention is IL-4, BDNF and/or IL-2;
the inflammatory disease, immunological disease and/or autoimmunological disease to be treated and/or prevented is an inflammatory disease of the nervous system, preferably is multiple sclerosis;
the immunomodulatory substance(s) in step (B) is IL-4;
the immunomodulatory substance(s) in step (B₁) is BDNF; and the immunomodulatory substance(s) in step (C) is IL-2.

Still furthermore preferably,
the immunomodulatory substance(s) for use according to the present invention is IFN-γ, IL-4 and/or IL-2;
the inflammatory disease, immunological disease and/or autoimmunological disease to be treated and/or prevented is any of the inflammatory disease, immunological disease and/or autoimmunological disease to be treated and/or prevented as described herein except for an inflammatory disease of the nervous system, preferably except for multiple sclerosis, preferably is arthritis, synovitis, tenosynovitis, inflammatory rheumatic disease, Hashimoto's disease and/or Basedow's disease, more preferably is arthritis, synovitis, rheumatoid arthritis, psoriatic arthritis, polymyalgia rheumatica, Bechterew's disease and/or Basedow's disease;
and the method comprises:
a first set of steps, comprising:
(A) which is selected from one or more of steps (A-0), (A-1), (A-2), (A-3), (A-4), (A-5), (A-6), (A-7) and/or (A-8); and
(B) administering IFN-γ to the skin of said subject; and
(C) administering IL-2 to the skin of the subject, and
a second set of steps comprising:
(A) which is selected from one or more of steps (A-0), (A-1), (A-2), (A-3), (A-4), (A-5), (A-6), (A-7) and/or (A-8); and
(B₁) administering IL-4 to the skin of said subject; and
(C) administering IL-2 to the skin of the subject,
or
the immunomodulatory substance(s) for use according to the present invention is IL-4, BDNF and/or IL-2;
the inflammatory disease, immunological disease and/or autoimmunological disease to be treated and/or prevented is an inflammatory disease of the nervous system, preferably is multiple sclerosis;
and the method comprises
a first set of steps, comprising:
(A) which is selected from one or more of steps (A-0), (A-1), (A-2), (A-3), (A-4), (A-5), (A-6), (A-7) and/or (A-8);
and
(B) administering IL-4 to the skin of said subject; and
(C) administering IL-2 to the skin of the subject, and
a second set of steps comprising:
(A) which is selected from one or more of steps (A-0), (A-1), (A-2), (A-3), (A-4), (A-5), (A-6), (A-7) and/or (A-8); and
(B₁) administering BDNF to the skin of said subject; and
(C) administering IL-2 to the skin of the subject.

For the sake of simplicity and clarity, the above embodiments were only written out in full length for the cytokine(s), interferon(s), interleukin(s), neurotrophin(s), IFN-γ, IL-4, BDNF and/or IL-2. If not mentioned otherwise, it is to be understood that any of the preferred embodiments of the present invention stated herein for the cytokine(s), interferon(s), interleukin(s), neurotrophin(s), IFN-γ, IL-4, BDNF and/or IL-2 is independently and mutatis mutandis applicable to the cytokine-like acting substance(s), interferon-like acting substance(s), interleukin-like acting substance(s), neurotrophin-like acting substance(s), IFN-γ-like acting substance(s), IL-2-like acting substance(s), IL-4-like acting substance(s) and/or BDNF-like acting substance(s), wherein for that reason, where applicable, the term ‘cytokine(s)’ is to be understood ‘cytokine-like acting substance(s)’, ‘interferon(s)’ is to be understood ‘interferon-like acting substance(s)’, ‘interleukin(s)’. is to be understood ‘interleukin-like acting substance(s)’, ‘neurotrophin(s)’ is to be understood ‘neurotrophin-like acting substance(s)’, ‘IFN-γ’ is to be understood ‘IFN-γ-like acting substance(s)’, is to be understood ‘IL-4-like acting substance(s)’, ‘BDNF is to be understood ‘BDNF-like acting substance(s)’ and ‘IL-2’ is to be understood ‘IL-2-like acting substance(s)’. Preferably, in one particular embodiment all of such terms are replaced or all of such terms remain unchanged, however, not some replaced and some not. The embodiments referring to cytokine(s), interferon(s), neurotrophin(s), interleukin(s), IFN-γ, IL-4, BDNF, IL-2 and/or any derivative, fragment, protein-analogue, peptide, protein-variant, biopharmaceutical, inductor, precursor, prodrug, mutein, co-drug, propeptide thereof and/or any pharmaceutically acceptable salt of any of these are more preferred. The embodiments referring to cytokine(s), interferon(s), neurotrophin(s), interleukin(s), IFN-γ, IL-4, BDNF and/or IL-2 are even more preferred.

The expression “IFN-γ-like acting substance” or “IFN-γ-like acting substance(s)” as mentioned in any of the embodiments described herein can be, preferably is, any substance that exerts a similar or identical effect to a IFN-γ in the body of the subject when administered thereto. The IFN-γ-like acting substance(s) may encompass precursors, prodrugs and propeptides of such substances which are usually not active as such, i.e. have no immunomodulatory activity, but which, upon use in accordance with the present invention, are converted into the actually active IFN-γ-like acting substance(s). Hence, it may be any substance, molecule, peptide, protein, protein-analogue, protein-variant, derivative, fragment, biopharmaceutical, inductor, precursor, prodrug, mutein, co-drug and/or any derivative, fragment and/or pharmaceutically acceptable salt of any of these. Therefore IFN-γ-like acting substance(s) may:
activates or is/are capable of activating a respective IFN-γ-receptor(s) of a cell. The IFN-γ-receptor(s) may for instance be the receptor of the LHs, peripheral T_{reg}s or PBMCs; and/or
results in or causes or is capable of resulting in or causing the generation of a IFN-γ and/or a IFN-γ-like acting substance(s).

Preferably, the IFN-γ-like acting substance(s) activates or is capable of activating the respective IFN-γ-receptor(s). The activating or the capability of activating the respective IFN-γ-receptor(s) may be directly and/or it may be indirectly.

Preferably, the IFN-γ-like acting substance(s) activates or is/are capable of activating a respective IFN-γ-receptor(s) within the skin of the subject. The IFN-γ-like acting substance(s) may be any naturally occurring or artificial IFN-γ-like acting substance(s) as long as it has a sufficient biologic activity, i.e. an effective cross-reactivity in the subject, particularly is capable of activating the respective IFN-γ-receptor(s). Preferably, the IFN-γ-like acting substance(s) is known to the person skilled in the art at the effective date of filing of the present invention.

Preferably, the IFN-γ-like acting substance is any peptide, protein, protein-analogue, protein-variant or any derivative, fragment, biopharmaceutical, inductor, precursor, prodrug, mutein, co-drug and/or pharmaceutically acceptable salt of any of these.

In a preferred embodiment, the IFN-γ-like acting substance(s) may be as detailed below amongst others an IFN-γ and/or any derivative, fragment, protein-analogue, peptide, protein-variant, biopharmaceutical, inductor, precursor, prodrug, mutein, co-drug, propeptide thereof and/or any pharmaceutically acceptable salt of any of these.

More preferably, the IFN-γ-like acting substance(s) is an IFN-γ and/or any derivative, fragment, protein-analogue, peptide, protein-variant, biopharmaceutical, inductor, precursor, prodrug, mutein, co-drug, propeptide thereof and/or any pharmaceutically acceptable salt of any of these.

The expression “IFN-γ” as mentioned in any of the embodiments described herein preferably has to be interpreted broad. Typically, the IFN-γ activates or is are capable of activating a respective IFN-γ-receptor(s) of a cell. The IFN-γ-receptor(s) may for instance be the receptor of the LHs, peripheral T_{reg}s or PBMCs. Preferably, the activating or capability of activating is within the skin of the subject, more preferably locally in effective amounts within the skin of the subject, even more preferably not in amounts systemically effective within the subject's body. Preferably the IFN-γ may be any type of IFN-γ known to the person skilled in the art. Furthermore, the present invention encompass the use or medical use of precursors, prodrugs and propeptides of such IFN-γ which are usually not active as such, i.e. have no immunomodulatory activity, but which, upon use in accordance with the present invention, are converted into the actually active IFN-γ. Hence, these may be used in the method or the medical use as described herein or in preparation of the pharmaceutical compositions, injectable dosage form, topical dosage forms, medical devices and/or kit of parts as described herein.

Preferably, the IFN-γ may be any peptide, protein, protein-analogue, protein-variant or any derivative, fragment, biopharmaceutical, inductor, precursor, prodrug, mutein, co-drug and/or pharmaceutically acceptable salt of any of these.

The IFN-γ does not necessarily be derived from or be identical to the IFN-γ of the same genus and/or species the subject belongs to, it may be any naturally occurring or an artificial IFN-γ, as long as it has a sufficient biologic activity, i.e. an effective cross-reactivity in the subject, particularly is capable of activating the respective IFN-γ-receptor(s). Preferably, the biologic activity of the IFN-γ and/or IFN-γ-like acting substance(s) is sufficient to achieve the treating and/or preventing of the inflammatory disease, immunological disease and/or autoimmunological disease, preferably in the subject.

An IFN-γ and/or an IFN-γ-like acting substance typically affects cells by binding and activating the respective IFN-γ-receptor on the cells. Hence, the activating of the respective receptor typically leads to the affectation of the cell as described above for the immunomodulatory substance. Hence, any embodiment mentioned herein for the immunomodulatory substance relating to the affecting cells like LHs, peripheral T_{reg}s or PBMCs is independently and mutatis mutandis applicable to the IFN-γ and the IFN-γ-like acting substance.

The activating or the capability of activating the respective IFN-γ-receptor(s) by the IFN-γ and/or IFN-γ-like acting substance(s) may be directly and/or it may be indirectly.

If not mentioned otherwise, the description and definition of the cytokine(s) or the cytokine-like acting substance stated above is independently and mutatis mutandis applicable to the IFN-γ and/or IFN-γ-like acting substance(s). Particularly, if not mentioned otherwise, the description and definition of the cytokine(s) or the cytokine-like acting substance in respect of“directly” and “indirectly” activating or capable of activating the respective IFN-γ-receptor(s), is independently and mutatis mutandis applicable to the IFN-γ and/or IFN-γ-like acting substance(s).

Preferably, the IFN-γ and/or IFN-γ-like acting substance(s) for use according to the present invention and/or the IFN-γ and/or IFN-γ-like acting substance(s) of steps (B), (B₁) and/or (C) of the method, the pharmaceutical compositions, injectable dosage forms, topical dosage forms, medical devices and/or kits of parts, is capable of affecting and/or affects the LHs, peripheral T_{reg}s or PBMCs. Preferably, the PBMCs are naïve PBMCs, more preferably lymphocytes, even more preferably T-cells and/or B-cells, still more preferably naïve lymphocytes, still even more preferably naïve B-cells and/or naïve T-cells, further preferably naïve T-cells.

More preferably, the IFN-γ and/or IFN-γ-like acting substance(s) is capable of directly and/or indirectly affecting and/or affects the LHs, peripheral T_{reg}s or PBMCs.

More preferably, the IFN-γ and/or IFN-γ-like acting substance(s) is capable of directly and/or indirectly affecting and/or affects the LHs, peripheral T_{reg}s or PBMCs in the subject as defined in any of the embodiments according to the present invention, further preferably within the skin of the subject.

If not mentioned otherwise, the description and definition of the immunomodulatory substance(s) stated above is independently and mutatis mutandis applicable to the IFN-γ and/or IFN-γ-like acting substance(s). Particularly, if not mentioned otherwise, the description and definition of the immunomodulatory substance(s) in respect of “directly” and “indirectly” affecting or capable of affecting the PBMCs is independently and mutatis mutandis applicable to the IFN-γ and/or IFN-γ-like acting substance(s).

The IFN-γ and/or IFN-γ-like acting substance(s) for use according to the present invention and/or the IFN-γ and/or IFN-γ-like acting substance(s) of steps (B), (B₁) and/or (C) of the method, the pharmaceutical compositions, injectable dosage forms, topical dosage forms, medical devices and/or kits of parts, may be similar or identical, preferably identical, to any IFN-γ and/or IFN-γ-like acting substance(s) naturally occurring in a vertebrate, preferably a human or a mammal, more preferably in the subject as defined in any of the embodiments according to the present invention. Preferably, the IFN-γ and/or IFN-γ-like acting substance(s) may be similar or identical, preferably identical, to any peptide, protein or any fragment of these of IFN-γ and/or IFN-γ-like acting substance(s) naturally occurring in a vertebrate, preferably a human or a mammal as defined above, more preferably in the subject as defined in any of the embodiments according to the present invention.

The IFN-γ and/or IFN-γ-like acting substance(s) may comprise, preferably consist of an amino acid sequence which has an amino acid sequence identity to any peptide, protein or any fragment of an IFN-γ and/or IFN-γ-like acting substance(s) naturally occurring in a vertebrate, preferably a mammal, more preferably a human or a mammal animal as defined above, more preferably in the subject as defined in any of the embodiments according to the present invention. More preferably, the amino acid sequence identity is with reference to amino acid sequence SEQ ID NO: 1 or any fragment thereof. Preferably, the amino acid sequence identity is 70% or more, even more preferably 85% or more, still more preferably 90% or more, still even more preferably 95% or more, further preferably 97% or more, even further preferably 98% or more, still further preferably 99% or more and 100% or less. Preferably, the amino acid sequence identity includes substitutions, insertions and/or deletions of amino acid residues in respect to the naturally occurring peptide, protein or any fragment of these.

Preferably, the total number of substitutions, insertions and/or deletions or the total number combined of substitutions, insertions and/or deletions of amino acid residues is 60 or less, more preferably 40 or less, even more preferably 20 or less, still more preferably 12 or less, still even more preferably 8 or less, further preferably 4 or less, even further preferably 2 or less and zero or more, still further preferably zero, preferably with reference to amino acid sequence SEQ ID NO: 1, wherein more preferably the subject is a human.

The IFN-γ and/or IFN-γ-like acting substance(s) may be identical to an endogenous immunomodulatory substance(s) of the same genus and/or species the subject belongs to or the amino acid sequence identity refers to an endogenous immunomodulatory substance(s) of the same genus and/or species the subject belongs to. The expression “is identical” typically means in this particular context that the IFN-γ and/or IFN-γ-like acting substance(s) is in respect to its primary, secondary, tertiary and quaternary protein structure identical to an/the IFN-γ and/or IFN-γ-like acting substance(s) of the same genus and/or species the subject belongs to, more preferably, also in view of the modification pattern, particularly glycosylations.

The IFN-γ and/or IFN-γ-like acting substance(s) for use according to the present invention and/or the IFN-γ and/or IFN-γ-like acting substance(s) of steps (B), (B₁) and/or (C) of the method, the pharmaceutical compositions, injectable dosage forms, topical dosage forms, medical devices and/or kits of parts, may be derived from an individual belonging to the same genus and/or species as the subject, or which is identical to an/the IFN-γ and/or IFN-γ-like acting substance(s) of the same genus and/or species the subject belongs to. For instance, in case the subject is: a human the IFN-γ and/or IFN-γ-like acting substance(s) is human IFN-γ and/or IFN-γ-like acting substance(s), more preferably human IFN-γ and/or IFN-γ-like acting substance(s) comprising, even more preferably consisting of, preferably as effective amino acid sequence, the amino acid sequence SEQ ID NO: 1; a cow the IFN-γ and/or IFN-γ-like acting substance(s) is bovine IFN-γ and/or IFN-γ-like acting substance(s); a horse the IFN-γ and/or IFN-γ-like acting substance(s) is equine IFN-γ and/or IFN-γ-like acting substance(s); a donkey the IFN-γ and/or IFN-γ-like acting substance(s) is donkey IFN-γ and/or IFN-γ-like acting substance(s); an elephant the IFN-γ and/or IFN-γ-like acting substance(s) is elephant IFN-γ and/or IFN-γ-like acting substance(s); a sheep the IFN-γ and/or IFN-γ-like acting substance(s) is sheep IFN-γ and/or IFN-γ-like acting substance(s); a goat the IFN-γ and/or IFN-γ-like acting substance(s) is goat IFN-γ and/or IFN-γ-like acting substance(s); a pig the IFN-γ and/or IFN-γ-like acting substance(s) is porcine IFN-γ and/or IFN-γ-like acting substance(s); a rabbit the IFN-γ and/or IFN-γ-like acting substance(s) is rabbit IFN-γ and/or IFN-γ-like acting substance(s); a mouse the IFN-γ and/or IFN-γ-like acting substance(s) is mouse IFN-γ and/or IFN-γ-like acting substance(s); a rat the IFN-γ and/or IFN-γ-like acting substance(s) is rat IFN-γ and/or IFN-γ-like acting substance(s); a camel the IFN-γ and/or IFN-γ-like acting substance(s) is camel IFN-γ and/or IFN-γ-like acting substance(s); a dromedary the IFN-γ and/or IFN-γ-like acting substance(s) is dromedary IFN-γ and/or IFN-γ-like acting substance(s); a lama the IFN-γ and/or IFN-γ-like acting substance(s) is lama IFN-γ and/or IFN-γ-like acting substance(s); an alpaca the IFN-γ and/or IFN-γ-like acting substance(s) is alpaca IFN-γ and/or IFN-γ-like acting substance(s); a dog the IFN-γ and/or IFN-γ-like acting substance(s) is dog IFN-γ and/or IFN-γ-like acting substance(s); and/or a cat the IFN-γ and/or IFN-γ-like acting substance(s) is cat IFN-γ and/or IFN-γ-like acting substance(s).

Preferably, the IFN-γ and/or IFN-γ-like acting substance(s) for use according to the present invention and/or the IFN-γ and/or IFN-γ-like acting substance(s) of steps (B), (B₁) and/or (C) of the method, the pharmaceutical compositions, injectable dosage forms, topical dosage forms, medical devices and/or kits of parts, is not the subject's endogenous IFN-γ and/or IFN-γ-like acting substance(s). In other words, preferably the IFN-γ and/or IFN-γ-like acting substance(s) is not an IFN-γ and/or IFN-γ-like acting substance(s) isolated from the subject's body, i.e. from the individual subject to be treated or prevented.

Preferably, the IFN-γ and/or IFN-γ-like acting substance(s) for use according to the present invention and/or the IFN-γ and/or IFN-γ-like acting substance(s) of steps (B), (B₁) and/or (C) of the method, the pharmaceutical compositions, injectable dosage forms, topical dosage forms, medical devices and/or kits of parts, may be, preferably, is a recombinant IFN-γ and/or IFN-γ-like acting substance(s) which may be generated by any recombinant expression techniques known to the person skilled in the art, a chemically synthesized IFN-γ and/or IFN-γ-like acting substance(s) synthesized by any production techniques known to the person skilled in the art, an artificially produced IFN-γ and/or IFN-γ-like acting substance(s) produced by any production techniques known to the person skilled in the art, a naturally occurring IFN-γ and/or IFN-γ-like acting substance(s) obtained from natural sources like an animal or human, or any combination thereof. More preferably, the IFN-γ and/or IFN-γ-like acting substance(s) is a recombinant IFN-γ and/or IFN-γ-like acting substance(s), chemically synthesized IFN-γ and/or IFN-γ-like acting substance(s), artificially produced IFN-γ and/or IFN-γ-like acting substance(s) or any combination thereof, even more preferably a recombinant IFN-γ and/or IFN-γ-like acting substance(s), still even more preferably recombinant human IFN-γ-1b-protein (IFN-γ gamma-1b) preferably produced in genetically modified Escherichia coli (produced by Boehringer Ingelheim RCV GmbH & Co KG and in Germany commercially available from Boehringer Ingelheim Pharma GmbH & Co. KG under the tradename Imukin®, PZN: 06958744, Lot: M000487).

Furthermore, the IFN-γ and/or IFN-γ-like acting substance(s) for use according to the present invention and/or the IFN-γ and/or IFN-γ-like acting substance(s) of steps (B), (B₁) and/or (C) of the method, the pharmaceutical compositions, injectable dosage forms, topical dosage forms, medical devices and/or kits of parts, may or may not be derivatized, stabilized, fused with other proteins or peptides, conjugated with polymers, contain amino acid analogue(s) or artificial amino acids, modified, covalently or non-covalently, for instance glycosylated or methylated by e.g. posttranslational modification and/or oligomerized, e.g. dimerized or trimerized as long as it is capable of affecting and/or affects LHs, peripheral T_{reg}s or PBMCs. More preferably, the modification pattern and/or oligomerization status is similar or identical to the peptide, protein or any fragment of these naturally occurring in a vertebrate, preferably a mammal, more preferably a human or a mammal animal as defined above, more preferably in the subject as defined in any of the embodiments according to the present invention.

Preferably, in any of the embodiments described herein it is intended to administer the IFN-γ and/or IFN-γ-like acting substance(s) in an amount low enough to avoid a systemic increase of the IFN-γ and/or IFN-γ-like acting substance(s)-concentration in the subject's body. Hence, the IFN-γ and/or IFN-γ-like acting substance(s) may be administered in an amount that, when scaled to the entire body of the subject, the resulting systemic concentrations remain in the magnitude of the endogenous levels. Thereby, without wishing to be bound to theory, it is believed that the generation of an increased IFN-γ and/or IFN-γ-like acting substance(s)-concentration for instance at sites of inflammation, like inflamed joint. As already stated above, for instance naïve T-cells can mature in the presence of antigen/autoantigen/allergen towards cytotoxic, which may act pro-inflammatory. Thereby decreasing, cancelling or even reversing the beneficial effects achieved by the present invention, particularly, the anti-inflammatory effect of the regulatory T-cells and/or helper T-cells, or of a subset thereof, which are presumably generated.

Preferably, in any of the embodiments described herein, particularly in step (B), (B₁) and/or (C), the IFN-γ and/or IFN-γ-like acting substance(s) is administered in an amount that does not cause a systemic increase, more preferably an effective systemic increase, of the concentration of IFN-γ and/or IFN-γ-like acting substance(s) in the subject, preferably the blood of the subject, and/or that causes a systemic increase of the concentration of the IFN-γ and/or IFN-γ-like acting substance(s). Preferably, IFN-γ and/or IFN-γ-like acting substance(s) is administered in an amount low enough that the systemic increase is non-effective.

Preferably, in any of the embodiments described herein, particularly in step (B), (B₁) and/or (C), the IFN-γ and/or IFN-γ-like acting substance(s) is administered in an amount that causes a local increase, preferably an effective local increase, of the concentration of the IFN-γ and/or IFN-γ-like acting substance(s) in the subject, preferably in the skin tissue of the subject. The skin tissue is preferred, because unlike administration into the blood, no or less flushing away and dilution of the IFN-γ and/or IFN-γ-like acting substance(s) takes place.

Preferably, in any of the embodiments described herein, particularly in step (B), (B₁) and/or (C), the generated increased concentration, preferably the generated effective increased concentration of the IFN-γ and/or IFN-γ-like acting substance(s) administered is only local but not systemic in the subject.

Preferably, in any of the embodiments described herein, particularly in step (B), (B₁) and/or (C), the IFN-γ and/or IFN-γ-like acting substance(s) is administered in an amount that does not cause a systemic activation, preferably an effective systemic activation, of the respective receptor of the IFN-γ and/or IFN-γ-like acting substance(s) of a cell, and/or that does not cause a systemic generation, preferably an effective systemic generation, of the IFN-γ and/or IFN-γ-like acting substance(s), preferably in the subject.

Preferably, in any of the embodiments described herein, particularly in step (B), (B₁) and/or (C), the IFN-γ and/or IFN-γ-like acting substance(s) is administered in an amount that causes a local activation, preferably an effective local activation of the respective receptor of the IFN-γ and/or IFN-γ-like acting substance(s) of a cell, and/or causes a local generation, preferably an effective local generation of the IFN-γ and/or IFN-γ-like acting substance(s), preferably in the subject. Preferably, the activation and/or generation, more preferably the effective generation and/or effective activation is only local but not systemic in the subject.

Generally, 1 ng (nanogram(s)) IFN-γ corresponds to approximately 20 IU (international unit(s)) IFN-γ or less. Some of the IFN-γ might be denatured or otherwise defect in that the biological activity of IFN-γ has been lost while the mass remains constant.

Preferably, in any of the embodiments described herein, particularly in step (B), (B₁) and/or (C), the IFN-γ-like acting substance(s), preferably the IFN-γ, is administered in an effective amount, preferably an effective total amount.

Preferably, in any of the embodiments described herein, particularly in step (B), (B₁) and/or (C), the IFN-γ-like acting IFN-γ substance(s) is administered in an amount, preferably a total amount, equivalent to 600 IU IFN-γ/kg (international unit(s)/kilogram) body mass of the subject or less (‘kg body mass’ is in its meaning IFN-γ equivalent to ‘kg of the body mass’), preferably per administration dose, to the skin of the subject. More preferably, IFN-γ is administered in an amount, preferably a total amount, equivalent to 300 IU IFN-γ/kg or less, even more preferably 200 IU IFN-γ/kg or less, still more preferably 100 IU IFN-γ/kg or less, still even more preferably 50 IU IFN-γ/kg or less, further preferably 30 IU IFN-γ/kg or less, further preferably 20 IU IFN-γ/kg or less, even further preferably 6 IU IFN-γ/kg or less. There is no lower limit for the amount per kg body mass as long IFN-γ as the beneficial effects can be achieved, however, usually, a lower limit is and amount equivalent to 0.04 IU IFN-γ/kg or more, more preferably 0.2 IU IFN-γ/kg or more, even more preferably, 0.5 IU IFN-γ/kg or more, still more preferably 1 IU IFN-γ/kg or more, still even more preferably 1.5 IU IFN-γ/kg or more. More preferably, the IFN-γ-like acting IFN-γ substance(s) is administered in an amount, preferably a total amount, in the rang IFN-γe of equivalent to 0.04 IU IFN-γ/kg or more and 600 IU IFN-γ/kg or less, even more preferably 0.2 IU IFN-γ/kg or more and 300 IU IFN-γ/kg or less, still more preferably 0.5 IU IFN-γ/kg or more and 200 IU IFN-γ/kg or less, still even more preferably 0.5 IU IFN-γ/kg or more and 100 IU IFN-γ/kg or less, further preferably 0.5 IU IFN-γ/kg or more and 50 IU IFN-γ/kg or less, even further preferably 1 IU IFN-γ/kg or more and 30 IU IFN-γ/kg or less, still further preferably 1.5 IU IFN-γ/kg or more and 20 IU IFN-γ/kg or less.

Preferably, in any of the embodiments described herein, particularly in step (B), (B₁) and/or (C), the IFN-γ-like acting IFN-γ substance(s) is administered in an amount, preferably a total amount, equivalent in activity to 30 ng IFN-γ/kg body mass of the subject or less, preferably per administration dose, to the skin of the subject. More preferably, IFN-γ-like aktinv substance is administered in an amount, preferably a total amount, equivalent in activity to 15 ng IFN-γ/kg or less, even more preferably 10 ng IFN-γ/kg or less, still more preferably 5 ng IFN-γ/kg or less, still even more preferably 2.5 ng IFN-γ/kg or less, further preferably 1.5 ng IFN-γ/kg or less, still even more preferably 1 ng IFN-γ/kg or less, even further preferably 0.3 ng IFN-γ/kg or less. There is no lower limit for the amount per kg body mass as long IFN-γ as the beneficial effects can be achieved, however, usually, a lower limit is an amount equivalent in activity to 0.002 ng IFN-γ/kg or more, more preferably 0.025 ng IFN-γ/kg or more, even more preferably 0.01 ng IFN-γ/kg or more, still more preferably 0.02 mg/kg or more, still even more preferably 0.05 ng IFN-γ/kg or more, further preferably 0.075 ng IFN-γ/kg or more. More preferably, the IFN-γ-like acting IFN-γ substance(s) is administered in an amount, preferably a total amount, in the rang IFN-γe of equivalent in activity to 30 ng IFN-γ/kg or less and 0.002 ng IFN-γ/kg or more, even more preferably ng IFN-γ/kg or less and 0.025 ng IFN-γ/kg or more, still more preferably 10 ng IFN-γ/kg or less and 0.01 ng IFN-γ/kg or more, still even more preferably 5 ng IFN-γ/kg or less and 0.01 ng IFN-γ/kg or more, further preferably 2.5 ng IFN-γ/kg or less and 0.02 ng IFN-γ/kg or more, even further preferably 1.5 ng IFN-γ/kg or less and 0.02 ng IFN-γ/kg or more, still further preferably 1 ng IFN-γ/kg or less and 0.05 ng IFN-γ/kg or more, still even further preferably 0.3 ng IFN-γ/kg or less and 0.075 ng IFN-γ/kg or more.

Preferably, in any of the embodiments described herein, particularly in step (B), (B₁) and/or (C), the IFN-γ-like acting IFN-γ substance(s), is administered, preferably per administration dose, in an amount, preferably a total amount, equivalent to 30,000 IU IFN-γ or less, more preferably 15,000 IU IFN-γ or less, even more preferably 10,000 IU IFN-γ or less, still more preferably 5,000 IU IFN-γ or less, still even more preferably 2,500 IU IFN-γ or less, further preferably 1,500 IU IFN-γ or less, still further preferably 1,000 IU IFN-γ or less, even further preferably 300 IU IFN-γ or less. There is no lower limit for the amount as long IFN-γ as the beneficial effects can be achieved, however, usually, a lower limit is and amount equivalent to 2 IU IFN-γ or more, more preferably 10 IU IFN-γ or more, even more preferably 20 IU IFN-γ or more, still more preferably 30 IU IFN-γ or more and still even more preferably 50 IU IFN-γ or more. More preferably, the IFN-γ-like acting IFN-γ substance(s), is administered, preferably per administration dose, in an amount, preferably a total amount, in the rang IFN-γe of equivalent to 30,000 IU IFN-γ or less and 2 IU IFN-γ or more, even more preferably 15,000 IU IFN-γ or less and 2 IU IFN-γ or more, still more preferably 10,000 IU IFN-γ or less and IU IFN-γ or more, still even more preferably 5,000 IU IFN-γ or less and 10 IU IFN-γ or more, further preferably 2,500 IU IFN-γ or less and 20 IU IFN-γ or more, even further preferably 1,500 IU IFN-γ or less and 30 IU IFN-γ or more, still further preferably 1,000 IU IFN-γ or less and 50 IU IFN-γ or more, still even further preferably 300 IU IFN-γ or less and 50 IU IFN-γ or more.

Preferably, in any of the embodiments described herein, particularly in step (B), (B₁) and/or (C), the IFN-γ-like acting IFN-γ substance(s), is administered, preferably per administration dose, in an amount, preferably a total amount, equivalent in activity to 1,500 ng IFN-γ or less, more preferably 750 ng IFN-γ or less, even more preferably 500 ng IFN-γ or less, still more preferably 250 ng IFN-γ or less, still even more preferably 125 ng IFN-γ or less, further preferably 75 ng IFN-γ or less, still further preferably 50 ng IFN-γ or less, even further preferably 15 ng IFN-γ or less. There is no lower limit for the amount, preferably the total amount, as long IFN-γ as the beneficial effects can be achieved, however, usually, a lower limit is an amount equivalent in activity to 0.1 ng IFN-γ or more, more preferably 0.5 ng IFN-γ or more, even more preferably 1 ng IFN-γ or more and still even more preferably 2 ng IFN-γ or more. More preferably, the IFN-γ-like acting IFN-γ substance(s), is administered, preferably per administration dose, in an amount, preferably a total amount, in the rang IFN-γe of equivalent in activity to 1,500 ng IFN-γ or less and 0.1 ng IFN-γ or more, even more preferably 750 ng IFN-γ or less and 0.1 ng IFN-γ or more, still more preferably 500 ng IFN-γ or less and 0.1 ng IFN-γ or more, still even more preferably 250 ng IFN-γ or less and 0.5 ng IFN-γ or more, further preferably 125 ng IFN-γ or less and 0.5 ng IFN-γ or more, even further preferably 75 ng IFN-γ or less and 1 ng IFN-γ or more, still further preferably 50 ng IFN-γ or less and 1 ng IFN-γ or more, still even further preferably 15 ng IFN-γ or less and 2 ng IFN-γ or more.

Preferably, in any of the embodiments described herein, particularly in step (B), (B₁) and/or (C), IFN-γ is administered in an amount, preferably a total amount, of 600 IU/kg (international unit(s)/kilogram) body mass of the subject or less (‘kg body mass’ is in its meaning equivalent to ‘kg of the body mass’), preferably per administration dose, to the skin of the subject. More preferably, IFN-γ is administered in an amount, preferably a total amount, of 300 IU/kg or less, even more preferably 200 IU/kg or less, still more preferably 100 IU/kg or less, still even more preferably 50 IU/kg or less, further preferably 30 IU/kg or less, further preferably 20 IU/kg or less, even further preferably 6 IU/kg or less. There is no lower limit for the amount per kg body mass as long as the beneficial effects can be achieved, however, usually, a lower limit is 0.04 IU/kg or more, more preferably 0.2 IU/kg or more, even more preferably, 0.5 IU/kg or more, still more preferably 1 IU/kg or more, still even more preferably 1.5 IU/kg or more. More preferably, IFN-γ is administered in an amount, preferably a total amount, in the range of 0.04 IU/kg or more and 600 IU/kg or less, even more preferably 0.2 IU/kg or more and 300 IU/kg or less, still more preferably 0.5 IU/kg or more and 200 IU/kg or less, still even more preferably 0.5 IU/kg or more and 100 IU/kg or less, further preferably 0.5 IU/kg or more and 50 IU/kg or less, even further preferably 1 IU/kg or more and 30 IU/kg or less, still further preferably 1.5 IU/kg or more and 20 IU/kg or less.

Preferably, in any of the embodiments described herein, particularly in step (B), (B₁) and/or (C), IFN-γ is administered in an amount, preferably a total amount, of 30 ng/kg body mass of the subject or less, preferably per administration dose, to the skin of the subject. More preferably, IFN-γ is administered in an amount, preferably a total amount, of 15 ng/kg or less, even more preferably 10 ng/kg or less, still more preferably 5 ng/kg or less, still even more preferably 2.5 ng/kg or less, further preferably 1.5 ng/kg or less, still even more preferably 1 ng/kg or less, even further preferably 0.3 ng/kg or less. There is no lower limit for the amount per kg body mass as long as the beneficial effects can be achieved, however, usually, a lower limit is 0.002 ng/kg or more, more preferably 0.025 ng/kg or more, even more preferably 0.01 ng/kg or more, still more preferably 0.02 mg/kg or more, still even more preferably 0.05 ng/kg or more, further preferably 0.075 ng/kg or more. More preferably, IFN-γ is administered in an amount, preferably a total amount, in the range of 30 ng/kg or less and 0.002 ng/kg or more, even more preferably 15 ng/kg or less and 0.025 ng/kg or more, still more preferably ng/kg or less and 0.01 ng/kg or more, still even more preferably 5 ng/kg or less and 0.01 ng/kg or more, further preferably 2.5 ng/kg or less and 0.02 ng/kg or more, even further preferably 1.5 ng/kg or less and 0.02 ng/kg or more, still further preferably 1 ng/kg or less and 0.05 ng/kg or more, still even further preferably 0.3 ng/kg or less and 0.075 ng/kg or more.

Preferably, in any of the embodiments described herein, particularly in step (B), (B₁) and/or (C), the IFN-γ is administered, preferably per administration dose, in an amount, preferably a total amount, of 30,000 IU or less, more preferably 15,000 IU or less, even more preferably 10,000 IU or less, still more preferably 5,000 IU or less, still even more preferably 2,500 IU or less, further preferably 1,500 IU or less, still further preferably 1,000 IU or less, even further preferably 300 IU or less. There is no lower limit for the amount as long as the beneficial effects can be achieved, however, usually, a lower limit is 2 IU or more, more preferably 10 IU or more, even more preferably 20 IU or more, still more preferably 30 IU or more and still even more preferably 50 IU or more. More preferably, IFN-γ is administered, preferably per administration dose, in an amount, preferably a total amount, in the range of 30,000 IU or less and 2 IU or more, even more preferably 15,000 IU or less and 2 IU or more, still more preferably 10,000 IU or less and 10 IU or more, still even more preferably 5,000 IU or less and 10 IU or more, further preferably 2,500 IU or less and 20 IU or more, even further preferably 1,500 IU or less and 30 IU or more, still further preferably 1,000 IU or less and 50 IU or more, still even further preferably 300 IU or less and 50 IU or more.

Preferably, in any of the embodiments described herein, particularly in step (B), (B₁) and/or (C), the IFN-γ, preferably IFN-γ is administered, preferably per administration dose, in an amount, preferably a total amount, of 1,500 ng or less, more preferably 750 ng or less, even more preferably 500 ng or less, still more preferably 250 ng or less, still even more preferably 125 ng or less, further preferably 75 ng or less, still further preferably 50 ng or less, even further preferably 15 ng or less. There is no lower limit for the amount, preferably the total amount, as long as the beneficial effects can be achieved, however, usually, a lower limit is 0.1 ng or more, more preferably 0.5 ng or more, even more preferably 1 ng or more and still even more preferably 2 ng or more. More preferably, IFN-γ is administered, preferably per administration dose, in an amount, preferably a total amount, in the range of 1,500 ng or less and 0.1 ng or more, even more preferably 750 ng or less and 0.1 ng or more, still more preferably 500 ng or less and 0.1 ng or more, still even more preferably 250 ng or less and 0.5 ng or more, further preferably 125 ng or less and 0.5 ng or more, even further preferably 75 ng or less and 1 ng or more, still further preferably 50 ng or less and 1 ng or more, still even further preferably 15 ng or less and 2 ng or more.

IFN-γ-like acting substances like IFN-γ may be amongst others capable to effect the desired affectation of the LHs, peripheral T_{reg}s or PBMCs as described above.

As can be seen from the examples that IFN-γ has amongst others the effect of e.g. decreasing the CRP amounts, reducing joint-swelling, reducing pressure sensitivity of joints, reducing pain, reducing fatigue and improving life quality. The CRP-value is indicative for the level of inflammation. A higher CRP-value indicates a stronger inflammation, a CPR of 1 or below is considered healthy. The decrease in the CRP-value conferred by IFN-γ may even be into the healthy CRP-range of 5 mg/l (milligram/litre) or less or even 1 mg/l or less. Furthermore, IFN-γ decreases the BASDAI-score and the HAQ-score by several score points. High scores indicate a worse condition of the subject whereas a lower score indicates an improved condition.

The expression “IL-2-like acting substance” or “IL-2-like acting substance(s)” as mentioned in any of the embodiments described herein can be, preferably is, any substance that exerts a similar or identical effect to a IL-2 in the body of the subject when administered thereto. The IL-2-like acting substance(s) may encompass precursors, prodrugs and propeptides of such substances which are usually not active as such, i.e. have no immunomodulatory activity, but which, upon use in accordance with the present invention, are converted into the actually active IL-2-like acting substance(s). Hence, it may be any substance, molecule, peptide, protein, protein-analogue, protein-variant, derivative, fragment, biopharmaceutical, inductor, precursor, prodrug, mutein, co-drug and/or any derivative, fragment and/or pharmaceutically acceptable salt of any of these. Therefore IL-2-like acting substance(s) may:
activates or is/are capable of activating a respective IL-2-receptor(s) of a cell. The IL-2-receptor(s) may for instance be the receptor of the LHs, peripheral T_{reg}s or PBMCs; and/or
results in or causes or is capable of resulting in or causing the generation of a IL-2 and/or a IL-2-like acting substance(s).

Preferably, the IL-2-like acting substance(s) activates or is capable of activating the respective IL-2-receptor(s). The activating or the capability of activating the respective IL-2-receptor(s) may be directly and/or it may be indirectly.

Preferably, the IL-2-like acting substance(s) activates or is/are capable of activating a respective IL-2-receptor(s) within the skin of the subject. The IL-2-like acting substance(s) may be any naturally occurring or artificial IL-2-like acting substance(s) as long as it has a sufficient biologic activity, i.e. an effective cross-reactivity in the subject, particularly is capable of activating the respective IL-2-receptor(s). Preferably, the IL-2-like acting substance(s) is known to the person skilled in the art at the effective date of filing of the present invention.

Preferably, the IL-2-like acting substance is any peptide, protein, protein-analogue, protein-variant or any derivative, fragment, biopharmaceutical, inductor, precursor, prodrug, mutein, co-drug and/or pharmaceutically acceptable salt of any of these.

In a preferred embodiment, the IL-2-like acting substance(s) may be as detailed below amongst others an IL-2 and/or any derivative, fragment, protein-analogue, peptide, protein-variant, biopharmaceutical, inductor, precursor, prodrug, mutein, co-drug, propeptide thereof and/or any pharmaceutically acceptable salt of any of these.

More preferably, the IL-2-like acting substance(s) is an IL-2 and/or any derivative, fragment, protein-analogue, peptide, protein-variant, biopharmaceutical, inductor, precursor, prodrug, mutein, co-drug, propeptide thereof and/or any pharmaceutically acceptable salt of any of these.

The expression “IL-2” as mentioned in any of the embodiments described herein preferably has to be interpreted broad. Typically, the IL-2 activates or is are capable of activating a respective IL-2-receptor(s) of a cell. The IL-2-receptor(s) may for instance be the receptor of the LHs, peripheral T_{reg}s or PBMCs. Preferably, the activating or capability of activating is within the skin of the subject, more preferably locally in effective amounts within the skin of the subject, even more preferably not in amounts systemically effective within the subject's body. Preferably the IL-2 may be any type of IL-2 known to the person skilled in the art. Furthermore, the present invention encompass the use or medical use of precursors, prodrugs and propeptides of such IL-2 which are usually not active as such, i.e. have no immunomodulatory activity, but which, upon use in accordance with the present invention, are converted into the actually active IL-2. Hence, these may be used in the method or the medical use as described herein or in preparation of the pharmaceutical compositions, injectable dosage form, topical dosage forms, medical devices and/or kit of parts as described herein.

The IL-2 may be any peptide, protein, protein-analogue, protein-variant or any derivative, fragment, biopharmaceutical, inductor, precursor, prodrug, mutein, co-drug and/or pharmaceutically acceptable salt of any of these.

The IL-2 does not necessarily be derived from or be identical to the IL-2 of the same genus and/or species the subject belongs to, it may be any naturally occurring or an artificial IL-2, as long as it has a sufficient biologic activity, i.e. an effective cross-reactivity in the subject, particularly is capable of activating the respective IL-2-receptor(s). Preferably, the biologic activity of the IL-2 and/or IL-2-like acting substance(s) is sufficient to achieve the treating and/or preventing of the inflammatory disease, immunological disease and/or autoimmunological disease, preferably in the subject.

An IL-2 and/or an IL-2-like acting substance typically affects cells by binding and activating the respective IL-2-receptor on the cells. Hence, the activating of the respective receptor typically leads to the affectation of the cell as described above for the immunomodulatory substance. Hence, any embodiment mentioned herein for the immunomodulatory substance relating to the affecting cells like LHs, peripheral T_{reg}s or PBMCs is independently and mutatis mutandis applicable to the IL-2 and the IL-2-like acting substance.

The activating or the capability of activating the respective IL-2-receptor(s) by the IL-2 and/or IL-2-like acting substance(s) may be directly and/or it may be indirectly.

If not mentioned otherwise, the description and definition of the cytokine(s) or the cytokine-like acting substance stated above is independently and mutatis mutandis applicable to the IL-2 and/or IL-2-like acting substance(s). Particularly, if not mentioned otherwise, the description and definition of the cytokine(s) or the cytokine-like acting substance in respect of “directly” and “indirectly” activating or capable of activating the respective IL-2-receptor(s), is independently and mutatis mutandis applicable to the IL-2 and/or IL-2-like acting substance(s).

Preferably, the IL-2 and/or IL-2-like acting substance(s) for use according to the present invention and/or the IL-2 and/or IL-2-like acting substance(s) of steps (B), (B₁) and/or (C) of the method, the pharmaceutical compositions, injectable dosage forms, topical dosage forms, medical devices and/or kits of parts, is capable of affecting and/or affects the LHs, peripheral T_{reg}s or PBMCs. Preferably, the PBMCs are naïve PBMCs, more preferably lymphocytes, even more preferably T-cells and/or B-cells, still more preferably naïve lymphocytes, still even more preferably naïve B-cells and/or naïve T-cells, further preferably naïve T-cells.

More preferably, the IL-2 and/or IL-2-like acting substance(s) is capable of directly and/or indirectly affecting and/or affects the LHs, peripheral T_{reg}s or PBMCs.

More preferably, the IL-2 and/or IL-2-like acting substance(s) is capable of directly and/or indirectly affecting and/or affects the LHs, peripheral T_{reg}s or PBMCs in the subject as defined in any of the embodiments according to the present invention, further preferably within the skin of the subject.

If not mentioned otherwise, the description and definition of the immunomodulatory substance(s) stated above is independently and mutatis mutandis applicable to the IL-2 and/or IL-2-like acting substance(s). Particularly, if not mentioned otherwise, the description and definition of the immunomodulatory substance(s) in respect of “directly” and “indirectly” affecting or capable of affecting the PBMCs is independently and mutatis mutandis applicable to the IL-2 and/or IL-2-like acting substance(s).

The IL-2 and/or IL-2-like acting substance(s) for use according to the present invention and/or the IL-2 and/or IL-2-like acting substance(s) of steps (B), (B₁) and/or (C) of the method, the pharmaceutical compositions, injectable dosage forms, topical dosage forms, medical devices and/or kits of parts, may be similar or identical, preferably identical, to any IL-2 and/or IL-2-like acting substance(s) naturally occurring in a vertebrate, preferably a human or a mammal, more preferably in the subject as defined in any of the embodiments according to the present invention. Preferably, the IL-2 and/or IL-2-like acting substance(s) may be similar or identical, preferably identical, to any peptide, protein or any fragment of these of IL-2 and/or IL-2-like acting substance(s) naturally occurring in a vertebrate, preferably a human or a mammal as defined above, more preferably in the subject as defined in any of the embodiments according to the present invention.

The IL-2 and/or IL-2-like acting substance(s) may comprise, preferably consist of an amino acid sequence which has an amino acid sequence identity to any peptide, protein or any fragment of an IL-2 and/or IL-2-like acting substance(s) naturally occurring in a vertebrate, preferably a mammal, more preferably a human or a mammal animal as defined above, more preferably in the subject as defined in any of the embodiments according to the present invention. More preferably, the amino acid sequence identity is with reference to amino acid sequence SEQ ID NO: 2 or any fragment thereof. Preferably, the amino acid sequence identity is 70% or more, even more preferably 85% or more, still more preferably 90% or more, still even more preferably 95% or more, further preferably 97% or more, even further preferably 98% or more, still further preferably 99% or more and 100% or less. Preferably, the amino acid sequence identity includes substitutions, insertions and/or deletions of amino acid residues in respect to the naturally occurring peptide, protein or any fragment of these.

Preferably, the total number of substitutions, insertions and/or deletions or the total number combined of substitutions, insertions and/or deletions of amino acid residues is 60 or less, more preferably 40 or less, even more preferably 20 or less, still more preferably 12 or less, still even more preferably 8 or less, further preferably 4 or less, even further preferably 2 or less and zero or more, still further preferably zero, preferably with reference to amino acid sequence SEQ ID NO: 2, wherein more preferably the subject is a human.

The IL-2 and/or IL-2-like acting substance(s) is identical or the amino acid sequence identity refers to an endogenous immunomodulatory substance(s) of the same genus and/or species the subject belongs to. The expression “is identical” typically means in this particular context that the IL-2 and/or IL-2-like acting substance(s) is in respect to its primary, secondary, tertiary and quaternary protein structure identical to an/the IL-2 and/or IL-2-like acting substance(s) of the same genus and/or species the subject belongs to, more preferably, also in view of the modification pattern, particularly glycosylations.

Preferably, the IL-2 and/or IL-2-like acting substance(s) for use according to the present invention and/or the IL-2 and/or IL-2-like acting substance(s) of steps (B), (B₁) and/or (C) of the method, the pharmaceutical compositions, injectable dosage forms, topical dosage forms, medical devices and/or kits of parts, may be derived from an individual belonging to the same genus and/or species as the subject, or which is identical to an/the IL-2 and/or IL-2-like acting substance(s) of the same genus and/or species the subject belongs to. For instance, in case the subject is: a human the IL-2 and/or IL-2-like acting substance(s) is human IL-2 and/or IL-2-like acting substance(s), more preferably human IL-2 and/or IL-2-like acting substance(s) comprising, even more preferably consisting of, preferably as effective amino acid sequence, the amino acid sequence SEQ ID NO: 2; a cow the IL-2 and/or IL-2-like acting substance(s) is bovine IL-2 and/or IL-2-like acting substance(s); a horse the IL-2 and/or IL-2-like acting substance(s) is equine IL-2 and/or IL-2-like acting substance(s); a donkey the IL-2 and/or IL-2-like acting substance(s) is donkey IL-2 and/or IL-2-like acting substance(s); an elephant the IL-2 and/or IL-2-like acting substance(s) is elephant IL-2 and/or IL-2-like acting substance(s); a sheep the IL-2 and/or IL-2-like acting substance(s) is sheep IL-2 and/or IL-2-like acting substance(s); a goat the IL-2 and/or IL-2-like acting substance(s) is goat IL-2 and/or IL-2-like acting substance(s); a pig the IL-2 and/or IL-2-like acting substance(s) is porcine IL-2 and/or IL-2-like acting substance(s); a rabbit the IL-2 and/or IL-2-like acting substance(s) is rabbit IL-2 and/or IL-2-like acting substance(s); a mouse the IL-2 and/or IL-2-like acting substance(s) is mouse IL-2 and/or IL-2-like acting substance(s); a rat the IL-2 and/or IL-2-like acting substance(s) is rat IL-2 and/or IL-2-like acting substance(s); a camel the IL-2 and/or IL-2-like acting substance(s) is camel IL-2 and/or IL-2-like acting substance(s); a dromedary the IL-2 and/or IL-2-like acting substance(s) is dromedary IL-2 and/or IL-2-like acting substance(s); a lama the IL-2 and/or IL-2-like acting substance(s) is lama IL-2 and/or IL-2-like acting substance(s); an alpaca the IL-2 and/or IL-2-like acting substance(s) is alpaca IL-2 and/or IL-2-like acting substance(s); a dog the IL-2 and/or IL-2-like acting substance(s) is dog IL-2 and/or IL-2-like acting substance(s); and/or a cat the IL-2 and/or IL-2-like acting substance(s) is cat IL-2 and/or IL-2-like acting substance(s).

Preferably, the IL-2 and/or IL-2-like acting substance(s) for use according to the present invention and/or the IL-2 and/or IL-2-like acting substance(s) of steps (B), (B₁) and/or (C) of the method, the pharmaceutical compositions, injectable dosage forms, topical dosage forms, medical devices and/or kits of parts, is not the subject's endogenous IL-2 and/or IL-2-like acting substance(s). In other words, preferably the IL-2 and/or IL-2-like acting substance(s) is not an IL-2 and/or IL-2-like acting substance(s) isolated from the subject's body, i.e. from the individual subject to be treated or prevented.

Preferably, the IL-2 and/or IL-2-like acting substance(s) for use according to the present invention and/or the IL-2 and/or IL-2-like acting substance(s) of steps (B), (B₁) and/or (C) of the method, the pharmaceutical compositions, injectable dosage forms, topical dosage forms, medical devices and/or kits of parts, may be, preferably, is a recombinant IL-2 and/or IL-2-like acting substance(s) which may be generated by any recombinant expression techniques known to the person skilled in the art, a chemically synthesized IL-2 and/or IL-2-like acting substance(s) synthesized by any production techniques known to the person skilled in the art, an artificially produced IL-2 and/or IL-2-like acting substance(s) produced by any production techniques known to the person skilled in the art, a naturally occurring IL-2 and/or IL-2-like acting substance(s) obtained from natural sources like an animal or human, or any combination thereof. More preferably, the IL-2 and/or IL-2-like acting substance(s) is a recombinant IL-2 and/or IL-2-like acting substance(s), chemically synthesized IL-2 and/or IL-2-like acting substance(s), artificially produced IL-2 and/or IL-2-like acting substance(s) or any combination thereof, even more preferably a recombinant IL-2 and/or IL-2-like acting substance(s), still even more preferably recombinant human IL-2, further preferably Aldesleukin, preferably produced in genetically modified Escherichia coli (produced by Novartis Pharmaceuticals UK, Limited and in Germany commercially available from Novartis Pharma GmbH under the tradename Proleukin®S).

Furthermore, the IL-2 and/or IL-2-like acting substance(s) for use according to the present invention and/or the IL-2 and/or IL-2-like acting substance(s) of steps (B), (B₁) and/or (C) of the method, the pharmaceutical compositions, injectable dosage forms, topical dosage forms, medical devices and/or kits of parts, may or may not be derivatized, stabilized, fused with other proteins or peptides, conjugated with polymers, contain amino acid analogue(s) or artificial amino acids, modified, covalently or non-covalently, for instance glycosylated or methylated by e.g. posttranslational modification and/or oligomerized, e.g. dimerized or trimerized as long as it is capable of affecting and/or affects LHs, peripheral T_{reg}s or PBMCs. More preferably, the modification pattern and/or oligomerization status is similar or identical to the peptide, protein or any fragment of these naturally occurring in a vertebrate, preferably a mammal, more preferably a human or a mammal animal as defined above, more preferably in the subject as defined in any of the embodiments according to the present invention.

Preferably, in any of the embodiments described herein it is intended to administer the IL-2 and/or IL-2-like acting substance(s) in an amount low enough to avoid a systemic increase of the IL-2 and/or IL-2-like acting substance(s)-concentration in the subject's body. Hence, the IL-2 and/or IL-2-like acting substance(s) may be administered in an amount that, when scaled to the entire body of the subject, the resulting systemic concentrations remain in the magnitude of the endogenous levels. Thereby, without wishing to be bound to theory, it is believed that the generation of an increased IL-2 and/or IL-2-like acting substance(s)-concentration for instance at sites of inflammation, like inflamed joint. As already stated above, for instance naïve T-cells can mature in the presence of antigen/autoantigen/allergen towards cytotoxic, which may act pro-inflammatory. Thereby decreasing, cancelling or even reversing the beneficial effects achieved by the present invention, particularly, the anti-inflammatory effect of the regulatory T-cells and/or helper T-cells, or of a subset thereof, which are presumably generated.

Preferably, in any of the embodiments described herein, particularly in step (B), (B₁) and/or (C), the IL-2 and/or IL-2-like acting substance(s) is administered in an amount that does not cause a systemic increase, more preferably an effective systemic increase, of the concentration of IL-2 and/or IL-2-like acting substance(s) in the subject, preferably the blood of the subject, and/or that causes a systemic increase of the concentration of the IL-2 and/or IL-2-like acting substance(s). Preferably, IL-2 and/or IL-2-like acting substance(s) is administered in an amount low enough that the systemic increase is non-effective.

Preferably, in any of the embodiments described herein, particularly in step (B), (B₁) and/or (C), the IL-2 and/or IL-2-like acting substance(s) is administered in an amount that causes a local increase, preferably an effective local increase, of the concentration of the IL-2 and/or IL-2-like acting substance(s) in the subject, preferably in the skin tissue of the subject. The skin tissue is preferred, because unlike administration into the blood, no or less flushing away and dilution of the IL-2 and/or IL-2-like acting substance(s) takes place.

Preferably, in any of the embodiments described herein, particularly in step (B), (B₁) and/or (C), the generated increased concentration, preferably the generated effective increased concentration of the IL-2 and/or IL-2-like acting substance(s) administered is only local but not systemic in the subject.

Preferably, in any of the embodiments described herein, particularly in step (B), (B₁) and/or (C), the IL-2 and/or IL-2-like acting substance(s) is administered in an amount that does not cause a systemic activation, preferably an effective systemic activation, of the respective receptor of the IL-2 and/or IL-2-like acting substance(s) of a cell, and/or that does not cause a systemic generation, preferably an effective systemic generation, of the IL-2 and/or IL-2-like acting substance(s), preferably in the subject.

Preferably, in any of the embodiments described herein, particularly in step (B), (B₁) and/or (C), the IL-2 and/or IL-2-like acting substance(s) is administered in an amount that causes a local activation, preferably an effective local activation of the respective receptor of the IL-2 and/or IL-2-like acting substance(s) of a cell, and/or causes a local generation, preferably an effective local generation of the IL-2 and/or IL-2-like acting substance(s), preferably in the subject. Preferably, the activation and/or generation, more preferably the effective generation and/or effective activation is only local but not systemic in the subject.

Generally, 1 ng IL-2 corresponds to 16.4 IU IL-2 or less. Some of the IL-2 might be denatured or otherwise defect in that the biological activity of IL-2 has been lost while the mass remains constant.

Preferably, in any of the embodiments described herein, particularly in step (B), (B₁) and/or (C), IL-2-like acting substance(s), preferably the IL-2, is administered in an effective amount, preferably an effective total amount.

Preferably, in any of the embodiments described herein, particularly in step (B), (B₁) and/or (C), IL-2-like acting IL-2 substance(s) is administered in an amount, preferably a total amount, equivalent to 20 IU IL-2/kg body mass of a subject or less, more preferably IU IL-2/kg body mass of a subject or less, preferably per administration dose, to a skin of the subject. Even more preferably, the IL-2-like acting IL-2 substance(s) is administered in an amount equivalent to 8 IU IL-2/kg or less, still more preferably 6 IU IL-2/kg, still even more preferably 5 IU IL-2/kg or less, further preferably 4 IU IL-2/kg or less. There is no lower limit for the amount, preferably the total amount, per kg body mass as long IL-2 as the beneficial effects can be achieved, however, usually, a lower limit is and amount equivalent to 0.5 IU IL-2/kg or more, more preferably 1 IU IL-2/kg or more, even more preferably 2 IU IL-2/kg or more. More preferably, the IL-2-like acting IL-2 substance(s) is administered in an amount, preferably a total amount, in the range of equivalent to IU IL-2/kg or less and 0.5 IU IL-2/kg or more, even more preferably 10 IU IL-2/kg or less and 0.5 IU IL-2/kg or more, still more preferably 8 IU IL-2/kg or less and 0.5 IU IL-2/kg or more, still even more preferably 6 IU IL-2/kg or less and 1 IU IL-2/kg or more, further preferably 5 IU IL-2/kg or less and 1 IU IL-2/kg or more, even further preferably 4 IU IL-2/kg or less and 1 IU IL-2/kg or more.

Preferably, in any of the embodiments described herein, particularly in step (B), (B₁) and/or (C), IL-2-like acting IL-2 substance(s) is administered in an amount, preferably a total amount, equivalent in activity to 1.2 ng IL-2/kg body mass of a subject or less, more preferably 0.6 ng IL-2/kg body mass of a subject or less, preferably per administration dose, to a skin of the subject. Even more preferably, IL-2, preferably IL-2-like acting IL-2 substance(s) is administered in an amount equivalent in activity to 0.5 ng IL-2/kg or less, still more preferably 0.37 ng IL-2/kg, still even more preferably 0.3 ng IL-2/kg or less, further preferably 0.25 ng IL-2/kg or less. There is no lower limit for the amount, preferably the total amount, per kg body mass as long IL-2 as the beneficial effects can be achieved, however, usually, a lower limit is an amount equivalent in activity to 0.03 ng IL-2/kg or more, more preferably 0.06 ng IL-2/kg or more, even more preferably 0.12 ng IL-2/kg or more. More preferably, IL-2-like acting IL-2 substance(s) is administered in an amount, preferably a total amount, in the range of equivalent in activity to 1.2 ng IL-2/kg or less and 0.03 ng IL-2/kg or more, even more preferably 0.6 ng IL-2/kg or less and 0.03 ng IL-2/kg or more, still more preferably 0.5 ng IL-2/kg or less and 0.03 ng IL-2/kg or more, still even more preferably 0.37 ng IL-2/kg or less and 0.06 ng IL-2/kg or more, further preferably 0.3 ng IL-2/kg or less and 0.06 ng IL-2/kg or more, even further preferably 0.25 ng IL-2/kg or less and 0.12 ng IL-2/kg or more.

Preferably, in any of the embodiments described herein, particularly in step (B), (B₁) and/or (C), the IL-2-like acting IL-2 substance(s) is administered in an amount, preferably a total amount, equivalent to 1,000 IU IL-2 or less, more preferably 500 IU IL-2 or less, even more preferably 400 IU IL-2 or less, still more preferably 300 IU IL-2 or less, still even more preferably 250 IU IL-2 or less. There is no lower limit for the amount, preferably the total amount, as long IL-2 as the beneficial effects can be achieved, however, usually, a lower limit is an amount equivalent to 25 IU IL-2 or more, more preferably 50 IU IL-2 or more, even more preferably 100 IU IL-2 or more. More preferably, the IL-2-like acting IL-2 substance(s) is administered in an amount, preferably a total amount, in the rang IL-2e of equivalent to 1,000 IU IL-2 or less and 25 IU IL-2 or more, even more preferably 500 IU IL-2 or less and 25 IU IL-2 or more, still more preferably 400 IU IL-2 or less and 50 IU IL-2 or more, still even more preferably 300 IU IL-2 or less and 50 IU IL-2 or more, further preferably 250 IU IL-2 or less and 100 IU IL-2 or more.

Preferably, in any of the embodiments described herein, particularly in step (B), (B₁) and/or (C), IL-2-like acting IL-2 substance(s) is administered in an amount, preferably a total amount, equivalent in activity to 61 ng IL-2 or less, more preferably 30.5 ng IL-2 or less, even more preferably 24.5 ng IL-2 or less, still more preferably 18.3 ng IL-2 or less, still even more preferably 12.2 ng IL-2 or less. There is no lower limit for the amount, preferably the total amount, as long IL-2 as the beneficial effects can be achieved, however, usually, a lower limit is and amount equivalent in activity to 2 ng IL-2 or more, more preferably 3 ng IL-2 or more, even more preferably 6.1 ng IL-2 or more. More preferably, the IL-2-like acting IL-2 substance(s) is administered in an amount, preferably the total amount, in the range of equivalent in activity to 61 ng IL-2 or less and 2 ng IL-2 or more, even more preferably 30.5 ng IL-2 or less and 2 ng IL-2 or more, still more preferably 24.5 ng IL-2 or less and 3 ng IL-2 or more, still even more preferably 28.3 ng IL-2 or less and 3 ng IL-2 or more, still even more preferably 12.1 ng IL-2 or less and 6.1 ng IL-2 or more.

Preferably, in any of the embodiments described herein, particularly in step (B), (B₁) and/or (C), IL-2 is administered in an amount, preferably a total amount, of 20 IU/kg body mass of a subject or less, more preferably 10 IU/kg body mass of a subject or less, preferably per administration dose, to a skin of the subject. Even more preferably, IL-2 is administered in an amount of 8 IU/kg or less, still more preferably 6 IU/kg, still even more preferably 5 IU/kg or less, further preferably 4 IU/kg or less. There is no lower limit for the amount, preferably the total amount, per kg body mass as long as the beneficial effects can be achieved, however, usually, a lower limit is 0.5 IU/kg or more, more preferably 1 IU/kg or more, even more preferably 2 IU/kg or more. More preferably, IL-2 is administered in an amount, preferably a total amount, in the range of 20 IU/kg or less and 0.5 IU/kg or more, even more preferably IU/kg or less and 0.5 IU/kg or more, still more preferably 8 IU/kg or less and 0.5 IU/kg or more, still even more preferably 6 IU/kg or less and 1 IU/kg or more, further preferably 5 IU/kg or less and 1 IU/kg or more, even further preferably 4 IU/kg or less and 1 IU/kg or more.

Preferably, in any of the embodiments described herein, particularly in step (B), (B₁) and/or (C), IL-2 is administered in an amount, preferably a total amount, of 1.2 ng/kg body mass of a subject or less, more preferably 0.6 ng/kg body mass of a subject or less, preferably per administration dose, to a skin of the subject. Even more preferably, IL-2 is administered in an amount of 0.5 ng/kg or less, still more preferably 0.37 ng/kg, still even more preferably 0.3 ng/kg or less, further preferably 0.25 ng/kg or less. There is no lower limit for the amount, preferably the total amount, per kg body mass as long as the beneficial effects can be achieved, however, usually, a lower limit is 0.03 ng/kg or more, more preferably 0.06 ng/kg or more, even more preferably 0.12 ng/kg or more. More preferably, IL-2 is administered in an amount, preferably a total amount, in the range of 1.2 ng/kg or less and 0.03 ng/kg or more, even more preferably 0.6 ng/kg or less and 0.03 ng/kg or more, still more preferably 0.5 ng/kg or less and 0.03 ng/kg or more, still even more preferably 0.37 ng/kg or less and 0.06 ng/kg or more, further preferably 0.3 ng/kg or less and 0.06 ng/kg or more, even further preferably 0.25 ng/kg or less and 0.12 ng/kg or more.

Preferably, in any of the embodiments described herein, particularly in step (B), (B₁) and/or (C), the IL-2 is administered in an amount, preferably a total amount, of 1,000 IU or less, more preferably 500 IU or less, even more preferably 400 IU or less, still more preferably 300 IU or less, still even more preferably 250 IU or less. There is no lower limit for the amount, preferably the total amount, as long as the beneficial effects can be achieved, however, usually, a lower limit is 25 IU or more, more preferably 50 IU or more, even more preferably 100 IU or more. More preferably, IL-2 is administered in an amount, preferably a total amount, in the range of 1,000 IU or less and 25 IU or more, even more preferably 500 IU or less and 25 IU or more, still more preferably 400 IU or less and 50 IU or more, still even more preferably 300 IU or less and 50 IU or more, further preferably 250 IU or less and 100 IU or more.

Preferably, in any of the embodiments described herein, particularly in step (B), (B₁) and/or (C), the IL-2 is administered in an amount, preferably a total amount, of 61 ng or less, more preferably 30.5 ng or less, even more preferably 24.5 ng or less, still more preferably 18.3 ng or less, still even more preferably 12.2 ng or less. There is no lower limit for the amount, preferably the total amount, as long as the beneficial effects can be achieved, however, usually, a lower limit is 2 ng or more, more preferably 3 ng or more, even more preferably 6.1 ng or more. More preferably, IL-2 is administered in an amount, preferably the total amount, in the range of 61 ng or less and 2 ng or more, even more preferably 30.5 ng or less and 2 ng or more, still more preferably 24.5 ng or less and 3 ng or more, still even more preferably 28.3 ng or less and 3 ng or more, still even more preferably 12.1 ng or less and 6.1 ng or more.

Without wishing to be bound to theory, it is believed that IL-2-like acting substances like IL-2 proliferate regulatory T-cells and helper T-cells, or a subset thereof, thereby resulting in enhancing and maintaining the effect over a longer period of time. As can be seen from the examples, IL-2 has amongst others have the effect of synergistically prolonging, enhancing and/or boosting the beneficial effect of the first immunomodulatory substance(s), i.e. immunomodulatory substance(s) other than IL-2, like for instance IFN-γ, IL-4 or BDNF.

The expression “IL-4-like acting substance” or “IL-4-like acting substance(s)” as mentioned in any of the embodiments described herein can be, preferably is, any substance that exerts a similar or identical effect to a IL-4 in the body of the subject when administered thereto. The IL-4-like acting substance(s) may encompass precursors, prodrugs and propeptides of such substances which are usually not active as such, i.e. have no immunomodulatory activity, but which, upon use in accordance with the present invention, are converted into the actually active IL-4-like acting substance(s). Hence, it may be any substance, molecule, peptide, protein, protein-analogue, protein-variant, derivative, fragment, biopharmaceutical, inductor, precursor, prodrug, mutein, co-drug and/or any derivative, fragment and/or pharmaceutically acceptable salt of any of these. Therefore IL-4-like acting substance(s) may:
activates or is/are capable of activating a respective IL-4-receptor(s) of a cell. The IL-4-receptor(s) may for instance be the receptor of the LHs, peripheral T_{reg}s or PBMCs; and/or
results in or causes or is capable of resulting in or causing the generation of a IL-4 and/or a IL-4-like acting substance(s).

Preferably, the IL-4-like acting substance(s) activates or is capable of activating the respective IL-4-receptor(s). The activating or the capability of activating the respective IL-4-receptor(s) may be directly and/or it may be indirectly.

Preferably, the IL-4-like acting substance(s) activates or is/are capable of activating a respective IL-4-receptor(s) within the skin of the subject. The IL-4-like acting substance(s) may be any naturally occurring or artificial IL-4-like acting substance(s) as long as it has a sufficient biologic activity, i.e. an effective cross-reactivity in the subject, particularly is capable of activating the respective IL-4-receptor(s). Preferably, the IL-4-like acting substance(s) is known to the person skilled in the art at the effective date of filing of the present invention.

Preferably, the IL-4-like acting substance is any peptide, protein, protein analogue, protein-variant or any derivative, fragment, biopharmaceutical, inductor, precursor, prodrug, mutein, co-drug and/or pharmaceutically acceptable salt of any of these.

In a preferred embodiment, the IL-4-like acting substance(s) may be as detailed below amongst others a IL-4 and/or any derivative, fragment, protein-analogue, peptide, protein-variant, biopharmaceutical, inductor, precursor, prodrug, mutein, co-drug, propeptide thereof and/or any pharmaceutically acceptable salt of any of these.

More preferably, the IL-4-like acting substance(s) is an IL-4 and/or any derivative, fragment, protein-analogue, peptide, protein-variant, biopharmaceutical, inductor, precursor, prodrug, mutein, co-drug, propeptide thereof and/or any pharmaceutically acceptable salt of any of these.

The expression “IL-4” as mentioned in any of the embodiments described herein preferably has to be interpreted broad. Typically, the IL-4 activates or is are capable of activating a respective IL-4-receptor(s) of a cell. The IL-4-receptor(s) may for instance be the receptor of the LHs, peripheral T_{reg}s or PBMCs. Preferably, the activating or capability of activating is within the skin of the subject, more preferably locally in effective amounts within the skin of the subject, even more preferably not in amounts systemically effective within the subject's body. Preferably the IL-4 may be any type of IL-4 known to the person skilled in the art. Furthermore, the present invention encompass the use or medical use of precursors, prodrugs and propeptides of such IL-4 which are usually not active as such, i.e. have no immunomodulatory activity, but which, upon use in accordance with the present invention, are converted into the actually active IL-4. Hence, these may be used in the method or the medical use as described herein or in preparation of the pharmaceutical compositions, injectable dosage form, topical dosage forms, medical devices and/or kit of parts as described herein.

Preferably, the IL-4 may be any peptide, protein, protein-analogue, protein-variant or any derivative, fragment, biopharmaceutical, inductor, precursor, prodrug, mutein, co-drug and/or pharmaceutically acceptable salt of any of these.

The IL-4 does not necessarily be derived from or be identical to the IL-4 of the same genus and/or species the subject belongs to, it may be any naturally occurring or an artificial IL-4, as long as it has a sufficient biologic activity, i.e. an effective cross-reactivity in the subject, particularly is capable of activating the respective IL-4-receptor(s). Preferably, the biologic activity of the IL-4 and/or IL-4-like acting substance(s) is sufficient to achieve the treating and/or preventing of the inflammatory disease, immunological disease and/or autoimmunological disease, preferably in the subject.

An IL-4 and/or an IL-4-like acting substance typically affects cells by binding and activating the respective IL-4-receptor on the cells. Hence, the activating of the respective receptor typically leads to the affectation of the cell as described above for the immunomodulatory substance. Hence, any embodiment mentioned herein for the immunomodulatory substance relating to the affecting cells like LHs, peripheral T_{reg}s or PBMCs is independently and mutatis mutandis applicable to the IL-4 and the IL-4-like acting substance.

The activating or the capability of activating the respective IL-4-receptor(s) by the IL-4 and/or IL-4-like acting substance(s) may be directly and/or it may be indirectly.

If not mentioned otherwise, the description and definition of the cytokine(s) or the cytokine-like acting substance stated above is independently and mutatis mutandis applicable to the IL-4 and/or IL-4-like acting substance(s). Particularly, if not mentioned otherwise, the description and definition of the cytokine(s) or the cytokine-like acting substance in respect of “directly” and “indirectly” activating or capable of activating the respective IL-4-receptor(s), is independently and mutatis mutandis applicable to the IL-4 and/or IL-4-like acting substance(s).

Preferably, the IL-4 and/or IL-4-like acting substance(s) for use according to the present invention and/or the IL-4 and/or IL-4-like acting substance(s) of steps (B), (B₁) and/or (C) of the method, the pharmaceutical compositions, injectable dosage forms, topical dosage forms, medical devices and/or kits of parts, is capable of affecting and/or affects the LHs, peripheral T_{reg}s or PBMCs. Preferably, the PBMCs are naïve PBMCs, more preferably lymphocytes, even more preferably T-cells and/or B-cells, still more preferably naïve lymphocytes, still even more preferably naïve B-cells and/or naïve T-cells, further preferably naïve T-cells.

More preferably, the IL-4 and/or IL-4-like acting substance(s) is capable of directly and/or indirectly affecting and/or affects the LHs, peripheral T_{reg}s or PBMCs.

More preferably, the IL-4 and/or IL-4-like acting substance(s) is capable of directly and/or indirectly affecting and/or affects the LHs, peripheral T_{reg}s or PBMCs in the subject as defined in any of the embodiments according to the present invention, further preferably within the skin of the subject.

If not mentioned otherwise, the description and definition of the immunomodulatory substance(s) stated above is independently and mutatis mutandis applicable to the IL-4 and/or IL-4-like acting substance(s). Particularly, if not mentioned otherwise, the description and definition of the immunomodulatory substance(s) in respect of “directly” and “indirectly” affecting or capable of affecting the PBMCs is independently and mutatis mutandis applicable to the IL-4 and/or IL-4-like acting substance(s).

The IL-4 and/or IL-4-like acting substance(s) for use according to the present invention and/or the IL-4 and/or IL-4-like acting substance(s) of steps (B), (B₁) and/or (C) of the method, the pharmaceutical compositions, injectable dosage forms, topical dosage forms, medical devices and/or kits of parts, may be similar or identical, preferably identical, to any IL-4 and/or IL-4-like acting substance(s) naturally occurring in a vertebrate, preferably a human or a mammal, more preferably in the subject as defined in any of the embodiments according to the present invention. Preferably, the IL-4 and/or IL-4-like acting substance(s) may be similar or identical, preferably identical, to any peptide, protein or any fragment of these of IL-4 and/or IL-4-like acting substance(s) naturally occurring in a vertebrate, preferably a human or a mammal as defined above, more preferably in the subject as defined in any of the embodiments according to the present invention.

The IL-4 and/or IL-4-like acting substance(s) may comprise, preferably consist of an amino acid sequence which has an amino acid sequence identity to any peptide, protein or any fragment of an IL-4 and/or IL-4-like acting substance(s) naturally occurring in a vertebrate, preferably a mammal, more preferably a human or a mammal animal as defined above, more preferably in the subject as defined in any of the embodiments according to the present invention More preferably, the amino acid sequence identity is with reference to amino acid sequence SEQ ID NO: 3 or any fragment thereof. Preferably, the amino acid sequence identity is 70% or more, even more preferably 85% or more, still more preferably 90% or more, still even more preferably 95% or more, further preferably 97% or more, even further preferably 98% or more, still further preferably 99% or more and 100% or less. Preferably, the amino acid sequence identity includes substitutions, insertions and/or deletions of amino acid residues in respect to the naturally occurring peptide, protein or any fragment of these.

Preferably, the total number of substitutions, insertions and/or deletions or the total number combined of substitutions, insertions and/or deletions of amino acid residues is 60 or less, more preferably 40 or less, even more preferably 20 or less, still more preferably 12 or less, still even more preferably 8 or less, further preferably 4 or less, even further preferably 2 or less and zero or more, still further preferably zero, preferably with reference to amino acid sequence SEQ ID NO: 3, wherein more preferably the subject is a human.

The IL-4 and/or IL-4-like acting substance(s) may be identical to an endogenous immunomodulatory substance(s) of the same genus and/or species the subject belongs to or the amino acid sequence identity refers to an endogenous immunomodulatory substance(s) of the same genus and/or species the subject belongs to. The expression “is identical” typically means in this particular context that the IL-4 and/or IL-4-like acting substance(s) is in respect to its primary, secondary, tertiary and quaternary protein structure identical to an/the IL-4 and/or IL-4-like acting substance(s) of the same genus and/or species the subject belongs to, more preferably, also in view of the modification pattern, particularly glycosylations.

The IL-4 and/or IL-4-like acting substance(s) for use according to the present invention and/or the IL-4 and/or IL-4-like acting substance(s) of steps (B), (B₁) and/or (C) of the method, the pharmaceutical compositions, injectable dosage forms, topical dosage forms, medical devices and/or kits of parts, may be derived from an individual belonging to the same genus and/or species as the subject, or which is identical to an/the IL-4 and/or IL-4-like acting substance(s) of the same genus and/or species the subject belongs to. For instance, in case the subject is: a human the IL-4 and/or IL-4-like acting substance(s) is human IL-4 and/or IL-4-like acting substance(s), more preferably human IL-4 and/or IL-4-like acting substance(s) comprising, even more preferably consisting of, preferably as effective amino acid sequence, the amino acid sequence SEQ ID NO: 3; a cow the IL-4 and/or IL-4-like acting substance(s) is bovine IL-4 and/or IL-4-like acting substance(s); a horse the IL-4 and/or IL-4-like acting substance(s) is equine IL-4 and/or IL-4-like acting substance(s); a donkey the IL-4 and/or IL-4-like acting substance(s) is donkey IL-4 and/or IL-4-like acting substance(s); an elephant the IL-4 and/or IL-4-like acting substance(s) is elephant IL-4 and/or IL-4-like acting substance(s); a sheep the IL-4 and/or IL-4-like acting substance(s) is sheep IL-4 and/or IL-4-like acting substance(s); a goat the IL-4 and/or IL-4-like acting substance(s) is goat IL-4 and/or IL-4-like acting substance(s); a pig the IL-4 and/or IL-4-like acting substance(s) is porcine IL-4 and/or IL-4-like acting substance(s); a rabbit the IL-4 and/or IL-4-like acting substance(s) is rabbit IL-4 and/or IL-4-like acting substance(s); a mouse the IL-4 and/or IL-4-like acting substance(s) is mouse IL-4 and/or IL-4-like acting substance(s); a rat the IL-4 and/or IL-4-like acting substance(s) is rat IL-4 and/or IL-4-like acting substance(s); a camel the IL-4 and/or IL-4-like acting substance(s) is camel IL-4 and/or IL-4-like acting substance(s); a dromedary the IL-4 and/or IL-4-like acting substance(s) is dromedary IL-4 and/or IL-4-like acting substance(s); a lama the IL-4 and/or IL-4-like acting substance(s) is lama IL-4 and/or IL-4-like acting substance(s); an alpaca the IL-4 and/or IL-4-like acting substance(s) is alpaca IL-4 and/or IL-4-like acting substance(s); a dog the IL-4 and/or IL-4-like acting substance(s) is dog IL-4 and/or IL-4-like acting substance(s); and/or a cat the IL-4 and/or IL-4-like acting substance(s) is cat IL-4 and/or IL-4-like acting substance(s).

Preferably, the IL-4 and/or IL-4-like acting substance(s) for use according to the present invention and/or the IL-4 and/or IL-4-like acting substance(s) of steps (B), (B₁) and/or (C) of the method, the pharmaceutical compositions, injectable dosage forms, topical dosage forms, medical devices and/or kits of parts, is not the subject's endogenous IL-4 and/or IL-4-like acting substance(s). In other words, preferably the IL-4 and/or IL-4-like acting substance(s) is not an IL-4 and/or IL-4-like acting substance(s) isolated from the subject's body, i.e. from the individual subject to be treated or prevented.

Preferably, the IL-4 and/or IL-4-like acting substance(s) for use according to the present invention and/or the IL-4 and/or IL-4-like acting substance(s) of steps (B), (B₁) and/or (C) of the method, the pharmaceutical compositions, injectable dosage forms, topical dosage forms, medical devices and/or kits of parts, may be, preferably, is a recombinant IL-4 and/or IL-4-like acting substance(s) which may be generated by any recombinant expression techniques known to the person skilled in the art, a chemically synthesized IL-4 and/or IL-4-like acting substance(s) synthesized by any production techniques known to the person skilled in the art, an artificially produced IL-4 and/or IL-4-like acting substance(s) produced by any production techniques known to the person skilled in the art, a naturally occurring IL-4 and/or IL-4-like acting substance(s) obtained from natural sources like an animal or human, or any combination thereof. More preferably, the IL-4 and/or IL-4-like acting substance(s) is a recombinant IL-4 and/or IL-4-like acting substance(s), chemically synthesized IL-4 and/or IL-4-like acting substance(s), artificially produced IL-4 and/or IL-4-like acting substance(s) or any combination thereof, even more preferably a recombinant IL-4 and/or IL-4-like acting substance(s), still even more preferably recombinant human IL-4, preferably produced in genetically modified Escherichia coli (produced and commercially available from R&D Systems under the name “Recombinant Human IL-4 Protein”, catalogue number 204-IL/CF [carrier free]).

Furthermore, the IL-4 and/or IL-4-like acting substance(s) for use according to the present invention and/or the IL-4 and/or IL-4-like acting substance(s) of steps (B), (B₁) and/or (C) of the method, the pharmaceutical compositions, injectable dosage forms, topical dosage forms, medical devices and/or kits of parts, may or may not be derivatized, stabilized, fused with other proteins or peptides, conjugated with polymers, contain amino acid analogue(s) or artificial amino acids, modified, covalently or non-covalently, for instance glycosylated or methylated by e.g. posttranslational modification and/or oligomerized, e.g. dimerized or trimerized as long as it is capable of affecting and/or affects LHs, peripheral T_{reg}s or PBMCs. More preferably, the modification pattern and/or oligomerization status is similar or identical to the peptide, protein or any fragment of these naturally occurring in a vertebrate, preferably a mammal, more preferably a human or a mammal animal as defined above, more preferably in the subject as defined in any of the embodiments according to the present invention.

Preferably, in any of the embodiments described herein it is intended to administer the IL-4 and/or IL-4-like acting substance(s) in an amount low enough to avoid a systemic increase of the IL-4 and/or IL-4-like acting substance(s)-concentration in the subject's body. Hence, the IL-4 and/or IL-4-like acting substance(s) may be administered in an amount that, when scaled to the entire body of the subject, the resulting systemic concentrations remain in the magnitude of the endogenous levels. Thereby, without wishing to be bound to theory, it is believed that the generation of an increased IL-4 and/or IL-4-like acting substance(s)-concentration for instance at sites of inflammation, like inflamed joint. As already stated above, for instance naïve T-cells can mature in the presence of antigen/autoantigen/allergen towards cytotoxic, which may act pro-inflammatory. Thereby decreasing, cancelling or even reversing the beneficial effects achieved by the present invention, particularly, the anti-inflammatory effect of the regulatory T-cells and/or helper T-cells, or of a subset thereof, which are presumably generated.

Preferably, in any of the embodiments described herein, particularly in step (B), (B₁) and/or (C), the IL-4 and/or IL-4-like acting substance(s) is administered in an amount that does not cause a systemic increase, more preferably an effective systemic increase, of the concentration of IL-4 and/or IL-4-like acting substance(s) in the subject, preferably the blood of the subject, and/or that causes a systemic increase of the concentration of the IL-4 and/or IL-4-like acting substance(s). Preferably, IL-4 and/or IL-4-like acting substance(s) is administered in an amount low enough that the systemic increase is non-effective.

Preferably, in any of the embodiments described herein, particularly in step (B), (B₁) and/or (C), the IL-4 and/or IL-4-like acting substance(s) is administered in an amount that causes a local increase, preferably an effective local increase, of the concentration of the IL-4 and/or IL-4-like acting substance(s) in the subject, preferably in the skin tissue of the subject. The skin tissue is preferred, because unlike administration into the blood, no or less flushing away and dilution of the IL-4 and/or IL-4-like acting substance(s) takes place.

Preferably, in any of the embodiments described herein, particularly in step (B), (B₁) and/or (C), the generated increased concentration, preferably the generated effective increased concentration of the IL-4 and/or IL-4-like acting substance(s) administered is only local but not systemic in the subject.

Preferably, in any of the embodiments described herein, particularly in step (B), (B₁) and/or (C), the IL-4 and/or IL-4-like acting substance(s) is administered in an amount that does not cause a systemic activation, preferably an effective systemic activation, of the respective receptor of the IL-4 and/or IL-4-like acting substance(s) of a cell, and/or that does not cause a systemic generation, preferably an effective systemic generation, of the IL-4 and/or IL-4-like acting substance(s), preferably in the subject.

Preferably, in any of the embodiments described herein, particularly in step (B), (B₁) and/or (C), the IL-4 and/or IL-4-like acting substance(s) is administered in an amount that causes a local activation, preferably an effective local activation of the respective receptor of the IL-4 and/or IL-4-like acting substance(s) of a cell, and/or causes a local generation, preferably an effective local generation of the IL-4 and/or IL-4-like acting substance(s), preferably in the subject. Preferably, the activation and/or generation, more preferably the effective generation and/or effective activation is only local but not systemic in the subject.

Preferably, in any of the embodiments described herein, particularly in step (B), (B₁) and/or (C), the IL-4-like acting substance(s), preferably the IL-4, is administered in an effective amount, preferably an effective total amount.

Preferably, in any of the embodiments described herein, particularly in step (B), (B₁) and/or (C), the IL-4-like acting substance(s) is administered in an amount, preferably a total amount, equivalent in activity to 20 ng IL-4/kg body mass of the subject or less, preferably per administration dose, to the skin of the subject. More preferably, IL-4 is administered in an amount equivalent in activity to ng IL-4/kg or less, even more preferably 5 ng IL-4/kg or less, still more preferably 2.5 ng IL-4/kg or less, still even more preferably 1.5 ng IL-4/kg or less, further preferably 1 ng IL-4/kg or less, still even more preferably 0.4 ng IL-4/kg or less, even further preferably 0.2 ng IL-4/kg or less. There is no lower limit for the amount, preferably the total amount, per kg body mass as long as the beneficial effects can be achieved, however, usually, a lower limit is an amount equivalent in activity to 0.002 ng IL-4/kg or more, more preferably 0.025 ng IL-4/kg or more, even more preferably 0.01 ng IL-4/kg or more, still more preferably 0.02 mg/kg or more, still even more preferably 0.05 ng IL-4/kg or more, further preferably 0.075 ng IL-4/kg or more. More preferably, the IL-4-like acting substance(s) is administered in an amount, preferably a total amount, in the range of equivalent in activity to 20 ng IL-4/kg or less and 0.002 ng IL-4/kg or more, even more preferably 10 ng IL-4/kg or less and 0.025 ng IL-4/kg or more, still more preferably 5 ng IL-4/kg or less and 0.01 ng IL-4/kg or more, still even more preferably 2.5 ng IL-4/kg or less and 0.01 ng IL-4/kg or more, further preferably 1.5 ng IL-4/kg or less and 0.02 ng IL-4/kg or more, even further preferably 1 ng IL-4/kg or less and 0.02 ng IL-4/kg or more, still further preferably 0.4 ng IL-4/kg or less and 0.05 ng IL-4/kg or more and still even further preferably 0.2 ng IL-4/kg or less and 0.075 ng IL-4/kg or more.

Preferably, in any of the embodiments described herein, particularly in step (B), (B₁) and/or (C), the IL-4-like acting substance(s) is administered, preferably per administration dose, in an amount, preferably a total amount, equivalent in activity to 1,000 ng IL-4 or less, more preferably 500 ng IL-4 or less, even more preferably 80 ng IL-4 or less, still more preferably 50 ng IL-4 or less, still even more preferably 20 ng IL-4 or less, still even more preferably 15 ng IL-4 or less. There is no lower limit for the amount, preferably the total amount, as long as the beneficial effects can be achieved, however, usually, a lower limit is an amount equivalent in activity to 0.1 ng IL-4 or more, more preferably 0.5 ng IL-4 or more, even more preferably 1 ng IL-4 or more and still even more preferably 2 ng IL-4 or more. More preferably, the IL-4-like acting substance(s), is administered, preferably per administration dose, in an amount, preferably a total amount, in the range of equivalent in activity to 1,000 ng IL-4 or less and 0.1 ng IL-4 or more, even more preferably 500 ng IL-4 or less and 0.1 ng IL-4 or more, still more preferably 80 ng IL-4 or less and 0.5 ng IL-4 or more, still even more preferably 50 ng IL-4 or less and 0.5 ng IL-4 or more, further preferably 20 ng IL-4 or less and 1 ng IL-4 or more, even further preferably ng IL-4 or less and 2 ng IL-4 or more.

Preferably, in any of the embodiments described herein, particularly in step (B), (B₁) and/or (C), IL-4 is administered in an amount, preferably a total amount, of 20 ng/kg body mass of the subject or less, preferably per administration dose, to the skin of the subject. More preferably, IL-4 is administered in an amount of 10 ng/kg or less, even more preferably 5 ng/kg or less, still more preferably 2.5 ng/kg or less, still even more preferably 1.5 ng/kg or less, further preferably 1 ng/kg or less, still even more preferably 0.4 ng/kg or less, even further preferably 0.2 ng/kg or less. There is no lower limit for the amount, preferably the total amount, per kg body mass as long as the beneficial effects can be achieved, however, usually, a lower limit is 0.002 ng/kg or more, more preferably 0.025 ng/kg or more, even more preferably 0.01 ng/kg or more, still more preferably 0.02 mg/kg or more, still even more preferably 0.05 ng/kg or more, further preferably 0.075 ng/kg or more. More preferably, IL-4 is administered in an amount, preferably a total amount, in the range of 20 ng/kg or less and 0.002 ng/kg or more, even more preferably 10 ng/kg or less and 0.025 ng/kg or more, still more preferably ng/kg or less and 0.01 ng/kg or more, still even more preferably 2.5 ng/kg or less and 0.01 ng/kg or more, further preferably 1.5 ng/kg or less and 0.02 ng/kg or more, even further preferably 1 ng/kg or less and 0.02 ng/kg or more, still further preferably 0.4 ng/kg or less and 0.05 ng/kg or more and still even further preferably 0.2 ng/kg or less and 0.075 ng/kg or more.

Preferably, in any of the embodiments described herein, particularly in step (B), (B₁) and/or (C), the IL-4 is administered, preferably per administration dose, in an amount, preferably a total amount, of 1,000 ng or less, more preferably 500 ng or less, even more preferably 80 ng or less, still more preferably 50 ng or less, still even more preferably 20 ng or less, still even more preferably 15 ng or less. There is no lower limit for the amount, preferably the total amount, as long as the beneficial effects can be achieved, however, usually, a lower limit is 0.1 ng or more, more preferably 0.5 ng or more, even more preferably 1 ng or more and still even more preferably 2 ng or more. More preferably, IL-4 is administered, preferably per administration dose, in an amount, preferably a total amount, in the range of 1,000 ng or less and 0.1 ng or more, even more preferably 500 ng or less and 0.1 ng or more, still more preferably 80 ng or less and 0.5 ng or more, still even more preferably 50 ng or less and 0.5 ng or more, further preferably 20 ng or less and 1 ng or more, even further preferably 15 ng or less and 2 ng or more.

As can be seen from the examples, IL-4 has amongst others the beneficial effects of avoiding relapses in case of multiple sclerosis and conveying condition stabilization or even improvement and reduction of fatigue as indicated by a decreasing SHILD-score.

The expression “BDNF-like acting substance” or “BDNF-like acting substance(s)” as mentioned in any of the embodiments described herein can be, preferably is, any substance that exerts a similar or identical effect to a BDNF in the body of the subject when administered thereto. The BDNF-like acting substance(s) may encompass precursors, prodrugs and propeptides of such substances which are usually not active as such, i.e. have no immunomodulatory activity, but which, upon use in accordance with the present invention, are converted into the actually active BDNF-like acting substance(s). Hence, it may be any substance, molecule, peptide, protein, protein-analogue, protein-variant, derivative, fragment, biopharmaceutical, inductor, precursor, prodrug, mutein, co-drug and/or any derivative, fragment and/or pharmaceutically acceptable salt of any of these. Therefore BDNF-like acting substance(s) may:
activates or is/are capable of activating a respective BDNF-receptor(s) of a cell. The BDNF-receptor(s) may for instance be the receptor of the LHs, peripheral T_{reg}s or PBMCs; and/or
results in or causes or is capable of resulting in or causing the generation of a BDNF and/or a BDNF-like acting substance(s).

Preferably, BDNF-like acting substance(s) activates or is capable of activating the respective BDNF-receptor(s). The activating or the capability of activating the respective BDNF-receptor(s) may be directly and/or it may be indirectly.

Preferably, the BDNF-like acting substance(s) activates or is/are capable of activating a respective BDNF-receptor(s) within the skin of the subject. The BDNF-like acting substance(s) may be any naturally occurring or artificial BDNF-like acting substance(s) as long as it has a sufficient biologic activity, i.e. an effective cross-reactivity in the subject, particularly is capable of activating the respective BDNF-receptor(s). Preferably, the BDNF-like acting substance(s) is known to the person skilled in the art at the effective date of filing of the present invention.

Preferably, the BDNF-like acting substance is any peptide, protein, protein-analogue, protein-variant or any derivative, fragment, biopharmaceutical, inductor, precursor, prodrug, mutein, co-drug and/or pharmaceutically acceptable salt of any of these.

In a preferred embodiment, the BDNF-like acting substance(s) may be as detailed below amongst others a BDNF and/or any derivative, fragment, protein-analogue, peptide, protein-variant, biopharmaceutical, inductor, precursor, prodrug, mutein, co-drug, propeptide thereof and/or any pharmaceutically acceptable salt of any of these.

More preferably, the BDNF-like acting substance(s) is an BDNF and/or any derivative, fragment, protein-analogue, peptide, protein-variant, biopharmaceutical, inductor, precursor, prodrug, mutein, co-drug, propeptide thereof and/or any pharmaceutically acceptable salt of any of these.

The expression “BDNF” as mentioned in any of the embodiments described herein preferably has to be interpreted broad. Typically, the BDNF activates or is are capable of activating a respective BDNF-receptor(s) of a cell. The BDNF-receptor(s) may for instance be the receptor of the LHs, peripheral T_{reg}s or PBMCs. Preferably, the activating or capability of activating is within the skin of the subject, more preferably locally in effective amounts within the skin of the subject, even more preferably not in amounts systemically effective within the subject's body. Preferably the BDNF may be any type of BDNF known to the person skilled in the art. Furthermore, the present invention encompass the use or medical use of precursors, prodrugs and propeptides of such BDNF which are usually not active as such, i.e. have no immunomodulatory activity, but which, upon use in accordance with the present invention, are converted into the actually active BDNF. Hence, these may be used in the method or the medical use as described herein or in preparation of the pharmaceutical compositions, injectable dosage form, topical dosage forms, medical devices and/or kit of parts as described herein.

Preferably, the BDNF may be any peptide, protein, protein-analogue, protein-variant or any derivative, fragment, biopharmaceutical, inductor, precursor, prodrug, mutein, co-drug and/or pharmaceutically acceptable salt of any of these.

The BDNF does not necessarily be derived from or be identical to the BDNF of the same genus and/or species the subject belongs to, it may be any naturally occurring or an artificial BDNF, as long as it has a sufficient biologic activity, i.e. an effective cross-reactivity in the subject, particularly is capable of activating the respective BDNF-receptor(s). Preferably, the biologic activity of the BDNF and/or BDNF-like acting substance(s) is sufficient to achieve the treating and/or preventing of the inflammatory disease, immunological disease and/or autoimmunological disease, preferably in the subject.

A BDNF and/or an BDNF-like acting substance typically affects cells by binding and activating the respective BDNF-receptor on the cells. Hence, the activating of the respective receptor typically leads to the affectation of the cell as described above for the immunomodulatory substance. Hence, any embodiment mentioned herein for the immunomodulatory substance relating to the affecting cells like LHs, peripheral T_{reg}s or PBMCs is independently and mutatis mutandis applicable to the BDNF and the BDNF-like acting substance.

The activating or the capability of activating the respective BDNF-receptor(s) by the BDNF and/or BDNF-like acting substance(s) may be directly and/or it may be indirectly.

If not mentioned otherwise, the description and definition of the cytokine(s) or the cytokine-like acting substance stated above is independently and mutatis mutandis applicable to the BDNF and/or BDNF-like acting substance(s). Particularly, if not mentioned otherwise, the description and definition of the cytokine(s) or the cytokine-like acting substance in respect of“directly” and “indirectly” activating or capable of activating the respective BDNF-receptor(s), is independently and mutatis mutandis applicable to the BDNF and/or BDNF-like acting substance(s).

Preferably, the BDNF and/or BDNF-like acting substance(s) for use according to the present invention and/or the BDNF and/or BDNF-like acting substance(s) of steps (B), (B₁) and/or (C) of the method, the pharmaceutical compositions, injectable dosage forms, topical dosage forms, medical devices and/or kits of parts, is capable of affecting and/or affects the LHs, peripheral T_{reg}s or PBMCs. Preferably, the PBMCs are naïve PBMCs, more preferably lymphocytes, even more preferably T-cells and/or B-cells, still more preferably naïve lymphocytes, still even more preferably naïve B-cells and/or naïve T-cells, further preferably naïve T-cells.

More preferably, the BDNF and/or BDNF-like acting substance(s) is capable of directly and/or indirectly affecting and/or affects the LHs, peripheral T_{reg}s or PBMCs.

More preferably, the BDNF and/or BDNF-like acting substance(s) is capable of directly and/or indirectly affecting and/or affects the LHs, peripheral T_{reg}s or PBMCs in the subject as defined in any of the embodiments according to the present invention, further preferably within the skin of the subject.

If not mentioned otherwise, the description and definition of the immunomodulatory substance(s) stated above is independently and mutatis mutandis applicable to the BDNF and/or BDNF-like acting substance(s). Particularly, if not mentioned otherwise, the description and definition of the immunomodulatory substance(s) in respect of “directly” and “indirectly” affecting or capable of affecting the PBMCs is independently and mutatis mutandis applicable to the BDNF and/or BDNF-like acting substance(s).

The BDNF and/or BDNF-like acting substance(s) for use according to the present invention and/or the BDNF and/or BDNF-like acting substance(s) of steps (B), (B₁) and/or (C) of the method, the pharmaceutical compositions, injectable dosage forms, topical dosage forms, medical devices and/or kits of parts, may be similar or identical, preferably identical, to any BDNF and/or BDNF-like acting substance(s) naturally occurring in a vertebrate, preferably a human or a mammal, more preferably in the subject as defined in any of the embodiments according to the present invention. Preferably, the BDNF and/or BDNF-like acting substance(s) may be similar or identical, preferably identical, to any peptide, protein or any fragment of these of BDNF and/or BDNF-like acting substance(s) naturally occurring in a vertebrate, preferably a human or a mammal as defined above, more preferably in the subject as defined in any of the embodiments according to the present invention.

The BDNF and/or BDNF-like acting substance(s) may comprise, preferably consist of an amino acid sequence which has an amino acid sequence identity to any peptide, protein or any fragment of an BDNF and/or BDNF-like acting substance(s) naturally occurring in a vertebrate, preferably a mammal, more preferably a human or a mammal animal as defined above, more preferably in the subject as defined in any of the embodiments according to the present invention. More preferably, the amino acid sequence identity is with reference to amino acid sequence SEQ ID NO: 4 or any fragment thereof. Preferably, the amino acid sequence identity is 70% or more, even more preferably 85% or more, still more preferably 90% or more, still even more preferably 95% or more, further preferably 97% or more, even further preferably 98% or more, still further preferably 99% or more and 100% or less. Preferably, the amino acid sequence identity includes substitutions, insertions and/or deletions of amino acid residues in respect to the naturally occurring peptide, protein or any fragment of these.

Preferably, the total number of substitutions, insertions and/or deletions or the total number combined of substitutions, insertions and/or deletions of amino acid residues is 60 or less, more preferably 40 or less, even more preferably 20 or less, still more preferably 12 or less, still even more preferably 8 or less, further preferably 4 or less, even further preferably 2 or less and zero or more, still further preferably zero, preferably with reference to amino acid sequence SEQ ID NO: 4, wherein more preferably the subject is a human.

The BDNF and/or BDNF-like acting substance(s) may be identical to an endogenous immunomodulatory substance(s) of the same genus and/or species the subject belongs to or the amino acid sequence identity refers to an endogenous immunomodulatory substance(s) of the same genus and/or species the subject belongs to. The expression “is identical” typically means in this particular context that the BDNF and/or BDNF-like acting substance(s) is in respect to its primary, secondary, tertiary and quaternary protein structure identical to an/the BDNF and/or BDNF-like acting substance(s) of the same genus and/or species the subject belongs to, more preferably, also in view of the modification pattern, particularly glycosylations.

The BDNF and/or BDNF-like acting substance(s) for use according to the present invention and/or the BDNF and/or BDNF-like acting substance(s) of steps (B), (B₁) and/or (C) of the method, the pharmaceutical compositions, injectable dosage forms, topical dosage forms, medical devices and/or kits of parts, may be derived from an individual belonging to the same genus and/or species as the subject, or which is identical to an/the BDNF and/or BDNF-like acting substance(s) of the same genus and/or species the subject belongs to. For instance, in case the subject is: a human the BDNF and/or BDNF-like acting substance(s) is human BDNF and/or BDNF-like acting substance(s), more preferably human BDNF and/or BDNF-like acting substance(s) comprising, even more preferably consisting of, preferably as effective amino acid sequence, the amino acid sequence SEQ ID NO: 4; a cow the BDNF and/or BDNF-like acting substance(s) is bovine BDNF and/or BDNF-like acting substance(s); a horse the BDNF and/or BDNF-like acting substance(s) is equine BDNF and/or BDNF-like acting substance(s); a donkey the BDNF and/or BDNF-like acting substance(s) is donkey BDNF and/or BDNF-like acting substance(s); an elephant the BDNF and/or BDNF-like acting substance(s) is elephant BDNF and/or BDNF-like acting substance(s); a sheep the BDNF and/or BDNF-like acting substance(s) is sheep BDNF and/or BDNF-like acting substance(s); a goat the BDNF and/or BDNF-like acting substance(s) is goat BDNF and/or BDNF-like acting substance(s); a pig the BDNF and/or BDNF-like acting substance(s) is porcine BDNF and/or BDNF-like acting substance(s); a rabbit the BDNF and/or BDNF-like acting substance(s) is rabbit BDNF and/or BDNF-like acting substance(s); a mouse the BDNF and/or BDNF-like acting substance(s) is mouse BDNF and/or BDNF-like acting substance(s); a rat the BDNF and/or BDNF-like acting substance(s) is rat BDNF and/or BDNF-like acting substance(s); a camel the BDNF and/or BDNF-like acting substance(s) is camel BDNF and/or BDNF-like acting substance(s); a dromedary the BDNF and/or BDNF-like acting substance(s) is dromedary BDNF and/or BDNF-like acting substance(s); a lama the BDNF and/or BDNF-like acting substance(s) is lama BDNF and/or BDNF-like acting substance(s); an alpaca the BDNF and/or BDNF-like acting substance(s) is alpaca BDNF and/or BDNF-like acting substance(s); a dog the BDNF and/or BDNF-like acting substance(s) is dog BDNF and/or BDNF-like acting substance(s); and/or a cat the BDNF and/or BDNF-like acting substance(s) is cat BDNF and/or BDNF-like acting substance(s).

Preferably, the BDNF and/or BDNF-like acting substance(s) for use according to the present invention and/or the BDNF and/or BDNF-like acting substance(s) of steps (B), (B₁) and/or (C) of the method, the pharmaceutical compositions, injectable dosage forms, topical dosage forms, medical devices and/or kits of parts, is not the subject's endogenous BDNF and/or BDNF-like acting substance(s). In other words, preferably the BDNF and/or BDNF-like acting substance(s) is not an BDNF and/or BDNF-like acting substance(s) isolated from the subject's body, i.e. from the individual subject to be treated or prevented.

Preferably, the BDNF and/or BDNF-like acting substance(s) for use according to the present invention and/or the BDNF and/or BDNF-like acting substance(s) of steps (B), (B₁) and/or (C) of the method, the pharmaceutical compositions, injectable dosage forms, topical dosage forms, medical devices and/or kits of parts, may be, preferably, is a recombinant BDNF and/or BDNF-like acting substance(s) which may be generated by any recombinant expression techniques known to the person skilled in the art, a chemically synthesized BDNF and/or BDNF-like acting substance(s) synthesized by any production techniques known to the person skilled in the art, an artificially produced BDNF and/or BDNF-like acting substance(s) produced by any production techniques known to the person skilled in the art, a naturally occurring BDNF and/or BDNF-like acting substance(s) obtained from natural sources like an animal or human, or any combination thereof. More preferably, the BDNF and/or BDNF-like acting substance(s) is a recombinant BDNF and/or BDNF-like acting substance(s), chemically synthesized BDNF and/or BDNF-like acting substance(s), artificially produced BDNF and/or BDNF-like acting substance(s) or any combination thereof, even more preferably a recombinant BDNF and/or BDNF-like acting substance(s), still even more preferably recombinant human BDNF-like acting substance(s), further preferably BDNF-like acting substance(s), produced in genetically modified Escherichia coli (produced and commercially available from R&D Systems under the name “Recombinant Human BDNF Protein”, catalogue number 248-BDB/CF [carrier free]).

Furthermore, the BDNF and/or BDNF-like acting substance(s) for use according to the present invention and/or the BDNF and/or BDNF-like acting substance(s) of steps (B), (B₁) and/or (C) of the method, the pharmaceutical compositions, injectable dosage forms, topical dosage forms, medical devices and/or kits of parts, may or may not be derivatized, stabilized, fused with other proteins or peptides, conjugated with polymers, contain amino acid analogue(s) or artificial amino acids, modified, covalently or non-covalently, for instance glycosylated or methylated by e.g. posttranslational modification and/or oligomerized, e.g. dimerized or trimerized as long as it is capable of affecting and/or affects LHs, peripheral T_{reg}s or PBMCs. More preferably, the modification pattern and/or oligomerization status is similar or identical to the peptide, protein or any fragment of these naturally occurring in a vertebrate, preferably a mammal, more preferably a human or a mammal animal as defined above, more preferably in the subject as defined in any of the embodiments according to the present invention.

Preferably, in any of the embodiments described herein it is intended to administer the BDNF and/or BDNF-like acting substance(s) in an amount low enough to avoid a systemic increase of the BDNF and/or BDNF-like acting substance(s)-concentration in the subject's body. Hence, the BDNF and/or BDNF-like actin substance(s) may be administered in an amount that, when scaled to the entire body of the subject, the resulting systemic concentrations remain in the magnitude of the endogenous levels. Thereby, without wishing to be bound to theory, it is believed that the generation of an increased BDNF and/or BDNF-like acting substance(s)-concentration for instance at sites of inflammation, like inflamed joint. As already stated above, for instance naïve T-cells can mature in the presence of antigen/autoantigen/allergen towards cytotoxic, which may act pro-inflammatory. Thereby decreasing, cancelling or even reversing the beneficial effects achieved by the present invention, particularly, the anti-inflammatory effect of the regulatory T-cells and/or helper T-cells, or of a subset thereof, which are presumably generated.

Preferably, in any of the embodiments described herein, particularly in step (B), (B₁) and/or (C), the BDNF and/or BDNF-like acting substance(s) is administered in an amount that does not cause a systemic increase, more preferably an effective systemic increase, of the concentration of BDNF and/or BDNF-like acting substance(s) in the subject, preferably the blood of the subject, and/or that causes a systemic increase of the concentration of the BDNF and/or BDNF-like acting substance(s). Preferably, BDNF and/or BDNF-like acting substance(s) is administered in an amount low enough that the systemic increase is non-effective.

Preferably, in any of the embodiments described herein, particularly in step (B), (B₁) and/or (C), the BDNF and/or BDNF-like acting substance(s) is administered in an amount that causes a local increase, preferably an effective local increase, of the concentration of the BDNF and/or BDNF-like acting substance(s) in the subject, preferably in the skin tissue of the subject. The skin tissue is preferred, because unlike administration into the blood, no or less flushing away and dilution of the BDNF and/or BDNF-like acting substance(s) takes place.

Preferably, in any of the embodiments described herein, particularly in step (B), (B₁) and/or (C), the generated increased concentration, preferably the generated effective increased concentration of the BDNF and/or BDNF-like acting substance(s) administered is only local but not systemic in the subject.

Preferably, in any of the embodiments described herein, particularly in step (B), (B₁) and/or (C), the BDNF and/or BDNF-like acting substance(s) is administered in an amount that does not cause a systemic activation, preferably an effective systemic activation, of the respective receptor of the BDNF and/or BDNF-like acting substance(s) of a cell, and/or that does not cause a systemic generation, preferably an effective systemic generation, of the BDNF and/or BDNF-like acting substance(s), preferably in the subject.

Preferably, in any of the embodiments described herein, particularly in step (B), (B₁) and/or (C), the BDNF and/or BDNF-like acting substance(s) is administered in an amount that causes a local activation, preferably an effective local activation of the respective receptor of the BDNF and/or BDNF-like acting substance(s) of a cell, and/or causes a local generation, preferably an effective local generation of the BDNF and/or BDNF-like acting substance(s), preferably in the subject. Preferably, the activation and/or generation, more preferably the effective generation and/or effective activation is only local but not systemic in the subject.

Preferably, in any of the embodiments described herein, particularly in step (B), (B₁) and/or (C), the BDNF-like acting substance(s), preferably BDNF, is administered in an effective amount, preferably an effective total amount.

Preferably, in any of the embodiments described herein, particularly in step (B), (B₁) and/or (C) the BDNF-like acting substance(s) is administered in an amount, preferably a total amount, equivalent in activity to 10 ng BDNF/kg body mass of the subject or less, preferably per administration dose, to the skin of the subject. More preferably, BDNF is administered in an amount equivalent in activity to 5 ng BDNF/kg or less, even more preferably 2.5 ng BDNF/kg or less, still more preferably 1 ng BDNF/kg or less, still even more preferably 0.5 ng BDNF/kg or less, further preferably 0.3 ng BDNF/kg or less, still even more preferably 0.1 ng BDNF/kg or less, further preferably 0.05 ng BDNF/kg or less. There is no lower limit for the amount, preferably the total amount, per kg body mass as long as the beneficial effects can be achieved, however, usually, a lower limit is and amount equivalent in activity to 0.001 ng BDNF/kg or more, more preferably 0.005 ng BDNF/kg or more, even more preferably 0.007 ng BDNF/kg or more, still more preferably 0.01 mg/kg or more. More preferably, the BDNF-like acting substance(s) is administered in an amount, preferably a total amount, in the range of equivalent in activity to 10 ng BDNF/kg or less and 0.001 ng BDNF/kg or more, even more preferably ng BDNF/kg or less and 0.001 ng BDNF/kg or more, still more preferably 2.5 ng BDNF/kg or less and 0.005 ng BDNF/kg or more, still even more preferably 1 ng BDNF/kg or less and 0.005 ng BDNF/kg or more, further preferably 0.5 ng BDNF/kg or less and 0.007 ng BDNF/kg or more, even further preferably 0.3 ng BDNF/kg or less and 0.007 ng BDNF/kg or more, still further preferably 0.1 ng BDNF/kg or less and 0.01 ng BDNF/kg or more, still even further preferably 0.05 ng BDNF/kg or less and 0.01 ng BDNF/kg or more.

Preferably, in any of the embodiments described herein, particularly in step (B), (B₁) and/or (C), the BDNF-like acting substance(s) is administered, preferably per administration dose, in an amount, preferably a total amount, equivalent in activity to 500 ng BDNF or less, more preferably 100 ng BDNF or less, even more preferably 50 ng BDNF or less, still more preferably 25 ng BDNF or less, still even more preferably 10 ng BDNF or less, further preferably 5 ng BDNF or less. There is no lower limit for the amount, preferably the total amount, as long as the beneficial effects can be achieved, however, usually, a lower limit is an amount equivalent in activity to 0.005 ng BDNF or more, more preferably 0.01 ng BDNF or more, even more preferably 0.05 ng BDNF or more, still even more preferably 0.1 ng BDNF or more, further preferably 0.2 ng BDNF or more. More preferably, the BDNF-like acting substance(s), is administered, preferably per administration dose, in an amount, preferably a total amount, in the range of equivalent in activity to 500 ng BDNF or less and 0.005 ng BDNF or more, even more preferably 100 ng BDNF or less and 0.01 ng BDNF or more, still more preferably 50 ng BDNF or less and 0.05 ng BDNF or more, still even more preferably 25 ng BDNF or less and 0.05 ng BDNF or more, further preferably 10 ng BDNF or less and 0.1 ng BDNF or more, even further preferably 5 ng BDNF or less and 0.2 ng BDNF or more.

Preferably, in any of the embodiments described herein, particularly in step (B), (B₁) and/or (C), BDNF substance(s) is administered in an amount, preferably a total amount, of 10 ng/kg body mass of the subject or less, preferably per administration dose, to the skin of the subject. More preferably, BDNF is administered in an amount of 5 ng/kg or less, even more preferably 2.5 ng/kg or less, still more preferably 1 ng/kg or less, still even more preferably 0.5 ng/kg or less, further preferably 0.3 ng/kg or less, still even more preferably 0.1 ng/kg or less, further preferably 0.05 ng/kg or less. There is no lower limit for the amount, preferably the total amount, per kg body mass as long as the beneficial effects can be achieved, however, usually, a lower limit is 0.001 ng/kg or more, more preferably 0.005 ng/kg or more, even more preferably 0.007 ng/kg or more, still more preferably 0.01 mg/kg or more. More preferably, BDNF is administered in an amount, preferably a total amount, in the range of 10 ng/kg or less and 0.001 ng/kg or more, even more preferably ng/kg or less and 0.001 ng/kg or more, still more preferably 2.5 ng/kg or less and 0.005 ng/kg or more, still even more preferably 1 ng/kg or less and 0.005 ng/kg or more, further preferably 0.5 ng/kg or less and 0.007 ng/kg or more, even further preferably 0.3 ng/kg or less and 0.007 ng/kg or more, still further preferably 0.1 ng/kg or less and 0.01 ng/kg or more, still even further preferably 0.05 ng/kg or less and 0.01 ng/kg or more.

Preferably, in any of the embodiments described herein, particularly in step (B), (B₁) and/or (C), the BDNF is administered, preferably per administration dose, in an amount, preferably a total amount, of 500 ng or less, more preferably 100 ng or less, even more preferably 50 ng or less, still more preferably 25 ng or less, still even more preferably 10 ng or less, further preferably 5 ng or less. There is no lower limit for the amount, preferably the total amount, as long as the beneficial effects can be achieved, however, usually, a lower limit is 0.005 ng or more, more preferably 0.01 ng or more, even more preferably 0.05 ng or more, still even more preferably 0.1 ng or more, further preferably 0.2 ng or more. More preferably, BDNF is administered, preferably per administration dose, in an amount, preferably a total amount, in the range of 500 ng or less and 0.005 ng or more, even more preferably 100 ng or less and 0.01 ng or more, still more preferably 50 ng or less and 0.05 ng or more, still even more preferably 25 ng or less and 0.05 ng or more, further preferably 10 ng or less and 0.1 ng or more, even further preferably 5 ng or less and 0.2 ng or more.

Preferably, the BDNF-like acting substance(s) as mentioned in any of the embodiments described herein, specifically the immunomodulatory substance(s) for use according to the present invention and/or the immunomodulatory substance(s) of steps (B), (B₁) and/or (C) of the method, the pharmaceutical compositions, injectable dosage forms, topical dosage forms, medical devices and/or kits of parts, is BDNF.

As can be seen from the examples, BDNF has amongst others the beneficial effects of very effectively relieving the fatigue in case of multiple sclerosis and the subject's condition is stabilized or even improved as indicated by a decreasing SHILD-score.

In any of the embodiments described herein the amount, that is any of the amount per kg body mass, the total amount per kg body mass, the amount and/or the total amount, may be administered in a single administration dose or distributed to a plurality of administration doses as long as the indicated amounts or total amounts, respectively, are administered. However, a single administration dose is preferred. Preferably, such amounts, whether as single administration dose or if distributed to a plurality of administration doses, is administered within 8 hours or less, more preferably within 6 hours or less, even more preferably within 3 hours or less, even more preferably within 1.5 hour or less, still more preferably within 1 hour or less, still even more preferably within 0.5 hours or less, further preferred within 15 min or less, even further preferably within 5 min (minute) or less, still further preferably within 1 min or less. There is no lower limit for the period of time within which such amounts might be administered. However, it may be in the interest of the treated subject to keep the period of time as short as possible. Preferably the method is performed within 8 hours or less and 0.05 sec (second(s)) or more, more preferably within 6 hours or less and 0.5 sec or more, even more preferably within 3 hour or less and 1 sec or more, still more preferably within 1.5 hour or less and 1 sec or more, still even more preferably within 1 hour or less and 1 sec or more, further preferably within 0.5 hours or less 1 sec or more, even further preferred within 15 min or less 1 sec or more, still further preferably within 5 min or less and 2 sec or more, preferably in relation to step (A), more preferably before and/or after performing step (A). In case of a plurality of administration doses, these may be administered in any order, successively, simultaneously, separately, combined together or any combination thereof. A plurality of administration doses may for instance occur in case repetitions of steps are performed, e.g. step (A) and/or step (B), as mentioned in any of the embodiments described herein.

The expression “separately” as mentioned in any of the embodiments described herein is preferably to be understood in terms of temporal, spatial and/or in terms of the formulation, i.e., in case of multiple substances, they may be formulated separately in several formulations and/or may be administered spatial and/or in time separately.

The expression “combined together” as mentioned in any of the embodiments described herein is preferably to be understood in terms of temporal, spatial and/or in terms of the formulation, i.e., in case of multiple substances, they may be formulated together in one single formulation (composition, plaster, time-release plaster) and/or may be administered in combination with each other, that is the administration is spatially and/or in time concomitantly.

Preferably, in any of the embodiments described herein the amount, that is any of the amount per kg body mass, the total amount per kg body mass, the total amount and/or the total amount, is administered at a frequency of once per day or less often, more preferably once per 2 days or less often, even more preferably once per 3 days or less often, still more preferably once per 4 days or less often, still even more preferably once per 5 days or less often, further preferably once per week or less often, even further preferably once per 2 weeks or less often, still further preferably once per month or less often. Usually, there is no lower limit for the frequency as long the beneficial effects can be achieved or is maintained. Furthermore, it may be in the interest of the treated subject and in respect of effort and cost efficiency to administer such amounts as seldom as possible. Nevertheless, a lower limit may be, preferably, is that the administration of such amounts is performed at a frequency of once per two months or more often, more preferably, once per 6 weeks or more often. Preferably, the method is performed at a frequency of once per day to one per two months, more preferably once per 3 days to once per 6 weeks, even more preferably once per 5 days to once per month.

Preferably, step (A) and step (B) as mentioned in any of the embodiments described herein may be performed in any order, successively, simultaneously, separately, combined together or any combination thereof.

Preferably, step (A) and step (C) as mentioned in any of the embodiments described herein may be performed in any order, successively, simultaneously, separately, combined together or any combination thereof.

Preferably, step (A), step (B) and step (C) as mentioned in any of the embodiments described herein may be performed in any order, successively, simultaneously, separately, combined together or any combination thereof.

Preferably, step (A), step (B) and step (B₁) as mentioned in any of the embodiments described herein may be performed in any order, successively, simultaneously, separately, combined together or any combination thereof, with the exception that preferably step (B) and step (B₁) are performed separately and/or not combined together. Step (B) and step (B₁) are particularly performed separately and/or are not combined together in case that in step (B) IL-4 and in step (B₁) BDNF is administered.

Preferably, step (A), step (B), step (B₁) and step (C) as mentioned in any of the embodiments described herein may be performed in any order, successively, simultaneously, separately, combined together or any combination thereof, with the exception that preferably step (B) and step (B₁) are performed separately and/or not combined together. Step (B) and step (B₁) are particularly performed separately and/or are not combined together in case that in step (B) IL-4 and in step (B₁) BDNF is administered.

Preferably, in step (A), step (B₁) and step (C) as mentioned in any of the embodiments described herein may be performed in any order, successively, simultaneously, separately, combined together or any combination thereof.

Preferably, step (A), step (B) and step (C) of the first set of steps, and step (A), step (B₁) and step (C) of the second set of steps as mentioned in any of the embodiments described herein may be performed in any order, successively, simultaneously, separately, combined together or any combination thereof.

In case in step (B) two or more immunomodulatory substances are administered, for instance IFN-γ and IL-4, IL-4 and BDNF or IFN-γ, IL-4 and BDNF, it is to be understood that such substances may be administered successively, simultaneously, separately, combined together or in any combination thereof.

For instance, in case of an injection or a topical application, the compositions, topical or injectable dosage forms may contain both, the immunomodulatory substance(s) of step (B) and of step (C). Hence, in this example, with a single administration step, steps (B) and (C) can be realized combined together and simultaneously. In case of for instance a time-release plaster which comprises the immunomodulatory substances of step (B) and of step (C), the immunomodulatory substance(s) of step (B) may be administered before the immunomodulatory substance(s) of step (C). Hence, in this example, with a single administration step, steps (B) and (C) can be realized combined together but are administered successively. Hence, ‘combined together’ includes successively and simultaneously.

Furthermore, any of steps (A), (B), (B₁) and/or (C) and/or any combined steps of these may or may not be completed before the next step is performed or any combination thereof. Hence, the administration of any of these steps may still be performed while the administration of another of these steps is started or completed or any combination thereof.

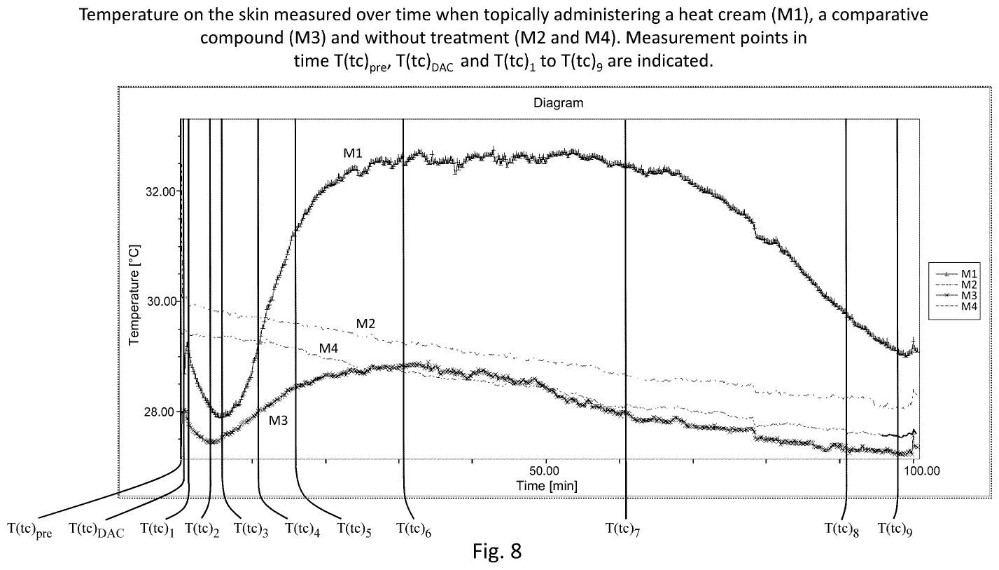

Preferably, the term “generating” is to be understood to encompass ‘initiating’ and/or ‘establishing’. For instance, the term “generating” in any of steps (A-0), (A-1), (A-2), (A-3), (A-4) and (A-5) described below, encompasses that the accumulation of PBMCs, vasodilation, increased blood volume, increased sO₂, increased rHb, increased temperature and/or redness may be initiated and:
the accumulation of PBMCs, vasodilation, increased blood volume, increased sO₂, increased rHb, increased temperature and/or redness may be established immediately, for instance within 0 sec to 5 sec, more preferably within 1 sec to 5 sec; and/or
the accumulation of PBMCs, vasodilation, increased blood volume, increased sO₂, increased rHb, increased temperature and/or redness may be established with a delay or time-shift of for instance 5 sec or more up to 3 hours, more preferably 5 sec or more up to 1.5 hours, even more preferably 5 sec or more up to 1 hour, still more preferably 10 sec or more up to 0.75 hours, still even more preferably 10 sec or more up to 0.5 hours, still further preferably up to 10 sec or more up to 0.25 hours (see for instance FIG. 8 in case of the temperature increase on the skin when using a heat crème); and/or
before the delayed or time-shifted accumulation of PBMCs, vasodilation, increased blood volume, increased sO₂, increased rHb, increased temperature and/or redness is established, a diminished accumulation, vasodilation, blood volume, temperature and/or redness may precede (see for instance FIG. 8 in case of the temperature increase on the skin when using a heat crème); and/or
the accumulation of PBMCs, vasodilation, increased blood volume, increased sO₂, increased rHb, increased temperature and/or redness may or may not be established before the next step is performed. Hence, the next step can be performed even if the accumulation, vasodilation, increased blood volume, increased sO₂ and/or increased rHb, increased temperature and/or redness is not or not yet fully established.

However, preferably, in any of the embodiments described herein it is to be understood that the individual effects generated by steps (A), (B), (B₁) and/or (C) at least partially and/or totally overlap in time as well as spatially, for instance e.g. to be mutually dependent, supportive, interactive, complementary, additive, synergetic and/or to act by any other way together. Hence, it is to be understood that the method should be performed accordingly.

Hence, preferably, in any of the embodiments described herein the method is performed in such a way that the individual effects generated by steps (A), (B), (B₁) and/or (C) at least partially and/or totally overlap in time and/or spatially.

Hence, preferably, in any of the embodiments described herein, the effect generated by step (A), particularly the accumulation of PBMCs, vasodilation, increased blood volume, increased sO₂, increased rHb, increased temperature and/or redness, partially and/or totally overlaps in time and/or spatially with the presence of the immunomodulatory substance(s), preferably the increased concentration and/or the amount of immunomodulatory substance(s) within the skin generated by administering the immunomodulatory substance(s) in step (B), (B₁) and/or (C). Preferably, the immunomodulatory substance(s), preferably the increased concentration and/or the amount of the immunomodulatory substance(s), present within the skin is an effective concentration and/or an effective amount of the immunomodulatory substance(s). It is noted that such amount may vary between individual subjects. Similarly, for instance the overlap in time may vary from subject to subject.

Preferably, in any of the embodiments described herein, the overlap in time, whether it is a partial or total overlap, is sufficient to achieve the beneficial effects and may vary between individual subjects. More preferably it is 2 min or more, even more preferably 5 min or more, still more preferably 10 min or more, still even more preferably 15 min or more, further preferably 20 min or more, even further preferably 0.5 hours or more, still further preferably 0.75 hours or more, still even further preferably 1 h or more. There is no upper limit for the overlap in time, however, usually it is 48 hours or less. Preferably, the overlap in time, whether it is a partial or total overlap, is in the range of 2 min or more and 48 hours or less, more preferably 5 min or more and 24 hours or less, even more preferably 10 min or more 24 hours or less, still more preferably 15 min or more 12 hours or less, still even more preferably 20 min or more 12 hours or less, further preferably 0.5 hours or more and 6 hours or less, even further preferably 0.75 hours or more and 3 hours or less, still more preferably 1 h or more and 3 hours or less.

Preferably, in any of the embodiments described herein, the accumulation of PBMCs is present, sustains and/or is maintained for a duration of time of 2 min or more, more preferably 10 min or more, even more preferably for 0.25 hours or more. There is no upper limit for such persistence and/or maintenance, however, usually it is for a duration of time of 48 hours or less, more preferably 6 hours or less, even more preferably 3 hours or less, still more preferably 1.5 hour or less, still even more preferably 1 hour or less, further preferably 0.75 hours or less. Preferably, the persistence and/or maintenance is for a duration of time in the range of 10 min or more and 48 hours or less, more preferably 0.25 hours or more and 48 hours or less, even more preferably 0.25 hours or more and 6 hours or less, still more preferably 0.5 hours or more and 3 hours or less, still even more preferably 0.75 hours or more and 1.5 hours or less.

Preferably, in any of the embodiments described herein, the increased blood volume, vasodilation, increased sO₂, increased rHb, increased temperature and/or redness is present, sustains and/or is maintained for a duration of time of 2 min or more, more preferably 10 min or more, even more preferably for 0.25 hours or more. There is no upper limit for the persistence of the increased temperature on the skin, however, usually it is for a duration of time of 6 hours or less, more preferably 3 hours or less, even more preferably 1.5 hour or less, still more preferably 1 hour or less, still even more preferably 0.75 hours or less. Preferably the increased temperature on the skin is present, sustains and/or is maintained for a duration of time in the range of 2 min or more and 6 hours or less, more preferably 5 min or more and 3 hours or less, even more preferably 10 min or more and 1.5 hour or less, still more preferably 0.25 hours or more and 1 hour or less, still even more preferably 0.25 hours or more and 0.75 hours or less.

The duration of time may be continuous or with interruptions, i.e. distributed to two or more sub-durations. Therefore, the duration of time is preferably the total duration of time which may be composed of a single continuous duration or the sum of two or more sub-durations.

Furthermore, the duration of time is preferably the total duration of time the effect generated by step (A) is present and/or is maintained after performing any or all of steps (B), (B₁) and/or (C) and/or after the effect of any or all of steps (B), (B₁) and/or (C) is generated or effected, for instance within 15 hours or less, preferably within 10 hours or less, more preferably within 8 hours or less, even more preferably within 6 hours or less, even more preferably within 2 hours or less and e.g. concomitantly or immediately after performing any or all of steps (B), (B₁) and/or (C). The accumulation of PBMCs, vasodilation, increased blood volume, increased sO₂, increased rHb, increased temperature and/or redness may for instance be present and/or maintained for the total duration of time by performing step (A) multiple times, e.g. by administering a temperature-increasing agent to the skin a time-shifted a second time or by applying a heat pad to the skin a time-shifted second time. However, it is noted that this does not exclude that step (A) is performed before or the effect thereof is for instance generated before any or all of steps (B), (B₁) and/or (C) are performed.

Furthermore, the duration or total duration of time for which the effect generated by step (A) is present and/or is maintained after performing any or all of steps (B), (B₁) and/or (C). Such the duration of time is for instance from at the same time when performing or immediately after performing any or all of steps (B), (B₁) and/or (C) to 15 hours or less, preferably 10 hours or less, more preferably 8 hours or less, even more preferably 6 hours or less, even more preferably 2 hours or less. Hence, the effect generated by step (A), that is the accumulation of PBMCs, vasodilation, increased blood volume, increased sO₂, increased rHb, increased temperature and/or redness, may for instance be present and/or maintained for the total duration of time by performing step (A) multiple times, e.g. by applying a heat pad several times to the skin or using a continuously warming heat patch. However, it is noted that this does not exclude that step (A) is performed before any or all of steps (B), (B₁) and/or (C) are performed.

Usually, in step (B), (B₁) and/or (C) as mentioned in any of the embodiments described herein, the presence of the immunomodulatory substance(s), that is the increased concentration and/or the amount of the immunomodulatory substance(s), within the skin is for a duration of time of 2 min or more, more preferably 10 min or more, even more preferably for 0.25 hours or more. There is no upper limit, however, usually it is for a duration of time of 24 hours or less, preferably 12 hours or less more preferably 6 hours or less, even more preferably 3 hours or less, still more preferably 1.5 hours or less, still even more preferably 1 hour or less, further preferably 0.75 hours or less. Preferably, the presence of the immunomodulatory substance(s) is for a duration of time from immediately up to 24 hour, more preferably up to 12 hours, even more preferably immediately up to 6 hours, still more preferably from immediately up to 3 hours, still even more preferably 5 sec up to 1.5 hours, further preferably 5 sec up to 1 hour, even further preferably 10 sec up to 0.75 hours.

Preferably, the skin in all embodiments of the present invention described herein comprises, preferably consists of, in skin thickness direction of the skin surface and one or more skin layers. The one or more skin layers comprise, preferably consist of, the epidermis, comprising stratum disjunctum, stratum corneum, stratum lucidum, stratum granulosum, stratum spinosum and stratum basale; the dermis comprising a papillary region or papillary dermis and a reticular dermis; and the subcutis.

The expression “skin thickness direction” as mentioned in any of the embodiments described herein refers to the direction perpendicular to the skin's surface, preferably when seen from the skin surface towards the bones.

The expression “underneath” as mentioned in any of the embodiments described herein, when used with reference to the skin or skin layers, defines a position located closer to the bones and hence further away from the skin surface.

The expression “above” as mentioned in any of the embodiments described herein, when used with reference to the skin or skin layers, defines a position located further away from the bones and hence closer to the skin surface.

The expression “sub-topical layer” or “sub-topical layers” of the skin as mentioned in any of the embodiments described herein refers to the layers of the skin comprising, preferably consisting of, the epidermis, dermis and subcutis. More preferably, comprising, even more preferably consisting of, the stratum spinosum, stratum basale, dermis and subcutis.

The expression “skin tissue” as mentioned in any of the embodiments described herein refers to the skin excluding any blood vessels like capillaries and their vessel/capillary lumen. For instance, the expression “skin tissue of a sub-topical layer” as used in the present invention refers to a sub-topical layer of the skin excluding any blood vessels like capillaries and their vessel/capillary lumen.

Preferably, skin and/or the layer(s) of the skin have an expansion in the thickness direction and an expansion extending parallel to a surface of the skin and/or of the layer(s).

The expression “expansion” as mentioned in any of the embodiments described herein refers to a linear, one-dimensional expansion of the skin and/or the layer(s) extending in parallel to the surface of the skin or in thickness direction of the skin.

An expansion parallel to the surface of the skin, as mentioned in any of the embodiments described herein, is termed “surface parallel expansion”. It is preferably expressed in cm (centimetre(s)). It may refer to an expansion in the length or the width.

An expansion in thickness direction of the skin and/or of the layer(s), as mentioned in any of the embodiments described herein, is termed “thickness”. The thickness is preferably expressed in cm (centimetre(s)) and/or by stating one or more of the layers of the skin as defined above. The thickness of the skin and/or the layer(s) may vary in dependency of the genus and/or species a subject belongs to. Usually, in case the subject is a human, the skin has a thickness of 6 mm (millimetre(s)) or less, preferably 5 mm or less.

Preferably, in any of the embodiments described herein, the skin is of a skin area. In other words, the skin has an area designated as skin area. More preferably, in steps (A), (B), (B₁) and/or (C) the generating is within or on the skin of the skin area and the administering is to the skin of the skin area. That is, the generating or administering takes place at the skin of the skin area. Preferably, in any of the embodiments described herein, the administration of steps (B), (B₁) and/or (C) is effected partially and/or totally in the same area of the skin where the generating and/or administering of step (A) is effected.

Preferably, in any of the embodiments described herein, steps (B), (B₁) and/or (C) are performed partially and/or totally within the same area of the skin where step (A) is performed.

More preferably, in any of the embodiments described herein, in step (A) the skin is of a skin area [a], in step (B) the skin is of a skin area [b], in step (B₁) the skin is of a skin area [b₁] and/or in step (C) the skin is of a skin area [c].

If not mentioned otherwise, it is to be understood that any embodiment or definition described herein in terms of the ‘skin area’ in general is independently and mutatis mutandis applicable to the skin area [a], skin area [b], skin area [b₁] and/or skin area [c] as mentioned in any of the embodiments described herein.

The expression “skin area” as mentioned in any of the embodiments described herein refers to a two-dimensional expansion extending in parallel to the surface of the skin. Preferably, the skin area refers to the area in whose associated skin the effect of any of steps (A), step (B), step (B₁) and/or step (C) is achieved, particularly the generating of the accumulation of PBMCs, vasodilation, increased blood volume, increased sO₂, increased rHb, increased temperature and/or redness or the administering of the immunomodulatory substance(s) and/or skin-conditioning agent is achieved, that is the concentration of such substance(s) and/or agents is increased. That is, the generating or administering takes place within or to the skin of the skin area. The skin area is preferably indicated in m² (square meter(s)), cm² (square centimetre(s)) and/or mm² (square millimetre(s)).

The skin area, specifically the skin area [a], [b], [b₁] and/or [c], may independently have any two-dimensional shape as long as the beneficial effects can be achieved and may have a certain localisation on the subject's body. For instance, the skin area may have a circular, triangular, rectangular, square, elliptic and/or polygonal shape, the shape may be, preferably, is irregular, lengthy and/or compact (the outline is rather short in respect of the encompassed skin area, e.g. a square a shorter outline than a rectangle in respect of the encompassed area), it may have recesses, the skin area can be divided into several individual areas and/or have an outline with concave and/or convex parts or any combination of these. Preferably, the skin area is approximately circular, square or rather compact in shape and more preferably does not have recesses is divided into server individual areas.

The skin area, specifically the skin area [a], [b], [b₁] and/or [c], may independently have any size sufficient to achieve the beneficial effects and may have even a size of 1.5 m² or less. Preferably, the skin area is 1,000 cm² or less, more preferably 500 cm² or less, even more preferably 100 cm² or less, still more preferably 50 cm² or less, still even more preferably 25 cm² or less, further preferably 10 cm², even further preferably 5 cm² or less. Usually, the skin area is not smaller than 0.2 cm². Hence, a lower limit for the skin area is preferably 0.2 cm² or more, more preferably 0.5 cm² or more, even more preferably 1 cm² or more, still more preferably 2 cm² or more. Preferably, the skin area is in the range of 1.5 m² or less and 0.2 cm² or more, more preferably 1,000 cm² or less and 0.2 cm² or more, even more preferably 500 cm² or less and 0.5 cm² or more, still more preferably 100 cm² or less and 0.5 cm² or more, still even more preferably 50 cm² or less and 1 cm² or more, further preferably 25 cm² or less and 1 cm² or more, even further preferably 10 cm² or less and 2 cm² or more. The skin area as mentioned in any of the embodiments is preferably measured as described in the Materials and Methods' section ‘Measurement of areas and distances relating to the skin’.

The expression “skin of a/the skin area”, “skin layer(s) of a/the skin area” or the like as mentioned in any of the embodiments described herein refers to the fragment of the skin or of one or more skin layers as defined above, covered and/or outlined by the skin area. For instance, the expression ‘subcutis of the skin area’ refers to the fragment of the subcutis covered and/or outlined by the skin area. For instance, the expression ‘dermis of the skin area’ refers to the fragment of the dermis covered and/or outlined by the skin area. The expression “on a/the skin” as mentioned in any of the embodiments described herein typically refers to a topical treatment, administration, generation, application etc. performed on the surface of the skin, preferably the fragment of the skin which skin is covered and/or outlined by the skin area and/or the application area.

The expression “topical” as mentioned in any of the embodiments described herein typically refers to procedures like a treatment, administration, generation, application, delivery etc. which is performed on a body surface. These procedures may be performed on any particular place on an inner or outer body surface. Preferably, they are performed on the skin like, more preferably epicutaneously meaning a performance directly on the skin. Preferably, the expression “topical” excludes invasive procedures. Preferably, a topical administration, application and/or delivery of the immunomodulatory substance(s) as mentioned in any of the embodiments described herein is capable to administer, apply and/or delivery these substances into a sub-topical layer of the skin, more preferably into the epidermis, dermis and/or subcutis, even more preferably the dermis, of the skin and/or 6 mm or less and 0.05 mm or more, more preferably 4 mm or less and 0.3 mm or more, even more preferably 3 mm or less and 0.5 mm or more in skin thickness direction into the skin, when measured from the skin surface, preferably when measured perpendicular to the skin surface towards the bones.

The expressions “into a/the skin” and/or “within a/the skin” as mentioned in any of the embodiments described herein typically refers to an invasive treatment, procedure, administration, generation, application, delivery etc., for instance by injection, performed into the skin like into a sub-topical layer of the skin, for instance into the epidermis, dermis and/or subcutis, even more preferably the dermis.

The expression “injection” or “injected” as mentioned in any of the embodiments described herein typically refers to invasive procedures like treatment, administration, generation, application, delivery etc. performed into the skin. Preferably, the expression “injection” or “injected” excludes non-invasive procedures. Preferably, an injection as mentioned in any of the embodiments described herein is placed into a sub-topical layer of the skin, more preferably, into the epidermis, dermis and/or subcutis, even more preferably the dermis of the skin and/or the injection is preferably placed 6 mm or less and 0.05 mm or more, more preferably 4 mm or less and 0.3 mm or more, even more preferably 3 mm or less and 0.5 mm or more in skin thickness direction into the skin, when measured from the skin surface, preferably when measured perpendicular to the skin surface towards the bones.

The expression “to a/the skin” or “at a/the skin” as mentioned in any of the embodiments described herein preferably is the generic term covering the meaning of all three expressions “on a/the skin”, “into a/the skin” and/or “within a/the skin”, that is typically refers to a topical and/or invasive treatment, procedure, administration, generation, application etc., for instance by injection, performed into the skin like a sub-topical layer of the skin, for instance the epidermis, dermis and/or subcutis, even more preferably the dermis, or for instance by applying a composition topically on the skin.

Preferably, the skin and/or skin layer(s) of the skin area, specifically the skin area [a], [b], [b₁] and/or [c], is immunological inactive and/or unchallenged, preferably at least partially, more preferably entirely immunological inactive and/or unchallenged. Furthermore, it is preferably spatially distanced to any site of immunological activity and/or challenge.

Preferably, the skin and/or skin layer(s) of the skin area, specifically the skin area [a], [b], [b₁] and/or [c], is located on the back, belly, buttocks, upper arm, forearm and/or upper leg more preferably the upper arm, forearm and/or upper leg.

The expression “immunological inactive and/or unchallenged skin” as mentioned in any of the embodiments described herein refers to skin and/or skin layer(s) of the skin area and/or the application area comprising, preferably consisting of a skin which is immunological inactive and/or unchallenged and, preferably spatially distanced to any site of immunological activity and/or challenge.

The expressions “immunological inactive and/or unchallenged” and/or “absence of an immune challenge and/or activity” as mentioned in any of the embodiments described herein may mean, preferably, means that any kind of active immune response is absent and/or any stimulus or inducer of the immune system is not present in an effective amount, that is an amount that is not sufficient to trigger an immune reaction, or is absent. Preferably an inflammation, an antigen, an autoantigen and/or an allergen like, for instance an inflamed joint, rheumatic joint, inflamed organ, allergic site, a wound and/or insect bite, regardless whether it is caused immunologically, autoimmunologically or does not have an immunological cause, is not present in an effective amount or to an effective extent or is absent or essentially absent, more preferably absent. Hence, preferably, “immunological inactive and/or unchallenged” as used in any of the embodiments described herein means that an inflammation, an antigen, an autoantigen and/or an allergen is not present in an effective amount or to an effective extent or preferably absent or essentially absent, more preferably absent.

The expression “site of immunological activity and/or challenge” as mentioned in any of the embodiments described herein may be, preferably, is any site, where an active immune response is present and/or any stimulus or inducer of the immune system is present in an amount sufficient to trigger an immune reaction. Preferably, a site of immunological activity and/or challenge is a site where an inflammation, an antigen, an autoantigen and/or an allergen is present like for instance an inflamed joint, rheumatic joint, inflamed organ, allergic site, a wound, an infection like a bacterial or viral infection, and/or insect bite and it may be, preferably, is caused immunologically or autoimmunologically.

According to the present invention, more preferably, the spatial distance of the skin area, specifically the skin area [a], [b], [b₁] and/or [c], to any site of immunological activity and/or challenge is independently as far as possible to any site of immunological activity and/or challenge. More preferably, the spatial distance is independently 0.5 cm or more, more preferably 1 cm or more, even more preferably 3 cm or more, still more preferably 5 cm or more, still even more preferably 10 cm or more. Usually, the skin area can only be located too close to any site of immunological activity and/or challenge but not too far away. Hence, there is no upper limit for the spatial distance. However, usually, the upper limit of the spatial distance is restricted by the subject's body expansion. Usually, the spatial distance is 3 meters or less, more preferably 2 meters or less, even more preferably 1.5 meters or less, still more preferably 1 meter or less, still even more preferably 50 cm or less.

The distance or spatial distance is preferably measured from a point on the outline of the skin area to a point on the outline of any site of immunological activity and/or challenge, wherein the points with the smallest distance to each other are taken. In case several sites of immunological activity and/or challenge are present, the skin area and/or skin area free of high amounts of antoantigen is preferably located spatially distanced to all these sites of immunological activity and/or challenge.

Most preferably, the skin and/or skin layer(s) of the skin area, specifically the skin area [a], [b], [b₁] and/or [c], are entirely immunological inactive and/or unchallenged and spatially distanced to any site of immunological activity and/or challenge as defined above.

The skin area, specifically the skin area [a], [b], [b₁] and/or [c], of the skin and/or skin layer(s), which are, preferably entirely, immunological inactive and/or unchallenged, may independently have any size sufficient to achieve the beneficial effects and may have a size of even about 1.5 m² or less. Preferably, the skin area is 1,000 cm² or less, more preferably 500 cm² or less, even more preferably 100 cm² or less, still more preferably 50 cm² or less, still even more preferably 25 cm² or less, further preferably 10 cm², even further preferably 5 cm² or less. Usually, the skin area is not smaller than 0.2 cm² or like 0.5 cm². Hence, a lower limit for the skin area is preferably 0.2 cm² or more, more preferably 0.5 cm² or more, even more preferably 1 cm² or more, still more preferably 2 cm² or more. Preferably, the skin area of the skin and/or skin layer(s), which are immunological inactive and/or unchallenged, is in the range of 1.5 m² or less and 0.2 cm² or more, more preferably 1,000 cm² or less and 0.2 cm² or more, even more preferably 500 cm² or less and 0.5 cm² or more, still more preferably 100 cm² or less and 0.5 cm² or more, still even more preferably 50 cm² or less and 1 cm² or more, further preferably 25 cm² or less and 1 cm² or more, even further preferably 10 cm² or less and 2 cm² or more. The skin area as mentioned in any of the embodiments is preferably measured as described in the Materials and Methods' section ‘Measurement of areas and distances relating to the skin’.

In an aspect of all embodiments of the invention, step (A), step (B), step (B₁) and/or step (C), may be, preferably are accomplished by topical application and/or by injection. Hence, preferably, in step (A), step (B), step (B₁) and/or step (C) the generating and/or administering is by topical application and/or by injection of the immunomodulatory substance(s) and/or skin-conditioning agent, preferably to or into the skin of an application area. Further details relating to step (A), step (B), step (B₁) and/or step (C) are described herein further below.

In case of an injection, in any of the embodiments of the present invention, the injection is preferably into a sub-topical layer of the skin, more preferably, into the epidermis, dermis and/or subcutis, even more preferably the dermis, of the skin and/or the injection is preferably placed 6 mm or less and 0.05 mm or more, more preferably 4 mm or less and 0.3 mm or more, even more preferably 3 mm or less and 0.5 mm or more in skin thickness direction into the skin, when measured from the skin surface, preferably when measured perpendicular to the skin surface towards the bones.

The expression “application area” as mentioned in any of the embodiments described herein typically refers to the area in whose associated skin the applying and/or injecting of the immunomodulatory substance(s) and/or skin-conditioning agent is accomplished to cause the effect of any of steps (A), step (B), step (B₁) and/or step (C) within or on the skin of the skin area, particularly in order to generate e.g. the increased concentration of the immunomodulatory substance(s), the accumulation of PBMCs, vasodilation, increased blood volume, increased sO₂, increased rHb, increased temperature and/or redness within or on the skin of the skin area or to administer the immunomodulatory substance(s) and/or skin-conditioning agent to the skin of the skin area.

Preferably, the application area is in case of step (A) an application area [a], in case of step (B) an application area [b], in case of step (B₁) an application area [b₁] and in case of step (A) an application area [c].

Preferably, the application area [a] is that of the skin area [a], the application area [b] is that of the skin area [b], the application area [b₁] is that of the skin area [b₁] and/or the application area [c] is that of the skin area [c].

Preferably, the application area [a] is arranged within and/or is congruent with the skin area [a], the application area [b] is arranged within and/or is congruent with the skin area [b], the application area [b₁] is arranged within and/or is congruent with the skin area [b₁] and/or the application area [c] is arranged within and/or is congruent with the skin area [c].

For instance, in step (B), the immunomodulatory substance(s) may be administered to the skin of the skin area [b] by applying the immunomodulatory substance(s) to the skin of the application area [b], e.g. by injecting the immunomodulatory substance(s) into the skin of the application area [b]. Or, for instance, in step (B), the immunomodulatory substance(s) may be administered to the skin of the skin area [b] by applying the immunomodulatory substance(s) topically to the skin of the application area [b].

For instance by applying a substance per topical application or injection to the skin of the application area, the concentration of such substance may be increased within the skin of the skin area. The substances may diffuse after for instance topical application into skin regions surrounding the skin of the application area. Hence, an increased concentration may be generated in a skin areal larger than the skin of the application area, designated herein with skin area. Similarly, for instance by applying conditioning energy like heat to the skin of the application area, the heat may radiate and distribute into adjacent skin regions surrounding the application area, thereby a for instance increased skin surface temperature will be generated within the skin of the skin area. Thereby, the temperature increase may not be restricted to the skin of the application area when applying the conditioning energy to the skin of the application area.

The expression “application area” as mentioned in any of the embodiments described herein refers to a two-dimensional expansion extending in parallel to the surface of the skin. In case of an injection, the application area may be, preferably, is the injection site. Preferably, the application area is congruent with the skin area and/or is arranged within the skin area, more preferably congruent. Hence, the application area may have a ring or planar ring of skin area surrounding the application area. Hence, an outline of the application area may be spaced apart to an outline of the skin area, wherein the outline of the skin area lies outside the application area, or, in other word, the outline of the application area lies within the skin area, thereby the skin area is larger than the application area. Hence, the skin area may be larger than the application area and the skin area totally arranged within the skin area.

The application is preferably indicated in m² (square meter(s)), cm² (square centimetre(s)), and/or mm² (square millimetre(s)). Reason for the ring or planar ring is, that the substances and/or the effects of a physical treatment, like heat, applied to the skin of the application area may diffuse and/or radiate into the adjacent skin regions (ring or planar ring) in a direction parallel to the skin surface and/or into the skin layer(s) (of course, a diffusion and/or radiation in skin thickness direction may also occur). Hence, the skin area in which skin the substances and/or effects of the physical treatment are still present in sufficient amounts or extent, may be larger (ring or planar ring) than the actual application area. Preferably, the spacing between the outline of the application area, specifically application area [a], [b], [b₁] and/or [c], and the skin area, specifically the skin area [a], [b], [b₁] and/or [c] respectively, is 3 cm or less, more preferably 2 cm or less, even more preferably 1.5 cm or less, still more preferably 1 cm or less, still even more preferably 0.5 cm or less. Usually, there is no lower limit for the spacing and it may become even 0 cm in case where the application area is congruent with the skin area. The spacing as mentioned in any of the embodiments is preferably measured as described in the Materials and Methods' section ‘Measurement of areas and distances relating to the skin’.

It shall be pointed out that the spacing may or may not vary over the entire ring or planar ring. Hence, the spacing is or is not necessarily constant for the entire ring, which may for instance depend on the constitution of the skin tissue surrounding the application area. Hence, the spacing may, preferably is, in the range of 0 cm or more and 3 cm or less, more preferably 0.1 cm or more and 2 cm or less, even more preferably 0.2 cm or more and 1.5 cm or less, still even more preferably 0.3 cm or more and 1 cm or less. In case of an injection, the ring or planar ring forms a disc or areal around the point injection site, wherein the radius of the disc or areal is the spacing and, preferably the areal is the skin area as defined in any of the embodiments described herein.

The application area, specifically application area [a], [b], [b₁] and/or [c], may have any two-dimensional shape as long as the beneficial effects can be achieved and may have a certain localisation on the subject's body. For instance, the application area may have a circular, triangular, rectangular, square, elliptic and/or polygonal shape, the shape may be, preferably, is irregular, lengthy and/or compact (the outline is rather short in respect of the encompassed application area, e.g. a square a shorter outline than a rectangle in respect of the encompassed area), it may have recesses, the application area can be divided into several individual areas and/or have an outline with concave and/or convex parts or any combination of these. Preferably, the application area is approximately circular, square or rather compact in shape and more preferably does not have recesses is divided into server individual areas.

The application area, specifically application area [a], [b], [b₁] and/or [c], may have independently any size sufficient to achieve the beneficial effects and may have even a size of 1.5 m² or less. Preferably, the application area is 1,000 cm² or less, more preferably 500 cm² or less, even more preferably 100 cm² or less, still more preferably 50 cm² or less, still even more preferably 25 cm² or less, further preferably 10 cm², even further preferably 5 cm² or less. Usually, the application area is not smaller than 0.01 mm² (for instance in case of an injection) or like 0.2 cm². Hence, a lower limit for the application area is preferably 0.01 mm² or more, more preferably 0.03 mm² or more, even more preferably 0.04 mm² or more, still more preferably 0.05 cm² or more, further preferably 0.2 cm² or more, even further preferably 0.5 cm² or more, still further preferably 1 cm² or more, still even further preferably 2 cm² or more. Preferably, the application area is in the range of 1.5 m² or less and 0.01 mm² or more, more preferably 1,000 cm² or less and 0.01 mm² or more, even more preferably 500 cm² or less and 0.01 mm² or more, still more preferably 100 cm² or less and 0.01 mm² or more, still even more preferably 50 cm² or less and 0.01 mm² or more, further preferably 25 cm² or less and 0.03 mm² or more, even further preferably 10 cm² or less and 0.04 mm² or more, still further preferably 5 cm² or less and 0.05 mm² or more. The application area as mentioned in any of the embodiments is preferably measured as described in the Materials and Methods' section ‘Measurement of areas and distances relating to the skin’.

In case of an injection, the application area, specifically application area [a], [b], [b₁] and/or [c], is independently in the range of preferably 15 mm² or less and 0.01 mm² or more, more preferably 5 mm² or less and 0.01 mm² or more, even more preferably 1 mm² or less and 0.01 mm² or more, still more preferably 0.8 mm² or less and 0.01 mm² or more, still even more preferably 0.2 mm² or less and 0.03 mm² or more and further preferably 0.1 mm² or less and 0.04 mm² or more,

In case of an administration by topical application, the application area, specifically application area [a], [b], [b₁] and/or [c], is independently in the range of preferably 1.5 m² or less and 0.01 cm² or more, more preferably 1,000 cm² or less and 0.01 cm² or more, even more preferably 500 cm² or less and 0.01 cm² or more, still more preferably 100 cm² or less and 0.02 cm² or more, still even more preferably 50 cm² or less and 0.2 cm² or more, further preferably 25 cm² or less and 0.5 cm² or more, even further preferably 10 cm² or less and 1 cm² or more, still further preferably 5 cm² or less and 2 cm² or more.

The expression “skin of a/the application area”, “skin layer(s) of a/the application area” or the like as mentioned in any of the embodiments described herein refers to the fragment of the skin or of one or more skin layers as defined above, covered and/or outlined by the application area. For instance, the expression ‘subcutis of the application area’ refers to the fragment of the subcutis covered and/or defined by the application area. For instance, the expression ‘dermis of the application area’ refers to the fragment of the dermis covered and/or defined by the application area.

Preferably, the skin and/or skin layer(s) of the application area, specifically application area [a], [b], [b₁] and/or [c], are immunological inactive and/or unchallenged, preferably at least partially, more preferably entirely immunological inactive and/or unchallenged and, preferably spatially distanced to any site of immunological activity and/or challenge.

Preferably, the skin and/or skin layer(s) of the application area, specifically application area [a], [b], [b₁] and/or [c], is located on the back, belly, buttocks, upper arm, forearm and/or upper leg more preferably the upper arm, forearm and/or upper leg.

More preferably, the spatial distance of the application area, specifically of application area [a], [b], [b₁] and/or [c], is independently as far as possible to any site of immunological activity and/or challenge. More preferably, the spatial distance is independently 0.5 cm or more, more preferably 1 cm or more, even more preferably 3 cm or more, still more preferably, 5 cm or more and still even more preferably 10 cm or more. Usually, the skin area can only be located to close to any site of immunological activity and/or challenge but not too far away. Hence, there is no upper limit for the spatial distance. However, usually, the upper limit of the spatial distance is restricted by the subject's body expansion. Usually, the spatial distance is 3 meters or less, more preferably 2 meters or less, even more preferably 1.5 meters or less, still more preferably 1 meters or less, still even more preferably 50 cm or less.

The distance or spatial distance is preferably measured from a point on the outline of the application area to a point on the outline of any site of immunological activity and/or challenge, wherein the points with the smallest distance to each other are taken. In case several sites of immunological activity and/or challenge are present, the application area and/or application area free of high amounts of antoantigen is preferably located spatially distanced to all these sites of immunological activity and/or challenge.

Most preferably, the skin and/or skin layer(s) of the application area, specifically application area [a], [b], [b₁] and/or [c], are entirely immunological inactive and/or unchallenged and spatially distanced to any site of immunological activity and/or challenge as defined above.

The application area, specifically application area [a], [b], [b₁] and/or [c], of the skin and/or skin layer(s), which are, preferably entirely, immunological inactive and/or unchallenged, may independently have any size sufficient to achieve the beneficial effects and may have a size of even about 1.5 m² or less. Preferably, such application area is 1,000 cm² or less, more preferably 500 cm² or less, even more preferably 100 cm² or less, still more preferably 50 cm² or less, still even more preferably 25 cm² or less, further preferably 10 cm², even further preferably 5 cm² or less. Usually, such application area is not smaller than 0.01 mm² (for instance in case of an injection) or like 0.5 cm² or 0.5 cm². Hence, a lower limit for such application area is preferably 0.01 mm² or more, more preferably 0.03 mm² or more, even more preferably 0.04 mm² or more, still even more preferably 0.05 cm² or more, further preferably 0.2 cm² or more, even further preferably 0.5 cm² or more, still further preferably 1 cm² or more, still even further preferably 2 cm² or more. Preferably, the application area, specifically application area [a], [b], [b₁] and/or [c], of the skin and/or skin layer(s), which are, preferably entirely, immunological inactive and/or unchallenged, is independently in the range of 1.5 m² or less and 0.01 mm² or more, more preferably 1,000 cm² or less and 0.01 mm² or more, even more preferably 500 cm² or less and 0.01 mm² or more, still more preferably 100 cm² or less and 0.01 mm² or more, still even more preferably 50 cm² or less and 0.01 mm² or more, further preferably 25 cm² or less and 0.03 mm² or more, even further preferably 10 cm² or less and 0.04 mm² or more, still further preferably 5 cm² or less and 0.05 mm² or more. Such application area as mentioned in any of the embodiments is preferably measured as described in the Materials and Methods' section ‘Measurement of areas and distances relating to the skin’.

In case of an injection, the application area, specifically application area [a], [b], [b₁] and/or [c], of the skin and/or skin layer(s), which are, preferably entirely, immunological inactive and/or unchallenged, is independently in the range of preferably 15 mm² or less and 0.01 mm² or more, more preferably 5 mm² or less and 0.01 mm² or more, even more preferably 1 mm² or less and 0.01 mm² or more, still more preferably 0.8 mm² or less and 0.01 mm² or more, still even more preferably 0.2 mm² or less and 0.03 mm² or more and further preferably 0.1 mm² or less and 0.04 mm² or more,

In case of an administration by topical application, the application area, specifically application area [a], [b], [b₁] and/or [c], of the skin and/or skin layer(s), which are, preferably entirely, immunological inactive and/or unchallenged, is independently in the range of preferably 1.5 m² or less and 0.01 cm² or more, more preferably 1,000 cm² or less and 0.01 cm² or more, even more preferably 500 cm² or less and 0.01 cm² or more, still more preferably 100 cm² or less and 0.02 cm² or more, still even more preferably 50 cm² or less and 0.2 cm² or more, further preferably 25 cm² or less and 0.5 cm² or more, even further preferably 10 cm² or less and 1 cm² or more, still further preferably 5 cm² or less and 2 cm² or more.

Preferably, in step (B) in any of the embodiments described herein, the immunomodulatory substance(s) is administered to the skin by topical application or by injection, more preferably by injection.

Preferably, in step (B) in any of the embodiments described herein, the immunomodulatory substance(s) is administered to the skin of the skin area.

More preferably, the immunomodulatory substance(s) is administered to the skin of the skin area and the increased concentration of the immunomodulatory substance(s) is generated within the skin of the skin area.

More preferably, the immunomodulatory substance(s) is administered to the skin of the skin area by applying the immunomodulatory substance(s) to the skin of the application area.

Even more preferably, the immunomodulatory substance(s) is administered to the skin of the skin area by applying the immunomodulatory substance(s) to the skin of the application area, wherein the increased concentration of the immunomodulatory substance(s) is generated within the skin of the skin area.

Preferably, in step (B) in any of the embodiments described herein, the immunomodulatory substance(s) is applied to the skin of the application area.

More preferably, the immunomodulatory substance(s) is applied to the skin of the application area, wherein the increased concentration of the immunomodulatory substance(s) is generated within the skin of the skin area. Preferably, in step (B) in any of the embodiments described herein, the immunomodulatory substance(s) is applied to the skin of the application area by topical application or by injection, more preferably by injection.

Preferably, in step (B) in any of the embodiments described herein, the application area is that of the respective skin area.

More preferably, the skin area is the skin area [b] and/or the application area is the application area [b].

Preferably, in step (B₁) in any of the embodiments described herein, the immunomodulatory substance(s) is administered to the skin by topical application or by injection, more preferably by injection.

Preferably, in step (B₁) in any of the embodiments described herein, the immunomodulatory substance(s) is administered to the skin of the skin area.

More preferably, the immunomodulatory substance(s) is administered to the skin of the skin area and the increased concentration of the immunomodulatory substance(s) is generated within the skin of the skin area.

More preferably, the immunomodulatory substance(s) is administered to the skin of the skin area by applying the immunomodulatory substance(s) to the skin of the application area.

Even more preferably, the immunomodulatory substance(s) is administered to the skin of the skin area by applying the immunomodulatory substance(s) to the skin of the application area, wherein the increased concentration of the immunomodulatory substance(s) is generated within the skin of the skin area.

Preferably, in step (B₁) in any of the embodiments described herein, the immunomodulatory substance(s) is applied to the skin of the application area.

More preferably, the immunomodulatory substance(s) is applied to the skin of the application area, wherein the increased concentration of the immunomodulatory substance(s) is generated within the skin of the skin area.

Preferably, in step (B₁) in any of the embodiments described herein, the immunomodulatory substance(s) is applied to the skin of the application area by topical application or by injection, more preferably by injection.

Preferably, in step (B₁) in any of the embodiments described herein, the application area is the application area of that respective skin area.

More preferably, the skin area is the skin area [b₁] and/or the application area is the application area [b₁].

Preferably, in step (C) in any of the embodiments described herein, the immunomodulatory substance(s) is administered to the skin by topical application or by injection, more preferably by topical application, more preferably by injection.

Preferably, in step (C) in any of the embodiments described herein, the immunomodulatory substance(s) is administered to the skin of the skin area.

More preferably, the immunomodulatory substance(s) is administered to the skin of the skin area and the increased concentration of the immunomodulatory substance(s) is generated within the skin of the skin area.

More preferably, the immunomodulatory substance(s) is administered to the skin of the skin area by applying the immunomodulatory substance(s) to the skin of the application area.

Even more preferably, the immunomodulatory substance(s) is administered to the skin of the skin area by applying the immunomodulatory substance(s) to the skin of the application area, wherein the increased concentration of the immunomodulatory substance(s) is generated within the skin of the skin area.

Preferably, in step (C) in any of the embodiments described herein, the immunomodulatory substance(s) is applied to the skin of the application area.

More preferably, the immunomodulatory substance(s) is applied to the skin of the application area, wherein the increased concentration of the immunomodulatory substance(s) is generated within the skin of the skin area. Preferably, in step (C) in any of the embodiments described herein, the immunomodulatory substance(s) is applied to the skin of the application area by topical application or by injection, more preferably by injection.

Preferably, in step (C) in any of the embodiments described herein, the application area is that of the respective skin area.

More preferably, the skin area is the skin area [c] and/or the application area is the application area [c].

Preferably, the skin area, particularly the skin area [a], [b], [b₁] and/or [c] is located on the back, belly, buttocks, upper arm, forearm and/or upper leg more preferably the upper arm, forearm and/or upper leg.

Preferably, the application area, particularly the application area [a], [b], [b₁] and/or [c] is located on the back, belly, buttocks, upper arm, forearm and/or upper leg more preferably the upper arm, forearm and/or upper leg.

If not mentioned otherwise, it is to be understood that any embodiment or definition described herein in terms of the ‘application area’ in general is independently and mutatis mutandis applicable to the application area [a], application area [b], application area [b₁] and/or application area [c], respectively, as mentioned in any of the embodiments described herein.

If not mentioned otherwise, it is to be understood that any embodiment or definition described herein in terms of the administration of the immunomodulatory substance(s), e.g. the cytokine(s), more preferably an interleukin(s), an interferon(s) and/or a neurotrophin(s), IFN-γ, IL-4, BDNF, IL-2 and/or skin-conditioning agent; the amounts, total amounts, amounts per kg body mass, total amounts per kg body mass, concentrations and/or administration doses administered thereof; the periods of time within which such amounts are administered; the frequency of administration; the combinations of such substances and agents administered; the way of administration; and/or whether administered successively, simultaneously, separately, combined together or any combination thereof; etc., are independently and mutatis mutandis applicable to the applying of the immunomodulatory substance(s) and/or skin-conditioning agent in any of the embodiments described herein.

As already stated above, in any of the embodiments described herein it is to be understood that it is preferred that the individual effects generated by steps (A), (B), (B₁) and/or (C) at least partially and/or totally overlap in time as well as spatially, for instance e.g. to be mutually dependent, supportive, interactive, complementary, additive, synergetic and/or to act by any other way together. Hence, it is to be understood that the method should be performed accordingly.

Hence, preferably, in any of the embodiments described herein the method is performed in such a way that the individual effects generated by steps (A), (B), (B₁) and/or (C) at least partially and/or totally overlap in time and/or spatially.

Preferably, in any of the embodiments described herein, the skin area [a] overlaps, totally overlaps and/or is congruent with one or more of skin area [b], skin area [b₁] and/or skin area [c].

More preferably, in any of the embodiments described herein, where applicable,
the skin areas [a] and [b];
the skin areas [a] and [c];
the skin areas [a], [b] and [c];
the skin areas [a] and [b₁];
the skin areas [a], [b₁] and [c]; and/or
the skin areas [a], [b], [b₁] and [c],
even more preferably
the skin areas [a], [b] and [c]; and/or
the skin areas [a], [b₁] and [c],
at least partially overlap, totally overlap and/or are congruent.

More preferably, the partial overlap(s), total overlap(s) and/or congruency/ies is/are of any of the skin areas which partially overlap, totally overlap and/or are congruent are enlarged or maximized.

Preferably, in any of the embodiments described herein, where applicable,
the skin areas [a] and [b] form overlapping skin area [a]/[b];
the skin areas [a] and [c] form overlapping skin area [a]/[c];
the skin areas [b] and [c] form overlapping skin area [b]/[c];
the skin areas [a], [b] and [c] form overlapping skin areas [a]/[b], [a]/[c] and/or [a]/[b]/[c], preferably overlapping skin areas [a]/[b] and/or [a]/[b]/[c], more preferably an overlapping skin area [a]/[b]/[c];
the skin areas [a] and [b₁] form an overlapping skin area [a]/[b₁];
the skin areas [b₁] and [c] form an overlapping skin area [b₁]/[c];
the skin areas [b] and [b₁] form an overlapping skin area [b]/[b₁];
the skin areas [a], [b₁] and [c] form overlapping skin areas [a]/[b₁], [a]/[c] and/or [a]/[b₁]/[c], preferably overlapping skin areas [a]/[b₁] and/or [a]/[b₁]/[c], more preferably an overlapping skin area [a]/[b₁]/[c]; and/or
the skin areas [a], [b], [b₁] and [c] form overlapping skin areas [a]/[b], [a]/[c], [a]/[b]/[c], [a]/[b₁], [b]/[b₁], [a]/[b₁]/[c] and/or [a]/[b]/[b₁]/[c], preferably [a]/[b], [a]/[b]/[c], [a]/[b₁], and/or [a]/[b₁]/[c], more preferably overlapping skin areas [a]/[b]/[c] and [a]/[b₁]/[c],
in case they at least partially overlap, totally overlap and/or are congruent.

More preferably, the overlapping skin area of any of the overlapping skin areas is enlarged or maximized.

It is explicitly mentioned that an partial overlap, total overlap and/or congruency of skin areas [b] and [c] and/or skin areas [b₁] and [c] without participation of skin area [a] or a formation of overlapping skin areas [b]/[c] and/or [b₁]/[c] without participation of skin area [a] is not excluded in any of the embodiments described herein. However, in any of the embodiments described herein, a participation of skin area [a] in any partial overlap, total overlap and/or congruency of the any skin areas [b], [b₁] and/or [c] and/or a participation of skin area [a] in the formation of any overlapping skin area is preferred.

Preferably, in any of the embodiments described herein:
the overlapping skin area [a]/[b];
the overlapping skin area [b]/[c];
the overlapping skin area [a]/[c];
the overlapping skin area [a]/[b]/[c];
the overlapping skin area [a]/[b₁];
the overlapping skin area [b₁]/[c];
the overlapping skin area [b]/[b₁];
the overlapping skin area [a]/[b₁]/[c]; and/or
the overlapping skin area [a]/[b]/[b₁]/[c],
may independently have any size sufficient to achieve the beneficial effects. More preferably, such skin area and/or the area sum of such area areas is/are enlarged or maximized. The skin area and/or the area sum and may have a size of even about 1 m² or less.

Preferably, the skin area is 1,000 cm² or less, more preferably 500 cm² or less, even more preferably 100 cm² or less, still more preferably 50 cm² or less, still even more preferably 25 cm² or less, further preferably 10 cm², even further preferably 5 cm² or less. Usually, the skin area is not smaller than 0.5 cm². Hence, a lower limit for the skin area is preferably 0.5 cm² or more, more preferably 1 cm² or more, still more preferably 2 cm² or more. The skin area and/or the area sum as mentioned in any of the embodiments is preferably measured as described in the Materials and Methods' section ‘Measurement of areas and distances relating to the skin’.

The expression “area sum” as mentioned in any of the embodiments described herein in respect to any skin area and/or application area typically refers to the total area of a certain type in case several skin areas, application areas, overlapping skin areas and/or overlapping application areas of that type are present. For instance, in case repetitions of some or all method steps are performed, several skin areas [a] may be generated or several overlapping skin areas [a]/[b] may be generated. The area sum of skin areas [a] is then the total area [a] after all of the several skin areas [a] have been added up. The area sum of the overlapping skin areas [a]/[b] is then the total overlapping skin area [a]/[b] after all of the several overlapping skin areas [a]/[b] have been added up.

The expression “partial overlap” or “partially overlapping” as mentioned in any of the embodiments described herein in respect to any skin area and/or application area typically means that two or more skin and/or application areas have an intersection in common and while other parts of the two or more skin areas and/or application areas do not overlap.

The expression “total overlap” or “totally overlapping” as mentioned in any of the embodiments described herein in respect to any skin area and/or application area typically means that at least one of two or more skin areas and/or application areas lies completely within one or more of the other skin areas and/or application areas. This may usually be the case if at least one or more skin areas and/or application areas is smaller than at least one or more other skin areas and/or application areas.

The expression “congruency” or “are/is congruent” as mentioned in any of the embodiments described herein in respect to any skin area and/or application area preferably defines a special case of total overlap and means that two or more skin and/or application areas are supposable. This usually in case one or more skin areas and/or application areas are identical in size and shape with at least one or more other skin areas and/or application areas.

The expression “overlapping skin area” as mentioned in any of the embodiments described herein typically refers to a skin area of partial overlap, total overlap and/or congruency of to two or more skin areas. Hence, the named skin areas have the overlapping skin area as intersection in common. Particularly “overlapping skin area [a]/[b]” refers to an area of partial overlap, total overlap and/or congruency of skin areas [a] and [b], “overlapping skin area [b]/[c]” refers to an area of partial overlap, total overlap and/or congruency of skin areas [b] and [c], “overlapping skin area [a]/[c]” refers to an area of partial overlap, total overlap and/or congruency of skin areas [a] and [c], “overlapping skin area [a]/[b]/[c]” refers to an area of partial overlap, total overlap and/or congruency of skin areas [a], [b] and [c], “overlapping skin area [a]/[b₁]” refers to an area of partial overlap, total overlap and/or congruency of skin areas [a] and [b₁], “overlapping skin area [b]/[c]” refers to an area of partial overlap, total overlap and/or congruency of skin areas [b₁] and [c], “overlapping skin area [b]/[b₁]” refers to an area of partial overlap, total overlap and/or congruency of skin areas [b] and [b₁], “overlapping skin area [a]/[b]/[c]” refers to an area of partial overlap, total overlap and/or congruency of skin areas [a], [b₁] and [c] and “overlapping skin area [a]/[b]/[b₁]/[c]” refers to an area of partial overlap, total overlap and/or congruency of skin areas [a], [b], [b₁] and [c], respectively.

If not mentioned otherwise, it is to be understood that any embodiment or definition described herein relating to ‘application area’, particularly, as regards the immunological inactivity and/or unchallenge, the special distance to any site of immunological activity and/or challenge and the size of the overlapping skin area, which is immunological inactive and/or unchallenged, is independently and mutatis mutandis applicable to the overlapping skin area [a]/[b], overlapping skin area [b]/[c], overlapping skin area [a]/[c], overlapping skin area [a]/[b]/[c], overlapping skin area [a]/[b₁], overlapping skin area [b₁]/[c], overlapping skin area [b]/[b₁], overlapping skin area [a]/[b₁]/[c], overlapping skin area [a]/[b]/[b₁]/[c], respectively, as mentioned in any of the embodiments described herein.

A site of inflammation may be, preferably, is any site where an inflammation is present, for instance an inflamed joint, inflamed organ, allergic site, a wound and/or insect bite and it may be, preferably, is immunologically or auto-immunologically. More preferably, the spatial distance of the overlapping skin area, particularly of any of the embodiments as described herein from the site of inflammation is 0.5 cm or more, more preferably 1 cm or more, even more preferably 3 cm or more, still more preferably, 5 cm or more and further preferably 10 cm or more. There is no upper limit for such distance, however, usually, it is 1.5 meters or less, more preferably 1 meters or less. The distance is preferably measured from a point on the outline of the overlapping skin area to a point on the outline of any site of inflammation, wherein the points with the smallest distance to each other are taken. In case several sites of inflammation are present, the overlapping skin area is preferably located spatially distanced to to all these sites of inflammation and free from inflammation or leasions.

Particularly, in the case that IL-4 and and BDNF are administered, it is preferred that the skin areas thereof do not overlap.

More preferably, particularly in case when in step (B) IL-4 and in step (B₁) BDNF is administered, the skin area [b] and the skin area [b₁] do not overlap and/or they are distanced apart or the overlap is minimized. Even more preferably the skin area [b], overlapping skin area [a]/[b] and/or overlapping skin area [a]/[b]/[c] do not overlap and/or they are distanced apart to skin area [b₁], overlapping skin area [a]/[b₁] and/or overlapping skin areas [a]/[b₁]/[c] and/or the overlap thereof is minimized.

More preferably, particularly in case when in step (B) IL-4 and in step (B₁) BDNF is administered, the method is performed in such a way that no overlapping skin area [b]/[b₁] is formed or the overlapping skin area [b]/[b₁] is minimized.

More preferably, particularly in case when in step (B) IL-4 and in step (B₁) BDNF is administered, the skin area [b], overlapping skin area [a]/[b] and/or overlapping skin area [a]/[b]/[c], preferably overlapping skin area [a]/[b]/[c], of the first set of steps and the skin area [b₁], overlapping skin area [a]/[b₁] and/or overlapping skin area [a]/[b₁]/[c], preferably overlapping skin area [a]/[b₁]/[c], of the second set of steps do not overlap and/or they are distanced apart or the overlap is minimized.

The application areas [a], [b], [b₁] and/or [c] in any of the embodiments described herein independently may or may not partially overlap, totally overlap and/or may or may not be congruent and may be even distanced apart. Preferably, the distance for any, preferably all, of the distanced apart applications areas to each other is 1 cm or less, more preferably 0.5 cm or less.

The distance is preferably measured from a point on the outline of any of such application areas to a point on the outline of any of the other distanced apart application areas, wherein the points with the smallest distance to each other are taken. Preferably, the application areas [a], [b], [b₁] and/or [c] partially overlap, totally overlap and/or are congruent, wherein more preferably all embodiments described herein for skin areas [a], [b], [b₁] and/or [c] and overlapping skin areas [a]/[b], [a]/[c], [c]/[b], [a]/[b]/[c], [a]/[b₁], [c]/[b₁], [a]/[b₁]/[c] and/or [a]/[b]/[b₁]/[c] apply mutatis mutandis to application areas [a], [b], [b₁] and/or [c] and overlapping application areas [a]/[b], [a]/[c], [c]/[b], [a]/[b]/[c], [a]/[b₁], [c]/[b₁], [a]/[b₁]/[c] and/or [a]/[b]/[b₁]/[c].

More preferably, particularly in case when in step (B) IL-4 and in step (B₁) BDNF is administered, the application area [b] and the application area [b₁] do not overlap and/or they are distanced apart or the overlap is minimized. Even more preferably the application area [b], overlapping application area [a]/[b] and/or overlapping application area [a]/[b]/[c] do not overlap and/or they are distanced apart to application area [b₁], overlapping application area [a]/[b₁] and/or overlapping application areas [a]/[b₁]/[c] and/or the overlap thereof is minimized.

More preferably, particularly in case when in step (B) IL-4 and in step (B₁) BDNF is administered, the method is performed in such a way that no overlapping application area [b]/[b₁] is formed or the overlapping application area [b]/[b₁] is minimized.

More preferably, particularly in case when in step (B) IL-4 and in step (B₁) BDNF is administered, the application area [b], overlapping application area [a]/[b] and/or overlapping application area [a]/[b]/[c], preferably overlapping application area [a]/[b]/[c], of the first set of steps and the application area [b₁], overlapping application area [a]/[b₁] and/or overlapping application area [a]/[b₁]/[c], preferably overlapping application area [a]/[b₁]/[c], of the second set of steps do not overlap and/or they are distanced apart or the overlap is minimized.

Preferably, particularly in case when in step (B) IL-4 and in step (B₁) BDNF is administered, the distance of such distanced apart skin areas, overlapping skin areas, application areas and/or overlapping application areas is 0.5 cm or more, more preferably 1 cm or more, even more preferably 3 cm or more, still more preferably, 5 cm or more and still even more preferably 10 cm or more. There is no upper limit for such distance, however, usually, it is 3 meters or less, more preferably 1.5 meters or less. The distance is preferably measured from a point on the outline of the skin area or overlapping skin area, respectively, to a point on the outline of the skin area or overlapping skin area, to which it is to be distanced apart, wherein the points with the smallest distance to each other are taken. There is no upper limit for the distance. However, usually, the upper limit of the distance is restricted by the subject's body expansion. Usually, the spatial distance is 4 meters or less, more preferably 3 meters or less, even more preferably 2 meters or less, still more preferably 1.5 meters or less. Even more preferably, the distance is in the range of 0.5 cm or more and 4 meters or less, more preferably 1 cm or more and 3 meters or less, even more preferably 3 cm or more and 2 meters or less, still more preferably 5 cm or more and 1.5 meters or less, still even preferably 10 cm or more and 1.5 meters or less.

The immunomodulatory substance(s) in step (B), in step (B₁) and/or in step (C) as mentioned in any of the embodiments described herein may be administered and/or applied by any suitable route of administration or application to the skin. Preferably, the immunomodulatory substance(s) is administered by topical application and/or by injection. Preferably, in any of the embodiments described herein, the immunomodulatory substance(s) is prepared for and formulated in a way suitable for an administration by topical application or for an injection, for example as described herein. More preferably, it is in the form of any of the pharmaceutical compositions, the injectable dosage forms, the topical dosage forms, medical devices and/or the kits of parts according to the present invention or as mentioned in any of the embodiments described herein suitable for the administration by topical application and/or injection.

A topical application preferably is a non-invasive kind of administration. Preferably, a topical application includes every administration that is applied to a particular topical place on or in the body, for example an application to any surface of the body e.g. the skin. Administrations by topical application include epicutaneous application, meaning that e.g. the immunomodulatory substance(s) and/or skin-conditioning agent, is applied directly to the skin. For administrations by topical application any form of a formulation may be used for instance selected from an ointment, emulsion, solution, suspension, paste, gel, lotion, tincture, particulate material, powder, liquid or solid preparations for application to the skin, slow-release formulation, wadding, tamponade, crème, balm, foam, gel, spray, stick, liquid plaster, spray plaster, plaster for intradermal administration, time-release plaster, transdermal patch, plaster, pad, wadding, padding, dressing, compress, bandage, optionally of multi-compartment type as mentioned in any of the embodiments described herein, a dermatological preparation, preferably in the form of an application for external use. For the administrations by topical application in any of the embodiments described herein, preferably any of the pharmaceutical compositions, topical dosage forms, medical devices and/or kits of parts as mentioned in any of the embodiments described herein is used, which is suitable for an administration by topical application.

Preferably, the topical application in any of the embodiments described herein is placed on the back, belly, buttocks, upper arm, forearm and/or upper leg more preferably the upper arm, forearm and/or upper leg.

An injection is preferably an invasive kind of administration. Preferably, an injection, in any of the embodiments described herein, is preferably into a sub-topical layer, more preferably, into the epidermis, dermis and/or subcutis, even more preferably the dermis, and/or the injection is preferably placed 6 mm or less and 0.05 mm or more, more preferably 4 mm or less and 0.3 mm or more, even more preferably 3 mm or less and 0.5 mm or more in skin thickness direction, preferably when measured from the skin surface towards the bones. Preferably, for the injection in any of the embodiments described herein any of the pharmaceutical compositions, injectable dosage form and/or kits of parts according to the present invention or as mentioned in any of the embodiments described herein, which are suitable for injection, is used for the injection. For administrations by injection any form of a formulation may be used for instance selected from an ointment, emulsion, solution, suspension, paste, gel, lotion, tincture, particulate material, liquid preparations for injection, slow-release formulation, crème, balm, foam, gel, a dermatological preparation, preferably in the form of an application for injection, cartridge for an injection pen or the medical device as described herein, carpule, vial, ampule, prefilled injection pen, prefilled syringe, prefilled multichannel syringe, a medical device, optionally of a multichannel and/or multi-compartment type as mentioned in any of the embodiments described herein. For the administrations by injection in any of the embodiments described herein, preferably any of the pharmaceutical compositions, injectable dosage forms and/or kits of parts as mentioned in any of the embodiments described herein is used, which is suitable for an administration by injection.

Preferably, the injection in any of the embodiments described herein is placed into the back, belly, buttocks, upper arm, forearm and/or upper leg more preferably the upper arm, forearm and/or upper leg.

Preferably, in any of the embodiments described herein the administering of the immunomodulatory substance(s) in step (B), in step (B₁) and/or in step (C) to the skin may be, preferably, is accomplished by topical application and/or by injection, more preferably by injection.

More preferably, in any of the embodiments described herein the administering of the IFN-γ and/or IFN-γ-like acting substance(s) to the skin may be, preferably, is accomplished by topical application and/or by injection.

More preferably, in any of the embodiments described herein the administering of the IL-4 and/or IL-4-like acting substance(s) to the skin may be, preferably, is accomplished by topical application and/or by injection.

More preferably, in any of the embodiments described herein the administering of the BDNF and/or BDNF-like acting substance(s) to the skin may be, preferably, is accomplished by topical application and/or by injection. More preferably, in any of the embodiments described herein the administering of the IL-2 and/or IL-2-like acting substance(s) to the skin may be, preferably, is accomplished by topical application and/or by injection.

Preferably, in any of the embodiments described herein the administering of the immunomodulatory substance(s) in step (B), step (B₁) and/or step (C) may be carried out by administering the immunomodulatory substance(s) as such or as an active ingredient of a composition, dosage form and/or kits of parts, i.e. as a composition, dosage form and/or kit of parts comprising the immunomodulatory substance(s).

Preferably, the composition, dosage form and/or kits of parts is any of the pharmaceutical compositions, topical dosage forms and/or kits of parts according to the present invention or as mentioned in any of the embodiments described herein. This applies generally in the present invention and hence applies to any of the embodiments of the invention described herein.

Furthermore, in any of the embodiments described herein the administering of the immunomodulatory substance(s) in step (B), step (B₁) and/or step (C) may involve to administer the immunomodulatory substance(s): in the form of any of the pharmaceutical compositions comprising the immunomodulatory substance(s); in a topical dosage form comprising the immunomodulatory substance(s); in an injectable dosage form comprising the immunomodulatory substance(s); and/or in the form of a kits of parts comprising the immunomodulatory substance(s).

Preferably, the compositions is any of the pharmaceutical compositions according to the present invention or as mentioned in any of the embodiments described herein. Preferably, the topical dosage form is the topical dosage form according to the present invention or as mentioned in any of the embodiments described herein. Preferably, the injectable dosage form is the injectable dosage form according to the present invention or as mentioned in any of the embodiments described herein. Preferably, the kits of parts is any of the kits of parts according to the present invention or as mentioned in any of the embodiments described herein. This also applies generally in the present invention and hence applies to any of the embodiments of the invention described herein.

Without wishing to be bound to theory, it is believed that regulatory T-cells and/or helper T-cells, or a subset thereof, are considered to be the effectors of the present invention. Hence, generating a larger amount of regulatory T-cells and/or helper T-cells, or a subset thereof, can lead to an improved or stronger beneficial effect. For instance, inflammatory parameters may be decreased to a greater extent, swelling of joints is further reduced and larger inflammatory lesions caused by the inflammatory disease, immunological disease and/or autoimmunological diseases may become inflammatory inactive. Within skin having a larger skin area more PMBCs may be recruited, accumulated and finally affected. Similarly, within skin having a larger skin area more LHs and peripheral T_{reg}s may be affected. Therefore, it may be an aim that for instance an overlapping skin area [a]/[b] and/or [a]/[b]/[c], preferably the overlapping skin area [a]/[b]/[c] has an enlarged or even maximized size to generate an sufficient amount of affected cells.

Therefore, in order to increase the beneficial effects, the overlapping skin areas may be enlarged or even maximized. This may for instance be achieved by adjusting or increasing the amount of the immunomodulatory substance(s) like IFN-γ, IL-4, BDNF and/or IL-2 within the limits of the embodiments of the present invention described herein. A larger amount may diffuse and/or spreads a longer distance into the adjacent skin regions (ring or planar ring) in a direction parallel to the skin surface and/or into the skin layer(s) than a lower concentration. Thereby a larger skin area is produced to which a sufficient concentration of the immunomodulatory substance(s) is administered and in which a larger amount affected LHs, affected peripheral T_{reg}s and/or affected PBMCs can be generated. However, the spreading may occur not only parallel to the skin surface but also in the direction perpendicular to the skin's surface into the tissues underneath the skin. Without wishing to be bound to theory, it is believed that in the tissues lying underneath the skin no beneficial effect is achieved and an unnecessary high amount of the immunomodulatory substance(s) is administered. It should be a general aim to administer the lowest possible amount of a drug to a patient. Additionally, it is a preferred aspect of the present invention to administer the lowest possible amount of the immunomodulatory substance(s) in order to avoid a systemic increase thereof. Thereby, as already explained above, it is intended to avoid the generation of an increased concentration of the immunomodulatory substance(s), for instance at sites of inflammation, like inflamed joints. For instance naïve T-cells may mature in the presence of IFN-γ and high amounts of antigen/autoantigen/allergen towards cytotoxic T-cells, which may act pro-inflammatory, thereby the may decrease, cancel or even reverses the beneficial effects achieved by the present invention, particularly, the anti-inflammatory effect of the regulatory T-cells and/or helper T-cells, or the generated subset thereof, which are believed to be generated by the present invention.

The overlapping skin area can also be enlarged or maximized by multiply performing or performing several or multiple times the entire method or some steps thereof multifocally that is at different locations distanced apart from each other on the subject's skin, wherein each time a comparatively small amount of the immunomodulatory substance(s) is administered.

Without wishing to be bound to theory, it is believed that by a multifocal performance of the method or particular steps thereof (particularly steps (B), (B₁) and/or (C)), the spreading and diffusing of the immunomodulatory substance(s) into the tissues underneath the skin may be reduced while at the same time the size of the overlapping skin area can be enlarged or maximized. Consequently, with the same or even a reduced amount of the immunomodulatory substance(s), skin of a larger skin area can be provided with for instance immunomodulatory substance(s) while in the sum less tissue volume underneath the skin receives the immunomodulatory substance(s). For this purpose, for instance the entire method comprising steps (A), (B) and (C) may be performed multiple times. Alternatively, e.g. steps (B) and/or (C) may be performed multiple times, wherein step (A) is performed only once. This may for instance be accomplished when a heat pad or heat crème is used. The resulting skin area [a] of the skin affected by performing step (A) is then usually of sufficient size (for instance about 4 cm×6 cm) in order to inject into there at several locations and distanced apart from each other the immunomodulatory substance(s) of step (B) and of step (C). This aspect of reducing the exposure of the deeper layers underneath the skin to the immunomodulatory sibstance(s) is not only of interest to further reduce the needed effective amount of substances, but may also be beneficial for certain diseases, such as Bechterew's disease. It is believed, without wishing to be bound thereto, that the distance to the antigen typical for such disease (like muscles and connective tissue) is maximized and the exposure of these critical tissues to effective amounts of the immunomodulatory substance(s) can be minimized or prevented (for further details see also Inventive Examples 11 and 12 and the considerations detailed herein regarding the avoidance of generating pro-inflammatory cytotoxic T-cells when naïve T-cells are exposed to their antigen).

Furthermore, without wishing to be bound to theory, it is believed that when step (A) is performed multiple times, i.e. step (A) is for instance repeated, or it is performed for a proponged time or the effect of step (A) (that is the generating of the accumulation of PBMCs, of the accumulation of autoantigen, vasodilation, increased blood volume, increased sO₂, increased rHb, increased temperature and/or redness within/on the skin) is maintained for a prolonged time and more PBMCs can be recruited into the skin. Thereby, more PBMCs may be brought in contact with the immunomodulatory substance(s) to develop into e.g. regulatory T-cells, and/or for instance with dendritic cells residing within the skin. Furthermore, autoantigen may be accumulated within the skin for a longer period of time. Moreover, LHs may be exposed for a longer period of time to the autoantigen and e.g. increased temperatures, which are believed to lead to a better stabilisation of the MCH-autoantigen complex by heat shock protein (chaperone) expression. It is noted that the dendritic cells may be also conditioned by the immunomodulatory substance(s) to influence and affect the PBMCs to develop into e.g. regulatory T-cells. Consequently, by performing step (A) multiple times it is believed that more effector cells like helper T-cells, or a subset thereof, and regulatory T-cells may be generated leading to an amplification of the beneficial effect (for further details see also Inventive Examples 10).

Hence, preferably, in any of the embodiments described herein, the method is performed in a way that, where applicable, the overlapping skin area [a]/[b], [a]/[c], [a]/[b]/[c], [a]/[b₁], [a]/[b₁]/[c] and/or [a]/[b]/[b₁]/[c], more preferably the area sum of the overlapping skin areas [a]/[b], [a]/[c], [a]/[b]/[c], [a]/[b₁] and/or [a]/[b₁]/[c] is/are enlarged or maximized.

Preferably, in any of the embodiments described herein comprising steps (A) and (B), the method is performed in a way that the overlapping skin area [a]/[b] is/are enlarged or maximized.

Preferably, in any of the embodiments described herein comprising steps (A) and (C), the method is performed in a way that the overlapping skin area [a]/[c] is/are enlarged or maximized.

Preferably, in any of the embodiments described herein comprising steps (A), (B) and (C), the method is performed in a way that the overlapping skin areas [a]/[b], [a]/[c] and/or [a]/[b]/[c], more preferably the overlapping skin areas [a]/[b] and/or [a]/[b]/[c], even more preferably the overlapping skin area [a]/[b]/[c] is/are enlarged or maximized.

Preferably, in any of the embodiments described herein comprising steps (A), (B), (B₁) and (C) the method is performed in a way that the overlapping skin areas [a]/[b], [a]/[c], [a]/[b]/[c], [a]/[b₁], [a]/[b₁]/[c] and/or [a]/[b]/[b₁]/[c], more preferably skin areas [a]/[b], [a]/[c], [a]/[b]/[c], [a]/[b₁] and/or [a]/[b₁]/[c], even more preferably skin areas [a]/[b], [a]/[b]/[c], [a]/[b₁] and/or [a]/[b₁]/[c], still even more preferably skin areas [a]/[b]/[c] and/or [a]/[b₁]/[c] is/are enlarged or maximized.

Preferably, in any of the embodiments described herein, the method is performed in a way that, where applicable, the area sum of the overlapping skin areas [a]/[b], [a]/[c], [a]/[b]/[c], [a]/[b₁], [a]/[b₁]/[c] and/or [a]/[b]/[b₁]/[c], more preferably the area sum of the overlapping skin areas [a]/[b], [a]/[c], [a]/[b]/[c], [a]/[b₁] and/or [a]/[b₁]/[c], even more preferably the area sum of the overlapping skin areas [a]/[b], [a]/[b]/[c], [a]/[b₁] and/or [a]/[b₁]/[c], still more preferably the area sum of the overlapping skin areas [a]/[b]/[c] and/or [a]/[b₁]/[c] is/are enlarged or maximized.

More preferably, in any of the embodiments described herein, step (A), step (B), step (B₁) and/or step (C) are performed multiple times in such a way that the area sum of the overlapping skin areas [a]/[b], [a]/[c], [a]/[b]/[c], [a]/[b₁], [a]/[b₁]/[c] and/or [a]/[b]/[b₁]/[c], even more preferably the area sum of the overlapping skin areas [a]/[b], [a]/[c], [a]/[b]/[c], [a]/[b₁] and/or [a]/[b₁]/[c], still more preferably the area sum of the overlapping skin areas [a]/[b], [a]/[b]/[c], [a]/[b₁] and/or [a]/[b₁]/[c], still even more preferably the area sum of the overlapping skin areas [a]/[b]/[c] and/or [a]/[b₁]/[c] is/are enlarged or maximized in comparison to the area sum of the method performed without any multiple performances.

Preferably, the number of the multiple performances for step (A), step (B), step (B₁) and/or step (C) is independently 2 to 3000 times, more preferably 2 to 50 times, even more preferably 2 to 10 times, still more preferably 2 to 5 times, still even more preferably 1 to times, further preferably 1 or 2 times.

The expression “performed multiple times” is also referred to as in noun-form as “multiple performances”.

In a more preferred aspect of all embodiments of the present invention described herein, the present invention relates to an immunomodulatory substance(s) and/or a skin-conditioning agent for use in a method for treating and/or preventing an inflammatory disease, immunological disease and/or autoimmunological disease in a subject, wherein the method comprises, preferably consists of, the steps of:
(A) which is selected from one or more of steps (A-0), (A-1), (A-2), (A-3), (A-4), (A-5), (A-6), (A-7) and/or (A-8); and
(B) administering the immunomodulatory substance(s) to the skin of the subject,
wherein step (A) and/or step (B) is performed multiple times,
and to said method as such.

Preferably, step (A) and/or step (B) is performed 2 to 3000 times. The step may for instance be easily performed 3000 times when using for instance a microneedle patch having 3000 microneedles.

More preferably, step (A) is performed 2 to 50 times, even more preferably 2 to 10 times, still more preferably 2 to 5 times.

More preferably, step (B) is performed 2 to 3000 times, even more preferably 2 to 1000 times, still more preferably 2 to 10 times, still even more preferably 2 to 5 times.

More preferably, step (A) and step (B) are performed 2 to 3000 times, even more preferably 2 to 1000 times, still more preferably 2 to times, still even more preferably 2 to 5 times.

Preferably, in step (A) the skin is of a skin area [a] and in step (B) the skin is of a skin area [b].

More preferably, the multiple performances are carried out in such a way that the area sum of the overlapping skin areas [a]/[b] resulting from the method comprising multiple performances, is enlarged or maximized in comparison to the area sum of the method, which does not comprise any of the multiple performances.

More preferably, at least one of the overlapping skin area [a]/[b] resulting from the method comprising multiple performances only partially overlaps and/or does not overlap with at least one other overlapping skin area [a]/[b] resulting from the method comprising multiple performances.

Preferably, step (A), step (B) and any or all of the multiple performances as mentioned in any of the embodiments described herein may be performed in any order, separately, successively, combined together, simultaneously or any combination thereof.

Furthermore, each of step (A), step (B) and the multiple performances as mentioned in any of the embodiments described herein may independently be performed successively, simultaneously, separately, combined together or any combination thereof of one or more of the other step(s) (A), step(s) (B) and the multiple performances.

Preferably, the embodiments relating to whether any of step (A), step (B), step (B₁) and/or step (C) may or may not be completed before the next step is performed apply mutatis mutandis also to step (A), step (B) and the multiple performances as mentioned in any of the embodiments described herein.

If not mentioned otherwise, it is to be understood that any embodiment of the present invention described herein:
for the immunomodulatory substance(s) for use according to the present invention;
for the inflammatory disease, immunological disease and/or autoimmunological disease;
for the immunomodulatory substance(s) in step (B); and/or
for steps (A) and/or (B),
is independently and mutatis mutandis applicable to the more preferred aspect of all embodiments of the present invention comprising, that is the above aspect of the present invention which comprises the multiple performances. Thereby it is to be understood that each multiple performance of steps (A) and/or (B) may be independently performed according to any of the embodiments as described herein. Hence, for instance, a repetition of step (A) may or may not be performed the same way as another repetition of step (A), for instance in respect of the used agent (e.g. skin-conditioning agent), the concentration, the size of the skin area etc. Same applies to steps (B), for instance in respect of the immunomodulatory substance(s), their concentrations, whether administered by topical application or by injection, the size of the skin area etc. For example, in one repetition of step (B), for instance, an amount of 100 IU of the immunomodulatory substance may be administered while in another repetition thereof 200 IU are administered. Preferably, the repetitions of steps (A) and/or (B) are performed the same.

Alternatively, in a more preferred aspect of all embodiments of the present invention described herein, the present invention relates to an immunomodulatory substance(s), more preferably to IL-2, and/or a skin-conditioning agent for use in a method for treating and/or preventing an inflammatory disease, immunological disease and/or autoimmunological disease in a subject, wherein the method comprises, preferably consists of, the steps of:
(A) which is selected from one or more of steps (A-0), (A-1), (A-2), (A-3), (A-4), (A-5), (A-6), (A-7) and/or (A-8); and
(C) administering the immunomodulatory substance(s), more preferably IL-2, to the skin of the subject, wherein step (A) and/or step (C) is performed multiple times,
and to said method as such.

Preferably, step (A) and/or step (C) is performed 2 to 3000 times.

More preferably, step (A) is performed 2 to 50 times, even more preferably 2 to 10 times, still more preferably 2 to 5 times.

More preferably, step (C) is performed 2 to 3000 times, even more preferably 2 to 1000 times, still more preferably 2 to 10 times, still even more preferably 2 to 5 times.

More preferably, step (A) and step (C) are performed 2 to 3000 times, even more preferably 2 to 1000 times, still more preferably 2 to times, still even more preferably 2 to 5 times.

Preferably, in step (A) the skin is of a skin area [a] and in step (C) the skin is of a skin area [c].

More preferably, the multiple performances) are carried out in such a way that the area sum of the overlapping skin areas [a]/[c] resulting from the method comprising multiple performances, is enlarged or maximized in comparison to the area sum of the method, which does not comprise any of the multiple performances.

More preferably, at least one of the overlapping skin area [a]/[c] resulting from the method comprising multiple performances only partially overlaps and/or does not overlap with at least one other overlapping skin area [a]/[c] resulting from the method comprising multiple performances.

Preferably, step (A), step (C) and any or all of the multiple performances as mentioned in any of the embodiments described herein may be performed in any order, separately, successively, combined together, simultaneously or any combination thereof.

Furthermore, each of step (A), step (C) and any or all of the multiple performances as mentioned in any of the embodiments described herein may independently be performed successively, simultaneously, separately, combined together or any combination thereof in respect of the one or more of the other step(s) (A), step(s) (C) and the multiple performances.

Preferably, the embodiments relating to whether any of step (A), step (B), step (B₁) and/or step (C) may or may not be completed before the next step is performed apply mutatis mutandis also to step (A), step (B), and the multiple performances as mentioned in any of the embodiments described herein.

If not mentioned otherwise, it is to be understood that any embodiment of the present invention described herein:
for the immunomodulatory substance(s) for use according to the present invention;
for the inflammatory disease, immunological disease and/or autoimmunological disease;
for the immunomodulatory substance(s) in step (C); and/or
for steps (A) and/or (C),
is independently and mutatis mutandis applicable to the still even more preferred aspect of all embodiments of the present invention comprising, that is the above aspect of the present invention which comprises the multiple performances. Thereby it is to be understood that each multiple performances of steps (A) and/or (C) in repetition steps may be independently performed according to any of the embodiments as described herein. Hence, for instance, a repetition of step (A) may or may not be performed the same way as another repetition of step (A), for instance in respect of the used agent (e.g. skin-conditioning agent), the concentration, the size of the skin area etc. Same applies to step (C), for instance in respect of the immunomodulatory substance(s), their concentrations, whether administered by topical application or by injection, the size of the skin area etc. Preferably, the repetitions of steps (A) and/or (C) are performed the same.

In an even more preferred aspect of all embodiments of the present invention described herein, the present invention relates to an immunomodulatory substance(s) and/or a skin-conditioning agent for use in a method for treating and/or preventing an inflammatory disease, immunological disease and/or autoimmunological disease in a subject, wherein the method comprises, preferably consists of, the steps of:
(A) which is selected from one or more of steps (A-0), (A-1), (A-2), (A-3), (A-4), (A-5), (A-6), (A-7) and/or (A-8); and
(B) administering the immunomodulatory substance(s) to the skin of the subject; and
(C) administering the immunomodulatory substance(s) to the skin of the subject,
wherein the immunomodulatory substance(s) administered in step (C) is different from the immunomodulatory substance(s) administered in step (B),
wherein step (A), step (B) and/or step (C) is performed multiple times,
and to said method as such.

Preferably, step (A), step (B) and/or step (C) is performed 2 to 3000 times.

More preferably, step (A) is performed 2 to 50 times, even more preferably 2 to 10 times, still more preferably 2 to 5 times.

More preferably, step (B) is performed 2 to 3000 times, even more preferably 2 to 1000 times, still more preferably 2 to 10 times, still even more preferably 2 to 5 times.

More preferably, step (C) is performed 2 to 3000 times, even more preferably 2 to 1000 times, still more preferably 2 to 10 times, still even more preferably 2 to 5 times.

More preferably, step (A), step (B) and step (C) are performed 2 to 3000 times, even more preferably 2 to 1000 times, still more preferably 2 to 10 times, still even more preferably 2 to 5 times.

Even more preferably, step (A) is performed 2 to 50 times, still more preferably 2 to 10 times, still even more preferably 2 to 5 times and steps (B) and (C) are performed 2 to 3000 times, still more preferably 2 to 1000 times, still even more preferably 2 to 10 times, further preferably 2 to 5 times.

Preferably, in step (A) the skin is of a skin area [a], in step (B) the skin is of a skin area [b] and in step (C) the skin is of a skin area [c].

More preferably, the multiple performances are carried out in such a way that the area sum of the overlapping skin areas [a]/[b], [a]/[c] and/or [a]/[b]/[c], even more preferably the overlapping skin areas [a]/[b] and/or [a]/[b]/[c], still more preferably the overlapping skin areas[a]/[b]/[c], resulting from the method comprising multiple performances, is enlarged or maximized in comparison to the area sum of the method, which does not comprise any of the multiple performances.

More preferably, at least one of the overlapping skin areas [a]/[b] and/or [a]/[b]/[c] resulting from the method comprising multiple performances only partially overlaps and/or does not overlap with at least one other of the overlapping skin area(s) [a]/[b] and/or [a]/[b]/[c] resulting from the method comprising multiple performances.

Preferably, step (A), step (B), step (C) and any or all of the multiple performances as mentioned in any of the embodiments described herein may be performed in any order, separately, successively, combined together, simultaneously or any combination thereof.

Furthermore, each of step (A), step (B), step (C) and any of the multiple performances as mentioned in any of the embodiments described herein may independently be performed successively, simultaneously, separately, combined together or any combination thereof in respect of the one or more of the other step(s) (A), step(s) (B), step(s) (C) and multiple performances.

Preferably, the embodiments relating to whether any of step (A), step (B), step (B₁) and/or step (C) may or may not be completed before the next step is performed apply mutatis mutandis also to step (A), step (B), step (C) and the multiple performances as mentioned in any of the embodiments described herein.

If not mentioned otherwise, it is to be understood that any embodiment of the present invention described herein:
for the immunomodulatory substance(s) for use according to the present invention;
for the inflammatory disease, immunological disease and/or autoimmunological disease;
for the immunomodulatory substance(s) in step (B) and/or (C); and/or
for steps (A), (B) and/or (C),
is independently and mutatis mutandis applicable to the still even more preferred aspect of all embodiments of the present invention comprising, that is the above aspect of the present invention which comprises the multiple performances. Thereby it is to be understood that each multiple performance of steps (A), (B) and/or (C) may be independently performed according to any of the embodiments as described herein. Hence, for instance, a repetition of step (A) may or may not be performed the same way as another repetition of step (A), for instance in respect of the used agent (e.g. skin-conditioning agent), the concentration, the size of the skin area etc. Same applies to steps (B) and/or (C), for instance in respect of the immunomodulatory substance(s), their concentrations, whether administered by topical application or by injection, the size of the skin area etc. Preferably, the repetitions of steps (A), (B) and/or (C) are performed the same.

In another even more preferred aspect of all embodiments of the present invention described herein, the present invention relates to an immunomodulatory substances and/or a skin-conditioning agent for use in a method for treating and/or preventing an inflammatory disease, immunological disease and/or autoimmunological disease in a subject, wherein step (B) is performed a second time as (B₁), wherein the method comprises, preferably consists of, the steps of:
(A) which is selected from one or more of steps (A-0), (A-1), (A-2), (A-3), (A-4), (A-5), (A-6), (A-7) and/or (A-8); and
(B) administering the immunomodulatory substance(s) to the skin of said subject; and
(B₁) administering the immunomodulatory substance(s) to the skin of said subject, wherein the immunomodulatory substance(s) administered in step (B₁) is different from the immunomodulatory substance(s) administered in step (B),
wherein step (A), step (B) and/or step (B₁) is performed multiple times, and to said method as such.

Preferably, step (A), step (B) and/or step (B₁) is performed 2 to 3000 times.

More preferably, step (A) is performed 2 to 50 times, even more preferably 2 to 10 times, still even more preferably 2 to 5 times.

More preferably, step (B) is performed 2 to 3000 times, even more preferably 2 to 1000 times, still more preferably 2 to 10 times, still even more preferably 2 to 5 times.

More preferably, step (B₁) is performed 2 to 3000 times, even more preferably 2 to 1000 times, still more preferably 2 to 10 times, still even more preferably 2 to 5 times.

More preferably, step (A), step (B) and step (B₁) are performed 2 to 3000 times, even more preferably 2 to 1000 times, still more preferably 2 to 10 times, still even more preferably 2 to 5 times.

Even more preferably, step (A) is performed 2 to 50 times, still more preferably 2 to 10 times, still even more preferably 2 to 5 times and steps (B) and (B₁) are performed 2 to 3000 times, still more preferably 2 to 1000 times, still even more preferably 2 to 10 times, further preferably 2 to 5 times.

Preferably, in step (A) the skin is of a skin area [a], in step (B) the skin is of a skin area [b] and in step (B₁) the skin is of a skin area [b₁].

More preferably, the multiple performances are carried out in such a way that the area sum of the overlapping skin areas [a]/[b], [a]/[b₁] and/or [a]/[b]/[b₁], even more preferably the area sum of the overlapping skin areas [a]/[b] and/or [a]/[b₁], resulting from the method comprising multiple performances, is enlarged or maximized in comparison to the area sum of the overlapping skin areas [a]/[b], [a]/[b₁] and/or [a]/[b]/[b₁], even more preferably in comparison to the area sum of the overlapping skin areas [a]/[b] and/or [a]/[b₁], of the method, which does not comprise any of the multiple performances. The preference relating to the overlapping skin areas [a]/[b] and/or [a]/[b₁] is particularly for any embodiment of the present invention described herein in which in step (B) IL-4 and in step (B₁) BDNF is administered.

More preferably, at least one of the overlapping skin areas [a]/[b], [a]/[b₁] and/or [a]/[b]/[b₁], even more preferably the overlapping skin areas [a]/[b] and/or [a]/[b₁], resulting from the method comprising multiple performances only partially overlaps and/or does not overlap with at least one other overlapping skin area [a]/[b], [a]/[b₁] and/or [a]/[b]/[b₁], even more preferably one other overlapping skin area [a]/[b] and/or [a]/[b₁], resulting from the method comprising multiple performances. The preference relating to the overlapping skin areas [a]/[b] and/or [a]/[b₁] is particularly for any embodiment of the present invention described herein in which in step (B) IL-4 and in step (B₁) BDNF is administered.

More preferably, particularly in case when in step (B) IL-4 and in step (B₁) BDNF is administered, the method is performed in such a way that none of the skin areas [b] and the skin areas [b₁] do overlap and/or they are distanced apart or the overlap is minimized.

Preferably, the distance of such distanced apart skin areas and/or overlapping skin areas is mentioned in any of the embodiments described herein.

More preferably, particularly in case when in step (B) IL-4 and in step (B₁) BDNF is administered, the method is performed in such a way that no overlapping skin area [b]/[b₁] is formed or the overlapping skin area [b]/[b₁] is minimized. Even more preferably none of the skin areas [b] and/or overlapping skin areas [a]/[b] overlaps with and/or they are distanced apart to any of the skin areas [b₁] and/or overlapping skin areas [a]/[b₁] and/or the area sum of the overlap(s) of these is minimized. Preferably, the distance is defined the same as the distance of the skin area [b] to skin area [b₁] and/or the distance of overlapping skin areas [a]/[b] and/or [a]/[b]/[c] to [a]/[b₁] and/or [a]/[b₁]/[c]. Preferably, the distance of such distanced apart skin areas and/or overlapping skin areas is mentioned in any of the embodiments described herein.

Preferably, step (A), step (B), step (B₁) and any or all of the multiple performances as mentioned in any of the embodiments described herein may be performed in any order, separately, successively, combined together, simultaneously or any combination thereof.

Furthermore, each of step (A), step (B), step (B₁) and any or all of the multiple performances as mentioned in any of the embodiments described herein may be performed independently, successively, simultaneously, separately, combined together or any combination thereof in respect of the one or more of the other steps (A), step (B), step (B₁) and multiple performances.

Preferably, the embodiments relating to whether any of step (A), step (B), step (B₁) and/or step (C) may or may not be completed before the next step is performed apply mutatis mutandis also to step (A), step (B), step (B₁) and the multiple performances as mentioned in any of the embodiments described herein.

If not mentioned otherwise, it is to be understood that any embodiment of the present invention described herein:
for the immunomodulatory substance(s) for use according to the present invention;
for the inflammatory disease, immunological disease and/or autoimmunological disease;
for the immunomodulatory substance(s) in step (B) and/or (B₁); and/or
for steps (A), (B) and/or (B₁), is independently and mutatis mutandis applicable to the still even more preferred aspect of all embodiments of the present invention comprising, that is the above aspect of the present invention which comprises the multiple performances. Thereby it is to be understood that each multiple performance of steps (A), (B) and/or (B₁) may be independently performed according to any of the embodiments as described herein. Hence, for instance, a repetition of step (A) may or may not be performed the same way as another repetition of step (A), for instance in respect of the used agent (e.g. skin-conditioning agent), the concentration, the size of the skin area etc. Same applies to steps (B) and/or (B₁) for instance in respect of the immunomodulatory substance(s), their concentrations, whether administered by topical application or by injection, the size of the skin area etc. Preferably, the repetitions of steps (A), (B) and/or (B₁) are performed the same.

In an still more preferred aspect of all embodiments of present invention described herein, the present invention relates to an immunomodulatory substances and/or a skin-conditioning agent for use in a method for treating and/or preventing an inflammatory disease, immunological disease and/or autoimmunological disease in a subject, wherein the method comprises, preferably consists of, the steps of:
(A) which is selected from one or more of steps (A-0), (A-1), (A-2), (A-3), (A-4), (A-5), (A-6), (A-7) and/or (A-8); and
(B) administering the immunomodulatory substance(s) to the skin of said subject; and
(B₁) administering the immunomodulatory substance(s) to the skin of said subject; and
(C) administering the immunomodulatory substance(s) to the skin of said subject,
wherein the immunomodulatory substance(s) administered in step (B), step (B₁) and step (C) are different from each other,
wherein step (A), step (B), step (B₁) and/or step (C) is performed multiple times, and to said method as such.

Preferably, step (A), step (B), step (B₁) and/or step (C) is performed 2 to 3000 times.

More preferably, step (A) is performed 2 to 50 times, even more preferably 2 to 10 times, still even more preferably 2 to 5 times.

More preferably, step (B) is performed 2 to 3000 times, even more preferably 2 to 1000 times, still more preferably 2 to 10 times, still even more preferably 2 to 5 times.

More preferably, step (B₁) is performed 2 to 3000 times, even more preferably 2 to 1000 times, still more preferably 2 to 10 times, still even more preferably 2 to 5 times.

More preferably, step (C) is performed 2 to 3000 times, even more preferably 2 to 1000 times, still more preferably 2 to 10 times, still even more preferably 2 to 5 times.

More preferably, step (A), step (B), step (B₁) and step (C) are performed 2 to 3000 times, even more preferably 2 to 1000 times, still more preferably 2 to 10 times, still even more preferably 2 to 5 times.

Even more preferably, step (A) is performed 2 to 50 times, still more preferably 2 to 10 times, still even more preferably 2 to 5 times and steps (B), (B₁) and (C) are performed 2 to 3000 times, still more preferably 2 to 1000 times, still even more preferably 2 to 10 times, further preferably 2 to 5 times.

Preferably, in step (A) the skin is of a skin area [a], in step (B) the skin is of a skin area [b] and, in step (B₁) the skin is of a skin area [b₁] and in step (C) the skin is of a skin area [c].

More preferably, the multiple performances are carried out in such a way that the area sum of the overlapping skin areas [a]/[b], [a]/[b₁], [a]/[b]/[c], [a]/[b₁]/[c] and/or [a]/[b]/[b₁]/[c], preferably of the overlapping skin areas [a]/[b], [a]/[b₁], [a]/[b]/[c] and/or [a]/[b₁]/[c], more preferably the overlapping skin areas [a]/[b]/[c] and/or [a]/[b₁]/[c], resulting from the method comprising multiple performances, is enlarged or maximized in comparison to the area sum of the overlapping skin areas [a]/[b], [a]/[b₁], [a]/[b]/[c], [a]/[b₁]/[c] and/or [a]/[b]/[b₁]/[c], preferably of the overlapping skin areas [a]/[b], [a]/[b₁], [a]/[b]/[c] and/or [a]/[b₁]/[c], more preferably the overlapping skin areas [a]/[b]/[c] and/or [a]/[b₁]/[c], of the method, which does not comprise any of the multiple performances. The preference relating to the overlapping skin areas [a]/[b], [a]/[b₁], [a]/[b]/[c] and/or [a]/[b₁]/[c] and more preferably the overlapping skin areas [a]/[b]/[c] and/or [a]/[b₁]/[c] is particularly for any embodiment of the present invention described herein in which in step (B) IL-4 and in step (B₁) BDNF is administered.

More preferably, at least one of the overlapping skin areas [a]/[b], [a]/[b₁], [a]/[b]/[c], [a]/[b₁]/[c] and/or [a]/[b]/[b₁]/[c], preferably of the overlapping skin areas [a]/[b], [a]/[b₁], [a]/[b]/[c] and/or [a]/[b₁]/[c], preferably of the overlapping skin areas [a]/[b]/[c] and/or [a]/[b₁]/[c], resulting from the method comprising multiple performances only partially overlaps and/or does not overlap with at least one other overlapping skin area [a]/[b], [a]/[b₁], [a]/[b]/[c], [a]/[b₁]/[c] and/or [a]/[b]/[b₁]/[c], preferably one other overlapping skin area [a]/[b], [a]/[b₁], [a]/[b]/[c] and/or [a]/[b₁]/[c], more preferably one other overlapping skin areas [a]/[b]/[c] and/or [a]/[b₁]/[c], resulting from the method comprising multiple performances. The preference relating to the overlapping skin areas [a]/[b], [a]/[b₁], [a]/[b]/[c] and/or [a]/[b₁]/[c] and more preferably the overlapping skin areas [a]/[b]/[c] and/or [a]/[b₁]/[c] is particularly for any embodiment of the present invention described herein in which in step (B) IL-4 and in step (B₁) BDNF is administered.

More preferably, is particularly for any embodiment of the present invention described herein in which in step (B) IL-4 and in step (B₁) BDNF is administered, the method is performed in such a way that none of the skin areas [b] and the skin areas [b₁] do overlap and/or they are distanced apart or the overlap is minimized. Preferably, the distance of such distanced apart skin areas and/or overlapping skin areas mentioned in any of the embodiments described herein.

More preferably, is particularly for any embodiment of the present invention described herein in which in step (B) IL-4 and in step (B₁) BDNF is administered, the method is performed in such a way that no overlapping skin area [b]/[b₁] is formed or the overlapping skin area [b]/[b₁] is minimized. Even more preferably none of the skin areas [b], overlapping skin areas [a]/[b] or overlapping skin areas [a]/[b]/[c] overlap with and/or they are distanced apart to any of the skin areas [b₁], overlapping skin areas [a]/[b₁] and/or overlapping skin areas [a]/[b₁]/[c] and/or the area sum of the overlap(s) of these is minimized. Preferably, the distance of such distanced apart skin areas and/or overlapping skin areas is mentioned in any of the embodiments described herein.

Preferably, step (A), step (B), step (B₁) and any or all of the multiple performances as mentioned in any of the embodiments described herein may be performed in any order, separately, successively, combined together, simultaneously or any combination thereof.

Furthermore, each of step (A), step (B), step (B₁) and any or all of the multiple performances as mentioned in any of the embodiments described herein may be performed independently, successively, simultaneously, separately, combined together or any combination thereof in respect of the one or more of the other steps (A), steps (B), steps (B₁) and multiple performances.

Preferably, the embodiments relating to whether any of step (A), step (B), step (B₁) and/or step (C) may or may not be completed before the next step is performed apply mutatis mutandis also to step (A), step (B), step (B₁), step (C) and the multiple performances as mentioned in any of the embodiments described herein.

If not mentioned otherwise, it is to be understood that any embodiment of the present invention described herein:
for the immunomodulatory substance(s) for use according to the present invention;
for the inflammatory disease, immunological disease and/or autoimmunological disease;
for the immunomodulatory substance(s) in step (B), (BD and/or (C); and/or
for steps (A), (B), (B₁) and/or (C),
is independently and mutatis mutandis applicable to the still even more preferred aspect of all embodiments of the present invention comprising, that is the above aspect of the present invention which comprises the multiple performances. Thereby it is to be understood that each multiple performance of steps (A), (B), (B₁) and/or (C) may be independently performed according to any of the embodiments as described herein. Hence, for instance, a repetition of step (A) may or may not be performed the same way as another repetition of step (A), for instance in respect of the used agent (e.g. skin-conditioning agent), the concentration, the size of the skin area etc. Same applies to steps (B), (B₁) and/or (C), for instance in respect of the immunomodulatory substance(s), their concentrations, whether administered by topical application or by injection, the size of the skin area etc. Preferably, the repetitions of steps (A), (B), (B₁) and/or (C) are performed the same.

In an still even more preferred aspect of all embodiments of present invention described herein, the present invention relates to an immunomodulatory substances and/or a skin-conditioning agent for use in a method for treating and/or preventing an inflammatory disease, immunological disease and/or autoimmunological disease in a subject,
and the method comprises, preferably consists of:
   a first set of steps, comprising, preferably consisting of:
   (A) which is selected from one or more of steps (A-0), (A-1), (A-2), (A-3), (A-4), (A-5), (A-6), (A-7) and/or (A-8); and
   (B) administering the immunomodulatory substance(s) to the skin of said subject; and
   (C) administering the immunomodulatory substance(s) to the skin of the subject, and
   a second set of steps comprising, preferably consisting of:
   (A) which is selected from one or more of steps (A-0), (A-1), (A-2), (A-3), (A-4), (A-5), (A-6), (A-7) and/or (A-8); and
   (B₁) administering the immunomodulatory substance(s) to the skin of said subject; and
   (C) administering the immunomodulatory substance(s) to the skin of the subject,
wherein the immunomodulatory substance(s) administered in step (B₁) is different from the immunomodulatory substance(s) administered in step (B),
wherein in the first set of steps or n the second set of steps or in both step (A), step (B), step (B₁) and/or step (C) is performed multiple times,
and to said method as such.

Preferably, in the first set of steps or in the second set of steps or in both step (A), step (B), step (B₁) and/or step (C) is performed, preferably independently performed, 2 to 3000 times.

More preferably, in the first set of steps or in the second set of steps or n both step (A) is performed 2 to 50 times, even more preferably 2 to 10 times, still more preferably 2 to 5 times.

More preferably, in the first set of steps or in the second set of steps or in both step (B) is performed 2 to 3000 times, even more preferably 2 to 1000 times, still more preferably 2 to 10 times, still even more preferably 2 to 5 times.

More preferably, in the first set of steps or in the second set of steps or in both step (B₁) is performed 2 to 3000 times, even more preferably 2 to 1000 times, still more preferably 2 to 10 times, still even more preferably 2 to 5 times.

More preferably, in the first set of steps or in the second set of steps or in both step (C) is performed 2 to 3000 times, even more preferably 2 to 1000 times, still more preferably 2 to 10 times, still even more preferably 2 to 5 times.

More preferably, in the first set of steps or in the second set of steps or in both step (A), step (B), step (B₁) and step (C) are performed 2 to 3000 times, even more preferably 2 to 1000 times, still more preferably 2 to 10 times, still even more preferably 2 to 5 times.

Even more preferably, in the first set of steps or in the second set of steps or in both step (A) is performed 2 to 50 times, still more preferably 2 to 10 times, still even more preferably 2 to 5 times and steps (B) and (C) are performed 2 to 3000 times, still more preferably 2 to 1000 times, still even more preferably 2 to 10 times, further preferably 2 to 5 times.

Even more preferably, in the first set of steps or in the second set of steps or in both step (A) is performed 2 to 50 times, still more preferably 2 to 10 times, still even more preferably 2 to 5 times and steps (B₁) and (C) are performed 2 to 3000 times, still more preferably 2 to 1000 times, still more preferably 2 to 10 times, further preferably 2 to 5 times.

Preferably, the immunomodulatory substance(s) administered in each of steps (C) are the same or different, more preferably are the same.

If not mentioned otherwise, it is to be understood that any embodiment of step (A) described herein is independently applicable to each of steps (A). Hence, steps (A) may or may not the same, for instance in respect to the way of generation, the concentration, the size of the skin area etc. More preferably, steps (A) are the same. If not mentioned otherwise, it is to be understood that any embodiment of step (C) described herein is independently applicable to each of steps (C). Hence, steps (C) may or may not be the same, for instance in respect to the immunomodulatory substance(s), their concentrations, whether administered by topical application or by injection, the size of the skin area etc. More preferably, steps (C) are the same.

Preferably,
in the first set of steps, in step (A) the skin is of a skin area [a], in step (B) the skin is of a skin area [b] and in step (C) the skin is of a skin area [c], and
in the second set of steps, in step (A) the skin is of a skin area [a], in step (B) the skin is of a skin area [b] and in step (B₁) the skin is of a skin area [b₁].

More preferably, the multiple performances are carried out in such a way that the area sum of the overlapping skin areas [a]/[b], [a]/[b₁], [a]/[b]/[c], [a]/[b₁]/[c] and/or [a]/[b]/[b₁]/[c], preferably of the overlapping skin areas [a]/[b], [a]/[b₁], [a]/[b]/[c] and/or [a]/[b₁]/[c], more preferably the overlapping skin areas [a]/[b]/[c] and/or [a]/[b₁]/[c], resulting from the method comprising multiple performances, are enlarged or maximized in comparison to the area sum of the overlapping skin areas [a]/[b], [a]/[b₁], [a]/[b]/[c], [a]/[b₁]/[c] and/or [a]/[b]/[b₁]/[c], preferably of the overlapping skin areas [a]/[b], [a]/[b₁], [a]/[b]/[c] and/or [a]/[b₁]/[c], more preferably the overlapping skin areas [a]/[b]/[c] and/or [a]/[b₁]/[c], of the method, which does not comprise any of the multiple performances. The preference relating to the overlapping skin areas [a]/[b], [a]/[b₁], [a]/[b]/[c] and/or [a]/[b₁]/[c] and more preferably the overlapping skin areas [a]/[b]/[c] and/or [a]/[b₁]/[c] is particularly for any embodiment of the present invention described herein in which in step (B) IL-4 and in step (B₁) BDNF is administered.

More preferably, at least one of the overlapping skin areas [a]/[b], [a]/[b₁], [a]/[b]/[c], [a]/[b₁]/[c] and/or [a]/[b]/[b₁]/[c], preferably of the overlapping skin areas [a]/[b], [a]/[b₁], [a]/[b]/[c] and/or [a]/[b₁]/[c], preferably of the overlapping skin areas [a]/[b]/[c] and/or [a]/[b₁]/[c], resulting from the method comprising multiple performances only partially overlaps and/or does not overlap with at least one other overlapping skin area [a]/[b], [a]/[b₁], [a]/[b]/[c], [a]/[b₁]/[c] and/or [a]/[b]/[b₁]/[c], preferably one other overlapping skin area [a]/[b], [a]/[b₁], [a]/[b]/[c] and/or [a]/[b₁]/[c], more preferably one other overlapping skin areas [a]/[b]/[c] and/or [a]/[b₁]/[c], resulting from the method comprising multiple performances. The preference relating to the overlapping skin areas [a]/[b], [a]/[b₁], [a]/[b]/[c] and/or [a]/[b₁]/[c] and more preferably the overlapping skin areas [a]/[b]/[c] and/or [a]/[b₁]/[c] is particularly for any embodiment of the present invention described herein in which in step (B) IL-4 and in step (B₁) BDNF is administered.

More preferably, particularly for any embodiment of the present invention described herein in which in step (B) IL-4 and in step (B₁) BDNF is administered, the method is performed in such a way that none of the skin areas [b] and the skin areas [b₁] do overlap and/or they are distanced apart or the overlap is minimized. Preferably, the distance of such distanced apart skin areas and/or overlapping skin areas is mentioned in any of the embodiments described herein.

More preferably, is particularly for any embodiment of the present invention described herein in which in step (B) IL-4 and in step (B) BDNF is administered, the method is performed in such a way that no overlapping skin area [b]/[b₁] is formed or the overlapping skin area [b]/[b₁] is minimized. Even more preferably none of the skin areas [b], overlapping skin areas [a]/[b] or overlapping skin areas [a]/[b]/[c] overlap with and/or they are distanced apart to any of the skin areas [b₁], overlapping skin areas [a]/[b₁] and/or overlapping skin areas [a]/[b₁]/[c] and/or the area sum of the overlap(s) of these is minimized. Even more preferably none of the overlapping skin areas [a]/[b]/[c] overlap with and/or they are distanced apart to any of the overlapping skin areas [a]/[b₁]/[c] and/or the area sum of the overlap(s) of these is minimized. Preferably, the distance of such distanced apart skin areas and/or overlapping skin areas is mentioned in any of the embodiments described herein.

If not mentioned otherwise, it is to be understood that any of the embodiments as described herein:
for the immunomodulatory substance(s) for use according to the present invention;
for the inflammatory disease, immunological disease and/or autoimmunological disease;
for the immunomodulatory substance(s) in steps (B), (B₁) and/or (C); and/or
for steps (A), (B), (B₁) and/or (C),
is independently and mutatis mutandis applicable to the still even more preferred aspect of all embodiments of the present invention comprising, that is the above aspect of the present invention which comprises the multiple performances. Thereby it is to be understood that each multiple performance of steps (A), (B), (B₁) and/or (C) may be independently performed according to any of the embodiments as described herein. Hence, for instance, a repetition of step (A) may or may not be performed the same way as another repetition of step (A), for instance in respect of the used agent (e.g. skin-conditioning agent), the concentration, the size of the skin area etc. Same applies to steps (B), (B₁) and/or (C) for instance in respect of the immunomodulatory substance(s), their concentrations, whether administered by topical application or by injection, the size of the skin area etc. Preferably, the repetitions of steps (A), (B), (B₁) and/or (C) are performed the same.

Preferably, the embodiments relating to whether any of step (A), step (B), step (B₁) and/or step (C) may or may not be completed before the next step is performed apply mutatis mutandis also to step (A), step (B), step (B₁), step (C), multiple performances as mentioned in any of the embodiments described herein.

Preferably, in any of the embodiments described herein:
the overlapping skin area [a]/[b] and/or the area sum of the overlapping skin areas [a]/[b];
the overlapping skin area [b]/[c] and/or the area sum of the overlapping skin areas [b]/[c];
the overlapping skin area [a]/[c] and/or the area sum of the overlapping skin areas [a]/[c];
the overlapping skin area [a]/[b]/[c] and/or the area sum of the overlapping skin areas [a]/[b]/[c];
the overlapping skin area [a]/[b₁] and/or the area sum of the overlapping skin areas [a]/[b₁];
the overlapping skin area [b₁]/[c] and/or the area sum of the overlapping skin areas [b₁]/[c];
the overlapping skin area [a]/[b₁]/[c] and/or the area sum of the overlapping skin areas [a]/[b₁]/[c];
the overlapping skin area [a]/[b]/[b₁]/[c] and/or the area sum of the overlapping skin areas [a]/[b]/[b₁]/[c];
the area sum of the overlapping skin areas [a]/[b] and/or [a]/[b]/[c];
the area sum of the overlapping skin areas [a]/[b] and/or [a]/[b₁];
the area sum of the overlapping skin areas [a]/[b], [a]/[b₁], [a]/[b]/[c] and/or [a]/[b₁]/[c];
the area sum of the overlapping skin areas [a]/[b]/[c] and/or [a]/[b₁]/[c] the overlapping skin area [a]/[b] and, if applicable, overlapping skin areas [a]/[b]/[c], [a]/[b₁] and/or [a]/[b₁]/[c]; and/or
the area sum of the overlapping skin area [a]/[b] and, if applicable overlapping skin areas [a]/[b]/[c], [a]/[b₁] and/or [a]/[b₁]/[c],
independently may have any size sufficient to achieve the beneficial effects. More preferably, such skin area and/or such area sum is/are enlarged or maximized. The skin area and/or the area sum and may have a size of even about 1 m² or less. Preferably, the skin area is 1,000 cm² or less, more preferably 500 cm² or less, even more preferably 100 cm² or less, still more preferably 50 cm² or less, still even more preferably 25 cm² or less, further preferably 10 cm², even further preferably 5 cm² or less. Usually, the skin area is not smaller than 0.5 cm². Hence, a lower limit for the skin area is preferably 0.5 cm² or more, more preferably 1 cm² or more, still more preferably 2 cm² or more. The skin area and/or the area sum as mentioned in any of the embodiments is preferably measured as described in the Materials and Methods' section ‘Measurement of areas and distances relating to the skin’.

Preferably, the method, if applicable, including any or all of the multiple performances are comprised, as mentioned in any of the embodiments described herein is performed at a frequency of once per day or less often, more preferably once per 2 days or less often, even more preferably once per 3 days or less often, still more preferably once per 4 days or less often, still even more preferably once per 5 days or less often, further preferably once per week or less often, further preferably once per 2 weeks or less often, even further preferably once per month or less often. Usually, there is no upper limit for the frequency as long the beneficial effects can be achieved or is maintained. Furthermore, it may be in the interest of the treated subject and in respect of effort and cost efficiency to perform the method as seldom as possible. Nevertheless, an upper limit may be, preferably, is that the method is performed at a frequency of once per two months or more often, more preferably, once per 6 weeks or more often. Preferably, the method is performed at a frequency of once per day to one per two months, more preferably once per 2 days to once per two months, even more preferably once per 2 days to once per months, still more preferably once per 3 days to once per 6 weeks, still even more preferably once per 4 days to once per 6 weeks, furthermore preferably once per 5 days to once per month, even furthermore once per week to once per 2 weeks.

Preferably, steps (A) and (B); steps (A) and (C); steps (A), (B) and (C); steps (A), (B) and (B₁); and/or steps (A), (B), (B₁) and (C), if applicable, including any or all of the multiple performances are comprised, are performed at a frequency of once per day or less often, more preferably once per 2 days or less often, even more preferably once per 3 days or less often, still more preferably once per 4 days or less often, still even more preferably once per 5 days or less often, further preferably once per week or less often, still further preferably once per 2 weeks or less often, more preferably once per month or less often. Usually, there is no upper limit for the frequency as long the beneficial effects can be achieved, however, an upper limit may be, preferably, is that steps (A) and (B), more preferably, steps (A), (B) and (C); steps (A), (B) and (B₁); and/or steps (A), (B), (B₁) and (C), if applicable, including any or all of the multiple performances are comprised, are performed at a frequency of once per two months or more often, more preferably, once per 6 weeks or more often. Preferably, the steps (A) and (B), more preferably steps (A), (B) and (C); steps (A), (B) and (B₁); and/or steps (A), (B), (B₁) and (C), if applicable, including any or all of the multiple performances are comprised, are performed at a frequency of once per day to one per two months, more preferably once per 2 days to once per two months, even more preferably once per 2 days to once per months, still more preferably once per 3 days to once per 6 weeks, still even more preferably once per 4 days to once per 6 weeks, furthermore preferably once per 5 days to once per month, even furthermore once per week to once per 2 weeks.

Preferably, the method, if applicable, including any or all of the multiple performances, as mentioned in any of the embodiments described herein is performed within 5 hours or less, more preferably within 3 hours or less, even more preferably within 1 hour or less, still more preferably within 0.5 hours or less, even further preferred within 15 min or less, still further preferably within 5 min or less, still even further preferably, within 1 min or less. There is no lower limit for the period of time within which the method may be performed and it may be in the interest of the treated subject to keep the period of time as short as possible. For instance in case of using a composition like a crème comprising all active ingredients of for instance step (A), (B) and (C), the application of the crème topically on the skin may only take a second, or in case of using an injection pen it may take even less. When using an injection pen, it may be even shorter. Preferably the method is performed within 5 hours or less and 0.05 sec or more, more preferably within 3 hours or less and 0.5 sec or more, even more preferably within 1 hour or less and 1 sec or more, still more preferably within 0.5 hours or less 1 sec or more, even further preferred within 15 min or less 1 sec and/or more, still further preferably within 5 min or less and 2 sec or more.

As already stated above, in any of the embodiments described herein it is to be understood that it is preferred that the individual effects generated by steps (A), (B), (B₁) and/or (C) should at least partially and/or totally overlap in time as well as spatially, for instance e.g. to be mutually dependent, supportive, interactive, complementary, additive, synergetic and/or to act by any other way together. Hence, it is to be understood that the method should be performed accordingly.

Hence, preferably, the method, if applicable, including any or all of the multiple performances are comprised, as mentioned in any of the embodiments described herein are performed such a way that the accumulation, vasodilation, increased blood volume, increased sO₂ and/or increased rHb, increased temperature and/or redness generated by step (A) partially and/or totally overlaps in time with the presence of the immunomodulatory substance(s), preferably the increased concentration and/or the amount of the immunomodulatory substance(s), within the skin generated by administering the immunomodulatory substance(s) in step (B), (B₁) and/or (C) and, even more preferably, if applicable in some or all of the multiple performances. Preferably, the immunomodulatory substance(s), preferably the increased concentration and/or amount of the immunomodulatory substance(s), present within the skin is an effective concentration and/or amount of the immunomodulatory substance(s), wherein such amount may vary between individual subjects. Similarly, the overlap in time may vary from subject to subject.

Preferably, steps (A) and (B); steps (A) and (C); steps (A), (B) and (C); steps (A), (B) and (B₁); and/or steps (A), (B), (B₁) and (C), if applicable, including any or all of the multiple performances are comprised, in any of the embodiments described herein are performed within 5 hours or less, more preferably within 3 hours or less, even more preferably within 1 hour or less, still more preferably within 0.5 hours or less, still even more preferred within 15 min or less, further preferably within 5 min or less, even further preferably, within 1 min or less. There is no lower limit for the period of time within which the method may be performed and it may be in the interest of the treated subject to keep the period of time as short as possible. For instance in case of using a composition like a crème comprising all active ingredients of for instance step (A), (B) and (C), the application of the crème topically on the skin may only take a second, or in case of using an injection pen it may take even less. Preferably steps (A) and (B), more preferably steps (A), (B) and (C); steps (A), (B) and (B₁); and/or steps (A), (B), (B₁) and (C), if applicable, including any or all of the multiple performances are comprised, are performed within 5 hours or less and 0.05 sec or more, more preferably within 3 hours or less and 0.5 sec or more, even more preferably within 1 hour or less and 1 sec or more, still more preferably within 0.5 hours or less 1 sec or more, even further preferred within 15 min or less 1 sec and/or more, still further preferably within 5 min or less and 2 sec or more.

Preferably, steps (A) and (B); steps (A) and (C); steps (A), (B) and (C); steps (A), (B) and (B₁); and/or steps (A), (B), (B₁) and (C), if applicable, including any or all of the multiple performances are comprised, as mentioned in any of the embodiments described herein are performed such a way that the accumulation, vasodilation, increased blood volume, increased sO₂ and/or increased rHb, increased temperature and/or redness generated by step (A) (e.g. particularly by below steps (A-6) to (A-8)), depending on what is applicable, partially overlaps and/or totally overlaps in time with the presence of the immunomodulatory substance(s) within the skin administered in one or more, preferably in all of steps (B), (B₁) and/or (C) and, even more preferably, if applicable in some or all of the multiple performances. More preferably, the amount of the immunomodulatory substance(s) present within the skin is an effective amount of the immunomodulatory substance(s), wherein such amount may vary between individual subjects.

Preferably, it is to be understood that the individual effects generated by steps (A), (B), (B₁) and/or (C) partially and/or totally overlap in time, for instance e.g. to be mutually dependent, supportive, interactive, complementary, additive, act together and/or synergetic.

Preferably, steps (A) and (B); steps (A) and (C); steps (A), (B) and (C); steps (A), (B) and (B₁); and/or steps (A), (B), (B₁) and (C), if applicable, including any or all of the multiple performances are comprised, as mentioned in any of the embodiments described herein are performed such a way that the accumulation, vasodilation, increased blood volume, increased sO₂ and/or increased rHb, increased temperature and/or redness generated by step (A) partially and/or totally overlaps in time with the presence of the immunomodulatory substance(s), preferably the increased concentration and/or amount of the immunomodulatory substance(s) within the skin generated by administering the immunomodulatory substance(s) in step (B), (B₁) and/or (C) and, even more preferably, if applicable in some or all of the multiple performances. Preferably, the immunomodulatory substance(s), preferably the increased concentration and/or amount of the immunomodulatory substance(s), present within the skin is an effective concentration and/or amount of the immunomodulatory substance(s), wherein such amount may vary between individual subjects. Similarly, the overlap in time may vary from subject to subject.

Preferably, the overlap in time, whether it is a partial or total overlap, is sufficient to achieve the beneficial effects and may vary between individual subjects. More preferably it is 2 min or more, even more preferably 5 min or more, still more preferably 10 min or more, still even more preferably 15 min or more, further preferably 20 min or more, even further preferably 0.5 hours or more, still further preferably 0.75 hours or more, still even further preferably 1 h or more. There is no upper limit for the overlap in time, however, usually it is 48 hours or less. Preferably, the overlap in time, whether it is a partial or total overlap, is in the range of 2 min or more and 48 hours or less, more preferably 5 min or more and 24 hours or less, even more preferably 10 min or more and 1 day or less, still more preferably 15 min or more and 12 hours or less, still even more preferably 20 min or more and 12 hours or less, further preferably 0.5 hours or more and 6 hours or less, even further preferably 0.75 hours or more and 3 hours or less, still more preferably 1 h or more and 3 hours or less.

Preferably, as stated above, step (A) is selected from one or more of steps:
(A-1) generating a vasodilation of the capillaries within the skin of the subject, wherein it is to be understood that preferably any embodiment or definition thereof which is described under the outline note ‘As further regards step (A-1) for any of the embodiments as described herein’ is independently and mutatis mutandis applicable;
(A-2) generating an increased blood volume within the skin of the subject, wherein it is to be understood that preferably any embodiment or definition thereof which is described under the outline note ‘As further regards step (A-2) for any of the embodiments as described herein’ is independently and mutatis mutandis applicable;
(A-3) generating an increased sO₂ and/or an increased rHb within the skin, wherein it is to be understood that preferably any embodiment or definition thereof which is described under the outline note ‘As further regards step (A-3) for any of the embodiments as described herein’ is independently and mutatis mutandis applicable;
(A-4) generating an increased temperature on the skin of the subject, wherein it is to be understood that preferably any embodiment or definition thereof which is described under the outline note ‘As further regards step (A-4) for any of the embodiments as described herein’ is independently and mutatis mutandis applicable;
(A-5) generating a redness on the skin of the subject, wherein it is to be understood that preferably any embodiment or definition thereof which is described under the outline note ‘As further regards step (A-5) for any of the embodiments as described herein’ is independently and mutatis mutandis applicable;
(A-6) administering conditioning energy to the skin of the subject, wherein it is to be understood that preferably any embodiment or definition thereof which is described under the outline note ‘As further regards step (A-6) for any of the embodiments as described herein’ is independently and mutatis mutandis applicable;
(A-7) administering a skin-conditioning agent to the skin of the subject, wherein it is to be understood that preferably any embodiment or definition thereof which is described under the outline note ‘As further regards step (A-7) for any of the embodiments as described herein’ is independently and mutatis mutandis applicable; and/or
(A-8) administering PBMCs to the skin the skin of the subject, preferably into the skin of the skin area, wherein it is to be understood that preferably any embodiment or definition thereof which is described under the outline note ‘As further regards step (A-8) for any of the embodiments as described herein’ is independently and mutatis mutandis applicable.

Preferably, steps (A-0) to (A-8) may not be mutually exclusive.

More preferably, the further step is selected from one or more of (A-0), (A-1), (A-2), (A-3), (A-4), (A-5), (A-6) and/or (A-8), even more preferably (A-1), (A-2), (A-3) and/or (A-4).

If not mentioned otherwise, it is to be understood that any embodiment of the present invention described herein relating to step (A) is independently and mutatis mutandis applicable to step (A-0), (A-1), (A-2), (A-3), (A-4), (A-5), (A-6), (A-7) and/or (A-8) in any of the embodiments described herein.

Particularly, any of the embodiments as described herein for step (A) and relating to:
the inflammatory disease, immunological disease and/or autoimmunological disease;
multiple performances;
the skin area [a];
the application area [a];
overlapping skin areas;
overlapping application areas;
immunological inactivity and/or unchallenge, the special distance to any site of immunological activity and/or challenge;
multiple performances;
the fact that the steps of the method may be performed in any order, separately, successively, combined together, simultaneously or any combination thereof;
whether any of step (A), step (B), step (B₁) and/or step (C) may or may not be completed before the next step is performed;
steps (B), (B₁) and/or (C),
independently applies mutatis mutandis to step (A-0), (A-1), (A-2), (A-3), (A-4), (A-5), (A-6), (A-7) and/or (A-8) in any of the embodiments described herein.

Preferably, in any of the embodiments described herein, a skin area and/or application area in step (A-0), (A-1), (A-2), (A-3), (A-4), (A-5), (A-6), (A-7) and/or (A-8) is a skin area [a] and an application area [a], respectively.

Preferably, any of the embodiments as described herein relating to step (A) and a skin area or skin area [a] is independently and mutatis mutandis applicable to the skin area [a] of any of step (A-0), (A-1), (A-2), (A-3), (A-4), (A-5), (A-6), (A-7) and/or (A-8).

Preferably, any of the embodiments as described herein relating to step (A) and an application area or an application area [a] is independently and mutatis mutandis applicable to the application area [a] of any of step (A-0), (A-1), (A-2), (A-3), (A-4), (A-5), (A-6), (A-7) and/or (A-8).

Preferably, in any of the embodiments described herein, a skin area [a], application area [a] and/or respective overlapping skin and application areas of any of steps (A), (A-0), (A-1), (A-2), (A-3), (A-4), (A-5), (A-6), (A-7) and (A-8) may be the same or a different to a skin area [a], application area [a] and/or respective overlapping skin and application areas of any other of steps (A), (A-0), (A-1), (A-2), (A-3), (A-4), (A-5), (A-6), (A-7) and (A-8). Hence, they may independently be distanced apart, partially overlapping, totally overlapping and/or congruent.

In the following, steps (A-0) to (A-8) are described in further detail.

If not mentioned otherwise, it is to be understood that any of the embodiments mentioned under the following outline notes:
‘As further regards step (A-0) for any of the embodiments as described herein’,
‘As further regards step (A-1) for any of the embodiments as described herein’,
‘As further regards step (A-2) for any of the embodiments as described herein’,
‘As further regards step (A-3) for any of the embodiments as described herein’,
‘As further regards step (A-4) for any of the embodiments as described herein’,
‘As further regards step (A-5) for any of the embodiments as described herein’,
‘As further regards step (A-6) for any of the embodiments as described herein’,
‘As further regards step (A-7) for any of the embodiments as described herein’, and/or
‘As further regards step (A-8) for any of the embodiments as described herein’,
are independently applicable to each other and/or combinable with each other and with any of the embodiments as described herein.

The outline notes merely serve to structure the text and are not intended to have any delimiting or restrictive meaning beyond that. This applies to all outline notes throughout the entire description.

As further regards step (A-0) for any of the embodiments as described herein

As stated above, step (A-0) requires generating an accumulation of PBMCs within the skin of the subject.

Preferably, in any of the embodiments described herein and particularly in step (A-0), the accumulation of PBMCs is generated within a sub-topical layer of the skin, more preferably within the dermis and/or the subcutis, preferably of the skin of the skin area.

Preferably, in any of the embodiments described herein and particularly in step (A-0), the accumulation of PBMCs is generated within a sub-topical layer of the skin, more preferably within the dermis and/or the subcutis, preferably of the skin of the skin area.

Preferably, in any of the embodiments described herein and particularly in step (A-0), the accumulation of PBMCs is generated within the lumen of the capillaries of the skin and/or within the skin tissue. More preferably, in case of any of steps (A-1) to (A-7) below, the accumulation of PBMCs is generated within the lumen of the capillaries of the skin and/or within the skin tissue, more preferably within the lumen of the capillaries. More preferably, in case of step (A-8) below, the PBMCs may be injected and the accumulation of PBMCs is then generated directly within the skin tissue.

More preferably, the accumulation of PBMCs is generated within a sub-topical layer of the skin, even more preferably within the dermis and/or subcutis, still more preferably the dermis, of the skin, preferably of the skin area. Still more preferably, the accumulation of PBMCs is generated within the lumen of the capillaries and/or the skin tissue of a sub-topical layer, still even more preferably within the lumen of the capillaries and/or the skin tissue of the dermis and/or subcutis, of the skin, preferably of the skin of the skin area.

Preferably, in any of the embodiments described herein and particularly in step (A-0), the PBMCs are blood-derived PBMCs.

Preferably, in any of the embodiments described herein and particularly in step (A-0), the accumulation of PBMCs is an accumulation of lymphocytes, more preferably B-cells and/or T-cells, even more preferably T-cells, still more preferably naïve PBMCs, still even more preferably naïve lymphocytes, further preferably naïve B-cells and/or naïve T-cell, even further preferably naïve T-cells.

Preferably, in any of the embodiments described herein and particularly in step (A-0), the generating of the accumulation of PBMCs may be, preferably is accomplished by any means, method(s), substance(s) and/or procedure(s) as long as it is suitable and/or configured to generate an accumulation of PBMCs within the skin and/or without damaging the skin like burning, causing lesions, rupture of capillaries and/or reflexive closure of the capillaries.

As stated already elsewhere, and without wishing to be bound to theory, it is believed that PBMCs, in particular lymphocytes, more preferably naïve PBMCs, even more preferably naïve lymphocytes, still more preferably naïve B-cells and/or naïve T-cells, and/or still even more preferably naïve T-cells are not present or are at least not present in effective amounts within the skin capillaries and hence within the skin. Hence, already the presence of PBMCs, preferably lymphocytes, more preferably naïve PBMCs, even more preferably naïve lymphocytes, still more preferably naïve B-cells and/or naïve T-cells, still even more preferably naïve T-cells within the skin may, preferably, may be regarded as accumulation of PBMCs, preferably lymphocytes, more preferably naïve PBMCs, even more preferably naïve lymphocytes, still more preferably naïve B-cells and/or naïve T-cells, still even more preferably naïve T-cells in step (A-0). Preferably, the accumulation of the PBMCs may be indicated by an increase in the amount of PBMCs within the skin, preferably a sub-topical layer of the skin, more preferably within the dermis and/or the subcutis of the skin, more preferably within the lumen of the capillaries of the skin, even more preferably within the lumen of the capillaries of the dermis and/or the subcutis of the skin.

Already the presence of the PBMCs within the skin, preferably a sub-topical layer of the skin, more preferably within the dermis and/or the subcutis of the skin, may be regarded as an increase of the amount and/or an accumulation of the PBMCs. Preferably, the accumulation of the PBMCs may be indicated by an increase in the amount of PBMCs, preferably lymphocytes, more preferably naïve PBMCs, even more preferably naïve lymphocytes, still more preferably naïve B-cells and/or naïve T-cells, still even more preferably naïve T-cells, within the skin, preferably a sub-topical layer of the skin, more preferably within the dermis and/or the subcutis of the skin.

The presence and/or quantification of the amount of PBMCs, preferably of lymphocytes, more preferably naïve PBMCs, even more preferably naïve lymphocytes, still more preferably naïve B-cells and/or naïve T-cells, still even more preferably naïve T-cells, preferably within the skin, preferably a sub-topical layer of the skin, more preferably within the dermis and/or the subcutis of the skin, and/or preferably within the lumen of the capillaries of the skin, more preferably within the lumen of the capillaries of the dermis and/or the subcutis of the skin, can be measured by any suitable method known to the person skilled in the art suitable to count and/or otherwise quantify such cells within the skin.

Preferably, in any of the embodiments described herein and particularly in step (A-0), the accumulation of PBMCs within the skin is to an extent and/or a duration suitable to achieve the beneficial effects, is pharmaceutically acceptable and/or does not cause lesions and/or a damaging of skin like burning, causing lesions, rupture of capillaries and/or reflexive closure of the capillaries. Preferably, in any of the embodiments described herein and particularly in step (A-0), the accumulation of PBMCs within the skin may to any extent and/or any duration as long as it is pharmaceutically acceptable and does not cause lesions and/or a damaging of skin like burning, causing lesions, rupture of capillaries and/or reflexive closure of the capillaries.

Preferably, in any of the embodiments described herein and particularly in step (A-0),
the accumulation of PBMCs is indicated by; and/or
the accumulation of PBMCs is to generate, preferably the accumulation of PBMCs is generated to an extent and/or a duration is suitable and/or sufficient to generate:
   a vasodilation of the capillaries within the skin of the subject, wherein it is to be understood that preferably any embodiment or definition thereof which is described under the outline note ‘As further regards step (A-1) for any of the embodiments as described herein’ is independently and mutatis mutandis applicable; and/or
   an increased blood volume within the skin, wherein it is to be understood that preferably any embodiment or definition thereof which is described under the outline note ‘As further regards step (A-2) for any of the embodiments as described herein’ is independently and mutatis mutandis applicable; and/or
   an increased sO₂ and/or an increased rHb within the skin, wherein it is to be understood that preferably any embodiment or definition thereof which is described under the outline note ‘As further regards step (A-3) for any of the embodiments as described herein’ is independently and mutatis mutandis applicable; and/or
   an increased temperature on the skin, wherein it is to be understood that preferably any embodiment or definition thereof which is described under the outline note ‘As further regards step (A-4) for any of the embodiments as described herein’ is independently and mutatis mutandis applicable; and/or
   a redness on the skin, wherein it is to be understood that preferably any embodiment or definition thereof which is described under the outline note ‘As further regards step (A-5) for any of the embodiments as described herein’ is independently and mutatis mutandis applicable.

Preferably, the accumulation and/or quantification of the amount of PBMCs, lymphocytes, T-cells, and/or naïve T-cells, more preferably naïve PBMCs, even more preferably naïve lymphocytes, still more preferably naïve B-cells and/or naïve T-cells, still even more preferably naïve T-cells, in any of the embodiments described herein can be measured by any method known to the person skilled in the art suitable to count and/or otherwise quantify such cells within the skin.

More preferably, in any of the embodiments described herein and particularly in step (A-0), the accumulation of PBMCs within the skin is measured by any suitable method known to the person skilled in the art, preferably it is measured as described in the Materials and Methods' section under the topic ‘Thermal measurement and skin reddening’.

For instance, the sO₂ (oxygen saturation of haemoglobin) within the skin, the rHb (relative haemoglobin amount) within the skin or the temperature on the skin or the combination of any two or all three of them may be a measure of the accumulation of PBMCs. A higher value for the sO₂, rHb and/or the temperature indicates an accumulation of PBMCs. Additionally, in combination with the sO₂, the rHb and/or the temperature any or both of the blood flow parameters rFlow (relative blood flow) within the skin and Vel (blood flow velocity) within the skin may be increased to indicate an accumulation of PBMCs.

Hence, preferably, in any of the embodiments described herein and particularly in step (A-0), the accumulation of PBMCs is determined in terms of the sO₂ and/or the rHb, more preferably the sO₂, the rHb and/or the temperature. Even more preferably, the accumulation of PBMCs is determined in terms of the sO₂, the rHb and the temperature. Still even more preferably, the accumulation of PBMCs is determined in terms of the sO₂, the rHb, the temperature, the rFlow and the Vel.

Preferably, in any of the embodiments described herein and particularly in step (A-0), the sO₂, the rHb, the rFlow and/or the Vel within the skin may be measured by any suitable method known to the person skilled in the art, preferably they are measured as described in the Materials and Methods' section under the topic ‘O2C—Oxygen to See’.

Preferably, in any of the embodiments described herein and particularly in step (A-0), the temperature on the skin may be measured by any suitable method known to the person skilled in the art, preferably it is measured as described in the Materials and Methods' section under the topic ‘Thermal measurement and skin reddening’.

When the accumulation of PBMCs within the skin is determined in terms of the sO₂ within the skin, the increase of the sO₂ within the skin is preferably by the same amount as mentioned in % (percent) or %-points (percent points) in any of the embodiments of the present invention described under the outline note ‘As further regards step (A-3) for any of the embodiments as described herein’. If not mentioned otherwise, it is to be understood that any embodiment or definition described herein for the sO₂ and the increase of the sO₂ in % or %-points, particularly in any of the embodiments of step (A-3), is independently and mutatis mutandis applicable to the sO₂ and the increase of the sO₂ in any of the embodiments of step (A-0) described herein.

When the accumulation of PBMCs within the skin is determined in terms of the rHb within the skin, the increase of the rHb within the skin is preferably by the same amount as mentioned in % (percent) or AU (arbitrary units) in any of the embodiments of the present invention described under the outline note ‘As further regards step (A-3) for any of the embodiments as described herein’. If not mentioned otherwise, it is to be understood that any embodiment or definition described herein for the rHb and the increase of the rHb in % or AU, particularly in any of the embodiments of step (A-3), is independently and mutatis mutandis applicable to the rHb and the increase of the rHb in any of the embodiments of step (A-0) described herein.

When the accumulation of PBMCs within the skin is determined in terms of the rFlow within the skin, the increase of the rFlow within the skin is preferably by the same amount as mentioned in % or AU in any of the embodiments of the present invention described under the outline note ‘As further regards step (A-3) for any of the embodiments as described herein’. If not mentioned otherwise, it is to be understood that any embodiment or definition described herein for the rFlow and the increase of the rFlow in % or AU, particularly in any of the embodiments of step (A-3), is independently and mutatis mutandis applicable to the rFlow and the increase of the rFlow in any of the embodiments of step (A-0) described herein.

When the accumulation of PBMCs within the skin is determined in terms of the Vel within the skin, the increase of the Vel within the skin is preferably by the same amount as mentioned in % or AU in any of the embodiments of the present invention described under the outline note ‘As further regards step (A-3) for any of the embodiments as described herein’. If not mentioned otherwise, it is to be understood that any embodiment or definition described herein for the Vel and the increase of the Vel in % or AU, particularly in any of the embodiments of step (A-3), is independently and mutatis mutandis applicable to the Vel and the increase of the Vel in any of the embodiments of step (A-0) described herein.

When the accumulation of PBMCs within the skin is determined in terms of the temperature on the skin, the increase of the temperature on the skin is preferably by the same amount as mentioned in % (percent) or ° C. (degrees Celius) in any of the embodiments of the present invention described under the outline note ‘As further regards step (A-4) for any of the embodiments as described herein’. If not mentioned otherwise, it is to be understood that any embodiment or definition described herein for the temperature on the skin and the increase of the temperature on the skin in % or ° C., particularly in any of the embodiments of step (A-4), is independently and mutatis mutandis applicable to the temperature on the skin and the increase of the temperature on the skin in any of the embodiments of step (A-0) described herein.

Preferably, in any of the embodiments described herein and particularly in step (A-0), the accumulation of PBMCs may be to any extent and/or duration, more preferably, as long as a sufficient increase in the amount of PBMCs, even more preferably naïve lymphocytes, still more preferably naïve B-cells and/or naïve T-cells, still even more preferably naïve T-cells is achieved. Furthermore, there is preferably no upper limit for the extend and/or duration of the accumulation of PBMCs as long as no lesions and/or damaging of skin and/or capillaries of the skin is caused.

Preferably, in any of the embodiments described herein and particularly in step (A-0), the accumulation of PBMCs is indicated with reference to the accumulation of PBMCs or amount of PBMCs of untreated skin. More preferably, the skin in step (A-0) is of a skin area, preferably of a skin area [a], and the untreated skin is of an untreated skin area, wherein the untreated skin area is distanced apart from the skin area by 1 cm or more and 10 cm or less. The distance is preferably measured from a point on the outline of the skin area to a point on the outline of the untreated skin area, wherein the points with the smallest distance to each other are taken. More preferably, the untreated skin is the skin before the method of the present invention is carried out on the skin. Preferably, it is the skin of an arm or leg, more preferably of a human.

Typically, untreated skin has accumulation of PBMCs in terms of the sO₂ of 85% or less, more typically in the range of 30% or more and 85% or less, even more typically, 55% or more and 80% or less, preferably when the sO₂ is measured as described in the Materials and Methods' section under the topic ‘O2C—Oxygen to See’.

Typically, untreated skin has accumulation of PBMCs in terms of the rHb of 90 AU or less, more typically in the range of 30 AU or more and 90 AU or less, even more typically of 60 AU or more and 85 AU or less, preferably when the rHb is measured as described in the Materials and Methods' section under the topic ‘O2C—Oxygen to See’.

Typically, untreated skin has accumulation of PBMCs in terms of the rFlow of 110 AU or less, more typically in the range of 5 AU or more and 110 AU or less, even more typically of 10 AU or more and 80 AU or less, preferably when the rFlow is measured as described in the Materials and Methods' section under the topic ‘O2C—Oxygen to See’.

Typically, untreated skin has accumulation of PBMCs in terms of the Vel of 20 AU or less, more typically in the range of 5 AU or more and 20 AU or less, even more typically of 10 AU or more and 18 AU or less, preferably when the Vel is measured as described in the Materials and Methods' section under the topic ‘O2C—Oxygen to See’.

Typically, untreated skin has an accumulation of PBMCs in terms of the temperature, preferably the skin surface temperature, of 36° C. or less, more typically in the range of 20° C. or more and 36° C. or less, even more typically 22° C. or more and 34° C. or less, still more typically 24° C. or more and 34° C. or less, still even more typically 24° C. or more and 32° C. or less, preferably when the temperature on the skin is measured as described in the Materials and Methods' section ‘Thermal measurement and skin reddening’.

Preferably, in any of the embodiments described herein and particularly in step (A-0), the accumulation of PBMCs is established within 3 hours or less, even more preferably 1.5 hours or less, still more preferably 1 hour or less, still even more preferably 0.75 hours or less, further preferably 0.5 hours or less. Preferably the accumulation of PBMCs is established immediately up to 3 hours, more preferably 0.05 sec up to 1.5 hours, even more preferably 0.5 sec up to 1.5 hours, still more preferably 5 sec up to 1 hour, still even more preferably 10 sec up to 0.75 hours, further preferably 10 sec up to 0.5 hours, even further preferably 10 sec up to 0.25 hours.

Preferably, in any of the embodiments described herein and particularly in step (A-0), the accumulation of PBMCs is present, sustains and/or is maintained for a duration of time of 2 min or more, more preferably 10 min or more, even more preferably for 0.25 hours or more. There is no upper limit for the persistence of the accumulation of PBMCs, however, usually it is for a duration of time of 48 hours or less, more preferably 6 hours or less, even more preferably 3 hours or less, still more preferably 1.5 hour or less, still even more preferably 1 hour or less, further preferably 0.75 hours or less. Preferably the accumulation of PBMCs is present, sustains and/or is maintained for a duration of time in the range of 10 min or more and 48 hours or less, more preferably 0.25 hours or more and 48 hours or less, even more preferably 0.25 hours or more and 6 hours or less, still more preferably 0.5 hours or more and 3 hours or less, still even more preferably 0.75 hours or more and 1.5 hours or less.

The duration of time may be continuous or with interruptions, i.e. distributed to two or more sub-durations. Therefore, the duration of time is preferably the total duration of time which may be composed of a single continuous duration or the sum of two or more sub-durations.

Furthermore, the duration or total duration of time for which the accumulation of PBMCs is present and/or is maintained after performing any or all of steps (B), (B₁) and/or (C). Such the duration of time is for instance from at the same time when performing or immediately after performing any or all of steps (B), (B₁) and/or (C) to 15 hours or less, preferably 10 hours or less, more preferably 8 hours or less, even more preferably 6 hours or less, even more preferably 2 hours or less. Hence, the accumulation of PBMCs may for instance be present and/or maintained for the total duration of time by performing step (A-0) multiple times, e.g. by applying a heat pad several times to the skin or using a continuously warming heat patch. However, it is noted that this does not exclude that step (A-0) is performed before any or all of steps (B), (B₁) and/or (C) are performed.

Preferably, in any of the embodiments described herein and particularly in step (A-0), the generating of the accumulation of PBMCs may be, preferably, is accomplished by any means, method, substance(s) and/or procedure, wherein the means, method, substance(s) and/or procedure are not particularly limited as long as it is suitable and/or sufficient to generate an accumulation of PBMCs, preferably lymphocytes, within the skin and/or without damaging the skin like burning, causing lesions, rupture of capillaries and/or reflexive closure of the capillaries.

Preferably, in any of the embodiments described herein and particularly in step (A-0), the accumulation of PBMCs is generated by:
(A-1) generating a vasodilation of the capillaries within the skin of the subject, wherein it is to be understood that preferably any embodiment or definition thereof which is described under the outline note ‘As further regards step (A-1) for any of the embodiments as described herein’ is independently and mutatis mutandis applicable; and/or
(A-2) generating an increased blood volume within the skin of the subject, wherein it is to be understood that preferably any embodiment or definition thereof which is described under the outline note ‘As further regards step (A-2) for any of the embodiments as described herein’ is independently and mutatis mutandis applicable; and/or
(A-3) generating an increased sO₂ and/or an increased rHb within the skin, wherein it is to be understood that preferably any embodiment or definition thereof which is described under the outline note ‘As further regards step (A-3) for any of the embodiments as described herein’ is independently and mutatis mutandis applicable; and/or
(A-4) generating an increased temperature on the skin of the subject, wherein it is to be understood that preferably any embodiment or definition thereof which is described under the outline note ‘As further regards step (A-4) for any of the embodiments as described herein’ is independently and mutatis mutandis applicable; and/or
(A-5) generating a redness on the skin of the subject, wherein it is to be understood that preferably any embodiment or definition thereof which is described under the outline note ‘As further regards step (A-5) for any of the embodiments as described herein’ is independently and mutatis mutandis applicable; and/or
(A-6) administering conditioning energy to the skin of the subject, wherein it is to be understood that preferably any embodiment or definition thereof which is described under the outline note ‘As further regards step (A-6) for any of the embodiments as described herein’ is independently and mutatis mutandis applicable; and/or
(A-7) administering a skin-conditioning agent to the skin of the subject, wherein it is to be understood that preferably any embodiment or definition thereof which is described under the outline note ‘As further regards. step (A-7) for any of the embodiments as described herein’ is independently and mutatis mutandis applicable; and/or
(A-8) administering PBMCs to the skin the skin of the subject, preferably into the skin of the skin area, wherein it is to be understood that preferably any embodiment or definition thereof which is described under the outline note ‘As further regards. step (A-8) for any of the embodiments as described herein’ is independently and mutatis mutandis applicable.

Preferably, steps (A-1) to (A-8) may not be mutually exclusive.

Preferably, in step (A-0) in any of the embodiments described herein, the skin is of a skin area.

Preferably, in step (A-0) in any of the embodiments described herein, the accumulation of PBMCs is generated within the skin of the skin area.

More preferably, the accumulation of PBMCs is generated within the skin of the skin area and the conditioning energy, skin-conditioning agent and/or PBMCs is administered to the skin of the skin area.

Even more preferably, the accumulation of PBMCs is generated within the skin of the skin area, wherein the conditioning energy, skin-conditioning agent and/or PBMCs is administered to the skin of the skin area by applying the conditioning energy, skin-conditioning agent and/or PBMCs is to the skin of the application area.

Preferably, in step (A-0) in any of the embodiments described herein, the application area is that of the respective skin area.

More preferably, the skin area is the skin area [a] and/or the application area is the application area [a].

Preferably, in step (A-0) in any of the embodiments described herein, the skin area and the skin area [a] are the same skin area and skin area [a], respectively, as mentioned in any of the embodiments described herein. If not mentioned otherwise, it is to be understood that any embodiment or definition described herein in terms of step (A) and a skin area or skin area [a] is independently and mutatis mutandis applicable to the skin area and the skin area [a], respectively, of step (A-0).

Preferably, in step (A-0) in any of the embodiments described herein, the application area and the application area [a] are the same application area and application area [a], respectively, as mentioned in any of the embodiments described herein. If not mentioned otherwise, it is to be understood that any embodiment or definition described herein in terms of step (A) and an application area or application area [a] is independently and mutatis mutandis applicable to the application area and the application area [a], respectively, of step (A-0).

It is to be understood that any of the embodiments mentioned under the outline note ‘As further regards step (A-0) for any of the embodiments as described herein’ is independently applicable and/or combinable with any of the embodiments for the method as described herein and particularly with any embodiment of step (A) of the method as described herein.

As further regards step (A-1) for any of the embodiments as described herein

As stated above, step (A-1) requires generating a vasodilation of the capillaries within the skin of the subject.

Preferably, in any of the embodiments described herein and particularly in step (A-1), the vasodilation is of the capillaries within a sub-topical layer of the skin, more preferably within the dermis and/or the subcutis of the skin, preferably of the skin of the skin area.

Preferably, in any of the embodiments described herein and particularly in step (A-1), the vasodilation is to an extent and/or a duration suitable to achieve the beneficial effects, is pharmaceutically acceptable and/or does not cause lesions and/or a damaging of skin like burning, causing lesions and/or rupture of capillaries of the capillaries.

Preferably, in any of the embodiments described herein and particularly in step (A-1), the vasodilation may to any extent and/or any duration as long as it is pharmaceutically acceptable and does not cause lesions and/or a damaging of skin like burning, causing lesions and/or rupture of capillaries.

Preferably, in any of the embodiments described herein and particularly in step (A-1),
the vasodilation is indicated by; and/or
the vasodilation is to generate, preferably the vasodilation is generated to an extent and/or a duration is suitable and/or sufficient to generate:
   an accumulation of PBMCs within the skin, wherein it is to be understood that preferably any embodiment or definition thereof which is described under the outline note ‘As further regards step (A-0) for any of the embodiments as described herein’ is independently and mutatis mutandis applicable; and/or
   an increased blood volume within the skin, wherein it is to be understood that preferably any embodiment or definition thereof which is described under the outline note ‘As further regards step (A-2) for any of the embodiments as described herein’ is independently and mutatis mutandis applicable; and/or
   an increased sO₂ and/or an increased rHb within the skin, wherein it is to be understood that preferably any embodiment or definition thereof which is described under the outline note ‘As further regards step (A-3) for any of the embodiments as described herein’ is independently and mutatis mutandis applicable; and/or
   an increased temperature on the skin, wherein it is to be understood that preferably any embodiment or definition thereof which is described under the outline note ‘As further regards step (A-4) for any of the embodiments as described herein’ is independently and mutatis mutandis applicable; and/or
   a redness on the skin, wherein it is to be understood that preferably any embodiment or definition thereof which is described under the outline note ‘As further regards step (A-5) for any of the embodiments as described herein’ is independently and mutatis mutandis applicable.

Preferably, in any of the embodiments described herein and particularly in step (A-1), the vasodilation of the capillaries within the skin is measured by any suitable method known to the person skilled in the art, preferably it is measured as described in the Materials and Methods' section under the topic ‘Thermal measurement and skin reddening’.

For instance, the sO₂ (oxygen saturation of haemoglobin) within the skin, the rHb (relative haemoglobin amount) within the skin or the temperature on the skin or the combination of any two or all three of them may be a measure of the vasodilation. A higher value for the sO₂, rHb and/or the temperature on the skin indicates a more pronounced vasodilation. Additionally, in combination with the sO₂, the rHb and/or the temperature on the skin any or both of the blood flow parameters rFlow (relative blood flow) within the skin and Vel (blood flow velocity) within the skin may be increased to indicate a vasodilation.

Hence, preferably, in any of the embodiments described herein and particularly in step (A-1), the vasodilation is determined in terms of the sO₂, the rHb and/or the temperature on the skin. More preferably, the vasodilation is determined in terms of the sO₂, the rHb and the temperature on the skin. Even more preferably, the vasodilation is determined in terms of the sO₂, the rHb, the temperature on the skin, the rFlow and the Vel.

Preferably, in any of the embodiments described herein and particularly in step (A-1), the sO₂, the rHb, the rFlow and/or the Vel may be measured by any suitable method known to the person skilled in the art, preferably they are measured as described in the Materials and Methods' section under the topic ‘O2C—Oxygen to See’.

Preferably, in any of the embodiments described herein and particularly in step (A-1), the temperature on the skin may be measured by any suitable method known to the person skilled in the art, preferably it is measured as described in the Materials and Methods' section under the topic ‘Thermal measurement and skin reddening’.

When the vasodilation within the skin is determined in terms of the sO₂ within the skin, the increase of the sO₂ within the skin is preferably by the same amount as mentioned in % (percent) or %-points (percent points) in any of the embodiments of the present invention described under the outline note ‘As further regards step (A-3) for any of the embodiments as described herein’. If not mentioned otherwise, it is to be understood that any embodiment or definition described herein for the sO₂ and the increase of the sO₂ in % or %-points, particularly in any of the embodiments of step (A-3), is independently and mutatis mutandis applicable to the sO₂ and the increase of the sO₂ in any of the embodiments of step (A-1) described herein.

When the vasodilation within the skin is determined in terms of the rHb within the skin, the increase of the rHb within the skin is preferably by the same amount as mentioned in % (percent) or AU (arbitrary units) in any of the embodiments of the present invention described under the outline note ‘As further regards step (A-3) for any of the embodiments as described herein’. If not mentioned otherwise, it is to be understood that any embodiment or definition described herein for the rHb and the increase of the rHb in % or AU, particularly in any of the embodiments of step (A-3), is independently and mutatis mutandis applicable to the rHb and the increase of the rHb in any of the embodiments of step (A-1) described herein.

When the vasodilation within the skin is determined in terms of the rFlow within the skin, the increase of the rFlow within the skin is preferably by the same amount as mentioned in % or AU in any of the embodiments of the present invention described under the outline note ‘As further regards step (A-3) for any of the embodiments as described herein’. If not mentioned otherwise, it is to be understood that any embodiment or definition described herein for the rFlow and the increase of the rFlow in % or AU, particularly in any of the embodiments of step (A-3), is independently and mutatis mutandis applicable to the rFlow and the increase of the rFlow in any of the embodiments of step (A-1) described herein.

When the vasodilation within the skin is determined in terms of the Vel within the skin, the increase of the Vel within the skin is preferably by the same amount as mentioned in % or AU in any of the embodiments of the present invention described under the outline note ‘As further regards step (A-3) for any of the embodiments as described herein’. If not mentioned otherwise, it is to be understood that any embodiment or definition described herein for the Vel and the increase of the Vel in % or AU, particularly in any of the embodiments of step (A-3), is independently and mutatis mutandis applicable to the Vel and the increase of the Vel in any of the embodiments of step (A-1) described herein.

When the vasodilation within the skin is determined in terms of the temperature on the skin, the increase of the temperature on the skin is preferably by the same amount as mentioned in % (percent) or ° C. (degrees Celius) in any of the embodiments of the present invention described under the outline note ‘As further regards step (A-4) for any of the embodiments as described herein’. If not mentioned otherwise, it is to be understood that any embodiment or definition described herein for the temperature on the skin and the increase of the temperature on the skin in % or ° C., particularly in any of the embodiments of step (A-4), is independently and mutatis mutandis applicable to the temperature on the skin and the increase of the temperature on the skin in any of the embodiments of step (A-1) described herein.

Preferably, in any of the embodiments described herein and particularly in step (A-1), the vasodilation may be to any extent and/or duration, more preferably, as long as a sufficient vasodilation is achieved. Furthermore, there is preferably no upper limit for the extend and/or duration of the vasodilation as long as no lesions and/or damaging of skin and/or capillaries of the skin is caused.

Preferably, in any of the embodiments described herein and particularly in step (A-1), the vasodilation is indicated with reference to the vasodilation of untreated skin. More preferably, the skin in step (A-1) is of a skin area, preferably of a skin area [a], and the untreated skin is of an untreated skin area, wherein the untreated skin area is distanced apart from the skin area by 1 cm or more and 10 cm or less. The distance is preferably measured from a point on the outline of the skin area to a point on the outline of the untreated skin area, wherein the points with the smallest distance to each other are taken. More preferably, the untreated skin is the skin before the method of the present invention is carried out on the skin. Preferably, it is the skin of an arm or leg, more preferably of a human.

Typically, untreated skin has a vasodilation in terms of the sO₂ of 85% or less, more typically in the range of 30% or more and 85% or less, even more typically, 55% or more and 80% or less, preferably when the sO₂ is measured as described in the Materials and Methods' section under the topic ‘O2C—Oxygen to See’.

Typically, untreated skin has a vasodilation in terms of the rHb of 90 AU or less, more typically in the range of 30 AU or more and 90 AU or less, even more typically of 60 AU or more and 85 AU or less, preferably when the rHb is measured as described in the Materials and Methods' section under the topic ‘O2C—Oxygen to See’.

Typically, untreated skin has a vasodilation in terms of the rFlow of 110 AU or less, more typically in the range of 5 AU or more and 110 AU or less, even more typically of 10 AU or more and 80 AU or less, preferably when the rFlow is measured as described in the Materials and Methods' section under the topic ‘O2C—Oxygen to See’.

Typically, untreated skin has a vasodilation in terms of the Vel of 20 AU or less, more typically in the range of 5 AU or more and AU or less, even more typically of 10 AU or more and 18 AU or less, preferably when the Vel is measured as described in the Materials and Methods' section under the topic ‘O2C—Oxygen to See’.

Typically, untreated skin has a vasodilation in terms of the temperature, preferably the skin surface temperature, of 36° C. or less, more typically in the range of 20° C. or more and 36° C. or less, even more typically 22° C. or more and 34° C. or less, still more typically 24° C. or more and 34° C. or less, still even more typically 24° C. or more and 32° C. or less, preferably when the temperature on the skin is measured as described in the Materials and Methods' section ‘Thermal measurement and skin reddening’.

Preferably, in any of the embodiments described herein and particularly in step (A-1), the vasodilation is established within 3 hours or less, even more preferably 1.5 hours or less, still more preferably 1 hour or less, still even more preferably 0.75 hours or less, further preferably 0.5 hours or less. Preferably the vasodilation is established immediately up to 3 hours, more preferably 0.05 sec up to 1.5 hours, even more preferably 0.5 sec up to 1.5 hours, still more preferably 5 sec up to 1 hour, still even more preferably 10 sec up to 0.75 hours, further preferably 10 sec up to 0.5 hours, even further preferably 10 sec up to 0.25 hours.

Preferably, in any of the embodiments described herein and particularly in step (A-1), the vasodilation is present, sustains and/or is maintained for a duration of time of 2 min or more, more preferably 10 min or more, even more preferably for 0.25 hours or more. There is no upper limit for the persistence of the vasodilation, however, usually it is for a duration of time of 6 hours or less, more preferably 3 hours or less, even more preferably 1.5 hour or less, still more preferably 1 hour or less, still even more preferably 0.75 hours or less. Preferably the vasodilation is present, sustains and/or is maintained for a duration of time in the range of 2 min or more and 6 hours or less, more preferably 5 min or more and 3 hours or less, even more preferably 10 min or more and 1.5 hour or less, still more preferably 0.25 hours or more and 1 hour or less, still even more preferably 0.25 hours or more and 0.75 hours or less.

The duration of time may be continuous or with interruptions, i.e. distributed to two or more sub-durations. Therefore, the duration of time is preferably the total duration of time which may be composed of a single continuous duration or the sum of two or more sub-durations.

Furthermore, the duration or total duration of time for which the vasodilation is present and/or is maintained after performing any or all of steps (B), (B₁) and/or (C). Such the duration of time is for instance from at the same time when performing or immediately after performing any or all of steps (B), (B₁) and/or (C) to 15 hours or less, preferably 10 hours or less, more preferably 8 hours or less, even more preferably 6 hours or less, even more preferably 2 hours or less. Hence, the vasodilation may for instance be present and/or maintained for the total duration of time by performing step (A-1) multiple times, e.g. by applying a heat pad several times to the skin or using a continuously warming heat patch. However, it is noted that this does not exclude that step (A-1) is performed before any or all of steps (B), (B₁) and/or (C) are performed.

Preferably, in any of the embodiments described herein and particularly in step (A-1), the generating of the vasodilation of the capillaries may be, preferably is accomplished by any means, method(s), substance(s) and/or procedure(s) as long as it is suitable and/or sufficient to generate a vasodilation of the capillaries within the skin and/or without damaging the skin like burning and/or causing lesions rupture of capillaries.

Preferably, in any of the embodiments described herein and particularly in step (A-1), the generating of the vasodilation of the capillaries is generated by:
(A-0) generating an accumulation of PBMCs within the skin, wherein it is to be understood that preferably any embodiment or definition thereof which is described under the outline note ‘As further regards step (A-0) for any of the embodiments as described herein’ is independently and mutatis mutandis applicable; and/or
(A-2) generating an increased blood volume within the skin, wherein it is to be understood that preferably any embodiment or definition thereof which is described under the outline note ‘As further regards step (A-2) for any of the embodiments as described herein’ is independently and mutatis mutandis applicable; and/or
(A-3) generating an increased sO₂ and/or an increased rHb within the skin, wherein it is to be understood that preferably any embodiment or definition thereof which is described under the outline note ‘As further regards step (A-3) for any of the embodiments as described herein’ is independently and mutatis mutandis applicable; and/or
(A-4) generating an increased temperature on the skin, wherein it is to be understood that preferably any embodiment or definition thereof which is described under the outline note ‘As further regards step (A-4) for any of the embodiments as described herein’ is independently and mutatis mutandis applicable; and/or
(A-5) generating a redness on the skin, wherein it is to be understood that preferably any embodiment or definition thereof which is described under the outline note ‘As further regards step (A-5) for any of the embodiments as described herein’ is independently and mutatis mutandis applicable; and/or
(A-6) administering conditioning energy to the skin, wherein it is to be understood that preferably any embodiment or definition thereof which is described under the outline note ‘As further regards step (A-6) for any of the embodiments as described herein’ is independently and mutatis mutandis applicable; and/or
(A-7) administering a skin-conditioning agent to the skin, wherein it is to be understood that preferably any embodiment or definition thereof which is described under the outline note ‘As further regards step (A-7) for any of the embodiments as described herein’ is independently and mutatis mutandis applicable.

Preferably, steps (A-0) and (A-2) to (A-7) may not be mutually exclusive.

Preferably, in step (A-1) in any of the embodiments described herein, the skin is of a skin area.

Preferably, in step (A-1) in any of the embodiments described herein, the vasodilation is generated within the skin of the skin area.

More preferably, the vasodilation is generated within the skin of the skin area and the conditioning energy and/or skin-conditioning agent is administered to the skin of the skin area.

Even more preferably, the vasodilation is generated within the skin of the skin area, wherein the conditioning energy and/or skin-conditioning agent is administered to the skin of the skin area by applying the conditioning energy and/or skin-conditioning agent to the skin of the application area.

Preferably, in step (A-1) in any of the embodiments described herein, the application area is that of the respective skin area.

More preferably, the skin area is the skin area [a] and/or the application area is the application area [a].

Preferably, in step (A-1) in any of the embodiments described herein, the skin area and the skin area [a] are the same skin area and skin area [a], respectively, as mentioned in any of the embodiments described herein. If not mentioned otherwise, it is to be understood that any embodiment or definition described herein in terms of step (A) and a skin area or skin area [a] is independently and mutatis mutandis applicable to the skin area and the skin area [a], respectively, of step (A-1).

Preferably, in step (A-1) in any of the embodiments described herein, the application area and the application area [a] are the same application area and application area [a], respectively, as mentioned in any of the embodiments described herein. If not mentioned otherwise, it is to be understood that any embodiment or definition described herein in terms of step (A) and an application area or application area [a] is independently and mutatis mutandis applicable to the application area and the application area [a], respectively, of step (A-1).

It is to be understood that any of the embodiments mentioned under the outline note ‘As further regards step (A-1) for any of the embodiments as described herein’ is independently applicable and/or combinable with any of the embodiments for the method as described herein and particularly with any embodiment of step (A) of the method as described herein.

As further regards step (A-2) for any of the embodiments as described herein

As stated above, step (A-2) requires generating an increased blood volume within the skin of the subject, preferably of the skin area.

Preferably, in any of the embodiments described herein and particularly in step (A-2), the blood volume is increased in a sub-topical layer, more preferably the dermis and/or subcutis. More preferably, the blood volume is increased in a sub-topical layer of the skin, more preferably the dermis and/or subcutis of the skin, preferably the skin of the skin area. Preferably, in any of the embodiments described herein and particularly in step (A-2), the blood volume is increased within the lumen of the capillaries, preferably the capillaries of the skin, more preferably the capillaries of the skin area.

More preferably, in any of the embodiments described herein and particularly in step (A-2), the blood volume is increased within the lumen of the capillaries of a sub-topical layer of the skin, even more preferably the capillaries of the dermis and/or the subcutis of the skin, preferably the skin of the skin area.

Preferably, in any of the embodiments described herein and particularly in step (A-2), the blood volume is increased to any extent and/or duration suitable to achieve the beneficial effects, is pharmaceutically acceptable and/or does not cause lesions and/or a damaging of skin like burning, causing lesions, rupture of capillaries and/or reflexive closure of the capillaries.

Preferably, in any of the embodiments described herein and particularly in step (A-2), the blood volume may be increased to any extent and/or any duration as long as it is pharmaceutically acceptable and does not cause lesions and/or a damaging of skin like burning, causing lesions, rupture of capillaries and/or reflexive closure of the capillaries.

Preferably, in any of the embodiments described herein and particularly in step (A-2),
the increased blood volume is indicated by; and/or
the blood volume is increased to generate, preferably the blood volume is increased to an extent and/or a duration is suitable and/or sufficient to generate:
   an accumulation of PBMCs within the skin, wherein it is to be understood that preferably any embodiment or definition thereof which is described under the outline note ‘As further regards step (A-0) for any of the embodiments as described herein’ is independently and mutatis mutandis applicable; and/or
   a vasodilation of the capillaries within the skin, wherein it is to be understood that preferably any embodiment or definition thereof which is described under the outline note ‘As further regards. step (A-1) for any of the embodiments as described herein’ is independently and mutatis mutandis applicable; and/or
   an increased sO₂ and/or an increased rHb within the skin, wherein it is to be understood that preferably any embodiment or definition thereof which is described under the outline note ‘As further regards step (A-3) for any of the embodiments as described herein’ is independently and mutatis mutandis applicable; and/or
   an increased temperature on the skin, wherein it is to be understood that preferably any embodiment or definition thereof which is described under the outline note ‘As further regards step (A-4) for any of the embodiments as described herein’ is independently and mutatis mutandis applicable; and/or
   a redness on the skin, wherein it is to be understood that preferably any embodiment or definition thereof which is described under the outline note ‘As further regards step (A-5) for any of the embodiments as described herein’ is independently and mutatis mutandis applicable.

Preferably, in any of the embodiments described herein and particularly in step (A-2), the increased blood volume of the capillaries within the skin is measured by any suitable method known to the person skilled in the art, preferably it is measured as described in the Materials and Methods' section under the topic ‘Thermal measurement and skin reddening’.

For instance, the sO₂ (oxygen saturation of haemoglobin) within the skin, the rHb (relative haemoglobin amount) within the skin or the temperature on the skin or the combination of any two or all three of them may be a measure of the blood volume. A higher value for the sO₂, rHb and/or the temperature on the skin indicates an increased blood volume. Additionally, in combination with the sO₂, the rHb and/or the temperature on the skin any or both of the blood flow parameters rFlow (relative blood flow) and Vel (blood flow velocity) may be increased to indicate an increased blood volume.

Hence, preferably, in any of the embodiments described herein and particularly in step (A-2), the increased blood volume is determined in terms of the sO₂, the rHb and/or the temperature on the skin. More preferably, the increased blood volume is determined in terms of the sO₂, the rHb and the temperature on the skin. Even more preferably, the increased blood volume is determined in terms of the sO₂, the rHb, the temperature on the skin, the rFlow and the Vel.

Preferably, in any of the embodiments described herein and particularly in step (A-2), the sO₂, the rHb, the rFlow and/or the Vel may be measured by any suitable method known to the person skilled in the art, preferably they are measured as described in the Materials and Methods' section under the topic ‘O2C—Oxygen to See’.

Preferably, in any of the embodiments described herein and particularly in step (A-2), the temperature on the skin may be measured by any suitable method known to the person skilled in the art, preferably it is measured as described in the Materials and Methods' section under the topic ‘Thermal measurement and skin reddening’.

When the increased blood volume within the skin is determined in terms of the sO₂ within the skin, the increase of the sO₂ within the skin is preferably by the same amount as mentioned in % (percent) or %-points (percent points) in any of the embodiments of the present invention described under the outline note ‘As further regards step (A-3) for any of the embodiments as described herein’. If not mentioned otherwise, it is to be understood that any embodiment or definition described herein for the sO₂ and the increase of the sO₂ in % or %-points, particularly in any of the embodiments of step (A-3), is independently and mutatis mutandis applicable to the sO₂ and the increase of the sO₂ in any of the embodiments of step (A-2) described herein.

When the increased blood volume within the skin is determined in terms of the rHb within the skin, the increase of the rHb within the skin is preferably by the same amount as mentioned in % (percent) or AU (arbitrary units) in any of the embodiments of the present invention described under the outline note ‘As further regards step (A-3) for any of the embodiments as described herein’. If not mentioned otherwise, it is to be understood that any embodiment or definition described herein for the rHb and the increase of the rHb in % or AU, particularly in any of the embodiments of step (A-3), is independently and mutatis mutandis applicable to the rHb and the increase of the rHb in any of the embodiments of step (A-2) described herein.

When the increased blood volume within the skin is determined in terms of the rFlow within the skin, the increase of the rFlow within the skin is preferably by the same amount as mentioned in % or AU in any of the embodiments of the present invention described under the outline note ‘As further regards step (A-3) for any of the embodiments as described herein’. If not mentioned otherwise, it is to be understood that any embodiment or definition described herein for the rFlow and the increase of the rFlow in % or AU, particularly in any of the embodiments of step (A-3), is independently and mutatis mutandis applicable to the rFlow and the increase of the rFlow in any of the embodiments of step (A-2) described herein.

When the increased blood volume within the skin is determined in terms of the Vel within the skin, the increase of the Vel within the skin is preferably by the same amount as mentioned in % or AU in any of the embodiments of the present invention described under the outline note ‘As further regards step (A-3) for any of the embodiments as described herein’. If not mentioned otherwise, it is to be understood that any embodiment or definition described herein for the Vel and the increase of the Vel in % or AU, particularly in any of the embodiments of step (A-3), is independently and mutatis mutandis applicable to the Vel and the increase of the Vel in any of the embodiments of step (A-2) described herein.

When the increased blood volume within the skin is determined in terms of the temperature on the skin, the increase of the temperature on the skin is preferably by the same amount as mentioned in % (percent) or ° C. (degrees Celius) in any of the embodiments of the present invention described under the outline note ‘As further regards step (A-4) for any of the embodiments as described herein’. If not mentioned otherwise, it is to be understood that any embodiment or definition described herein for the temperature on the skin and the increase of the temperature on the skin in % or ° C., particularly in any of the embodiments of step (A-4), is independently and mutatis mutandis applicable to the temperature on the skin and the increase of the temperature on the skin in any of the embodiments of step (A-2) described herein.

Preferably, in any of the embodiments described herein and particularly in step (A-2), the increase in the blood volume may be to any extent and/or duration, more preferably, as long as a sufficient increase in the blood volume is achieved. Furthermore, there is preferably no upper limit for the extend of the increase and/or duration of the blood volume as long as no lesions and/or damaging of skin and/or capillaries of the skin is caused.

Preferably, in any of the embodiments described herein and particularly in step (A-2), the increased blood volume is indicated with reference to the blood volume of untreated skin. More preferably, the skin in step (A-2) is of a skin area, preferably of a skin area [a], and the untreated skin is of an untreated skin area, wherein the untreated skin area is distanced apart from the skin area by 1 cm or more and 10 cm or less. The distance is preferably measured from a point on the outline of the skin area to a point on the outline of the untreated skin area, wherein the points with the smallest distance to each other are taken. More preferably, the untreated skin is the skin before the method of the present invention is carried out on the skin. Preferably, it is the skin of an arm or leg, more preferably of a human.

Typically, untreated skin has a blood volume in terms of the sO₂ of 85% or less, more typically in the range of 30% or more and 85% or less, even more typically, 55% or more and 80% or less, preferably when the sO₂ is measured as described in the Materials and Methods' section under the topic ‘O2C—Oxygen to See’.

Typically, untreated skin has a blood volume in terms of the rHb of 90 AU or less, more typically in the range of 30 AU or more and 90 AU or less, even more typically of 60 AU or more and 85 AU or less, preferably when the rHb is measured as described in the Materials and Methods' section under the topic ‘O2C—Oxygen to See’.

Typically, untreated skin has a blood volume in terms of the rFlow of 110 AU or less, more typically in the range of 5 AU or more and 110 AU or less, even more typically of 10 AU or more and 80 AU or less, preferably when the rFlow is measured as described in the Materials and Methods' section under the topic ‘O2C—Oxygen to See’.

Typically, untreated skin has a blood volume in terms of the Vel of 20 AU or less, more typically in the range of 5 AU or more and AU or less, even more typically of 10 AU or more and 18 AU or less, preferably when the Vel is measured as described in the Materials and Methods' section under the topic ‘O2C—Oxygen to See’.

Typically, untreated skin has a blood volume in terms of the temperature, preferably the skin surface temperature, of 36° C. or less, more typically in the range of 20° C. or more and 36° C. or less, even more typically 22° C. or more and 34° C. or less, still more typically 24° C. or more and 34° C. or less, still even more typically 24° C. or more and 32° C. or less, preferably when the temperature on the skin is measured as described in the Materials and Methods' section ‘Thermal measurement and skin reddening’.

Preferably, in any of the embodiments described herein and particularly in step (A-2), the increased blood volume is established within 3 hours or less, even more preferably 1.5 hours or less, still more preferably 1 hour or less, still even more preferably 0.75 hours or less, further preferably 0.5 hours or less. Preferably the increased blood volume is established immediately up to 3 hours, more preferably 0.05 sec up to 1.5 hours, even more preferably 0.5 sec up to 1.5 hours, still more preferably 5 sec up to 1 hour, still even more preferably 10 sec up to 0.75 hours, further preferably 10 sec up to 0.5 hours, even further preferably 10 sec up to 0.25 hours.

Preferably, in any of the embodiments described herein and particularly in step (A-2), the increased blood volume is present, sustains and/or is maintained for a duration of time of 2 min or more, more preferably 10 min or more, even more preferably for 0.25 hours or more. There is no upper limit for the persistence of the increased blood volume, however, usually it is for a duration of time of 6 hours or less, more preferably 3 hours or less, even more preferably 1.5 hour or less, still more preferably 1 hour or less, still even more preferably 0.75 hours or less. Preferably the increased blood volume is present, sustains and/or is maintained for a duration of time in the range of 2 min or more and 6 hours or less or less, more preferably 5 min or more and 3 hours or less, even more preferably 10 min or more and 1.5 hour or less or less, still more preferably 0.25 hours or more and 1 hour or less, still even more preferably 0.25 hours or more and 0.75 hours or less.

The duration of time may be continuous or with interruptions, i.e. distributed to two or more sub-durations. Therefore, the duration of time is preferably the total duration of time which may be composed of a single continuous duration or the sum of two or more sub-durations.

Furthermore, the duration or total duration of time for which the increased blood volume is present and/or is maintained after performing any or all of steps (B), (B₁) and/or (C). Such the duration of time is for instance from at the same time when performing or immediately after performing any or all of steps (B), (B₁) and/or (C) to 15 hours or less, preferably 10 hours or less, more preferably 8 hours or less, even more preferably 6 hours or less, even more preferably 2 hours or less. Hence, the increased blood volume may for instance be present and/or maintained for the total duration of time by performing step (A-2) multiple times, e.g. by applying a heat pad several times to the skin or using a continuously warming heat patch. However, it is noted that this does not exclude that step (A-2) is performed before any or all of steps (B), (B₁) and/or (C) are performed.

Preferably, in any of the embodiments described herein and particularly in step (A-2), the generating of the increased blood volume within the skin may be, preferably is accomplished by any means, method(s), substance(s) and/or procedure(s) as long as it is suitable and/or sufficient to generate an increased blood volume within the skin and/or without damaging the skin like burning, causing lesions, rupture of capillaries and/or reflexive closure of the capillaries.

Preferably, in any of the embodiments described herein and particularly in step (A-2), the generating of the increased blood volume is generated by:
(A-0) generating an accumulation of PBMCs within the skin, wherein it is to be understood that preferably any embodiment or definition thereof which is described under the outline note ‘As further regards step (A-0) for any of the embodiments as described herein’ is independently and mutatis mutandis applicable; and/or
(A-1) generating a vasodilation of the capillaries within the skin, wherein it is to be understood that preferably any embodiment or definition thereof which is described under the outline note ‘As further regards step (A-1) for any of the embodiments as described herein’ is independently and mutatis mutandis applicable; and/or
(A-3) generating an increased sO₂ and/or an increased rHb within the skin, wherein it is to be understood that preferably any embodiment or definition thereof which is described under the outline note ‘As further regards step (A-3) for any of the embodiments as described herein’ is independently and mutatis mutandis applicable; and/or
(A-4) generating an increased temperature on the skin, wherein it is to be understood that preferably any embodiment or definition thereof which is described under the outline note ‘As further regards step (A-4) for any of the embodiments as described herein’ is independently and mutatis mutandis applicable; and/or
(A-5) generating a redness on the skin, wherein it is to be understood that preferably any embodiment or definition thereof which is described under the outline note ‘As further regards step (A-5) for any of the embodiments as described herein’ is independently and mutatis mutandis applicable; and/or
(A-6) administering conditioning energy to the skin, wherein it is to be understood that preferably any embodiment or definition thereof which is described under the outline note ‘As further regards step (A-6) for any of the embodiments as described herein’ is independently and mutatis mutandis applicable; and/or
(A-7) administering a skin-conditioning agent to the skin, wherein it is to be understood that preferably any embodiment or definition thereof which is described under the outline note ‘As further regards step (A-7) for any of the embodiments as described herein’ is independently and mutatis mutandis applicable.

Preferably, steps (A-0), (A-1) and (A-3) to (A-7) may not be mutually exclusive.

Preferably, in step (A-2) in any of the embodiments described herein, the skin is of a skin area.

Preferably, in step (A-2) in any of the embodiments described herein, the increased blood volume is generated within the skin of the skin area.

More preferably, the increased blood volume is generated within the skin of the skin area and the conditioning energy and/or skin-conditioning agent is administered to the skin of the skin area.

Even more preferably, the increased blood volume is generated within the skin of the skin area, wherein the conditioning energy and/or skin-conditioning agent is administered to the skin of the skin area by applying the conditioning energy and/or skin-conditioning agent to the skin of the application area.

Preferably, in step (A-2) in any of the embodiments described herein, the application area is that of the respective skin area.

More preferably, the skin area is the skin area [a] and/or the application area is the application area [a].

Preferably, in step (A-2) in any of the embodiments described herein, the skin area and the skin area [a] are the same skin area and skin area [a], respectively, as mentioned in any of the embodiments described herein. If not mentioned otherwise, it is to be understood that any embodiment or definition described herein in terms of step (A) and a skin area or skin area [a] is independently and mutatis mutandis applicable to the skin area and the skin area [a], respectively, of step (A-2).

Preferably, in step (A-2) in any of the embodiments described herein, the application area and the application area [a] are the same application area and application area [a], respectively, as mentioned in any of the embodiments described herein. If not mentioned otherwise, it is to be understood that any embodiment or definition described herein in terms of step (A) and an application area or application area [a] is independently and mutatis mutandis applicable to the application area and the application area [a], respectively, of step (A-2).

It is to be understood that any of the embodiments mentioned under the outline note ‘As further regards step (A-2) for any of the embodiments as described herein’ is independently applicable and/or combinable with any of the embodiments for the method as described herein and particularly with any embodiment of step (A) of the method as described herein.

As further regards step (A-3) for any of the embodiments as described herein

As stated above, step (A-3) requires generating an increased sO₂ (oxygen saturation of haemoglobin) and/or an increased rHb (relative haemoglobin amount) within the skin of the subject.

The sO₂, the rHb or the combination of both are parameters of the postcapillary system in the skin and indicative for a condition of the capillary system within the skin.

Preferably, in any of the embodiments described herein and particularly in step (A-3), the increased sO₂ and/or the increased rHb is of the postcapillary system within a sub-topical layer of the skin, more preferably within the dermis and/or the subcutis of the skin, even more preferably within the dermis of the skin, preferably of the skin of the skin area.

Preferably, in any of the embodiments described herein and particularly in step (A-3), the increased sO₂ and/or the increased rHb is indicative for an increased sO₂ and/or the increased rHb of the capillaries within a sub-topical layer of the skin, more preferably within the dermis and/or the subcutis of the skin, even more preferably within the dermis of the skin, preferably of the skin of the skin area.

Preferably, step (A-3) requires generating in addition an increased rFlow (relative blood flow) and/or an increased Vel (blood flow velocity) within the skin.

Hence, step (A-3) preferably, requires generating an increased sO₂ and/or an increased rHb and an increased rFlow and/or an increased Vel within the skin.

More preferably, step (A-3) requires generating an increased sO₂, an increased rHb and in addition an increased rFlow and/or an increased Vel within the skin.

Even more preferably, step (A-3) requires generating an increased sO₂, an increased rHb, an increased rFlow and an increased Vel within the skin.

The rFlow or the Vel or the combination of both are also parameters of the postcapillary system in the skin and indicative for a condition of the capillary system within the skin.

Preferably, in any of the embodiments described herein and particularly in step (A-3), the increased rFlow and/or the increased Vel is of the postcapillary system within a sub-topical layer of the skin, more preferably within the dermis and/or the subcutis of the skin, even more preferably within the dermis of the skin, preferably of the skin of the skin area.

Preferably, in any of the embodiments described herein and particularly in step (A-3), the increased rFlow and/or the increased Vel is indicative for an increased rFlow and/or the increased Vel of the capillaries within a sub-topical layer of the skin, more preferably within the dermis and/or the subcutis of the skin, even more preferably within the dermis of the skin, preferably of the skin of the skin area.

Preferably, in any of the embodiments described herein and particularly in step (A-3), the sO₂, the rHb, the rFlow and/or the Vel is increased to any extent and/or duration suitable to achieve the beneficial effects, is pharmaceutically acceptable and/or does not cause lesions and/or a damaging of skin like burning, causing lesions, rupture of capillaries and/or reflexive closure of the capillaries.

Preferably, in any of the embodiments described herein and particularly in step (A-3), the sO₂, the rHb, the rFlow and/or the Vel may be increased to any extent and/or any duration as long as it is pharmaceutically acceptable and does not cause lesions and/or a damaging of skin like burning, causing lesions, rupture of capillaries and/or reflexive closure of the capillaries.

Preferably, in any of the embodiments of the present invention described herein and particularly in step (A-3),
the increased sO₂, the increased rHb, increased rFlow and/or increased Vel is indicated by; and/or
the increased sO₂, the increased rHb, increased rFlow and/or increased Vel is to generate, preferably the increased sO₂, the increased rHb, increased rFlow and/or increased Vel is generated to an extent and/or a duration is suitable and/or sufficient to generate:
   an accumulation of PBMCs within the skin, wherein it is to be understood that preferably any embodiment or definition thereof which is described under the outline note ‘As further regards step (A-0) for any of the embodiments as described herein’ is independently and mutatis mutandis applicable; and/or
   a vasodilation of the capillaries within the skin, wherein it is to be understood that preferably any embodiment or definition thereof which is described under the outline note ‘As further regards step (A-1) for any of the embodiments as described herein’ is independently and mutatis mutandis applicable; and/or
   an increased blood volume within the skin, wherein it is to be understood that preferably any embodiment or definition thereof which is described under the outline note ‘As further regards step (A-2) for any of the embodiments as described herein’ is independently and mutatis mutandis applicable; and/or
   an increased temperature on the skin, wherein it is to be understood that preferably any embodiment or definition thereof which is described under the outline note ‘As further regards step (A-4) for any of the embodiments as described herein’ is independently and mutatis mutandis applicable; and/or
   a redness on the skin, wherein it is to be understood that preferably any embodiment or definition thereof which is described under the outline note ‘As further regards step (A-5) for any of the embodiments as described herein’ is independently and mutatis mutandis applicable.

Preferably, in any of the embodiments described herein and particularly in step (A-3):
the sO₂ is increased by 2% or more and/or increased by 2%-points or more; and/or
the rHb is increased by 2% or more and/or by 2 AU (arbitrary units) or more; and/or
the rFlow is increased by 10% or more and/or by 20 AU (arbitrary units) or more; and/or
the Vel is increased by 10% or more and/or by 3 AU (arbitrary units) or more.

Preferably, in any of the embodiments described herein and particularly in step (A-3), the sO₂, the rHb, the rFlow and/or the Vel may be measured by any suitable method known to the person skilled in the art, preferably they are measured as described in the Materials and Methods' section under the topic ‘O2C—Oxygen to See’.

The sO₂ is increased:
more preferably, by 5% or more, even more preferably by 8% or more, still more preferably by 14% or more. There is no upper limit for the extend of the increase as long as no lesions and/or damaging of skin and/or capillaries of the skin is caused, however, usually, the sO₂ is increased by 200% or less, preferably 100% or less, preferably 50% or less. More preferably, the sO₂ is increased in the range of 2% or more and 200% or less, even more preferably 5% or more and 100% or less, still more preferably 8% or more and 50% or less, still even more preferably 14% or more and 50% or less; and/or
more preferably, by 4%-points or more, even more preferably by 6%-points or more, still more preferably by 9%-points or more, still even more preferably by 10%-points or more. There is no upper limit for the extend of the increase as long as no lesions and/or damaging of skin and/or capillaries of the skin is caused, however, usually, the sO₂ is increased by 200%-points or less, preferably 60%-points or less, preferably 30%-points or less. More preferably, the sO₂ is increased in the range of 2%-points or more and 200%-points or less, even more preferably 4%-points or more and 60%-points or less, still more preferably 6%-points or more and 60%-points or less, still even more preferably 9%-points or more and 30%-points or less, further preferably 10%-points or more and 30%-points or less; and/or
the rHb is increased:
more preferably, by 5% or more, even more preferably by 9% or more, still more preferably by 13% or more, still even more preferably by 20% or more. There is no upper limit for the extend of the increase as long as no lesions and/or damaging of skin and/or capillaries of the skin is caused, however, usually, the rHb is increased by 200% or less, preferably 100% or less, more preferably 70% or less. More preferably, the rHb is increased in the range of 2% or more and 200% or less, even more preferably 5% or more and 100% or less, still more preferably 9% or more and 100% or less, still even more preferably 13% or more and 70% or less, further preferably 20% or more and 70% or less; and/or
more preferably, by 5 AU or more, even more preferably by 7 AU or more, still more preferably by 9 AU or more, still even more preferably by 12 AU or more. There is no upper limit for the extend in the increase as long as no lesions and/or damaging of skin and/or capillaries of the skin is caused, however, usually, the rHb is increased by 200 AU or less, preferably 80 AU or less, more preferably 30 AU or less. More preferably, the rHb is increased in the range of 2 AU or more and 200 AU or less, even more preferably 5 AU or more and 80 AU or less, still more preferably 7 AU or more and 30 AU or less, still even more preferably 9 AU or more and 30 AU or less, further preferably 12 AU or more and 30 AU or less; and/or
the rFlow is increased:
more preferably, by 20% or more, even more preferably by 70% or more, still more preferably by 140% or more, still even more preferably by 200% or more. There is no upper limit for the extend of the increase as long as no lesions and/or damaging of skin and/or capillaries of the skin is caused, however, usually, the rFlow is increased by 1000% or less, preferably 600% or less, more preferably 500% or less. More preferably, the rFlow is increased in the range of 10% or more and 1000% or less, even more preferably 20% or more and 1000% or less, still more preferably 70% or more and 600% or less, still even more preferably 140% or more and 500% or less, further preferably 200% or more and 500% or less; and/or more preferably, by 40 AU or more, even more preferably by 90 AU or more. There is no upper limit for the extend in the increase as long as no lesions and/or damaging of skin and/or capillaries of the skin is caused, however, usually, the Vel is increased by 500 AU or less, preferably 200 AU or less, more preferably 150 AU or less. More preferably, the Vel is increased in the range of 20 AU or more and 500 AU or less, even more preferably 40 AU or more and 200 AU or less, still more preferably 90 AU or more and 150 AU or less; and/or
the Vel is increased:
more preferably, by 20% or more, even more preferably by 50% or more, still more preferably by 80% or more. There is no upper limit for the extend of the increase as long as no lesions and/or damaging of skin and/or capillaries of the skin is caused, however, usually, the Vel is increased by 500% or less, preferably 300% or less, more preferably 200% or less. More preferably, the Vel is increased in the range of 10% or more and 500% or less, even more preferably 20% or more and 500% or less, still more preferably 80% or more and 300% or less, still even more preferably 80% or more and 200% or less; and/or
more preferably, by 5 AU or more, even more preferably by 7 AU or more, still more preferably by 10 AU or more. There is no upper limit for the extend in the increase as long as no lesions and/or damaging of skin and/or capillaries of the skin is caused, however, usually, the Vel is increased by 150 AU or less, preferably 30 AU or less, more preferably 20 AU or less. More preferably, the Vel is increased in the range of 3 AU or more and 150 AU or less, even more preferably 5 AU or more and 60 AU or less, still more preferably 7 AU or more and 30 AU or less, still even more preferably 10 AU or more and AU or less.

Preferably, in any of the embodiments described herein and particularly in step (A-3), the increased sO₂, the increased rHb, the increased rFlow and/or the increased Vel, whether specified in %, ° C. and/or AU, is indicated with reference to the sO₂, rHb, rFlow and/or Vel, respectively, of untreated skin. More preferably, the skin in step (A-3) is of a skin area, preferably of a skin area [a], and the untreated skin is of an untreated skin area, wherein the untreated skin area is distanced apart from the skin area by 1 cm or more and 10 cm or less. The distance is preferably measured from a point on the outline of the skin area to a point on the outline of the untreated skin area, wherein the points with the smallest distance to each other are taken. More preferably, the untreated skin is the skin before the method of the present invention is carried out on the skin. Preferably, it is the skin of an arm or leg, more preferably of a human.

Typically, untreated skin has a sO₂ of 85% or less, more typically in the range of 30% or more and 85% or less, even more typically, 55% or more and 80% or less, preferably when the sO₂ is measured as described in the Materials and Methods' section under the topic ‘O2C—Oxygen to See’.

Typically untreated skin has a rHb of 90 AU or less, more typically in the range of 30 AU or more and 90 AU or less, even more typically of 60 AU or more and 85 AU or less, preferably when the rHb is measured as described in the Materials and Methods' section under the topic ‘O2C—Oxygen to See’.

Typically, untreated skin has a rFlow of 110 AU or less, more typically in the range of 5 AU or more and 110 AU or less, even more typically of 10 AU or more and 80 AU or less, preferably when the rFlow is measured as described in the Materials and Methods' section under the topic ‘O2C—Oxygen to See’.

Typically, untreated skin has a Vel of 20 AU or less, more typically in the range of 5 AU or more and 20 AU or less, even more typically of 10 AU or more and 18 AU or less, preferably when the Vel is measured as described in the Materials and Methods' section under the topic ‘O2C—Oxygen to See’.

Preferably, in any of the embodiments described herein and particularly in step (A-3), the increased sO₂, the increased rHb, increased rFlow and/or increased Vel is established within 3 hours or less, even more preferably 1.5 hours or less, still more preferably 1 hour or less, still even more preferably 0.75 hours or less, further preferably 0.5 hours or less. Preferably the increased sO₂, the increased rHb, increased rFlow and/or increased Vel is established immediately up to 3 hours, more preferably 0.05 sec up to 1.5 hours, even more preferably 0.5 sec up to 1.5 hours, still more preferably 5 sec up to 1 hour, still even more preferably 10 sec up to 0.75 hours, further preferably 10 sec up to 0.5 hours, even further preferably 10 sec up to 0.25 hours.

Preferably, in any of the embodiments described herein and particularly in step (A-3), the increased sO₂, the increased rHb, increased rFlow and/or increased Vel is present, sustains and/or is maintained for a duration of time of 2 min or more, more preferably 10 min or more, even more preferably for 0.25 hours or more. There is no upper limit for the persistence of the increased sO₂, the increased rHb, increased rFlow and/or increased Vel, however, usually it is for a duration of time of 6 hours or less, more preferably 3 hours or less, even more preferably 1.5 hour or less, still more preferably 1 hour or less, still even more preferably 0.75 hours or less. Preferably the increased sO₂, the increased rHb, increased rFlow and/or increased Vel is present, sustains and/or is maintained for a duration of time in the range of 2 min or more and 6 hours or less, more preferably 5 min or more and 3 hours or less, even more preferably 10 min or more and 1.5 hour or less, still more preferably 0.25 hours or more and 1 hour or less, still even more preferably 0.25 hours or more and 0.75 hours or less.

The duration of time may be continuous or with interruptions, i.e. distributed to two or more sub-durations. Therefore, the duration of time is preferably the total duration of time which may be composed of a single continuous duration or the sum of two or more sub-durations.

Furthermore, the duration or total duration of time for which the time the increased sO₂, the increased rHb, increased rFlow and/or increased Vel is present and/or is maintained after performing any or all of steps (B), (B₁) and/or (C). Such the duration of time is for instance from at the same time when performing or immediately after performing any or all of steps (B), (B₁) and/or (C) to 15 hours or less, preferably 10 hours or less, more preferably 8 hours or less, even more preferably 6 hours or less, even more preferably 2 hours or less. Hence, the time the increased sO₂, the increased rHb, increased rFlow and/or increased Vel may for instance be present and/or maintained for the total duration of time by performing step (A-3) multiple times, e.g. by applying a heat pad several times to the skin or using a continuously warming heat patch. However, it is noted that this does not exclude that step (A-3) is performed before any or all of steps (B), (B₁) and/or (C) are performed.

Preferably, in any of the embodiments described herein and particularly in step (A-3), the generating of the increased sO₂, the increased rHb, increased rFlow and/or increased Vel of the capillaries may be, preferably is accomplished by any means, method(s), substance(s) and/or procedure(s) as long as it is suitable and/or sufficient to generate an increased sO₂, the increased rHb, increased rFlow and/or increased Vel within the skin and/or without damaging the skin like burning, causing lesions, rupture of capillaries and/or reflexive closure of the capillaries.

Preferably, in any of the embodiments described herein and particularly in step (A-3), the generating of the increased sO₂, the increased rHb, increased rFlow and/or increased Vel within the skin is generated by:
(A-0) generating an accumulation of PBMCs within the skin, wherein it is to be understood that preferably any embodiment or definition thereof which is described under the outline note ‘As further regards step (A-0) for any of the embodiments as described herein’ is independently and mutatis mutandis applicable; and/or
(A-1) generating a vasodilation of the capillaries within the skin, wherein it is to be understood that preferably any embodiment or definition thereof which is described under the outline note ‘As further regards step (A-1) for any of the embodiments as described herein’ is independently and mutatis mutandis applicable; and/or
(A-2) generating an increased blood volume within the skin, wherein it is to be understood that preferably any embodiment or definition thereof which is described under the outline note ‘As further regards step (A-2) for any of the embodiments as described herein’ is independently and mutatis mutandis applicable; and/or
(A-4) generating an increased temperature on the skin, wherein it is to be understood that preferably any embodiment or definition thereof which is described under the outline note ‘As further regards step (A-4) for any of the embodiments as described herein’ is independently and mutatis mutandis applicable; and/or
(A-5) generating a redness on the skin, wherein it is to be understood that preferably any embodiment or definition thereof which is described under the outline note ‘As further regards step (A-5) for any of the embodiments as described herein’ is independently and mutatis mutandis applicable; and/or
(A-6) administering conditioning energy to the skin, wherein it is to be understood that preferably any embodiment or definition thereof which is described under the outline note ‘As further regards step (A-6) for any of the embodiments as described herein’ is independently and mutatis mutandis applicable; and/or
(A-7) administering a skin-conditioning agent to the skin, wherein it is to be understood that preferably any embodiment or definition thereof which is described under the outline note ‘As further regards step (A-7) for any of the embodiments as described herein’ is independently and mutatis mutandis applicable.

Preferably, steps (A-0) to (A-2) and (A-4) to (A-7) may not be mutually exclusive.

Preferably, in step (A-3) in any of the embodiments described herein, the skin is of a skin area.

Preferably, in step (A-3) in any of the embodiments described herein, increased sO₂, the increased rHb, increased rFlow and/or increased Vel is generated within the skin of the skin area.

More preferably, the increased sO₂, the increased rHb, increased rFlow and/or increased Vel is generated within the skin of the skin area and the conditioning energy and/or skin-conditioning agent is administered to the skin of the skin area.

Even more preferably, the increased sO₂, the increased rHb, increased rFlow and/or increased Vel is generated within the skin of the skin area, wherein the conditioning energy and/or skin-conditioning agent is administered to the skin of the skin area by applying the conditioning energy and/or skin-conditioning agent to the skin of the application area.

Preferably, in step (A-3) in any of the embodiments described herein, the application area is that of the respective skin area.

More preferably, the skin area is the skin area [a] and/or the application area is the application area [a].

Preferably, in step (A-3) in any of the embodiments described herein, the skin area and the skin area [a] are the same skin area and skin area [a], respectively, as mentioned in any of the embodiments described herein. If not mentioned otherwise, it is to be understood that any embodiment or definition described herein in terms of step (A) and a skin area or skin area [a] is independently and mutatis mutandis applicable to the skin area and the skin area [a], respectively, of step (A-3).

Preferably, in step (A-3) in any of the embodiments described herein, the application area and the application area [a] are the same application area and application area [a], respectively, as mentioned in any of the embodiments described herein. If not mentioned otherwise, it is to be understood that any embodiment or definition described herein in terms of step (A) and an application area or application area [a] is independently and mutatis mutandis applicable to the application area and the application area [a], respectively, of step (A-3).

It is to be understood that any of the embodiments mentioned under the outline note ‘As further regards step (A-3) for any of the embodiments as described herein’ is independently applicable and/or combinable with any of the embodiments for the method as described herein and particularly with any embodiment of step (A) of the method as described herein.

As further regards step (A-4) for any of the embodiments as described herein

As stated above, step (A-4) requires generating an increased temperature on the skin of the subject.

Preferably, in any of the embodiments described herein and particularly in step (A-4), the temperature is increased to any extent and/or duration suitable to achieve the beneficial effects, is pharmaceutically acceptable and/or does not cause lesions and/or a damaging of skin like burning, causing lesions, rupture of capillaries and/or reflexive closure of the capillaries.

Preferably, in any of the embodiments described herein and particularly in step (A-4), the temperature may be increased to any extent and/or any duration as long as it is pharmaceutically acceptable and does not cause lesions and/or a damaging of skin like burning, causing lesions, rupture of capillaries and/or reflexive closure of the capillaries.

Preferably, in any of the embodiments described herein and particularly in step (A-4), the increase of the temperature is palpable with the fingers.

Preferably, in any of the embodiments described herein and particularly in step (A-4),
the increased temperature on the skin is indicated by; and/or
the temperature on the skin is increased to generate, preferably temperature on the skin is increased to an extent and/or a duration is suitable and/or sufficient to generate:
   an accumulation of PBMCs within the skin, wherein it is to be understood that preferably any embodiment or definition thereof which is described under the outline note ‘As further regards step (A-0) for any of the embodiments as described herein’ is independently and mutatis mutandis applicable; and/or
   a vasodilation of the capillaries within the skin, wherein it is to be understood that preferably any embodiment or definition thereof which is described under the outline note ‘As further regards step (A-1) for any of the embodiments as described herein’ is independently and mutatis mutandis applicable; and/or
   an increased blood volume within the skin, wherein it is to be understood that preferably any embodiment or definition thereof which is described under the outline note ‘As further regards. step (A-2) for any of the embodiments as described herein’ is independently and mutatis mutandis applicable; and/or
   an increased sO₂ and/or an increased rHb within the skin, wherein it is to be understood that preferably any embodiment or definition thereof which is described under the outline note ‘As further regards. step (A-3) for any of the embodiments as described herein’ is independently and mutatis mutandis applicable; and/or
   a redness on the skin, wherein it is to be understood that preferably any embodiment or definition thereof which is described under the outline note ‘As further regards. step (A-5) for any of the embodiments as described herein’ is independently and mutatis mutandis applicable.

Preferably, in any of the embodiments described herein and particularly in step (A-4), the temperature on the skin is increased by 1% or more, preferably when referred to ° C. (degrees Celsius), and/or is increased by 0.2° C. or more.

Preferably, in any of the embodiments described herein and particularly in step (A-4), the temperature on the skin may be measured by any suitable method known to the person skilled in the art, preferably it is measured as described in the Materials and Methods' section under the topic ‘Thermal measurement and skin reddening’.

The temperature on the skin is increased:
more preferably, by 3% or more, even more preferably by 5% or more, still more preferably by 8% or more, still even more preferably by 9% or more, further preferably by 10% or more, preferably when referred to ° C. (degrees Celsius), wherein preferably there is no upper limit for the increase as long as no burning lesions and/or damaging of skin like burning, causing lesions, rupture of capillaries and/or reflexive closure of the capillaries is caused, however, usually the increase of the temperature is 90% or less, preferably 70% or less, more preferably 50% or less, still more preferably 20% or less, still even more preferably 15% or less. Preferably, the temperature on the skin is increased in the range of 1% or more and 90% or less, more preferably 3% or more and 70% or less, even more preferably by 5% or more and 70% or less, still more preferably by 8% or more and 50% or less, still even more preferably by 9% or more and 20% or less, further preferably by 10% or more and 15% or less; and/or
more preferably, by 0.3° C. or more, even more preferably by 0.5° C. or more, still more preferably by 0.8° C. or more, still even more preferably by 1° C. or more, further preferably by 1.5° C. or more, even further preferably by 2° C. or more, still further preferably by 3° C. or more, wherein preferably there is no upper limit for the increase as long as no burning lesions and/or damaging of skin like burning, causing lesions, rupture of capillaries and/or reflexive closure of the capillaries is caused, however, usually, the increase of the temperature is 30° C. or less, preferably 25° C. or less, even more preferably 20° C. or less, still more preferably 15° C. or less, still even more preferably 8° C. or less, further preferably 5° C. or less.

Preferably, the temperature on the skin is increased in the range of 0.2° C. or more and 30° C. or less, more preferably 0.3° C. or more and 30° C. or less, even more preferably 0.5° C. or and 25° C. or less, still more preferably by 0.8° C. or more and 25° C. or less, still even more preferably by 1° C. or more and 20° C. or less, further preferably by 1.5° C. or more and 15° C. or less, even further preferably by 2° C. or more and 8° C. or less, still further preferably by 3° C. or more and 5° C. or less. Preferably, in any of the embodiments described herein and particularly in step (A-4), the increase of the temperature on the skin, whether specified in % and/or in ° C., is indicated with reference to the temperature of untreated skin. More preferably, the skin in step (A-4) is of a skin area, preferably of a skin area [a], and the untreated skin is of an untreated skin area, wherein the untreated skin area is distanced apart from the skin area by 1 cm or more and 10 cm or less. The distance is preferably measured from a point on the outline of the skin area to a point on the outline of the untreated skin area, wherein the points with the smallest distance to each other are taken. More preferably, the untreated skin is the skin before the method of the present invention is carried out on the skin. Preferably, the untreated skin is of an arm or leg, more preferably a human.

Typically, untreated skin has a temperature, preferably a skin surface temperature, of 36° C. or less, more typically in the range of 20° C. or more and 36° C. or less, even more typically 22° C. or more and 34° C. or less, still more typically 24° C. or more and 34° C. or less, still even more typically 24° C. or more and 32° C. or less, preferably when measured as described in the Materials and Methods' section ‘Thermal measurement and skin reddening’.

Preferably, in any of the embodiments described herein and particularly in step (A-4), the increased temperature on the skin is established within 3 hours or less, even more preferably 1.5 hours or less, still more preferably 1 hour or less, still even more preferably 0.75 hours or less, further preferably 0.5 hours or less. Preferably the increased temperature on the skin is established immediately up to 3 hours, more preferably 0.05 sec up to 1.5 hours, even more preferably 0.5 sec up to 1.5 hours, still more preferably 5 sec up to 1 hour, still even more preferably 10 sec up to 0.75 hours, further preferably 10 sec up to 0.5 hours, even further preferably 10 sec up to 0.25 hours.

Preferably, in any of the embodiments described herein and particularly in step (A-4), the increased temperature on the skin is present, sustains and/or is maintained for a duration of time of 2 min or more, more preferably 10 min or more, even more preferably for 0.25 hours or more. There is no upper limit for the persistence of the increased temperature on the skin, however, usually it is for a duration of time of 6 hours or less, more preferably 3 hours or less, even more preferably 1.5 hour or less, still more preferably 1 hour or less, still even more preferably 0.75 hours or less. Preferably the increased temperature on the skin is present, sustains and/or is maintained for a duration of time in the range of 2 min or more and 6 hours or less, more preferably 5 min or more and 3 hours or less, even more preferably 10 min or more and 1.5 hour or less, still more preferably 0.25 hours or more and 1 hour or less, still even more preferably 0.25 hours or more and 0.75 hours or less.

The duration of time may be continuous or with interruptions, i.e. distributed to two or more sub-durations. Therefore, the duration of time is preferably the total duration of time which may be composed of a single continuous duration or the sum of two or more sub-durations.

Furthermore, the duration or total duration of time for which the time the increased temperature is present and/or is maintained after performing any or all of steps (B), (B₁) and/or (C). Such the duration of time is for instance from at the same time when performing or immediately after performing any or all of steps (B), (B₁) and/or (C) to 15 hours or less, preferably 10 hours or less, more preferably 8 hours or less, even more preferably 6 hours or less, even more preferably 2 hours or less. Hence, the time the increased temperature may for instance be present and/or maintained for the total duration of time by performing step (A-4) multiple times, e.g. by applying a heat pad several times to the skin or using a continuously warming heat patch. However, it is noted that this does not exclude that step (A-4) is performed before any or all of steps (B), (B₁) and/or (C) are performed.

Preferably, in any of the embodiments described herein and particularly in step (A-4), the generating of the increased temperature on the skin may be, preferably is accomplished by any means, method(s), substance(s) and/or procedure(s) as long as it is suitable and/or sufficient to generate an increased temperature on the skin and without damaging the skin like burning, causing lesions, rupture of capillaries and/or reflexive closure of the capillaries.

Preferably, in any of the embodiments described herein and particularly in step (A-4), the generating of the increased temperature is generated by:
(A-0) generating an accumulation of PBMCs within the skin, wherein it is to be understood that preferably any embodiment or definition thereof which is described under the outline note ‘As further regards step (A-0) for any of the embodiments as described herein’ is independently and mutatis mutandis applicable; and/or
(A-1) generating a vasodilation of the capillaries within the skin, wherein it is to be understood that preferably any embodiment or definition thereof which is described under the outline note ‘As further regards step (A-1) for any of the embodiments as described herein’ is independently and mutatis mutandis applicable; and/or
(A-2) generating an increased blood volume within the skin, wherein it is to be understood that preferably any embodiment or definition thereof which is described under the outline note ‘As further regards step (A-2) for any of the embodiments as described herein’ is independently and mutatis mutandis applicable; and/or
(A-3) generating an increased sO₂ and/or an increased rHb within the skin, wherein it is to be understood that preferably any embodiment or definition thereof which is described under the outline note ‘As further regards step (A-3) for any of the embodiments as described herein’ is independently and mutatis mutandis applicable; and/or
(A-5) generating a redness on the skin, wherein it is to be understood that preferably any embodiment or definition thereof which is described under the outline note ‘As further regards step (A-5) for any of the embodiments as described herein’ is independently and mutatis mutandis applicable; and/or
(A-6) administering conditioning energy to the skin, wherein it is to be understood that preferably any embodiment or definition thereof which is described under the outline note ‘As further regards step (A-6) for any of the embodiments as described herein’ is independently and mutatis mutandis applicable; and/or
(A-7) administering a skin-conditioning agent to the skin, wherein it is to be understood that preferably any embodiment or definition thereof which is described under the outline note ‘As further regards step (A-7) for any of the embodiments as described herein’ is independently and mutatis mutandis applicable.

Preferably, steps (A-0) to (A-3) and (A-5) to (A-7) may not be mutually exclusive.

Preferably, in step (A-4) in any of the embodiments described herein, the skin is of a skin area.

Preferably, in step (A-4) in any of the embodiments described herein, the increased temperature is generated within the skin of the skin area.

More preferably, the increased temperature is generated within the skin of the skin area and the conditioning energy and/or skin-conditioning agent is administered to the skin of the skin area.

Even more preferably, the increased temperature is generated within the skin of the skin area, wherein the conditioning energy and/or skin-conditioning agent is administered to the skin of the skin area by applying the conditioning energy and/or skin-conditioning agent to the skin of the application area.

Preferably, in step (A-4) in any of the embodiments described herein, the application area is that of the respective skin area. More preferably, the skin area is the skin area [a] and/or the application area is the application area [a].

Preferably, in step (A-4) in any of the embodiments described herein, the skin area and the skin area [a] are the same skin area and skin area [a], respectively, as mentioned in any of the embodiments described herein. If not mentioned otherwise, it is to be understood that any embodiment or definition described herein in terms of step (A) and a skin area or skin area [a] is independently and mutatis mutandis applicable to the skin area and the skin area [a], respectively, of step (A-4).

Preferably, in step (A-4), the application area and the application area [a] are the same application area and application area [a], respectively, as mentioned in any of the embodiments described herein. If not mentioned otherwise, it is to be understood that any embodiment or definition described herein in terms of step (A) and an application area or application area [a] is independently and mutatis mutandis applicable to the application area and the application area [a], respectively, of step (A-4).

In any of the embodiments described herein, the expressions “temperature on the skin”, “temperature of the skin” and/or “skin surface temperature” may be used interchangeably.

It is to be understood that any of the embodiments mentioned under the outline note ‘As further regards step (A-4) for any of the embodiments as described herein’ is independently applicable and/or combinable with any of the embodiments for the method as described herein and particularly with any embodiment of step (A) of the method as described herein.

As further regards step (A-5) for any of the embodiments as described herein

As stated above, step (A-5) requires generating a redness on the skin of the subject.

Preferably, in any of the embodiments described herein and particularly in step (A-5), the redness is on the surface of the skin.

Preferably, i in any of the embodiments described herein and particularly n step (A-5), the redness is increased to any extent and/or duration suitable to achieve the beneficial effects, is pharmaceutically acceptable and/or does not cause lesions and/or a damaging of skin like burning, causing lesions, rupture of capillaries and/or reflexive closure of the capillaries.

Preferably, in any of the embodiments described herein and particularly in step (A-5), the redness may be increased to any extent and/or any duration as long as it is pharmaceutically acceptable and does not cause lesions and/or a damaging of skin like burning, causing lesions, rupture of capillaries and/or reflexive closure of the capillaries.

Preferably, in any of the embodiments described herein and particularly in step (A-5), the redness is visually detectable to a naked human eye.

Preferably, in any of the embodiments described herein and particularly in step (A-5), the redness is visually detectable to a naked human eye under day light conditions and/or artificial light conditions.

Preferably, in any of the embodiments described herein and particularly in step (A-5),
the redness on the skin is indicated by; and/or
the redness on the skin is to generate, preferably the redness to an extent and/or a duration is suitable and/or sufficient to generate:
   an accumulation of PBMCs within the skin, wherein it is to be understood that preferably any embodiment or definition thereof which is described under the outline note ‘As further regards step (A-0) for any of the embodiments as described herein’ is independently and mutatis mutandis applicable; and/or
   a vasodilation of the capillaries within the skin, wherein it is to be understood that preferably any embodiment or definition thereof which is described under the outline note ‘As further regards step (A-1) for any of the embodiments as described herein’ is independently and mutatis mutandis applicable; and/or
   an increased blood volume within the skin, wherein it is to be understood that preferably any embodiment or definition thereof which is described under the outline note ‘As further regards step (A-2) for any of the embodiments as described herein’ is independently and mutatis mutandis applicable; and/or
   an increased sO₂ and/or an increased rHb within the skin, wherein it is to be understood that preferably any embodiment or definition thereof which is described under the outline note ‘As further regards step (A-3) for any of the embodiments as described herein’ is independently and mutatis mutandis applicable; and/or an increased temperature on the skin, wherein it is to be understood that preferably any embodiment or definition thereof which is described under the outline note ‘As further regards step (A-4) for any of the embodiments as described herein’ is independently and mutatis mutandis applicable.

Preferably, in any of the embodiments described herein and particularly in step (A-5), the redness on the skin may be measured by any suitable method known to the person skilled in the art, preferably the temperature on the skin is measured as described in the Materials and Methods' section ‘Thermal measurement and skin reddening’.

Preferably, in any of the embodiments described herein and particularly in step (A-5), the increase of the redness on the skin is indicated with reference to the temperature of untreated skin. More preferably, the skin in step (A-5) is of a skin area, preferably of a skin area [a], and the untreated skin is of an untreated skin area, wherein the untreated skin area is distanced apart from the skin area by 1 cm or more and 10 cm or less. The distance is preferably measured from a point on the outline of the skin area to a point on the outline of the untreated skin area, wherein the points with the smallest distance to each other are taken. Preferably, the untreated skin is of an arm or leg, more preferably a human.

Preferably, in any of the embodiments described herein and particularly in step (A-5), the redness on the skin is present, sustains and/or is maintained for a duration of time of 2 min or more, more preferably 10 min or more, even more preferably for 0.25 hours or more. There is no upper limit for the persistence of the vasodilation, increased blood volume, increased sO₂ and/or increased rHb, increased temperature and/or redness, however, usually it is for a duration of time of 6 hours or less, more preferably 3 hours or less, even more preferably 1.5 hour or less, still more preferably 1 hour or less, still even more preferably 0.75 hours or less. Preferably the vasodilation, increased blood volume, increased sO₂ and/or increased rHb, increased temperature and/or redness is present, sustains and/or is maintained for a duration of time in the range of 2 min or more and 6 hours or less, more preferably 5 min or more and 3 hours or less, even more preferably 10 min or more and 1.5 hour or less, still more preferably 0.25 hours or more and 1 hour or less, still even more preferably 0.25 hours or more and 0.75 hours or less.

The duration of time may be continuous or with interruptions, i.e. distributed to two or more sub-durations. Therefore, the duration of time is preferably the total duration of time which may be composed of a single continuous duration or the sum of two or more sub-durations.

Furthermore, the duration or total duration of time for which the time the redness is present and/or is maintained after performing any or all of steps (B), (B₁) and/or (C). Such the duration of time is for instance from at the same time when performing or immediately after performing any or all of steps (B), (B₁) and/or (C) to 15 hours or less, preferably 10 hours or less, more preferably 8 hours or less, even more preferably 6 hours or less, even more preferably 2 hours or less. Hence, the time the redness may for instance be present and/or maintained for the total duration of time by performing step (A-5) multiple times, e.g. by applying a heat pad several times to the skin or using a continuously warming heat patch. However, it is noted that this does not exclude that step (A-5) is performed before any or all of steps (B), (B₁) and/or (C) are performed.

Preferably, in any of the embodiments described herein and particularly in step (A-5), the redness on the skin is indicated with reference to untreated skin. More preferably, the skin in step (A-5) is of a skin area, preferably of a skin area [a], and the untreated skin is of an untreated skin area, wherein the untreated skin area is distanced apart from the skin area by 1 cm or more and 10 cm or less. The distance is preferably measured from a point on the outline of the skin area to a point on the outline of the untreated skin area, wherein the points with the smallest distance to each other are taken.

Preferably, in any of the embodiments described herein and particularly in step (A-5), the generating of the redness on the skin may be, preferably is accomplished by any means, method(s), substance(s) and/or procedure(s) as long as it is suitable and/or sufficient to generate a redness on the skin and/or without damaging the skin like burning, causing lesions, rupture of capillaries and/or reflexive closure of the capillaries.

Preferably, in any of the embodiments described herein and particularly in step (A-5), the generating of the redness is generated by:
(A-0) generating an accumulation of PBMCs within the skin, wherein it is to be understood that preferably any embodiment or definition thereof which is described under the outline note ‘As further regards step (A-0) for any of the embodiments as described herein’ is independently and mutatis mutandis applicable; and/or
(A-1) generating a vasodilation of the capillaries within the skin, wherein it is to be understood that preferably any embodiment or definition thereof which is described under the outline note ‘As further regards step (A-1) for any of the embodiments as described herein’ is independently and mutatis mutandis applicable; and/or
(A-2) generating an increased blood volume within the skin, wherein it is to be understood that preferably any embodiment or definition thereof which is described under the outline note ‘As further regards step (A-2) for any of the embodiments as described herein’ is independently and mutatis mutandis applicable; and/or
(A-3) generating an increased sO₂ and/or an increased rHb within the skin, wherein it is to be understood that preferably any embodiment or definition thereof which is described under the outline note ‘As further regards step (A-3) for any of the embodiments as described herein’ is independently and mutatis mutandis applicable; and/or
(A-4) generating an increased temperature on the skin, wherein mutatis mutandis as mentioned in any of the embodiments of the present invention described under the outline note ‘As further regards step (A-4) for any of the embodiments as described herein’; and/or
(A-6) administering conditioning energy to the skin, wherein it is to be understood that preferably any embodiment or definition thereof which is described under the outline note ‘As further regards step (A-6) for any of the embodiments as described herein’ is independently and mutatis mutandis applicable; and/or
(A-7) administering a skin-conditioning agent to the skin, wherein it is to be understood that preferably any embodiment or definition thereof which is described under the outline note ‘As further regards step (A-7) for any of the embodiments as described herein’ is independently and mutatis mutandis applicable.

Preferably, steps (A-0) to (A-4), (A-6) and (A-7) may not be mutually exclusive.

Preferably, in step (A-5) in any of the embodiments described herein, the skin is of a skin area.

Preferably, in step (A-5) in any of the embodiments described herein, the redness is generated within the skin of the skin area.

More preferably, the redness is generated within the skin of the skin area and the conditioning energy and/or skin-conditioning agent is administered to the skin of the skin area.

Even more preferably, the redness is generated within the skin of the skin area, wherein the conditioning energy and/or skin-conditioning agent is administered to the skin of the skin area by applying the conditioning energy and/or skin-conditioning agent to the skin of the application area.

Preferably, in step (A-5) in any of the embodiments described herein, the application area is that of the respective skin area. More preferably, the skin area is the skin area [a] and/or the application area is the application area [a].

Preferably, in step (A-5) in any of the embodiments described herein, the skin area and the skin area [a] are the same skin area and skin area [a], respectively, as mentioned in any of the embodiments described herein. If not mentioned otherwise, it is to be understood that any embodiment or definition described herein in terms of step (A) and a skin area or skin area [a] is independently and mutatis mutandis applicable to the skin area and the skin area [a], respectively, of step (A-5).

Preferably, in step (A-5) in any of the embodiments described herein, the application area and the application area [a] are the same application area and application area [a], respectively, as mentioned in any of the embodiments described herein. If not mentioned otherwise, it is to be understood that any embodiment or definition described herein in terms of step (A) and an application area or application area [a] is independently and mutatis mutandis applicable to the application area and the application area [a], respectively, of step (A-5).

In any of the embodiments described herein, the expressions “redness on the skin”, “redness of the skin”, “skin redness” and/or “skin surface redness” may be used interchangeably.

It is to be understood that any of the embodiments mentioned under the outline note ‘As further regards step (A-5) for any of the embodiments as described herein’ is independently applicable and/or combinable with any of the embodiments for the method as described herein and particularly with any embodiment of step (A) of the method as described herein.

As further regards step (A-6) for any of the embodiments as described herein

As stated above, step (A-6) requires administering conditioning energy to the skin of the subject.

Preferably, step (A-6) requires applying conditioning energy to the skin of the subject.

Preferably, in any of the embodiments described herein and particularly in step (A-6), the conditioning energy is administered and/or applied to the surface of the skin.

In any of the embodiments described herein and particularly in step (A-6), the conditioning energy may be any energy known to the person skilled in the art, which is pharmaceutically acceptable and/or does not cause lesions and/or a damaging of skin like burning, causing lesions, rupture of capillaries and/or reflexive closure of the capillaries. For instance, the conditioning energy is in a form of work; heat; electromagnetic radiation; microwaves; thermal radiation; infrared radiation; ultraviolet light; visible light; mechanical agitation; mechanical work; electrical work; electric current; ultrasonic; mechanical agitation; a massage like a manual or machined skin massage using e.g. vibration and/or rubbing of the skin; vibration; rubbing; friction; negative pressure e.g. using sucking like machined, manual or oral sucking; manually or machined generated negative pressure using e.g. cupping particularly dry cupping or cupping without injuring the skin; hot air; hyperthermia and/or warm flowing fluid like warm water, or any combination thereof.

Preferably, the conditioning energy is any energy for modifying a condition of the skin particularly in respect of the amount of PBMCs, the vasodilation of the capillaries within the skin, the blood volume within the skin, the sO₂ within the skin, the rHb within the skin, the temperature on the skin and/or the redness on the skin.

In any of the embodiments described herein and particularly in step (A-6), the conditioning energy may be administered and/or applied to the skin to any extent and/or any duration as long as it is pharmaceutically acceptable and does not cause lesions and/or a damaging of skin like burning, causing lesions, rupture of capillaries and/or reflexive closure of the capillaries.

Preferably, in any of the embodiments described herein and particularly in step (A-6), the conditioning energy is administered and/or applied by using an energizing means. The energizing means may be any energizing means known to the person skilled in the art, which is pharmaceutically acceptable and/or does not cause lesions and/or a damaging of skin like burning, causing lesions, rupture of capillaries and/or reflexive closure of the capillaries.

Preferably, in any of the embodiments described herein and particularly in step (A-6), the energizing means is any means suitable and/or configured for delivering, administering and/or applying the conditioning energy to the skin, preferably to the surface of the skin.

Preferably, in any of the embodiments described herein and particularly in step (A-6), the energizing means is suitable and/or configured for delivering, administering and/or applying the conditioning energy to any extent and/or any duration as long as it is pharmaceutically acceptable and does not cause lesions and/or a damaging of skin like burning, causing lesions, rupture of capillaries and/or reflexive closure of the capillaries.

In any of the embodiments described herein and particularly in step (A-6), the conditioning energy may also be referred to as energy or skin-conditioning energy.

Preferably, in any of the embodiments described herein and particularly in step (A-6), the energizing means is suitable and/or configured for delivering, administering and/or applying, and/or delivers, administers and/or applies the conditioning energy to the skin by work, electromagnetic waves, radiation, electric current, heat e.g. by physical processes like conduction due to mechanical contact to the skin, by convection due to entrainment of thermal energy in a flowing fluid, mechanical coupling, inductive coupling, thermal radiation using electromagnetic waves, visible light, mechanical agitation, negative pressure and/or by inducing biochemical processes like by administering and/or applying the skin-conditioning agent as mentioned in any of the embodiments described herein to the skin, or any combination thereof.

More preferably, in any of the embodiments described herein and particularly in step (A-6), the energizing means may be an apparative energizing means or a non-apparative energizing means, preferably an apparative energizing means.

Hence, the energizing means and/or apparative energizing means may for instance be any means emitting thermal radiation; means emitting infrared radiation; means emitting ultrasonic; means emitting electromagnetic waves; means emitting microwaves; means emitting ultraviolet light; means emitting radiation like visible light; means emitting hot air or warm fluid; means providing electric current to the skin; means providing heat to the skin; a warm or warming object like a heater, a heat pad e.g. equipped with a latent heat storage, an electrical warmer e.g. an electrical hand warmer, a preheated object or a warm tea cup; means for moxibustion; means for performing work on the skin; means for massage like machines suitable for skin massage like a facial massager and/or vibrating machine; means for generating vibration and/or rubbing; means for providing mechanical agitation to the skin of the subject; means for providing any kind of vacuum or negative pressure to the skin like an apparatus sucking the skin, cupping glasses, cupping machine or vacuum pump; means for providing ultrasonic to the skin; a heat plaster, heat patch, heat pad, heat wadding, heat padding, heat dressing, heat compress, microneedle heat patch, transdermal heat patch, time-release heat plaster, moxa plaster and/or heat bandage; a energizing topical dosage form as mentioned in any of the embodiments described herein, preferably a heating topical dosage form as mentioned in any of the embodiments of the present invention described; and/or a medical device according to any of the embodiments of to the present invention as described herein, etc. Furthermore, the energizing means and/or the non-apparative energizing means may be for instance warm flowing fluid like water e.g. when run over the skin; manual applications like moving hands of a masseur performing e.g. a manual skin massage using vibration, rubbing or generating a sucking action on the skin.

More preferably, in any of the embodiments described herein and particularly in step (A-6), the conditioning energy delivered, administered and/or applied is heat.

Hence, more preferably, step (A-6) requires administering heat to the skin of the subject.

Hence, even more preferably, step (A-6) requires applying heat to the skin of the subject.

Preferably, in any of the embodiments described herein and particularly in step (A-6), the heat administered and/or applied is pharmaceutically acceptable and/or does not cause lesions and/or a damaging of skin like burning, causing lesions, rupture of capillaries and/or reflexive closure of the capillaries.

For instance, the heat administered and/or applied may be in the form of electromagnetic radiation; thermal radiation; microwaves; infrared radiation; hot air; hyperthermia; warm flowing fluid like warm water.

Preferably, in any of the embodiments described herein and particularly in step (A-6), the energizing means is a heating means.

Preferably, in any of the embodiments described herein and particularly in step (A-6), the heat is administered and/or applied by any heating means known to the person skilled in the art, which is pharmaceutically acceptable and/or does not cause lesions and/or a damaging of skin like burning, causing lesions, rupture of capillaries and/or reflexive closure of the capillaries.

Preferably, in any of the embodiments described herein and particularly in step (A-6), the heating means is any means suitable and/or configured for delivering, administering and/or applying heat to the skin, preferably to the surface of the skin.

Preferably, in any of the embodiments described herein and particularly in step (A-6), the heating means is suitable and/or configured for administering and/or applying the heat to any extent and/or any duration as long as it is pharmaceutically acceptable and does not cause lesions and/or a damaging of skin like burning, causing lesions, rupture of capillaries and/or reflexive closure of the capillaries.

Preferably, in any of the embodiments described herein and particularly in step (A-6), the heating means is suitable and/or configured for delivering, administering and/or applying and/or it delivers, administers and/or applies the heat to the skin by physical processes like conduction due to mechanical contact to the skin, by convection due to entrainment of thermal heat in a flowing fluid and/or by thermal radiation using electromagnetic waves.

More preferably, in any of the embodiments described herein and particularly in step (A-6), the heating means may be an apparative heating means or a non-apparative heating means, preferably an apparative heating means.

Hence, the heating means and/or the apparative heating means may for instance be any means emitting thermal radiation; means emitting infrared radiation; means emitting ultrasonic; means emitting electromagnetic waves; means emitting microwaves; means emitting ultraviolet light; means emitting radiation like visible light; means emitting hot air or warm fluid; means providing electric current to the skin; means providing heat to the skin; a warm or warming object like a heater, a heat pad e.g. equipped with a latent heat storage, an electrical warmer e.g. an electrical hand warmer, a preheated object or a warm tea cup; means for moxibustion; means for providing ultrasonic to the skin; a heat plaster, heat patch, heat pad, heat wadding, heat padding, heat dressing, heat compress, microneedle heat patch, transdermal heat patch, time-release heat plaster, moxa plaster and/or heat bandage; a heating topical dosage form, as mentioned in any of the embodiments described herein like; and/or a medical device according to any of the embodiments of the present invention as described herein, etc.

Furthermore, the heating means and/or the non-apparative heating means may for instance be warm flowing fluid like water e.g. when run over the skin and/or manual applications like warm hands of a masseur.

Preferably, in any of the embodiments described herein and particularly in step (A-6),
the conditioning energy and/or the heat is suitable and/or sufficient to generate and/or generates; and/or
the conditioning energy and/or the heat is administered and/or applied to generate, preferably the conditioning energy and/or the heat is administered and/or applied to an extent and/or a duration is suitable and/or sufficient to generate; and/or
the energizing means and/or heating means is suitable, preferably and/or configured, to generate and/or generates:
   an accumulation of PBMCs within the skin, wherein it is to be understood that preferably any embodiment or definition thereof which is described under the outline note ‘As further regards step (A-0) for any of the embodiments as described herein’ is independently and mutatis mutandis applicable; and/or
   a vasodilation of the capillaries within the skin, wherein it is to be understood that preferably any embodiment or definition thereof which is described under the outline note ‘As further regards step (A-1) for any of the embodiments as described herein’ is independently and mutatis mutandis applicable; and/or
   an increased blood volume within the skin, wherein it is to be understood that preferably any embodiment or definition thereof which is described under the outline note ‘As further regards step (A-2) for any of the embodiments as described herein’ is independently and mutatis mutandis applicable; and/or
   an increased sO₂ and/or increased rHb within the skin, wherein it is to be understood that preferably any embodiment or definition thereof which is described under the outline note ‘As further regards step (A-3) for any of the embodiments as described herein’ is independently and mutatis mutandis applicable; and/or
   an increased temperature on the skin, wherein it is to be understood that preferably any embodiment or definition thereof which is described under the outline note ‘As further regards step (A-4) for any of the embodiments as described herein’ is independently and mutatis mutandis applicable; and/or
   a redness on the skin, wherein it is to be understood that preferably any embodiment or definition thereof which is described under the outline note ‘As further regards step (A-5) for any of the embodiments as described herein’ is independently and mutatis mutandis applicable,
more preferably,
   the vasodilation of the capillaries within the skin,
   the increased blood volume within the skin,
   the increased sO₂ and/or the increased rHb within the skin, and/or
   the increased temperature on the skin,
even more preferably,
   the increased blood volume within the skin,
   the increased sO₂ and/or the increased rHb within the skin, and/or
   the increased temperature on the skin.

Preferably, in any of the embodiments described herein and particularly in step (A-6),
the conditioning energy and/or heat is suitable and/or sufficient to generate and/or generates; and/or
the conditioning energy and/or heat is administered and/or applied to generate, preferably the conditioning energy and/or heat is administered and/or applied to an extent and/or a duration is suitable and/or sufficient to generate; and/or
the energizing means and/or heating means is suitable, preferably and/or configured, to generate and/or generates, a temperature on the skin of 25° C. or more, more preferably 27° C. or more, more preferably 28° C. or more, even more preferably 29° C. or more, still more preferably 30° C. or more, still even more preferably 31° C. or more, further preferably 32° C. or more, preferably when measured as described in the Materials and Methods' section ‘Thermal measurement and skin reddening’. There is no upper limit for temperature on the skin generated as long as no burning lesions and/or damaging of skin like burning, causing lesions, rupture of capillaries and/or reflexive closure of the capillaries is caused. However, usually, the temperature on the skin generated is 65° C. or less, preferably 58° C. or less, more preferably 50° C. or less, still more preferably 45° C. or less, still even more preferably 42° C. or less, further preferably 41° C. or less. More preferably, the temperature on the skin generated is in the range of 25° C. or more and 65° C. or less, more preferably 27° C. or more and 58° C. or less, even more preferably 28° C. or more and 50° C. or less, still more preferably 29° C. or more and 45° C. or less, still even more preferably 30° C. or more and 45° C. or less, further preferably 31° C. or more and 42° C. or less, even further preferably 32° C. or more and 41° C. or less.

Preferably, in any of the embodiments described herein and particularly in step (A-6),
the amount of the conditioning energy and/or heat administered and/or applied is; and/or
the energizing means and/or heating means is suitable, preferably and/or configured, to deliver, administer and/or apply an amount of conditioning energy and/or heat of, 0.1 kJ/cm² skin or more (‘kJ/cm² skin’ is in its meaning equivalent to ‘kilojoule/one square centimetre of the skin), more preferably 0.3 kJ/cm² skin or more, even more preferably 0.6 LT/cm² skin or more, still more preferably 1 kJ/cm² skin or more, still even more preferably 2 kJ/cm² skin or more, further preferably 3 kJ/cm² skin or more, even further preferably 4 kJ/cm² skin or more, still further preferably by 6 kJ/cm² skin or more. There is no upper limit for the amount of conditioning energy as long as no burning lesions and/or damaging of skin is caused. However, usually, the amount of conditioning energy administered and/or applied is 60 kJ/cm² skin or less, preferably 50 kJ/cm² skin or less, even more preferably 40 kJ/cm² skin or less, still more preferably 30 kJ/cm² skin or less, still even more preferably 15 kJ/cm² skin or less, further preferably 10 kJ/cm² skin or less. Preferably, the amount of conditioning energy administered and/or applied is in the range of 0.1 kJ/cm² skin or more and 60 kJ/cm² skin or less, more preferably 0.3 LT/cm² skin or more and 60 kJ/cm² skin or less, even more preferably by 0.6 kJ/cm² skin or more and 50 kJ/cm² skin or less, still more preferably by 1 kJ/cm² skin or more and 50 kJ/cm² skin or less, still even more preferably by 2 kJ/cm² skin or more and 40 kJ/cm² skin or less, further preferably by 3 kJ/cm² skin or more and 30 kJ/cm² skin or less, even further preferably by 4 kJ/cm² skin or more and 15 kJ/cm² skin or less, still further preferably by 6 kJ/cm² skin or more and 10 kJ/cm² skin or less.

Preferably, in any of the embodiments described herein and particularly in step (A-6),
the amount of conditioning energy and/or heat is administered and/or applied; and/or
the energizing means and/or heating means is suitable, preferably and/or configured, to deliver, administer and/or apply the amount of conditioning energy,
within 60 min or less, more preferably 30 min or less, even more preferably 15 min or less, still more preferably 5 min or less, still even more preferably 2 min or less. Usually, it is within 1 sec or more, more preferably 5 sec or more, even more preferably 10 sec or more, still more preferably 30 sec or more, still even more preferably 1 min or more. More preferably it is in the range of 1 sec or more and 60 min or less, more preferably 5 sec or more and 30 min or less, even more preferably 10 sec or more and 15 min or less, still more preferably 30 sec or more and 15 min or less, still even more preferably 1 min or more and 5 min or less. However, as mentioned above, step (A) and hence, also step (A-6) may be performed multiple times (multiple performances).

Preferably, in any of the embodiments described herein and particularly in step (A-6), there is no upper limit for the duration, preferably for the total duration, of the administration and/or application of the conditioning energy and/or heat, and/or for the duration the conditioning energy and/or heat is administered and/or applied, preferably by using the energizing means and/or heating means, as long as no burning lesions and/or damaging of skin like burning, causing lesions, rupture of capillaries and/or reflexive closure of the capillaries is caused. However, usually, the duration, preferably the total duration, is 48 hours or less, preferably 24 hours or less, even more preferably 12 hours or less, still more preferably 6 hours or less, still even more preferably 3 hours or less, further preferably 1 hour or less, even further preferably 30 min or less, still further preferably 15 min or less, still even further preferably 5 min or less. Usually, the duration, preferably the total duration, is 5 sec or more, more preferably 10 sec or more, even more preferably 30 sec or more, still more preferably 1 min or more. More preferably, the duration, preferably the total duration, is in the range of 5 sec or more and 48 hours or less, even more preferably 5 sec or more and 24 hours or less, still more preferably 10 sec or more and 12 hours or less, still even more preferably 10 sec or more and 6 hours or less, further preferably 10 sec or more and 3 hours or less, even further preferably 10 sec or more and 1 hour or less, still further preferably 10 sec or more and 30 min or less, still even further preferably sec or more and 15 min or less, furthermore preferably 1 min or more and 5 min or less.

Preferably, in any of the embodiments described herein and particularly in step (A-6) the conditioning energy and/or heat may be administered and/or applied continuously and/or pulsed.

Preferably, in any of the embodiments described herein and particularly in step (A-6), the energizing means and/or heating means is suitable and/or configured to administer and/or apply the conditioning energy continuously and/or pulsed.

Preferably, in any of the embodiments described herein and particularly in step (A-6), the energizing means, heating means and/or the heat has a temperature which is 1% or more, more preferably 3% or more, even more preferably 5% or more, still more preferably 8% or more, still even more preferably 9% or more, further preferably 10% or more higher than the temperature on the skin of untreated skin, preferably when referred to ° C. (degrees Celsius), preferably when measured as described in the Materials and Methods' section ‘Thermal measurement and skin reddening’. There is no upper limit for the temperature as long as no burning lesions and/or damaging of skin like burning, causing lesions, rupture of capillaries and/or reflexive closure of the capillaries is caused. However, usually, the heating means and/or the heat has a temperature which is 90% or less, preferably 70% or less, more preferably 50% or less, still more preferably 20% or less, still even more preferably 15% or less higher than the temperature on the skin of untreated skin by. Preferably, the heating means and/or the heat has a temperature which is in the range of 1% or more and 90% or less, more preferably 3% or more and 70% or less, even more preferably by 5% or more and 70% or less, still more preferably by 8% or more and 50% or less, still even more preferably by 9% or more and 20% or less, further preferably by 10% or more and 15% or less higher than the temperature on the skin of untreated skin.

Preferably, in any of the embodiments described herein and particularly in step (A-6), the energizing means, heating means and/or the heat has a temperature which is 0.2° C. or more, more preferably 0.3° C. or more, even more preferably 0.5° C. or more, still more preferably 0.8° C. or more, still even more preferably 1° C. or more, further preferably 1.5° C. or more, even further preferably 2° C. or more, still further preferably by 3° C. or more higher than the temperature on the skin of untreated skin, preferably when measured as described in the Materials and Methods' section ‘Thermal measurement and skin reddening’. There is no upper limit for the temperature as long as no burning lesions and/or damaging of skin is caused. However, usually, the heating means and/or the heat has a temperature which is 30° C. or less, preferably 25° C. or less, even more preferably 20° C. or less, still more preferably 15° C. or less, still even more preferably 8° C. or less, further preferably 5° C. or less higher than the temperature on the skin of untreated skin of Preferably, the heating means and/or the heat has a temperature in the range of 0.2° C. or more and 30° C. or less, more preferably 0.3° C. or more and 30° C. or less, even more preferably by 0.5° C. or more and 25° C. or less, still more preferably by 0.8° C. or more and 25° C. or less, still even more preferably by 1° C. or more and 20° C. or less, further preferably by 1.5° C. or more and 15° C. or less, even further preferably by 2° C. or more and 8° C. or less, still further preferably by 3° C. or more and 5° C. or less higher that than the temperature on the skin of untreated skin of.

Preferably, in any of the embodiments described herein and particularly in step (A-6), the energizing means, heating means and/or the heat has a temperature of 25° C. or more, more preferably 27° C. or more, even more preferably 28° C. or more, still more preferably 29° C. or more, still even more preferably 30° C. or more, further preferably 31° C. or more, even further preferably 34° C. or more, still further preferably 36° C. or more, still even further preferably 37° C. or more, furthermore preferably 38° C. or more, even furthermore preferably 40° C. or more, preferably when measured as described in the Materials and Methods' section ‘Thermal measurement and skin reddening’. There is no upper limit for the temperature as long as no burning lesions and/or damaging of skin like burning, causing lesions, rupture of capillaries and/or reflexive closure of the capillaries is caused. However, usually, the heating means and/or the heat has a temperature of 65° C. or less, preferably 58° C. or less, more preferably 45° C. or less, still more preferably 42° C. or less, still even more preferably 41° C. or less. More preferably, the heating means and/or the heat has a temperature in the range 26° C. or more and 65° C. or less, more preferably 28° C. or more and 65° C. or less, even more preferably 29° C. or more and 58° C. or less, still more preferably 30° C. or more and 58° C. or less, still even more preferably 31° C. or more and 58° C. or less, further preferably 34° C. or more and 50° C. or less, even further preferably 36° C. or more and 50° C. or less, still further preferably 37° C. or more and 45° C. or less, still even further preferably 38° C. or more and 42° C. or less, furthermore preferably 40° C. or more and 41° C. or less.

Preferably, in step (A-6), the skin is of a skin area, preferably of a skin area [a], and the untreated skin is of an untreated skin area, wherein the untreated skin area is distanced apart from the skin area by 1 cm or more and 10 cm or less. The distance is preferably measured from a point on the outline of the skin area to a point on the outline of the untreated skin area, wherein the points with the smallest distance to each other are taken. Preferably, the untreated skin is of an arm or leg, more preferably a human. Typically, untreated skin has a temperature, preferably a temperature on the skin, of 36° C. or less, more typically in the range of 20° C. or more and 34° C. or less, even more typically 22° C. or more and 34° C. or less, still more typically 24° C. or more and 34° C. or less, still even more typically 24° C. or more and 32° C. or less, preferably when measured as described in the Materials and Methods' section ‘Thermal measurement and skin reddening’.

Preferably, in any of the embodiments described herein and particularly in step (A-6), the temperature on the skin may be measured by any suitable method known to the person skilled in the art, preferably it is measured as described in the Materials and Methods' section under the topic ‘Thermal measurement and skin reddening’.

Preferably, in case the conditioning energy is a negative pressure, the negative pressure is a pressure below atmosphere pressure. Preferably, in case the conditioning energy is a negative pressure, the negative pressure is to an extent in the range of 0 hPa (hectopascale(s)) to 1,000 hPa, more preferably 0 hPa to 600 hPa.

Preferably, in case the conditioning energy is a negative pressure, the negative pressure is 1,000 hPa or less, more preferably 600 hPa or less. Preferably, a lower limit for the negative pressure is 0 hPa, more preferably 10 hPa. More preferably, the negative pressure is in the range of 0 hPa to 1,000 hPa, more preferably 10 hPa to 600 hPa.

Preferably, in any of the embodiments described herein and particularly in step (A-6), the conditioning energy, preferably the heat, is administered to the skin by topical application.

Preferably, in any of the embodiments described herein and particularly in step (A-6), the conditioning energy, preferably the heat, is administered to the skin of the skin area. More preferably, the conditioning energy is administered to the skin of the skin area, wherein an effect thereof is generated within or on the skin of the skin area.

More preferably, the conditioning energy is administered to the skin of the skin area by applying the conditioning energy to the skin of the application area.

Even more preferably, the conditioning energy is administered to the skin of the skin area by applying the conditioning energy to the skin of the application area, wherein an effect thereof is generated within or on the skin of the skin area.

Preferably, in any of the embodiments described herein and particularly in step (A-6), the conditioning energy, preferably the heat, is applied to the skin of the application area.

More preferably, the conditioning energy is applied to the skin of the application area, wherein an effect thereof is generated within or on the skin of the skin area.

Preferably, in any of the embodiments described herein and particularly in step (A-6), the conditioning energy, preferably the heat, is applied to the skin of the application area by topical application.

Preferably, in any of the embodiments described herein and particularly in step (A-6), the conditioning energy, preferably the heat, is applied to the skin of the application area by topical application.

Preferably, in any of the embodiments described herein and particularly in step (A-6), the effect of administering and/or applying the conditioning energy, preferably the heat, is the generating of the accumulation of PBMCs, vasodilation, increased blood volume, increased sO₂, increased rHb, increased temperature and/or redness, preferably the increased temperature as mentioned in any of the embodiments described herein.

Preferably, in any of the embodiments described herein and particularly in step (A-6), the conditioning energy, preferably the heat, is applied by topical application.

Preferably, in any of the embodiments described herein and particularly in step (A-6), the application area is that of the respective skin area.

More preferably, the skin area is the skin area [a] and/or the application area is the application area [a].

Preferably, in step (A-6), the skin area and the skin area [a] are the same skin area and skin area [a], respectively, as mentioned in any of the embodiments described herein. If not mentioned otherwise, it is to be understood that any embodiment or definition described herein in terms of step (A) and a skin area or skin area [a] is independently and mutatis mutandis applicable to the skin area and the skin area [a], respectively, of step (A-6).

Preferably, in step (A-6), the application area and the application area [a] are the same application area and application area [a], respectively, as mentioned in any of the embodiments described herein. If not mentioned otherwise, it is to be understood that any embodiment or definition described herein in terms of step (A) and an application area or application area [a] is independently and mutatis mutandis applicable to the application area and the application area [a], respectively, of step (A-6).

Preferably, in any of the embodiments described herein and particularly in step (A-6), the administering of the conditioning energy to the skin of the subject is an applying of the conditioning energy to the skin of the subject.

If not mentioned otherwise, it is to be understood that any embodiment or definition of the conditioning energy and/or heat described in step (A-6) is independently and mutatis mutandis applicable to the conditioning energy and/or heat as mentioned in any of the embodiments described herein, particularly in any of the embodiments of the topical dosage forms and/or the medical devices described herein.

If not mentioned otherwise, it is to be understood that any embodiment or definition of the energizing means and/or heating means described in step (A-6) is independently and mutatis mutandis applicable to the energizing constituent and/or heating constituent, respectively, as mentioned in any of the embodiments described herein, particularly in any of the embodiments of the topical dosage forms and/or the medical devices described herein, wherein for that reason, where applicable, the term ‘energizing means’ is to be understood ‘energizing constituent’ and the term ‘heating means’ is to be understood ‘heating constituent’.

In any of the embodiments described herein, the expressions “temperature on the skin”, “temperature of the skin” and/or “skin surface temperature” may be used interchangeably. Similarly, in any of the embodiments described herein, the expressions “increased temperature on the skin”, “increased temperature of the skin” and/or “increased skin surface temperature” may be used interchangeably.

It is to be understood that any of the embodiments mentioned under the outline note ‘As further regards step (A-6) for any of the embodiments as described herein’ is independently applicable and/or combinable with any of the embodiments for the method as described herein and particularly with any embodiment of step (A) of the method as described herein.

As further regards step (A-7) for any of the embodiments as described herein

As stated above, step (A-7) requires administering a skin-conditioning agent to the skin.

Preferably, in any of the embodiments described herein and particularly in step (A-7), the skin-conditioning agent does not cause an allergic reaction in the subject.

Preferably, in any of the embodiments described herein and particularly in step (A-7), the skin-conditioning agent is administered in an amount and/or for a duration suitable to achieve the beneficial effects, is pharmaceutically acceptable and does not cause lesions and/or a damaging of skin like burning, causing lesions, rupture of capillaries and/or reflexive closure of the capillaries.

Preferably, in any of the embodiments described herein and particularly in step (A-7), the skin-conditioning agent may be administered to the skin in any amount and/or for any duration as long as it is pharmaceutically acceptable and does not cause lesions and/or a damaging of skin like burning, causing lesions, rupture of capillaries and/or reflexive closure of the capillaries.

The skin-conditioning agent as mentioned in any of the embodiments described herein and particularly in step (A-7), may be, preferably is any type or kind of substance(s), composition or formulation suitable for modifying a condition of the skin particularly in respect of the amount of PBMCs, the vasodilation of the capillaries within the skin, the blood volume within the skin, the sO₂ within the skin, the rHb within the skin, the temperature on the skin and/or the redness on the skin.

More preferably, in any of the embodiments described herein and particularly in step (A-7), the skin-conditioning agent:
is administered to generate, preferably is administered in an amount and/or for a duration is suitable and/or sufficient to generate; and/or
is suitable and/or sufficient to generate and/or generates:
   an accumulation of PBMCs within the skin, wherein it is to be understood that preferably any embodiment or definition thereof which is described under the outline note ‘As further regards step (A-0) for any of the embodiments as described herein’ is independently and mutatis mutandis applicable; and/or
   a vasodilation of the capillaries within the skin, wherein it is to be understood that preferably any embodiment or definition thereof which is described under the outline note ‘As further regards step (A-1) for any of the embodiments as described herein’ is independently and mutatis mutandis applicable; and/or
   an increased blood volume within the skin, wherein it is to be understood that preferably any embodiment or definition thereof which is described under the outline note ‘As further regards step (A-2) for any of the embodiments as described herein’ is independently and mutatis mutandis applicable; and/or
   an increased sO₂ and/or an increased rHb within the skin, wherein it is to be understood that preferably any embodiment or definition thereof which is described under the outline note ‘As further regards step (A-3) for any of the embodiments as described herein’ is independently and mutatis mutandis applicable; and/or
   an increased temperature on the skin, wherein it is to be understood that preferably any embodiment or definition thereof which is described under the outline note ‘As further regards step (A-4) for any of the embodiments as described herein’ is independently and mutatis mutandis applicable; and/or
   a redness on the skin, wherein it is to be understood that preferably any embodiment or definition thereof which is described under the outline note ‘As further regards step (A-5) for any of the embodiments as described herein’ is independently and mutatis mutandis applicable,
even more preferably,
   the vasodilation of the capillaries within the skin,
   the increased blood volume within the skin,
   the increased sO₂ and/or the increased rHb within the skin, and/or
   the increased temperature on the skin,
still more preferably,
   the increased blood volume within the skin,
   the increased sO₂ and/or the increased rHb within the skin, and/or
   the increased temperature on the skin,
still even more preferably
   the increased sO₂ and/or the increased rHb within the skin, and/or
   the increased temperature on the skin.

In any of the embodiments described herein and particularly in step (A-7), the skin-conditioning agent may be any blood-circulation-increasing agent, vasodilating agent, skin-temperature increasing agent, skin-502-increasing agent and/or skin-rHb-increasing agent. Examples for the skin-conditioning agent comprise nitrates, alpha blockers, ACE-inhibitors, ginkgo preparations like gingko balm, calcium antagonists, dihydralazine, minoxidil, dihydroergotoxin, nicotinic acid analogues, vasodilators like methylnicotinat, moxa herbs, moxa herbs, capsaicine, and/or pentoxifylline, a heat crème, a vasodilator containing crème, a methylnicotinat containing crème, particularly Kytta® heat balm (Kytta® heat balm containing methylnicotinat [“Kytta® heat balm” by P&G Health Germany GmbH, Germany, PZN 12358936]) or any combination thereof. Preferably, the skin-conditioning agent comprises as active ingredient(s), preferably consists of as active ingredient(s) of, nitrates, alpha blockers, ACE-inhibitors, ginkgo preparations like gingko balm, calcium antagonists, dihydralazine, minoxidil, dihydroergotoxin, nicotinic acid analogues, vasodilators like methylnicotinat, moxa herbs, capsaicine, pentoxifylline, vasodilator containing crème, methylnicotinat containing crème, Kytta® heat balm or any combination thereof, more preferably methylnicotinat containing crème and/or methylnicotinat, even more preferably Kytta® heat balm and/or methylnicotinat.

Preferably, Kytta® heat balm is administered by topical application to the skin in an amount of 5 mg to 10,000 mg, more preferably 10 mg to 1,000 mg, even more preferably 15 mg to 500 mg. Hence, preferably the methylnicotinat containing crème and/or the methylnicotinat is administered in an amount corresponding to an amount of 5 mg to 10,000 mg, more preferably 10 mg to 1,000 mg, even more preferably 15 mg to 500 mg Kytta® crème.

Preferably, in any of the embodiments described herein and particularly in step (A-7), the skin-conditioning agent, preferably a vasodilator like methylnicotinat, is administered by topical application to the skin (methylnicotinate (MN) as described by Elawa et al. (Elawa S, Mirdell R, Farnebo S, Tesselaar E. Skin blood flow response to topically applied methyl nicotinate: Possible mechanisms. Skin Res Technol. 2020 May; 26(3):343-348. Epub 2019 Nov. 27. PMID: 31777124.) Particularly, the authors described that a dose of 50 μl (microliter(s)) of 20 mmol (millimole(s))/1 MN results in a reproducible perfusion response in healthy test subjects using a Laser Speckle Contrast Imager (PeriCam PSI System; Perimed AB) was used to measure the perfusion of the skin. Hence, more preferably, the skin-conditioning agent, preferably a vasodilator like methylnicotinat, is administered by topical application to the skin in a range of 10 μl to 5 ml of 20 mmol/1 MN, even more preferably 15 μl to 1 ml of 20 mmol/1 MN, still more preferably 20 μl to 500 μl of 20 mmol/1 MN.

Preferably, in any of the embodiments described herein and particularly in step (A-7), the skin-conditioning agent is in the form of any of the pharmaceutical compositions, the injectable dosage forms, the topical dosage forms, medical devices and/or the kits of parts according to the present invention or as mentioned in any of the embodiments described herein.

Preferably, the blood-circulation-increasing agent is an agent suitable for increasing and/or increasing the blood volume within the skin, i.e. a blood-volume increasing agent. Even more preferably, the blood-circulation-increasing agent is a blood-volume increasing agent.

Preferably, in any of the embodiments described herein and particularly in step (A-7), there is no upper limit for the duration of the application and/or administration of the skin-conditioning agent as long as no burning lesions and/or damaging of skin like burning, causing lesions, rupture of capillaries and/or reflexive closure of the capillaries is caused. However, usually, the skin-conditioning agent is applied and/or administered for a duration of 60 min or less, more preferably 30 min or less, even more preferably 15 min or less, still more preferably 5 min or less, still even more preferably 2 min or less. Usually, the skin-conditioning agent is applied and/or administered for a duration of 5 sec or more, more preferably 10 sec or more, even more preferably 30 sec or more, still more preferably 1 min or more. More preferably, the skin-conditioning agent is applied and/or administered for a duration in the range of 5 sec or more and 60 min or less, more preferably 10 sec or more and 30 min or less, even more preferably 30 sec or more and 15 min or less, still more preferably 1 min or more and 5 min or less.

Preferably, in any of the embodiments described herein and particularly in step (A-7), the administering of the skin-conditioning agent to the skin may be, preferably, is accomplished by topical application and/or by injection, more preferably by topical application.

In case of an administration by topical application, this preferably placed on the back, belly, buttocks, upper arm, forearm and/or upper leg more preferably the upper arm, forearm and/or upper leg.

In case of an injection, the injection is preferably into a sub-topical layer of the skin, more preferably into the epidermis, dermis and/or subcutis, even more preferably the dermis, of the skin. The injection is preferably placed 6 mm or less and 0.05 mm or more, more preferably 4 mm or less and 0.3 mm or more, even more preferably 3 mm or less and 0.5 mm or more in skin thickness direction into the skin, preferably when measured from the skin surface towards the bones. Preferably, the injection is placed into the back, belly, buttocks, upper arm, forearm and/or upper leg more preferably the upper arm, forearm and/or upper leg.

Preferably, for the injection any of the pharmaceutical compositions, injectable dosage form and/or kits of parts as mentioned in any of the embodiments described herein and which is/are suitable and/or configured for injection and which comprise(s) the skin-conditioning agent is used.

Preferably, for the administration by topical application any of the pharmaceutical compositions, topical dosage form and/or kits of parts as mentioned in any of the embodiments described herein and which is/are suitable and/or configured for an administration by topical application and which comprise(s) the skin-conditioning agent is used.

Preferably, in any of the embodiments described herein and particularly in step (A-7), the skin-conditioning agent is administered to the skin by topical application and/or by injection, preferably by topical application.

Preferably, in any of the embodiments described herein and particularly in step (A-7), the skin-conditioning agent is administered to the skin of the skin area.

More preferably, the skin-conditioning agent is administered to the skin of the skin area, wherein an effect thereof is generated within or on the skin of the skin area.

More preferably, the skin-conditioning agent is administered to the skin of the skin area by applying the skin-conditioning agent to the skin of the application area.

Even more preferably, the skin-conditioning agent is administered to the skin of the skin area by applying the skin-conditioning agent to the skin of the application area, wherein an effect thereof is generated within or on the skin of the skin area.

Preferably, in any of the embodiments described herein and particularly in step (A-7), the skin-conditioning agent is applied to the skin of the application area.

More preferably, the skin-conditioning agent is applied to the skin of the application area, wherein an effect thereof is generated within or on the skin of the skin area.

Preferably, in any of the embodiments described herein and particularly in step (A-7), the skin-conditioning agent is applied to the skin of the application area by topical application and/or by injection, preferably by topical application.

Preferably, in any of the embodiments described herein and particularly in step (A-7), the effect of administering and/or applying of the skin-conditioning agent is the generating of the accumulation of PBMCs, vasodilation, increased blood volume, increased sO₂, increased rHb, increased temperature and/or redness, preferably the increased blood volume, increased sO₂ and/or increased rHb as mentioned in any of the embodiments described herein.

Preferably, in any of the embodiments described herein and particularly in step (A-7), the skin-conditioning agent is applied by topical application.

Preferably, in step (A-7) in any of the embodiments described herein, the application area is that of the respective skin area.

More preferably, the skin area is the skin area [a] and/or the application area is the application area [a].

Preferably, in step (A-7) in any of the embodiments described herein, the skin area and the skin area [a] are the same skin area and skin area [a], respectively, as mentioned in any of the embodiments described herein. If not mentioned otherwise, it is to be understood that any embodiment or definition described herein in terms of step (A) and a skin area or skin area [a] is independently and mutatis mutandis applicable to the skin area and the skin area [a], respectively, of step (A-7).

Preferably, in step (A-7) in any of the embodiments described herein, the application area and the application area [a] are the same application area and application area [a], respectively, as mentioned in any of the embodiments described herein. If not mentioned otherwise, it is to be understood that any embodiment or definition described herein in terms of step (A) and an application area or application area [a] is independently and mutatis mutandis applicable to the application area and the application area [a], respectively, of step (A-7).

It is to be understood that any of the embodiments mentioned under the outline note ‘As further regards step (A-7) for any of the embodiments as described herein’ is independently applicable and/or combinable with any of the embodiments for the method as described herein and particularly with any embodiment of step (A) of the method as described herein.

As further regards step (A-8) for any of the embodiments as described herein

As stated above, step (A-8) requires administering PBMCs to the skin the skin of the subject.

Preferably, in any of the embodiments described herein and particularly in step (A-8), the PBMCs are administered into the tissue of the skin of the subject.

Preferably, in any of the embodiments described herein and particularly step (A-8) requires injecting the PBMCs into the skin of the subject. More preferably, in step (A-8), the PBMCs are injected into the tissue of the skin of the subject.

More preferably, in any of the embodiments described herein and particularly in step (A-8), the PBMCs are administered, applied and/or injected into a sub-topical layer of the skin, more preferably into the epidermis, dermis and/or subcutis, even more preferably the dermis, of the skin. Even more preferably into the tissue of a sub-topical layer of the skin, more preferably into the tissue of the epidermis, dermis and/or subcutis, even more preferably the dermis, of the skin.

Preferably, in step (A-8) in any of the embodiments described herein, the injection is into a sub-topical layer of the skin, more preferably into the epidermis, dermis and/or subcutis, even more preferably the dermis, of the skin. The injection is preferably placed 6 mm or less and 0.05 mm or more, more preferably 4 mm or less and 0.5 mm or more, even more preferably 3 mm or less and 0.5 mm or more in skin thickness direction into the skin, preferably when measured from the skin surface towards the bones. Preferably, the injection is placed into the back, belly, buttocks, upper arm, forearm and/or upper leg more preferably the upper arm, forearm and/or upper leg.

Preferably, in any of the embodiments described herein and particularly in step (A-8), the PBMCs are blood-derived PBMCs.

Preferably, in any of the embodiments described herein and particularly in step (A-8), the PBMCs administered are isolated PBMCs.

The PBMCs may be, preferably, are prepared and formulated by any suitable method known to the person skilled in the art, preferably as described in the Materials and Methods' section ‘Preparation of PBMCs’. The PBMCs may be formulated in any kind of formulation which is pharmaceutical acceptable and suitable for injection, preferably injection into the subject, as long as the PBMCs are maintained alive or at least an effective amount of PBMCs are maintained alive.

The PBMCs may by freshly prepared or they may have been frozen, preferably in liquid nitrogen, and thawed and reconstituted prior to use in media and/or solutions like pharmaceutical acceptable media and solutions, in order to obtain the formulation which is pharmaceutical acceptable and suitable for injection. Preferably, the PBMCs may be, preferably, are prepared and formulated as described in the Materials and Methods' section ‘Preparation of PBMCs’.

Preferably, in any of the embodiments described herein and particularly in step (A-8), the PBMCs:
are administered, applied and/or injected to generate, preferably are administered, applied and/or injected in an amount is suitable and/or sufficient to generate; and/or
are suitable to generate and/or generates:
   an accumulation of PBMCs within the skin, wherein it is to be understood that preferably any embodiment or definition thereof which is described under the outline note ‘As further regards step (A-0) for any of the embodiments as described herein’ is independently and mutatis mutandis applicable.
   wherein it is to be understood that preferably any embodiment or definition thereof which is described under the outline note mutatis mutandis

Preferably, in any of the embodiments described herein and particularly in step (A-8), the PBMCs are administered, applied and/or injected in an amount of 1×10⁵ PBMCs (100,000 PBMCs) or more, more preferably 5×10⁵ PBMCs (500,000 PBMCs) or more, even more preferably 1×10⁶ PBMCs (1 million PBMCs) or more, still more preferably 2×10⁶ PBMCs (2 million PBMCs) or more, still even more preferably 4×10⁶ PBMCs (4 million PBMCs) or more per injection, preferably per injection dose. There is no upper limit for the amount of PBMCs, however, usually, an upper limit may be, preferably, is 30×10⁷ PBMCs (300 million PBMCs) or less, more preferably 20×10⁷ PBMCs (200 million PBMCs) or less, even more preferably 10×10⁷ PBMCs (100 million PBMCs) or less, still more preferably 5×10⁷ PBMCs (50 million PBMCs) or less, still even more preferably 1×10⁷ PBMCs (10 million PBMCs) or less per injection, preferably per injection dose. Preferably, the PBMCs are administered, applied and/or injected in an amount in the range of 1×10⁵ PBMCs or more and 30×10⁷ PBMCs or less, more preferably 5×10⁵ PBMCs or more and 20=10⁷ PBMCs or less, even more preferably 1×10⁶ PBMCs or more and 10×10⁷ PBMCs or less, still more preferably 2×10⁶ PBMCs and 5×10⁷ PBMCs or less per injection, still even more preferably 4×10⁶ PBMCs or more 1×10⁷ PBMCs or more per injection dose.

Preferably, in any of the embodiments described herein and particularly in step (A-8), the PBMCs are administered, applied and/or injected in a total amount of 1×10⁵ PBMCs or more, more preferably 5×10⁵ PBMCs or more, even more preferably 1×10⁶ PBMCs or more, still more preferably 2×10⁶ PBMCs or more, still even more preferably 4×10⁶ PBMCs or more. There is no upper limit for the total amount of PBMCs, however, usually, an upper limit may be, preferably, is 30×10⁷ PBMCs or less, more preferably 20×10⁷ PBMCs or less, even more preferably 10×10⁷ PBMCs, still more preferably 5×10⁷ PBMCs or less, still even more preferably 2×10⁷ PBMCs (20 million PBMCs) or less. Preferably, the PBMCs are administered, applied and/or injected in a total amount in the range of 1×10⁵ PBMCs or more and 30×10⁷ PBMCs or less, more preferably 5×10⁵ PBMCs or more and 20×10⁷ PBMCs or less, even more preferably 1×10⁶ PBMCs or more and 10×10⁷ PBMCs or less, still more preferably 2×10⁶ PBMCs and 5×10⁷ PBMCs or less per injection, still even more preferably 4×10⁶ PBMCs or more 2×10⁷ PBMCs or more.

Preferably, in any of the embodiments described herein and particularly in step (A-8), the PBMCs are administered, applied and/or injected into a sub-topical layer of the skin, more preferably, into the epidermis, dermis and/or subcutis, even more preferably the dermis, and/or the injection is preferably placed 6 mm or less and 0.05 mm or more, more preferably 4 mm or less and 0.3 mm or more, even more preferably 3 mm or less and 0.5 mm or more in skin thickness direction, preferably when measured from the skin surface towards the bones. Preferably, the injection is placed into the back, belly, buttocks, upper arm, forearm and/or upper leg more preferably the upper arm, forearm and/or upper leg.

Preferably, in any of the embodiments described herein and particularly in step (A-8), the PBMCs are administered to the skin by injection.

Preferably, in any of the embodiments described herein and particularly in step (A-8), the PBMCs are administered to the skin of the skin area.

More preferably, the PBMCs are administered to the skin of the skin area, wherein an accumulation of PBMCs as mentioned in any of the embodiments described herein is generated within the skin of the skin area.

More preferably, the PBMCs are administered to the skin of the skin area by applying the PBMCs into the skin of the application area.

Even more preferably, the PBMCs are administered to the skin of the skin area by applying the PBMCs into the skin of the application area, wherein an accumulation of PBMCs as mentioned in any of the embodiments described herein is generated within the skin of the skin area.

Preferably, in any of the embodiments described herein and particularly in step (A-8) in any of the embodiments described herein, the PBMCs are applied into the skin of the application area.

More preferably, in step (A-8) in any of the embodiments described herein, the PBMCs are applied into the skin of the application area by injection.

More preferably, the PBMCs are applied into the skin of the application area, wherein an accumulation of PBMCs as mentioned in any of the embodiments described herein is generated within the skin of the skin area.

Preferably, in step (A-8) in any of the embodiments described herein, the PBMCs are applied by injection.

Preferably, in step (A-8) in any of the embodiments described herein, the application area is that of the respective skin area.

More preferably, in step (A-8), the skin area is the skin area [a] and/or the application area is the application area [a].

Preferably, in step (A-8) in any of the embodiments described herein, the skin area and the skin area [a] are the same skin area and skin area [a], respectively, as mentioned in any of the embodiments described herein. If not mentioned otherwise, it is to be understood that any embodiment or definition described herein in terms of step (A) and a skin area or skin area [a] is independently and mutatis mutandis applicable to the skin area and the skin area [a], respectively, of step (A-8).

Preferably, in step (A-8) in any of the embodiments described herein, the application area and the application area [a] are the same application area and application area [a], respectively, as mentioned in any of the embodiments described herein. If not mentioned otherwise, it is to be understood that any embodiment or definition described herein in terms of step (A) and an application area or application area [a] is independently and mutatis mutandis applicable to the application area and the application area [a], respectively, of step (A-8).

Preferably, in step (A-8) in any of the embodiments described herein, the administering of the PBMCs to the skin of the subject is an injection of the PBMCs into the skin of the subject.

Preferably, in any of the embodiments described herein and particularly in step (A-8), in any of the embodiments described herein, the PBMCs are defined and obtained as described above. More preferably, the PBMCs are blood PBMCs and/or isolated PBMCs, even more preferably isolated PBMCs. More preferably, the PBMCs are lymphocytes, more preferably naïve PBMCs, even more preferably naïve lymphocytes, still more preferably naïve B-cells and/or naïve T-cells, still even more preferably naïve T-cells.

Preferably, in any of the embodiments described herein and particularly in step (A-8), in any of the embodiments described herein, the PBMCs are not subjected to culturing and/or incubation externally of the subject's body.

More preferably, in any of the embodiments described herein and particularly in step (A-8), the PBMCs are not subjected to culturing and/or incubation externally of the subject's body:
for an effective duration; and/or
for 1 hour or more, even more preferably 12 hour or more, still more preferably 1 day or more, still even more preferably 3 day or more, further preferably 7 days or more and 4 weeks or less, preferably 2 weeks or less; and/or
at 35° C. or more, preferably at 36° C. or more, even more preferably at 37° C. or more and preferably 50° C. of less; and/or
using a cell incubator for an effective duration; and/or
under an atmosphere having an CO₂-content increased in comparison to air, preferably under a CO₂ atmosphere of 1% CO₂ or more, more preferably a CO₂ atmosphere of 5% CO₂ or more; and/or
in the presence of TGF-β (transforming growth factor β), for instance in an amount of about 50 pg/ml TGF-β or less (picograms/millilitre), of IL-6 (interleukin-6), of IL-7 (interleukin-7), of IL-15 (interleukin-15), of CD3 (cluster of differentiation 3) and/or of CD28 (cluster of differentiation 28) in an effective amount; and/or
with any cell culturing medium, like serum-free or serum-containing medium, in effective amounts.

This does not does not exclude freezing of PBMCs prior to administration.

Hence, in any of the embodiments described herein and particularly in step (A-8), the PBMCs may have been frozen prior to administration, preferably at −165° C. or more, more preferably in liquid nitrogen

Furthermore, this does not exclude premixing of the PBMCs with one or more of the immunomodulatory substance(s) prior to administration, preferably 24 hours or less, more preferably 12 hours or less, even more preferably 6 hours or less, still more preferably 3 hours or less, still even more preferably 1.5 hours or less prior to administration.

Hence, in any of the embodiments described herein and particularly in step (A-8), the PBMCs may be mixed with one or more of the immunomodulatory substance(s) prior to administration, preferably 24 hours or less, more preferably 12 hours or less, even more preferably 6 hours or less, still more preferably 3 hours or less, still even more preferably 1.5 hours or less prior to administration.

Preferably, in any of the embodiments described herein and particularly in step (A-8), the PBMCs are handled prior to administration at a temperature of not more than 35.5° C., more preferably 35.5° C. or less, even more preferably at a temperature of 34° C. or less, still more preferably at a temperature of 32° C. or less, still even more preferably at a temperature 30° C. or less, further preferably at a temperature 28° C. or less and preferably −195° C. or more, preferably under normal air conditions.

It is to be understood that any of the embodiments mentioned under the outline note ‘As further regards step (A-8) for any of the embodiments as described herein’ is independently applicable and/or combinable with any of the embodiments for the method as described herein and particularly with any embodiment of step (A) of the method as described herein.

The substances and agents as mentioned in any of the embodiments described herein, particularly in the embodiments of the method described herein, are usually formulated into compositions like pharmaceutical compositions, and dosage forms like topical dosage forms and/or injectable dosage forms.

Hence, in a further aspect, the present invention relates to compositions, dosage forms, medical devices and kits of parts comprising, preferably consisting of, one or more of the substances and/or agents as mentioned in any of the embodiments described herein as an active ingredient(s), optionally together with a pharmaceutically acceptable vehicle, diluent, adjuvant, excipient, carrier, additive and/or salt.

If not mentioned otherwise, it is to be understood that any embodiment or definition described herein in terms of the substances and/or agents, particularly in terms of the immunomodulatory substance(s) and/or the skin-conditioning agent, their respective uses thereof and/or their respective medical uses thereof, is independently and mutatis mutandis applicable to all of the pharmaceutical compositions, dosage forms, medical devices and/or kits of parts.

The present invention thus relates to pharmaceutical compositions, injectable dosage forms, topical dosage forms, medical devices and/or kits of parts comprising, preferably consisting of, an immunomodulatory substance(s) and/or skin-conditioning agent as active ingredient(s).

Further, the present invention relates to pharmaceutical compositions, injectable dosage forms, topical dosage forms, medical devices and/or kits of parts comprising, preferably consisting of, an immunomodulatory substance(s) as active ingredient(s). Preferably, the pharmaceutical compositions, the injectable dosage forms, the topical dosage forms, the medical devices and/or the kits of parts further comprise, preferably consist of, a skin-conditioning agent as further active ingredient(s).

Furthermore, the present invention relates to pharmaceutical compositions, injectable dosage forms, topical dosage forms, medical devices and/or kits of parts comprising, preferably consisting of, a skin-conditioning agent as active ingredient(s). Preferably, the pharmaceutical compositions, the injectable dosage forms, the topical dosage forms, the medical devices and/or the kits of parts further comprise, preferably consist of, an immunomodulatory substance(s) as further active ingredient(s).

Moreover, the present invention relates to the pharmaceutical compositions, the injectable dosage forms, the topical dosage forms, the medical devices and/or the kits of parts for use as a medicament.

Moreover, the present invention relates to the pharmaceutical compositions, the injectable dosage forms, the topical dosage forms, the medical devices and/or the kits of parts which are configured and/or suitable for use in the method as mentioned in any of the embodiments described herein, that is the method for treating and/or preventing an inflammatory disease, immunological disease and/or autoimmunological disease in a subject as mentioned in any of the embodiments described herein. Hence, the pharmaceutical compositions, the injectable dosage forms, the topical dosage forms, the medical devices and/or the kits of parts which may be configured and/or suitable for use in at least parts of the steps or all steps of the method as mentioned in any of the embodiments described herein,

Moreover, the present invention relates to the pharmaceutical compositions, the injectable dosage forms, the topical dosage forms, the medical devices and/or the kit of parts for use in the method described herein that is the method for treating and/or preventing an inflammatory disease, immunological disease and/or autoimmunological disease in a subject.

The present invention further relates to the use of the pharmaceutical compositions and/or the injectable dosage forms for producing an article or a medical device as mentioned in any of the embodiments described herein.

The substance(s) like the immunomodulatory substance(s), cytokine-like acting substance, IFN-γ-like acting substance, IL-4-like acting substance, BDNF-like acting substance, IL-2-like acting substance, IFN-γ, IL-4, BDNF and/or IL-2 may be administered, applied and/or delivered by any suitable route to the skin. Similarly, the skin-conditioning agent(s) may be administered, applied and/or delivered by any suitable route to the skin. Preferably, the substance(s) and/or skin-conditioning agent(s) are administered, applied and/or delivered topically or by injection, for instance as described herein. Hence, the substance(s) and/or skin-conditioning agent(s) are preferably suitable and/or prepared for a topical administration, application and/or delivery and/or they are suitable and/or prepared for an administration, application and/or delivery by injection, for instance as described herein.

The substance(s) and/or the skin-conditioning agent(s) may be formulated or are in any form known to the person skilled in the art which is suitable for an administration, application and/or delivery of these substance(s) and/or the skin-conditioning agent(s) to the skin.

For example, the substance(s) and/or the skin-conditioning agent(s) may be provided, formulated or are independently in any form selected from an ointment, emulsion, solution, suspension, paste, gel, lotion, tincture, particulate material, powder, lyophilisate, liquid, dry preparation or solid preparations for an application to the skin or for injection, slow-release formulation, wadding, tamponade, crème, balm, foam, gel, spray, stick, liquid plaster, spray plaster, plaster for intradermal administration, time-release plaster, transdermal patch, plaster, microneedle patch, patch, pad, wadding, padding, dressing, compress, bandage, wherein any of these are optionally of a multi-compartment type as mentioned in any of the embodiments described herein, injection solution, injection gel, preparation for intraepidermal, intradermal and/or subcutaneous injection, a dermatological preparation, preferably in the form of an application for external use or injection, cartridge for an injection pen or the medical device as described herein, carpule, vial, ampule, prefilled injection pen, prefilled syringe, prefilled multi-channel syringe, a medical device, wherein any of these is optionally of a multi-channel or multi-chamber type as mentioned in any of the embodiments described herein or any combination thereof, and/or they are in the form of the injectable dosage forms, the topical dosages forms, the medical devices and/or the kits of parts as mentioned in any of the embodiments described herein.

Preferably, the substance(s) and/or skin-conditioning agent(s) are independently provided, formulated and/or in the form of any of the pharmaceutical compositions (I), (II), (III), (IV), (X), injectable dosage forms (I), (II), (X), topical dosage forms (I), (X), medical devices (I), (X) and/or kits of parts (I), (II), (III), (IV), (V) and/or (X) as mentioned in any of the embodiments described herein.

For the sake of simplicity and clarity, the embodiments of the pharmaceutical compositions (I), (II), (III), (IV), (X), injectable dosage forms (I), (II), (X), topical dosage forms (I), (X), medical devices (I), (X) and/or kits of parts (I), (II), (III), (IV), (V) and/or (X) were often only written out in full length for the cytokine(s), interferon(s), interleukin(s), neurotrophin(s), IFN-γ, IL-4, BDNF and/or IL-2. However, if not mentioned otherwise, it is to be understood that any of these embodiments of the pharmaceutical compositions (I), (II), (III), (IV), (X), injectable dosage forms (I), (II), (X), topical dosage forms (I), (X), medical devices (I), (X) and/or kits of parts (I), (II), (III), (IV), (V) and/or (X) is independently and mutatis mutandis applicable to the cytokine-like acting substance(s), interferon-like acting substance(s), interleukin-like acting substance(s), neurotrophin-like acting substance(s), IFN-γ-like acting substance(s), IL-2-like acting substance(s), IL-4-like acting substance(s) and/or BDNF-like acting substance(s), wherein for that reason, where applicable, the term ‘cytokine(s)’ is to be understood ‘cytokine-like acting substance(s)’, ‘interferon(s)’ is to be understood ‘interferon-like acting substance(s)’, ‘interleukin(s)’ is to be understood ‘interleukin-like acting substance(s)’, ‘neurotrophin(s)’ is to be understood ‘neurotrophin-like acting substance(s)’, ‘IFN-γ’ is to be understood IFN-γ-like acting substance(s)′, ‘IL-4’ is to be understood ‘IL-4-like acting substance(s)’, ‘BDNF’ is to be understood ‘BDNF-like acting substance(s)’ and ‘IL-2’ is to be understood ‘IL-2-like acting substance(s)’. Preferably, in one particular embodiment all of such terms are replaced or all of such terms remain unchanged, however, not some replaced and some not. The embodiments referring to cytokine(s), interferon(s), neurotrophin(s), interleukin(s), IFN-γ, IL-4, BDNF and/or IL-2 are more preferred.

Furthermore, if not mentioned otherwise, it is to be understood that any of the embodiments of the pharmaceutical compositions (I), (II), (III), (IV), (X), injectable dosage forms (I), (II), (X), topical dosage forms (I), (X), medical devices (I), (X) and/or kits of parts (I), (II), (III), (IV), (V) and/or (X) referring to amounts of IFN-γ, IL-4, BDNF and/or IL-2 indicated in IU (international units) or IU/kg (international units/kilogram) body mass of the subject is independently and mutatis mutandis applicable to the IFN-γ-like acting substance(s), IL-2-like acting substance(s), IL-4-like acting substance(s) and/or BDNF-like acting substance(s), wherein for that reason, where applicable, the term ‘IFN-γ’ is to be understood ‘IFN-γ-like acting substance(s)’, ‘IL-4’ is to be understood ‘IL-4-like acting substance(s)’, ‘BDNF’ is to be understood ‘BDNF-like acting substance(s)’ and ‘IL-2’ is to be understood ‘IL-2-like acting substance(s)’. Preferably, in one particular embodiment all of such terms are replaced or all of such terms remains unchanged, however, not some replaced and some not. The embodiments referring to IFN-γ, IL-4, BDNF and/or IL-2 are more preferred.

In a general concept, the present invention relates to a composition comprising a combination of an immunomodulatory substance(s) and a skin-conditioning agent.

Hence, the present invention relates to a pharmaceutical composition (I) comprising, preferably consisting of, preferably as active ingredient(s):
a skin-conditioning agent; and
an immunomodulatory substance(s).

In a further general concept, the present invention relates to a composition comprising an immunomodulatory substance(s), wherein the composition is suitable to deliver the immunomodulatory substance(s) in a low amount.

Hence, the present invention further relates to a pharmaceutical composition (II) comprising, preferably consisting of, preferably as active ingredient(s):
an IFN-γ-like acting substance(s);
an IL-4-like acting substance(s);
an BDNF-like acting substance(s); and/or
an IL-2-like acting substance(s),
wherein the pharmaceutical composition is suitable to deliver:
IFN-γ-like acting substance(s)
in an amount equivalent to 600 IU IFN-γ/kg body mass of the subject or less;
in an amount equivalent in activity to 30 ng IFN-γ/kg body mass of the subject or less;
in a total amount equivalent to 30,000 IU IFN-γ or less; and/or in a total amount equivalent in activity to 1,500 ng IFN-γ or less;
IL-4-like acting substance(s)
in an amount equivalent in activity to 20 ng IL-4/kg body mass of the subject or less; and/or in a total amount equivalent in activity to 1,000 ng IL-4 or less;
BDNF-like acting substance(s)
in an amount equivalent in activity to 10 ng BDNF/kg per body mass of the subject or less; and/or in a total amount equivalent in activity to 500 ng BDNF or less; and/or
IL-2-like acting substance(s) in an amount equivalent to 10 IU/kg body mass of the subject or less;
in an amount of 0.6 ng/kg body mass of the subject or less;
in a total amount equivalent in activity to 500 IU IL-2 or less; and/or
in a total amount equivalent in activity to 30.5 ng IL-2 or less.

Preferably, the IFN-γ-like acting substance is IFN-γ and/or any derivative, fragment, protein-analogue, peptide, protein-variant, biopharmaceutical, inductor, precursor, prodrug, mutein, co-drug, propeptide thereof and/or any pharmaceutically acceptable salt thereof,
the IL-4-like acting substance is IL-4 and/or any derivative, fragment, protein-analogue, peptide, protein-variant, biopharmaceutical, inductor, precursor, prodrug, mutein, co-drug, propeptide thereof and/or any pharmaceutically acceptable salt thereof, the BDNF-like acting substance is BDNF and/or any derivative, fragment, protein-analogue, peptide, protein-variant, biopharmaceutical, inductor, precursor, prodrug, mutein, co-drug, propeptide thereof and/or any pharmaceutically acceptable salt thereof,
the IL-2-like acting substance is IL-2 and/or derivative, fragment, protein-analogue, peptide, protein-variant, biopharmaceutical, inductor, precursor, prodrug, mutein, co-drug, propeptide thereof and/or any pharmaceutically acceptable salt thereof, still more preferably IFN-γ, IL-4, BDNF and/or IL-2.

Hence, still more preferably, the pharmaceutical composition (II) comprises, preferably consists of, preferably as active ingredient(s):
IFN-γ;
IL-4;
BDNF; and/or
IL-2.
wherein the pharmaceutical composition (II) is suitable to deliver:
IFN-γ in an amount of 600 IU/kg body mass of the subject or less, in an amount of 30 ng/kg body mass of the subject or less, in a total amount of 30,000 IU or less and/or in a total amount of 1,500 ng or less;
IL-4 in an amount of 20 ng/kg body mass of the subject or less and/or in a total amount of 1,000 ng or less;
BDNF in an amount of 10 ng/kg per body mass of the subject or less and/or in a total amount of 500 ng or less; and/or
IL-2 in an amount of 20 IU/kg body mass of the subject or less, in an amount of 1.2 ng/kg body mass of the subject or less, in a total amount of 1,000 IU or less and/or in a total amount of 61 ng or less.

In a further general concept, the present invention relates to a composition comprising an immunomodulatory substance(s) in a low concentration.

Therefore, the present invention furthermore relates to a pharmaceutical composition (III) comprising, preferably consisting of, preferably as active ingredient(s):
an IFN-γ-like acting substance(s) in a concentration equivalent to 100,000 IU IFN-γ/ml or IU IFN-γ/g or less, or in a concentration equivalent in activity to 5,000 ng IFN-γ/ml or ng IFN-γ/g or less;
an IL-4-like acting substance(s) in a concentration or equivalent in activity to 1,000 ng IL-4/ml or ng/g or less;
an BDNF-like acting substance(s) in a concentration equivalent in activity to 1,500 ng BDNF/ml or ng/g or less; and/or
an IL-2-like acting substance(s) in a concentration equivalent to 100,000 IU IL-2/ml or IU IL-2/g less, or n a concentration equivalent in activity to 6100 ng IL-2/ml or ng IL-2/g or less.

Preferably, the IFN-γ-like acting substance is IFN-γ and/or any derivative, fragment, protein-analogue, peptide, protein-variant, biopharmaceutical, inductor, precursor, prodrug, mutein, co-drug, propeptide thereof and/or any pharmaceutically acceptable salt thereof,
the IL-4-like acting substance is IL-4 and/or any derivative, fragment, protein-analogue, peptide, protein-variant, biopharmaceutical, inductor, precursor, prodrug, mutein, co-drug, propeptide thereof and/or any pharmaceutically acceptable salt thereof, the BDNF-like acting substance is BDNF and/or any derivative, fragment, protein-analogue, peptide, protein-variant, biopharmaceutical, inductor, precursor, prodrug, mutein, co-drug, propeptide thereof and/or any pharmaceutically acceptable salt thereof,
the IL-2-like acting substance is IL-2 and/or any derivative, fragment, protein-analogue, peptide, protein-variant, biopharmaceutical, inductor, precursor, prodrug, mutein, co-drug, propeptide thereof and/or any pharmaceutically acceptable salt thereof, still more preferably IFN-γ, IL-4, BDNF and/or IL-2.

Hence, still more preferably, the pharmaceutical composition (II) comprises, preferably consists of, preferably as active ingredient(s):
IFN-γ in a concentration of 100,000 IU/ml or IU/g or less, or of 5,000 ng/ml or ng/g or less;
IL-4 in a concentration of 10,000 ng/ml or ng/g or less;
BDNF in a concentration of 10,000 ng/ml or ng/g or less; and/or
IL-2 in a concentration of 100,000 IU/ml or IU/g less, or of 6,100 ng/ml or ng/g or less.

In an even further general concept, the present invention relates to a composition comprising a combination of two or more immunomodulatory substance(s).

Hence, the present invention further relates to a pharmaceutical composition (IV) comprising, preferably consisting of, preferably as active ingredient(s):
an IFN-γ-like acting substance;
an IL-4-like acting substance;
an BDNF-like acting substance; or
an IL-2-like acting substance, and
an immunomodulatory substance(s), preferably as further active ingredient, wherein in case the pharmaceutical composition (IV) comprises, preferably consists of as active ingredient(s):
the IFN-γ-like acting substance, the immunomodulatory substance(s) is different from the IFN-γ-like acting substance;
the IL-4-like acting substance, the immunomodulatory substance(s) is different from the IL-4-like acting substance;
the BDNF-like acting substance, the immunomodulatory substance(s) is different from the BDNF-like acting substance; and
the IL-2-like acting substance, the immunomodulatory substance(s) is different from the IL-2-like acting substance.

Preferably, the IFN-γ-like acting substance is IFN-γ and/or any derivative, fragment, protein-analogue, peptide, protein-variant, biopharmaceutical, inductor, precursor, prodrug, mutein, co-drug, propeptide thereof and/or any pharmaceutically acceptable salt thereof.

Preferably, the IL-4-like acting substance is IL-4 and/or any derivative, fragment, protein-analogue, peptide, protein-variant, biopharmaceutical, inductor, precursor, prodrug, mutein, co-drug, propeptide thereof and/or any pharmaceutically acceptable salt thereof.

Preferably, the BDNF-like acting substance is BDNF and/or any derivative, fragment, protein-analogue, peptide, protein-variant, biopharmaceutical, inductor, precursor, prodrug, mutein, co-drug, propeptide thereof and/or any pharmaceutically acceptable salt thereof.

Preferably, the IL-2-like acting substance is IL-2 and/or any derivative, fragment, protein-analogue, peptide, protein-variant, biopharmaceutical, inductor, precursor, prodrug, mutein, co-drug, propeptide thereof and/or any pharmaceutically acceptable salt thereof.

More preferably, the IFN-γ-like acting substance is IFN-γ, the IL-4-like acting substance is IL-4, the BDNF-like acting substance is BDNF and the IL-2-like acting substance is IL-2.

Hence, more preferably the pharmaceutical composition (IV) comprises, preferably consists of, preferably as active ingredient(s):
IFN-γ;
IL-4;
BDNF; or
IL-2, and
an immunomodulatory substance(s), preferably as further active ingredient, wherein in case the pharmaceutical composition (IV) comprises, preferably consists of as active ingredient(s):
IFN-γ, the immunomodulatory substance(s) is different from IFN-γ;
IL-4, the immunomodulatory substance(s) is different from IL-4;
BDNF, the immunomodulatory substance(s) is different from BDNF; and
IL-2, the immunomodulatory substance(s) is different from IL-2.

In a still further general concept, the present invention relates to a composition suitable for nject on comprising a combination of two or more different immunomodulatory substance(s).

Hence, still further, the present invention relates to an injectable dosage form (I), comprising, preferably consisting of, preferably as active ingredient(s):
an IFN-γ-like acting substance;
an IL-4-like acting substance;
an BDNF-like acting substance; or
an IL-2, -like acting substance and
an immunomodulatory substance(s), preferably as further active ingredient, wherein in case of the injectable dosage form (I) comprises, preferably consists of as active ingredient(s):
IFN-γ-like acting substance, the immunomodulatory substance(s) is different from IFN-γ-like acting substance;
IL-4-like acting substance, the immunomodulatory substance(s) is different from IL-4-like acting substance;
BDNF-like acting substance, the immunomodulatory substance(s) is different from BDNF-like acting substance; and
IL-2-like acting substance, the immunomodulatory substance(s) is different from IL-2-like acting substance.

Preferably, the IFN-γ-like acting substance is IFN-γ and/or derivative, fragment, protein-analogue, peptide, protein-variant, biopharmaceutical, inductor, precursor, prodrug, mutein, co-drug, propeptide thereof and/or any pharmaceutically acceptable salt thereof.

Preferably, the IL-4-like acting substance is IL-4 and/or any derivative, fragment, protein-analogue, peptide, protein-variant, biopharmaceutical, inductor, precursor, prodrug, mutein, co-drug, propeptide thereof and/or any pharmaceutically acceptable salt thereof.

Preferably, the BDNF-like acting substance is BDNF and/or any derivative, fragment, protein-analogue, peptide, protein-variant, biopharmaceutical, inductor, precursor, prodrug, mutein, co-drug, propeptide thereof and/or any pharmaceutically acceptable salt thereof.

Preferably, the IL-2-like acting substance is IL-2 and/or any derivative, fragment, protein-analogue, peptide, protein-variant, biopharmaceutical, inductor, precursor, prodrug, mutein, co-drug, propeptide thereof and/or any pharmaceutically acceptable salt thereof.

More preferably, the IFN-γ-like acting substance is IFN-γ, the IL-4-like acting substance is IL-4, the BDNF-like acting substance is BDNF and the IL-2-like acting substance is IL-2.

Hence, more preferably the injectable dosage form (I) comprises, preferably consists of, preferably as active ingredient(s):
IFN-γ;
IL-4;
BDNF; or
IL-2, and
an immunomodulatory substance(s), preferably as further active ingredient, wherein in case the pharmaceutical composition (IV) comprises, preferably consists of as active ingredient(s):
IFN-γ, the immunomodulatory substance(s) is different from IFN-γ;
IL-4, the immunomodulatory substance(s) is different from IL-4;
BDNF, the immunomodulatory substance(s) is different from BDNF; and
IL-2, the immunomodulatory substance(s) is different from IL-2.

In an additional general concept, the present invention relates to a dosage form suitable for injection comprising a limited amount of an immunomodulatory substance.

Hence, the present invention relates additionally to an injectable dosage form (II), comprising, preferably consisting of, preferably as active ingredient(s):
an IFN-γ-like acting substance(s) in a total amount equivalent to 30,000 IU IFN-γ or less and/or in a total amount equivalent in activity to 1,500 ng IFN-γ or less;
an IL-4-like acting substance(s) in a total amount equivalent in activity to 1,000 ng IL-4 or less;
an BDNF-like acting substance(s) in a total amount equivalent in activity to 500 ng BDNF or less; or
an IL-2-like acting substance(s) a total amount equivalent to 1,000 IU IL-2 or less and/or in a total amount equivalent in activity to 61 ng IL-2 or less.

Preferably, the IFN-γ-like acting substance is IFN-γ and/or any derivative, fragment, protein-analogue, peptide, protein-variant, biopharmaceutical, inductor, precursor, prodrug, mutein, co-drug, propeptide thereof and/or any pharmaceutically acceptable salt thereof,
the IL-4-like acting substance is IL-4 and/or any derivative, fragment, protein-analogue, peptide, protein-variant, biopharmaceutical, inductor, precursor, prodrug, mutein, co-drug, propeptide thereof and/or any pharmaceutically acceptable salt thereof, the BDNF-like acting substance is BDNF and/or any derivative, fragment, protein-analogue, peptide, protein-variant, biopharmaceutical, inductor, precursor, prodrug, mutein, co-drug, propeptide thereof and/or any pharmaceutically acceptable salt thereof,
the IL-2-like acting substance is IL-2 and/or any derivative, fragment, protein-analogue, peptide, protein-variant, biopharmaceutical, inductor, precursor, prodrug, mutein, co-drug, propeptide thereof and/or any pharmaceutically acceptable salt thereof, still more preferably IFN-γ, IL-4, BDNF and/or IL-2.

Hence, still more preferably, the injectable dosage form (II), comprises, preferably consisting of, preferably as active ingredient(s):
IFN-γ in a total amount of 30,000 IU or less and/or in a total amount of 1,500 ng or less;
IL-4 in a total amount of 1,000 ng or less;
BDNF in a total amount of 500 ng or less; or
IL-2 a total amount of 1,000 IU or less and/or in a total amount of 61 ng or less.

It is to be understood that the total amounts which are comprised in any of the embodiments of the topical dosage forms described herein below are independently and mutatis mutandis applicable to the total amounts which are comprised in the injectable dosage form (II).

In a further general concept, the present invention relates to a dosage form suitable for topical application and administration comprising at least one immunomodulatory substance or comprising any of the pharmaceutical compositions as described herein.

Moreover, the present invention relates to a topical dosage form (I) comprising, preferably consisting of, preferably as active ingredient(s):
an IFN-γ-like acting substance;
an IL-4-like acting substance;
an BDNF-like acting substance; or an IL-2-like acting substance.

Preferably, the IFN-γ-like acting substance is IFN-γ and/or any derivative, fragment, protein-analogue, peptide, protein-variant, biopharmaceutical, inductor, precursor, prodrug, mutein, co-drug, propeptide thereof and/or any pharmaceutically acceptable salt thereof.

Preferably, the IL-4-like acting substance is IL-4 and/or any derivative, fragment, protein-analogue, peptide, protein-variant, biopharmaceutical, inductor, precursor, prodrug, mutein, co-drug, propeptide thereof and/or any pharmaceutically acceptable salt thereof.

Preferably, the BDNF-like acting substance is BDNF and/or any derivative, fragment, protein-analogue, peptide, protein-variant, biopharmaceutical, inductor, precursor, prodrug, mutein, co-drug, propeptide thereof and/or any pharmaceutically acceptable salt thereof.

Preferably, the IL-2-like acting substance is IL-2 and/or any derivative, fragment, protein-analogue, peptide, protein-variant, biopharmaceutical, inductor, precursor, prodrug, mutein, co-drug, propeptide thereof and/or any pharmaceutically acceptable salt thereof.

More preferably, the IFN-γ-like acting substance is IFN-γ, the IL-4-like acting substance is IL-4, the BDNF-like acting substance is BDNF and the IL-2-like acting substance is IL-2.

Hence, more preferably, the topical dosage form (I) comprises, preferably consists of, preferably as active ingredient(s):
IFN-γ;
IL-4;
BDNF; or
IL-2.

Furthermore, the present invention relates to a topical dosage form (II) comprising, preferably consisting of, preferably as active ingredient(s) and/or active component(s):
an immunomodulatory substance(s); and
an energizing constituent.

In a still even further general concept, the present invention relates to a device suitable and/or configured for delivery of at least one immunomodulatory substance and/or suitable and/or configured for performing step (A) of the method as described herein.

Hence, still further, the present invention relates to a medical device (100) comprising:
a skin-conditioning unit; and
a first applicator unit;
wherein the skin-conditioning unit and the first applicator unit are configured to cooperatively interact to administer to a skin of a subject an amount of a first therapeutic agent by means of the first applicator unit with an amount of conditioning measures by means of the skin-conditioning unit; and/or
wherein the skin-conditioning unit and the first applicator unit are configured to act to a skin of a subject.

Preferably, the first therapeutic agent is an immunomodulatory substance(s).

The substance(s), skin-conditioning agent(s), pharmaceutical compositions, dosage forms and/or medical devices as mentioned in any of the embodiments described herein may be independently provided in the form of a kit of parts. Hence, in a further aspect, the present invention relates to a kit of parts comprising an immunomodulatory substance(s). In an even further aspect, the present invention relates to a kit of parts comprising a skin-conditioning agent.

It is to be understood that any embodiment disclosed for the substance(s), skin-conditioning agent(s), pharmaceutical compositions, dosage forms, medical devices, their respective uses thereof and their respective medical uses thereof is independently and mutatis mutandis applicable to the kits of parts.

Hence, the present invention further relates to a kit of parts (I) comprising, preferably consisting of, preferably as active ingredient(s) and/or active component(s):
i) an immunomodulatory substance(s); and
ii) a skin-conditioning agent; and/or
iii) an energizing means.

In a further aspect, the present invention relates to a kit of parts comprising two or more different immunomodulatory substance(s). Therefore, the present invention further relates to a kit of parts (II) comprising, preferably consisting of, preferably as active ingredient(s):
i) an IFN-γ-like acting substance, IL 4-like acting substance, BDNF-like acting substance or IL-2-like acting substance; and
ii) an immunomodulatory substance(s), wherein in case the Kit of parts (II) comprises:
   the IFN-γ-like acting substance, the immunomodulatory substance(s) is different from the IFN-γ-like acting substance;
   the IL-4-like acting substance, the immunomodulatory substance(s) is different from the IL-4-like acting substance;
   the BDNF-like acting substance, the immunomodulatory substance(s) is different from the BDNF-like acting substance; and
   the IL-2-like acting substance, the immunomodulatory substance(s) is different from the IL-2-like acting substance.

Preferably, the IFN-γ-like acting substance is IFN-γ and/or any derivative, fragment, protein-analogue, peptide, protein-variant, biopharmaceutical, inductor, precursor, prodrug, mutein, co-drug, propeptide thereof and/or any pharmaceutically acceptable salt thereof.

Preferably, the IL-4-like acting substance is IL-4 and/or any derivative, fragment, protein-analogue, peptide, protein-variant, biopharmaceutical, inductor, precursor, prodrug, mutein, co-drug, propeptide thereof and/or any pharmaceutically acceptable salt thereof.

Preferably, the BDNF-like acting substance is BDNF and/or any derivative, fragment, protein-analogue, peptide, protein-variant, biopharmaceutical, inductor, precursor, prodrug, mutein, co-drug, propeptide thereof and/or any pharmaceutically acceptable salt thereof.

Preferably, the IL-2-like acting substance is IL-2 and/or any derivative, fragment, protein-analogue, peptide, protein-variant, biopharmaceutical, inductor, precursor, prodrug, mutein, co-drug, propeptide thereof and/or any pharmaceutically acceptable salt thereof.

More preferably, the IFN-γ-like acting substance is IFN-γ, the IL-4-like acting substance is IL-4, the BDNF-like acting substance is BDNF and the IL-2-like acting substance is IL-2.

In an even further aspect, the present invention relates to a kit of parts comprising the injectable dosage forms as mentioned in any of the embodiments described herein.

Particularly, the present invention further relates to a kit of parts (III) comprising:
i) an injectable dosage form as mentioned in any of the embodiments described herein; and
ii) a skin-conditioning agent; and/or
iii) an energizing means.

Preferably, the injectable dosage form is the injectable dosage form (I), (II) and/or (X), more preferably the injectable dosage form (I) and/or (II) as mentioned in any of the embodiments described herein.

In a still further aspect, the present invention relates to a kit of parts comprising the topical dosage forms as mentioned in any of the embodiments described herein.

Particularly, the present invention relates to a kit of parts kit of parts (IV) comprising:
i) a topical dosage form as mentioned in any of the embodiments described herein; and
ii) a skin-conditioning agent; and/or
iii) an energizing means.

Preferably, the topical dosage form is the topical dosage form (I), (II) and/or (X), more preferably the topical dosage form (I) as mentioned in any of the embodiments described herein.

In the kits of parts the substance(s) and/or skin-conditioning agent(s) stated in i) and/or ii) may be provided in the form of any pharmaceutical acceptable formulation like for instance in the form of pharmaceutical compositions, topical dosage forms, medical devices and/or injectable dosage forms. Preferably, in the kit of parts the substance(s) and/or skin-conditioning agent(s) stated in i) and/or ii) are provided in the form of any of the pharmaceutical compositions, the topical dosage forms, the injectable dosage forms, the medical devices or any combination thereof as mentioned in any of the embodiments described herein.

The energizing means stated in iii) of the kits of parts (I), (III), (IV) and/or (V) may be any energizing means as mentioned in any of the embodiments described herein. Preferably, the energizing means is an apparative energizing means as mentioned in any of the embodiments described herein, more preferably a heating means, even more preferably an apparative heating means, as mentioned in any of the embodiments described herein, particularly as mentioned in step (A-6) of the method as described herein. Hence, if not mentioned otherwise, it is to be understood that any embodiment or definition described herein, particularly any embodiment or definition of the conditioning energy, the energizing means and/or the heating means described herein for step (A-6) of the method is independently and mutatis mutandis applicable to the energizing means and/or the heating means as mentioned in respect of the kits of parts (I), (III), (IV) and/or (V).

In a still even further aspect, the present invention further relates to a kit of parts comprising the medical device as mentioned in any of the embodiments described herein.

Particularly, the present invention relates to a kit of parts Kit of parts (V) comprising
an applicator unit (120); and
a skin-conditioning unit (110); and
optionally a reservoir (121), comprising a therapeutic agent like an immunomodulatory substance, wherein the reservoir (121) is configured to be fluidly coupled to the applicator unit (120).

Preferably, the skin-conditioning unit (110) and/or the applicator unit (120) is configured to be mechanically and/or electrically coupled to cooperatively interact to administer to a skin (102) of a subject an amount of a first therapeutic agent by means of the applicator unit (120) with an amount of conditioning measures by means of the skin-conditioning unit (110).

Preferably, the immunomodulatory substance is in the pharmaceutical composition is the injectable dosage form (II), (III), (IV) and/or (X), more preferably the injectable dosage form (II), (III) and/or (IV) as mentioned in any of the embodiments disclosed herein and the injectable dosage form is the injectable dosage form (I), (II) and/or (X), more preferably the injectable dosage form (I) and/or (II) as mentioned in any of the embodiments disclosed herein.

Furthermore, the present invention relates to any pharmaceutical composition, any injectable dosage form, any topical dosage form, any medical device and/or any kit of parts which comprise an immunomodulatory substance(s) and which are configured and/or suitable for use in the method as mentioned in any of the embodiments described herein. Hence, the pharmaceutical composition, any injectable dosage form, any topical dosage form, any medical device and/or any kit of parts which comprise an immunomodulatory substance(s) and which may be configured and/or suitable for use in at least parts of the steps or all steps the method as mentioned in any of the embodiments described herein.

Moreover, the present invention relates to any pharmaceutical composition, any injectable dosage form, any topical dosage form, any medical device and/or any kit of parts which comprise an immunomodulatory substance(s) and which are for use in the method as mentioned in any of the embodiments described herein.

Furthermore, the present invention relates to any pharmaceutical composition, any injectable dosage form, any topical dosage form, any medical device and/or any kit of parts which comprise a skin-conditioning agent and which are configured and/or suitable for use in the method as mentioned in any of the embodiments described herein. Hence, the pharmaceutical composition, any injectable dosage form, any topical dosage form, any medical device and/or any kit of parts which comprise a skin-conditioning agent and which may be configured and/or suitable for use in at least parts of the steps or all steps the method as mentioned in any of the embodiments described herein.

Moreover, the present invention relates to any pharmaceutical composition, any injectable dosage form, any topical dosage form, any medical device and/or any kit of parts which comprise a skin-conditioning agent and which are for use in the method as mentioned in any of the embodiments described herein.

Hence, the present invention further relates to a pharmaceutical composition (X), injectable dosage forms (X), topical dosage form (X), medical device (X) and/or a kit of parts (X) comprising:
a skin-conditioning agent; or
an immunomodulatory substance(s),
suitable, preferably configured, for use in a method for treating and/or preventing an inflammatory disease, immunological disease and/or autoimmunological disease in a subject,
wherein the method comprises, preferably consists of, a step (A), wherein step (A) is selected from one or more of steps:
(A-0) generating an accumulation of PBMCs within the skin of the subject;
(A-1) generating a vasodilation of the capillaries within the skin of the subject;
(A-2) generating an increased blood volume within the skin of the subject;
(A-3) generating an increased sO₂ within the skin and/or an increased rHb within the skin of the subject;
(A-4) generating an increased temperature on the skin of the subject;
(A-5) generating a redness on the skin of the subject;
(A-6) administering conditioning energy to the skin of the subject;
(A-7) administering a skin-conditioning agent to the skin of the subject; and/or
(A-8) administering PBMCs into the skin of the subject,
and the method further comprises the step(s) of;
(B) administering the immunomodulatory substance(s) to the skin of said subject; and/or
(C) administering the immunomodulatory substance(s) to the skin of the subject,
wherein the immunomodulatory substance(s) administered in step (C) is different from the immunomodulatory substance(s) administered in step (B).

Preferably, the pharmaceutical composition (X), injectable dosage forms (X), topical dosage form (X), medical device (X) and/or kit of parts (X) comprising:
a skin-conditioning agent; or
an immunomodulatory substance(s),
is for use in a method for treating and/or preventing an inflammatory disease, immunological disease and/or autoimmunological disease in a subject,
wherein the method comprises, preferably consists of, a step (A), wherein step (A) is selected from one or more of steps:
(A-0) generating an accumulation of PBMCs within the skin of the subject;
(A-1) generating a vasodilation of the capillaries within the skin of the subject;
(A-2) generating an increased blood volume within the skin of the subject;
(A-3) generating an increased sO₂ within the skin and/or an increased rHb within the skin of the subject;
(A-4) generating an increased temperature on the skin of the subject;
(A-5) generating a redness on the skin of the subject;
(A-6) administering conditioning energy to the skin of the subject;
(A-7) administering a skin-conditioning agent to the skin of the subject; and/or
(A-8) administering PBMCs into the skin of the subject,
and the method further comprises the step(s) of;
(B) administering the immunomodulatory substance(s) to the skin of said subject; and/or
(C) administering the immunomodulatory substance(s) to the skin of the subject,
wherein the immunomodulatory substance(s) administered in step (C) is different from the immunomodulatory substance(s) administered in step (B).

In an even further aspect, the present invention relates to the use of any or all of the injectable dosages forms, topical dosage forms, medical devices and/or kit of parts as mentioned in any of the embodiments described herein in the method for treating and/or preventing an inflammatory disease, immunological disease and/or autoimmunological disease in a subject.

In a still even further aspect, the present invention relates to the use of any or all of the pharmaceutical compositions as mentioned in any of the embodiments described herein for producing an article or a medical device.

Moreover, the present invention relates to the use of any or all of the injectable dosage forms as mentioned in any of the embodiments described herein for producing a medical device.

If not mentioned otherwise, it is to be understood that any of the embodiments mentioned under the following outline notes:
‘As further regards independently any or all of the pharmaceutical compositions, particularly the pharmaceutical compositions (I), (II), (III), (IV) and/or CO, for any of the embodiments as described herein’; ‘As further regards independently any or all of the injectable dosage forms, particularly the injectable dosage form (I), (II) and/or (X), for any of the embodiments as described herein’;
‘As further regards independently any or all of the topical dosage forms, particularly the topical dosage form (I), (II) and/or (X), for any of the embodiments as described herein’;
‘As further regards the medical device (100) for any of the embodiments as described herein’; and
‘As further regards independently any or all of the kits of parts, particularly the kits of parts (I), (II), (III), (IV), (V) and/or CO, for any of the embodiments as described herein’,
are independently applicable to each other and/or combinable with each other and with any of the embodiments as described herein. The outline notes merely serve to structure the text and are not intended to have any delimiting or restrictive meaning beyond that. Generally, if not mentioned otherwise, it is to be understood that any embodiment or definition described herein in terms of the pharmaceutical compositions, injectable dosage forms, topical dosage forms, medical devices, kits of parts, respective uses thereof and/or medical uses thereof is independently and mutatis mutandis applicable to the method and/or medical use as mentioned in any of the embodiments described herein, and vice versa.

As further regards independently any or all of the pharmaceutical compositions, the injectable dosage forms, the topical dosage forms, the medical devices and/or the kits of parts for any of the embodiments as described herein

Preferably, where applicable, in the pharmaceutical compositions, the injectable dosage forms, the topical dosage forms, the medical devices and/or the kits of parts the immunomodulatory substance(s) is a cytokine-like acting substance(s), preferably an interferon-like acting substance(s), interleukin-like acting substance(s) and/or neurotrophin-like acting substance(s), more preferably an IFN-γ-like acting substance(s), IL-4-like acting substance(s), BDNF-like acting substance(s) and/or IL-2 like acting substance(s), even more preferably IFN-γ, IL-4, BDNF, IL-2 and/or any derivative, fragment, protein-analogue, peptide, protein-variant, biopharmaceutical, inductor, precursor, prodrug, mutein, co-drug, propeptide thereof and/or any pharmaceutically acceptable salt of any of these, still even more preferably:
IFN-γ;
IL-4;
BDNF; and/or
IL-2.

Further preferably,
in the pharmaceutical compositions, the injectable dosage forms, the topical dosage forms, the medical devices and/or the kits of parts the immunomodulatory substance(s) is; and/or
the pharmaceutical compositions, the injectable dosage forms, the topical dosage forms, the medical devices and/or the kits of parts comprise,
any combination of two, three or four selected from:
   IFN-γ;
   IL-4;
   BDNF; and
   IL-2,
and/or or any derivative, fragment, protein-analogue, peptide, protein-variant, biopharmaceutical, inductor, precursor, prodrug, mutein, co-drug, propeptide thereof and/or any pharmaceutically acceptable salt of any of these.

Even further preferably,
in the pharmaceutical compositions, the injectable dosage forms, the topical dosage forms, the medical devices and/or the kits of parts the immunomodulatory substance(s) is; and/or
the pharmaceutical compositions, the injectable dosage forms, the topical dosage forms, the medical devices and/or the kits of parts comprise,
   IFN-γ and/or IL-4 and IL-2; or
   IL-4 and/or BDNF and IL-2,
and/or or any derivative, fragment, protein-analogue, peptide, protein-variant, biopharmaceutical, inductor, precursor, prodrug, mutein, co-drug, propeptide thereof and/or any pharmaceutically acceptable salt of any of these.

Still further preferably,
in the pharmaceutical compositions, the injectable dosage forms, the topical dosage forms, the medical devices and/or the kits of parts the immunomodulatory substance(s) is; and/or
the pharmaceutical compositions, the injectable dosage forms, the topical dosage forms, the medical devices and/or the kits of parts comprise:
   IFN-γ and IL-2;
   IL-4 and IL-2; or
   BDNF and IL-2,
and/or or any derivative, fragment, protein-analogue, peptide, protein-variant, biopharmaceutical, inductor, precursor, prodrug, mutein, co-drug, propeptide thereof and/or any pharmaceutically acceptable salt of any of these.

The immunomodulatory substance(s), cytokine-like acting substance-like acting substance(s), interferon-like acting substance(s), interleukin-like acting substance(s), neurotrophin-like acting substance(s), IFN-γ-like acting substance(s), IL-4-like acting substance(s), BDNF-like acting substance(s) and IL-2-like acting substance(s), cytokine(s), interferon(s), interleukin(s), neurotrophin(s), IFN-γ, IL-4, BDNF, IL-2, derivative, fragment, protein-analogue, peptide, protein-variant, biopharmaceutical, inductor, precursor, prodrug, mutein, co-drug, propeptide and/or pharmaceutically acceptable salt of any of these of the pharmaceutical compositions, the injectable dosage forms, the topical dosage forms, the medical devices and/or the kit of parts are independently any immunomodulatory substance(s), cytokine-like acting substance-like acting substance(s), interferon-like acting substance(s), interleukin-like acting substance(s), neurotrophin-like acting substance(s), IFN-γ-like acting substance(s), IL-4-like acting substance(s), BDNF-like acting substance(s) and IL-2-like acting substance(s), cytokine(s), interferon(s), interleukin(s), neurotrophin(s), IFN-γ, IL-4, BDNF, IL-2, derivative, fragment, biopharmaceutical, inductor, precursor, prodrug, mutein, co-drug and/or pharmaceutically acceptable salt of any of these as mentioned in any of the embodiments described herein, particularly as mentioned as mentioned in any of the embodiments of the method described herein.

If not mentioned otherwise, it is to be understood that any description and definition of the immunomodulatory substance(s), cytokine-like acting substance-like acting substance(s), interferon-like acting substance(s), interleukin-like acting substance(s), neurotrophin-like acting substance(s), IFN-γ-like acting substance(s), IL-4-like acting substance(s), BDNF-like acting substance(s) and IL-2-like acting substance(s), cytokine(s), interferon(s), interleukin(s), neurotrophin(s), IFN-γ, IL-4, BDNF, IL-2, derivative, fragment, protein-analogue, peptide, protein-variant, biopharmaceutical, inductor, precursor, prodrug, mutein, co-drug, propeptide and/or pharmaceutically acceptable salt of any of these as mentioned in any of the embodiments of the method, particularly in step (B), (B₁) and/or (C) of the method as described herein is independently and mutatis mutandis applicable to the immunomodulatory substance(s), cytokine-like acting substance-like acting substance(s), interferon-like acting substance(s), interleukin-like acting substance(s), neurotrophin-like acting substance(s), IFN-γ-like acting substance(s), IL-4-like acting substance(s), BDNF-like acting substance(s) and IL-2-like acting substance(s), cytokine(s), interferon(s), interleukin(s), neurotrophin(s), IFN-γ, IL-4, BDNF, IL-2, derivative, fragment, protein-analogue, peptide, protein-variant, biopharmaceutical, inductor, precursor, prodrug, mutein, co-drug, propeptide and/or any pharmaceutically acceptable salt of any of these as mentioned in any of the embodiments of the pharmaceutical compositions, the injectable dosage forms, the topical dosage forms, the medical devices and/or the kits of parts described herein.

The skin-conditioning agent(s) of the pharmaceutical compositions, the injectable dosage forms, the topical dosage forms, the medical devices and/or the kit of parts is independently any skin-conditioning agent as mentioned in any of the embodiments described herein. If not mentioned otherwise, it is to be understood that any description and definition of the skin-conditioning agent(s) as mentioned in any of the embodiments of the method, particularly in step (A-7) of the method as described herein is independently and mutatis mutandis applicable to the skin-conditioning agent(s) as mentioned in any of the embodiments of the pharmaceutical compositions, the injectable dosage forms, the topical dosage forms, the medical devices and/or the kits of parts described herein.

In any of the embodiments described herein and particularly the pharmaceutical compositions of the injectable dosage forms, the topical dosage forms, the medical devices and/or the kits of parts, where applicable, the skin-conditioning agent may be any blood-circulation-increasing agent, vasodilating agent, skin-temperature increasing agent, skin-502-increasing agent and/or skin-rHb-increasing agent.

Preferably, in the injectable dosage forms, the topical dosage forms, the medical devices and/or the kits of parts the immunomodulatory substance(s), IFN-γ, IL-4, BDNF and/or IL-2 are independently in the form of a pharmaceutical composition, more preferably in the form of any of the pharmaceutical compositions (I), (II), (III) and/or (IV) as mentioned in any of the embodiments described herein.

Preferably, the injectable dosage forms, the topical dosage forms, the medical devices and/or the kits of parts comprise a pharmaceutical composition which pharmaceutical composition comprises, preferably consists of, the immunomodulatory substance(s), IFN-γ, IL-4, BDNF and/or IL-2, preferably as active ingredient(s). More preferably, the pharmaceutical composition comprised in the injectable dosage forms, the topical dosage forms, the medical devices and/or the kits of parts is any of the pharmaceutical compositions (I), (II), (III) and/or (IV) as mentioned in any of the embodiments described herein.

Preferably, the pharmaceutical compositions of the injectable dosage forms, the topical dosage forms, the medical devices and/or the kits of parts comprise, preferably consist of, the immunomodulatory substance(s), IFN-γ, IL-4, BDNF and/or IL-2 as further active ingredient(s). More preferably, the pharmaceutical compositions of the injectable dosage forms, the topical dosage forms, the medical devices and/or the kits of parts is any of the pharmaceutical compositions (I), (II), (III) and/or (IV) as mentioned in any of the embodiments described herein.

The individual substance(s) and/or skin-conditioning agent(s) can be present within the pharmaceutical compositions, the injectable dosage forms, the topical dosage forms, the medical devices and/or the kits of parts in any of the embodiments described herein in varying amounts. This depends inter alia on the kind and form of the formulation or preparation, the form and route of the intended administration or application and/or on the subject or the disease to be treated and/or prevented. Hence, the effective amount of the comprised substance(s) and/or skin-conditioning agent(s) may vary.

If not mentioned otherwise, it is to be understood that the expression “substance(s)” is used in any of the embodiments of the pharmaceutical compositions, the injectable dosage forms, the topical dosage forms, the medical devices and/or the kits of parts described herein as generic term for the immunomodulatory substance(s); cytokine-like acting substance(s), interferon-like acting substance(s), interleukin-like acting substance(s), neurotrophin-like acting substance(s), IFN-γ-like acting substance(s), IL-2-like acting substance(s), IL-4-like acting substance(s), BDNF-like acting substance(s); cytokine(s), interferon(s), interleukin(s), neurotrophin(s), IFN-γ, IL-4, BDNF and/or IL-2 and/or any derivative, fragment, protein-analogue, peptide, protein-variant, biopharmaceutical, inductor, precursor, prodrug, mutein, co-drug, propeptide thereof and/or any pharmaceutically acceptable salt of any of these; particularly as generic term of the immunomodulatory substance(s), IFN-γ-like acting substance(s), IL-2-like acting substance(s), IL-4-like acting substance(s), BDNF-like acting substance(s), IFN-γ, IL-4, BDNF and/or the IL-2 and/or any derivative, fragment, protein-analogue, peptide, protein-variant, biopharmaceutical, inductor, precursor, prodrug, mutein, co-drug, propeptide thereof and/or any pharmaceutically acceptable salt of any of these; more particular as generic term for the IFN-γ, IL-4, BDNF and/or the IL-2.

Preferably, the pharmaceutical compositions, the injectable dosage forms, the topical dosage forms, the medical devices and/or the kits of parts comprise an effective amount of the substance(s) and/or skin-conditioning agent(s). Particularly, the pharmaceutical compositions, the injectable dosage forms, the topical dosage forms, the medical devices and/or the kits of parts comprise an effective amount of the substance(s).

Preferably, the pharmaceutical compositions, the injectable dosage forms, the topical dosage forms, the medical devices and/or the kits of parts are suitable and/or configured to deliver an effective amount of the substance(s) and/or skin-conditioning agent(s). Particularly, the pharmaceutical compositions, the injectable dosage forms, the topical dosage forms, the medical devices and/or the kits of parts are suitable and/or configured to deliver an effective amount of the substance(s).

If not mentioned otherwise, it is to be understood that any description and definition of the amount or effective amount of the substance(s) mentioned in any of the embodiments of the method, particularly in step (B), (B₁) and/or (C) of the method as described herein is independently and mutatis mutandis applicable to the amount or effective amount of the substance(s) as mentioned in any of the embodiments of the pharmaceutical compositions, the injectable dosage forms, the topical dosage forms, the medical devices and/or the kits of parts described herein.

Preferably, the pharmaceutical compositions, the injectable dosage forms, the topical dosage forms, the medical devices and/or the kits of parts:
are suitable and/or configured not to cause a systemic increase, preferably an effective system increase, of the concentration of the substance(s) in the subject; and/or
they comprise the substance(s) in an amount that does not cause a systemic increase, preferably an effective system increase, of the concentration of the substance(s) in the subject; and/or
they are suitable and/or configured to administer, apply and/or deliver the substance(s) in an amount that does not cause a systemic increase, preferably an effective system increase, of the concentration of the substance(s) in the subject.

Preferably, the pharmaceutical compositions, the injectable dosage forms, the topical dosage forms, the medical devices and/or the kits of parts:
are suitable and/or configured not to cause a systemic activation, preferably an effective systemic activation, of the respective receptor of the substance(s); and/or
they comprise the substance(s) in an amount that does not cause a systemic activation, preferably an effective systemic activation, of the respective receptor of the substance(s); and/or
they are suitable and/or configured to administer, apply and/or deliver the substance(s) in an amount that does not cause a systemic activation, preferably an effective systemic activation, of the respective receptor of the substance(s).

The receptor of the substance(s) is for instance a respective receptor of the immunomodulatory substance(s), a respective receptor of the cytokine(s), a respective receptor of the interferon(s), a respective receptor of the interleukin(s), a respective receptor of the neurotrophin(s), a respective receptor of the IFN-γ, a respective receptor of the IL-4, a respective receptor of the BDNF and/or a respective receptor of the IL-2.

Preferably, the pharmaceutical compositions, the injectable dosage forms, the topical dosage forms, the medical devices and/or the kits of parts:
are suitable and/or configured not to cause a systemic generation, preferably an effective systemic generation, of the substance(s) in the subject and/or the generated increased concentration, preferably the generated effective increased concentration of the substance(s) administered is only local but not systemic in the subject; and/or
they comprise the substance(s) in an amount that does not cause a systemic generation, preferably an effective systemic generation, of the substance(s) in the subject and/or the generated increased concentration, preferably the generated effective increased concentration of the substance(s) administered is only local but not systemic in the subject; and/or
they are suitable and/or configured to administer, apply and/or deliver the substance(s) in an amount that does not cause a systemic generation, preferably an effective systemic generation, of the substance(s) in the subject and/or the generated increased concentration, preferably the generated effective increased concentration of the substance(s) administered is only local but not systemic in the subject.

More preferably, where applicable, the pharmaceutical compositions, the injectable dosage forms, the topical dosage forms, the medical devices and/or the kits of parts are and particularly the pharmaceutical composition (II) is suitable and/or configured to deliver, preferably to the skin of the subject, IFN-γ-like acting substance(s), IL-4-like acting substance(s), BDNF-like acting substance(s) and/or IL-2-like acting substance(s) in the following amount:
IFN-γ-like acting substance(s)
in an amount equivalent to 600 IU IFN-γ/kg body mass of the subject or less;
in an amount equivalent in activity to 30 ng IFN-γ/kg body mass of the subject or less;
in a total amount equivalent to 30,000 IU IFN-γ or less; and/or
in a total amount equivalent in activity to 1,500 ng IFN-γ or less;
IL-4-like acting substance(s)
in an amount equivalent in activity to 20 ng IL-4/kg body mass of the subject or less; and/or
in a total amount equivalent in activity to 1,000 ng IL-4 or less;
BDNF-like acting substance(s)
in an amount equivalent in activity to 10 ng BDNF/kg per body mass of the subject or less; and/or
in a total amount equivalent in activity to 500 ng BDNF or less; and/or
IL-2-like acting substance(s) in an amount equivalent to 10 IU/kg body mass of the subject or less;
in an amount of 0.6 ng/kg body mass of the subject or less;
in a total amount equivalent in activity to 500 IU IL-2 or less; and/or
in a total amount equivalent in activity to 30.5 ng IL-2 or less.

Preferably, the amount and/or total amounts of the IFN-γ-like acting which the pharmaceutical compositions, the injectable dosage forms, the topical dosage forms, the medical devices and/or the kits of parts are suitable and/or configured to deliver, are as follows;
IFN-γ-like acting in an amount, preferably a total amount, equivalent to 600 IU IFN-γ/kg (international unit(s)/kilogram) body mass of the subject or less (‘kg body mass’ is in its meaning equivalent to ‘kg of the body mass’), preferably per administration dose, to the skin of the subject.
More preferably, IFN-γ-like acting in an amount, preferably a total amount, equivalent to 300 IU IFN-γ/kg or less, even more preferably 200 IU IFN-γ/kg or less, still more preferably 100 IU IFN-γ/kg or less, still even more preferably 50 IU IFN-γ/kg or less, further preferably 30 IU IFN-γ/kg or less, further preferably 20 IU IFN-γ/kg or less, even further preferably 6 IU IFN-γ/kg or less.
There is no lower limit for the amount per kg body mass as long as the beneficial effects can be achieved, however, usually, a lower limit is an amount equivalent to 0.04 IU IFN-γ/kg or more, more preferably 0.2 IU IFN-γ/kg or more, even more preferably, 0.5 IU IFN-γ/kg or more, still more preferably 1 IU IFN-γ/kg or more, still even more preferably 1.5 IU IFN-γ/kg or more.
More preferably, IFN-γ-like acting in an amount, preferably a total amount, in the range of equivalent to 0.04 IU IFN-γ/kg or more and 600 IU IFN-γ/kg or less, even more preferably 0.2 IU IFN-γ/kg or more and 300 IU IFN-γ/kg or less, still more preferably 0.5 IU IFN-γ/kg or more and 200 IU IFN-γ/kg or less, still even more preferably 0.5 IU IFN-γ/kg or more and 100 IU IFN-γ/kg or less, further preferably 0.5 IU IFN-γ/kg or more and 50 IU IFN-γ/kg or less, even further preferably 1 IU IFN-γ/kg or more and IU IFN-γ/kg or less, still further preferably 1.5 IU IFN-γ/kg or more and 20 IU IFN-γ/kg or less.
Preferably, IFN-γ-like acting in an amount, preferably a total amount, equivalent in activity to 30 ng IFN-γ/kg body mass of the subject or less, preferably per administration dose, to the skin of the subject. More preferably, IFN-γ-like acting in an amount, preferably a total amount, equivalent in activity to 15 ng IFN-γ/kg or less, even more preferably 10 ng IFN-γ/kg or less, still more preferably 5 ng IFN-γ/kg or less, still even more preferably 2.5 ng IFN-γ/kg or less, further preferably 1.5 ng IFN-γ/kg or less, still even more preferably 1 ng IFN-γ/kg or less, even further preferably 0.3 ng IFN-γ/kg or less.
There is no lower limit for the amount per kg body mass as long as the beneficial effects can be achieved, however, usually, a lower limit for the amount is equivalent in activity to 0.002 ng IFN-γ/kg or more, more preferably 0.025 ng IFN-γ/kg or more, even more preferably 0.01 ng IFN-γ/kg or more, still more preferably 0.02 mg/kg or more, still even more preferably 0.05 ng IFN-γ/kg or more, further preferably 0.075 ng IFN-γ/kg or more.
More preferably, IFN-γ-like acting substance(s), in an amount, preferably a total amount, in the range of equivalent in activity to ng IFN-γ/kg or less and 0.002 ng IFN-γ/kg or more, even more preferably 15 ng IFN-γ/kg or less and 0.025 ng IFN-γ/kg or more, still more preferably 10 ng IFN-γ/kg or less and 0.01 ng IFN-γ/kg or more, still even more preferably 5 ng IFN-γ/kg or less and 0.01 ng IFN-γ/kg or more, further preferably 2.5 ng IFN-γ/kg or less and 0.02 ng IFN-γ/kg or more, even further preferably 1.5 ng IFN-γ/kg or less and 0.02 ng IFN-γ/kg or more, still further preferably 1 ng IFN-γ/kg or less and 0.05 ng IFN-γ/kg or more, still even further preferably 0.3 ng IFN-γ/kg or less and 0.075 ng IFN-γ/kg or more.
The same amounts apply to any derivative, fragment, protein-analogue, peptide, protein-variant, biopharmaceutical, inductor, precursor, prodrug, mutein, co-drug, propeptide and/or any pharmaceutically acceptable salt of the IFN-γ.

Preferably, the amount and/or total amounts of IL-2-like acting substance which the pharmaceutical compositions, the injectable dosage forms, the topical dosage forms, the medical devices and/or the kits of parts are suitable and/or configured to deliver, are as follows, namely:
IL-2-like acting substance in an amount, preferably a total amount, equivalent to 20 IU IL-2/kg body mass of a subject or less, more preferably 10 IU IL-2/kg body mass of a subject or less, preferably per administration dose, to a skin of the subject. Even more preferably, preferably IL-2-like acting substance(s), in an amount equivalent to 8 IU IL-2/kg or less, still more preferably 6 IU IL-2/kg, still even more preferably 5 IU IL-2/kg or less, further preferably 4 IU IL-2/kg or less.
There is no lower limit for the amount, preferably the total amount, per kg body mass as long as the beneficial effects can be achieved, however, usually, a lower limit is an amount equivalent to 0.5 IU IL-2/kg or more, more preferably 1 IU IL-2/kg or more, even more preferably 2 IU IL-2/kg or more.
More preferably, IL-2-like acting substance in an amount, preferably a total amount, in the range of equivalent to 20 IU IL-2/kg or less and 0.5 IU IL-2/kg or more, even more preferably 10 IU IL-2/kg or less and 0.5 IU IL-2/kg or more, still more preferably 8 IU IL-2/kg or less and 0.5 IU IL-2/kg or more, still even more preferably 6 IU IL-2/kg or less and 1 IU IL-2/kg or more, further preferably 5 IU IL-2/kg or less and 1 IU IL-2/kg or more, even further preferably 4 IU IL-2/kg or less and 1 IU IL-2/kg or more.
IL-2-like acting substance in an amount, preferably a total amount, equivalent in activity to 1.2 ng IL-2/kg body mass of a subject or less, more preferably 0.6 ng IL-2/kg body mass of a subject or less, preferably per administration dose, to a skin of the subject. Even more preferably, IL-2, preferably IL-2-like acting substance(s), in an amount equivalent in activity to 0.5 ng IL-2/kg or less, still more preferably 0.37 ng IL-2/kg, still even more preferably 0.3 ng IL-2/kg or less, further preferably 0.25 ng IL-2/kg or less.
There is no lower limit for the amount, preferably the total amount, per kg body mass as long as the beneficial effects can be achieved, however, usually, a lower limit is an amount equivalent in activity to 0.03 ng IL-2/kg or more, more preferably 0.06 ng IL-2/kg or more, even more preferably 0.12 ng IL-2/kg or more.
More preferably, IL-2-like acting substance in an amount, preferably a total amount, in the range of equivalent in activity to 1.2 ng IL-2/kg or less and 0.03 ng IL-2/kg or more, even more preferably 0.6 ng IL-2/kg or less and 0.03 ng IL-2/kg or more, still more preferably 0.5 ng IL-2/kg or less and 0.03 ng IL-2/kg or more, still even more preferably 0.37 ng IL-2/kg or less and 0.06 ng IL-2/kg or more, further preferably 0.3 ng IL-2/kg or less and 0.06 ng IL-2/kg or more, even further preferably 0.25 ng IL-2/kg or less and 0.12 ng IL-2/kg or more.
The same amounts apply to any derivative, fragment, protein-analogue, peptide, protein-variant, biopharmaceutical, inductor, precursor, prodrug, mutein, co-drug, propeptide and/or any pharmaceutically acceptable salt of the IL-2.

Preferably, the amount and/or total amounts of IL-4-like acting substance which the pharmaceutical compositions, the injectable dosage forms, the topical dosage forms, the medical devices and/or the kits of parts are suitable and/or configured to deliver, are as follows, namely:
IL-4-like acting substance in an amount, preferably a total amount, equivalent in activity to 20 ng IL-4/kg body mass of the subject or less, preferably per administration dose, to the skin of the subject. More preferably, IL-4-like acting substance in an amount, preferably a total amount, equivalent in activity to 10 ng IL-4/kg or less, even more preferably 5 ng IL-4/kg or less, still more preferably 2.5 ng IL-4/kg or less, still even more preferably 1.5 ng IL-4/kg or less, further preferably 1 ng IL-4/kg or less, still even more preferably 0.4 ng IL-4/kg or less, even further preferably 0.2 ng IL-4/kg or less.
There is no lower limit for the amount, preferably the total amount, per kg body mass as long as the beneficial effects can be achieved, however, usually, a lower limit for the amount is equivalent in activity to 0.002 ng IL-4/kg or more, more preferably 0.025 ng IL-4/kg or more, even more preferably 0.01 ng IL-4/kg or more, still more preferably 0.02 mg/kg or more, still even more preferably 0.05 ng IL-4/kg or more, further preferably 0.075 ng IL-4/kg or more.
More preferably, IL-4-like acting substance in an amount, preferably a total amount, in the range of equivalent in activity to ng IL-4/kg or less and 0.002 ng IL-4/kg or more, even more preferably 10 ng IL-4/kg or less and 0.025 ng IL-4/kg or more, still more preferably 5 ng IL-4/kg or less and 0.01 ng IL-4/kg or more, still even more preferably 2.5 ng IL-4/kg or less and 0.01 ng IL-4/kg or more, further preferably 1.5 ng IL-4/kg or less and 0.02 ng IL-4/kg or more, even further preferably 1 ng IL-4/kg or less and 0.02 ng IL-4/kg or more, still further preferably 0.4 ng IL-4/kg or less and 0.05 ng IL-4/kg or more and still even further preferably 0.2 ng IL-4/kg or less and 0.075 ng IL-4/kg or more.
The same amounts apply to any derivative, fragment, protein-analogue, peptide, protein-variant, biopharmaceutical, inductor, precursor, prodrug, mutein, co-drug, propeptide and/or pharmaceutically acceptable salt of the IL-4.

Preferably, the amount and/or total amounts of the BDNF-like acting substance which the pharmaceutical compositions, the injectable dosage forms, the topical dosage forms, the medical devices and/or the kits of parts are suitable and/or configured to deliver, are as follows, namely:
BDNF-like acting substance in an amount, preferably a total amount, equivalent in activity to 10 ng BDNF/kg body mass of the subject or less, preferably per administration dose, to the skin of the subject. More preferably, BDNF in an amount of 5 ng BDNF/kg or less, even more preferably 2.5 ng BDNF/kg or less, still more preferably 1 ng BDNF/kg or less, still even more preferably 0.5 ng BDNF/kg or less, further preferably 0.3 ng BDNF/kg or less, still even more preferably 0.1 ng BDNF/kg or less, further preferably 0.05 ng BDNF/kg or less.
There is no lower limit for the amount, preferably the total amount, per kg body mass as long as the beneficial effects can be achieved, however, usually, a lower limit for the amount is equivalent in activity to 0.001 ng BDNF/kg or more, more preferably 0.005 ng BDNF/kg or more, even more preferably 0.007 ng BDNF/kg or more, still more preferably 0.01 mg/kg or more.
More preferably, BDNF-like acting substance in an amount, preferably a total amount, in the range of equivalent in activity to ng BDNF/kg or less and 0.001 ng BDNF/kg or more, even more preferably 5 ng BDNF/kg or less and 0.001 ng BDNF/kg or more, still more preferably 2.5 ng BDNF/kg or less and 0.005 ng BDNF/kg or more, still even more preferably 1 ng BDNF/kg or less and 0.005 ng BDNF/kg or more, further preferably 0.5 ng BDNF/kg or less and 0.007 ng BDNF/kg or more, even further preferably 0.3 ng BDNF/kg or less and 0.007 ng BDNF/kg or more, still further preferably 0.1 ng BDNF/kg or less and 0.01 ng BDNF/kg or more, still even further preferably 0.05 ng BDNF/kg or less and 0.01 ng BDNF/kg or more.
The same amounts apply to any derivative, fragment, biopharmaceutical, inductor, precursor, prodrug, mutein, co-drug and/or pharmaceutically acceptable salt of the BDNF.

Preferably, the amount of the IFN-γ-like acting substance(s), IL-4-like acting substance(s), BDNF-like acting substance(s) and/or IL-2-like acting substance(s) to be delivered is the total amount.

Preferably, the amount of the IFN-γ-like acting substance(s), IL-4-like acting substance(s), BDNF-like acting substance(s) and/or IL-2-like acting substance(s) is the amount delivered per administration dose. More preferably, the total amount of the IFN-γ-like acting substance(s), IL-4-like acting substance(s), BDNF-like acting substance(s) and/or IL-2-like acting substance(s) is the total amount delivered per administration dose.

Preferably, the IFN-γ-like acting substance is IFN-γ and/or any derivative, fragment, protein-analogue, peptide, protein-variant, biopharmaceutical, inductor, precursor, prodrug, mutein, co-drug, propeptide thereof and/or pharmaceutically acceptable salt of any of these,
the IL-4-like acting substance is IL-4 and/or any derivative, fragment, protein-analogue, peptide, protein-variant, biopharmaceutical, inductor, precursor, prodrug, mutein, co-drug, propeptide thereof and/or pharmaceutically acceptable salt of any of these,
the BDNF-like acting substance is BDNF and/or any derivative, fragment, protein-analogue, peptide, protein-variant, biopharmaceutical, inductor, precursor, prodrug, mutein, co-drug, propeptide thereof and/or any pharmaceutically acceptable salt of any of these,
the IL-2-like acting substance is IL-2 and/or any derivative, fragment, protein-analogue, peptide, protein-variant, biopharmaceutical, inductor, precursor, prodrug, mutein, co-drug, propeptide thereof and/or any pharmaceutically acceptable salt of any of these, still more preferably IFN-γ, IL-4, BDNF and/or IL-2.

Hence, even more preferably, the pharmaceutical compositions, the injectable dosage forms, the topical dosage forms, the medical devices and/or the kits of parts are and particularly the pharmaceutical composition (II) is suitable and/or configured to deliver, preferably to the skin of the subject, IFN-γ, IL-4, BDNF, and/or IL-2 in the following amount(s):
IFN-γ in an amount of 600 IU/kg body mass of the subject or less, in an amount of 30 ng/kg body mass of the subject or less, in a total amount of 30,000 IU or less and/or in a total amount of 1,500 ng or less;
IL-4 in an amount of 20 ng/kg body mass of the subject or less and/or in a total amount of 1,000 ng or less;
BDNF in an amount of 10 ng/kg per body mass of the subject or less and/or in a total amount of 500 ng or less; and/or
IL-2 in an amount of 20 IU/kg body mass of the subject or less, in an amount of 1.2 ng/kg body mass of the subject or less, in a total amount of 1,000 IU or less and/or in a total amount of 61 ng or less.

Preferably, the amount and/or total amounts of the IFN-γ-like acting and/or the IFN-γ which the pharmaceutical compositions, the injectable dosage forms, the topical dosage forms, the medical devices and/or the kits of parts are suitable and/or configured to deliver, are as follows.
IFN-γ in an amount, preferably a total amount, of 600 IU/kg (international unit(s)/kilogram) body mass of the subject or less (‘kg body mass’ is in its meaning equivalent to ‘kg of the body mass’), preferably per administration dose, to the skin of the subject.
More preferably, IFN-γ in an amount, preferably a total amount, of 300 IU/kg or less, even more preferably 200 IU/kg or less, still more preferably 100 IU/kg or less, still even more preferably 50 IU/kg or less, further preferably 30 IU/kg or less, further preferably IU/kg or less, even further preferably 6 IU/kg or less.
There is no lower limit for the amount per kg body mass as long as the beneficial effects can be achieved, however, usually, a lower limit is 0.04 IU/kg or more, more preferably 0.2 IU/kg or more, even more preferably, 0.5 IU/kg or more, still more preferably 1 IU/kg or more, still even more preferably 1.5 IU/kg or more.
More preferably, IFN-γ in an amount, preferably a total amount, in the range of 0.04 IU/kg or more and 600 IU/kg or less, even more preferably 0.2 IU/kg or more and 300 IU/kg or less, still more preferably 0.5 IU/kg or more and 200 IU/kg or less, still even more preferably 0.5 IU/kg or more and 100 IU/kg or less, further preferably 0.5 IU/kg or more and 50 IU/kg or less, even further preferably 1 IU/kg or more and 30 IU/kg or less, still further preferably 1.5 IU/kg or more and 20 IU/kg or less.
Preferably, IFN-γ in an amount, preferably a total amount, of 30 ng/kg body mass of the subject or less, preferably per administration dose, to the skin of the subject. More preferably, IFN-γ in an amount, preferably a total amount, of 15 ng/kg or less, even more preferably 10 ng/kg or less, still more preferably 5 ng/kg or less, still even more preferably 2.5 ng/kg or less, further preferably 1.5 ng/kg or less, still even more preferably 1 ng/kg or less, even further preferably 0.3 ng/kg or less. There is no lower limit for the amount per kg body mass as long as the beneficial effects can be achieved, however, usually, a lower limit is 0.002 ng/kg or more, more preferably 0.025 ng/kg or more, even more preferably 0.01 ng/kg or more, still more preferably 0.02 mg/kg or more, still even more preferably 0.05 ng/kg or more, further preferably 0.075 ng/kg or more. More preferably, IFN-γ in an amount, preferably a total amount, in the range of 30 ng/kg or less and 0.002 ng/kg or more, even more preferably 15 ng/kg or less and 0.025 ng/kg or more, still more preferably 10 ng/kg or less and 0.01 ng/kg or more, still even more preferably 5 ng/kg or less and 0.01 ng/kg or more, further preferably 2.5 ng/kg or less and 0.02 ng/kg or more, even further preferably 1.5 ng/kg or less and 0.02 ng/kg or more, still further preferably 1 ng/kg or less and 0.05 ng/kg or more, still even further preferably 0.3 ng/kg or less and 0.075 ng/kg or more.

Preferably, the amount and/or total amounts of the IL-2 which the pharmaceutical compositions, the injectable dosage forms, the topical dosage forms, the medical devices and/or the kits of parts are suitable and/or configured to deliver, are as follows, namely:
IL-2 in an amount, preferably a total amount, of 20 IU/kg body mass of a subject or less, more preferably 10 IU/kg body mass of a subject or less, preferably per administration dose, to a skin of the subject. Even more preferably, IL-2 in an amount of 8 IU/kg or less, still more preferably 6 IU/kg, still even more preferably 5 IU/kg or less, further preferably 4 IU/kg or less.
There is no lower limit for the amount, preferably the total amount, per kg body mass as long as the beneficial effects can be achieved, however, usually, a lower limit is 0.5 IU/kg or more, more preferably 1 IU/kg or more, even more preferably 2 IU/kg or more.
More preferably, IL-2 in an amount, preferably a total amount, in the range of 20 IU/kg or less and 0.5 IU/kg or more, even more preferably 10 IU/kg or less and 0.5 IU/kg or more, still more preferably 8 IU/kg or less and 0.5 IU/kg or more, still even more preferably 6 IU/kg or less and 1 IU/kg or more, further preferably 5 IU/kg or less and 1 IU/kg or more, even further preferably 4 IU/kg or less and 1 IU/kg or more.
IL-2 in an amount, preferably a total amount, of 1.2 ng/kg body mass of a subject or less, more preferably 0.6 ng/kg body mass of a subject or less, preferably per administration dose, to a skin of the subject. Even more preferably, IL-2 in an amount of 0.5 ng/kg or less, still more preferably 0.37 ng/kg, still even more preferably 0.3 ng/kg or less, further preferably 0.25 ng/kg or less.
There is no lower limit for the amount, preferably the total amount, per kg body mass as long as the beneficial effects can be achieved, however, usually, a lower limit is 0.03 ng/kg or more, more preferably 0.06 ng/kg or more, even more preferably 0.12 ng/kg or more. More preferably, IL-2 in an amount, preferably a total amount, in the range of 1.2 ng/kg or less and 0.03 ng/kg or more, even more preferably 0.6 ng/kg or less and 0.03 ng/kg or more, still more preferably 0.5 ng/kg or less and 0.03 ng/kg or more, still even more preferably 0.37 ng/kg or less and 0.06 ng/kg or more, further preferably 0.3 ng/kg or less and 0.06 ng/kg or more, even further preferably 0.25 ng/kg or less and 0.12 ng/kg or more.

Preferably, the amount and/or total amounts of the IL-4 which the pharmaceutical compositions, the injectable dosage forms, the topical dosage forms, the medical devices and/or the kits of parts are suitable and/or configured to deliver, are as follows, namely:
IL-4 in an amount, preferably a total amount, of 20 ng/kg body mass of the subject or less, preferably per administration dose, to the skin of the subject. More preferably, IL-4 in an amount of 10 ng/kg or less, even more preferably 5 ng/kg or less, still more preferably 2.5 ng/kg or less, still even more preferably 1.5 ng/kg or less, further preferably 1 ng/kg or less, still even more preferably 0.4 ng/kg or less, even further preferably 0.2 ng/kg or less.
There is no lower limit for the amount, preferably the total amount, per kg body mass as long as the beneficial effects can be achieved, however, usually, a lower limit is 0.002 ng/kg or more, more preferably 0.025 ng/kg or more, even more preferably 0.01 ng/kg or more, still more preferably 0.02 mg/kg or more, still even more preferably 0.05 ng/kg or more, further preferably 0.075 ng/kg or more.
More preferably, IL-4 in an amount, preferably a total amount, in the range of 20 ng/kg or less and 0.002 ng/kg or more, even more preferably 10 ng/kg or less and 0.025 ng/kg or more, still more preferably 5 ng/kg or less and 0.01 ng/kg or more, still even more preferably 2.5 ng/kg or less and 0.01 ng/kg or more, further preferably 1.5 ng/kg or less and 0.02 ng/kg or more, even further preferably 1 ng/kg or less and 0.02 ng/kg or more, still further preferably 0.4 ng/kg or less and 0.05 ng/kg or more and still even further preferably 0.2 ng/kg or less and 0.075 ng/kg or more.

Preferably, the amount and/or total amounts of the BDNF which the pharmaceutical compositions, the injectable dosage forms, the topical dosage forms, the medical devices and/or the kits of parts are suitable and/or configured to deliver, are as follows, namely:
BDNF in an amount, preferably a total amount, of 10 ng/kg body mass of the subject or less, preferably per administration dose, to the skin of the subject. More preferably, BDNF in an amount of 5 ng/kg or less, even more preferably 2.5 ng/kg or less, still more preferably 1 ng/kg or less, still even more preferably 0.5 ng/kg or less, further preferably 0.3 ng/kg or less, still even more preferably 0.1 ng/kg or less, further preferably 0.05 ng/kg or less.
There is no lower limit for the amount, preferably the total amount, per kg body mass as long as the beneficial effects can be achieved, however, usually, a lower limit is 0.001 ng/kg or more, more preferably 0.005 ng/kg or more, even more preferably 0.007 ng/kg or more, still more preferably 0.01 mg/kg or more.
More preferably, BDNF in an amount, preferably a total amount, in the range of 10 ng/kg or less and 0.001 ng/kg or more, even more preferably 5 ng/kg or less and 0.001 ng/kg or more, still more preferably 2.5 ng/kg or less and 0.005 ng/kg or more, still even more preferably 1 ng/kg or less and 0.005 ng/kg or more, further preferably 0.5 ng/kg or less and 0.007 ng/kg or more, even further preferably 0.3 ng/kg or less and 0.007 ng/kg or more, still further preferably 0.1 ng/kg or less and 0.01 ng/kg or more, still even further preferably 0.05 ng/kg or less and 0.01 ng/kg or more.

Preferably, the amount of the IFN-γ, IL-4, BDNF and/or IL-2 to be delivered is the total amount.

Preferably, the amount of the IFN-γ, IL-4, BDNF and/or IL-2 is the amount delivered per administration dose.

More preferably, the total amount of the IFN-γ, IL-4, BDNF and/or IL-2 is the total amount delivered per administration dose.

Preferably, the pharmaceutical compositions, the injectable dosage forms, the topical dosage forms, the medical devices and/or the kits of parts are suitable and/or configured to deliver the IFN-γ-like acting substance(s), IL-4-like acting substance(s), BDNF-like acting substance(s) and/or IL-2-like acting substance(s), IFN-γ, IL-4, BDNF, IL-2 in an amount that does not exceed these amounts and/or total amounts. That is, the pharmaceutical compositions, the injectable dosage forms, the topical dosage forms, the medical devices and/or the kits of parts are suitable and/or configured to deliver not more than the amounts and/or total amounts as indicated.

Even more preferably, if not mentioned otherwise, itis to be understood that any description and definition of the amount and/or total amount of immunomodulatory substance(s), IFN-γ-like acting substance(s), IL-4-like acting substance(s), BDNF-like acting substance(s), IL-2-like acting substance(s), IFN-γ, IL-4, BDNF, IL-2 and/or the skin-conditioning agent(s) as preferably administered in the method as described herein, particularly in steps (A), (B), (B₁) and/or (C) of the method as described herein is independently and mutatis mutandis applicable to the amount of the immunomodulatory substance(s), IFN-γ-like acting substance(s), IL-4-like acting substance(s), BDNF-like acting substance(s) and/or IL-2-like acting substance(s), IFN-γ, IL-4, BDNF, IL-2 and/or the skin-conditioning agent(s) to be delivered by the pharmaceutical compositions, the injectable dosage forms, the topical dosage forms, the medical devices and/or the kits of parts as described herein. Hence, if not mentioned otherwise, it is to be understood that any embodiment or definition described herein in terms of the amount administered of the IFN-γ-like acting substance(s), IL-4-like acting substance(s), BDNF-like acting substance(s), IL-2-like acting substance(s), IFN-γ, IL-4, BDNF and/or IL-2, particularly in any of the embodiments of the method as described herein as described herein, more particularly of step (B), (B₁) and/or (C) of the method, is independently and mutatis mutandis applicable to the amounts to be delivered in any of the embodiments of the pharmaceutical compositions, the injectable dosage forms and/or the topical dosage forms described herein.

Even more preferably, the pharmaceutical compositions, the topical dosage forms, the medical devices and/or the injectable dosage forms are suitable and/or configured to deliver at least one of the IFN-γ-like acting substance(s), IL-4-like acting substance(s), BDNF-like acting substance(s), IL-2-like acting substance(s), IFN-γ, IL-4, BDNF, IL-2 in the above indicated amount(s), when one or more of the following conditions is fulfilled, namely when the pharmaceutical compositions, the topical dosage forms, the medical devices and/or the injectable dosage forms are:
administered or used for an administration to the skin of the subject;
preferably administered or used for an administration to the skin of the subject by topical application;
preferably administered or used for an administration to the skin of the subject by injection;
administered or used for an administration to the skin of a skin area;
preferably administered or used for an administration to the skin of the skin area, wherein an increased concentration of the IFN-γ, IL-4, BDNF and/or IL-2 is generated within the skin of the skin area;
preferably administered or used for an administration to the skin of the skin area by applying the pharmaceutical compositions, the topical dosage forms, the injectable dosage forms and/or the medical devices to the skin of an application area;
more preferably administered or used for an administration to the skin of the skin area by applying the pharmaceutical compositions, the topical dosage forms, the injectable dosage forms and/or the medical device to the skin of the application area, wherein the increased concentration of the IFN-γ, IL-4, BDNF and/or IL-2 is generated within the skin of the skin area;
applied to the skin of the application area;
preferably applied to the skin of the application area, wherein the increased concentration of the IFN-γ, IL-4, BDNF and/or IL-2 is generated within the skin of the skin area;
are applied to the skin of the application area by topical application; or
are applied to the skin of the application area by injection.

Furthermore, even more preferably, the pharmaceutical compositions, the topical dosage forms, the medical devices and/or the injectable dosage forms are suitable and/or configured to deliver at least one of the IFN-γ-like acting substance(s), IL-4-like acting substance(s), BDNF-like acting substance(s), IL-2-like acting substance(s), IFN-γ, IL-4, BDNF, IL-2 in the above indicated amount(s), when an increased concentration thereof, preferably an effective increased concentration thereof is generated.

It is to be understood that any description and definition of the generated increased concentration or the generated effective increased concentration IFN-γ-like acting substance(s), IL-4-like acting substance(s), BDNF-like acting substance(s), IL-2-like acting substance(s), IFN-γ, IL-4, BDNF, IL-2 as mentioned in any of the embodiments described herein, particularly of the method as described herein, more particularly of steps (B), (B₁) and/or (C) of the method as described herein is independently and mutatis mutandis applicable the pharmaceutical compositions, the topical dosage forms, the medical devices and/or the injectable dosage forms which are suitable and/or configured to deliver at least one of the IFN-γ-like acting substance(s), IL-4-like acting substance(s), BDNF-like acting substance(s), IL-2-like acting substance(s), IFN-γ, IL-4, BDNF, IL-2 in the above indicated amount(s), when an increased concentration thereof, preferably an effective increased concentration thereof is generated.

Preferably, it is to be understood that any description and definition of the skin area as mentioned in any of the embodiment of the method as described herein is independently and mutatis mutandis applicable to the skin area as mentioned in any of the embodiments of the pharmaceutical compositions, the injectable dosage forms, the topical dosage forms, the medical devices and/or the kits of parts described herein.

Preferably, it is to be understood that any description and definition of the application area as mentioned in any of the embodiment of the method as described herein is independently and mutatis mutandis applicable to the application area as mentioned in any of the embodiments of the pharmaceutical compositions, the injectable dosage forms, the topical dosage forms, the medical devices and/or the kits of parts described herein.

More preferably, in any of the embodiments of the pharmaceutical compositions, the injectable dosage forms, the topical dosage forms and/or the medical devices described herein, the skin area is in case of the substance(s) particularly in case of IFN-γ, IL-4 and BDNF, the skin area [b] and/or [b₁] and/or the application area is the application area [b] and/or [b₁] as mentioned in any of the embodiments described herein, particularly of step (B) and/or (B₁) of the method described herein.

More preferably, in any of the embodiments of the pharmaceutical compositions, the injectable dosage forms, the topical dosage forms and/or the medical devices described herein, the skin area is in case of the immunomodulatory substance(s), particularly in case of IL-2, the skin area [c] and/or the application area is the application area [c] as mentioned in any of the embodiments described herein, particularly of step (C) of the method described herein.

Preferably, in any of the embodiments of the pharmaceutical compositions, the injectable dosage forms, the topical dosage forms and/or the medical devices described herein, the skin area, the skin area [b], the skin area [b₁] and/or the skin area [c] are independently and mutatis mutandis defined the same as the skin area, the skin area [b] and/or the skin area [b₁] and/or the skin area [c], respectively, as mentioned in any of the embodiments described herein, particularly of step (B) and/or (B₁) of the method described herein. If not mentioned otherwise, it is to be understood that any embodiment or definition described herein in terms of the skin area, skin area [b], skin area [b₁] and/or skin area [c] is independently and mutatis mutandis applicable to the skin area, skin area [b], skin area [b₁] and/or skin area [c] in any of the embodiments of the pharmaceutical compositions, the injectable dosage forms, the topical dosage forms and/or the medical devices described herein.

Preferably, in any of the embodiments of the pharmaceutical compositions, the injectable dosage forms, the topical dosage forms and/or the medical devices described herein, the application area, the application area [b], the application area [b₁] and/or the application area [c] are independently and mutatis mutandis defined the same as the application area, the application area [b] and/or the application area [b₁] and/or the application area [c], respectively, as mentioned in any of the embodiments described herein, particularly of step (C) of the method described herein. If not mentioned otherwise, it is to be understood that any embodiment or definition described herein in terms of an application area, application area [b], application area [b₁] and/or application area [c] is independently and mutatis mutandis applicable to the application area, application area [b], application area [b₁] and/or application area [c] in any of the embodiments of the pharmaceutical compositions, the injectable dosage forms, the topical dosage forms and/or the medical devices described herein.

Preferably, where applicable, the pharmaceutical compositions, the injectable dosage forms, the topical dosage forms, the medical devices and/or the kits of parts comprise, preferably consist of, preferably as active ingredient(s):
IFN-γ-like acting substance(s) in a concentration equivalent to 100,000 IU IFN-γ/ml or IU IFN-γ/g or less, or in a concentration equivalent in activity to 5,000 ng IFN-γ/ml or ng IFN-γ/g or less;
IL-4-like acting substance(s) in a concentration or equivalent in activity to 1,000 ng IL-4/ml or ng/g or less;
BDNF-like acting substance(s) in a concentration equivalent in activity to 1,500 ng BDNF/ml or ng/g or less;
IL-2-like acting substance(s) in a concentration equivalent to 100,000 IU IL-2/ml or IU IL-2/g less, or in a concentration equivalent in activity to 6100 ng IL-2/ml or ng IL-2/g or less.

More preferably, the pharmaceutical compositions, the injectable dosage forms, the topical dosage forms, the medical devices and/or the kits of parts comprise, preferably consist of, IFN-γ-like acting substance in a concentration equivalent to 80,000 IU IFN-γ/ml or IU IFN-γ/g or less, even more preferably 70,000 IU IFN-γ/ml or IU IFN-γ/g or less, still more preferably 60,000 IU IFN-γ/ml or IU IFN-γ/g or less, still even more preferably 50,000 IU IFN-γ/ml or IU IFN-γ/g or less, further preferably 40,000 IU IFN-γ/ml or IU IFN-γ/g or less, even further preferably 30,000 IU IFN-γ/ml or IU IFN-γ/g or less, still further preferably 20,000 IU IFN-γ/ml or IU IFN-γ/g or less, still even further preferably 10,000 IU IFN-γ/ml or IU IFN-γ/g or less, furthermore preferably 5,000 IU IFN-γ/ml or IU IFN-γ/g or less, even furthermore preferably 1,000 IU IFN-γ/ml or IU IFN-γ/g or less. There is no lower limit for the concentration as long as the beneficial effects can be achieved, however, usually, a lower limit is a concentration equivalent to 2 IU IFN-γ/ml or IU IFN-γ/g or more, more preferably 10 IU IFN-γ/ml or IU IFN-γ/g or more, even more preferably 20 IU IFN-γ/ml or IU IFN-γ/g or more, still more preferably 30 IU IFN-γ/ml or IU IFN-γ/g or more and still even more preferably 50 IU IFN-γ/ml or IU IFN-γ/g or more. More preferably, the concentration of the IFN-γ-like acting substance is in the range of equivalent to 100,000 IU IFN-γ/ml or IU IFN-γ/g or less and 2 IU IFN-γ/ml or IU IFN-γ/g or more, even more preferably 80,000 IU IFN-γ/ml or IU IFN-γ/g or less and 2 IU IFN-γ/ml or IU IFN-γ/g or more, still more preferably 70,000 IU IFN-γ/ml or IU IFN-γ/g or less and 10 IU IFN-γ/ml or IU IFN-γ/g or more, still even more preferably 60,000 IU IFN-γ/ml or IU IFN-γ/g or less and 10 IU IFN-γ/ml or IU IFN-γ/g or more, further preferably 50,000 IU IFN-γ/ml or IU IFN-γ/g or less and 10 IU IFN-γ/ml or IU IFN-γ/g or more, even further preferably 40,000 IU IFN-γ/ml or IU IFN-γ/g or less and 20 IU IFN-γ/ml or IU IFN-γ/g or more, still further preferably 30,000 IU IFN-γ/ml or IU IFN-γ/g or less and 20 IU IFN-γ/ml or IU IFN-γ/g or more, still even further preferably 20,000 IU IFN-γ/ml or IU IFN-γ/g or less and IU IFN-γ/ml or IU IFN-γ/g or more, furthermore preferably 10,000 IU IFN-γ/ml or IU IFN-γ/g or less and 30 IU IFN-γ/ml or IU IFN-γ/g or more, even furthermore preferably 5,000 IU IFN-γ/ml or IU IFN-γ/g or less and 30 IU IFN-γ/ml or IU IFN-γ/g or more, still furthermore preferably 1,000 IU IFN-γ/ml or IU IFN-γ/g or less and 50 IU IFN-γ/ml or IU IFN-γ/g or more.

More preferably, the pharmaceutical compositions, the injectable dosage forms, the topical dosage forms, the medical devices and/or the kits of parts comprise, preferably consist of, IFN-γ-like acting substance in a concentration equivalent to 4,000 ng IFN-γ/ml or ng IFN-γ/g or less, even more preferably 3,500 ng IFN-γ/ml or ng IFN-γ/g or less, still more preferably 3,000 ng IFN-γ/ml or ng IFN-γ/g or less, still even more preferably 2,500 ng IFN-γ/ml or ng IFN-γ/g or less, further preferably 2,000 ng IFN-γ/ml or ng IFN-γ/g or less, even further preferably 1,500 ng IFN-γ/ml or ng IFN-γ/g or less, still further preferably 1,000 ng IFN-γ/ml or ng IFN-γ/g or less, still even further preferably 500 ng IFN-γ/ml or ng IFN-γ/g or less, furthermore preferably 250 ng IFN-γ/ml or ng IFN-γ/g or less, even furthermore preferably 50 ng IFN-γ/ml or ng IFN-γ/g or less. There is no lower limit for the concentration as long as the beneficial effects can be achieved, however, usually, a lower limit is a concentration equivalent to 0.1 ng IFN-γ/ml or ng IFN-γ/g or more, more preferably 0.5 ng IFN-γ/ml or ng IFN-γ/g or more, even more preferably 1 ng IFN-γ/ml or ng IFN-γ/g or more and still even more preferably 2 ng IFN-γ/ml or ng IFN-γ/g or more. More preferably, the concentration of the IFN-γ-like acting substance is in the range of equivalent to 5,000 ng IFN-γ/ml or ng IFN-γ/g or less and is 0.1 ng IFN-γ/ml or ng IFN-γ/g or more, even more preferably 4,000 ng IFN-γ/ml or ng IFN-γ/g or less and is 0.1 ng IFN-γ/ml or ng IFN-γ/g or more, still more preferably 3,500 ng IFN-γ/ml or ng IFN-γ/g or less and 0.1 ng IFN-γ/ml or ng IFN-γ/g or more, still even more preferably 3,000 ng IFN-γ/ml or ng IFN-γ/g or less and 0.5 ng IFN-γ/ml or ng IFN-γ/g or more, further preferably 2,500 ng IFN-γ/ml or ng IFN-γ/g or less and 0.5 ng IFN-γ/ml or ng IFN-γ/g or more, even further preferably 2,000 ng IFN-γ/ml or ng IFN-γ/g or less and 0.5 ng IFN-γ/ml or ng IFN-γ/g or more, still further preferably 1,500 ng IFN-γ/ml or ng IFN-γ/g or less and 1 ng IFN-γ/ml or ng IFN-γ/g or more, still even further preferably 1,000 ng IFN-γ/ml or ng IFN-γ/g or less and 1 ng IFN-γ/ml or ng IFN-γ/g or more, furthermore preferably 500 ng IFN-γ/ml or ng IFN-γ/g or less and 1 ng IFN-γ/ml or ng IFN-γ/g or more, even furthermore preferably 250 ng IFN-γ/ml or ng IFN-γ/g or less and 2 ng IFN-γ/ml or ng IFN-γ/g or more, still furthermore preferably 50 ng IFN-γ/ml or ng IFN-γ/g or less and 2 ng IFN-γ/ml or ng IFN-γ/g or more.

The same amounts apply to any derivative, fragment, protein-analogue, peptide, protein-variant, biopharmaceutical, inductor, precursor, prodrug, mutein, co-drug, propeptide and/or any pharmaceutically acceptable salt of the IFN-γ.

More preferably, the pharmaceutical compositions, the injectable dosage forms, the topical dosage forms, the medical devices and/or the kits of parts comprise, preferably consist of IL-4-like acting substance in a concentration equivalent in activity to 8,000 ng IL-4/ml or ng IL-4/g or less, even more preferably 7,000 ng IL-4/ml or ng IL-4/g or less, still more preferably 6,000 ng IL-4/ml or ng IL-4/g or less, still even more preferably 5,000 ng IL-4/ml or ng IL-4/g or less, further preferably 4,000 ng IL-4/ml or ng IL-4/g or less, even further preferably 3,000 ng IL-4/ml or ng IL-4/g or less, still further preferably 2,000 ng IL-4/ml or ng IL-4/g or less, still even further preferably 1,000 ng IL-4/ml or ng IL-4/g or less, furthermore preferably 500 ng IL-4/ml or ng IL-4/g or less, even furthermore preferably 150 ng IL-4/ml or ng IL-4/g or less. There is no lower limit for the concentration as long as the beneficial effects can be achieved, however, usually, a lower limit is a concentration equivalent in activity to 0.1 ng IL-4/ml or ng IL-4/g or more, more preferably 0.5 ng IL-4/ml or ng IL-4/g or more, even more preferably 1 ng IL-4/ml or ng IL-4/g or more, still more preferably 1 ng IL-4/ml or ng IL-4/g or more and still even more preferably 2 ng IL-4/ml or ng IL-4/g or more. More preferably, the concentration of the IL-4-like acting substance is in the range of equivalent in activity to 10,000 ng IL-4/ml or ng IL-4/g or less and 0.1 ng IL-4/ml or ng IL-4/g or more, even more preferably 8,000 ng IL-4/ml or ng IL-4/g or less and 0.1 ng IL-4/ml or ng IL-4/g or more, still more preferably 7,000 ng IL-4/ml or ng IL-4/g or less and 0.1 ng IL-4/ml or ng IL-4/g or more, still even more preferably 6,000 ng IL-4/ml or ng IL-4/g or less and 0.5 ng IL-4/ml or ng IL-4/g or more, further preferably 5,000 ng IL-4/ml or ng IL-4/g or less and 0.5 ng IL-4/ml or ng IL-4/g or more, even further preferably 4,000 ng IL-4/ml or ng IL-4/g or less and 0.5 ng IL-4/ml or ng IL-4/g or more, still further preferably 3,000 ng IL-4/ml or ng IL-4/g or less and 1 ng IL-4/ml or ng IL-4/g or more, still even further preferably 2,000 ng IL-4/ml or ng IL-4/g or less and 1 ng IL-4/ml or ng IL-4/g or more, furthermore preferably 1,000 ng IL-4/ml or ng IL-4/g or less and 1 ng IL-4/ml or ng IL-4/g or more, even furthermore preferably 500 ng IL-4/ml or ng IL-4/g or less and 2 ng IL-4/ml or ng IL-4/g or more, still furthermore preferably 150 ng IL-4/ml or ng IL-4/g or less and 2 ng IL-4/ml or ng IL-4/g or more.

The same amounts apply to any derivative, fragment, protein-analogue, peptide, protein-variant, biopharmaceutical, inductor, precursor, prodrug, mutein, co-drug, propeptide and/or any pharmaceutically acceptable salt of the IL-2.

More preferably, the pharmaceutical compositions, the injectable dosage forms, the topical dosage forms, the medical devices and/or the kits of parts comprise, preferably consist of, BDNF-like acting substance in a concentration equivalent in activity to 8,000 ng BDNF/ml or ng BDNF/g or less, even more preferably 7,000 ng BDNF/ml or ng BDNF/g or less, still more preferably 6,000 ng BDNF/ml or ng BDNF/g or less, still even more preferably 5,000 ng BDNF/ml or ng BDNF/g or less, further preferably 4,000 ng BDNF/ml or ng BDNF/g or less, even further preferably 3,000 ng BDNF/ml or ng BDNF/g or less, still further preferably 2,000 ng BDNF/ml or ng BDNF/g or less, still even further preferably 1,000 ng BDNF/ml or ng BDNF/g or less, furthermore preferably 500 ng BDNF/ml or ng BDNF/g or less, even furthermore preferably 150 ng BDNF/ml or ng BDNF/g or less, still furthermore preferably 50 ng BDNF/ml or ng BDNF/g or less. There is no lower limit for the concentration as long as the beneficial effects can be achieved, however, usually, a lower limit is a concentration equivalent in activity to 0.005 ng BDNF/ml or ng BDNF/g or more, more preferably 0.01 ng BDNF/ml or ng BDNF/g or more, even more preferably 0.05 ng BDNF/ml or ng BDNF/g or more, still even more preferably 0.1 ng BDNF/ml or ng BDNF/g or more, further preferably 0.2 ng BDNF/ml or ng BDNF/g or more. More preferably, the concentration of the BDNF-like acting substance is in the range of in a concentration of equivalent in activity to 10,000 ng BDNF/ml or ng BDNF/g or less and 0.005 ng BDNF/ml or ng BDNF/g or more, even more preferably 8,000 ng BDNF/ml or ng BDNF/g or less and 0.005 ng BDNF/ml or ng BDNF/g or more, still more preferably 7,000 ng BDNF/ml or ng BDNF/g or less and 0.01 ng BDNF/ml or ng BDNF/g or more, still even more preferably 6,000 ng BDNF/ml or ng BDNF/g or less and 0.01 ng BDNF/ml or ng BDNF/g or more, further preferably 5,000 ng BDNF/ml or ng BDNF/g or less and 0.01 ng BDNF/ml or ng BDNF/g or more, even further preferably 4,000 ng BDNF/ml or ng BDNF/g or less and 0.05 ng BDNF/ml or ng BDNF/g or more, still further preferably 3,000 ng BDNF/ml or ng BDNF/g or less and 0.5 ng BDNF/ml or ng BDNF/g or more, still even further preferably 2,000 ng BDNF/ml or ng BDNF/g or less and 0.5 ng BDNF/ml or ng BDNF/g or more, furthermore preferably 1,000 ng BDNF/ml or ng BDNF/g or less and 0.1 ng BDNF/ml or ng BDNF/g or more, even furthermore preferably 500 ng BDNF/ml or ng BDNF/g or less and 0.1 ng BDNF/ml or ng BDNF/g or more, still furthermore preferably 150 ng BDNF/ml or ng BDNF/g or less and 0.2 ng BDNF/ml or ng BDNF/g or more, still even furthermore preferably 50 ng BDNF/ml or ng BDNF/g or less and 0.2 ng BDNF/ml or ng BDNF/g or more.

The same amounts apply to any derivative, fragment, protein-analogue, peptide, protein-variant, biopharmaceutical, inductor, precursor, prodrug, mutein, co-drug, propeptide and/or any pharmaceutically acceptable salt of the BDNF.

More preferably, the pharmaceutical compositions, the injectable dosage forms, the topical dosage forms, the medical devices and/or the kits of parts comprise, preferably consist of, IL-2-like acting substance in a concentration equivalent to 80,000 IU IL-2/ml or IU IL-2/g or less, even more preferably 70,000 IU IL-2/ml or IU IL-2/g or less, still more preferably 60,000 IU IL-2/ml or IU IL-2/g or less, still even more preferably 50,000 IU IL-2/ml or IU IL-2/g or less, further preferably 40,000 IU IL-2/ml or IU IL-2/g or less, even further preferably 30,000 IU IL-2/ml or IU IL-2/g or less, still further preferably 20,000 IU IL-2/ml or IU IL-2/g or less, still even further preferably 10,000 IU IL-2/ml or IU IL-2/g or less, furthermore preferably 5,000 IU IL-2/ml or IU IL-2/g or less, even furthermore preferably 2,000 IU IL-2/ml or IU IL-2/g or less. There is no lower limit for the concentration as long as the beneficial effects can be achieved, however, usually, a lower limit is a concentration equivalent to 25 IU IL-2/ml or IU IL-2/g or more, more preferably 50 IU IL-2/ml or IU IL-2/g or more, even more preferably 100 IU IL-2/ml or IU IL-2/g or more. More preferably, the concentration of the IL-2-like acting substance is in the range of equivalent to 100,000 IU IL-2/ml or IU IL-2/g or less and IU IL-2/ml or IU IL-2/g or more, even more preferably 80,000 IU IL-2/ml or IU IL-2/g or less and 25 IU IL-2/ml or IU IL-2/g or more, still more preferably 70,000 IU IL-2/ml or IU IL-2/g or less and 25 IU IL-2/ml or IU IL-2/g or more, still even more preferably 60,000 IU IL-2/ml or IU IL-2/g or less and 25 IU IL-2/ml or IU IL-2/g or more, further preferably 50,000 IU IL-2/ml or IU IL-2/g or less and 50 IU IL-2/ml or IU IL-2/g or more, even further preferably 40,000 IU IL-2/ml or IU IL-2/g or less and 50 IU IL-2/ml or IU IL-2/g or more, still further preferably 30,000 IU IL-2/ml or IU IL-2/g or less and 50 IU IL-2/ml or IU IL-2/g or more, still even further preferably 20,000 IU IL-2/ml or IU IL-2/g or less and 50 IU IL-2/ml or IU IL-2/g or more, furthermore preferably 10,000 IU IL-2/ml or IU IL-2/g or less and 50 IU IL-2/ml or IU IL-2/g or more, even furthermore preferably 5,000 IU IL-2/ml or IU IL-2/g or less and 100 IU IL-2/ml or IU IL-2/g or more, still furthermore preferably 1,000 IU IL-2/ml or IU IL-2/g or less and 100 IU IL-2/ml or IU IL-2/g or more.

More preferably, the pharmaceutical compositions, the injectable dosage forms, the topical dosage forms, the medical devices and/or the kits of parts comprise, preferably consist of, IL-2-like acting substance in a concentration equivalent in activity to 4,880 ng IL-2/ml or ng IL-2/g, even more preferably 4,270 ng IL-2/ml or ng IL-2/g or less, still more preferably 3,660 ng IL-2/ml or ng IL-2/g or less, still even more preferably 3,050 ng IL-2/ml or ng IL-2/g or less, further preferably 2,440 ng IL-2/ml or ng IL-2/g or less, even further preferably 1,830 ng IL-2/ml or ng IL-2/g or less, still further preferably 1,220 ng IL-2/ml or ng IL-2/g or less, still even further preferably 610 ng IL-2/ml or ng IL-2/g or less, furthermore preferably 300 ng IL-2/ml or ng IL-2/g or less, even furthermore preferably 120 ng IL-2/ml or ng IL-2/g or less. There is no lower limit for the concentration as long as the beneficial effects can be achieved, however, usually, a lower limit is a concentration equivalent in activity to 2 ng IL-2/ml or ng IL-2/g or more, more preferably 3 ng IL-2/ml or ng IL-2/g or more, even more preferably 6.1 ng IL-2/ml or ng IL-2/g or more. More preferably, the concentration of the IL-2-like acting substance is in the range of equivalent in activity to 6,100 ng IL-2/ml or ng IL-2/g and 2 ng IL-2/ml or ng IL-2/g or more, even more preferably 4,880 ng IL-2/ml or ng IL-2/g and 2 ng IL-2/ml or ng IL-2/g or more, still more preferably 4,270 ng IL-2/ml or ng IL-2/g or less and 2 ng IL-2/ml or ng IL-2/g or more, still even more preferably 3,660 ng IL-2/ml or ng IL-2/g or less and 2 ng IL-2/ml or ng IL-2/g or more, further preferably 3,050 ng IL-2/ml or ng IL-2/g or less and 3 ng IL-2/ml or ng IL-2/g or more, even further preferably 2,440 ng IL-2/ml or ng IL-2/g or less and 3 ng IL-2/ml or ng IL-2/g or more, still further preferably 1,830 ng IL-2/ml or ng IL-2/g or less and 3 g/ml or ng IL-2/g or more, still even further preferably 1,220 ng IL-2/ml or ng IL-2/g or less and 3 ng IL-2/ml or ng IL-2/g or more, furthermore preferably 610 ng IL-2/ml or ng IL-2/g or less and 6.1 ng IL-2/ml or ng IL-2/g or more, even furthermore preferably 300 ng IL-2/ml or ng IL-2/g or less and 6.1 ng IL-2/ml or ng IL-2/g or more, still furthermore preferably 120 ng IL-2/ml or ng IL-2/g or less and 6.1 ng IL-2/ml or ng IL-2/g or more.

The same amounts apply to any derivative, fragment, protein-analogue, peptide, protein-variant, biopharmaceutical, inductor, precursor, prodrug, mutein, co-drug, propeptide and/or any pharmaceutically acceptable salt of the IL-2.

Preferably, the IFN-γ-like acting substance is IFN-γ and/or any derivative, fragment, protein-analogue, peptide, protein-variant, biopharmaceutical, inductor, precursor, prodrug, mutein, co-drug, propeptide thereof and/or any pharmaceutically acceptable salt of any of these,
the IL-4-like acting substance is IL-4 and/or any derivative, fragment, protein-analogue, peptide, protein-variant, biopharmaceutical, inductor, precursor, prodrug, mutein, co-drug, propeptide thereof and/or any pharmaceutically acceptable salt of any of these,
the BDNF-like acting substance is BDNF and/or any derivative, fragment, protein-analogue, peptide, protein-variant, biopharmaceutical, inductor, precursor, prodrug, mutein, co-drug, propeptide thereof and/or any pharmaceutically acceptable salt of any of these,
the IL-2-like acting substance is IL-2 and/or any derivative, fragment, protein-analogue, peptide, protein-variant, biopharmaceutical, inductor, precursor, prodrug, mutein, co-drug, propeptide thereof and/or any pharmaceutically acceptable salt of any of these, still more preferably IFN-γ, IL-4, BDNF and/or IL-2.

Hence, still more preferably, where applicable, the pharmaceutical compositions, the injectable dosage forms, the topical dosage forms, the medical devices and/or the kits of parts comprise, preferably consist of, preferably as active ingredient(s):
IFN-γ in a concentration of 100,000 IU/ml or IU/g or less, or of 5,000 ng/ml or ng/g or less;
IL-4 in a concentration of 10,000 ng/ml or ng/g or less;
BDNF in a concentration of 10,000 ng/ml or ng/g or less; and/or
IL-2 in a concentration of 100,000 IU/ml or IU/g less, or of 6,100 ng/ml or ng/g or less.

More preferably, the pharmaceutical compositions, the injectable dosage forms, the topical dosage forms, the medical devices and/or the kits of parts comprise, preferably consist of, IFN-γ in a concentration of 80,000 IU/ml or IU/g or less, even more preferably 70,000 IU/ml or IU/g or less, still more preferably 60,000 IU/ml or IU/g or less, still even more preferably 50,000 IU/ml or IU/g or less, further preferably 40,000 IU/ml or IU/g or less, even further preferably 30,000 IU/ml or IU/g or less, still further preferably 20,000 IU/ml or IU/g or less, still even further preferably 10,000 IU/ml or IU/g or less, furthermore preferably 5,000 IU/ml or IU/g or less, even furthermore preferably 1,000 IU/ml or IU/g or less. There is no lower limit for the concentration as long as the beneficial effects can be achieved, however, usually, a lower limit is 2 IU/ml or IU/g or more, more preferably 10 IU/ml or IU/g or more, even more preferably 20 IU/ml or IU/g or more, still more preferably 30 IU/ml or IU/g or more and still even more preferably 50 IU/ml or IU/g or more. More preferably, the concentration of the IFN-γ is in the range of 100,000 IU/ml or IU/g or less and 2 IU/ml or IU/g or more, even more preferably 80,000 IU/ml or IU/g or less and 2 IU/ml or IU/g or more, still more preferably 70,000 IU/ml or IU/g or less and IU/ml or IU/g or more, still even more preferably 60,000 IU/ml or IU/g or less and 10 IU/ml or IU/g or more, further preferably 50,000 IU/ml or IU/g or less and 10 IU/ml or IU/g or more, even further preferably 40,000 IU/ml or IU/g or less and 20 IU/ml or IU/g or more, still further preferably 30,000 IU/ml or IU/g or less and 20 IU/ml or IU/g or more, still even further preferably 20,000 IU/ml or IU/g or less and 20 IU/ml or IU/g or more, furthermore preferably 10,000 IU/ml or IU/g or less and 30 IU/ml or IU/g or more, even furthermore preferably 5,000 IU/ml or IU/g or less and 30 IU/ml or IU/g or more, still furthermore preferably 1,000 IU/ml or IU/g or less and 50 IU/ml or IU/g or more.

More preferably, the pharmaceutical compositions, the injectable dosage forms, the topical dosage forms, the medical devices and/or the kits of parts comprise, preferably consist of, IFN-γ in a concentration of 4,000 ng/ml or ng/g or less, even more preferably 3,500 ng/ml or ng/g or less, still more preferably 3,000 ng/ml or ng/g or less, still even more preferably 2,500 ng/ml or ng/g or less, further preferably 2,000 ng/ml or ng/g or less, even further preferably 1,500 ng/ml or ng/g or less, still further preferably 1,000 ng/ml or ng/g or less, still even further preferably 500 ng/ml or ng/g or less, furthermore preferably 250 ng/ml or ng/g or less, even furthermore preferably 50 ng/ml or ng/g or less. There is no lower limit for the concentration as long as the beneficial effects can be achieved, however, usually, a lower limit is 0.1 ng/ml or ng/g or more, more preferably 0.5 ng/ml or ng/g or more, even more preferably 1 ng/ml or ng/g or more and still even more preferably 2 ng/ml or ng/g or more. More preferably, the concentration of the IFN-γ is in the range of 5,000 ng/ml or ng/g or less and is 0.1 ng/ml or ng/g or more, even more preferably 4,000 ng/ml or ng/g or less and is 0.1 ng/ml or ng/g or more, still more preferably 3,500 ng/ml or ng/g or less and 0.1 ng/ml or ng/g or more, still even more preferably 3,000 ng/ml or ng/g or less and 0.5 ng/ml or ng/g or more, further preferably 2,500 ng/ml or ng/g or less and 0.5 ng/ml or ng/g or more, even further preferably 2,000 ng/ml or ng/g or less and 0.5 ng/ml or ng/g or more, still further preferably 1,500 ng/ml or ng/g or less and 1 ng/ml or ng/g or more, still even further preferably 1,000 ng/ml or ng/g or less and 1 ng/ml or ng/g or more, furthermore preferably 500 ng/ml or ng/g or less and 1 ng/ml or ng/g or more, even furthermore preferably 250 ng/ml or ng/g or less and 2 ng/ml or ng/g or more, still furthermore preferably 50 ng/ml or ng/g or less and 2 ng/ml or ng/g or more.

More preferably, the pharmaceutical compositions, the injectable dosage forms, the topical dosage forms, the medical devices and/or the kits of parts comprise, preferably consist of IL-4 in a concentration of 8,000 ng/ml or ng/g or less, even more preferably 7,000 ng/ml or ng/g or less, still more preferably 6,000 ng/ml or ng/g or less, still even more preferably 5,000 ng/ml or ng/g or less, further preferably 4,000 ng/ml or ng/g or less, even further preferably 3,000 ng/ml or ng/g or less, still further preferably 2,000 ng/ml or ng/g or less, still even further preferably 1,000 ng/ml or ng/g or less, furthermore preferably 500 ng/ml or ng/g or less, even furthermore preferably 150 ng/ml or ng/g or less. There is no lower limit for the concentration as long as the beneficial effects can be achieved, however, usually, a lower limit is 0.1 ng/ml or ng/g or more, more preferably 0.5 ng/ml or ng/g or more, even more preferably 1 ng/ml or ng/g or more, still more preferably 1 ng/ml or ng/g or more and still even more preferably 2 ng/ml or ng/g or more. More preferably, the concentration of the IL-4 is in the range of 10,000 ng/ml or ng/g or less and 0.1 ng/ml or ng/g or more, even more preferably 8,000 ng/ml or ng/g or less and 0.1 ng/ml or ng/g or more, still more preferably 7,000 ng/ml or ng/g or less and 0.1 ng/ml or ng/g or more, still even more preferably 6,000 ng/ml or ng/g or less and 0.5 ng/ml or ng/g or more, further preferably 5,000 ng/ml or ng/g or less and 0.5 ng/ml or ng/g or more, even further preferably 4,000 ng/ml or ng/g or less and 0.5 ng/ml or ng/g or more, still further preferably 3,000 ng/ml or ng/g or less and 1 ng/ml or ng/g or more, still even further preferably 2,000 ng/ml or ng/g or less and 1 ng/ml or ng/g or more, furthermore preferably 1,000 ng/ml or ng/g or less and 1 ng/ml or ng/g or more, even furthermore preferably 500 ng/ml or ng/g or less and 2 ng/ml or ng/g or more, still furthermore preferably 150 ng/ml or ng/g or less and 2 ng/ml or ng/g or more.

More preferably, the pharmaceutical compositions, the injectable dosage forms, the topical dosage forms, the medical devices and/or the kits of parts comprise, preferably consist of, BDNF in a concentration of 8,000 ng/ml or ng/g or less, even more preferably 7,000 ng/ml or ng/g or less, still more preferably 6,000 ng/ml or ng/g or less, still even more preferably 5,000 ng/ml or ng/g or less, further preferably 4,000 ng/ml or ng/g or less, even further preferably 3,000 ng/ml or ng/g or less, still further preferably 2,000 ng/ml or ng/g or less, still even further preferably 1,000 ng/ml or ng/g or less, furthermore preferably 500 ng/ml or ng/g or less, even furthermore preferably 150 ng/ml or ng/g or less, still furthermore preferably 50 ng/ml or ng/g or less. There is no lower limit for the concentration as long as the beneficial effects can be achieved, however, usually, a lower limit is 0.005 ng/ml or ng/g or more, more preferably 0.01 ng/ml or ng/g or more, even more preferably 0.05 ng/ml or ng/g or more, still even more preferably 0.1 ng/ml or ng/g or more, further preferably 0.2 ng/ml or ng/g or more. More preferably, the concentration of the BDNF is in the range of in a concentration of 10,000 ng/ml or ng/g or less and 0.005 ng/ml or ng/g or more, even more preferably 8,000 ng/ml or ng/g or less and 0.005 ng/ml or ng/g or more, still more preferably 7,000 ng/ml or ng/g or less and 0.01 ng/ml or ng/g or more, still even more preferably 6,000 ng/ml or ng/g or less and 0.01 ng/ml or ng/g or more, further preferably 5,000 ng/ml or ng/g or less and 0.01 ng/ml or ng/g or more, even further preferably 4,000 ng/ml or ng/g or less and 0.05 ng/ml or ng/g or more, still further preferably 3,000 ng/ml or ng/g or less and 0.5 ng/ml or ng/g or more, still even further preferably 2,000 ng/ml or ng/g or less and 0.5 ng/ml or ng/g or more, furthermore preferably 1,000 ng/ml or ng/g or less and 0.1 ng/ml or ng/g or more, even furthermore preferably 500 ng/ml or ng/g or less and 0.1 ng/ml or ng/g or more, still furthermore preferably 150 ng/ml or ng/g or less and 0.2 ng/ml or ng/g or more, still even furthermore preferably 50 ng/ml or ng/g or less and 0.2 ng/ml or ng/g or more.

More preferably, the pharmaceutical compositions, the injectable dosage forms, the topical dosage forms, the medical devices and/or the kits of parts comprise, preferably consist of, IL-2 in a concentration of 80,000 IU/ml or IU/g or less, even more preferably 70,000 IU/ml or IU/g or less, still more preferably 60,000 IU/ml or IU/g or less, still even more preferably 50,000 IU/ml or IU/g or less, further preferably 40,000 IU/ml or IU/g or less, even further preferably 30,000 IU/ml or IU/g or less, still further preferably 20,000 IU/ml or IU/g or less, still even further preferably 10,000 IU/ml or IU/g or less, furthermore preferably 5,000 IU/ml or IU/g or less, even furthermore preferably 2,000 IU/ml or IU/g or less. There is no lower limit for the concentration as long as the beneficial effects can be achieved, however, usually, a lower limit is 25 IU/ml or IU/g or more, more preferably 50 IU/ml or IU/g or more, even more preferably 100 IU/ml or IU/g or more. More preferably, the concentration of the IL-2 is in the range of 100,000 IU/ml or IU/g or less and 25 IU/ml or IU/g or more, even more preferably 80,000 IU/ml or IU/g or less and 25 IU/ml or IU/g or more, still more preferably 70,000 IU/ml or IU/g or less and 25 IU/ml or IU/g or more, still even more preferably 60,000 IU/ml or IU/g or less and 25 IU/ml or IU/g or more, further preferably 50,000 IU/ml or IU/g or less and 50 IU/ml or IU/g or more, even further preferably 40,000 IU/ml or IU/g or less and 50 IU/ml or IU/g or more, still further preferably 30,000 IU/ml or IU/g or less and 50 IU/ml or IU/g or more, still even further preferably 20,000 IU/ml or IU/g or less and 50 IU/ml or IU/g or more, furthermore preferably 10,000 IU/ml or IU/g or less and 50 IU/ml or IU/g or more, even furthermore preferably 5,000 IU/ml or IU/g or less and 100 IU/ml or IU/g or more, still furthermore preferably 1,000 IU/ml or IU/g or less and 100 IU/ml or IU/g or more.

More preferably, the pharmaceutical compositions, the injectable dosage forms, the topical dosage forms, the medical devices and/or the kits of parts comprise, preferably consist of, IL-2 in a concentration of 4,880 ng/ml or ng/g, even more preferably 4,270 ng/ml or ng/g or less, still more preferably 3,660 ng/ml or ng/g or less, still even more preferably 3,050 ng/ml or ng/g or less, further preferably 2,440 ng/ml or ng/g or less, even further preferably 1,830 ng/ml or ng/g or less, still further preferably 1,220 ng/ml or ng/g or less, still even further preferably 610 ng/ml or ng/g or less, furthermore preferably 300 ng/ml or ng/g or less, even furthermore preferably 120 ng/ml or ng/g or less. There is no lower limit for the concentration as long as the beneficial effects can be achieved, however, usually, a lower limit is 2 ng/ml or ng/g or more, more preferably 3 ng/ml or ng/g or more, even more preferably 6.1 ng/ml or ng/g or more. More preferably, the concentration of the IL-2 is in the range of 6,100 ng/ml or ng/g and 2 ng/ml or ng/g or more, even more preferably 4,880 ng/ml or ng/g and 2 ng/ml or ng/g or more, still more preferably 4,270 ng/ml or ng/g or less and 2 ng/ml or ng/g or more, still even more preferably 3,660 ng/ml or ng/g or less and 2 ng/ml or ng/g or more, further preferably 3,050 ng/ml or ng/g or less and 3 ng/ml or ng/g or more, even further preferably 2,440 ng/ml or ng/g or less and 3 ng/ml or ng/g or more, still further preferably 1,830 ng/ml or ng/g or less and 3 g/ml or ng/g or more, still even further preferably 1,220 ng/ml or ng/g or less and 3 ng/ml or ng/g or more, furthermore preferably 610 ng/ml or ng/g or less and 6.1 ng/ml or ng/g or more, even furthermore preferably 300 ng/ml or ng/g or less and 6.1 ng/ml or ng/g or more, still furthermore preferably 120 ng/ml or ng/g or less and 6.1 ng/ml or ng/g or more.

Preferably, where applicable, the pharmaceutical compositions, the injectable dosage forms, the topical dosage forms, the medical devices and/or the kits of parts further comprise, preferably consists of, a skin-conditioning agent, preferably as further active ingredient(s).

The skin-conditioning agent of the pharmaceutical compositions, the injectable dosage forms, the topical dosage forms, the medical devices and/or the kit of parts is any skin-conditioning agent as mentioned in any of the embodiments described herein, particularly of the method described herein, more particularly in step (A-7) of the method.

If not mentioned otherwise, it is to be understood that any embodiment or definition described herein in terms of the skin-conditioning agent, particularly in relation to the method, more particularly in step (A-7) of the method, is independently and mutatis mutandis applicable to the skin-conditioning agent of the pharmaceutical compositions, the injectable dosage forms, the topical dosage forms, the medical devices and/or the kit of parts as mentioned in any of the embodiments described herein.

Preferably, the skin-conditioning agent comprises as active ingredient(s), preferably consists of as active ingredient(s) of, nitrates, alpha blockers, ACE-inhibitors, ginkgo preparations like gingko balm, calcium antagonists, dihydralazine, minoxidil, dihydroergotoxin, nicotinic acid analogues, vasodilators like methylnicotinat, moxa herbs, capsaicine, pentoxifylline, vasodilator containing crème, methylnicotinat containing crème, Kytta® heat balm or any combination thereof, more preferably methylnicotinat containing crème and/or methylnicotinat, even more preferably Kytta® heat balm and/or methylnicotinat (Kytta® heat balm containing methylnicotinat [“Kytta® Heat balm” by P&G Health Germany GmbH, Germany, PZN 12358936]).

Preferably, in the injectable dosage forms, the topical dosage forms, the medical devices and/or the kits of parts, the skin-conditioning agent(s) is in the form of a pharmaceutical composition, more preferably in the form of any of the pharmaceutical compositions (I), (II), (III) and/or (IV) described herein.

Preferably, the injectable dosage forms, the topical dosage forms, the medical devices and/or the kits of parts comprise a pharmaceutical composition, comprising preferably consisting of, the skin-conditioning agent(s), preferably as active ingredient(s). More preferably, the pharmaceutical composition comprised in the injectable dosage form, topical dosage forms, the medical devices and/or the kits of parts is any of the pharmaceutical compositions (I), (II), (III) and/or (IV) described herein.

Preferably, the pharmaceutical compositions of the injectable dosage forms, the topical dosage forms, the medical devices and/or the kits of parts comprises, preferably consists of, the skin-conditioning agent(s), preferably as further active ingredient(s). More preferably, the pharmaceutical composition comprised in the injectable dosage forms, the topical dosage forms, the medical devices and/or the kits of parts is any of the pharmaceutical compositions (I), (II), (III) and/or (IV) described herein.

In any of the embodiments described herein and particularly the pharmaceutical compositions of the injectable dosage forms, the topical dosage forms, the medical devices and/or the kits of parts, where applicable, the skin-conditioning agent may be any blood-circulation-increasing agent, vasodilating agent, skin-temperature increasing agent, skin-502-increasing agent and/or skin-rHb-increasing agent.

Preferably, the pharmaceutical compositions, the injectable dosage forms, topical dosage forms, the medical devices and/or the kits of parts are suitable and/or configured to deliver an effective amount of the skin-conditioning agent(s). Particularly, the pharmaceutical compositions, the injectable dosage forms, the topical dosage forms, the medical devices and/or the kits of parts comprise and/or are suitable and/or configured to deliver an effective amount of the skin-conditioning agent(s).

Preferably, it is to be understood that any description and definition of the amount or the effective amount of the skin-conditioning agent(s) as preferably administered in the method to the skin of the subject, particularly in step (A-7) of the method as described herein is independently and mutatis mutandis applicable to the amount or the effective amount of the skin-conditioning agent as mentioned in any of the embodiments of the pharmaceutical compositions, the injectable dosage forms, the topical dosage forms, the medical devices and/or the kits of parts described herein which the amount or the effective amount they are suitable and/or configured to deliver.

More preferably the skin-conditioning agent is Kytta® heat balm and/or methylnicotinat. Hence, the pharmaceutical compositions, the injectable dosage forms, the topical dosage forms, the medical devices and/or the kits of parts are suitable and/or configured to deliver methylnicotinat in an amount corresponding to an amount of 5 mg to 10,000 mg, more preferably 10 mg to 1000 mg, even more preferably 15 mg to 500 mg Kytta® crème.

Preferably, the pharmaceutical compositions, topical dosage forms, the medical devices, the injectable dosage forms and/or the kits of parts are suitable and/or configured for generating:
an increased temperature on the skin;
an increased sO₂ (oxygen saturation of haemoglobin) within the skin; or
an increased rHb (relative haemoglobin amount) within the skin,
more preferably for generating:
an increased temperature on the skin;
an increased sO₂ within the skin; and/or
an increased rHb within the skin,
even more preferably all of an increased temperature, sO₂ and rHb.

More preferably,
the pharmaceutical compositions, the injectable dosage forms, the topical dosage forms, the medical devices and/or the kits of parts generate and/or are suitable and/or configured to generate;
the skin-conditioning agent(s) generates and/or is suitable and/or configured to generate;
the energizing topical dosage forms generate and/or are suitable and/or configured to generate; and/or
the topical dosage forms comprising a skin-conditioning agent generate and/or are suitable and/or configured to generate; and/or
the skin-conditioning agent is comprised in an amount is suitable and/or sufficient to generate; and/or
the energizing topical dosage form or the medical devices is/are suitable and/or configured to deliver, administer and/or apply conditioning energy to generate, preferably for a duration and/or an extend to generate:
   an accumulation of PBMCs within the skin, wherein it is to be understood that preferably any embodiment or definition thereof which is described under the outline note ‘As further regards step (A-0) for any of the embodiments as described herein’ or in any of the embodiments of the step (A) of the method as described herein is independently and mutatis mutandis applicable; and/or
   a vasodilation of the capillaries within the skin of the subject, wherein it is to be understood that preferably any embodiment or definition thereof which is described under the outline note ‘As further regards step (A-1) for any of the embodiments as described herein’ or in any of the embodiments of the step (A) of the method as described herein is independently and mutatis mutandis applicable; and/or
   an increased blood volume within the skin of the subject, wherein it is to be understood that preferably any embodiment or definition thereof which is described under the outline note ‘As further regards step (A-2) for any of the embodiments as described herein’ or in any of the embodiments of the step (A) of the method as described herein is independently and mutatis mutandis applicable; and/or
   an increased sO₂ and/or an increased rHb within the skin of the subject, wherein preferably the sO₂ is increased by 2 or more and/or the sO₂ is increased by 2%-points or more; and/or the rHb is increased by 2% or more and/or the rHb is increased by 2 AU (arbitrary units) or more, wherein it is to be understood that preferably any embodiment or definition thereof which is described under the outline note ‘As further regards step (A-3) for any of the embodiments as described herein’ or in any of the embodiments of the step (A) of the method as described herein is independently and mutatis mutandis applicable; and/or
   an increased temperature on the skin of the subject, wherein preferably the temperature is increased by 1% or more and/or the temperature is increased by 0.2° C. or more, wherein it is to be understood that preferably any embodiment or definition thereof which is described under the outline note ‘As further regards step (A-4) for any of the embodiments as described herein’ or in any of the embodiments of the step (A) of the method as described herein is independently and mutatis mutandis applicable; and/or
   a redness on the skin of the subject, wherein it is to be understood that preferably any embodiment or definition thereof which is described under the outline note ‘As further regards step (A-5) for any of the embodiments as described herein’ or in any of the embodiments of the step (A) of the method as described herein is independently and mutatis mutandis applicable.

Preferably, when one or more of the following conditions are fulfilled, namely when the pharmaceutical compositions, the topical dosage forms, the medical devices and/or the injectable dosage forms are:
administered or are used for an administration to the skin of the subject.
preferably used for an administration to the skin of the subject by topical application.
preferably used for an administration to the skin of the subject by injection.
preferably administered or used for an administration to a skin of a skin area an administration to a skin of a skin area.
preferably administered or used for an administration to the skin of the skin area, wherein the accumulation of PBMCs, vasodilation, increased blood volume, increased sO₂ and/or an increased rHb, increased temperature and/or redness is generated within the skin of the skin area.
preferably administered or used for an administration to the skin of the skin area by applying the pharmaceutical compositions,
the topical dosage forms and/or the injectable dosage forms to the skin of an application area. more preferably administered or used for an administration to the skin of the skin area by applying the pharmaceutical compositions, the topical dosage forms, the injectable dosage forms and/or the medical devices to the skin of the application area, wherein the increased concentration of the accumulation of PBMCs, vasodilation, increased blood volume, increased sO₂ and/or an increased rHb, increased temperature and/or redness is generated within the skin of the skin area.
are applied to the skin of the application area.
preferably applied to the skin of the application area, wherein the increased concentration of the accumulation of PBMCs, vasodilation, increased blood volume, increased sO₂ and/or an increased rHb, increased temperature and/or redness is generated within the skin of the skin area.
applied to the skin of the application area by topical application.
applied to the skin of the application area by injection.

Preferably, it is to be understood that any description and definition of the skin area as mentioned in any of the embodiments described herein, particularly of the method as described herein as mentioned in any of the embodiments of the method is independently and mutatis mutandis applicable to the skin area as mentioned in any of the embodiments of the pharmaceutical compositions, the injectable dosage forms, the topical dosage forms, the medical devices and/or the kits of parts described herein. More preferably, in any of the embodiments of the pharmaceutical compositions, the injectable dosage forms, the topical dosage forms and/or the medical devices described herein, the skin area is in case of the skin-conditioning agent, conditioning energy and/or heat the skin area [a] and/or the application area is the application area [a] as mentioned in any of the embodiments described herein particularly of step (A) of the method as described herein.

Preferably, in any of the embodiments of the pharmaceutical compositions, the injectable dosage forms, the topical dosage forms and/or the medical devices described herein, the skin area and/or the skin area [a] are independently and mutatis mutandis defined the same as the skin area and/or the skin area [a], respectively, as mentioned in any of the embodiments described herein, particularly of the method described herein. If not mentioned otherwise, it is to be understood that any embodiment or definition described herein in terms of a skin area and/or skin area [a] is independently and mutatis mutandis applicable to the skin area and/or skin area [a] in any of the embodiments of the pharmaceutical compositions, the injectable dosage forms, the topical dosage forms and/or the medical devices.

Preferably, in any of the embodiments of the pharmaceutical compositions, the injectable dosage forms, the topical dosage forms and/or the medical devices described herein, the application area and/or the application area [a] are independently and mutatis mutandis defined the same as the application area and/or the application area [a], respectively, as mentioned in any of the embodiments described herein, particularly of the method described herein. If not mentioned otherwise, it is to be understood that any embodiment or definition described herein in terms of an application area and/or application area [a] is independently and mutatis mutandis applicable to the application area and/or application area [a] in any of the embodiments of the pharmaceutical compositions, the injectable dosage forms, the topical dosage forms and/or the medical devices.

Particularly, it is to be understood that any embodiment or definition described herein in terms of the skin area, particularly the skin area [a], [b], [b₁] and/or [c], regarding the size, shape, overlapping and/or the area sum is independently and mutatis mutandis applicable to the skin area [a], [b], [b₁] and/or [c] as mentioned in any of the embodiments of pharmaceutical compositions, the topical dosage forms, the injectable dosage forms, the medical devices and/or the kits of parts described herein.

Particularly, it is to be understood that any embodiment or definition described herein in terms of the application area, particularly the application area [a], [b], [b₁] and/or [c], regarding the size, shape, overlapping and/or the area sum is independently and mutatis mutandis applicable to the application area [a], [b], [b₁] and/or [c] as mentioned in any of the embodiments of pharmaceutical compositions, the topical dosage forms, the injectable dosage forms, the medical devices and/or the kits of parts described herein.

Preferably, the pharmaceutical compositions, the injectable dosage forms, the topical dosage forms, the medical devices and/or the kits of parts are suitable and/or configured for intraepidermal, intradermal and/or subcutaneous, preferably for intradermal, administration, application and/or delivery of the substance(s) and/or skin-conditioning agent(s), particularly the substance(s) like the immunomodulatory substance(s), IFN-γ, IL-4, BDNF and/or IL-2.

Preferably, the pharmaceutical compositions, the injectable dosage forms, the topical dosage forms, the medical devices and/or the kits of parts are suitable and/or configured to administer, apply and/or deliver the substance(s), particularly the immunomodulatory substance(s), IFN-γ, IL-4, BDNF and/or IL-2:
into a sub-topical layer of the skin, more preferably, into the epidermis, dermis and/or subcutis, even more preferably the dermis, of the skin; and/or
6 mm or less to 0.1 mm or more, more preferably 4 mm or less to 0.3 mm or more, even more preferably 3 mm or less to 0.5 mm or more in skin thickness direction into the skin, when measured from the skin surface, preferably when measured perpendicular to the skin surface towards the bones.

Preferably, it is to be understood that any description and definition of the sub-topical layers as mentioned in any of the embodiments described herein, particularly of the method, more particularly of step (B), (B₁) and/or (C) of the method as described herein is independently and mutatis mutandis applicable to the sub-topical layer as mentioned in any of the embodiments of the pharmaceutical compositions, the injectable dosage forms, the topical dosage forms, the medical devices and/or the kits of parts described herein.

Preferably, the pharmaceutical compositions, the injectable dosage forms, the topical dosage forms, the medical devices and/or the kits of parts are suitable and/or configured for a delivery of the substance(s) and/or the skin-conditioning agent to the skin of the back, belly, buttocks, upper arm, forearm and/or upper leg more preferably the upper arm, forearm and/or upper leg.

Preferably, in the pharmaceutical compositions, the injectable dosage forms, the topical dosage forms, the medical devices and/or the kits of parts, the substances(s) and/or the agents(s) may be provided each spatially separated within the injectable dosage forms, or they may be provided as a mixture without spatial separation.

If no such spatial separation is present, this has the advantage to allow for an easier, simpler and less sophisticated production process to manufacture the pharmaceutical compositions, the injectable dosage forms, the topical dosage forms, the medical devices and/or the kits of parts.

The spatial separation may be accomplished by packaging or formulating the substance(s) and/or the skin-conditioning agent(s) into the particles within the pharmaceutical compositions, the injectable dosage forms, the topical dosage forms, the medical devices and/or the kits of parts. Furthermore, the spatial separation may be accomplished by providing the substance(s) and/or the skin-conditioning agent(s) in separated compartments or channels provided within the injectable dosage forms, the topical dosage forms and/or the medical devices. Moreover, the spatial separation may be accomplished by providing the substance(s) and/or the skin-conditioning agent(s) in separate pharmaceutical compositions, injectable dosage forms, topical dosage forms and/or the medical devices provided within the kits of parts.

A spatial separation has the advantage that is allows to store and hold the substance(s) and/or skin-conditioning agent(s) spatially separately within the pharmaceutical compositions, the injectable dosage forms, the topical dosage forms, the medical devices and/or the kits of parts. This may be for instance be appropriate in case the substance(s) and skin-conditioning agent(s) are not miscible, a mixture thereof is pharmaceutically not acceptable, the substances need different, incompatible formulation conditions to remain stable and active, or the substances are only approved by the authorities as individual formulations or compositions, but not as combination preparations.

Hence, preferably, the pharmaceutical compositions, the injectable dosage forms, the topical dosage forms, the medical devices and/or the kits of parts further comprise particles, particularly particles for drug delivery. More preferably the pharmaceutical compositions and/or the topical dosage form further comprise such particles.

The particles for drug delivery may be any particles known to the person skilled in the art which are suitable for delivery of drugs to a subject, particularly for delivery of the substance(s) and/or the skin-conditioning agent(s), particularly the immunomodulatory substance(s), IFN-γ, IL-4, BDNF and/or IL-2.

Preferably, the particles are suitable for transdermal administration, application and/or delivery, preferably for intraepidermal, intradermal and/or subcutaneous, more preferably for intradermal administration, application and/or delivery of the substance(s) and/or skin-conditioning agent(s), particularly the immunomodulatory substance(s), IFN-γ, IL-4, BDNF and/or IL-2.

Preferably the particles for drug delivery are suitable for an delivery of the substance(s) and/or skin-conditioning agent(s), more preferably the substance(s), into a sub-topical layer of the skin of the subject, more preferably into the epidermis, dermis and/or subcutis, even more preferably the dermis, and/or for an delivery 6 mm or less to 0.1 mm or more, more preferably 4 mm or less to 0.3 mm or more, even more preferably 2 mm or less to 0.5 mm or more into the skin of the subject in skin thickness direction when measured from the skin surface towards the bones.

The particles may be for instance nanoparticles, microparticles, lipid-based particles, liposomes, vesicles, micelles, silica magnetic nanoparticles, solid liquid nanoparticles, polymeric nanoparticles, solid nano-emulsions, nanoparticles suitable for coating with the substances(s) and/or skin-conditioning agent(s), dentrimers and/or carbon nanoparticles. The particles may be suitable for active or passive delivery, preferably they are suitable for passive delivery of the substance(s) and/or skin-conditioning agent(s).

Preferably, in the pharmaceutical compositions, the injectable dosage forms, the topical dosage forms, the medical devices and/or the kits of parts the substance(s) and/or skin-conditioning agent(s) are packed or formulated into the particles. Preferably the immunomodulatory substance(s), IFN-γ, IL-4, BDNF and/or IL-2 are packed or formulated into the particles.

The particles may contain a single or a combination of two or more selected from the substance(s) and/or skin-conditioning agent(s), preferably the immunomodulatory substance(s), IFN-γ, IL-4, BDNF, IL-2, and/or the skin-conditioning agent; more preferably the immunomodulatory substance(s), IFN-γ, IL-4, BDNF and/or IL-2; even more preferably the IFN-γ, IL-4, BDNF and/or IL-2. Each of the substance(s) and/or skin-conditioning agent(s) may be packed or formulated in separate particles or some or all of them may be packed or formulated in combinations of two of more thereof into particles. More preferably each substance and agent is packed or formulated in separate particles.

The pharmaceutical compositions, the injectable dosage forms, the topical dosage forms, the medical devices and/or the kits of parts may comprise any combination of such particles containing one or more of the substance(s) and/or the skin-conditioning agent(s). Alternatively, they may for instance comprise one or more of such particles packed with any one or more of the immunomodulatory substance(s) while the skin-conditioning agent may not be packed or formulated into the particles.

Preferably, the packaging and/or formulating of the substance(s) and/or the skin-conditioning agent(s) may be accomplished by any suitable method known to the person skilled in the art.

Preferably, the pharmaceutical compositions, the injectable dosage forms, the topical dosage forms, the medical devices and/or kits of parts as mentioned in any of the embodiments described herein further comprise, preferably in addition to the active ingredient(s), a pharmaceutically acceptable vehicle, diluent, adjuvant, excipient, carrier, additive and/or salt. These are preferably adapted for the respective form of administration like for instance a topical application or an injection. The use of these is well known in the art. Examples for the diluent, adjuvant or excipient are purified water, benzyl alcohol, cetyl alcohol, stearyl alcohol, polysorbate 80R, polysorbat 20, sorbitan stearate, glycerol, methyl-4-hydroxy benzoate, propyl-4-hydrobenzoate, isostearic acid, xanthan gum, D-mannitol, sodium succinate, succinic acid, sodium lauryl sulfate. Examples for the carrier, adjuvant, additive or salt are hydrophilic or lipophilic solvents, solubilizing agents, emulsifiers, stabilizers, fillers, buffering agents, solvents, isotonic agents, dispersion media, desiccants, antibacterial agents, antifungal agents, viscosity-adjusting agents, pH-adjusting agents, chelating agents, preservatives, and/or agents for providing a basis for topical application or agent for increasing skin-penetrating capacity.

The pharmaceutical compositions, the injectable dosage forms, the topical dosage forms, the medical devices and/or the kits of parts as mentioned in any of the embodiments described herein may be for use as a medicament. Hence, preferably, the pharmaceutical compositions, the injectable dosage forms, the topical dosage forms, the medical devices and/or the kits of parts are for use as a medicament.

Particularly, the pharmaceutical compositions, the injectable dosage forms, the topical dosage forms, the medical devices and/or the kits of parts may be for use in the treatment and/or prevention of diseases. Hence, more preferably, the pharmaceutical compositions, the injectable dosage forms, the topical dosage forms, the medical devices and/or the kits of parts are for use in the treatment and/or prevention of an inflammatory disease, immunological disease and/or autoimmunological disease in a subject.

If not mentioned otherwise, it is to be understood that any embodiment or definition described herein in terms of the inflammatory disease, immunological disease and/or autoimmunological disease in a subject is independently and mutatis mutandis applicable to the pharmaceutical compositions, dosage forms and the kits of parts in any of the embodiments thereof as described herein. More preferably, the inflammatory disease, immunological disease and/or autoimmunological disease to be treated and/or prevented comprises, preferably consists of, arthritis, synovitis, rheumatoid arthritis, polymyalgia rheumatica, Bechterew's disease, psoriatic arthritis, Hashimoto's disease, Basedow's disease and/or multiple sclerosis.

Furthermore, as already stated above, the present invention relates to a pharmaceutical composition (X), injectable dosage form (X), topical dosage form (X), medical device (X) and/or a kit of parts (X) for use and/or suitable for use in the method as mentioned in any of the embodiments described herein.

Preferably, the pharmaceutical composition (X) is any of the pharmaceutical compositions (I), (II), (III) and/or (IV) as mentioned in any of the embodiments described herein.

Preferably, the injectable dosage form (X) is any of the injectable dosage forms (I) and/or (II) as mentioned in any of the embodiments described herein.

Preferably, the topical dosage form (X) is the topical dosage form (I) and/or (II) as mentioned in any of the embodiments described herein.

Preferably, the medical device (X) is the medical device (100) as mentioned in any of the embodiments described herein.

Preferably, the kit of parts (X) is any of the kits of parts (I), (II), (III), (IV) and/or (V) as mentioned in any of the embodiments described herein.

Hence, the pharmaceutical compositions, the injectable dosage forms, the topical dosage forms, the medical devices and/or the kits of parts may be configured and/or suitable for use in the method as mentioned in any of the embodiments described herein. Therefore, preferably, the pharmaceutical compositions, the injectable dosage forms, the topical dosage forms, the medical devices and/or the kits of parts are suitable and/or configured for use in the method as mentioned in any of the embodiments described herein.

More preferably, the pharmaceutical compositions, the injectable dosage forms, the topical dosage forms, the medical devices and/or the kits of parts are suitable and/or configured for use in a method for treating and/or preventing an inflammatory disease, immunological disease and/or autoimmunological disease in a subject,
wherein the method comprises, preferably consists of, a step (A), wherein step (A) is selected from one or more of steps:
(A-0) generating an accumulation of PBMCs within the skin of the subject;
(A-1) generating a vasodilation of the capillaries within the skin of the subject;
(A-2) generating an increased blood volume within the skin of the subject;
(A-3) generating an increased sO₂ within the skin and/or an increased rHb within the skin of the subject;
(A-4) generating an increased temperature on the skin of the subject;
(A-5) generating a redness on the skin of the subject;
(A-6) administering conditioning energy to the skin of the subject;
(A-7) administering a skin-conditioning agent to the skin of the subject; and/or
(A-8) administering PBMCs into the skin of the subject,
and the method further comprises the step(s) of;
(B) administering the immunomodulatory substance(s) to the skin of said subject; and/or
(C) administering the immunomodulatory substance(s) to the skin of the subject, wherein the immunomodulatory substance(s) administered in step (C) is different from the immunomodulatory substance(s) administered in step (B).

The pharmaceutical compositions, the injectable dosage forms, the topical dosage forms, the medical devices and/or the kits of parts comprising the immunomodulatory substance(s), IFN-γ, IL-4, BDNF and/or IL-2 are particularly suitable for use in any one, two or all of steps (B), (B₁) and/or (C) of the method as described herein.

The pharmaceutical compositions, the injectable dosage forms, the topical dosage forms, the medical devices and/or the kits of parts which comprise the skin-conditioning agent and/or the energizing constituent are particularly suitable for use in step (A) of the method described herein. Furthermore, the topical dosage forms which are an energizing topical dosage form are particularly suitable for use in step (A) of the method as described herein.

Furthermore, the pharmaceutical compositions, the injectable dosage forms, the topical dosage forms, the medical devices and/or the kits of parts may be for use in the method as mentioned in any of the embodiments described herein.

Hence, more preferably, the pharmaceutical compositions, the injectable dosage forms, the topical dosage forms, the medical devices and/or the kit of parts are for use in the method as mentioned in any of the embodiments described herein.

Even more preferably, the pharmaceutical compositions, the injectable dosage forms, the topical dosage forms, the medical devices and/or the kits of parts are for use in a method for treating and/or preventing an inflammatory disease, immunological disease and/or autoimmunological disease in a subject,
wherein the method comprises, preferably consists of, a step (A), wherein step (A) is selected from one or more of steps:
(A-0) generating an accumulation of PBMCs within the skin of the subject;
(A-1) generating a vasodilation of the capillaries within the skin of the subject;
(A-2) generating an increased blood volume within the skin of the subject;
(A-3) generating an increased sO₂ within the skin and/or an increased rHb within the skin of the subject;
(A-4) generating an increased temperature on the skin of the subject;
(A-5) generating a redness on the skin of the subject;
(A-6) administering conditioning energy to the skin of the subject;
(A-7) administering a skin-conditioning agent to the skin of the subject; and/or
(A-8) administering PBMCs into the skin of the subject,
and the method further comprises the step(s) of;
(B) administering the immunomodulatory substance(s) to the skin of said subject; and/or
(C) administering the immunomodulatory substance(s) to the skin of the subject,
wherein the immunomodulatory substance(s) administered in step (C) is different from the immunomodulatory substance(s) administered in step (B).

The pharmaceutical compositions, the injectable dosage forms, the topical dosage forms, the medical devices and/or the kits of parts comprising the immunomodulatory substance(s), IFN-γ, IL-4, BDNF and/or IL-2 are particularly for use in any one, two or all of steps (B), (B₁) and/or (C) of the method as described herein.

The pharmaceutical compositions, the injectable dosage forms, the topical dosage forms, the medical devices and/or the kits of parts which comprise the skin-conditioning agent and/or the energizing constituent are, beside other steps, particularly for use in step (A) of the method described herein. Furthermore, the topical dosage forms which are an energizing topical dosage form are, beside other steps, particularly for use in step (A) of the method as described herein.

Preferably, the pharmaceutical compositions, injectable dosage forms, topical dosage forms, the medical devices and/or the kits of parts for use and/or suitable for use in the method are suitable or configured to accomplish, perform and/or effect one or more of steps (A), (B), (B₁) and/or (C) of the method as described herein, depending on which steps are applicable. Hence, in case the method comprises for instance steps (A) and (B) and/or (C) they are for instance suitable or configured to accomplish, perform and/or effect one or two of steps (A), (B) and/or (C).

It is more preferred, that the pharmaceutical compositions, injectable dosage forms, topical dosage forms, the medical devices and/or the kits of parts for use and/or suitable for use in the method are suitable or configured to accomplish, perform and/or effect all steps the method comprises. Hence, depending on which steps are applicable for the method, they are suitable or configured to accomplish, perform and/or effect all steps the method comprises. Hence, in case the method comprises steps (A), (B) and (C) they are suitable or configured to accomplish, perform and/or effect all of steps (A), (B) and (C). In case the method comprises steps (A), (B), (B₁) and (C) they are suitable or configured to accomplish, perform and/or effect all of steps (A), (B), (B₁) and (C). If the entire method, that is all steps of the method, is accomplished by the pharmaceutical compositions, injectable dosage forms, topical dosage forms, the medical devices and/or the kits of parts, this provides, amongst other, the advantages that no training of the user is demanded or inadequate training of the user is compensated when using them by performing the method. Furthermore, a correct execution of the method is ensured. An easy handling is provided which is particularly important for users having impaired manual skills as it is frequently the case for instance with rheumatological diseases. Moreover, a fear of self-application or self-injection errors can be taken away and a lack of treatment adherence can be mitigated or even overcome due to the simple and uncomplicated application of the method without a visit to the doctor.

Hence, particularly, the pharmaceutical compositions, the injectable dosage forms, the topical dosage forms, the medical devices and/or the kits of parts comprising the substance(s) like the immunomodulatory substance(s), IFN-γ, IL-4, BDNF and/or IL-2, and comprising the skin-conditioning agent, the skin-conditioning unit and/or the energizing constituent, or the topical dosage forms which are an energizing topical dosage form are suitable or configured to accomplish, perform and/or effect all steps the method comprises, that is step (A) of the method and one, two or all of steps (B), (B₁) and (C), depending on which steps the method comprises.

More particularly, in case the method comprises step (A) and one, two or all of steps (B), (B₁) and/or (C), the pharmaceutical compositions, injectable dosage forms, topical dosage forms, the medical devices and/or the kits of parts for use or suitable for use in the method and suitable or configured to accomplish, perform and/or effect all steps the method comprises are:
all embodiments of the pharmaceutical composition (I), and
all embodiments of the pharmaceutical compositions (II), (III), (IV), (X), all embodiments of the injectable dosage forms, all embodiments of the topical dosage forms, all embodiments of the medical devices and/or all embodiments of the kits of parts which are suitable and/or configured for generating the increased temperature on the skin, the increased sO₂ within the skin and/or the increased rHb within the skin,
even more particularly,
all embodiments of the pharmaceutical composition (I);
all embodiments of the pharmaceutical composition (II) which comprises a skin-conditioning agent;
all embodiments of the pharmaceutical compositions (III) which comprises a skin-conditioning agent;
all embodiments of the pharmaceutical compositions (IV) which comprise a skin-conditioning agent;
all embodiments of the injectable dosage form (I) which comprise a skin-conditioning agent; all embodiments of the injectable dosage form (II) which comprise a skin-conditioning agent;
all embodiments of the topical dosage form (I) which comprises a skin-conditioning agent and/or in which the topical dosage form (I) is an energizing topical dosage form;
all embodiments of the topical dosage form (II);
all embodiments of the medical device (100); and/or
all embodiments of the kits of parts (I), (III), (IV) and/or (V).

If not mentioned otherwise, it is to be understood that any of the embodiments or definitions described herein, particularly in any of the embodiments of the method and/or the medical use as described herein, is independently and mutatis mutandis applicable to any or all of the pharmaceutical compositions, injectable dosage forms, topical dosage forms, the medical devices and/or kit of parts as mentioned in any of the embodiments described herein.

Particularly, if not mentioned otherwise, it is to be understood that any embodiment or definition described herein for the:
the immunomodulatory substance(s); the cytokine-like acting substance(s); the interferon-like acting substance(s); the interleukin-like acting substance(s); the neurotrophin-like acting substance(s); the IFN-γ-like acting substance(s); the IL-2-like acting substance(s); the IL-4-like acting substance(s); the BDNF-like acting substance(s); the cytokine(s); the interferon(s); the interleukin(s); the neutrophin(s); the IFN-γ; the IL-2; the IL-4; the BDNF; the energizing means; the heating means; the conditioning energy; the heat; the skin-conditioning agent; the inflammatory disease, immunological disease and/or autoimmunological disease to be treated and/or prevented; the method; the increased concentration of the substance(s) and skin-conditioning agent(s); the skin area; the application area; and/or the subject,
particularly in terms of the method and/or the medical use, is independently and mutatis mutandis applicable to the pharmaceutical compositions (I), (II), (III), (IV) and/or (X), the injectable dosage form (I), (II) and/or (X), the topical dosage form (I), (II) and/or (X), the medical device (100) and/or (X), and/or the kit of parts (I), (II), (III), (IV), (V) and/or (X).

Furthermore, if not mentioned otherwise, it is to be understood that the expression “the pharmaceutical compositions, the injectable dosage forms, the topical dosage form, the medical devices and/or the kits of parts” refers independently to any or all of the pharmaceutical compositions (I), (II), (III), (IV) and/or (X), the injectable dosage form (I), (II) and/or (X), the topical dosage form (I), (II) and/or (X), the medical device (100) and/or (X) and/or the kits of parts (I), (II), (III), (IV), (V) and/or (X). Furthermore, if not mentioned otherwise, it is to be understood that any of the embodiments described herein in plural with reference to the pharmaceutical compositions, injectable dosage forms, topical dosage forms, the medical devices and/or the kits of parts refers independently to any of the pharmaceutical compositions (I), (II), (III), (IV) and/or (X), the injectable dosage form (I), (II) and/or (X), the topical dosage form (I), (II) and/or (X), the medical device (100) and/or (X) and/or the kits of parts (I), (II), (III), (IV), (V) and/or (X).

It is further to be understood that any of the embodiments mentioned under the outline note ‘As further regards independently any or all of the pharmaceutical compositions, the injectable dosage forms, the topical dosage forms, the medical devices and/or the kits of parts for any of the embodiments as described herein’ is independently applicable and/or combinable with any one of all of the embodiments stated under the outline notes:
“As further regards independently any or all of the pharmaceutical compositions, particularly the pharmaceutical compositions (I), (II), (III), (IV) and/or CO, for any of the embodiments as described herein”,
“As further regards independently any or all of the injectable dosage forms, particularly the injectable dosage form (I), (II) and/or CO for any of the embodiments as described herein”,
“As further regards independently any or all of the topical dosage forms, particularly the topical dosage form (I), (II) and/or topical dosage form (X) for any of the embodiments as described herein”,
“As further regards the medical device (100) for any of the embodiments as described herein”, and/or
“As further regards independently any or all of the kits of parts, particularly the kits of parts (I), (II), (III), (IV), (V) and/or CO, for any of the embodiments as described herein”.

The outline notes merely serve to structure the text and are not intended to have any delimiting or restrictive meaning beyond that. This applies to all outline notes throughout the entire description.

As further regards independently any or all of the pharmaceutical compositions, particularly the pharmaceutical compositions (I), (II), (III), (IV) and/or (X), for any of the embodiments as described herein

The pharmaceutical compositions may be administered and/or applied by any suitable route of administration to the skin.

Preferably, the pharmaceutical compositions are administered, applied and/or delivered by topically or by injection.

Preferably, the pharmaceutical compositions are suitable and/or prepared for a topical administration, application and/or delivery and/or they are suitable and/or prepared for an administration, application and/or delivery by injection.

Hence, the pharmaceutical compositions may be formulated or are in any form known to the person skilled in the art which is suitable for an administration, application and/or delivery of the substance(s) and/or skin-conditioning agent(s) to the skin. For example, the pharmaceutical compositions may independently be formulated or are independently in any form selected from an ointment, emulsion, solution, suspension, paste, gel, lotion, tincture, particulate material, powder, lyophilisate, liquid, dry preparation or solid preparations for application to the skin or for injection, slow-release formulation, wadding, tamponade, crème, balm, foam, gel, spray, stick, liquid plaster, spray plaster, transdermal patch, plaster, microneedle patch, patch, pad, wadding, padding, dressing, compress, bandage, wherein any of these are optionally of a multi-compartment type as mentioned in any of the embodiments described herein, injection solution, injection gel, preparation for intraepidermal, intradermal and/or subcutaneous injection, a dermatological preparation, preferably in the form of an application for external use or injection, cartridge for an injection pen or the medical device as described herein, carpule, vial, ampule, prefilled injection pen, prefilled syringe, prefilled multi-channel syringe, a medical device, wherein any of these are optionally of a multi-channel or multi-chamber type as mentioned in any of the embodiments described herein or any combination thereof, and/or in the form of the injectable dosage forms, the topical dosages forms and/or medical devices as mentioned in any of the embodiments described herein.

More preferably, the pharmaceutical compositions may preferably be provided in the form of:
the injectable dosage forms, preferably the injectable dosage forms (I) or (II), as mentioned in any of the embodiments described herein;
the topical dosage forms, preferably the topical dosage form (I) and/or (II), as mentioned in any of the embodiments described herein;
the medical devices, preferably the medical device (100), as mentioned in any of the embodiments described herein; and/or
the kits of parts, preferably the kits of parts (I), (II), (III), (IV) and/or (V), as mentioned in any of the embodiments described herein.

Preferably, the pharmaceutical compositions are suitable for a delivery, application or administration to the skin.

Preferably, the pharmaceutical compositions are suitable for an administration by topical application and/or by injection. Hence, preferably, the pharmaceutical compositions are a topical pharmaceutical composition or a pharmaceutical composition for injection.

Preferably, the topical pharmaceutical composition is for an application or administration on the skin.

Preferably, the pharmaceutical composition for injection is for an application or administration into the skin.

If not mentioned otherwise, it is to be understood that any embodiment or definition of for the pharmaceutical compositions described herein is independently and mutatis mutandis applicable to the topical pharmaceutical composition and the pharmaceutical composition for injection.

The topical pharmaceutical compositions may be formulated or are in any form suitable or prepared for a topical administration, application and/or delivery as it is known to the person skilled in the art. The topical pharmaceutical composition is for instance formulated into or in the form of a formulation selected from an ointment, emulsion, solution, suspension, paste, gel, lotion, tincture, particulate material, powder, lyophilisate, liquid, dry preparation or solid preparations for application to the skin, slow-release formulation, wadding, tamponade, crème, balm, foam, gel, spray, stick, liquid plaster, spray plaster, plaster for intradermal administration, time-release plaster transdermal patch, plaster, microneedle patch, patch, pad, wadding, padding, dressing, compress, bandage, wherein any of these are optionally of a multi-compartment type as mentioned in any of the embodiments described herein, a dermatological preparation, preferably in the form of an application for external use, and/or in the form of the topical dosages forms as mentioned in any of the embodiments described herein.

The pharmaceutical compositions for injection may be formulated or are in any form suitable or prepared for an administration, application and/or delivery by injection as it is known to the person skilled in the art. The pharmaceutical composition for injection is for instance formulated into or in the form of a formulation selected from an ointment, emulsion, solution, suspension, paste, gel, lotion, tincture, particulate material, liquid preparations for injection, slow-release formulation, crème, balm, foam, gel, a dermatological preparation, preferably in the form of an application for injection, cartridge for an injection pen or the medical device as described herein, carpule, vial, ampule, prefilled injection pen, prefilled syringe, prefilled multi-channel syringe, prefilled multi-channel syringe, a medical device, wherein any of these are optionally of a multi-channel or multi-chamber type as mentioned in any of the embodiments described herein or any combination thereof, and/or in the form of the injectable dosage forms as mentioned in any of the embodiments described herein.

In a still even further aspect, the present invention relates to the use of the pharmaceutical compositions as mentioned in any of the embodiments described herein for producing an article and/or a medical device.

Examples for the article may be a cartridge for an injection pen or the medical device as described herein, carpule, vial, ampule, prefilled injection pen, prefilled syringe, prefilled multi-channel syringe, wherein any of these are optionally of a multi-channel or multi-chamber type as mentioned in any of the embodiments described herein or any combination thereof, an injectable dosage form as mentioned in any of the embodiments described herein and/or an topical dosage form as mentioned in any of the embodiments described herein.

Examples for the medical device may be a prefilled injection pen, prefilled multi-channel injection pen, prefilled multi-chamber injection pen, prefilled injection pen, prefilled syringe, prefilled multi-channel syringe, prefilled multi-chamber syringe, an apparatus comprising a combination of an injection pen and an energizing means or a heating means and/or a medical device as mentioned in any of the embodiments described herein.

If not mentioned otherwise, it is to be understood that the expression “the pharmaceutical compositions” refers independently to any or all of the pharmaceutical compositions, particularly the pharmaceutical compositions (I), (II), (III), (IV) and/or (X), more particularly the pharmaceutical compositions (I), (II), (III) and/or (IV), as mentioned in any of the embodiments described herein. Furthermore, if not mentioned otherwise, it is to be understood that any of the embodiments described herein in plural with reference to the pharmaceutical compositions refers independently to any of the pharmaceutical compositions, particularly the pharmaceutical compositions (I), (II), (III), (IV) and/or (X), more particularly the pharmaceutical compositions (I), (II), (III) and/or (IV) as mentioned in any of the embodiments described herein.

It is further to be understood that any of the embodiments mentioned under the outline note ‘As further regards independently any or all of the pharmaceutical compositions, particularly the pharmaceutical compositions (I), (II), (III), (IV) and/or CO, for any of the embodiments as described herein’ is independently applicable and/or combinable with any of the embodiments of the pharmaceutical compositions, particularly as described under the outline note ‘As further regards independently any or all of the pharmaceutical compositions, the injectable dosage forms, the topical dosage forms, the medical devices and/or the kits of parts for any of the embodiments as described herein’.

As further regards independently any or all of the injectable dosage forms, particularly the injectable dosage form (I), (II) and/or (X) for any of the embodiments as described herein

Preferably, where applicable, the injectable dosage forms further comprise an immunomodulatory substance(s), wherein in case of the injectable dosage forms comprise:
the IFN-γ-like acting substance, the immunomodulatory substance(s) is different from the IFN-γ-like acting substance;
the IL-4-like acting substance, the immunomodulatory substance(s) is different from the IL-4-like acting substance;
the BDNF-like acting substance, the immunomodulatory substance(s) is different from the BDNF-like acting substance; and
the IL-2-like acting substance, the immunomodulatory substance(s) is different from the IL-2-like acting substance.

More preferably, where applicable, the injectable dosage forms further consist of an immunomodulatory substance(s) as further active ingredient, wherein in case of the injectable dosage forms consists of as active ingredient(s):
the IFN-γ-like acting substance, the immunomodulatory substance(s) is different from the IFN-γ-like acting substance;
the IL-4-like acting substance, the immunomodulatory substance(s) is different from the IL-4-like acting substance;
the BDNF-like acting substance, the immunomodulatory substance(s) is different from the BDNF-like acting substance; and
the IL-2-like acting substance, the immunomodulatory substance(s) is different from the IL-2-like acting substance.

More preferably, where applicable, the injectable dosage forms further comprise an immunomodulatory substance(s), wherein in case of the injectable dosage forms comprise:
IFN-γ, the immunomodulatory substance(s) is different from IFN-γ;
IL-4, the immunomodulatory substance(s) is different from IL-4;
BDNF, the immunomodulatory substance(s) is different from BDNF; and
IL-2, the immunomodulatory substance(s) is different from IL-2.

Even more preferably, where applicable, the injectable dosage forms further consist of an immunomodulatory substance(s) as further active ingredient, wherein in case of the injectable dosage forms consists of as active ingredient(s):
IFN-γ, the immunomodulatory substance(s) is different from IFN-γ;
IL-4, the immunomodulatory substance(s) is different from IL-4;
BDNF, the immunomodulatory substance(s) is different from BDNF; and
IL-2, the immunomodulatory substance(s) is different from IL-2.

Preferably, where applicable, the injectable dosage forms further comprise:
an IFN-γ-like acting substance(s) in a total amount equivalent to 30,000 IU IFN-γ or less and/or in a total amount equivalent in activity to 1,500 ng IFN-γ or less;
an IL-4-like acting substance(s) in a total amount equivalent in activity to 1,000 ng IL-4 or less;
an BDNF-like acting substance(s) in a total amount equivalent in activity to 500 ng BDNF or less; or
an IL-2-like acting substance(s) a total amount equivalent to 1,000 IU IL-2 or less and/or in a total amount equivalent in activity to 61 ng IL-2 or less.

More preferably, where applicable, the injectable dosage forms consist of as further active ingredient:
an IFN-γ-like acting substance(s) in a total amount equivalent to 30,000 IU IFN-γ or less and/or in a total amount equivalent in activity to 1,500 ng IFN-γ or less;
an IL-4-like acting substance(s) in a total amount equivalent in activity to 1,000 ng IL-4 or less;
an BDNF-like acting substance(s) in a total amount equivalent in activity to 500 ng BDNF or less; or
an IL-2-like acting substance(s) a total amount equivalent to 1,000 IU IL-2 or less and/or in a total amount equivalent in activity to 61 ng IL-2 or less.

More preferably, where applicable, the injectable dosage forms further comprise:
IFN-γ in a total amount of 30,000 IU or less and/or in a total amount of 1,500 ng or less;
IL-4 in a total amount of 1,000 ng or less;
BDNF in a total amount of 500 ng or less; or
IL-2 a total amount of 1,000 IU or less and/or in a total amount of 61 ng or less.

Even more preferably, where applicable, the injectable dosage forms further consist of as further active ingredient:
IFN-γ in a total amount of 30,000 IU or less and/or in a total amount of 1,500 ng or less;
IL-4 in a total amount of 1,000 ng or less;
BDNF in a total amount of 500 ng or less; or
IL-2 a total amount of 1,000 IU or less and/or in a total amount of 61 ng or less.

It is to be understood that the total amounts which are comprised in any of the embodiments of the topical dosage forms described herein below are independently and mutatis mutandis applicable to the total amounts which are comprised in any of the injectable dosage forms.

Preferably, the injectable dosage forms comprise one or more pharmaceutical compositions comprising, preferably consisting of, the IFN-γ, IL-4, BDNF, IL-2 and/or the immunomodulatory substance(s), and optionally comprising, preferably consisting of, the skin-conditioning agent, preferably as active ingredient(s).

More preferably, the injectable dosage forms comprise one or more pharmaceutical compositions comprising, preferably consisting of, the IFN-γ, IL-4, BDNF, IL-2 and/or the immunomodulatory substance(s), and optionally comprising, preferably consisting of, the skin-conditioning agent, preferably as active ingredient(s).

Preferably, such pharmaceutical compositions are any one or more of the pharmaceutical compositions as mentioned in any of the embodiments described herein. More preferably, the pharmaceutical compositions of the injectable dosage forms is any one or more of the pharmaceutical compositions as mentioned in any of the embodiments described herein.

Preferably, in the injectable dosage forms the immunomodulatory substance(s), IFN-γ-like acting substance(s), IL-4-like acting substance(s), BDNF-like acting substance(s), IL-2 like acting substance(s), IFN-γ, IL-4, BDNF, IL-2 and/or the skin-conditioning agent is in the form of a pharmaceutical composition, more preferably in the form of any of the pharmaceutical compositions as mentioned in any of the embodiments described herein.

Preferably, the injectable dosage forms further comprise a pharmaceutically acceptable vehicle, diluent, adjuvant, excipient, carrier, additive and/or salt as mentioned in any of the embodiments described herein.

Preferably, in the injectable dosage forms, the comprised components, like the pharmaceutical compositions, the substances(s), the agents(s) and/or the pharmaceutically acceptable vehicles, diluents, adjuvants, excipients, carriers, additives and/or salts, may be provided each spatially separated within the injectable dosage forms, e.g. in separate compartments, chambers, channels provided within the injectable dosage forms, or by formulating some or all of the comprised components, particularly the pharmaceutical compositions, substances(s) and/or the agents(s), into particles, or the comprised components may be provided as a mixture without spatial separation, or any combination thereof. The particles are preferably any of the particles as mentioned in any of the embodiments described herein.

Hence, the injectable dosage forms may comprise without spatial separation a pharmaceutical composition comprising, preferably consisting of, the IFN-γ, IL-4, BDNF, IL-2, the immunomodulatory substance(s) and/or the skin-conditioning agent, preferably as active ingredient(s).

Preferably, in the injectable dosage forms, at least one of the comprised components may be provided spatially separated within the injectable dosage forms, e.g. in separate compartments, chambers and/or channels provided within the injectable dosage forms.

More preferably, the injectable dosage forms comprise:
(i) spatially separated
   one or more pharmaceutical compositions comprising, preferably consisting of as active ingredient(s), the IFN-γ, IL-4, BDNF and/or IL-2, and/or
   optionally a further pharmaceutical composition comprising, preferably consisting of as further active ingredient the immunomodulatory substance(s), and/or
   optionally a further pharmaceutical composition comprising, preferably consisting of as further active ingredient the skin-conditioning agent, and/or
   optionally one or more of the pharmaceutically acceptable vehicles, diluents, adjuvants, excipients, carriers, additives and/or salts,
or
(ii) spatially separated,
   one or more of the IFN-γ, IL-4, BDNF and/or IL-2, preferably consisting thereof as active ingredient(s), and/or
   optionally a further immunomodulatory substance(s), preferably consisting thereof as further active ingredient, and/or
   optionally the skin-conditioning agent, preferably consisting thereof as further active ingredient, and
   one or more of the pharmaceutically acceptable vehicles, diluents, adjuvants, excipients, carriers, additives and/or salts,
or
(iii) spatially separated
   one or more pharmaceutical compositions comprising, preferably consisting of as active ingredient(s), the IFN-γ, IL-4, BDNF and/or IL-2, optionally the immunomodulatory substance(s) and/or optionally the skin-conditioning agent, and
   optionally one or more pharmaceutically acceptable vehicles, diluents, adjuvants, excipients, carriers, additives and/or salts,
or
(iv) spatially separated,
   one or more of the substances(s) like the IFN-γ, IL-4, BDNF, IL-2, the immunomodulatory substance(s) and/or the skin-conditioning agent, preferably consisting thereof as active ingredient(s), and
   one or more pharmaceutically acceptable vehicles, diluents, adjuvants, excipients, carriers, additives and/or salts,
or
(v) any combination thereof,
or the injectable dosage forms comprise any of (i) to (v) in any combination with,
   a pharmaceutical composition without spatial separation, wherein the pharmaceutical composition comprises, preferably consists of, the IFN-γ, IL-4, BDNF, IL-2, the immunomodulatory substance(s) and/or the skin-conditioning agent, preferably as active ingredient(s).

The special separation stated under items (ii) and (iv) are particularly useful if any or all of the substance(s) and/or skin-conditioning agent(s) are provided as a powder, lyophilisate, dry preparation or solid preparation.

Preferably, the injectable dosage forms are an article.

Preferably, the article has a hollow body comprising at least one compartment, chamber or channel for holding the comprised components, wherein the hollow body may completely enclose the compartment, chamber or channel like a glass ampule that must be broken open to be opened, or the hollow body may have at least one opening closed by a closure like a lid, rubber stopper, cap, plunger like a syringe plunger and/or cannula like an injection cannula etc.

More preferably, the injectable dosage forms and/or the article is a cartridge for an injection pen or the medical device as described herein, a carpule, a vial, an ampule, an injection pen, a syringe, a multi-channel syringe and/or a medical device. Optionally the injectable dosage forms and/or the article particularly the cartridge for an injection pen or the medical device as described herein, a carpule, a vial, an ampule, an injection pen, a syringe, a multi-channel syringe and/or a medical device, is of a multi-compartment type.

Preferably, the medical device is the medical device as mentioned in any of the embodiments described herein.

Preferably, within the injectable dosage forms, the comprised components are spatially separated within the injectable dosage forms.

The comprised components may be for instance the pharmaceutical compositions, substance(s), skin-conditioning agent(s), pharmaceutically acceptable vehicles, diluents, adjuvants, excipients, carriers, additives and/or salts, The spatial separation may be provided by separate compartments, channels or chambers provided within the injectable dosage forms, for instance when the injectable dosage form is of the multi-channel or multi-chamber type or any combination thereof, or by packaging or formulating one or more of the comprised components into the particles, or by any combination thereof. The particles are preferably any particles as mentioned in any of the embodiments described herein.

A multi-channel or multi-chamber type and particularly injectable dosage forms of the multi-channel or multi-chamber type may have two or more channels and/or two or more chambers for spatial separation of the comprised components. They allow for a mixing, reconstituting or dissolving of the comprised components previous to the delivery to the subject. Furthermore, they allow for a parallel and simultaneous or essentially simultaneous delivery of the comprised components to the subject, with or without previous mixing, reconstituting or dissolving them. Examples for a multi-chamber type and particularly for an injectable dosage form of a multi-chamber type are any known in the art like a multi- chamber injection pen, a multi-chamber carpule like a dual-chamber carpule, a multi-compartment vial, a multi-chamber ampule or a multi-chamber syringe, a vial having lid, wherein vial and lid have chambers separated by a separation and the separation is mechanically destroy by activating the lid, allowing the components contained in the lid to be mixed, reconstituted or solved in the components contained in the vial. Examples for a multi-channel type and particularly for an injectable dosage form of a multi-channel type are any known in the art like a multi-channel injection pen, a multi-channel carpule, a multi-channel vial, a multi-channel ampule or a multi-channel syringe like a dual-channel syringe having two injection cannulas (no previous mixing) or a single injection cannula (provides previous mixing).

By using injectable dosage forms providing spatial separation, like injectable dosage forms of the multi-channel or multi-chamber type, the comprised components may be stored spatially separately within the injectable dosage forms. The delivery or administration of the comprised components may then be accomplished without previous mixing, reconstituting or dissolving them. This for instance possible by means of a multi-channel syringe, wherein each channel is functionally connected with a separate injection cannula, allowing for separate delivery of the comprised components using a single article or medical device. Alterantively, the delivery or administration of the comprised components may be accomplished including previous mixing, reconstituting or dissolving. The latter is for instance possible by means of e.g. a multi-channel syringe, wherein the individual channels are functionally connected to a common cannula, or a multi-chamber cartridge or carpule. This may be for instance appropriate in case the comprised components are not miscible; a mixture thereof is pharmaceutically not acceptable; the individual comprised components, particularly the substance(s) and/or skin-conditioning agent(s), need different, incompatible formulation conditions to remain stable and active; the substance(s) and/or skin-conditioning agent(s), particularly the substance(s), are only stable in dry form like a lyophilisate and require reconstitution in e.g. a pharmaceutically acceptable diluent directly before delivery to the patient for instance by injection; or the comprised components, particularly the substance(s) and/or skin-conditioning agent(s), are only approved by the authorities in individual formulations or compositions, but not as combination preparations.

However, if no spatial separation is present, this has the advantage that the production processes to manufacture the injectable dosage forms and/or the article are easier, simpler and less sophisticated.

Preferably, the injectable dosage forms are filled or prefilled with one or more of the pharmaceutical compositions, the IFN-γ, IL-4, BDNF, IL-2, the immunomodulatory substance(s) and/or the skin-conditioning agent and optionally the pharmaceutically acceptable vehicles, diluents, adjuvants, excipients, carriers, additives and/or salts. More preferably, the injectable dosage forms are exclusively filled or prefilled with one or more of the pharmaceutical compositions, the IFN-γ, IL-4, BDNF, IL-2, the immunomodulatory substance(s) and/or the skin-conditioning agent as active ingredient(s).

More preferably, the injectable dosage forms are filled or prefilled with one or more of the pharmaceutical compositions as mentioned in any of the embodiments described herein, preferably as active ingredient(s). More preferably, the injectable dosage forms are exclusively filled or prefilled with one or more of the pharmaceutical compositions as active ingredient(s).

The injectable dosage forms may be for single or repeated use, preferably for single use.

Moreover, the present invention relates to the use of the injectable dosage forms as mentioned in any of the embodiments described herein for producing a medical device.

Examples for the medical device may be an injection pen, an apparatus comprising a combination of an injection pen and an energizing means, an apparative energizing means, a heating means, an apparative heating means and/or a medical device as mentioned in any of the embodiments described herein, wherein any of these are optionally of a multi-channel or multi-chamber type or any combination thereof.

If not mentioned otherwise, it is to be understood that the expression “the injectable dosage forms” refers to any or all of the injectable dosage forms, particularly the injectable dosage form (I), (II) and/or (X), more particularly the injectable dosage form (I) and/or (II), as mentioned in any of the embodiments described herein. Furthermore, if not mentioned otherwise, it is to be understood that any of the embodiments described herein in plural with reference to the injectable dosage forms refers independently to any of the injectable dosage forms, particularly the injectable dosage forms, particularly the injectable dosage form (I), (II) and/or (X), more particularly the injectable dosage form (I) and/or (II), as mentioned in any of the embodiments described herein.

It is further to be understood that any of the embodiments mentioned under the outline note ‘As further regards independently any or all of the injectable dosage forms, particularly the injectable dosage form (I), (II) and/or (X), for any of the embodiments as described herein’ is independently applicable and/or combinable with any of the embodiments of the injectable dosage forms, particularly as described under the outline note ‘As further regards independently any or all of the pharmaceutical compositions, the injectable dosage forms, the topical dosage forms, the medical devices and/or the kits of parts for any of the embodiments as described herein’.

As further regards independently any or all of the topical dosage forms, particularly the topical dosage form (I), (II) and/or topical dosage form (X) for any of the embodiments as described herein

Preferably, where applicable, the topical dosage forms further comprise an immunomodulatory substance(s), wherein in case of the topical dosage forms comprise:
the IFN-γ-like acting substance, the immunomodulatory substance(s) is different from the IFN-γ-like acting substance;
the IL-4-like acting substance, the immunomodulatory substance(s) is different from the IL-4-like acting substance;
the BDNF-like acting substance, the immunomodulatory substance(s) is different from the BDNF-like acting substance; and
the IL-2-like acting substance, the immunomodulatory substance(s) is different from the IL-2-like acting substance.

More preferably, where applicable, the topical dosage forms further consist of an immunomodulatory substance(s) as further active ingredient, wherein in case of the topical dosage forms consists of as active ingredient(s):
the IFN-γ-like acting substance, the immunomodulatory substance(s) is different from the IFN-γ-like acting substance;
the IL-4-like acting substance, the immunomodulatory substance(s) is different from the IL-4-like acting substance;
the BDNF-like acting substance, the immunomodulatory substance(s) is different from the BDNF-like acting substance; and
the IL-2-like acting substance, the immunomodulatory substance(s) is different from the IL-2-like acting substance.

More preferably, where applicable, the topical dosage forms further comprise an immunomodulatory substance(s), wherein in case of the topical dosage forms comprise:
IFN-γ, the immunomodulatory substance(s) is different from IFN-γ;
IL-4, the immunomodulatory substance(s) is different from IL-4;
BDNF, the immunomodulatory substance(s) is different from BDNF; and
IL-2, the immunomodulatory substance(s) is different from IL-2.

Even more preferably, where applicable, the topical dosage forms further consist of an immunomodulatory substance(s) as further active ingredient, wherein in case of the topical dosage forms consists of as active ingredient(s):
IFN-γ, the immunomodulatory substance(s) is different from IFN-γ;
IL-4, the immunomodulatory substance(s) is different from IL-4;
BDNF, the immunomodulatory substance(s) is different from BDNF; and
IL-2, the immunomodulatory substance(s) is different from IL-2.

Preferably, where applicable, the topical dosage forms comprise or further comprise:
IFN-γ-like acting substance(s) in a total amount equivalent to 30,000 IU IFN-γ or less and/or in a total amount equivalent in activity to 1,500 ng IFN-γ or less;
IL-4-like acting substance(s) in a total amount equivalent in activity to 1,000 ng IL-4 or less;
BDNF-like acting substance(s) in a total amount equivalent in activity to 500 ng BDNF or less; or
IL-2-like acting substance(s) a total amount equivalent to 1,000 IU IL-2 or less and/or in a total amount equivalent in activity to 61 ng IL-2 or less.

More preferably, where applicable, the topical dosage forms consist of as active ingredient or as further active ingredient:
IFN-γ-like acting substance(s) in a total amount equivalent to 30,000 IU IFN-γ or less and/or in a total amount equivalent in activity to 1,500 ng IFN-γ or less;
IL-4-like acting substance(s) in a total amount equivalent in activity to 1,000 ng IL-4 or less;
BDNF-like acting substance(s) in a total amount equivalent in activity to 500 ng BDNF or less; or
IL-2-like acting substance(s) a total amount equivalent to 1,000 IU IL-2 or less and/or in a total amount equivalent in activity to 61 ng IL-2 or less.

More preferably, where applicable, the topical dosage forms comprise or further comprise:
IFN-γ in a total amount of 30,000 IU or less and/or in a total amount of 1,500 ng or less;
IL-4 in a total amount of 1,000 ng or less;
BDNF in a total amount of 500 ng or less; or
IL-2 a total amount of 1,000 IU or less and/or in a total amount of 61 ng or less.

Even more preferably, where applicable, the topical dosage forms further consist of as active ingredient or as further active ingredient:
IFN-γ in a total amount of 30,000 IU or less and/or in a total amount of 1,500 ng or less;
IL-4 in a total amount of 1,000 ng or less;
BDNF in a total amount of 500 ng or less; or
IL-2 a total amount of 1,000 IU or less and/or in a total amount of 61 ng or less.

If not mentioned otherwise, it is to be understood that any embodiment or definition described herein in terms of the total amount of IFN-γ, IL-4, BDNF and/or IL-2 in any of the embodiments of the method described herein, more particularly of step (B), (B₁) and/or (C) of the method and which amounts are indicated in IU (international units) or ng (nanogram) is independently and mutatis mutandis applicable to the total amounts which are comprised in any of the topical dosage forms described herein and which are indicated in IU or ng, respectively.

Preferably, the topical dosage forms comprise IFN-γ-like acting IFN-γ substance(s) in the following IFN-γ total amounts:
Preferably, IFN-γ-like acting IFN-γ substance(s) in a total amount equivalent to 30,000 IU IFN-γ or less, more preferably 15,000 IU IFN-γ or less, even more preferably 10,000 IU IFN-γ or less, still more preferably 5,000 IU IFN-γ or less, still even more preferably 2,500 IU IFN-γ or less, further preferably 1,500 IU IFN-γ or less, still further preferably 1,000 IU IFN-γ or less, even further preferably 300 IU IFN-γ or less. There is no lower limit for the amount as long IFN-γ as the beneficial effects can be achieved, however, usually, a lower limit is a total amount equivalent to 2 IU IFN-γ or more, more preferably 10 IU IFN-γ or more, even more preferably 20 IU IFN-γ or more, still more preferably 30 IU IFN-γ or more and still even more preferably 50 IU IFN-γ or more. More preferably, IFN-γ-like acting IFN-γ substance(s) is comprised in a total amount in the rang IFN-γe of equivalent to 30,000 IU IFN-γ or less and 2 IU IFN-γ or more, even more preferably 15,000 IU IFN-γ or less and 2 IU IFN-γ or more, still more preferably 10,000 IU IFN-γ or less and 10 IU IFN-γ or more, still even more preferably 5,000 IU IFN-γ or less and 10 IU IFN-γ or more, further preferably 2,500 IU IFN-γ or less and 20 IU IFN-γ or more, even further preferably 1,500 IU IFN-γ or less and IU IFN-γ or more, still further preferably 1,000 IU IFN-γ or less and 50 IU IFN-γ or more, still even further preferably 300 IU IFN-γ or less and 50 IU IFN-γ or more.
Preferably, IFN-γ-like acting IFN-γ substance(s) a total amount equivalent in activity to 1,500 ng IFN-γ or less, more preferably 750 ng IFN-γ or less, even more preferably 500 ng IFN-γ or less, still more preferably 250 ng IFN-γ or less, still even more preferably 125 ng IFN-γ or less, further preferably 75 ng IFN-γ or less, still further preferably 50 ng IFN-γ or less, even further preferably 15 ng IFN-γ or less. There is no lower limit for the amount, preferably the total amount, as long IFN-γ as the beneficial effects can be achieved, however, usually, a lower limit is a total amount equivalent in activity to 0.1 ng IFN-γ or more, more preferably 0.5 ng IFN-γ or more, even more preferably 1 ng IFN-γ or more and still even more preferably 2 ng IFN-γ or more. More preferably, IFN-γ-like acting IFN-γ substance(s) is comprised in a total amount in the rang IFN-γe of equivalent in activity to 1,500 ng IFN-γ or less and 0.1 ng IFN-γ or more, even more preferably 750 ng IFN-γ or less and 0.1 ng IFN-γ or more, still more preferably 500 ng IFN-γ or less and 0.1 ng IFN-γ or more, still even more preferably 250 ng IFN-γ or less and 0.5 ng IFN-γ or more, further preferably 125 ng IFN-γ or less and 0.5 ng IFN-γ or more, even further preferably 75 ng IFN-γ or less and 1 ng IFN-γ or more, still further preferably 50 ng IFN-γ or less and 1 ng IFN-γ or more, still even further preferably 15 ng IFN-γ or less and 2 ng IFN-γ or more.
The same amounts apply to any derivative, fragment, protein-analogue, peptide, protein-variant, biopharmaceutical, inductor, precursor, prodrug, mutein, co-drug, propeptide and/or any pharmaceutically acceptable salt of the IFN-γ.

Preferably, the topical dosage forms comprise IL-2-like acting substance(s) in the following total amounts:
Preferably, IL-2-like acting IL-2 substance(s) a total amount equivalent to 1,000 IU IL-2 or less, more preferably 500 IU IL-2 or less, even more preferably 400 IU IL-2 or less, still more preferably 300 IU IL-2 or less, still even more preferably 250 IU IL-2 or less. There is no lower limit for the amount, preferably the total amount, as long IL-2 as the beneficial effects can be achieved, however, usually, a lower limit is a total amount equivalent to 25 IU IL-2 or more, more preferably 50 IU IL-2 or more, even more preferably 100 IU IL-2 or more. More preferably, the IL-2-like acting IL-2 substance(s) is comprised in a total amount in the rang IL-2e of equivalent to 1,000 IU IL-2 or less and 25 IU IL-2 or more, even more preferably 500 IU IL-2 or less and 25 IU IL-2 or more, still more preferably 400 IU IL-2 or less and 50 IU IL-2 or more, still even more preferably 300 IU IL-2 or less and 50 IU IL-2 or more, further preferably 250 IU IL-2 or less and 100 IU IL-2 or more.
Preferably, IL-2-like acting IL-2 substance(s) a total amount equivalent in activity to 61 ng IL-2 or less, more preferably 30.5 ng IL-2 or less, even more preferably 24.5 ng IL-2 or less, still more preferably 18.3 ng IL-2 or less, still even more preferably 12.2 ng IL-2 or less. There is no lower limit for the amount, preferably the total amount, as long IL-2 as the beneficial effects can be achieved, however, usually, a lower limit is a total amount equivalent in activity to 2 ng IL-2 or more, more preferably 3 ng IL-2 or more, even more preferably 6.1 ng IL-2 or more. More preferably, IL-2-like acting IL-2 substance(s) is comprised in a total amount in the rang IL-2e of equivalent in activity to 61 ng IL-2 or less and 2 ng IL-2 or more, even more preferably 30.5 ng IL-2 or less and 2 ng IL-2 or more, still more preferably 24.5 ng IL-2 or less and 3 ng IL-2 or more, still even more preferably 28.3 ng IL-2 or less and 3 ng IL-2 or more, still even more preferably 12.1 ng IL-2 or less and 6.1 ng IL-2 or more.
The same amounts apply to any derivative, fragment, protein-analogue, peptide, protein-variant, biopharmaceutical, inductor, precursor, prodrug, mutein, co-drug, propeptide and/or any pharmaceutically acceptable salt of the IL-2.

Preferably, the topical dosage forms comprise IL-4-like acting substance(s) in the following total amounts:
Preferably, the IL-4-like acting IL-4 substance(s) a total amount equivalent in activity to 1,000 ng IL-4 or less, more preferably 500 ng IL-4 or less, even more preferably 80 ng IL-4 or less, still more preferably 50 ng IL-4 or less, still even more preferably ng IL-4 or less, still even more preferably 15 ng IL-4 or less. There is no lower limit for the amount, preferably the total amount, as long IL-4 as the beneficial effects can be achieved, however, usually, a lower limit is an total amount equivalent in activity to 0.1 ng IL-4 or more, more preferably 0.5 ng IL-4 or more, even more preferably 1 ng IL-4 or more and still even more preferably 2 ng IL-4 or more. More preferably, IL-4-like acting IL-4 substance(s) is compried in a total amount in the rang IL-4e of equivalent in activity to 1,000 ng IL-4 or less and 0.1 ng IL-4 or more, even more preferably 500 ng IL-4 or less and 0.1 ng IL-4 or more, still more preferably 80 ng IL-4 or less and 0.5 ng IL-4 or more, still even more preferably 50 ng IL-4 or less and 0.5 ng IL-4 or more, further preferably 20 ng IL-4 or less and 1 ng IL-4 or more, even further preferably 15 ng IL-4 or less and 2 ng IL-4 or more.
The same amounts apply to any derivative, fragment, protein-analogue, peptide, protein-variant, biopharmaceutical, inductor, precursor, prodrug, mutein, co-drug, propeptide and/or any pharmaceutically acceptable salt of the IL-4.

Preferably, the topical dosage forms comprise BDNF-like acting substance(s) in the following total amounts:
Preferably, BDNF-like acting BDNF substance(s) a total amount equivalent in activity to 500 ng BDNF or less, more preferably 100 ng BDNF or less, even more preferably 50 ng BDNF or less, still more preferably 25 ng BDNF or less, still even more preferably 10 ng BDNF or less, further preferably 5 ng BDNF or less. There is no lower limit for the amount, preferably the total amount, as long BDNF as the beneficial effects can be achieved, however, usually, a lower limit is a total amount equivalent in activity to 0.005 ng BDNF or more, more preferably 0.01 ng BDNF or more, even more preferably 0.05 ng BDNF or more, still even more preferably 0.1 ng BDNF or more, further preferably 0.2 ng BDNF or more. More preferably, BDNF-like acting BDNF substance(s) is comprised a total amount in the rang BDNFe of 500 ng BDNF or less and 0.005 ng BDNF or more, even more preferably 100 ng BDNF or less and 0.01 ng BDNF or more, still more preferably 50 ng BDNF or less and 0.05 ng BDNF or more, still even more preferably 25 ng BDNF or less and 0.05 ng BDNF or more, further preferably 10 ng BDNF or less and 0.1 ng BDNF or more, even further preferably 5 ng BDNF or less and 0.2 ng BDNF or more.
The same amounts apply to any derivative, fragment, protein-analogue, peptide, protein-variant, biopharmaceutical, inductor, precursor, prodrug, mutein, co-drug, propeptide and/or any pharmaceutically acceptable salt of the BDNF.

Preferably, the topical dosage forms comprise IFN-γ in the following total amounts:
Preferably, IFN-γ a total amount of 30,000 IU or less, more preferably 15,000 IU or less, even more preferably 10,000 IU or less, still more preferably 5,000 IU or less, still even more preferably 2,500 IU or less, further preferably 1,500 IU or less, still further preferably 1,000 IU or less, even further preferably 300 IU or less. There is no lower limit for the amount as long as the beneficial effects can be achieved, however, usually, a lower limit is 2 IU or more, more preferably 10 IU or more, even more preferably IU or more, still more preferably 30 IU or more and still even more preferably 50 IU or more. More preferably, IFN-γ is comprised in a total amount in the range of 30,000 IU or less and 2 IU or more, even more preferably 15,000 IU or less and 2 IU or more, still more preferably 10,000 IU or less and 10 IU or more, still even more preferably 5,000 IU or less and 10 IU or more, further preferably 2,500 IU or less and 20 IU or more, even further preferably 1,500 IU or less and 30 IU or more, still further preferably 1,000 IU or less and 50 IU or more, still even further preferably 300 IU or less and 50 IU or more.
Preferably, IFN-γ a total amount of 1,500 ng or less, more preferably 750 ng or less, even more preferably 500 ng or less, still more preferably 250 ng or less, still even more preferably 125 ng or less, further preferably 75 ng or less, still further preferably 50 ng or less, even further preferably 15 ng or less. There is no lower limit for the amount, preferably the total amount, as long as the beneficial effects can be achieved, however, usually, a lower limit is 0.1 ng or more, more preferably 0.5 ng or more, even more preferably 1 ng or more and still even more preferably 2 ng or more. More preferably, IFN-γ is comprised a total amount in the range of 1,500 ng or less and 0.1 ng or more, even more preferably 750 ng or less and 0.1 ng or more, still more preferably 500 ng or less and 0.1 ng or more, still even more preferably 250 ng or less and 0.5 ng or more, further preferably 125 ng or less and 0.5 ng or more, even further preferably 75 ng or less and 1 ng or more, still further preferably 50 ng or less and 1 ng or more, still even further preferably 15 ng or less and 2 ng or more.

Preferably, the topical dosage forms comprise IL-2 in the following total amounts:
Preferably, IL-2 a total amount of 1,000 IU or less, more preferably 500 IU or less, even more preferably 400 IU or less, still more preferably 300 IU or less, still even more preferably 250 IU or less. There is no lower limit for the amount, preferably the total amount, as long as the beneficial effects can be achieved, however, usually, a lower limit is 25 IU or more, more preferably 50 IU or more, even more preferably 100 IU or more. More preferably, IL-2 is comprised in a total amount in the range of 1,000 IU or less and 25 IU or more, even more preferably 500 IU or less and 25 IU or more, still more preferably 400 IU or less and 50 IU or more, still even more preferably 300 IU or less and 50 IU or more, further preferably 250 IU or less and 100 IU or more. Preferably, IL-2 a total amount of 61 ng or less, more preferably 30.5 ng or less, even more preferably 24.5 ng or less, still more preferably 18.3 ng or less, still even more preferably 12.2 ng or less. There is no lower limit for the amount, preferably the total amount, as long as the beneficial effects can be achieved, however, usually, a lower limit is 2 ng or more, more preferably 3 ng or more, even more preferably 6.1 ng or more. More preferably, IL-2 is comprised in a total amount, in the range of 61 ng or less and 2 ng or more, even more preferably 30.5 ng or less and 2 ng or more, still more preferably 24.5 ng or less and 3 ng or more, still even more preferably 28.3 ng or less and 3 ng or more, still even more preferably 12.1 ng or less and 6.1 ng or more.

Preferably, the topical dosage forms comprise IL-4 in the following total amounts:
Preferably, the IL-4 a total amount of 1,000 ng or less, more preferably 500 ng or less, even more preferably 80 ng or less, still more preferably 50 ng or less, still even more preferably 20 ng or less, still even more preferably 15 ng or less. There is no lower limit for the amount, preferably the total amount, as long as the beneficial effects can be achieved, however, usually, a lower limit is 0.1 ng or more, more preferably 0.5 ng or more, even more preferably 1 ng or more and still even more preferably 2 ng or more. More preferably, IL-4 is comprised in a total amount in the range of 1,000 ng or less and 0.1 ng or more, even more preferably 500 ng or less and 0.1 ng or more, still more preferably 80 ng or less and 0.5 ng or more, still even more preferably 50 ng or less and 0.5 ng or more, further preferably 20 ng or less and 1 ng or more, even further preferably 15 ng or less and 2 ng or more.

Preferably, the topical dosage forms comprise BDNF in the following total amounts:
Preferably, BDNF a total amount of 500 ng or less, more preferably 100 ng or less, even more preferably 50 ng or less, still more preferably 25 ng or less, still even more preferably 10 ng or less, further preferably 5 ng or less. There is no lower limit for the amount, preferably the total amount, as long as the beneficial effects can be achieved, however, usually, a lower limit is 0.005 ng or more, more preferably 0.01 ng or more, even more preferably 0.05 ng or more, still even more preferably 0.1 ng or more, further preferably 0.2 ng or more. More preferably, BDNF is comprised in a total amount in the range of 500 ng or less and 0.005 ng or more, even more preferably 100 ng or less and 0.01 ng or more, still more preferably 50 ng or less and 0.05 ng or more, still even more preferably 25 ng or less and 0.05 ng or more, further preferably 10 ng or less and 0.1 ng or more, even further preferably 5 ng or less and 0.2 ng or more.

Preferably, the topical dosage forms comprise one or more pharmaceutical compositions comprising, preferably consisting of, the IFN-γ, IL-4, BDNF, IL-2 and/or the immunomodulatory substance(s) and optionally comprising, preferably consisting of, the skin-conditioning agent, preferably as active ingredient(s).

Preferably, such pharmaceutical compositions are any one or more of the pharmaceutical compositions as mentioned in any of the embodiments described herein. More preferably, the pharmaceutical compositions of the topical dosage forms is any one or more of the pharmaceutical compositions as mentioned in any of the embodiments described herein.

Preferably, in the topical dosage forms the immunomodulatory substance(s), IFN-γ, IL-4, BDNF, IL-2 and/or the skin-conditioning agent is in the form of a pharmaceutical composition, more preferably in the form of any of the pharmaceutical compositions as mentioned in any of the embodiments described herein.

Preferably, in the topical dosage forms, the comprised components like the pharmaceutical compositions, substances(s) and/or the agents(s) may be provided each spatially separated within the topical dosage forms, e.g. in separates compartments, layers, chambers, areals, parts, regions, channels provided within the topical dosage forms, or by formulating some or all of the comprised components into particles, or the comprised components may be provided as a mixture without spatial separation, or any combination thereof.

Similarly, in the topical dosage forms, the comprised components, like the pharmaceutical compositions, substances(s), and/or, if applicable, the energizing constituent may be provided each spatially separated within the topical dosage forms, e.g. in separate compartments, layers, chambers, areals, parts, regions, channels, or by formulating some or all of the comprised components into particles, or the comprised components and the energizing constituent may be provided as a mixture without spatial separation. The particles are preferably any particles as mentioned in any of the embodiments described herein.

Hence, the topical dosage forms may comprise without spatial separation a pharmaceutical composition comprising, preferably consisting of, the IFN-γ, IL-4, BDNF, IL-2, the immunomodulatory substance(s) and/or the skin-conditioning agent, preferably as active ingredient(s).

Preferably, in the topical dosage forms, at least one of the comprised components may be provided spatially separated within the injectable topical forms, e.g. in a separate compartment, chamber, areal, part, region, channels provided within the topical dosage forms.

More preferably, the topical dosage forms comprise:
(i) spatially separated
   one or more pharmaceutical compositions comprising, preferably consisting of as active ingredient(s), the IFN-γ, IL-4, BDNF and/or IL-2, and/or
   optionally a further pharmaceutical composition comprising, preferably consisting of as further active ingredient, the immunomodulatory substance(s), and
   optionally a further pharmaceutical composition comprising, preferably consisting of as further active ingredient, the skin-conditioning agent;
or
   (ii) spatially separated
      one or more pharmaceutical compositions comprising, preferably consisting of as active ingredient(s), the IFN-γ, IL-4, BDNF and/or IL-2, and/or
      optionally a further pharmaceutical composition comprising, preferably consisting of as further active ingredient the immunomodulatory substance(s), and
      optionally an energizing constituent;
or
(iii) any combination thereof,
or the injectable dosage forms comprise any of (i) to (iii) in any combination with,
   a pharmaceutical composition without spatial separation, wherein the pharmaceutical composition comprises, preferably consists of as active ingredient(s), the IFN-γ, IL-4, BDNF, IL-2, the immunomodulatory substance(s) and/or the skin-conditioning agent.

Preferably, the topical dosage forms are an article, preferably an article for topical application to the skin.

Preferably, the article has at least one layer holding the pharmaceutical compositions, substances(s) and/or skin-conditioning agent(s). The layer may be made of any pharmaceutically acceptable material or construction known in the art like a matrix, absorbent cotton, reservoir, fabric, felt, fleece or wound gauze. The topical dosage forms may have an adhesive surface for topical application and temporal fixing to the skin.

Preferably, the topical dosage forms and/or the article is a plaster, patch, pad, wadding, padding, dressing, compress, microneedle patch, transdermal patch, time-release plaster and/or bandage. Optionally the topical dosage forms and/or the article particularly plaster, patch, pad, wadding, padding, dressing, compress, microneedle patch, transdermal patch, time-release plaster and/or bandage, is of a multi-compartment type.

Preferably, within the topical dosage forms, the comprised components like the pharmaceutical compositions, substance(s), skin-conditioning agent(s) and/or, if applicable, the energizing constituent are spatially separated within the topical dosage forms. The special separation may be provided by separate compartments, chambers, layers, areals, parts, regions and/or channels provided within the topical dosage forms, or by packaging or formulating one or more of the comprised components into the particles, or by any combination thereof. The particles are preferably any particles as mentioned in any of the embodiments described herein. The compartments, chambers, layers, areals, parts, regions and/or channels may be arranged vertically, for instance by superposition or overlay, allowing for a vertical spatial separation, or they may be arranged horizontally for instance by a distribution in parallel to the surface of the skin when the topical dosage form is applied to the skin of the subject, or any combination thereof.

A multi-compartment type and particularly topical dosage forms of the multi-compartment type may have two or more compartments for spatial separation of the comprised components and/or the comprised energizing constituent. They allow for simultaneous or essentially simultaneous or for a delayed or time-delayed delivery of the comprised components and/or the conditioning energy (as e.g. described below) to the subject with or without previous mutual contacting or mixing of the comprised components. Examples for a topical dosage form of a multi-compartment type are plasters having separate layers superimposed on top of each other or having separate regions or paddings distributed within the plaster in parallel to the surface of the skin when the plaster is applied to the skin of the subject.

By using a multi-compartment type or topical dosage forms providing spatial separation, like topical dosage forms of the multi-compartment type, the comprised components and/or energizing constituent(s) may be stored spatially separately within the topical dosage forms. The delivery of the comprised components and/or the conditioning energy may then be by means of effective surfaces which do not overlap, i.e. are separate to each other. This allows for spatially separate delivery of the pharmaceutical compositions, substance(s), skin-conditioning agent(s) and/or the conditioning energy by using a single topical dosage form. Alternatively, the delivery of the comprised components and/or the conditioning energy may be by means of effective surfaces which at least partially overlap, totally overlap and/or are congruent.

This may for instance be appropriate in case the comprised components, the conditioning energy to be delivered and/or energizing constituent are not miscible or not compatible with each other (e.g. in case the comprised components need to be exposed as less as as possible to the conditioning energy when administered and/or applied. For instance the comprised components may be sensitive to inactivation when exposed to the conditioning energy); a mixture thereof is pharmaceutically not acceptable; the individual comprised components, particularly the substance(s) and/or skin-conditioning agent(s), need different, incompatible formulation conditions to remain stable and active; the substance(s) and/or skin-conditioning agent(s), particularly the substance(s), are only stable in dry form like a lyophilisate and require reconstitution in e.g. a pharmaceutically acceptable diluent directly before delivery to the patient for instance by injection; or the comprised components, particularly the substance(s) and/or skin-conditioning agent(s), are only approved by the authorities in individual formulations or compositions, but not as combination preparations.

Preferably, the topical dosage forms are loaded or pre-loaded with the pharmaceutical compositions, the IFN-γ, IL-4, BDNF, IL-2, immunomodulatory substance(s) and/or the skin-conditioning agent(s), preferably as active ingredient(s). More preferably, the topical dosage forms are exclusively loaded or pre-loaded with one or more of the pharmaceutical compositions, the IFN-γ, IL-4, BDNF, IL-2, the immunomodulatory substance(s) and/or the skin-conditioning agent(s) as active ingredient(s).

More preferably, the topical dosage forms are loaded or pre-loaded with one or more of the pharmaceutical compositions as mentioned in any of the embodiments described herein, preferably as active ingredient(s). More preferably, the topical dosage forms are exclusively loaded or pre-loaded with one or more of the pharmaceutical compositions as active ingredient(s).

Preferably, in any of the embodiments described herein the topical dosage forms are capable of delivering conditioning energy to the skin.

Hence, preferably, in any of the embodiments described herein, the topical dosage forms is an energizing topical dosage form.

Preferably, the topical dosage forms and/or the energizing topical dosage forms are any topical dosage form suitable and/or configured for delivering, administering and/or applying conditioning energy to the skin, preferably to the surface of the skin.

Preferably, the topical dosage forms and/or the energizing topical dosage forms are suitable and/or configured for delivering, administering and/or applying the conditioning energy to any extent and/or any duration as long as it is pharmaceutically acceptable and does not cause lesions and/or a damaging of skin like burning, causing lesions, rupture of capillaries and/or reflexive closure of the capillaries.

Preferably, the topical dosage forms and/or the energizing topical dosage form comprise an energizing constituent for providing the conditioning energy which is to be delivered, administered and/or applied to the skin. Preferably, the energizing constituent is any energizing constituent known to the person skilled in the art, which is pharmaceutically acceptable and/or does not cause lesions and/or a damaging of skin like burning, causing lesions, rupture of capillaries and/or reflexive closure of the capillaries.

Preferably, the energizing constituent is any constituent suitable, preferably configured, for delivering, administering and/or applying the conditioning energy to the skin, preferably to the surface of the skin. Preferably, the energizing constituent is suitable, preferably configured, for delivering, administering and/or applying the conditioning energy to any extent and/or any duration as long as it is pharmaceutically acceptable and does not cause lesions and/or a damaging of skin like burning, causing lesions, rupture of capillaries and/or reflexive closure of the capillaries.

Preferably, the topical dosage forms, the energizing topical dosage forms and/or energizing constituent is suitable, preferably configured, for delivering, administering and/or applying, and/or delivers, administers and/or applies the conditioning energy to the skin by electromagnetic waves, radiation, electric current, heat e.g. by physical processes like conduction due to mechanical contact to the skin, by convection due to entrainment of thermal energy in a flowing fluid, inductive coupling, thermal radiation using electromagnetic waves, visible light, and/or by inducing biochemical processes like by administering and/or applying the skin-conditioning agent as mentioned in any of the embodiments described herein to the skin, or any combination thereof.

The topical dosage forms, the energizing topical dosage forms and/or energizing constituent may be any topical dosage form, energizing topical dosage form and/or energizing constituent, respectively, emitting thermal radiation; emitting infrared radiation; emitting ultrasonic; emitting radiation like visible light; providing electric current to the skin; and/or further comprising, preferably consisting of, a skin-conditioning agent, preferably as further active ingredient(s).

The topical dosage forms, the energizing topical dosage forms and/or energizing constituent may for instance be any means emitting thermal radiation; means emitting infrared radiation; means emitting ultrasonic; means emitting electromagnetic waves; means emitting microwaves; means emitting ultraviolet light; means emitting radiation like visible light; means emitting hot air or warm fluid; means providing electric current to the skin; means providing heat to the skin; a warm or warming object like a heater, a heat pad e.g. equipped with a latent heat storage, an electrical warmer e.g. like tiny version of an electrical hand warmer, a preheated object; means for moxibustion; means for performing work on the skin; means for massage like machines suitable for skin massage like a facial massager and/or vibrating machine; means for generating vibration and/or rubbing; means for providing mechanical agitation to the skin of the subject; means for providing any kind of vacuum or negative pressure to the skin like an apparatus sucking the skin, cupping machine or vacuum pump, means for providing ultrasonic to the skin, a heat accumulator charged before use or based on an exothermic chemical reaction, an activable latent heat storage like a heat pad, a source for electric current, a source for radiation like thermal radiation and/or infrared radiation, a source for ultrasonic, a source for visible light, a source or reservoir for the skin-conditioning agent, etc. or any combination thereof. Preferably, the topical dosage forms, the energizing topical dosage forms and/or energizing is any means emitting thermal radiation; means emitting infrared radiation; means emitting ultrasonic; means emitting electromagnetic waves; means emitting microwaves; means emitting ultraviolet light; means emitting radiation like visible light; means providing electric current to the skin; means providing heat to the skin; a warm or warming object like a heater, a heat pad e.g. equipped with a latent heat storage, an electrical warmer e.g. a tiny version of an electrical hand warmer, a preheated object; means for moxibustion; means for providing ultrasonic to the skin, a heat accumulator charged before use or based on an exothermic chemical reaction, an activable latent heat storage like a heat pad, a source for electric current, a source for radiation like thermal radiation and/or infrared radiation, a source for ultrasonic, a source for visible light, etc. or any combination thereof.

More preferably, the topical dosage forms and/or the energizing topical dosage forms are an article as defined above. Even more preferably, the topical dosage forms, the energizing topical dosage forms, the heating topical dosage forms and/or the article is a heat plaster, heat patch, heat pad, heat wadding, heat padding, heat dressing, heat compress, microneedle heat patch, transdermal heat patch, time-release heat plaster, moxa plaster and/or heat bandage. Optionally the topical dosage forms, the energizing topical dosage forms and/or the article, is of a multi-compartment type as described herein.

The conditioning energy may be any energy known to the person skilled in the art, which is pharmaceutically acceptable and/or does not cause lesions and/or a damaging of skin like burning, causing lesions, rupture of capillaries and/or reflexive closure of the capillaries. For instance, the conditioning energy is in a form of work; heat; electromagnetic radiation; microwaves; thermal radiation; infrared radiation; ultraviolet light; visible light; mechanical agitation; negative pressure; mechanical work; electrical work; electric current; ultrasonic; mechanical agitation; a massage like a manual or machined skin massage using e.g. vibration and/or rubbing of the skin; vibration; rubbing; friction; negative pressure using e.g. sucking like machined, manual or oral sucking; manually or machined generated negative pressure using e.g. cupping particularly dry cupping or cupping without injuring the skin; hot air; hyperthermia and/or warm flowing fluid like warm water, or any combination thereof. Preferably, the conditioning energy is in the form of heat; electromagnetic radiation; thermal radiation; infrared radiation; electrical work; electric current; and/or hyperthermia, or any combination thereof. More preferably, the conditioning energy is in the form heat; electromagnetic radiation; thermal radiation; infrared radiation; and/or hyperthermia, or any combination thereof.

The conditioning energy may be any energy which is pharmaceutically acceptable and does not cause lesions and/or a damaging of skin like burning, causing lesions, rupture of capillaries and/or reflexive closure of the capillaries.

Preferably, the conditioning energy is any energy for modifying a condition of the skin particularly in respect of the amount of PBMCs within the skin, the vasodilation of the capillaries within the skin, the blood volume within the skin, the sO₂ within the skin, the rHb within the skin, the temperature on the skin and/or the redness on the skin.

Preferably, the topical dosage forms, the energizing topical dosage forms and/or energizing constituent is any topical dosage form, energizing topical dosage form and energizing constituent, respectively, suitable for modifying a condition of the skin particularly in respect of the amount of PBMCs within the skin, the vasodilation of the capillaries within the skin, the blood volume within the skin, the sO₂ within the skin, the rHb within the skin, the temperature on the skin and/or the redness on the skin.

In any of the embodiments described herein and particularly for the topical dosage forms, the energizing topical dosage forms and/or energizing constituent the conditioning energy may also be referred to as energy or skin-conditioning energy.

More preferably, the topical dosage forms and/or the energizing topical dosage forms is are heating topical dosage forms.

More preferably, the conditioning energy is heat.

More preferably, the energizing constituent is a heating constituent.

Hence, preferably, in any of the embodiments described herein, the topical dosage forms are heating topical dosage forms.

Preferably, the topical dosage forms and/or the heating topical dosage forms are any topical dosage form suitable and/or configured for delivering, administering and/or applying heat to the skin, preferably to the surface of the skin.

Preferably, the topical dosage forms and/or the heating topical dosage forms are suitable and/or configured for delivering, administering and/or applying the heat to any extent and/or any duration as long as it is pharmaceutically acceptable and does not cause lesions and/or a damaging of skin like burning, causing lesions, rupture of capillaries and/or reflexive closure of the capillaries.

Preferably, the topical dosage forms and/or the heating topical dosage forms comprise a heating constituent for providing the heat which is to be delivered, administered and/or applied to the skin. Preferably, the heating constituent is any heating constituent known to the person skilled in the art, which is pharmaceutically acceptable and/or does not cause lesions and/or a damaging of skin like burning, causing lesions, rupture of capillaries and/or reflexive closure of the capillaries.

Preferably, the heating constituent is any constituent suitable, preferably configured, for delivering, administering and/or applying the heat to the skin, preferably to the surface of the skin. Preferably, the heating constituent is suitable, preferably configured, for delivering, administering and/or applying the heat to any extent and/or any duration as long as it is pharmaceutically acceptable and does not cause lesions and/or a damaging of skin like burning, causing lesions, rupture of capillaries and/or reflexive closure of the capillaries.

Preferably, the topical dosage forms, the heating topical dosage forms and/or heating constituent forms is suitable, preferably configured, for delivering, administering and/or applying, and/or delivers, administers and/or applies the heat to the skin by electromagnetic waves, radiation, electric current, heat e.g. by physical processes like conduction due to mechanical contact to the skin, by convection due to entrainment of thermal energy in a flowing fluid, inductive coupling, thermal radiation using electromagnetic waves, visible light, and/or by inducing biochemical processes like by administering and/or applying the skin-conditioning agent as mentioned in any of the embodiments described herein to the skin, or any combination thereof.

The topical dosage forms, the heating topical dosage forms and/or heating constituent may be any topical dosage form, heating topical dosage forms and/or heating constituent, respectively, emitting thermal radiation; emitting infrared radiation; emitting ultrasonic; emitting radiation like visible light; providing electric current to the skin; and/or further comprising, preferably consisting of, a skin-conditioning agent, preferably as further active ingredient(s).

The topical dosage forms, the heating topical dosage forms and/or heating constituent may for instance be any means emitting thermal radiation; means emitting infrared radiation; means emitting ultrasonic; means emitting electromagnetic waves; means emitting microwaves; means providing electric current to the skin; means emitting ultraviolet light; means emitting radiation like visible light; means providing heat to the skin; a warm or warming object like a heater, a heat pad e.g. equipped with a latent heat storage, an electrical warmer e.g. an electrical hand warmer, a preheated object or a warm tea cup; means for moxibustion; means for providing ultrasonic to the skin, a heat accumulator charged before use or based on an exothermic chemical reaction, an activable latent heat storage like a heat pad, a source for electric current, a source for radiation like thermal radiation and/or infrared radiation, a source for ultrasonic and/or a source for visible light, a source or reservoir for the skin-conditioning agent, etc. Preferably, the topical dosage forms, the heating topical dosage forms and/or heating constituent may for instance be any means emitting thermal radiation; means emitting infrared radiation; means emitting electromagnetic waves; means providing heat to the skin; a warm or warming object like a heater, a heat pad e.g. equipped with a latent heat storage, an electrical warmer e.g. an electrical hand warmer, a preheated object or a warm tea cup; means for moxibustion, a heat accumulator charged before use or based on an exothermic chemical reaction, an activable latent heat storage like a heat pad, a source for radiation like thermal radiation and/or infrared radiation and/or a source for visible light, etc, or any combination thereof.

More preferably, the topical dosage forms and/or the heating topical dosage forms are an article as defined above. Even more preferably, the topical dosage forms, the energizing topical dosage forms, the heating topical dosage forms and/or the article is a heat plaster, heat patch, heat pad, heat wadding, heat padding, heat dressing, heat compress, microneedle heat patch, transdermal heat patch, time-release heat plaster, moxa plaster and/or heat bandage. Optionally the topical dosage forms, the heating topical dosage forms and/or the article, is of a multi-compartment type as described herein.

In any of the embodiments described herein and particularly for the topical dosage forms, the energizing topical dosage forms and/or energizing constituent, the heat may be any form of heat known to the person skilled in the art, which is pharmaceutically acceptable and/or does not cause lesions and/or a damaging of skin like burning, causing lesions, rupture of capillaries and/or reflexive closure of the capillaries. For instance, the heat is in the form of electromagnetic radiation; microwaves; thermal radiation; infrared radiation; electrical work; electric current; ultraviolet light; visible light; hyperthermia and/or warm flowing fluid like warm water, or any combination thereof. Preferably, the conditioning energy is in the form heat; electromagnetic radiation; thermal radiation; infrared radiation; and/or hyperthermia, or any combination thereof.

Preferably, the heat is pharmaceutically acceptable and does not cause lesions and/or a damaging of skin like burning, causing lesions, rupture of capillaries and/or reflexive closure of the capillaries.

Preferably, the heat is an any heat for modifying a condition of the skin particularly in respect of the amount of PBMCs within the skin, the vasodilation of the capillaries within the skin, the blood volume within the skin, the sO₂ within the skin, the rHb within the skin, the temperature on the skin and/or the redness on the skin.

Preferably, the topical dosage forms, the heating topical dosage forms and/or heating constituent is any topical dosage form, heating topical dosage form and heating constituent, respectively, are suitable for modifying a condition of the skin particularly in respect of the amount of PBMCs within the skin, the vasodilation of the capillaries within the skin, the blood volume within the skin, the sO₂ within the skin, the rHb within the skin, the temperature on the skin and/or the redness on the skin.

Preferably,
the conditioning energy and/or the heat is suitable and/or sufficient to generate and/or generates; and/or
the conditioning energy and/or the heat is administered and/or applied to generate; and/or preferably the amount, i.e. to an extent and/or a duration, of the conditioning energy and/or the heat which is administered and/or applied is suitable and/or sufficient to generate; and/or
the topical dosage forms, the energizing topical dosage form, the heating topical dosage form, the energizing constituent and/or the heating constituent is suitable and/or configured to generate and/or generates:
   an accumulation of PBMCs within the skin, wherein it is to be understood that preferably any embodiment or definition thereof which is described under the outline note ‘As further regards step (A-0) for any of the embodiments as described herein’ or in any of the embodiments of the step (A) of the method as described herein is independently and mutatis mutandis applicable; and/or
   a vasodilation of the capillaries within the skin of the subject, wherein it is to be understood that preferably any embodiment or definition thereof which is described under the outline note ‘As further regards step (A-1) for any of the embodiments as described herein’ or in any of the embodiments of the step (A) of the method as described herein is independently and mutatis mutandis applicable; and/or
   an increased blood volume within the skin of the subject, wherein it is to be understood that preferably any embodiment or definition thereof which is described under the outline note ‘As further regards step (A-2) for any of the embodiments as described herein’ or in any of the embodiments of the step (A) of the method as described herein is independently and mutatis mutandis applicable; and/or
   an increased sO₂ and/or an increased rHb within the skin of the subject, wherein preferably the sO₂ is increased by 2% or more and/or the sO₂ is increased by 2%-points or more; and/or the rHb is increased by 2% or more and/or the rHb is increased by 2 AU (arbitrary units) or more, wherein it is to be understood that preferably any embodiment or definition thereof which is described under the outline note ‘As further regards step (A-3) for any of the embodiments as described herein’ or in any of the embodiments of the step (A) of the method as described herein is independently and mutatis mutandis applicable; and/or
   an increased temperature on the skin of the subject, wherein preferably the temperature is increased by 1% or more and/or the temperature is increased by 0.2° C. or more, wherein it is to be understood that preferably any embodiment or definition thereof which is described under the outline note ‘As further regards step (A-4) for any of the embodiments as described herein’ or in any of the embodiments of the step (A) of the method as described herein is independently and mutatis mutandis applicable; and/or
   a redness on the skin of the subject, wherein it is to be understood that preferably any embodiment or definition thereof which is described under the outline note ‘As further regards step (A-5) for any of the embodiments as described herein’ or in any of the embodiments of the step (A) of the method as described herein is independently and mutatis mutandis applicable,
more preferably,
   the vasodilation of the capillaries within the skin,
   the increased blood volume within the skin,
   the increased sO₂ and/or the increased rHb within the skin, and/or
   the increased temperature on the skin,
even more preferably,
   the increased blood volume within the skin,
   the increased sO₂ and/or the increased rHb within the skin, and/or
   the increased temperature on the skin.

If not mentioned otherwise, it is to be understood that any embodiment or definition described herein, particularly any embodiment or definition of the conditioning energy and/or the heat as described herein for step (A-6) of the method in terms of:
the temperature generated on the skin;
the amount of conditioning energy and/or heat delivered, administered and/or applied;
within which time such amount is delivered, administered and/or applied;
the duration the conditioning energy and/or heat is administered and/or applied; and/or
the temperature of the energizing means and/or heating means, is independently and mutatis mutandis applicable to the conditioning energy and/or the heat, respectively, as mentioned for the topical dosage forms, the heating topical dosage form, the energizing constituent and/or the heating constituent.

Furthermore, if not mentioned otherwise, it is to be understood that any embodiment or definition described herein, particularly any embodiment or definition of the energizing means and/or heating means described herein for step (A-6) of the method in terms of:
the temperature generated on the skin;
the amount of conditioning energy and/or heat delivered, administered and/or applied;
within which time such amount is delivered, administered and/or applied;
the duration the conditioning energy and/or heat is administered and/or applied; and/or
the temperature of the energizing means and/or heating means,
is independently and mutatis mutandis applicable to the topical dosage forms, the heating topical dosage forms, the energizing constituent and/or the heating constituent, wherein for that reason, preferably, where applicable, the term ‘energizing means’ is to be understood ‘the topical dosage forms’, ‘the energizing topical dosage forms’ and/or ‘energizing constituent’ and the term ‘heating means’ is to be understood ‘the topical dosage forms’, ‘the heating topical dosage forms’ and/or ‘the heating constituent’.

If not mentioned otherwise, it is to be understood that any embodiment or definition described herein in terms of the topical dosage forms is independently and mutatis mutandis applicable to the energizing topical dosage forms in any of the embodiments of the topical dosage as described herein, and vice versa. Furthermore, if not mentioned otherwise, it is to be understood that any embodiment or definition described herein in terms of the topical dosage forms is independently and mutatis mutandis applicable to the heating topical dosage forms in any of the embodiments of the topical dosage form as described herein, and vice versa.

Preferably, the topical dosage forms are suitable and/or configured for intraepidermal, intradermal and/or subcutaneous, more preferably for intradermal delivery of the substance(s) and/or skin-conditioning agent(s), preferably the immunomodulatory substance(s), IFN-γ, IL-4, BDNF and/or IL-2.

Preferably, the topical dosage forms are suitable and/or configured to deliver the substance(s), particularly the immunomodulatory substance(s), like the IFN-γ, IL-4, BDNF, IL-2, into a sub-topical layer of the skin, more preferably, into the epidermis, dermis and/or subcutis, even more preferably the dermis, of the skin and/or 6 mm or less to 0.1 mm or more, more preferably 4 mm or less to 0.3 mm or more, even more preferably 3 mm or less to 0.5 mm or more in skin thickness direction into the skin, when measured from the skin surface, preferably when measured perpendicular to the skin surface towards the bones.

Preferably, the topical dosage forms have an effective surface.

The expression “effective surface” as mentioned in any of the embodiments described herein relates to a surface of the topical dosage forms and/or medical devices which delivers or releases and/or which is suitable and/or configured to deliver or release the substance(s), skin-conditioning agent(s), conditioning energy and/or heat to the skin of the subject, when the topical dosage forms and/or medical devices are applied to the skin.

The expression “effective surface area” as mentioned in any of the embodiments described herein relates to the area of the effective surface. Usually, the size and/or shape of the application area of the skin usually corresponds to the size and/or the shape of the effective surface area. Preferably, it is to be understood that any description and definition of the size and/or the shape of the application area as mentioned in any of the embodiments of the method as described herein is independently and mutatis mutandis applicable to the size and/or the shape of the effective surface area as mentioned in any of the embodiments of the topical dosage forms and/or medical devices described herein.

Preferably, the amounts or total amounts of the substance(s) of the substance(s), the condition agents(s) and/or the conditioning energy is delivered to the skin of the subject by means of the effective surface of the topical dosage forms.

Preferably, the amounts or total amounts which the topical dosage forms are suitable and/or configured to deliver, are delivered to the skin of the subject by means of the effective surface of the topical dosage forms.

The effective surface may have an area (effective surface area) of any particular size. Preferably the size and/or shape of the effective surface area is as defined herein.

By means of the effective surface having an effective surface area, the substance(s), agents, conditioning energy and/or heat are via the skin of the application area administered and/or delivered to the skin of the skin area and finally the accumulation of PBMCs, vasodilation, increased blood volume, increased sO₂, increased rHb, increased temperature and/or redness as mentioned in any of the embodiments described herein may be generated on and/or within the skin of the skin area.

More preferably, in any of the embodiments of the pharmaceutical compositions, the injectable dosage forms, the topical dosage forms and/or the medical devices described herein, the effective surface area is in case of the skin-conditioning agent, the conditioning energy and/or the heat an effective surface area [a].

More preferably, in any of the embodiments of the pharmaceutical compositions, the injectable dosage forms, the topical dosage forms and/or the medical devices described herein, the effective surface area is in case of the immunomodulatory substance(s), particularly IFN-γ, IL-4 and BDNF an effective surface area [b] and/or [b₁].

More preferably, in any of the embodiments of the pharmaceutical compositions, the injectable dosage forms, the topical dosage forms and/or the medical devices described herein, the effective surface area is in case of the immunomodulatory substance(s), particularly is for IL-2 an effective surface area [c].

Preferably, in any the embodiments of the pharmaceutical compositions, the injectable dosage forms, the topical dosage forms and/or the medical devices described herein, the effective surface is of a surface area. In other words, the effective surface has an area designated as effective surface area. Preferably, it is to be understood that any description and definition of the size and/or the shape of the application area as mentioned in any of the embodiments of the method as described herein is independently and mutatis mutandis applicable to the the size and/or the shape of the effective surface area as mentioned in any the embodiments of the pharmaceutical compositions, the injectable dosage forms, the topical dosage forms and/or the medical devices described herein.

Preferably, the effective surface area, specifically the effective surface area [a], [b], [b₁] and/or [c], is independently in the range of preferably 1.5 m² or less and 0.01 cm² or more, more preferably 1000 cm² or less and 0.01 cm² or more, even more preferably 500 cm² or less and 0.01 cm² or more, still more preferably 100 cm² or less and 0.02 cm² or more, still even more preferably 50 cm² or less and 0.2 cm² or more, further preferably 25 cm² or less and 0.5 cm² or more, even further preferably 10 cm² or less and 1 cm² or more, still further preferably 5 cm² or less and 2 cm² or more. The the effective surface area as mentioned in any of the embodiments is preferably measured as described in the Materials and Methods' section ‘Measurement of areas and distances relating to the skin’.

If not mentioned otherwise, it is to be understood that any embodiment or definition described herein in terms of the application area, particularly the application area [a], [b], [b₁] and/or [c], is independently and mutatis mutandis applicable to the effective surface area, particularly the effective surface area [a], [b], [b₁] and/or [c], as mentioned in any of the embodiments of the described herein, particularly in any of the embodiment of the pharmaceutical compositions, the injectable dosage forms, the topical dosage forms and/or the medical devices. Particularly, it is to be understood that any embodiment or definition described herein in terms of the application area, particularly the application area [a], [b], [b₁] and/or [c], regarding the size, shape, overlapping and/or the area sum is independently and mutatis mutandis applicable to the effective surface area, particularly the effective surface area [a], [b], [b₁] and/or [c], as mentioned in any of the embodiments described herein.

The topical dosage forms may be for single or repeated use, preferably for single use.

Even more preferably, the topical dosage forms, energizing topical dosage form and/or heating topical dosage are suitable and/or configured for use in a method for treating and/or preventing an inflammatory disease, immunological disease and/or autoimmunological disease in a subject,
wherein the method comprises, preferably consists of, a step (A), wherein step (A) is selected from one or more of steps:
(A-0) generating an accumulation of PBMCs within the skin of the subject;
(A-1) generating a vasodilation of the capillaries within the skin of the subject;
(A-2) generating an increased blood volume within the skin of the subject;
(A-3) generating an increased sO₂ within the skin and/or an increased rHb within the skin of the subject;
(A-4) generating an increased temperature on the skin of the subject;
(A-5) generating a redness on the skin of the subject;
(A-6) administering conditioning energy to the skin of the subject;
(A-7) administering a skin-conditioning agent to the skin of the subject; and/or
(A-8) administering PBMCs into the skin of the subject,
and the method further comprises the step(s) of;
(B) administering the immunomodulatory substance(s) to the skin of said subject; and/or
(C) administering the immunomodulatory substance(s) to the skin of the subject,
wherein the immunomodulatory substance(s) administered in step (C) is different from the immunomodulatory substance(s) administered in step (B),
wherein step (A) is executed by the energizing constituent of the topical dosage forms and/or energizing topical dosage forms or by the heating constituent of the topical dosage forms and/or heating topical dosage form and
wherein at least one of the steps (B) or (C) is executed by means of immunomodulatory substance(s), particularly the IFN-γ, IL-4, BDNF and/or IL-2 comprised in and/or delivered and/or administered by the topical dosage forms, energizing topical dosage form and/or heating topical dosage.

If not mentioned otherwise it is to be understood that the expression “the topical dosage forms” refers to any or all of the topical dosage forms, particularly the topical dosage form (I), (II) and/or (X), more particularly the topical dosage form (I) and/or (II), as mentioned in any of the embodiments described herein. Furthermore, if not mentioned otherwise, it is to be understood that any of the embodiments described herein in plural with reference to the topical dosage forms refers independently to any of the topical dosage forms, particularly the topical dosage form (I), (II) and/or (X), more particularly the topical dosage form (I) and/or (II), as mentioned in any of the embodiments described herein.

It is further to be understood that any of the embodiments mentioned under the outline note ‘As further regards independently any or all of the topical dosage forms, particularly the topical dosage form (I), (II) and/or (X), for any of the embodiments as described herein’ is independently applicable and/or combinable with any of the embodiments of the topical dosage forms, particularly as described under the outline note ‘As further regards independently any or all of the pharmaceutical compositions, the injectable dosage forms, the topical dosage forms, the medical devices and/or the kits of parts for any of the embodiments as described herein’.

As further regards the medical device (100) for any of the embodiments as described herein

The method for treating and/or preventing an inflammatory disease, an immunological disease and/or an autoimmunological disease of a subject, as described above, which can include steps of administering an immunomodulatory substance(s) and/or a skin-condition agent, can require frequent medical treatments and/or an adaption of the treatment in such a way that medical staff passes on the treatment to patients suffering from the disease.

To make sure that the treatment is performed in the right way by the patient and/or in the correct order of steps and/or by using the prescribed quantity of the pharmaceutical compositions or substances, a medical device was invented and is described herein.

As described above, the method for treating and/or preventing an inflammatory disease, immunological disease and/or autoimmunological disease in a subject,
wherein the method comprises, preferably consists of, a step (A), wherein step (A) is selected from one or more of steps:
(A-0) generating an accumulation of PBMCs within the skin of the subject;
(A-1) generating a vasodilation of the capillaries within the skin of the subject;
(A-2) generating an increased blood volume within the skin of the subject;
(A-3) generating an increased sO₂ within the skin and/or an increased rHb within the skin of the subject;
(A-4) generating an increased temperature on the skin of the subject;
(A-5) generating a redness on the skin of the subject;
(A-6) administering conditioning energy to the skin of the subject;
(A-7) administering a skin-conditioning agent to the skin of the subject; and/or
(A-8) administering PBMCs into the skin of the subject,
and the method further comprises the step(s) of;
(B) administering the immunomodulatory substance(s) to the skin of said subject; and/or
(C) administering the immunomodulatory substance(s) to the skin of the subject,
wherein the immunomodulatory substance(s) administered in step (C) is different from the immunomodulatory substance(s) administered in step (B).

Using other words, the immunomodulatory substance of step (B) can be a first immunomodulatory substance; and the immunomodulatory substance of step (C) can be a second immunomodulatory substance.

As described above, the method for treating and/or preventing is very effective in the treatment and/or prevention of an inflammatory disease, immunological disease and/or autoimmunological disease and has, amongst others as stated above, no adverse side effects. The beneficial effects of the method for treating and/or preventing are outlined in detail above.

The present invention is easy and quick to perform, for example, because steps (A) and (B) and/or (C) are performed on and/or within the skin. The skin has the advantage to be easily accessible for treatment and administration. Furthermore advantageous is that treatments on the skin, like administering balms, crèmes or plasters to the skin, and/or administering pharmaceuticals that are inserted into the skin, like injections, are generally of lower health risk for a subject like for instance intravenous injections. The method for treating and/or preventing provides an off the shelf therapy without a requirement of specialized health care centres. This allows for self-application by the affected subject or the owner of an animal and has a high patient compliance.

Thus, affected subjects may respectively self-apply the method steps of any one of the methods for treating and/or preventing and/or embodiments of the method as described above, which can result in a lower cost of the treatment.

The enablement of the method for treating and/or preventing an inflammatory disease, immunological disease and/or autoimmunological disease in a subject is demonstrated in the examples related to this method.

Aspects of the present invention are related to a medical device, a medical device suitable for use in a method, a use of the medical device and a kit of parts with subject matter as described in the independent claims.

Advantageous modifications of the invention are stated in the dependent claims. All combinations of at least two of the features disclosed in the description, the claims, and the FIGS. fall within the scope of the invention. In order to avoid repetition, features disclosed in accordance with the method shall also apply and be claimable in accordance with mentioned systems.

In this entire description of the invention, the sequence of procedural steps is presented in such a way that the process is easily comprehensible. However, the skilled person will recognize that many of the process steps can also be executed in a different order and lead to the same or a corresponding result. In this sense, the sequence of the process steps can be changed accordingly. Some features are provided with counting words to improve readability or to make the assignment more clear, but this does not imply the presence of certain features.

According to an aspect of the invention a medical device (100) is provided including
a skin-conditioning unit; and
an applicator unit;
wherein the skin-conditioning unit and the applicator unit can be configured to cooperatively interact to administer to a skin of a subject an amount of a first therapeutic agent by means of the applicator unit with an amount of conditioning measures by means of the skin-conditioning unit.

Alternatively or additionally, according to an aspect a medical device (100) is provided including
a skin-conditioning unit; and
an applicator unit,
wherein the skin-conditioning unit and the applicator unit can be configured to act to a skin of a subject.

In the following the medical device (100)

Acting to the skin of the subject can be made by means of the skin-conditioning unit and/or the applicator unit and/or particularly by means of the applicator unit.

Acting to the skin of the subject can include any physical and/or chemical and/or pharmaceutical measures and/or medical measures, particularly interacting with the skin of the subject, to change a condition of the skin and/or for provision of a medical treatment.

Acting to the skin of the subject can mean that the skin-conditioning unit and/or the, preferably first, applicator unit are configured to act to an area of the skin of the subject in that respect that, if the skin-conditioning unit is acting to a first area of the skin of the subject and the, preferably first, applicator unit is acting to a second area of the skin of the subject, then the first area of the skin and the second area of the skin are at least partially overlap each other and/or that the first area of the skin and the second area of the skin are adjacent to another for associated application of the conditioning measures and the first therapeutic agent.

The associated administering can mean that the medical device is configured in such a way that the respective administering of conditioning measures with the administration of the first therapeutic agent can support any one of the methods for treating and/or preventing an inflammatory disease, immunological disease and/or autoimmunological disease of a subject successfully.

Using other words the conditioning measures administered to the skin can support a medical effectiveness of administering the first therapeutic agent and/or a respective therapeutic agent. An effect of this associated administering can be that the first and/or another respective therapeutic agent can be administered having an extremely low concentration and/or can be administered by a small quantity of the active ingredient, particularly to avoid a systemic increase if the active ingredient of the therapeutic agent is an immunomodulatory substance(s).

The term skin is used to describe an affected area of the skin.

Acting to the skin of the subject, particularly by means of the applicator unit, can comprise administering a pharmaceutical or a therapeutic agent to the surface of the skin and/or to subcutaneous administering a pharmaceutical or therapeutic agent, particularly to a layer of the skin.

Particularly, acting to a skin of the subject, particularly by means of the applicator unit, can include acting to a surface of the skin and/or penetrating the skin.

Penetrating the skin can include any penetration of the skin including penetration using a single or a plurality of needles and/or using a single or a plurality of hollow needles and/or using a single or a plurality syringes and/or penetration of the skin by pressure injection of a fluid pumped.

Acting to the surface of the skin by means of the skin-conditioning unit can include any conditioning energy impacting the skin, particularly impacting the surface of the skin of the subject, as e.g. electromagnetic radiation and/or infrared radiation and/or heat and/or heat in contact with the surface of the skin. Additionally or alternatively, acting to the surface of the skin by means of the skin-conditioning unit can include any mechanical effects impacting the skin, particularly mechanical effects on the surface of the skin.

The skin-conditioning unit and, the, particularly first, applicator unit can cooperatively interact to act to the skin.

Cooperative interacting of the two units can be an interaction of the skin-conditioning unit and the, particularly first, applicator unit in such a way, that the skin-conditioning unit and the, particularly first, applicator unit are configured, particularly by means of a coupling unit, for mechanically, signally, wirelessly and/or electrically coupling of the two units, to interact in such a way, that both of the units are administering cooperatively, particularly synergistically, the amount of the first therapeutic agent by means of the applicator unit with an amount of conditioning measures by means of the skin-conditioning unit, to the skin of the subject, particularly if the medical device is operated by a user of the medical device.

Preferably, in any one of the embodiments and methods for treating and/or preventing as described herein is to be understood, that the individual effects or sub-effects generated by steps (A), (B) and/or (C) should at least partially and/or totally overlap in time as well as spatial. For instance the effect of the steps can be e.g. to be mutual dependent, supportive, interactive, complementary, additive, synergetic and/or act by any other way to gather a medical effect. Hence, is to be understood that the method should be performed accordingly.

Using other words, cooperative interacting can mean the acting of the skin-conditioning unit and the acting of the, particularly first, application unit is coordinated and/or synchronized to impact the skin of the subject according to a series and/or a sequence of steps of a therapeutic method like for instance the methods for treating and/or preventing as described herein.

Therefore, the skin-conditioning unit and the, particularly first, applicator unit can be configured to cooperatively interact to act to the skin. Particularly, the skin-conditioning unit and the applicator unit can be configured to cooperatively interact to administer an amount of a first therapeutic agent by means of the, particularly first, applicator unit with an amount of conditioning measures by means of the skin-conditioning unit.

Preferably, for the cooperative interacting of the skin-conditioning unit and the applicator unit, the skin-conditioning unit and the applicator unit can be seen as to be coupled in respect to administering the related therapeutic agent with the conditioning measures within a defined and specified time interval and a mutual distance of the administering to the skin to make sure that the respective therapeutic agent and the conditioning measures are administered in respect to the area of the skin.

The amount of conditioning measures can be determined by a modification of the condition of the skin of the second area of the skin necessary for therapeutic successful administering of the first therapeutic agent and/or a second therapeutic agent and/or a third therapeutic agent to the first area of the skin and/or a second area of the skin and/or a third area of the skin respectively.

Preferably, the amount of the first therapeutic agent, and/or the second therapeutic agent, and/or third therapeutic agent respectively, can be determined by a therapeutic measure of a specific disease, which is treated using the device.

The disease may for instance the inflammatory disease, immunological disease and/or auto immunological disease as described herein.

Preferably, the amount of the conditioning measures can include a duration of the conditioning measures and/or a quantity of the conditioning measures, depending of the kind of the measure.

A skin-conditioning unit can be a means for supplying conditioning energy to surface of a skin of a subject. Preferably, the conditioning energy can be supplied to the surface of the skin for modification of a condition of the skin and/or for preparation of a condition of the skin. The condition of the skin can be modified and/or prepared to enable and/or to increase an effectiveness of another acting to the skin of the subject, particularly by means of the, particularly first, applicator unit.

Alternatively or additionally, acting to the skin of the subject for modification and/or preparation of the condition of the skin can promote further steps of the method.

The applicator unit can be configured to apply a first therapeutic agent and/or a first pharmaceutical to the skin and/or to an area of the skin. Wherein preferably the area of the skin the skin-conditioning unit is acting on is the same and/or overlapping and/or cooperatively close and/or adjacent to a further area of the skin the applicator unit is acting on. The medical device can include a plurality of applicator units.

The first or any respective applicator unit can be configured, by a first or any respective reservoir of the first, or any respective applicator unit to accommodate the first, or any respective therapeutic agent.

The medical device can be a means, which is configured to run and/or to support and/or for use in at least parts of the steps of any one of the methods for treating and/or preventing and/or any one of the embodiments of the method for treating and/or preventing as presented above, particularly to run and/or support and/or for use in at least parts of the steps of any of the method for treating and/or preventing an inflammatory disease, immunological disease and/or autoimmunological disease in a subject, as described above.

As described above, the method for treating and/or preventing can comprises the steps of:
(A) which is selected from one or more of steps (A-0), (A-1), (A-2), (A-3), (A-4), (A-5), (A-6), (A-7) and/or (A-8); and
the method can further comprises the step(s) of:
(B) administering a, preferably first, immunomodulatory substance(s) to the skin of the subject; and/or
(C) administering a, preferably second, immunomodulatory substance(s) to the skin of the subject,
wherein the immunomodulatory substance(s) administered in step (C) is different from the immunomodulatory substance(s) administered in step (B).

The medical device can be configured to accommodate, particularly within the applicator unit, the immunomodulatory substance(s), IFN-γ and/or IL-4 and/or BDNF and/or IL-2, which are particularly for use and/or suitable, preferably configured, for use in any one, two or all of steps (B), (B₁) and/or (C) of any one of the methods as described above.

The medical device can be configured to accommodate, particularly within the skin-conditioning unit, the skin-conditioning agent and/or the energizing means, or the topical dosage forms, which are particularly for use and/or suitable, preferably configured, for use in step (A) of the method.

Advantageously, the medical device can be configured for use and/or can be suitable, for use in the method as mentioned in any one of the methods and/or embodiments of the present invention as described above

The embodiments of the medical device as described here can be suitable, preferably configured, to accomplish, perform and/or effect one or more of steps (A), (B), (B₁) and/or (C), of any one of the, preferably medical, methods as described above, depending on which steps are applicable. Particularly the medical device can be configured two be suitable, to accomplish, perform and/or effect one or more or more of steps (A), (B), (B₁) and/or (C), even more preferably steps (A) and (B); steps (A) and (C); steps (B) and (C); steps (A) and (B₁); and/or steps (B₁) and (C).

It is more preferred, that the medical device can be configured for use and/or suitable, for use in the method are suitable, preferably configured, to accomplish, perform and/or effect all steps the method including, step (A) and one, two or all of steps (B), (B₁) and/or (C), depending on which steps are applicable.

The medical device provides, amongst other, the advantage that no extensive training of a user of the medical device is demanded. In addition, inadequate training of the user is avoided in performing the method by using the medical device, and correct execution of the method is ensured. The medical device can provide an easy handling, which can be particularly important for users having impaired manual skills as it is for instance with rheumatological diseases frequently the case. Additionally or alternatively, a fear of self-application or self-injection errors can be taken away and a lack of treatment adherence can be improved or even overcome due to a simple and uncomplicated application without a visit to the doctor.

The amount of the first therapeutic agent is an amount of the first therapeutic agent, which is foreseen to act to the skin of a subject for successfully accomplishing the therapeutic method. Particularly, the amount of the first therapeutic agent can be specified in such a way as to support at least one step of a medical treatment to the subject. The acting to the skin of the subject can include an administering of the first therapeutic agent to the skin of the subject.

As already described within this specification, preferably, in any one of the methods for treating and/or preventing and/or embodiments as described herein, particularly in step (B), (B₁) and/or (C), the first therapeutic agent, as e.g. the immunomodulatory substance(s) is administered in an amount that causes a local activation, preferably an effective local activation of the respective receptor of the immunomodulatory substance(s), and/or causes a local generation, preferably an effective local generation of the immunomodulatory substance(s), preferably in the subject. Preferably, the activation and/or generation, more preferably the effective generation and/or effective activation is only local but not systemic in the subject.

Hence, the present invention is based on the further surprising finding that the immunomodulatory substance(s) can be administered in extremely low concentrations while still being effective.

Hence, preferably, in any one of the methods for treating and/or preventing and/or related embodiments described herein it is intended to administer the immunomodulatory substance(s) in an amount low enough to avoid a systemic increase of the concentration of the immunomodulatory substance(s) in the subject's body which systemic increase is effective. Thereby, the generation of an increased concentration of the immunomodulatory substance(s) for instance at sites of inflammation like inflamed joint shall be avoided.

The amount of conditioning measures is an amount of the conditioning measures, which is foreseen to act to the skin of a subject for successfully accomplishing of the therapeutic method. Particularly, the amount of the conditioning measures can be specified in such a way as to support at least another step of the medical treatment to the subject.

As already described above, for instance step (A-6) the methods for treating and/or preventing and/or related embodiments thereof described in this specification requires administering conditioning energy to the skin of the subject, which can be defined as a conditioning measures, wherein the conditioning energy is administered and/or applied to the surface of the skin of the subject.

The conditioning measures and/or the conditioning energy may be any measure or energy known to the person skilled in the art, which is pharmaceutically acceptable and/or does not cause lesions and/or a damaging of skin like burning, causing lesions, rupture of capillaries and/or reflexive closure of the capillaries.

For instance, the conditioning measures and/or conditioning energy is in a form of work; heat; electromagnetic radiation; microwaves; thermal radiation; infrared radiation; ultraviolet light; visible light; mechanical agitation; mechanical work; electrical work; electric current; ultrasonic; mechanical agitation; a massage like a manual or machined skin massage using e.g. vibration and/or rubbing of the skin; vibration; rubbing; friction; negative pressure e.g. using sucking like machined, manual or oral sucking; manually or machined generated negative pressure using e.g. cupping particularly dry cupping or cupping without injuring the skin; hot air; hyperthermia and/or warm flowing fluid like warm water, or any combination thereof.

Preferably, the conditioning measures and/or conditioning energy is any measure or energy for modifying a condition of the skin particularly in respect of the amount of PBMCs, the vasodilation of the capillaries within the skin, the blood volume within the skin, the sO₂ within the skin, the rHb within the skin, the temperature on the skin and/or the redness on the skin.

Preferably, the conditioning measures and/or the conditioning energy may be administered and/or applied to the skin to any extent and/or any duration as long as it is pharmaceutically acceptable and does not cause lesions and/or a damaging of skin like burning, causing lesions, rupture of capillaries and/or reflexive closure of the capillaries.

The conditioning energy may also be referred to as energy or skin-conditioning energy or conditioning measures.

As already described, preferably, the skin-conditioning unit, which is also described as energizing means, is suitable, preferably configured, for delivering, administering and/or applying, and/or delivers, administers and/or applies the conditioning energy to the skin by work, electromagnetic waves, radiation, electric current, heat e.g. by physical processes like conduction due to mechanical contact to the skin, by convection due to entrainment of thermal energy in a flowing fluid, mechanical coupling, inductive coupling, thermal radiation using electromagnetic waves, visible light, mechanical agitation, negative pressure and/or by inducing biochemical processes like by administering and/or applying the skin-conditioning agent as mentioned in any one of the embodiments of the present invention described herein to the skin, or any combination thereof.

The skin-conditioning unit and/or energizing means may be an apparative energizing means or a non-apparative energizing means, preferably an apparative energizing means.

Hence, the skin-conditioning unit, energizing means and/or apparative energizing means may for instance be any means emitting thermal radiation; means emitting infrared radiation; means emitting ultrasonic; means emitting electromagnetic waves; means emitting microwaves; means emitting ultraviolet light; means emitting radiation like visible light; means emitting hot air or warm fluid; means providing electric current to the skin; means providing heat to the skin; a warm or warming object like a heater, a heat pad e.g. equipped with a latent heat storage, an electrical warmer e.g. an electrical hand warmer, a preheated object or a warm tea cup; means for moxibustion; means for performing work on the skin; means for massage like machines suitable for skin massage like a facial massager and/or vibrating machine; means for generating vibration and/or rubbing; means for providing mechanical agitation to the skin of the subject; means for providing any kind of vacuum or negative pressure to the skin like an apparatus sucking the skin, cupping glasses, cupping machine or vacuum pump; means for providing ultrasonic to the skin; heat pad, heat patch, heat wadding, heat padding, heat dressing, heat compress, moxa plaster and/or heat bandage; an energizing topical dosage form as mentioned in any one of the embodiments of the present invention described herein, preferably a heating topical dosage form as mentioned in any one of the embodiments of the present invention described; and/or a medical device according to any of the embodiments according to the present invention as described herein, etc. Furthermore, the energizing means and/or the non-apparative energizing means may be for instance warm flowing fluid like water e.g. when run over the skin; manual applications like moving hands of a masseur performing e.g. a manual skin massage using vibration, rubbing or generating a sucking action on the skin.

More preferably, the conditioning measures, respectively the conditioning energy, delivered, administered and/or applied is heat.

Preferably, the heat is pharmaceutically acceptable and/or does not cause lesions and/or a damaging of skin like burning, causing lesions, rupture of capillaries and/or reflexive closure of the capillaries.

For instance, the heat administered and/or applied may be in the form of electromagnetic radiation; thermal radiation; microwaves; infrared radiation; hot air; hyperthermia; warm flowing fluid like warm water.

Preferably, the skin-conditioning unit and/or the energizing means is a heating means.

The heat is administered and/or applied by any heating means known to the person skilled in the art, which heating means is pharmaceutically acceptable and/or does not cause lesions and/or a damaging of skin like burning, causing lesions, rupture of capillaries and/or reflexive closure of the capillaries.

Hence, the skin-conditioning unit may be configured as an energizing means, more preferably as a heating means.

Preferably, the skin-conditioning unit, which is configured as a heating means is suitable, preferably configured, for delivering, administering and/or applying and/or delivers, administers and/or applies the heat to the skin by physical processes like conduction due to mechanical contact to the skin, by convection due to entrainment of thermal heat in a flowing fluid and/or by thermal radiation using electromagnetic waves.

Preferably, the skin-conditioning unit, the energizing means and/or the heating means is suitable, preferably configured, for delivering, administering and/or applying and/or delivers, administers and/or applies the heat to the skin by physical processes like conduction due to mechanical contact to the skin, by convection due to entrainment of thermal heat in a flowing fluid and/or by thermal radiation using electromagnetic waves.

Hence, the skin-conditioning unit, the energizing means and/or the heating means can for instance be any means emitting thermal radiation; means emitting infrared radiation; means emitting ultrasonic; means emitting electromagnetic waves; means emitting microwaves; means emitting ultraviolet light; means emitting radiation like visible light; means emitting hot air or warm fluid; means providing electric current to the skin; means providing heat to the skin; a warm or warming object like a heater, a heat pad e.g. equipped with a latent heat storage, an electrical warmer e.g. an electrical hand warmer, a preheated object or a warm tea cup; means for moxibustion; and/or means for providing ultrasonic to the skin.

Preferably, the amount of conditioning measures, the amount of conditioning energy and/or the amount of heat, i.e. to an extent and/or a duration, is suitable and/or sufficient to generate an accumulation of PBMCs within the skin and/or generate an increased blood volume within the skin and/or generate an increased sO₂ and/or an increased rHb within the skin, and/or generate an increased temperature on the skin and/or generate a redness on the skin.

Particularly,
the conditioning energy and/or the heat is suitable to generate and/or generates; and/or
the medical devices, the skin-conditioning unit, the energizing means and/or heating means is suitable and/or configured to generate and/or generates; and/or
the administering of conditioning measures to the skin of the subject can, as an example, result in the following effects:
   an accumulation of PBMCs within the skin, wherein it is to be understood that preferably any embodiment or definition thereof which is described under the outline note ‘As further regards step (A-0) for any of the embodiments as described herein’ or in any of the embodiments of the step (A) of the method as described herein is independently and mutatis mutandis applicable; and/or
   a vasodilation of the capillaries within the skin of the subject, wherein it is to be understood that preferably any embodiment or definition thereof which is described under the outline note ‘As further regards step (A-1) for any of the embodiments as described herein’ or in any of the embodiments of the step (A) of the method as described herein is independently and mutatis mutandis applicable; and/or
   an increased blood volume within the skin of the subject, wherein it is to be understood that preferably any embodiment or definition thereof which is described under the outline note ‘As further regards step (A-2) for any of the embodiments as described herein’ or in any of the embodiments of the step (A) of the method as described herein is independently and mutatis mutandis applicable; and/or
   an increased sO₂ and/or an increased rHb within the skin of the subject, wherein preferably the sO₂ is increased by 2% or more and/or the sO₂ is increased by 2%-points or more; and/or the rHb is increased by 2% or more and/or the rHb is increased by 2 AU (arbitrary units) or more, wherein it is to be understood that preferably any embodiment or definition thereof which is described under the outline note ‘As further regards step (A-3) for any of the embodiments as described herein’ or in any of the embodiments of the step (A) of the method as described herein is independently and mutatis mutandis applicable; and/or
   an increased temperature on the skin of the subject, wherein preferably the temperature is increased by 1% or more and/or the temperature is increased by 0.2° C. or more, wherein it is to be understood that preferably any embodiment or definition thereof which is described under the outline note ‘As further regards step (A-4) for any of the embodiments as described herein’ or in any of the embodiments of the step (A) of the method as described herein is independently and mutatis mutandis applicable; and/or
   a redness on the skin of the subject, wherein it is to be understood that preferably any embodiment or definition thereof which is described under the outline note ‘As further regards step (A-5) for any of the embodiments as described herein’ or in any of the embodiments of the step (A) of the method as described herein is independently and mutatis mutandis applicable,
more preferably,
   the vasodilation of the capillaries within the skin,
   the increased blood volume within the skin,
   the increased sO₂ and/or the increased rHb within the skin, and/or
   the increased temperature on the skin,
even more preferably,
   the increased blood volume within the skin,
   the increased sO₂ and/or the increased rHb within the skin, and/or
   the increased temperature on the skin.

Preferably, the medical device, the skin-conditioning unit, the energizing means and/or heating means is suitable, preferably configured, to generate and/or generates, a temperature on the skin of 25° C. or more, more preferably 27° C. or more, more preferably 28° C. or more, even more preferably 29° C. or more, still more preferably 30° C. or more, still even more preferably 31° C. or more, further preferably 32° C. or more, preferably when measured as described in the Materials and Methods' section ‘Thermal measurement and skin reddening’. There is no upper limit for temperature on the skin generated as long as no burning lesions and/or damaging of skin like burning, causing lesions, rupture of capillaries and/or reflexive closure of the capillaries is caused. However, usually, the temperature on the skin generated is 65° C. or less, preferably 58° C. or less, more preferably 50° C. or less, still more preferably 45° C. or less, still even more preferably 42° C. or less, further preferably 41° C. or less. More preferably, the temperature on the skin generated is in the range of 25° C. or more and 65° C. or less, more preferably 27° C. or more and 58° C. or less, even more preferably 28° C. or more and 50° C. or less, still more preferably 29° C. or more and 45° C. or less, still even more preferably 30° C. or more and 45° C. or less, further preferably 31° C. or more and 42° C. or less, even further preferably 32° C. or more and 41° C. or less.

Preferably, the amount of conditioning measures, the amount of conditioning energy and/or amount of heat administered and/or applied is 0.1 kJ/cm² skin or more (‘kJ/cm² skin’ is in its meaning equivalent to ‘kilojoule/one square centimetre of the skin), more preferably 0.3 kJ/cm² skin or more, even more preferably 0.6 kJ/cm² skin or more, still more preferably 1 kJ/cm² skin or more, still even more preferably 2 kJ/cm² skin or more, further preferably 3 kJ/cm² skin or more, even further preferably 4 kJ/cm² skin or more, still further preferably by 6 kJ/cm² skin or more. There is no upper limit for the amount of conditioning energy as long as no burning lesions and/or damaging of skin is caused.

However, usually, the amount of conditioning measures, the amount of conditioning energy and/or amount of heat administered and/or applied is 60 kJ/cm² skin or less, preferably 50 kJ/cm² skin or less, even more preferably 40 kJ/cm² skin or less, still more preferably kJ/cm² skin or less, still even more preferably 15 kJ/cm² skin or less, further preferably 10 kJ/cm² skin or less. Preferably, the amount of conditioning energy administered and/or applied is in the range of 0.1 kJ/cm² skin or more and 60 kJ/cm² skin or less, more preferably 0.3 kJ/cm² skin or more and 60 kJ/cm² skin or less, even more preferably by 0.6 kJ/cm² skin or more and 50 kJ/cm² skin or less, still more preferably by 1 kJ/cm² skin or more and 50 kJ/cm² skin or less, still even more preferably by 2 kJ/cm² skin or more and kJ/cm² skin or less, further preferably by 3 kJ/cm² skin or more and 30 kJ/cm² skin or less, even further preferably by 4 kJ/cm² skin or more and 15 kJ/cm² skin or less, still further preferably by 6 kJ/cm² skin or more and 10 kJ/cm² skin or less.

Preferably, the conditioning measures, respectively of the amount of conditioning energy and/or amount of heat, within 60 min or less, more preferably 30 min or less, even more preferably 15 min or less, still more preferably 5 min or less, still even more preferably 2 min or less. Usually, it is within 1 sec or more, more preferably 5 sec or more, even more preferably 10 sec or more, still more preferably sec or more, still even more preferably 1 min or more. More preferably it is in the range of 1 sec or more and 60 min or less, more preferably 5 sec or more and 30 min or less, even more preferably 10 sec or more and 15 min or less, still more preferably 30 sec or more and 15 min or less, still even more preferably 1 min or more and 5 min or less. However, as mentioned above, step (A) of the method and hence, also step (A-6) of the method may be performed multiple times (multiple performances).

Preferably, there is no upper limit for the duration, preferably the total duration, of the administration and/or application of conditioning measures, the conditioning energy and/or heat, and/or for the duration the of conditioning measures, conditioning energy and/or heat is administered and/or applied, preferably by using the skin-conditioning unit respectively the energizing means and/or heating means, as long as no burning lesions and/or damaging of skin like burning, causing lesions, rupture of capillaries and/or reflexive closure of the capillaries is caused. However, usually, the duration, preferably the total duration, is 48 hours or less, preferably 24 hours or less, even more preferably 12 hours or less, still more preferably 6 hours or less, still even more preferably 3 hours or less, further preferably 1 hour or less, even further preferably 30 min or less, still further preferably 15 min or less, still even further preferably 5 min or less. Usually, the duration, preferably the total duration, is 5 sec or more, more preferably 10 sec or more, even more preferably 30 sec or more, still more preferably 1 min or more. More preferably, the duration, preferably the total duration, is in the range of 5 sec or more and 48 hours or less, even more preferably 5 sec or more and 24 hours or less, still more preferably 10 sec or more and 12 hours or less, still even more preferably 10 sec or more and 6 hours or less, further preferably 10 sec or more and 3 hours or less, even further preferably 10 sec or more and 1 hour or less, still further preferably 10 sec or more and 30 min or less, still even further preferably 30 sec or more and 15 min or less, furthermore preferably 1 min or more and 5 min or less.

Preferably, the conditioning measures, respectively conditioning energy and/or heat, can be administered and/or applied continuously and/or pulsed and the skin-conditioning unit, the energizing means and/or heating means is configured respectively.

If not mentioned otherwise, it is to be understood that any embodiment or definition of the present invention described herein, particularly any embodiment or definition of the conditioning energy and/or the heat as described herein for step (A-6) of the method in terms of:
the temperature generated on the skin;
the amount of conditioning energy and/or heat delivered, administered and/or applied; within which time such amount is delivered, administered and/or applied;
the duration the conditioning energy and/or heat is administered and/or applied; and/or
the temperature of the energizing means and/or heating means,
is independently and mutatis mutandis applicable to the conditioning energy and/or the heat, respectively, as mentioned for the medical device, the skin-conditioning unit, the energizing means and/or heating means.

Preferably, in case the conditioning measures is provided by a negative pressure, the negative pressure is a pressure below an atmosphere pressure.

Preferably, in case the conditioning measures is provided by a negative pressure, the negative pressure is to an extent in the range of 0 hPa (hectopascale(s)) to 1,000 hPa, more preferably 0 hPa to 600 hPa.

Preferably, in case the conditioning measures is provided by a negative pressure, the negative pressure is 1,000 hPa or less, more preferably 600 hPa or less. Preferably, a lower limit for the negative pressure is 0 hPa, more preferably 10 hPa. More preferably, the negative pressure is in the range of 0 hPa to 1,000 hPa, more preferably 10 hPa to 600 hPa.

In any one of the embodiments of the present invention described herein, the expressions “temperature on the skin”, “temperature of the skin” and/or “skin surface temperature” may be used interchangeably. Similarly, in any one of the embodiments of the present invention described herein, the expressions “increased temperature on the skin”, “increased temperature of the skin” and/or “increased skin surface temperature” may be used interchangeably.

It is to be understood, that any one of the embodiments mentioned under the outline note ‘As further regards step (A-6) for any of the embodiments of the present invention described herein’ is independently applicable and/or combinable with any of the embodiments of the present invention described herein and particularly, with any embodiment of the medical device. Particularly, if not mentioned otherwise, it is to be understood that any embodiment or definition of the present invention described herein, particularly any embodiment or definition of the conditioning energy and/or the heat as described herein for step (A-6) of the method in terms of:
the temperature generated on the skin;
the amount of conditioning energy and/or heat delivered, administered and/or applied;
within which time such amount is delivered, administered and/or applied;
the duration the conditioning energy and/or heat is administered and/or applied; and/or
the temperature of the energizing means and/or heating means,
is independently and mutatis mutandis applicable to the conditioning measures, conditioning energy and/or the heat, respectively, as mentioned in respect of the medical device, the skin-conditioning unit, the energizing means and/or heating means.

Furthermore, if not mentioned otherwise, it is to be understood that any embodiment or definition of the present invention described herein, particularly any embodiment or definition of the energizing means and/or heating means described herein for step (A-6) of the method in terms of:
the temperature generated on the skin;
the amount of conditioning energy and/or heat delivered, administered and/or applied;
within which time such amount is delivered, administered and/or applied;
the duration the conditioning energy and/or heat is administered and/or applied; and/or
the temperature of the energizing means and/or heating means,
is independently and mutatis mutandis applicable to the medical devices, the medical device, the skin-conditioning unit, the energizing means and/or heating means.

Wherein the amount of the administered respective therapeutic agent, particularly in case it is an immunomodulatory substance(s) and/or the amount of conditioning measures, particularly in case it is conditioning energy or heat, is defined the same as mutatis mutandis defined in any of the embodiments of the present invention, particularly as administered in the method for treating and/or preventing as described herein particularly in steps (A), (A-6), (B), (B₁) and/or (C) of the method.

Preferably, the amount of the first therapeutic agent includes an immunomodulatory substance.

Advantageously, the medical device can enable a subject to perform at least some of the steps of any one of the embodiments and/or methods for treating and/or preventing an inflammatory disease, immunological disease and/or autoimmunological disease of a subject as described herein.

Advantageously, running the method for treating and/or preventing using the medical device can make it possible for a patient or a child to perform the therapeutic method easily and/or safely and/or age appropriate and/or by themselves, that can mean, without help of a physician, even if the patient has disabilities, particularly if the patient has disabilities when grasping. The easy and/or safely performance of the method for treating and/or preventing using the medical device is particularly given by the cooperative interaction of the skin-conditioning unit and the applicator unit.

Preferably, the skin-conditioning unit can be configured to administer conditioning measures to the skin, preferably for modifying a condition of the skin.

Preferably, the medical device additionally can include a coupling unit,
wherein particularly the coupling unit can be a part of the skin-conditioning unit; and
wherein the coupling unit is configured to couple the skin-conditioning unit and the applicator unit for the cooperative interaction of administering the first therapeutic agent with the conditioning measures; and
wherein particularly the coupling unit is configured to electrically, signally, wirelessly and/or mechanically couple the skin-conditioning unit and the applicator unit for the interactive coupling of the skin-conditioning unit and the applicator unit to cooperatively, and/or further preferably synergistically, administer the amount of the first therapeutic agent by means of the applicator unit with an amount of conditioning measures by means of the skin-conditioning unit to the skin of the subject.

Using other words, the cooperative interaction can be achieved by means of a coupling unit.

For the cooperative interaction, an order of the related steps is arbitrary, but the related steps have to be completely administered within a defined period of time and both have to be administered to an area of the skin, which is close enough that both administrations can, preferably synergistically, become effective within the skin.

Resulting from the cooperative interaction of the skin-conditioning unit and the applicator unit can be an associated administering of the conditioning measures and the administering of the respective therapeutic agent in such a form that both measures can interact for therapeutic effect. Preferably, the step of administering of the respective therapeutic agent is executed after the step of administering the amount of conditioning measures of the skin is completed.

Preferably, the skin-conditioning unit and the, particularly first, applicator unit, and/or a second applicator unit, and/or a third applicator unit respectively, can be configured to cooperatively interact to administer the amount of the first therapeutic agent, and/or an amount of a second therapeutic agent, and/or an amount of a third therapeutic agent respectively, with an amount of conditioning measures in respect to a coordinated administration of the respective amount of agents with the amount of conditioning measures, particularly within a defined period of time, particularly such that it is ensured that the amounts are administered to the respective areas of the skin.

Preferably, the medical device can include a control unit, which is configured to couple the skin-conditioning unit with the, particularly first, applicator unit, and/or a second applicator unit, and/or a third applicator unit respectively, for cooperative interaction of the skin-conditioning unit with the applicator unit, and/or the second applicator unit, and/or the third applicator unit.

Preferably, the medical device can comprise one release unit, wherein this one release unit comprises all units for running the method.

Alternatively or additionally, the medical device comprises a single release unit for triggering and/or driving the applicator unit; and one single release unit for triggering and/or driving the skin-conditioning unit.

Preferably, the medical device can include a control unit, which is configured to trigger an/or drive the first applicator, and/or the second applicator, and/or the third applicator respectively, for administering of the amount of first therapeutic agent, and/or second therapeutic agent, and/or third therapeutic agent respectively, after the amount of conditioning measures was administered by the skin-conditioning unit, for cooperative interaction of the skin-conditioning unit with the applicator unit, and/or the second applicator unit, and/or the third applicator unit respectively.

For cooperative interaction the order of the steps of administering the conditioning measures and the administering of the respective therapeutic agent are arbitrary, but it has to be assured that both administering is timely and locally sufficiently related, particularly for associated administering. Preferably, the step of administering of the respective therapeutic agent is executed after the step of administering the amount of conditioning measures of the skin is finalized. By this associated administering can be assured, because administering of the related therapeutic agent can be accomplished quickly, so that interruption of that step of the process seems unlikely.

Preferably, the skin-conditioning unit can be configured to include the coupling unit.

Preferably, the medical device can include a first reservoir, which is particularly a part of the applicator unit; and the first reservoir is configured for accommodation of the first therapeutic agent; and wherein the applicator unit is configured to administer the first therapeutic agent to the skin; and wherein the applicator unit is fluidly coupled to the first reservoir.

The applicator unit can include a first applicator to be configured to administer the first therapeutic agent to the skin. Therefore, the first applicator can be fluidly coupled to the first reservoir.

The first reservoir can include a separate reservoir and/or a reservoir, which is a member of the applicator unit. Particularly, the fluidly coupling of the applicator unit and the first reservoir can mean that the first therapeutic agent is attached to a surface of the applicator unit, particularly in such a way that the attached first therapeutic agent constitutes the fluidly coupled reservoir. Preferably, the first therapeutic agent can be attached to a surface of a means for applying the first therapeutic agent to the surface of the skin of the subject and/or to a layer of the skin of the subject. For instance, the, particularly first, applicator unit can include the means for applying the first therapeutic agent, which particularly, can be a cannula and/or a plurality of cannulas and/or the plurality of micro-cannulas, which is/are coated at its/their outside surface by the first therapeutic agent; and/or wherein the cannula and/or the plurality of cannulas and/or a plurality of micro-cannulas include the first therapeutic agent, particularly at an inside.

The, particularly first, reservoir can be configured to be exchangeable and/or to be refillable.

The medical device may be for single or repeated use, preferably for repeated use. Hence, medical device and/or the reservoir may be prefilled with the therapeutic agent, like for instance with an immunomodulatory substance, with a pharmaceutical composition, particularly with any of the pharmaceutical composition as mentioned herein.

Advantageously, using the medical device enables a user of the medical device to perform steps (B), (B₁) and/or step (C) of any one of the embodiments of the methods for treating and/or preventing an inflammatory disease, an immunological disease and/or an autoimmunological disease of a subject as described herein.

The first, second and/or third therapeutic agent can be an immunomodulatory substance, preferably a cytokine-like acting substance(s), more preferably an interferon-like acting substance(s), interleukin-like acting substance(s) and/or neurotrophin-like acting substance(s), even more preferably an IFN-γ-like acting substance(s), IL 4-like acting substance(s), BDNF-like acting substance(s) and/or IL-2 like acting substance(s), still even more preferably IFN-γ, IL 4, BDNF and/or IL-2.

The first therapeutic agent can be an immunomodulatory substance, preferably a first immunomodulatory substance.

The second therapeutic agent can be an immunomodulatory substance, preferably a second immunomodulatory substance.

The first therapeutic agent can be an immunomodulatory substance, preferably a second immunomodulatory substance.

According to an aspect, the skin-conditioning unit can be configured to administer conditioning measures to the skin, preferably for modifying a condition of the skin.

The conditioning measures can be administering conditioning energy to the, particularly surface, of the skin of the subject.

Administering conditioning measures can include an administering of a conditioning energy and/or administering of a skin-conditioning agent as already described above.

A conditioning energy can be energy, which can be administered to the skin of the subject for modifying the condition of the skin, wherein particularly the modification of the condition of the skin can be a step of any one of the methods and/or embodiments of the method for treating and/or preventing as described herein.

For administering the conditioning measures, and particularly the conditioning energy, to the skin, the medical device, and particularly the skin-conditioning unit, can include an energy source, which is electrically coupled to the skin-conditioning unit, for providing energy to administer conditioning measures to the skin by means of the skin-conditioning unit.

The energy source can include an electrical energy source, as for instance an electrochemical energy source; or preferably, an electrical battery; and/or a mechanical energy storage; and/or a reaction heat based energy source; and/or a latent heat source. The medical device can be configured to include an energy source, which is exchangeable, and/or to include an energy source, which is reconfigurable, as for instance a rechargeable electrical battery.

Advantageously, by using the medical device, the steps of any one of the methods for treating and/or preventing can be performed.

As stated above, administering of conditioning measures to the skin of the subject can, as an example, result in the following effects:
the vasodilation of the capillaries within the skin,
the increased blood volume within the skin,
the increased sO₂ and/or the increased rHb within the skin,
the increased temperature on the skin,
even more preferably,
the increased blood volume within the skin,
the increased sO₂ and/or the increased rHb within the skin, and/or
the increased temperature on the skin.

Preferably, the applicator unit can be configured to apply the first therapeutic agent to a first area of the skin of the subject; and wherein the skin-conditioning unit is configured to administer the amount of conditioning measures to the skin in a second area of the skin of the subject; and wherein the skin-conditioning unit is configured and/or arranged in respect to the applicator unit such, that, for associated administering the conditioning measures and the first therapeutic agent, the first area is close to, or at least partially overlapping, with the second area.

The associated administering can mean that the medical device is configured in such a way that the respective administering of the skin-conditioning unit and the applicator unit can support any one of the methods for treating and/or preventing an inflammatory disease, immunological disease and/or autoimmunological disease of a subject successfully.

Preferably, the second area of the skin corresponds to the skin area [a] as mentioned in the embodiments of the method described herein. If not mentioned otherwise, it is to be understood that any embodiment or definition described herein in terms of the skin area, particularly the skin area [a], is mutatis mutandis applicable to the second area of the skin as mentioned in any of the embodiments of the medical device described herein. Particularly, it is to be understood that any embodiment or definition described herein in terms of the skin area, particularly the skin area [a] regarding the size, shape, overlapping and/or the area sum is independently and mutatis mutandis applicable to the second area of the skin as mentioned in any of the embodiments of the medical device described herein.

More preferably, the second area of the skin corresponds to the application area [a] as mentioned in the embodiments of the method described herein. If not mentioned otherwise, it is to be understood that any embodiment or definition described herein in terms of the application area, particularly the application area [a], is mutatis mutandis applicable to the second area of the skin as mentioned in any of the embodiments of the medical device described herein. Particularly, it is to be understood that any embodiment or definition described herein in terms of the application area, particularly the application area [a] regarding the size, shape, overlapping and/or the area sum is independently and mutatis mutandis applicable to the second area of the skin as mentioned in any of the embodiments of the medical device described herein.

Preferably, the first area of the skin, the third area of the skin and the fourth area of the skin corresponds to the skin area [b], [b₁] and/or [c] as mentioned in the embodiments of the method described herein. If not mentioned otherwise, it is to be understood that any embodiment or definition described herein in terms of the skin area, particularly the skin area [b], [b₁] and/or [c], is independently and mutatis mutandis applicable to the first area of the skin, third area of the skin and/or forth area of the skin as mentioned in any of the embodiments of the medical device described herein. Particularly, it is to be understood that any embodiment or definition described herein in terms of the skin area, particularly the skin area [b], [b₁] and/or [c] regarding the size, shape, overlapping and/or the area sum is independently and mutatis mutandis applicable to first area of the skin, third area of the skin and/or forth area as mentioned in any of the embodiments of the medical device described herein.

More preferably, the first area of the skin, the third area of the skin and fourth area of the skin correspond to the skin area [b], [b₁] and/or [c] as mentioned in the embodiments of the method described herein. If not mentioned otherwise, it is to be understood that any embodiment or definition described herein in terms of the application area, particularly the application area [b], [b₁] and/or [c], is independently and mutatis mutandis applicable to the first area of the skin, third area of the skin and/or forth area of the skin as mentioned in any of the embodiments of the medical device described herein. Particularly, it is to be understood that any embodiment or definition described herein in terms of the application area, particularly the application area [b], [b₁] and/or [c] regarding the size, shape, overlapping and/or the area sum is independently and mutatis mutandis applicable to first area of the skin, third area of the skin and/or forth area as mentioned in any of the embodiments of the medical device described herein.

If not mentioned otherwise, it is to be understood that any of the embodiments or definitions described herein, particularly in any of the embodiments of the method and/or the medical use as described herein, is independently and mutatis mutandis applicable to the medical devices as mentioned in any of the embodiments described herein.

Particularly, if not mentioned otherwise, it is to be understood that any embodiment or definition described herein for the:
the immunomodulatory substance(s); the first immunomodulatory substance(s); the second immunomodulatory substance(s); the third immunomodulatory substance(s); the cytokine-like acting substance(s); the interferon-like acting substance(s); the interleukin-like acting substance(s); the neurotrophin-like acting substance(s); the IFN-γ-like acting substance(s); the IL-2-like acting substance(s); the IL-4-like acting substance(s); the BDNF-like acting substance(s); the cytokine(s); the interferon(s); the interleukin(s); the neutrophin(s); the IFN-γ; the IL-2; the IL-4; the BDNF; the amounts administered of the immunomodulatory substance(s) like the IFN-γ-like acting substance(s); the IL-2-like acting substance(s); the IL-4-like acting substance(s); the BDNF-like acting substance(s); the cytokine(s); the interferon(s); the interleukin(s); the neutrophin(s); the IFN-γ; the IL-2; the IL-4; the BDNF; the energizing means; the heating means; the conditioning energy; the heat; the skin-conditioning agent; the inflammatory disease, immunological disease and/or autoimmunological disease to be treated and/or prevented; the method for treating and/or preventing; the increased concentration of the substance(s) and skin-conditioning agent(s); the skin area; the application area; and/or the subject, particularly in terms of the method and/or the medical use, is independently and mutatis mutandis applicable to the medical device.

The second skin area can at least partially surround the first area and/or the second skin area can be operatively functional adjacent to the first skin area and/or the second skin area can be spatially functionally neighboring to the first area. A border of the second area can be spatially adjacent to a border of the first area.

The first skin area is related to the skin areas [b], [b₁] and/or [c], preferably the application areas [b], [b₁] and/or [c], depending on the therapeutic agent used in the method for treating and/or preventing an inflammatory disease, an immunological disease and/or an autoimmunological disease of a subject.

The second skin area is related to the skin area [a], preferably the application area [a], of the method for treating and/or preventing.

Using other words, the medical device can be configured to administer the first therapeutic agent to a different area of the skin than the skin-conditioning unit administers the conditioning measures, wherein both areas of the skin are, at least partially, adjacent to each other, or at least partially overlapping, in such a way that both areas are in a distance to each other, that the respective administering supports the medical method for successful medical treatment.

The medical device, which is configured to administer the conditioning measures to the second area of the skin and to administer the first therapeutic agent to a first area, wherein the first area and the second area can be different, can improve the use and/or the construction of the medical device. Particularly, the medical device can be configured such that the second area is arranged to, at least partially, enclose, the first area if the medical device is located at the surface of the skin of the subject.

The term skin is used to describe an affected area of the skin.

Preferably, as already discussed above, in any one of the methods and/or embodiments as described herein, in step (A) of the method the skin is of a skin area [a], in step (B) of the method the skin is of a skin area [b], in step (B₁) the skin is of a skin area [b₁] and/or in step (C) the skin is of a skin area [c].

For supporting any of the methods for treating and/or preventing as described above, the medical device is configured that the applicator unit can administer the first therapeutic agent to the first area of the skin, which is related to the skin area [b] of step (B) and/or to the skin area [b₁] in step (B₁) and/or to the skin area [c] in step (C).

Additionally or alternatively the medical device is configured that the skin-conditioning unit is configured to administer the conditioning measures to the second area of the skin of the subject, which is related to the skin area [a] of step (A).

Alternatively or additionally, in any one of the methods for treating and/or preventing and/or embodiments described herein, step (B), (B₁) and/or (C) are performed partially and/or totally within the same area of the skin where step (A) is performed at the medical device is configured accordingly.

The definition and/or relation of the skin areas mentioned here with respect to the medical device are the same as already described dealing with any one of the methods for treating and/or preventing an inflammatory disease, an immunological disease and/or an autoimmunological disease of a subject, as described herein.

The associated administering of the conditioning measures and the first therapeutic agent can mean that the first area of the skin and the second area of the skin are spatially and/or temporarily related in such a way, that there is a common area of the skin, where the conditioning measures administered to the second area of the skin and the administered therapeutic agent to the first area of the skin become effective. Particularly, the associated administering of the conditioning measures and the first therapeutic agent can mean, that the conditioning measures and the administering of the first therapeutic agent are administered in such a way and/or that the first area of the skin and the second area of the skin as defined by a configuration of the medical device can interact synergistically within the skin of the subject.

Preferably, the medical device can include a first applicator, which is particularly a member of the applicator unit; and wherein the first applicator is configured to be fluidly coupled with the first reservoir; and wherein the first applicator is configured for administering the first therapeutic agent to the first area of the skin.

Preferably, the medical device can include a first agent actuator, which is configured for providing the first therapeutic agent from the first reservoir to the first applicator, and preferably, the first agent actuator is configured to administer the first therapeutic agent to the skin of the subject.

Preferably, the medical device can include a second reservoir, which is configured to store a second therapeutic agent; and wherein the second reservoir is configured to be fluidly coupled to the first applicator, for administering the second therapeutic agent to the first skin area; and wherein the skin-conditioning unit is configured in respect to the first applicator such that, for associated application of the conditioning measures and the second therapeutic agent, the first area is close to, or at least partially overlapping, with the second area of the skin; and wherein the skin-conditioning unit and the first applicator are configured to cooperatively interact to administer the amount of the first therapeutic agent with an amount of the second therapeutic agent and with the amount of conditioning measures to the skin of the subject.

Advantageously, the medical device including a first and a second reservoir can support a medical method, which includes a step for administering a first therapeutic agent; and a step for administering a second therapeutic agent. The step for administering the second therapeutic agent can be step (B₁) or (C) of the method for treating and/or preventing an inflammatory disease, an immunological disease and/or an autoimmunological disease of a subject.

Preferably, the medical device can include a second reservoir, which is configured to accommodate a second therapeutic agent, which is particularly a member of the applicator unit; and a second applicator, which is particularly a member of the applicator unit; and wherein the second applicator is configured to be fluidly coupled with the second reservoir, and the second applicator is configured for administering the second therapeutic agent to a third area of the skin; and wherein the skin-conditioning unit is configured in respect to the first applicator and the second applicator such that, for associated application of the conditioning measures and the first therapeutic agent and the second therapeutic agent, the first area of the skin and the third area of the skin are close to, or at least partially overlapping, with the second area of the skin; and wherein the skin-conditioning unit and the first applicator and the second applicator are configured to cooperatively interact to administer the amount of the first therapeutic agent with an amount of the second therapeutic agent with the amount of conditioning measures.

Advantageously, by a plurality of reservoirs a plurality of therapeutic agents can be provided to the skin, for instance if the therapeutic plurality of agents are not stable if stored within one reservoir and/or if the plurality of agents are not mixable or not impermissible and/or if the plurality of agents if mixed together would need a specific medical approval for application and/or to prevent that the at least two of the plurality of substances interact during storing.

Preferably, the medical device can include a third reservoir, which is configured to accommodate a third therapeutic agent, which is particularly a member of the applicator unit; and a third applicator, which is particularly a member of the applicator unit; and wherein the third applicator is fluidly coupled with the third reservoir, and the third applicator is configured for directly contacting a fourth area of the skin for administering the third therapeutic agent; and wherein the skin-conditioning unit is configured in respect to the first applicator and the second applicator and the third applicator such that, for associated application of the conditioning measures and the first therapeutic agent and the second therapeutic agent and the third therapeutic agent, the first area of the skin and the third area of the skin and the fourth area of the skin are close to, or at least partially overlapping, with the second area of the skin; and wherein the skin-conditioning unit and the first applicator and the second applicator and the third applicator are configured to cooperatively interact to administer the amount of the first therapeutic agent with the amount of the second therapeutic agent with an amount of the third therapeutic agent with the amount of conditioning measures.

Preferably, the first applicator, and/or the second applicator, and/or the third applicator, can include a first needle injection unit, and/or a second needle injection unit, and/or a third needle injection unit respectively, to administer the first therapeutic agent, and/or second therapeutic agent, and/or second therapeutic agent respectively, to a layer of the first area of the skin, and/or third area of the skin, and/or forth area of the skin respectively.

The respective injection unit can include a respective syringe needle injection unit, and preferably, the medical device can be configured by means of the respective applicator that a depth of the injection of the skin by means of the respective needle injection unit and/or pressure injection unit is adjustable.

Alternatively or additionally, the respective needle injection unit can comprise a plurality of cannulas, particularly a number of 20 to 50 cannulas, which are configured particularly as a microneedle array.

Preferably, the first applicator, and/or the second applicator, and/or the third applicator can include a first topical employ unit, and/or second topical employ unit, and/or third topical employ unit respectively, to administer the first therapeutic agent, and/or the second therapeutic agent, and/or the third therapeutic agent respectively, to a layer of the first area of the skin, and/or third area of the skin, and/or forth area of the skin respectively, wherein the first therapeutic agent, and/or the second therapeutic agent, and/or the third therapeutic agent is formulated as a topical dosage.

Preferably, the first applicator and/or the second applicator and/or the third applicator comprises a first pressure injection unit, and/or second pressure injection unit, and/or third pressure injection unit respectively, to administer the first therapeutic agent, and/or second therapeutic agent, and/or third therapeutic agent respectively, to the first area of the skin, and/or third area of the skin, and/or a forth area of the skin respectively.

Advantageously, by a plurality of injection units a plurality of therapeutic agents can be provided to the skin by a plurality of applicators, for instance if the therapeutic plurality of agents are not stable if stored within one reservoir and/or if the plurality of agents are not mixable and/or if the plurality of agents if mixed together would need a specific medical approval for application.

Preferably, the respective applicator is configured such that the needle injection unit and/or the topical application unit and/or the pressure injection unit respective, are interchangeable, particularly for hygienic requirements to the device.

Preferably, the respective needle injection unit and/or pressure injection unit is configured to inject the amount of the respective therapeutic agent by the needle injection unit perpendicular to the respective area of the skin; and/or by the pressure injection unit perpendicular to the respective area of the skin; and preferably, to inject the amount of the respective therapeutic agent by the needle injection unit in an acute angel to the skin, wherein further preferably the acute angel includes 5 degree or more to 90 degree or less; and/or preferably, to inject the amount of the respective therapeutic agent by the pressure injection unit in an acute angel to the skin, wherein further preferably the acute angel includes 30 degree or more to 90 degree or less.

Preferably, the respective applicator unit and/or the respective applicator and/or the respective needle injection unit is configured to penetrate the skin to a selected depth, for injecting the respective therapeutic agent to a specific layer of the skin.

Preferably, the respective applicator unit and/or the respective applicator and/or the respective needle injection unit is configured to penetrate the skin of the subject into a selected depth within the skin, which is configurable.

Preferably, the respective applicator is configured by means of an respective applicator actuator to drive pressure injection unit and/or the needle injection unit to penetrate the skin of the subject to the selected depth, wherein particularly the respective applicator actuator is configurable in respect to a penetration depth of the pressure injection unit and/or the needle injection unit.

If the penetration depth is configurable, the medical device can be adapted to the need of different subjects operating the medical device, as for instance the thickness of the skin is different for different subjects.

Preferably, the respective applicator is configured to include the respective needle injection unit and/or topical employ unit and/or pressure injection unit; wherein particularly the respective needle injection unit and/or topical employ unit and/or pressure injection unit is configured to be exchangeable.

The exchange ability of the units as mentioned above enable to follow hygienic guidelines and easy way.

Preferably, the applicator unit and the skin-conditioning unit are configured, particularly enclosed by a common housing, as one integral unit.

Preferably, a form and/or a contour and/or dimensions of the housing for are configured for use of grasp-disabled people. For example, the housing can be formed bulky to enable the desktop use, and/or the housing can be formed with a waist, wherein the diameter of the waste is below and including 3 cm and/or the form of the housing can take into account a use of the subject which is disabled to close the fingers and/or which is disabled to form a first.

Preferably, the administering of the conditioning measures comprises:
administering a conditioning energy; and/or
administering a skin-conditioning agent.

Advantageously, by this the medical device can be adapted to specific needs and/or the different subjects operating the medical device.

Preferably, the administering the conditioning energy, can mean an administering of an amount of the conditioning energy to the second area of the skin; and/or an administering of an amount of a topical dosage to the second area of the skin, which comprises an administering of a skin-conditioning agent.

Advantageously, by this the medical device can be adapted to specific needs.

Preferably, the skin-conditioning unit can be a heat source unit, which is configured to contact the second area of the skin; and configured to transfer heat to the second area of the skin, for administering conditioning measures; and/or
an electromagnetic radiation source unit, preferably an infrared (IR)-light source unit, which is configured to provide light, respectively IR-light, to the second area; and configured to transfer the respective light to the second area of the skin, for administering conditioning measures: and/or a particularly mechanical, skin agitation unit, which is configured to contact the second area of the skin for skin agitation; and configured to transfer mechanically agitation to the second area of the skin, for administering conditioning measures.

Advantageously, by use of the different skin-conditioning units the medical device can be adapted to specific needs of the subject.

Particularly, if the medical device is configured to be adaptable in respect to different skin-conditioning units, which can be exchanged with each other.

Preferably, the skin-conditioning unit particularly a heating unit, of the medical device is configured to be exchangeable.

By means of an exchangeable heating unit, the administering of the conditioning measures can be adapted to specific needs by using different heating units, which are administered different amounts of conditioning measures.

Preferably, the medical device can include a contact detection means for monitoring a proximity of the skin-conditioning unit with the skin;
for controlling a modification of the condition of the skin; and/or
for monitoring the proximity of the applicator unit with the skin, for controlling the administering of the first therapeutic agent and/or the conditioning measures to the skin of the subject.

Advantageously, the contact detection means can enable the medical device to control a correct and safe operation of the medical device.

The contact detection means can verify and/or monitor and/or detect a readiness of the medical device for injecting the related therapeutic agent. Other sensor devices can monitor the penetration of an injection needle into the skin and control the execution of the injected amount into the skin.

The contact detection means can be located within the base plate of the medical device for detecting a proximity of the skin of the subject.

The medical device can be configured, to generate a signal if the medical device is correctly placed on the skin. Safe and correct penetration of the injection needle into the skin can be preferably performed by means of an impedance measurement, determining a value for the impedance between the needle and a surface of the skin. Alternatively or additionally, an injection of the needle into the skin can be controlled by an infrared (IR) sensor.

Preferably, a functionality of the contact detection means is based on
an electrical detection principle; and/or on an impedance detection means; and/or
on a capacitive detection means; and/or on a mechanical detection principle.

This control of the contact of the medical device with the skin can increase safety handling of the medical device during use.

Preferably, the medical device can include a temperature sensor for determining an administered amount of conditioning measures to the skin, by measuring the surface temperature of the skin; and wherein particularly the skin-conditioning unit comprises the temperature sensor;

Advantageously the temperature sensor can enable the cooperatively interaction of the skin-conditioning unit and the applicator unit, because particularly the amount of conditioning measures can be detected for verifying a complete administering of the conditioning measures to the skin.

Preferably, the medical device can include a second agent actuator, configured to provide the second therapeutic agent from the second reservoir to the second applicator; and/or a third agent actuator, configured to provide the third therapeutic agent from the third reservoir to the third applicator.

Particularly, if the actuator s electrically driven, the use of the medical device is advantageously simple and easy.

Advantageously, by a plurality of reservoirs a plurality of therapeutic agents can be provided to the skin by a plurality of applicators, for instance if the therapeutic plurality of agents are not stable if stored within one reservoir and/or if the plurality of agents are not mixable and/or if the plurality of agents if mixed together would need a specific medical approval for application.

Preferably, the first and/or the second and/or the third actuator can be the same actuator.

Particularly, the actuator can be manual operated actuator.

Preferably, the medical device can include a first dosage unit, and/or a second dosage unit, and/or a third dosage unit, wherein the first dosage unit, and/or the second dosage unit, and/or the third dosage unit is coupled to the first applicator, and/or the second applicator, and/or the third applicator respectively; and/or wherein particularly the first dosage unit, and/or the second dosage unit, and/or the third dosage unit is coupled to a first agent actuator, and/or a second agent actuator and/or a third agent actuator respectively; and/or wherein the first dosage unit, and/or the second dosage unit, and/or the third dosage unit is coupled to the first reservoir, and/or the second reservoir, and/or the third reservoir respectively, wherein the respective dosage unit is configured for administering a respectively specified amount of the first therapeutic agent and/or the second therapeutic agent and/or the third therapeutic agent to the respective area of the skin.

Preferably, the respective applicator unit can be configured to administer a specified amount of the respective therapeutic agent to the skin, wherein the specified amount of the respective therapeutic agent is configurable, particularly by means of the dosage unit.

Preferably, the medical device can be configured, particularly by means of the respective dosage unit; to configure a specified amount of the respective therapeutic agent; and/or wherein the medical device is configured, particularly by means of the respective applicator, to configure a depth of a penetration of the skin of the subject, by means of the respective needle injection unit and/or the respective pressure injection unit; and/or wherein the medical device is configured to configure a specified amount for administering the conditioning measures.

Advantageously, the configuration of the respective amounts enables to configure the medical device to adapt and/or specify the method for treating and/or preventing in respect to needs of specific subjects.

Preferably, the medical device is configured to store, within a storage area of the medical device, a specified depth of penetration of the skin of the subject, by means of the respective needle injection unit and/or a pressure injection unit; and/or to store, within the storage area of the medical device, a specified amount of the respective therapeutic agent, which is administered each time the medical device is used; and/or wherein preferably the medical device is configured to protect an access to the storage area for modification of respective stored data to authorized subjects only.

Advantageously, and access of the storage area can be restricted to specific to specific users of the external devices, e.g. by means of a secret key and/or by a restrict access, to make sure that the correct specified amounts are provided by the medical device.

Preferably, the medical device can include a means for wireless coupling of the medical device with an external device, for enabling a signal coupling, particularly based on a Bluetooth standard, to configure the specified amount for administering the respective therapeutic agent; and/or to configure a specified amount for administering the conditioning measures; and/or to configure the respective specified depth of a penetration of the skin of the subject, by means of the respective needle injection unit and/or the respective pressure injection unit, using the external device.

Advantageously, by this access to the medical device for changing the specified amounts can be exclusive for physicians.

Preferably, the means for wireless coupling can be configured to give access by means of an external device via the wireless coupling, which is restricted to specific external devices and/or to specific users of the external devices.

Preferably, the medical device can be configured such that the first reservoir, and/or the second reservoir, and/or the third reservoir are replaceable; and preferably that the first reservoir, and/or the second reservoir, and/or the third reservoir is embodied as a capule or a cartridge or a syringe or an ampule or vial or an injectable dosage form, preferably the injectable dosage form as mentioned in any of the embodiments described herein.

This enables the medical device to store different injectable dosage forms, which are already described in the specification.

Preferably, the first reservoir, and/or the second reservoir, and/or the third reservoir and accordingly the medical device can be configured to be refillable in respect to the respective therapeutic agent.

Advantageously, a refillable respective reservoir enables a subject to be provided with the needed pharmaceutical in an amount, which can be stored without deterioration. That is particularly important if the used therapeutic agent has a poor stability. Alternatively or additionally, the medical device can be configured to refill the respective reservoir by use of an electrical driven syringe of the medical device.

Preferably, the first reservoir, and/or the second reservoir, and/or the third reservoir and accordingly the device can be configured to be a multi-compartment type of a capule, and/or of a cartridge, and/or of a syringe, and/or of an ampule, and/or of a vial, and/or of an injectable dosage form, preferably the injectable dosage form as mentioned in any of the embodiments described herein, for separate storing of component of the respective therapeutic agent the respective.

Such a multi-component type of the respective reservoir can be configured to include a fluid, particularly water, which can be given to a pulverized substance, wherein particularly the pulverized substance is in a lyophilisate form, such that the pulverized substance can be dissolved in solvent to solve or reconstitute the lyophilisate form of the substance by use of a pharmaceutical acceptable solvent.

Using other words the capule, and/or of a cartridge, and/or of a syringe, and/or of an ampule, and/or of a vial, and/or of an injectable dosage form, preferably the injectable dosage form as mentioned in any of the embodiments described herein, can be configured to be in a two chambers configuration. Wherein the medical device is configured to be suitable to operate with at least one of the different types of the respective reservoir.

Preferably, the medical device can include an energy source, which can be at least coupled to the skin-conditioning unit, for providing conditioning energy to generate and/or administer conditioning measures to the second area of the skin by means of the skin-conditioning unit.

The energy source can be an electrical energy source for driving the different submodules of the medical device. Alternatively or additionally, the energy source can be based on electrical energy and/or reaction heat and/or latent heat and/or electrochemical energy and/or start mechanical energy, preferably by means of the spring, which is configured to be preloaded. Advantageously, the energy source can be used for driving the actuator and/or for administering of the respective therapeutic agents.

Preferably, the medical device can include a signal generator, which is configured to generate a signal at the beginning of the cooperative interaction of the skin-conditioning unit and the applicator unit, and/or during the cooperative interaction of the skin-conditioning unit and the applicator unit, and/or at the end of the cooperative interaction of the skin-conditioning unit and the applicator unit, preferably for controlling a use of the device for administering a therapeutic agent to a first area of a skin.

For application of the medical device, the user gets feedback on correct use and/or a faulty performance of the medical device.

If the signal generator displays the signal using a user interface, as for instance a bargraph, number of bars at the bargraph can inform the user of the medical device about the progress performing the steps of the method for treating and/or preventing. Alternatively or additionally, the signal generator can display a countdown of the course of the medical method.

Preferably, the signal generator can be configured to generate an optical signal, and/or an acoustical signal, and/or a wireless signal.

Advantageously, signals provided by the medical device can increase safe and correct handling of the medical device by a subject.

Preferably, the signal generator can be configured to generate the signal if the contact detection means according as described above detects a loose proximity of the skin-conditioning unit with the skin; and/or wherein the signal generator is configured to generate the signal if the contact detection means according as described above detects a loose proximity of the applicator unit with the skin.

Preferably, the contact detection means can be located in a base plate of the medical device, were also the skin-conditioning unit and/or the applicator unit is located to come into contact with the skin.

Alternatively or additionally, the signal generator can be configured to generate the signal if the steps of the method are performed correctly and/or an error occurred.

Preferably, the medical device can include a trigger that can be operated from outside the device, for activating a cooperatively interacted administration of the amount of first therapeutic agent, and/or second therapeutic agent, and/or third therapeutic agent respectively, with an amount of conditioning measures by means of the device.

For instance this can be a power on switch, which enables the medical device to start with the cooperative interaction if the contact detection means verify that the medical device is handled correctly on top of the surface of the skin.

Preferably, the medical device can include a marker unit for application of a preferably removable mark on any of the skin areas preferably the second skin area, for therapeutic use.

If there is an interruption or break during the performance of the method for treating and/or preventing the resulting removable marks can be used to continue the method by executing the remaining steps and/or time and/or parts of the method at the same area of the skin. Alternatively, with the resulting removable marks the method can be continued also with another device. Alternatively or additionally, by using the removable marks, the method can be repeated and/or parts of the method can be repeated at the same area of the skin using the medical device.

Preferably, the medical device can include a radiation source, which is configured to impact the needle injection unit and/or the pressure injection unit and/or the topical dosage unit, for sterilization of the units, wherein preferably the radiation source is the source for ultraviolet (UV) light.

Advantageously, the medical device with the radiation source for sterilization can improve the sterilization state of the medical device for medical use.

Preferably, the first therapeutic agent, and/or second therapeutic agent, and/or the third therapeutic agent can be an immunomodulatory substance(s), wherein particularly the first therapeutic agent and the second therapeutic agent and the third therapeutic agent are different therapeutic agents, preferably different immunomodulatory substance(s).

According to an aspect, the medical device can be configured such that the skin-conditioning unit of the medical device can be configured to be detachable from the applicator unit, and wherein the skin-conditioning unit can be configured to operate independently from the applicator unit.

This configuration of the medical device allows the skin-conditioning unit of the medical device to be spatially separated from the applicator unit by mechanically and/or electrically decoupling the skin-conditioning unit from the application unit, for administering conditioning measures to the skin by use of the skin-conditioning unit, which is separated.

Advantageously, the resulting different parts of medical device can be used separately and/or the resulting different parts can be stored in different environments to optimize the storage.

A medical device is proposed, which can be configured to be suitable for use in a method for treating and/or preventing an inflammatory disease, immunological disease and/or autoimmunological disease in a subject.

Advantageously, if the medical device to perform a suitable for use in the method for treating and/or preventing enables a subject can perform the method in an easy and safe way.

As already stated above, a kit of parts (V) is proposed, which can include
an applicator unit; and
a skin-conditioning unit; and
optionally a reservoir, comprising a immunomodulatory substance.

Preferably, the reservoir is configured to be fluidly coupled to the applicator unit; and wherein the skin-conditioning and/or the applicator unit is configured to be mechanically and/or electrically coupled to cooperatively interact to administer to a skin of a subject an amount of a first therapeutic agent by means of the applicator unit with an amount of conditioning measures by means of the skin-conditioning unit.

Advantageously, the kit of parts enables a subject to use the medical device directly after the medical device is handed over to the subject, because the respective therapeutic agent can be stored in a different way than the rest of the medical device to optimize the storage conditions for the different components of the medical device. This can be particularly important because of deterioration of the respective therapeutic agent and/or because of vulnerable sterile conditions, for instance of injection needles and/or to take into account optimal conditions to store an energy source like for instance a battery or accumulator. For instance, the applicator unit, which may comprise an energy source like a battery may be recharged or may protected from coldness, the skin-conditioning unit comprising the therapeutic agent can be stored separately in a cool place.

Advantageously, the medical device is configured such that the skin-conditioning unit of the medical device can be configured to be detachable from the applicator unit as described above, to store the different units according to an optimal condition of the environment of the storage.

Preferably, the kit of parts include a further reservoir, comprising a further immunomodulatory substance.

Preferably, the kit of parts further include an applicator, wherein preferably the applicator comprises a syringe and/or an injection needle and/or a microneedle array.

If not mentioned otherwise, it is to be understood that any embodiment or definition of the applicator unit, the skin-conditioning unit, the reservoir and/or the immunomodulatory substance described herein in any of the embodiments of the medical device is independently and mutatis mutandis applicable to the applicator unit, the skin-conditioning unit, the reservoir and/or the immunomodulatory substance as mentioned for the kit of parts (V).

Preferably, the medical device and/or the kit of parts (V), which is suitable for use in the method for treating and/or preventing an inflammatory disease, immunological disease and/or autoimmunological disease in a subject is suitable to perform the following steps of the method:
a step (A), wherein the step (A) is selected from one or more of the following steps, wherein at least one of the steps is executed by means of a skin-conditioning unit of the medical device and/or of the kits of parts (V):
(A-0) generating an accumulation of PBMCs within the skin of the subject;
(A-1) generating a vasodilation of the capillaries within the skin of the subject;
(A-2) generating an increased blood volume within the skin of the subject;
(A-3) generating an increased sO₂ within the skin and/or an increased rHb within the skin of the subject;
(A-4) generating an increased temperature on the skin of the subject;
(A-5) generating a redness on the skin of the subject;
(A-6) administering energy to the skin of the subject;
(A-7) administering a skin-conditioning agent to the skin of the subject; and/or
(A-8) administering PBMCs into the skin of the subject,
and the method further comprises the following step(s), and wherein at least one of the steps is executed by means of an applicator unit of the medical device of the medical device and/or of the kits of parts (V):
(B) administering the immunomodulatory substance(s) to the skin of the subject; and/or
(C) administering the immunomodulatory substance(s) to the skin of the subject, wherein immunomodulatory substance(s) administered in step (C) is different from the immunomodulatory substance(s) administered in step (B).

A use of any one of the medical devices and/or any of the kit of parts (V) as described herein is proposed in a method for treating and/or preventing an inflammatory disease, immunological disease and/or autoimmunological disease in a subject, comprising
a step (A), wherein the step (A) is selected from one or more of the following steps, wherein at least one of the steps is executed by means of a skin-conditioning unit of the medical device and/or of the kits of parts (V):
(A-0) generating an accumulation of PBMCs within the skin of the subject;
(A-1) generating a vasodilation of the capillaries within the skin of the subject;
(A-2) generating an increased blood volume within the skin of the subject;
(A-3) generating an increased sO₂ within the skin and/or an increased rHb within the skin of the subject;
(A-4) generating an increased temperature on the skin of the subject;
(A-5) generating a redness on the skin of the subject;
(A-6) administering energy to the skin of the subject;
(A-7) administering a skin-conditioning agent to the skin of the subject; and/or
(A-8) administering PBMCs into the skin of the subject,
and the method further comprises the following step(s), and wherein at least one of the steps is executed by means of an applicator unit of the medical device and/or of the kits of parts (V):
(B) administering the immunomodulatory substance(s) to the skin of the subject; and/or
(C) administering the immunomodulatory substance(s) to the skin of the subject,
wherein immunomodulatory substance(s) administered in step (C) is different from the immunomodulatory substance(s) administered in step (B)

Advantageously, using the medical device and/or the kit of parts (V) to perform the method for treating and/or preventing enables a subject an easy and safe conducting of the method.

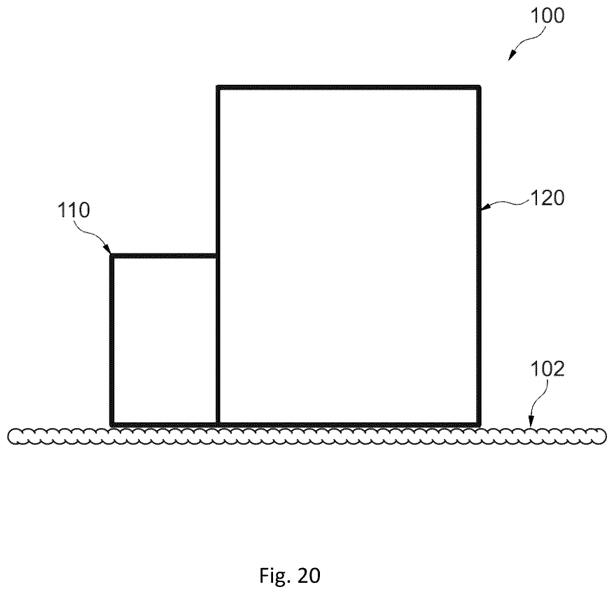

FIG. 20 sketches schematically a block diagram of a first embodiment of a medical device 100 having a skin-conditioning unit 110 and an applicator unit 120. The skin-conditioning unit 110 and the applicator unit 120 are configured, for instance by a cooperatively interacting coupling of the skin-conditioning unit 110 and the applicator unit 120, to act to a skin 102 of a subject.

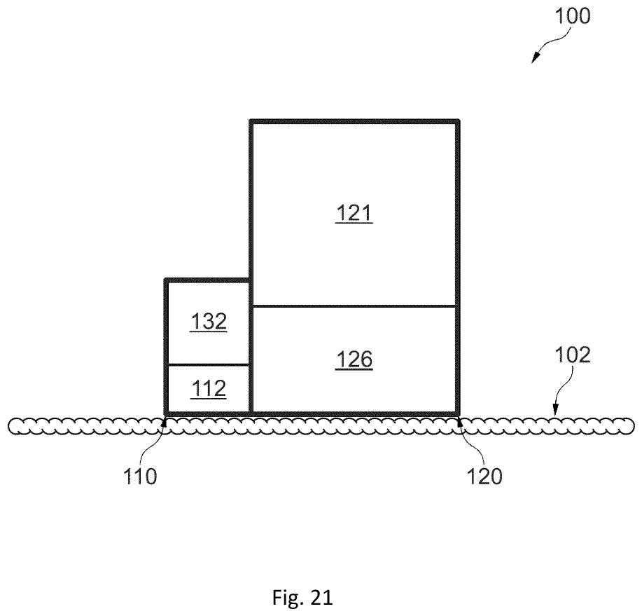

FIG. 21 sketches schematically a block diagram of a second embodiment of the medical device 100 including the skin-conditioning unit 110 and the applicator unit 120.

The skin-conditioning unit 110 and the applicator unit 120 are configured to cooperatively interact, by means of a control unit 132, being part of the skin-conditioning unit 110, to administer an amount of a first therapeutic agent, which is stored in a first reservoir 121 of the applicator unit 120, by means of the first applicator 126 with an amount of conditioning measures by means of a heat source unit 112, being part of the skin conditioning unit 110, to the skin 102. Therefore, the control unit 132 is coupled to the heat source unit 112 and the applicator unit 120 and the first applicator 126. The first reservoir 121 is fluidly coupled to the first applicator 126.

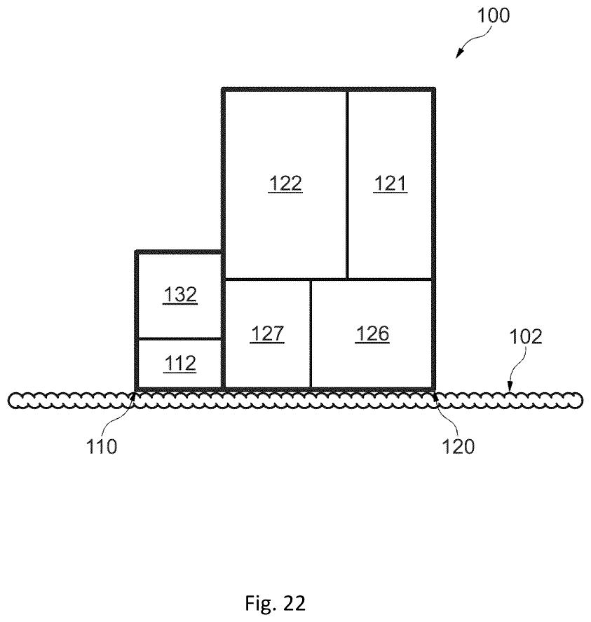

FIG. 22 sketches schematically a block diagram of a third embodiment of a medical device 100 having a skin-conditioning unit 110 and an applicator unit 120. The skin-conditioning unit 110 and the applicator unit 120 are configured to interact cooperatively, by means of the control unit 132, to administer an amount of a first therapeutic agent, which is stored within the first reservoir 121, by means of the first applicator 126 with an amount of conditioning measures by means of the heat source unit 112 of the skin-conditioning unit 110. The control unit 132 is coupled to the heat source unit 112 and the applicator unit 120 and the applicator actuator 127, which is coupled to the first applicator 126 for actuation of the first applicator 126.

The first reservoir 121 of the medical device 100 is configured to store the first therapeutic agent. The first reservoir 121 is coupled to an actuator 122 of the first reservoir 121, wherein the actuator 122 is coupled to the control unit 132 for controlling a provision of the first therapeutic agent to the first applicator 126. Moreover, the applicator unit 120 is configured to administer the first therapeutic agent to the skin 102 by means of the first applicator 126, wherein the first applicator 126 of the applicator unit 120 is fluidly coupled to the first reservoir 121 of the applicator unit 120. The first applicator 126 is coupled to an applicator actuator 127, wherein the applicator actuator 127 is configured to actuate the first applicator 126 for administering the first therapeutic agent to the skin.

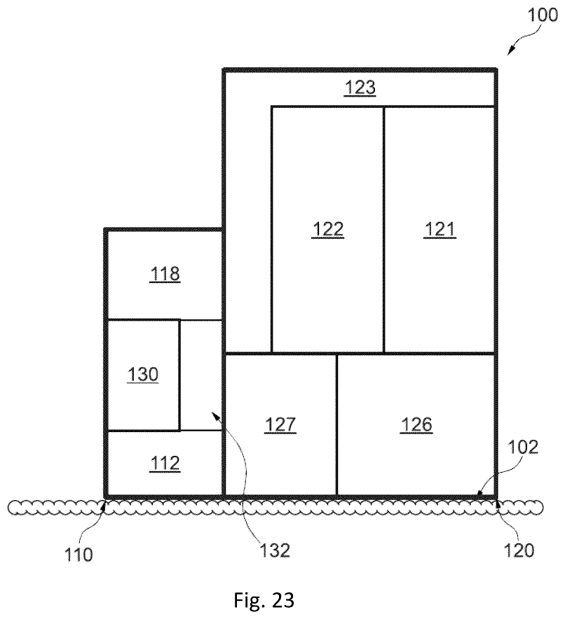

FIG. 23 sketches schematically a block diagram of a fourth embodiment of a medical device 100 including a skin-conditioning unit 110, which is configured to apply conditioning measures to the skin 102, preferably for modifying a condition of the skin 102, and an applicator unit 120.

The skin-conditioning unit 110 and the applicator unit 120 are configured to interact cooperatively, by means of the control unit 132, to administer an amount of a first therapeutic agent, which is stored within a first reservoir 121, by means of a first applicator 126 with an amount of conditioning measures by means of a heat source unit 112 of the skin-conditioning unit 110. The heat source unit 112 is configured to provide heat to a second area of the skin 102.

The control unit 132 is coupled and can be controlled by a controller 130 of the medical device 100. The control unit 132 is coupled to the heat source unit 112 and the applicator unit 120 and to the applicator actuator 127, which is coupled to the first applicator 126, and to the dosage unit 123, for cooperative interaction of the skin-conditioning unit and the applicator unit to administer the first therapeutic agent to a skin 102 of a subject an amount of a first therapeutic agent by means of the applicator unit 120 with an amount of conditioning measures by means of the skin-conditioning unit 110.

The control unit 132 is coupled to the actuator 122 by means of a dosage unit 123, which is configured to control the amount of the first therapeutic agent administered to the skin. Therefore, the dosage unit 123 is coupled to the actuator 122 of the first reservoir 121 and can be coupled to the first reservoir 121, wherein the actuator 122 is coupled to the control unit 132 for controlling a provision of the first therapeutic agent to the first applicator 126.

An energy source 118, which can be part of the skin-conditioning unit 110, is coupled to the heat source unit 112, wherein the energy source 118 is configured to provide energy for generating heat by means of the heat source unit 112 and/or for providing energy to drive the control unit 132 and/or the controller 130. The energy source 118 can also be coupled to the actuator 122 and to the applicator actuator 127 and to a dosage unit 123 for providing conditioning energy.

The first reservoir 121 of the medical device 100 is configured to store the first therapeutic agent. The first reservoir 121 is coupled to the actuator 122, wherein the actuator 122 is coupled to the control unit 132, via the dosage unit 123, for controlling a provision of the first therapeutic agent to the first applicator 126. Moreover, the applicator unit 120 is configured to administer the first therapeutic agent to the skin 102 by means of the first applicator 126, wherein the first applicator 126 of the applicator unit 120 is fluidly coupled to the first reservoir 121 of the applicator unit 120. The first applicator 126 is coupled to an applicator actuator 127, wherein the applicator actuator 127 is configured to actuate the first applicator 126 for administering the first therapeutic agent to a first area of the skin. The applicator actuator 127 is coupled to the control unit 132 for controlling the administering of the first therapeutic agent to the skin of the subject.

Moreover, the skin-conditioning unit 110, by means of the heating unit 112, is configured in respect to the applicator unit 120, by means of the first applicator 126, such, that, for associated administering of the conditioning measures and the first therapeutic agent, the first area is close to, or at least partially overlapping, with the second area.

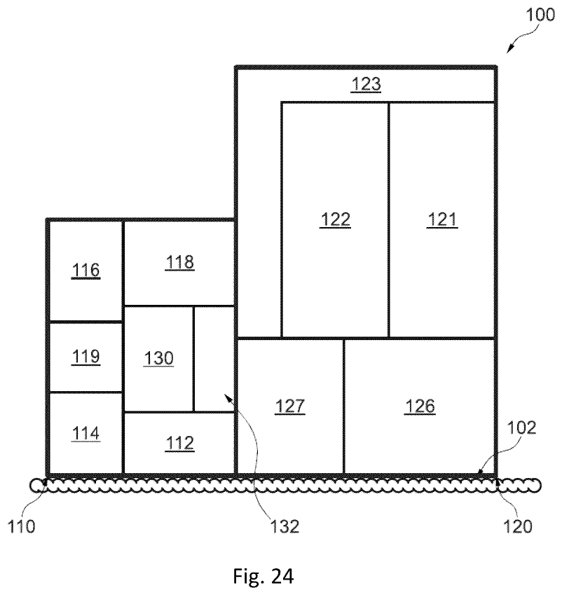

FIG. 24 sketches schematically a block diagram of a fifth embodiment of a medical device 100, which corresponds to the fourth embodiment, which is shown and described with respect to FIG. 23.

In addition to the fourth, the fifth embodiment sketches schematically a contact detection means 114, being part of the skin-conditioning unit 110, for monitoring of the proximity of the skin-conditioning unit 110 with the surface of the skin 102, which is coupled to the controller 130. By this, the controller 130 can control as well a cooperative interaction of the skin-conditioning unit and the applicator unit to administer the first therapeutic agent to a skin 102 of a subject, as also a correct handling of the medical device 100 during the cooperative interaction. Additionally, the fifth embodiment of the medical device 100 includes a trigger 119, wherein the trigger 119 is configured to start the cooperative interaction of the skin-conditioning unit 110 and the applicator unit 120 to administer to a skin of a subject an amount of the first therapeutic agent with an amount of conditioning measures, wherein the conditioning measures in this case is an administration of heat to the skin by means of the heating unit 112. Therefore, the trigger 119 is coupled to the controller 130, wherein the controller 130 is coupled to the control unit 132. Additionally, the trigger 119 can be coupled to the contact detection means 114, particularly for interrupting the cooperative interaction in case the medical device 100 is not handled correctly by a subject.

The fifth embodiment of the medical device 100 can also include a wireless coupling means 116, which is coupled to the controller 130, for enabling a signal coupling with an external device, to configure a specified depth of penetration of the skin 102 of the subject by the first applicator 126, which can include the needle injection unit, and/or to configure a specified amount for administering the first therapeutic agent to the skin 102 and all to configure a specified amount of conditioning measures to the skin 102.

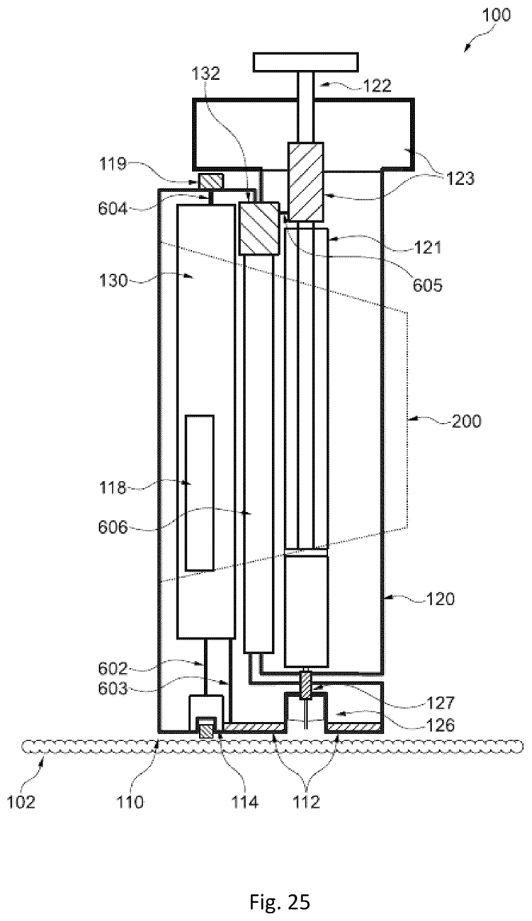

FIG. 25 schematically sketches a medical device 100 as a sixth embodiment.

The medical device 100 includes a skin-conditioning unit 110, which can be brought in direct contact with a skin 102 of a subject by means of the heating unit 112, and includes an applicator unit 120.

The heating unit 112 is in contact with a second area of the skin 102 if the medical device 100 is in an operational position to apply conditioning measures in form of heat to the skin 102. The second area of the skin 102 is defined here by the contact area between the heating unit 112 and the skin 102.

The heating unit 112 is configured to apply conditioning measures in form of heat to the skin 102, preferably for modifying a condition of the skin 102.

The skin-conditioning unit 110 is provided by a handle 200 to operate the medical device 100 by a subject.

The skin-conditioning unit 110 is configured to monitor a direct contact of the heating unit 112 with the surface of the skin by means of a contact detection means 114, which is signally coupled 602 to a controller 130 of the skin-conditioning unit 110 for monitoring a proximity of the skin-conditioning unit 110 with the skin 102.

The contact detection means 114 can be implemented using the functionality of an electrical switch, where the switch-operating lever of the electrical switch is configured and located at the skin-conditioning unit 110 to be triggered if the skin-conditioning unit 110 is correctly contacting the surface of the skin 102 by means of the heating unit 112.

The skin-conditioning unit 110 is mechanically coupled to the applicator unit 120 by a slide bearing connection 606, which enables the applicator unit 120 to be moved into a direction perpendicular to the skin 102 if the medical device 100 is in an operational position on top of the surface of the skin 102, which means, that the skin-conditioning unit 110 and an applicator unit 120 are configured to be moved in respect to each other. Particularly if the skin-conditioning unit 110 is placed on the surface of the skin by means of the heating unit 112, the applicator unit 120 can be moved closer to the surface of the skin, which can be controlled by the control unit 132 and/or
the dosage unit 123.

The skin-conditioning unit 110 and the applicator unit 120 are configured to interact cooperatively, by means of the control unit 132, to administer an amount of a first therapeutic agent, which is stored within the first reservoir 121, by means of the first applicator 126 with an amount of conditioning measures by means of the heat source unit 112 of the skin-conditioning unit 110.

For this cooperative interaction the medical device 100 is configured such that by manual pushing the actuator 122 the applicator unit 120 is moved relative to the skin-conditioning unit 110 to come in contact with the trigger 119, which is configured as an electrical switch, and wherein the trigger 119 is electrically coupled 604 to the controller 130, to start the administering of heat to the skin 102 by means of the heating unit 112. The applicator 122 can be configured as a rot with a handling knob at one end, and wherein the rot operates a piston, which is located within a cylinder, wherein the cylinder wall is configured to form the first reservoir. Moving of the rot as moving of the applicator 122, within the cylinder will push out the first therapeutic agent from the first reservoir 121.

The heating unit 112 is electrically coupled 603 to the controller 130 and to the energy source 118 to provide electrical energy to the heating unit 112. If a temperature sensor placed adjacent to the surface of the skin 102, which is not shown here, detects a selected increase of the temperature of the surface of the skin 102 and/or if the controller 130 generates a release signal to the coupling unit 132 after a specified amount of time of operating the heating unit 112 is elapsed, the sliding connection 606 is further released such that the first applicator 126 can penetrate the skin 102 by means of a syringe needle of the first applicator 126. The coupling 605 between the coupling unit 132 and the dosage unit 123 is configured to release the applicator even further if the skin 102 is penetrated, to enable by means of a related release of the actuator 122 caused by the dosage unit 123 to provide the first therapeutic agent, which is stored within the first reservoir 121, to the first applicator 126, by means of fluidly coupling 127 of the first applicator 126 and the first reservoir 121. Because of the fluidly coupling 127, the first therapeutic agent can be administered to the skin of the subject.

By means of this described configuration of the medical device 100 the control unit 132 couples the heat source unit 112 and the applicator unit 120 and the applicator actuator 127, which is coupled to the first applicator 126, and the dosage unit 123, for cooperative interaction of the skin-conditioning unit and the applicator unit to administer the first therapeutic agent to a skin 102 of a subject an amount of a first therapeutic agent by means of the applicator unit 120 with an amount of conditioning measures by means of the skin-conditioning unit 110.

Further, by this described configuration the control unit 132 is coupled to the actuator 122 by means of the dosage unit 123, which is configured to control the amount of the first therapeutic agent administered to the skin. As described, the dosage unit 123 is coupled to the actuator 122 of the first reservoir 121 and mechanically couple's to the first reservoir 121 by limiting the movement of the actuator 122, which is particularly implemented as a rot, which can be pushed downward into the direction of the skin 102.

Using other words, the first reservoir 121 of the medical device 100 is configured to store the first therapeutic agent. The first reservoir 121 is coupled to an actuator 122, wherein the actuator 122 is coupled to the control unit 132, via the dosage unit 123, for controlling a provision of the first therapeutic agent to the first applicator 126.

Moreover, the applicator unit 120 is configured to administer the first therapeutic agent to the skin 102 by means of the first applicator 126, wherein the first applicator 126 of the applicator unit 120 is fluidly coupled to the first reservoir 121 of the applicator unit 120.

The first applicator 126 is coupled to an applicator actuator 127, wherein the applicator actuator 127 is configured to actuate the first applicator 126 for administering the first therapeutic agent to a first area of the skin.

The actuator 122 is mechanically coupled to an applicator actuator 127, which is coupled to the control unit 132 for controlling the administering of the first therapeutic agent to the skin of the subject in form of a movement of the first applicator 126 for penetrating the skin 102 by means of a needle injector, which is part of the applicator 126.

For associated administering of the conditioning measures and the first therapeutic agent, the first area, which is defined by a vicinity of the first applicator 126 and/or by a vicinity of the needle injector of the first applicator 126, is close to, or at least partially overlapping, with the second area of the skin, which is defined mainly by a contact area of the heating unit 112 and the skin 102 of the subject. Particularly, the second area of the skin can be configured as a ring, wherein the first applicator 126 is located in the middle of the ring for associated administering.

The medical device 100 can be configured such that the skin-conditioning unit 110 of the medical device 100 can be configured to be detachable from the applicator unit 120, and the skin-conditioning unit 110 can be configured to operate independently from the applicator unit 120.

The slide bearing connection 606 of the medical device 100 can be configured to be detachable for a separation of the skin-conditioning unit 110 and the applicator unit 120. This configuration of the medical device 100 allows the skin-conditioning unit 110 of the medical device 100 to be spatially separated from the applicator unit 120 by mechanically and/or electrically decoupling the skin-conditioning unit 110 from the application unit 120, for administering conditioning measures to the skin 102 by use of the skin-conditioning unit 110, which is separated.

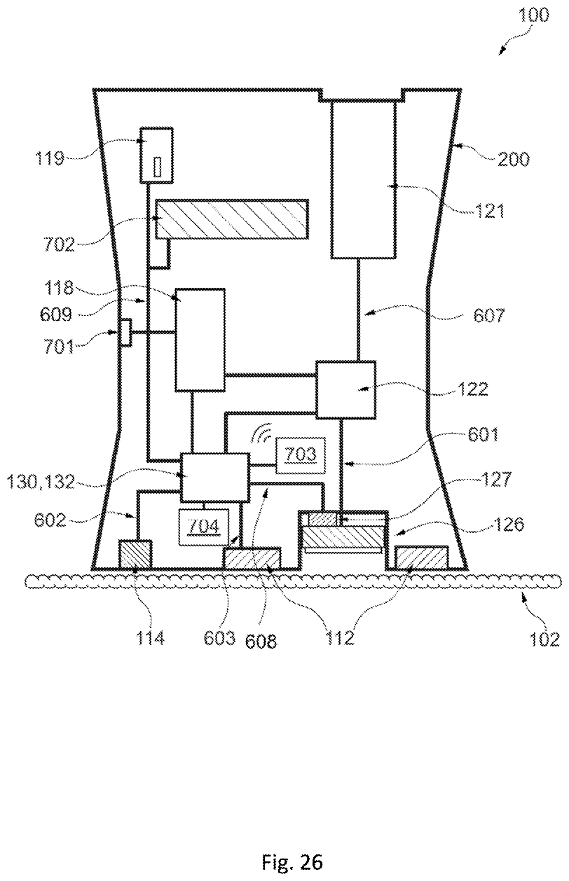

FIG. 26 sketches schematically a seventh embodiment of a medical device 100, wherein the medical device 100 includes a skin-conditioning unit, which is configured to apply conditioning measures to the skin 102, preferably for modifying a condition of the skin 102, and an applicator unit, whereby the elements of the skin-conditioning unit 110 and the applicator unit 120 are as mentioned above but spatially distributed within a housing 200 of the medical device 100.

The skin-conditioning unit and the applicator unit are configured by an interaction of operating of the heating unit 112, which is configured to provide heat to a second area of the skin 102, and operating of the first applicator 126, which is configured to administer a first therapeutic agent to a first area of the skin 102 to interact cooperatively, by means of the control unit 130, 132. Thus, the amount of the first therapeutic agent, which is stored within the first reservoir 121, is administered cooperatively by means of the first applicator 126 with an amount of conditioning measures by means of the heat source unit 112 of the skin-conditioning unit.

For this, the control unit 132 is coupled and can be controlled by a controller 130 of the medical device 100. The control unit 130, 132 is electrically coupled 603 to the heat source unit 112 and coupled 608 to the applicator actuator 127, which is mechanically coupled to the first applicator 126, and the control unit 132 is coupled 602 to a contact detection means 114, and also coupled to the pump 122, and coupled 609 to a trigger 119 and coupled to an user display 702. Additionally, the control unit 130, 132 is coupled to a storage area 704, and the control unit 130, 132 is coupled to a wireless coupling means 703.

For this embodiment, the dosage unit is realized by related stored data at the storage area 704 in respect to an configurable amount of the first therapeutic agent to be administered to the skin and/or to a configurable setting for a penetration depth of the skin 102 by the first applicator 126 and/or a configurable amount of heat provided by the heating unit 112.

The controller 130, 132 is configured to control the heating unit 112 and the first applicator to cooperatively interact to administer to the skin of the subject the amount of the first therapeutic agent by means of the pump 122 and the applicator actuator 127 with an amount of heat by means of the heating unit 112.

The first applicator 126 is configured to administer an amount of the first therapeutic agent with an amount of heat by means of the heating unit 112, wherein the first applicator 126 and respectively the pump 122 is controlled by the control unit 130, 132 for cooperative interaction.

The first therapeutic agent is provided by the first reservoir 121 by means of a pump 122, wherein the input of the pump 122 is coupled 607 with the first reservoir 121, and an output of the pump 122 is coupled 601 to the first applicator 126 for providing of the first therapeutic agent to the first applicator 126.

By means of an energy source 118 electrical power is provided to the pump 122, and also provided to the controller 130 including the control unit 132, and also provided to the heating unit 112, and provided to the applicator actuator 127, and provided to a contact detection means 114. The energy source 118 is configured with an electrical coupling to an external connector 701 to be recharged.

The contact detection means 114 is configured for monitoring of a proximity of the heating unit 112 with the surface of the skin 102, wherein the contact detection means 114 is coupled 602 to the controller 130, 132. By this, the controller 130 can control as well a cooperative interaction of the skin-conditioning unit, which is implemented here as the heating unit 112, and the first applicator 126, and the pump 122 to administer the first therapeutic agent to a skin 102 of a subject, as also to control a correct handling of the medical device 100 during the cooperative interaction.

Additionally, the medical device 100 includes a trigger 119, wherein the trigger 119 is configured to start the cooperative interaction of the heating unit 112 and the first applicator 126 and the pump 122 to administer to a skin of a subject an amount of the first therapeutic agent with an amount of conditioning measures, wherein the conditioning measures in this case is an administration of heat to the skin by means of the heating unit 112. Therefore, the trigger 119 is coupled to the controller 130, 132.

This embodiment of the medical device 100 also includes a wireless coupling means 116, which is coupled to the controller 130, 132, for enabling a signally coupling with an external device, to configure a specified depth of penetration of the skin 102 of the subject by the first applicator 126, which can include a needle injection unit, and/or to configure a specified amount for administering the first therapeutic agent to the skin 102 and/or to configure a specified amount of heat to be administered to the skin 102. That means that data exchanged and/or received by the wireless coupling device 703 can be stored within the storage area 704 for use of the controller 130, 132. The user display 702 is coupled to the controller 130, 132 to provide information about the stored data in the storage area 704 and/or for user instructions in respect to a correct use of the medical device 100. The access of the storage area 704 can be restricted to specific external devices and/or to specific users of the external devices.

The expressions “include” and “comprise” may be used interchangeably.

If not mentioned otherwise, it is to be understood that the expression “the medical devices” refers to any or all of the medical devices, particularly the medical device (100) and/or (X), more particularly the medical device (100), as mentioned in any of the embodiments described herein. Furthermore, if not mentioned otherwise, it is to be understood that any of the embodiments described herein in plural with reference to the medical devices refer independently to any of the medical devices, particularly the medical device (100) and/or (X), more particularly the medical device (100), as mentioned in any of the embodiments described herein.

It is further to be understood that any of the embodiments mentioned under the outline note ‘As further regards the medical device (100) for any of the embodiments as described herein’ is independently applicable and/or combinable with any of the embodiments of the medical devices, particularly as described under the outline note ‘As further regards independently any or all of the pharmaceutical compositions, the injectable dosage forms, the topical dosage forms, the medical devices and/or the kits of parts for any of the embodiments as described herein’.

In case of any irresolvable inconsistency or discrepancy in the embodiments of the medical device (100) and/or (X) as stated under this outline note ‘As further regards the medical device (100) for any of the embodiments as described herein’ and stated elsewhere in the description, but not under this outline note, it is to be understood that the embodiment under this outline note is preferred. The expressions “irresolvable inconsistency” or “irresolvable discrepancy” do not include alternative embodiments.

As further regards independently any or all of the kits of parts, particularly the kits of parts (I), (II), (III), (IV), (V) and/or (X), for any of the embodiments as described herein

Preferably, in the kit of parts the substance(s) and/or skin-conditioning agent(s) are provided in the form of any pharmaceutical acceptable formulation like for instance in the form of pharmaceutical compositions, topical dosage forms, medical devices and/or injectable dosage forms. Preferably, in the kit of parts the substance(s) and/or skin-conditioning agent(s) are provided in the form of any of the pharmaceutical compositions, the topical dosage forms, the injectable dosage forms, the medical devices or any combination thereof as mentioned in any of the embodiments described herein.

Preferably, the energizing means is any energizing means as mentioned in any of the embodiments described herein.

Preferably, the kits of parts comprise particles, particularly particles for drug delivery. The particles are preferably any of the particles as mentioned in any of the embodiments described herein.

Preferably, in the kit of parts the substance(s), where applicable, the energizing means is a heating means.

Preferably, the kit of parts comprise an instruction manual, handbook or medication leaflet.

If not mentioned otherwise, it is to be understood that any embodiment or definition described herein, particularly any embodiment or definition of the conditioning energy, the energizing means or the heating means described herein for step (A-6) is independently and mutatis mutandis applicable to the energizing means or the heating means, respectively, as mentioned in respect of the kits of parts.

A kit of parts (V) is proposed, which can include
an applicator unit; and
a skin-conditioning unit; and
optionally a reservoir, comprising a immunomodulatory substance.

Preferably, the reservoir is configured to be fluidly coupled to the applicator unit; and wherein the skin-conditioning and/or the applicator unit is configured to be mechanically and/or electrically coupled to cooperatively interact to administer to a skin of a subject an amount of a first therapeutic agent by means of the applicator unit with an amount of conditioning measures by means of the skin-conditioning unit.

Advantageously, the kit of parts enables a subject to use the medical device directly after the medical device is handed over to the subject, because the respective therapeutic agent can be stored in a different way than the rest of the medical device to optimize the storage conditions for the different components of the medical device. This can be particularly important because of deterioration of the respective therapeutic agent and/or because of vulnerable sterile conditions, for instance of injection needles and/or to take into account optimal conditions to store an energy source like for instance a battery or accumulator. For instance, the applicator unit, which may comprise an energy source like a battery may be recharged or may protected from coldness, the skin-conditioning unit comprising the therapeutic agent can be stored separately in a cool place.

Advantageously, the medical device is configured such that the skin-conditioning unit of the medical device can be configured to be detachable from the applicator unit as described above, to store the different units according to an optimal condition of the environment of the storage.

Preferably, the kit of parts include a further reservoir, comprising a further immunomodulatory substance.

Preferably, the kit of parts further include an applicator, wherein preferably the applicator comprises a syringe and/or an injection needle and/or a microneedle array.

If not mentioned otherwise, it is to be understood that any embodiment or definition of the applicator unit, the skin-conditioning unit, the reservoir and/or the immunomodulatory substance described herein in any of the embodiments of the medical device is independently and mutatis mutandis applicable to the applicator unit, the skin-conditioning unit, the reservoir and/or the immunomodulatory substance as mentioned for the kit of parts (V).

If not mentioned otherwise, it is to be understood that the expression “the kits of parts” refers to any or all of the kits of parts, particularly the kit of parts (I), (II), (III), (IV), (V) and/or (X), more particularly the kit of parts (I), (II), (III), (IV) and/or (V), as mentioned in any of the embodiments described herein. Furthermore, if not mentioned otherwise, it is to be understood that any of the embodiments described herein in plural with reference to the kits of parts refer independently to any of the kits of parts, particularly the kits of parts

(I), (II), (III), (IV), (V) and/or (X), more particularly the kits of parts (I), (II), (III), (IV) and/or (V), as mentioned in any of the embodiments described herein.

It is further to be understood that any of the embodiments mentioned under the outline note ‘As further regards independently any or all of the kits of parts, particularly the kits of parts (I), (II), (III), (IV), (V) and/or CO, for any of the embodiments as described herein’ is independently applicable and/or combinable with any of the embodiments of the medical devices, particularly as described under the outline note ‘As further regards independently any or all of the pharmaceutical compositions, the injectable dosage forms, the topical dosage forms, the medical devices and/or the kits of parts for any of the embodiments as described herein’.

Further general remarks, disclaimers, embodiments and aspects relating to any of the embodiments as mentioned herein and throughout the description are described in the following.

Preferably, the inflammatory disease, immunological disease and/or autoimmunological disease in any of the embodiments described herein is not caused by a genetic condition of the subject.

Preferably, the inflammatory disease, immunological disease and/or autoimmunological disease in any of the embodiments described herein is not caused by an antigen.

Preferably, the inflammatory disease, immunological disease and/or autoimmunological disease in any of the embodiments described herein is not caused by an allergen, e.g. bacteria.

Preferably, the immunomodulatory substance(s) as mentioned in any of the embodiments described herein does not comprise TNF-α (tumor necrosis factor alpha), TGF-β (transforming growth factor β), IL-6 (interleukin-6), IL-7 (interleukin-7) and/or IL-15 (interleukin-15).

Preferably, the immunomodulatory substance(s) as mentioned in any of the embodiments described herein is not an endogenous immunomodulatory substance(s) of the subject, a fragment or derivative thereof.

Preferably, the cytokine(s) and/or cytokine-like acting substance(s) as mentioned in any of the embodiments described herein is not an endogenous cytokine(s) and/or cytokine-like acting substance(s) of the subject, a fragment or derivative thereof. Preferably, the cytokine(s) and/or cytokine-like acting substance(s) does not comprise TGF-β, IL-6, IL-7 and/or IL-15.

Preferably, the interferon(s) and/or interferon-like acting substance(s) as mentioned in any of the embodiments described herein is not an endogenous interferon(s) and/or interferon-like acting substance(s) of the subject, a fragment or derivative thereof.

Preferably, the interleukin(s) and/or interleukin-like acting substance(s) as mentioned in any of the embodiments described herein is not an endogenous interleukin(s) and/or interleukin-like acting substance(s) of the subject, a fragment or derivative thereof. Preferably, the interleukin(s) and/or interleukin-like acting substance(s) does not comprise IL-6, IL-7 and/or IL-15.

Preferably, the neurotrophin(s) and/or neurotrophin-like acting substance(s) as mentioned in any of the embodiments described herein is not an endogenous neurotrophin(s) and/or neurotrophin-like acting substance(s) of the subject, a fragment or derivative thereof.

Preferably, the IFN-γ and/or IFN-γ-like acting substance(s) as mentioned in any of the embodiments described herein is not the subject's endogenous IFN-γ and/or IFN-γ-like acting substance(s), a fragment or derivative thereof. Preferably, any or all of the pharmaceutical compositions, injectable dosage forms, topical dosage forms and/or kits of parts as mentioned in any of the embodiments described herein does not comprise, preferably in effective amounts, and/or the method as mentioned in any of the embodiments described herein does not comprise administering, preferably in effective amounts, neither by topical application, neither by injection and/or neither by any other way, of the subject's endogenous IFN-γ and/or IFN-γ-like acting substance(s), a fragment or derivative thereof. More preferably, the subject's endogenous IFN-γ and/or IFN-γ-like acting substance(s), a fragment or derivative thereof, is not comprised or administered in an effective amount, even more preferably in an amount of 30,000 IU or less, still even more preferably is not comprised or administered in an amount in the range of zero to 30,000 IU. More preferably, the subject's endogenous IFN-γ and/or IFN-γ-like acting substance(s), a fragment or derivative thereof, is not comprised or administered in an in an effective amount, even more preferably in an amount of 1,500 ng or less, still even more preferably is not comprised or administered in an amount in the range of zero to 1,500 ng.

Preferably, the IFN-γ and/or IFN-γ-like acting substance(s) as mentioned in any of the embodiments described herein is not IFN-γ and/or IFN-γ-like acting substance(s), a fragment or derivative thereof, which is/are isolated from a vertebrate, preferably a mammal, more preferably a human or a mammal animal as defined above, more preferably the subject as defined in any of the embodiments according to the present invention.

Preferably, any or all of the pharmaceutical compositions, injectable dosage forms, topical dosage forms and/or kits of parts as mentioned in any of the embodiments described herein does not comprise, preferably in effective amounts, and/or the method as mentioned in any of the embodiments described herein does not comprise administering, preferably in effective amounts, neither by topical application, neither by injection and/or neither by any other way, of IFN-γ and/or IFN-γ-like acting substance(s), a fragment or derivative thereof, which is/are isolated from a vertebrate, preferably a mammal, more preferably a human or a mammal animal as defined above, more preferably the subject as defined in any of the embodiments according to the present invention. More preferably, such isolated IFN-γ and/or IFN-γ-like acting substance(s), a fragment or derivative thereof, is not comprised or administered in an effective amount, even more preferably in an amount of 30,000 IU or less, still even more preferably is not comprised or administered in an amount in the range of zero to 30,000 IU. More preferably, such isolated IFN-γ and/or IFN-γ-like acting substance(s), a fragment or derivative thereof, is not comprised or administered in an in an effective amount, even more preferably in an amount of 1,500 ng or less, still more preferably is not comprised or administered in an amount in the range of zero to 1,500 ng.

Preferably, the IL-2 and/or IL-2-like acting substance(s) as mentioned in any of the embodiments described herein is not the subject's endogenous IL-2 and/or IL-2-like acting substance(s), a fragment or derivative thereof.

Preferably, any or all of the pharmaceutical compositions, injectable dosage forms, topical dosage forms and/or kits of parts as mentioned in any of the embodiments described herein does not comprise, preferably in effective amounts, and/or the method as mentioned in any of the embodiments described herein does not comprise administering, preferably in effective amounts, neither by topical application, neither by injection and/or neither by any other way, of the subject's endogenous IL-2 and/or IL-2-like acting substance(s). More preferably, the subject's endogenous IL-2 and/or IL-2-like acting substance(s) is not comprised or administered in an effective amount, even more preferably in an amount of 30,000 IU or less, still even more preferably is not comprised or administered in an amount in the range of zero to 30,000 IU. More preferably, the subject's endogenous IL-2 and/or IL-2-like acting substance(s) is not comprised or administered in an effective amount, even more in an amount of 1,500 ng or less, still more preferably is not comprised or administered in an amount in the range of zero to 1,500 ng.

Preferably, the IL-2 and/or IL-2-like acting substance(s) in any of the embodiments described herein is not IL-2 and/or IL-2-like acting substance(s), a fragment or derivative thereof, which is/are isolated from a vertebrate, preferably a mammal, more preferably a human or a mammal animal as defined above, more preferably the subject as defined in any of the embodiments according to the present invention.

Preferably, any or all of the pharmaceutical compositions, injectable dosage forms, topical dosage forms and/or kits of parts as mentioned in any of the embodiments described herein does not comprise, preferably in effective amounts, and/or the method as mentioned in any of the embodiments described herein does not comprise administering, preferably in effective amounts, neither by topical application, neither by injection and/or neither by any other way, of IL-2 and/or IL-2-like acting substance(s), a fragment or derivative thereof, which is/are isolated from a vertebrate, preferably a mammal, more preferably a human or a mammal animal as defined above, more preferably the subject as defined in any of the embodiments according to the present invention. More preferably, such isolated IL-2 and/or IL-2-like acting substance(s) is not comprised or administered in an effective amount, even more preferably in an amount of 30,000 IU or less, still even more preferably is not comprised or administered in an amount in the range of zero to 30,000 IU. More preferably, such isolated IL-2 and/or IL-2-like acting substance(s) is not is not comprised or administered in an effective amount, even more in an amount of 1,500 ng or less, even more preferably is not comprised or administered in an amount in the range of zero to 1,500 ng.

Preferably, the IL-4 and/or IL-4-like acting substance(s) as mentioned in any of the embodiments described herein is not the subject's endogenous IL-4 and/or IL-4-like acting substance(s), a fragment or derivative thereof.

Preferably, any or all of the pharmaceutical compositions, injectable dosage forms, topical dosage forms and/or kits of parts as mentioned in any of the embodiments described herein does not comprise, preferably in effective amounts, and/or the method as mentioned in any of the embodiments described herein does not comprise administering, preferably in effective amounts, neither by topical application, neither by injection and/or neither by any other way, of the subject's endogenous IL-4 and/or IL-4-like acting substance(s). More preferably, the subject's endogenous IL-4 and/or IL-4-like acting substance(s) is not comprised or administered in an effective amount, even more preferably in an amount of 20,000 IU or less, still even more preferably is not comprised or administered in an amount in the range of zero to 20,000 IU. More preferably, the subject's endogenous IL-4 and/or IL-4-like acting substance(s) is not comprised or administered in an effective amount, even more in an amount of 1,000 ng or less, still more preferably is not comprised or administered in an amount in the range of zero to 1,000 ng.

Preferably, any or all of the pharmaceutical compositions, injectable dosage forms, topical dosage forms and/or kits of parts as mentioned in any of the embodiments described herein does not comprise, preferably in effective amounts, and/or the IL-4 and/or IL-4-like acting substance(s) as mentioned in any of the embodiments described herein is not IL-4 and/or IL-4-like acting substance(s), a fragment or derivative thereof, which is/are isolated from a vertebrate, preferably a mammal, more preferably a human or a mammal animal as defined above, more preferably the subject as defined in any of the embodiments according to the present invention.

Preferably, any or all of the pharmaceutical compositions, injectable dosage forms, topical dosage forms and/or kits of parts as mentioned in any of the embodiments described herein does not comprise, preferably in effective amounts, and/or the method as mentioned in any of the embodiments described herein does not comprise administering, preferably in effective amounts, neither by topical application, neither by injection and/or neither by any other way, of IL-4 and/or IL-4-like acting substance(s), a fragment or derivative thereof, which is/are isolated from a vertebrate, preferably a vertebrate, preferably a mammal, more preferably a human or a mammal animal as defined above, more preferably the subject as defined in any of the embodiments according to the present invention. More preferably, such isolated IL-4 and/or IL-4-like acting substance(s) is not comprised or administered in an effective amount, even more preferably in an amount of 20,000 IU or less, still even more preferably is not comprised or administered in an amount in the range of zero to 20,000 IU. More preferably, such isolated IL-4 and/or IL-4-like acting substance(s) is not comprised or administered in an effective amount, even more preferably in an amount of 1,000 ng or less, still even more preferably is not comprised or administered in an amount in the range of zero to 1,000 ng.

Preferably, the BDNF and/or BDNF-like acting substance(s) as mentioned in any of the embodiments described herein is not the subject's endogenous BDNF and/or BDNF-like acting substance(s), a fragment or derivative thereof. Preferably, any or all of the pharmaceutical compositions, injectable dosage forms, topical dosage forms and/or kits of parts as mentioned in any of the embodiments described herein does not comprise, preferably in effective amounts, and/or the method as mentioned in any of the embodiments described herein does not comprise administering, preferably in effective amounts, preferably in effective amounts, neither by topical application, neither by injection and/or neither by any other way, of the subject's endogenous BDNF and/or BDNF-like acting substance(s), a fragment or derivative thereof. More preferably, the subject's endogenous BDNF and/or BDNF-like acting substance(s), a fragment or derivative thereof, is not comprised or administered in an effective amount, even more preferably in an amount of 20,000 IU or less, still even more preferably is not comprised or administered in an amount in the range of zero to 20,000 IU. More preferably, the subject's endogenous BDNF and/or BDNF-like acting substance(s), a fragment or derivative thereof, is not comprised or administered in an effective amount, even more preferably in an amount of 500 ng or less, still even more preferably is not comprised or administered in an amount in the range of zero to 500 ng.

Preferably, the BDNF and/or BDNF-like acting substance(s) as mentioned in any of the embodiments described herein is not BDNF and/or BDNF-like acting substance(s), a fragment or derivative thereof, which is/are isolated from a vertebrate, preferably a mammal, more preferably a human or a mammal animal.

Preferably, any or all of the pharmaceutical compositions, injectable dosage forms, topical dosage forms and/or kits of parts as mentioned in any of the embodiments described herein does not comprise, preferably in effective amounts, and/or the method does not comprise administering, preferably in effective amounts, preferably in effective amounts, neither by topical application, neither by injection and/or neither by any other way, of BDNF and/or BDNF-like acting substance(s), a fragment or derivative thereof, which is/are isolated from a vertebrate, preferably a mammal, more preferably a human or a mammal animal. More preferably, such isolated BDNF and/or BDNF-like acting substance(s), fragment or derivative thereof, is not comprised or administered in an effective amount, even more preferably in an amount of 20,000 IU or less, still even more preferably is not comprised or administered in an amount in the range of zero to 20,000 IU, More preferably, such isolated BDNF and/or BDNF-like acting substance(s), fragment or derivative thereof, is not comprised or administered in an effective amount, even more preferably in an amount of 500 ng or less, still even more preferably is not comprised or administered in an amount in the range of zero to 500 ng.

Preferably, any or all of the pharmaceutical compositions, injectable dosage forms, topical dosage forms and/or kits of parts as mentioned in any of the embodiments described herein does not comprise, preferably in effective amounts, and/or the method as mentioned in any of the embodiments described herein does not comprise administering, preferably in effective amounts, preferably in effective amounts, neither by topical application, neither by injection and/or neither by any other way, cantharidin (2,6-Dimethyl-4,10-dioxatricyclo-[5.2.1.02,6]decane-3,5-dione), a fragment or derivative thereof. Preferably, the cantharidin, a fragment or derivative thereof is not comprised or administered in an effective amount, even more preferably in an amount of 10 mg or less, still even more preferably 1 mg or less, still more preferably is not comprised or administered in an amount in the range of zero to 10 mg, still even more preferably zero to 1 mg.

Preferably, any or all of the pharmaceutical compositions, injectable dosage forms, topical dosage forms and/or kits of parts as mentioned in any of the embodiments described herein does not comprise, preferably in effective amounts, and/or the method as mentioned in any of the embodiments described herein does not comprise administering, preferably in effective amounts, preferably in effective amounts, neither by topical application, neither by injection and/or neither by any other way, of glucocorticoid and/or glucocorticoid-protein, a fragment or derivative thereof. More preferably, glucocorticoid and/or glucocorticoid-protein, a fragment or derivative thereof, is not comprised or administered in an effective amount, even more preferably in an amount of 100 mg or less, still even more preferably is not comprised or administered in an amount in the range of zero to 100 mg.

Preferably, any or all of the pharmaceutical compositions, injectable dosage forms, topical dosage forms and/or kits of parts as mentioned in any of the embodiments described herein does not comprise, preferably in effective amounts, and/or the method as mentioned in any of the embodiments described herein does not comprise administering, preferably in effective amounts, preferably in effective amounts, neither by topical application, neither by injection and/or neither by any other way, of subject's endogenous glucocorticoid and/or glucocorticoid-protein, a fragment or derivative thereof. More preferably, the subject's endogenous glucocorticoid and/or glucocorticoid-protein, a fragment or derivative thereof is not comprised or administered in an effective amount, even more preferably in an amount of 100 mg or less, even more preferably is not comprised or administered in an amount in the range of zero to 100 mg.

Preferably, any or all of the pharmaceutical compositions, injectable dosage forms, topical dosage forms and/or kits of parts as mentioned in any of the embodiments described herein does not comprise, preferably in effective amounts, and/or the method as mentioned in any of the embodiments described herein does not comprise administering, preferably in effective amounts, preferably in effective amounts, neither by topical application, neither by injection and/or neither by any other way, of TNF-α and/or TNF-α-like acting substances, a fragment or derivative thereof. More preferably, TNF-α and/or TNF-α-like acting substances, a fragment or derivative thereof, is not comprised or administered in an effective amount, even more preferably in an amount of 30,000 IU or less, still even more preferably is not comprised or administered in an amount in the range of zero to 30,000 IU. More preferably, TNF-α and/or TNF-α-like acting substances, a fragment or derivative thereof, is not comprised or administered in an effective amount, even more preferably in an amount of 1,500 ng or less, still even more preferably is not comprised or administered in an amount in the range of zero to 1,500 ng.

Preferably, any or all of the pharmaceutical compositions, injectable dosage forms, topical dosage forms and/or kits of parts as mentioned in any of the embodiments described herein does not comprise, preferably in effective amounts, and/or the method as mentioned in any of the embodiments described herein does not comprise administering, preferably in effective amounts, preferably in effective amounts, neither by topical application, neither by injection and/or neither by any other way, of subject's endogenous TNF-α and/or TNF-α-like acting substances, a fragment or derivative thereof. More preferably, the subject's endogenous TNF-α and/or TNF-α-like acting substances, a fragment or derivative thereof is not comprised or administered in an effective amount, even more preferably in an amount of 30,000 IU or less, even more preferably is not comprised or administered in an amount in the range of zero to 30,000 IU. More preferably, the subject's endogenous TNF-α and/or TNF-α-like acting substances, a fragment or derivative thereof is not comprised or administered in an effective amount, even more preferably in an amount of 1,500 ng or less, still even more preferably is not comprised or administered in an amount in the range of zero to 1,500 ng.

Preferably, any or all of the pharmaceutical compositions, injectable dosage forms, topical dosage forms and/or kits of parts as mentioned in any of the embodiments described herein does not comprise, preferably in effective amounts, and/or the method as mentioned in any of the embodiments described herein does not comprise administering, preferably in effective amounts, neither by topical application, neither by injection and/or neither by any other way, of TNF-α and/or TNF-α-like acting substances, a fragment or derivative thereof, which is/are isolated from a vertebrate, preferably a mammal, more preferably a human or a mammal animal. More preferably, such isolated TNF-α and/or TNF-α-like acting substances, a fragment or derivative thereof is not comprised or administered in an effective amount, even more preferably in an amount of 30,000 IU or less, still even more preferably is not comprised or administered in an amount in the range of zero to 30,000 IU. More preferably, such isolated TNF-α and/or TNF-α-like acting substances, a fragment or derivative thereof is not comprised or administered in an effective amount, even more preferably in an amount of 1,500 ng or less, still more preferably is not comprised or administered in an amount in the range of zero to 1,500 ng.

Preferably, any or all of the pharmaceutical compositions, injectable dosage forms, topical dosage forms and/or kits of parts as mentioned in any of the embodiments described herein does not comprise, preferably in effective amounts, and/or the method as mentioned in any of the embodiments described herein does not comprise administering, preferably in effective amounts, preferably in effective amounts, neither by topical application, neither by injection and/or neither by any other way, of TGF-β and/or TGF-β-like acting substances, a fragment or derivative thereof. More preferably, TGF-β and/or TGF-β-like acting substances, a fragment or derivative thereof, is not comprised or administered in an effective amount, even more preferably in an amount of 30,000 IU or less, still even more preferably is not comprised or administered in an amount in the range of zero to 30,000 IU. More preferably, TGF-β and/or TGF-β-like acting substances, a fragment or derivative thereof, is not comprised or administered in an effective amount, even more preferably in an amount of 1,500 ng or less, still even more preferably is not comprised or administered in an amount in the range of zero to 1,500 ng.

Preferably, any or all of the pharmaceutical compositions, injectable dosage forms, topical dosage forms and/or kits of parts as mentioned in any of the embodiments described herein does not comprise, preferably in effective amounts, and/or the method as mentioned in any of the embodiments described herein does not comprise administering, preferably in effective amounts, preferably in effective amounts, neither by topical application, neither by injection and/or neither by any other way, of subject's endogenous TGF-β and/or TGF-β-like acting substances, a fragment or derivative thereof. More preferably, the subject's endogenous TGF-β and/or TGF-β-like acting substances, a fragment or derivative thereof is not comprised or administered in an effective amount, even more preferably in an amount of 30,000 IU or less, even more preferably is not comprised or administered in an amount in the range of zero to 30,000 IU. More preferably, the subject's endogenous TGF-β and/or TGF-β-like acting substances, a fragment or derivative thereof is not comprised or administered in an effective amount, even more preferably in an amount of 1,500 ng or less, still even more preferably is not comprised or administered in an amount in the range of zero to 1,500 ng.

Preferably, any or all of the pharmaceutical compositions, injectable dosage forms, topical dosage forms and/or kits of parts as mentioned in any of the embodiments described herein does not comprise, preferably in effective amounts, and/or the method as mentioned in any of the embodiments described herein does not comprise administering, preferably in effective amounts, neither by topical application, neither by injection and/or neither by any other way, of TGF-β and/or TGF-β-like acting substances, a fragment or derivative thereof, which is/are isolated from a vertebrate, preferably a mammal, more preferably a human or a mammal animal. More preferably, such isolated TGF-β and/or TGF-β-like acting substances, a fragment or derivative thereof is not comprised or administered in an effective amount, even more preferably in an amount of 30,000 IU or less, still even more preferably is not comprised or administered in an amount in the range of zero to 30,000 IU. More preferably, such isolated TGF-β and/or TGF-β-like acting substances, a fragment or derivative thereof is not comprised or administered in an effective amount, even more preferably in an amount of 1,500 ng or less, still more preferably is not comprised or administered in an amount in the range of zero to 1,500 ng.

Preferably, any or all of the pharmaceutical compositions, injectable dosage forms, topical dosage forms and/or kits of parts as mentioned in any of the embodiments described herein does not comprise, preferably in effective amounts, and/or the method as mentioned in any of the embodiments described herein does not comprise administering, preferably in effective amounts, preferably in effective amounts, neither by topical application, neither by injection and/or neither by any other way, of IL-6 and/or IL-6-like acting substance(s), a fragment or derivative thereof. More preferably, IL-6 and/or IL-6-like acting substance(s), a fragment or derivative thereof, is not comprised or administered in an effective amount, even more preferably in an amount of 30,000 IU or less, still even more preferably is not comprised or administered in an amount in the range of zero to 30,000 IU. More preferably, IL-6 and/or IL-6-like acting substance(s), a fragment or derivative thereof, is not comprised or administered in an effective amount, even more preferably in an amount of 1,500 ng or less, still even more preferably is not comprised or administered in an amount in the range of zero to 1,500 ng.

Preferably, any or all of the pharmaceutical compositions, injectable dosage forms, topical dosage forms and/or kits of parts as mentioned in any of the embodiments described herein does not comprise, preferably in effective amounts, and/or the method as mentioned in any of the embodiments described herein does not comprise administering, preferably in effective amounts, preferably in effective amounts, neither by topical application, neither by injection and/or neither by any other way, of subject's endogenous IL-6 and/or IL-6-like acting substance(s), a fragment or derivative thereof. More preferably, the subject's endogenous IL-6 and/or IL-6-like acting substance(s), a fragment or derivative thereof is not comprised or administered in an effective amount, even more preferably in an amount of 30,000 IU or less, even more preferably is not comprised or administered in an amount in the range of zero to 30,000 IU. More preferably, the subject's endogenous IL-6 and/or IL-6-like acting substance(s), a fragment or derivative thereof is not comprised or administered in an effective amount, even more preferably in an amount of 1,500 ng or less, still even more preferably is not comprised or administered in an amount in the range of zero to 1,500 ng.

Preferably, any or all of the pharmaceutical compositions, injectable dosage forms, topical dosage forms and/or kits of parts as mentioned in any of the embodiments described herein does not comprise, preferably in effective amounts, and/or the method as mentioned in any of the embodiments described herein does not comprise administering, preferably in effective amounts, neither by topical application, neither by injection and/or neither by any other way, of IL-6 and/or IL-6-like acting substance(s), a fragment or derivative thereof, which is/are isolated from a vertebrate, preferably a mammal, more preferably a human or a mammal animal. More preferably, such isolated IL-6 and/or IL-6-like acting substance(s), a fragment or derivative thereof is not comprised or administered in an effective amount, even more preferably in an amount of 30,000 IU or less, still even more preferably is not comprised or administered in an amount in the range of zero to 30,000 IU. More preferably, such isolated IL-6 and/or IL-6-like acting substance(s), a fragment or derivative thereof is not comprised or administered in an effective amount, even more preferably in an amount of 1,500 ng or less, still more preferably is not comprised or administered in an amount in the range of zero to 1,500 ng.

Preferably, any or all of the pharmaceutical compositions, injectable dosage forms, topical dosage forms and/or kits of parts as mentioned in any of the embodiments described herein does not comprise, preferably in effective amounts, and/or the method as mentioned in any of the embodiments described herein does not comprise administering, preferably in effective amounts, preferably in effective amounts, neither by topical application, neither by injection and/or neither by any other way, of IL-7 and/or IL-7-like acting substance(s), a fragment or derivative thereof. More preferably, IL-7 and/or IL-7-like acting substance(s), a fragment or derivative thereof, is not comprised or administered in an effective amount, even more preferably in an amount of 30,000 IU or less, still even more preferably is not comprised or administered in an amount in the range of zero to 30,000 IU. More preferably, IL-7 and/or IL-7-like acting substance(s), a fragment or derivative thereof, is not comprised or administered in an effective amount, even more preferably in an amount of 1,500 ng or less, still even more preferably is not comprised or administered in an amount in the range of zero to 1,500 ng.

Preferably, any or all of the pharmaceutical compositions, injectable dosage forms, topical dosage forms and/or kits of parts as mentioned in any of the embodiments described herein does not comprise, preferably in effective amounts, and/or the method as mentioned in any of the embodiments described herein does not comprise administering, preferably in effective amounts, preferably in effective amounts, neither by topical application, neither by injection and/or neither by any other way, of subject's endogenous IL-7 and/or IL-7-like acting substance(s), a fragment or derivative thereof. More preferably, the subject's endogenous IL-7 and/or IL-7-like acting substance(s), a fragment or derivative thereof is not comprised or administered in an effective amount, even more preferably in an amount of 30,000 IU or less, even more preferably is not comprised or administered in an amount in the range of zero to 30,000 IU. More preferably, the subject's endogenous IL-7 and/or IL-7-like acting substance(s), a fragment or derivative thereof is not comprised or administered in an effective amount, even more preferably in an amount of 1,500 ng or less, still even more preferably is not comprised or administered in an amount in the range of zero to 1,500 ng.

Preferably, any or all of the pharmaceutical compositions, injectable dosage forms, topical dosage forms and/or kits of parts as mentioned in any of the embodiments described herein does not comprise, preferably in effective amounts, and/or the method as mentioned in any of the embodiments described herein does not comprise administering, preferably in effective amounts, neither by topical application, neither by injection and/or neither by any other way, of IL-7 and/or IL-7-like acting substance(s), a fragment or derivative thereof, which is/are isolated from a vertebrate, preferably a mammal, more preferably a human or a mammal animal. More preferably, such isolated IL-7 and/or IL-7-like acting substance(s), a fragment or derivative thereof is not comprised or administered in an effective amount, even more preferably in an amount of 30,000 IU or less, still even more preferably is not comprised or administered in an amount in the range of zero to 30,000 IU. More preferably, such isolated IL-7 and/or IL-7-like acting substance(s), a fragment or derivative thereof is not comprised or administered in an effective amount, even more preferably in an amount of 1,500 ng or less, still more preferably is not comprised or administered in an amount in the range of zero to 1,500 ng.

Preferably, any or all of the pharmaceutical compositions, injectable dosage forms, topical dosage forms and/or kits of parts as mentioned in any of the embodiments described herein does not comprise, preferably in effective amounts, and/or the method as mentioned in any of the embodiments described herein does not comprise administering, preferably in effective amounts, preferably in effective amounts, neither by topical application, neither by injection and/or neither by any other way, of IL-15 and/or IL-15-like acting substance(s), a fragment or derivative thereof. More preferably, IL-15 and/or IL-15-like acting substance(s), a fragment or derivative thereof, is not comprised or administered in an effective amount, even more preferably in an amount of 30,000 IU or less, still even more preferably is not comprised or administered in an amount in the range of zero to 30,000 IU. More preferably, IL-15 and/or IL-15-like acting substance(s), a fragment or derivative thereof, is not comprised or administered in an effective amount, even more preferably in an amount of 1,500 ng or less, still even more preferably is not comprised or administered in an amount in the range of zero to 1,500 ng.

Preferably, any or all of the pharmaceutical compositions, injectable dosage forms, topical dosage forms and/or kits of parts as mentioned in any of the embodiments described herein does not comprise, preferably in effective amounts, and/or the method as mentioned in any of the embodiments described herein does not comprise administering, preferably in effective amounts, preferably in effective amounts, neither by topical application, neither by injection and/or neither by any other way, of subject's endogenous IL-15 and/or IL-15-like acting substance(s), a fragment or derivative thereof. More preferably, the subject's endogenous IL-15 and/or IL-15-like acting substance(s), a fragment or derivative thereof is not comprised or administered in an effective amount, even more preferably in an amount of 30,000 IU or less, even more preferably is not comprised or administered in an amount in the range of zero to 30,000 IU. More preferably, the subject's endogenous IL-15 and/or IL-15-like acting substance(s), a fragment or derivative thereof is not comprised or administered in an effective amount, even more preferably in an amount of 1,500 ng or less, still even more preferably is not comprised or administered in an amount in the range of zero to 1,500 ng.

Preferably, any or all of the pharmaceutical compositions, injectable dosage forms, topical dosage forms and/or kits of parts as mentioned in any of the embodiments described herein does not comprise, preferably in effective amounts, and/or the method as mentioned in any of the embodiments described herein does not comprise administering, preferably in effective amounts, neither by topical application, neither by injection and/or neither by any other way, of IL-15 and/or IL-15-like acting substance(s), a fragment or derivative thereof, which is/are isolated from a vertebrate, preferably a mammal, more preferably a human or a mammal animal. More preferably, such isolated IL-15 and/or IL-15-like acting substance(s), a fragment or derivative thereof is not comprised or administered in an effective amount, even more preferably in an amount of 30,000 IU or less, still even more preferably is not comprised or administered in an amount in the range of zero to 30,000 IU. More preferably, such isolated IL-15 and/or IL-15-like acting substance(s), a fragment or derivative thereof is not comprised or administered in an effective amount, even more preferably in an amount of 1,500 ng or less, still more preferably is not comprised or administered in an amount in the range of zero to 1,500 ng.

Preferably, any or all of the pharmaceutical compositions, injectable dosage forms, topical dosage forms and/or kits of parts as mentioned in any of the embodiments described herein does not comprise, preferably in effective amounts, and/or the method as mentioned in any of the embodiments described herein does not comprise administering, preferably in effective amounts, neither by topical application, neither by injection and/or neither by any other way, of insulin, a fragment or derivative thereof. More preferably, insulin, a fragment or derivative thereof is not comprised or administered in an effective amount, even more preferably in an amount of 500 IU or less, still even more preferably is not comprised or administered in an amount in the range of zero to 500 IU. More preferably, insulin, a fragment or derivative thereof is not comprised or administered in an effective amount, even more preferably in an amount of 1,500 ng or less, even more preferably is not comprised or administered in an amount in the range of zero to 1,500 ng.

Preferably, any or all of the pharmaceutical compositions, injectable dosage forms, topical dosage forms and/or kits of parts as mentioned in any of the embodiments described herein does not comprise, preferably in effective amounts, and/or the method as mentioned in any of the embodiments described herein does not comprise administering, preferably in effective amounts, neither by topical application, neither by injection and/or neither by any other way, of subject's endogenous insulin, a fragment or derivative thereof. More preferably, the subject's endogenous insulin, a fragment or derivative thereof is not comprised or administered in an effective amount, even more preferably in an amount of 500 IU or less, still even more preferably is not comprised or administered in an amount in the range of zero to 500 IU. More preferably, the subject's endogenous insulin, a fragment or derivative thereof is comprised or administered in an effective amount, even more in an amount of 1,500 ng or less, still more preferably is not comprised or administered in an amount in the range of zero to 1,500 ng.

Preferably, any or all of the pharmaceutical compositions, injectable dosage forms, topical dosage forms and/or kits of parts as mentioned in any of the embodiments described herein does not comprise, preferably in effective amounts, and/or the method as mentioned in any of the embodiments described herein does not comprise administering, preferably in effective amounts, neither by topical application, neither by injection and/or neither by any other way, of insulin, a fragment or derivative thereof which is/are isolated from a vertebrate, preferably a mammal, more preferably a human or a mammal animal. More preferably, such isolated insulin, a fragment or derivative thereof is not comprised or administered in an effective amount, even more preferably in an amount of 500 IU or less, still even more preferably is not comprised or administered in an amount in the range of zero to 500 IU. More preferably, such isolated insulin, a fragment or derivative thereof is not comprised or administered in an effective amount, even more in an amount of 1,500 ng or less, still more preferably is not comprised or administered in an amount in the range of zero to 1,500 ng.

Preferably, any or all of the pharmaceutical compositions, injectable dosage forms, topical dosage forms and/or kits of parts as mentioned in any of the embodiments described herein does not comprise, preferably in effective amounts, and/or the method as mentioned in any of the embodiments described herein does not comprise administering, preferably in effective amounts, neither by topical application, neither by injection and/or neither by any other way, of a body fluid like blood, serum, exudate, lymph fluid, wound fluid and/or blister fluid. More preferably, such body fluid is a lymph fluid, wound fluid and/or blister fluid extracted from a lymph fluid accumulation, would fluid accumulation and/or blister caused by cantharidin and/or cantharidin treatment, even more preferably blister fluid extracted from a blister caused by cantharidin. More preferably, such body fluid is not comprised or administered in an effective amount, even more preferably in an amount in the range of 50 ml or less, still even more preferably is not comprised or administered in an amount in the range of zero to 50 ml, even more preferably 10 ml or less, even more preferably is not comprised or administered in an amount in the range of zero to 10 ml, wherein preferably the amount refers to the undiluted body fluid prior to any potential dilution. The Expression “blood” in respect of body fluids comprises whole blood, untreated blood, blood treated with anticoagulation substances (e.g. EDTA, heparin), blood for intrinsic blood therapy, etc. The Expression “blood” in respect of body fluids does not comprise or refer to PBMCs as defined in the present invention.

Preferably, any or all of the pharmaceutical compositions, injectable dosage forms, topical dosage forms and/or kits of parts as mentioned in any of the embodiments described herein does not comprise, preferably in effective amounts, and/or the method as mentioned in any of the embodiments described herein does not comprise administering, preferably in effective amounts, neither by topical application, neither by injection and/or neither by any other way, of a subject's endogenous body fluid like blood, serum, exudate, lymph fluid, wound fluid and/or blister fluid. More preferably, such subject's endogenous body fluid is a lymph fluid, wound fluid and/or blister fluid extracted from a lymph fluid accumulation, would fluid accumulation and/or blister caused by cantharidin and/or cantharidin treatment, even more preferably blister fluid extracted from a blister caused by cantharidin. More preferably, such subject's endogenous body fluid is not comprised or administered in an effective amount, even more preferably in an amount in the range of 50 ml or less, still even more preferably is not comprised or administered in an amount in the range of zero to 50 ml, preferably of 10 ml or less, even more preferably is not comprised or administered in an amount in the range of zero to 50 ml, wherein more preferably the amount refers to the undiluted body fluid prior to any potential dilution.

Preferably, any or all of the pharmaceutical compositions, injectable dosage forms, topical dosage forms and/or kits of parts as mentioned in any of the embodiments described herein does not comprise, preferably in effective amounts, and/or the method as mentioned in any of the embodiments described herein does not comprise administering, preferably in effective amounts, preferably in step (B), step (B₁) and/or step (C), neither by topical application, neither by injection and/or neither by any other way, of a body fluid like blood, serum, exudate, lymph fluid, wound fluid and/or blister fluid, wherein such body fluid is which isolated from a vertebrate, preferably a mammal, more preferably a human or a mammal animal. More preferably, such isolated body fluid is a lymph fluid, wound fluid and/or blister fluid extracted from a lymph fluid accumulation, would fluid accumulation and/or blister caused by cantharidin and/or cantharidin treatment, even more preferably blister fluid extracted from a blister caused by cantharidin. More preferably, such isolated body fluid is not comprised or administered in an effective amount, even more preferably in an amount in the range of 50 ml or less, still even more preferably is not comprised or administered in an amount in the range of zero to 50 ml, preferably of 10 ml or less, even more preferably is not comprised or administered in an amount in the range of zero to 10 ml, wherein more preferably the amount refers to the undiluted body fluid prior to any potential dilution.

Preferably, any or all of the pharmaceutical compositions, injectable dosage forms, topical dosage forms and/or kits of parts as mentioned in any of the embodiments described herein does not comprise, preferably in effective amounts, and/or the method as mentioned in any of the embodiments described herein does not comprise administering, preferably in effective amounts, preferably in step (B), step (B₁) and/or step (C), neither by topical application, neither by injection and/or neither by any other way, of exudate, lymph fluid, wound fluid and/or blister fluid, like blister fluid extracted from a blister caused by cantharidin, in an effective amount, more preferably in an amount in the range of 50 ml or less, even more preferably 10 ml or less, still more preferably it is not comprised or administered in an amount in the range of zero to 10 ml, wherein more preferably the amount refers to the undiluted body exudate, lymph fluid, wound fluid and/or blister fluid, like blister fluid extracted from a blister caused by cantharidin prior to any potential dilution. Preferably, the exudate, lymph fluid, wound fluid and/or blister fluid like blister fluid extracted from a blister caused by cantharidin, is the subject's endogenous exudate, lymph fluid, wound fluid and/or blister fluid.

Preferably, any or all of the pharmaceutical compositions, injectable dosage forms, topical dosage forms and/or kits of parts as mentioned in any of the embodiments described herein does not comprise, preferably in effective amounts, and/or the method as mentioned in any of the embodiments described herein does not comprise administering, preferably in effective amounts, preferably in step (B), step (B₁) and/or step (C), neither by topical application, neither by injection and/or neither by any other way of any substance(s) as active ingredient(s) which is/are not similar or identical to any peptide, protein or any fragment of these naturally occurring in a vertebrate, preferably a mammal, more preferably a human or a mammal animal. More preferably, such substance(s) does not have an amino acid sequence identity to any peptide, protein or any fragment of these naturally occurring in a vertebrate, preferably a human or a mammal, more preferably in the subject as defined in any of the embodiments according to the present invention, of 85% or more, even more preferably 90% or more, still more preferably 95% or more, still even more preferably 97% or more, further preferably 98% or more, still even further preferably 99% or more and 100% or less. Preferably, the amino acid sequence identity includes or refers to substitutions, insertions and/or deletions of amino acid residues in respect to the naturally occurring peptide, protein or any fragment of these. More preferably, any or all of the pharmaceutical compositions, injectable dosage forms, topical dosage forms and/or kits of parts does not comprise, preferably in effective amounts, and/or the method does not comprise administering, preferably in effective amounts, preferably in step (B) and/or step (C), neither by topical application, neither by injection and/or neither by any other way of any substance(s) as active ingredient(s) other than a cytokine(s), still more preferably, other than immune-related cytokine(s). More preferably, such substance(s) is not comprised or administered as active ingredient(s) in an effective amount, even more preferably in an amount of 100 mg or less, even more preferably is not comprised or administered in an amount in the range of zero to 100 mg.

Preferably, any or all of the pharmaceutical compositions, injectable dosage forms, topical dosage forms and/or kits of parts as mentioned in any of the embodiments described herein does not comprise, preferably in effective amounts, and/or the method as mentioned in any of the embodiments described herein does not comprise administering, preferably in effective amounts, preferably in effective amounts, platelets. More preferably, platelets are not comprised or administered in an effective amount, even more preferably in an amount, preferably a total amount, of 3·10¹¹ platelets or less, even more preferably is not comprised or administered in an amount in the range of zero to 3·10¹¹ platelets.

Preferably, any or all of the pharmaceutical compositions, injectable dosage forms, topical dosage forms and/or kits of parts as mentioned in any of the embodiments described herein does not comprise, preferably in effective amounts, and/or the method as mentioned in any of the embodiments described herein does not comprise administering, preferably in effective amounts, preferably in step (B), step (B₁) and/or step (C), neither by topical application, neither by injection and/or neither by any other way of any antigen, autoantigen, allergen a and/or a fragment or derivative of these. More preferably, antigen, autoantigen, allergen and/or a fragment or derivative of these, is not comprised or administered in an amount sufficient to elicit a cytotoxic T-cell response. More preferably, antigen, autoantigen, allergen and/or a fragment or derivative of these, is not comprised or administered in an amount sufficient to elicit a cytotoxic T-cell response or less, even more preferably is not comprised or administered in an amount in the range of zero to any amount sufficient to elicit a cytotoxic T-cell response. Such antigen may be for instance tumour associated antigens, bacteria, viruses and/or a fragment or derivative of these.

Preferably, any or all of the pharmaceutical compositions, injectable dosage forms, topical dosage forms and/or kits of parts as mentioned in any of the embodiments described herein does not comprise, preferably in effective amounts, and/or the method as mentioned in any of the embodiments described herein does not comprise administering, preferably in effective amounts, preferably in step (B), step (B₁) and/or step (C), neither by topical application, neither by injection and/or neither by any other way of any antigen presenting cells. More preferably, antigen presenting cells are not comprised or administered in an amount sufficient to elicit a cytotoxic

T-cell response. More preferably, antigen presenting cells are not comprised or administered in an amount sufficient to elicit a cytotoxic T-cell response or less, even more preferably is not comprised or administered in an amount in the range of zero to any amount sufficient to elicit a cytotoxic T-cell response.

Preferably, any or all of the pharmaceutical compositions, injectable dosage forms, topical dosage forms and/or kits of parts as mentioned in any of the embodiments described herein does not comprise, preferably in effective amounts, and/or the method as mentioned in any of the embodiments described herein does not comprise administering, preferably in effective amounts, preferably in step (B), step (B₁) and/or step (C), neither by topical application, neither by injection and/or neither by any other way of any Toll-like receptor (TLR) and/or a fragment or derivative thereof. More preferably, TLR and/or a fragment or derivative thereof, is not comprised or administered in an amount sufficient to elicit an innate immunity response. More preferably, TLR and/or a fragment or derivative thereof is not comprised or administered in an amount sufficient to elicit an innate immunity response or less, even more preferably is not comprised or administered in an amount in the range of zero to any amount sufficient to elicit an innate immunity response.

Preferably, the administration in step (B), (B₁) and/or (C) in any of the embodiments described herein is not any or all selected from: oral, intravenous, intramuscular, intrathecal, sublingual, buccal, rectal, vaginal, ocular, nasal, anal and/or via the lungs, e.g. by inhalation.

Preferably, the method as mentioned in any of the embodiments described herein does not comprise a step requiring any or all selected from: an oral, intravenous, intramuscular, intrathecal, sublingual, buccal, rectal, vaginal, ocular, nasal, anal administration and/or an administration via the lungs, e.g. by inhalation.

Preferably, in any of the embodiments described herein, the method does not comprise any culturing and/or incubation of the PBMCs externally of the subject's body. Preferably, the PBMCs are blood PBMCs and/or isolated PBMCs, even more preferably isolated PBMCs. More preferably, the PBMCs are lymphocytes, more preferably naïve PBMCs, even more preferably naïve lymphocytes, still more preferably naïve B-cells and/or naïve T-cells, still even more preferably naïve T-cells.

More preferably, in any of the embodiments described herein, the method does not comprise any culturing and/or incubation of the PBMCs externally of the subject's body:
for an effective duration; and/or
for 1 hour or more, even more preferably 12 hour or more, still more preferably 1 day or more, still even more preferably 3 day or more, further preferably 7 days or more and 4 weeks or less, preferably 2 weeks or less; and/or
at 35° C. or more, preferably at 36° C. or more, even more preferably at 37° C. or more and preferably 50° C. of less; and/or using a cell incubator for an effective duration; and/or
under an atmosphere having an CO₂-content increased in comparison to air, preferably under a CO₂ atmosphere of 1% CO₂ or more, more preferably a CO₂ atmosphere of 5% CO₂ or more; and/or
in the presence of TGF-β (transforming growth factor β), for instance in an amount of about 50 pg/ml TGF-β or less (picograms/millilitre), of IL-6 (interleukin-6), of IL-7 (interleukin-7), of IL-15 (interleukin-15), of CD3 (cluster of differentiation 3) and/or of CD28 (cluster of differentiation 28) in an effective amount; and/or
with any cell culturing medium, like serum-free or serum-containing medium, in effective amounts.

In any of the embodiments described herein, the method does not does not exclude freezing of PBMCs.

Hence, in any of the embodiments described herein, the method may comprise a step of freezing the PBMCs prior to administration, preferably at −165° C. or more, more preferably in liquid nitrogen

In any of the embodiments described herein, the method does not does not exclude premixing of the PBMCs with one or more of the immunomodulatory substance(s) prior to administration, preferably 24 hours or less, more preferably 12 hours or less, even more preferably 6 hours or less, still more preferably 3 hours or less, still even more preferably 1.5 hours or less prior to administration.

Hence, in any of the embodiments described herein, the method may comprise a step of mixing the PBMCs with one or more of the immunomodulatory substance(s) prior to administration, preferably 24 hours or less, more preferably 12 hours or less, even more preferably 6 hours or less, still more preferably 3 hours or less, still even more preferably 1.5 hours or less prior to administration.

More preferably, particularly in case when in step (B) IL-4 and in step (B₁) BDNF is administered, the skin area [b] and the skin area [b₁] in any of the embodiments described herein do not overlap and/or they are distanced apart or the overlap is minimized. Even more preferably the skin area [b], overlapping skin area [a]/[b] and/or overlapping skin area [a]/[b]/[c] do not overlap and/or they are distanced apart to skin area [b₁], overlapping skin area [a]/[b₁] and/or overlapping skin areas [a]/[b₁]/[c] and/or the overlap thereof is minimized.

More preferably, particularly in case when in step (B) IL-4 and in step (B₁) BDNF is administered, the method as mentioned in any of the embodiments described herein is performed in such a way that no overlapping skin area [b]/[b₁] is formed or the overlapping skin area [b]/[b₁] is minimized.

More preferably, particularly in case when in step (B) IL-4 and in step (B₁) BDNF is administered, the skin area [b], overlapping skin area [a]/[b] and/or overlapping skin area [a]/[b]/[c], preferably overlapping skin area [a]/[b]/[c], of the first set of steps and the skin area [b₁], overlapping skin area [a]/[b₁] and/or overlapping skin area [a]/[b₁]/[c], preferably overlapping skin area [a]/[b₁]/[c], of the second set of steps in any of the embodiments described herein do not overlap and/or they are distanced apart or the overlap is minimized.

More preferably, particularly in case when in step (B) IL-4 and in step (B₁), BDNF is administered, the application area [b] and the application area [b₁] do not overlap and/or they are distanced apart or the overlap is minimized. Even more preferably the application area [b], overlapping application area [a]/[b] and/or overlapping application area [a]/[b]/[c] do not overlap and/or they are distanced apart to application area [b₁], overlapping application area [a]/[b₁] and/or overlapping application areas [a]/[b₁]/[c] and/or the overlap thereof in any of the embodiments described herein is minimized.

More preferably, particularly in case when in step (B) IL-4 and in step (B₁) BDNF is administered, the method is performed in such a way that no overlapping application area [b]/[b₁] is formed or the overlapping application area [b]/[b₁] is minimized.

More preferably, particularly in case when in step (B) IL-4 and in step (B₁) BDNF is administered, the application area [b], overlapping application area [a]/[b] and/or overlapping application area [a]/[b]/[c], preferably overlapping application area [a]/[b]/[c], of the first set of steps and the application area [b₁], overlapping application area [a]/[b₁] and/or overlapping application area [a]/[b₁]/[c], preferably overlapping application area [a]/[b₁]/[c], of the second set of steps in any of the embodiments described herein do not overlap and/or they are distanced apart or the overlap is minimized.

More preferably, particularly in case when in step (B) IL-4 and in step (B₁) BDNF is administered, the method as mentioned in any of the embodiments described herein is performed in such a way that none of the skin areas [b] and the skin areas [b₁] do overlap and/or they are distanced apart or the overlap is minimized. Preferably, the distance of such distanced apart skin areas and/or overlapping skin areas is mentioned in any of the embodiments described herein.

More preferably, particularly in case when in step (B) IL-4 and in step (B₁) BDNF is administered, the method as mentioned in any of the embodiments described herein is performed in such a way that none of the skin areas [b] and the skin areas [b₁] do overlap and/or they are distanced apart or the overlap is minimized. Preferably, the distance of such distanced apart skin areas and/or overlapping skin areas mentioned in any of the embodiments described herein.

More preferably, particularly in case when in step (B) IL-4 and in step (B₁) BDNF is administered, the method as mentioned in any of the embodiments described herein is performed in such a way that none of the skin areas [b] and the skin areas [b₁] do overlap and/or are distanced apart or the overlap is minimized. Preferably, the distance of such distanced apart skin areas and/or overlapping skin areas is mentioned in any of the embodiments described herein.

Preferably, the distance of such distanced apart skin areas and/or overlapping skin areas is mentioned in any of the embodiments described herein.

Preferably, the skin and/or skin layer(s) of the skin area, specifically the skin area [a], [b] and/or if applicable, [b₁] and/or [c], as mentioned in any of the embodiments described herein are immunological inactive and/or unchallenged, preferably at least partially, more preferably entirely immunological inactive and/or unchallenged and, preferably spatially distanced to any site of immunological activity and/or challenge. Most preferably, the skin and/or skin layer(s) of the skin area, specifically application area [a], [b], [b₁] and/or [c], as mentioned in any of the embodiments described herein are entirely immunological inactive and/or unchallenged and spatially distanced to any site of immunological activity and/or challenge as defined above.

Preferably, the skin and/or skin layer(s) of the application area, specifically application area [a], [b], [b₁] and/or [c], as mentioned in any of the embodiments described herein are immunological inactive and/or unchallenged, preferably at least partially, more preferably entirely immunological inactive and/or unchallenged and, preferably spatially distanced to any site of immunological activity and/or challenge.

Most preferably, the skin and/or skin layer(s) of the application area, specifically application area [a], [b], [b₁] and/or [c], as mentioned in any of the embodiments described herein are entirely immunological inactive and/or unchallenged and spatially distanced to any site of immunological activity and/or challenge as defined above.

Preferably, the skin-conditioning agent as mentioned in any of the embodiments described herein does not cause an allergic reaction in the subject.

Preferably, the pharmaceutical compositions, injectable dosage forms, topical dosage forms, medical devices and/or kits of parts as mentioned in any of the embodiments described herein is not and/or does not comprise any or all of an: oral, sublingual and/or buccal composition, e.g., a tablet, dragee, chewable tablet, drinkable solution, intravenous composition, intrathecal composition and/or intramuscular composition, ocular composition, nasal composition, anal composition, e.g. suppository, or a composition for inhalation into the lungs.

In any of the embodiments described herein, if not mentioned otherwise, the expression “dosage form suitable for injection” as mentioned in any of the embodiments described herein is in its meaning equivalent to the expression “injectable dosage form”. Hence, they have to be interpreted identically and may be used interchangeably.

In any of the embodiments described herein, if not mentioned otherwise, the expression “and/or” as mentioned in any of the embodiments described herein is in its meaning equivalent to the expression “selected from . . . , . . . , . . . , . . . or any combination thereof” and they are intended to cover all the permutations and combinations possible and equivalents that are not explicitly described. Hence, for instance, ‘w, x, y and/or z’ has to be interpreted identically and may be used interchangeably with ‘selected from w, x, y, z or any combination thereof’. Furthermore, if not mentioned otherwise, it is to be understood that in a list in which the last two member of the list are interconnected with “and/or” or “and” or “or”, the ‘and/or’ refers to all members of the list. For instance, ‘w, x, y and/or z’, ‘w, x, y and z’ and ‘w, x, y or z’ has to be interpreted identically and may be used interchangeably with ‘w and/or x and/or y and/or z’, w and x and y and z′ and ‘w or x or y or z’, respectively. Furthermore, if not mentioned otherwise, it is to be understood that embodiments or preferred embodiments stated with reference to a list containing two or more features interconnected with “and/or”, it is to be understood that the embodiments or preferred embodiments independently refer to each of the features or to any combination of two, more or all of the features stated in the list.

Moreover, in case any of the embodiments as described herein refers of a list of features interconnected with “and/or” it is to be understood that only the features applicable to the respective embodiment have to be considered, while the feature not covered by the embodiment are to be let out. For instance, if an embodiment relates to steps (A), (B), (B₁) and/or (C) of the method while the method, for instance as claimed, comprises only steps (A) and (B) and/or (C), it is to be understood that the embodiment has to be mutatis mutandis adapted to be understood as relating to steps (A), (B) and/or (C) only.

In any of the embodiments described herein, if not mentioned otherwise, the “s” stated in brackets, e.g. in any of the expressions like “substance(s)”, “immunomodulatory substance(s)”, “cytokine(s)”, “interferon(s)”, “interleukin(s)” and/or “neurotrophin(s)” etc., indicates in any of the embodiments described herein that it may be one or more, e.g. one or more substances, one or more immunomodulatory substances, one or more cytokines, one or more interferons, one or more interleukins, one or more neurotrophins, etc., respectively or e.g. a plurality of substances, a plurality of immunomodulatory substances, a plurality of cytokines, a plurality of interferons, a plurality of interleukins, a plurality of neurotrophins, etc., respectively. In case of more or a plurality of substances, immunomodulatory substances, cytokines, interferons, interleukins, neurotrophins, etc. such substances, immunomodulatory substances, cytokines, interferons, interleukins, neurotrophins, etc. may be different substances, immunomodulatory substances, cytokines, interferons, interleukins, neurotrophins, etc.

In any of the embodiments described herein, the “s” stated in brackets in any of the expressions like “substance(s)”, “immunomodulatory substance(s)”, “cytokine(s)”, “interferon(s)”, “interleukin(s)” and/or “neurotrophin(s)” etc. in any of the embodiments described herein is equivalent to “one or more immunomodulatory substances”, “one or more substances”, “one or more immunomodulatory substances”, “one or more cytokines”, “one or more interferons”, “one or more interleukins” and/or “one or more neurotrophins”, etc., respectively. In case of more substances, immunomodulatory substances, cytokines, interferons, interleukins, neurotrophins, etc. such substances, immunomodulatory substances, cytokines, interferons, interleukins, neurotrophins, etc. are typically different from each other. For instance, it is stated that cytokine(s) are administered. This may be interchangeably read as one or more cytokines are administered, wherein in case of more cytokines, the cytokines are different from each other, e.g. they may be IFN-γ and IL-2 or BDNF, IL-4 and IL-2. In case of more immunomodulatory substances such immunomodulatory substances are typically different from each other. In other words, in case of more cytokines, a “plurality of cytokines” may be administered, wherein the cytokines of the plurality of cytokines are different from each other.

Furthermore, it is to be understood that the expression “immunomodulatory substance(s)” in any of the embodiments described herein is equivalent to “one or more immunomodulatory substances”. In case of more immunomodulatory substances such immunomodulatory substances are typically different from each other. In other words, in case of more immunomodulatory substances, a “plurality of immunomodulatory substance” may be administered in the method as mentioned in any of the embodiments described herein, wherein the immunomodulatory substances of the plurality of immunomodulatory substances are different from each other.

In any of the embodiments described herein, if not mentioned otherwise, the expressions “temperature on the skin”, “temperature of the skin” and/or “skin surface temperature” may be used interchangeably.

In any of the embodiments described herein, if not mentioned otherwise, the expressions “redness on the skin”, “redness of the skin”, “skin redness” and/or “skin surface redness” may be used interchangeably.

In any of the embodiments described herein, if not mentioned otherwise, the expression “heat crème” or “heat cream” as mentioned in any of the embodiments described herein is in its meaning equivalent to the expression “heat crème”. Hence, they have to be interpreted identically and may be used interchangeably.

In any of the embodiments described herein, if not mentioned otherwise, the expression “optional” or “optionally” as mentioned in any of the embodiments described herein means that the subsequently described event, step or circumstance may or may not occur, and that the description includes instances where such event, step or circumstance occurs and instances in which it does not

In any of the embodiments described herein and/or throughout the specification, if not mentioned otherwise, when referred to a “the method”, the “method for treating and/or preventing” or the “method as mentioned in any of the embodiments described herein”, the “method for treating and/or preventing an inflammatory disease, immunological disease and/or autoimmunological disease of a subject” according to the present invention as described herein and/or as described herein is meant.

In any of the embodiments described herein, if not mentioned otherwise, “xxx or more” as mentioned in any of the embodiments described herein is in its meaning equivalent to the expression “xxx or more than xxx”. Hence, they have to be interpreted identically and may be used interchangeably. For instance, ‘1% or more’ is equivalent in its meaning to ‘1% or more than 1%’. Similarly, the expression “xxx or less” as mentioned in any of the embodiments described herein is in its meaning equivalent to the expression “xxx or less than xxx”. Therefore, they have to be interpreted identically and may be used interchangeably.

If not mentioned otherwise, the expression “in any of the embodiments described herein” or “as mentioned in any of the embodiments described herein” refers, where applicable, to any embodiment of the medical use, the method, the pharmaceutical compositions, the injectable dosage forms, the topical dosage forms, the medical devices and/or the kits of parts described herein.

In any of the embodiments described herein, if not mentioned otherwise, the sequence of method steps is presented in such a way that the process can be easily understood. The skilled person will recognize that the process steps can also be carried out in a different order to achieve to the same or a corresponding result. If not mentioned otherwise, in this sense, the order of the method steps can be changed accordingly. Hence, if not mentioned otherwise, the designation of steps with (A), (B), (B₁), (C), (A-1), (A-2), (A-3), (A-4), (A-5), (A-6), (A-7) and (A-8) do not imply any order.

Furthermore, in the embodiments of the present invention described herein some features are accompanied by number words, e.g. first immunomodulatory substance(s), second immunomodulatory substance(s), first activating unit, second activating unit, first reservoir etc., in order to improve readability or to make the assignment clearer, but, if not mentioned otherwise, this does not imply the existence of certain features in any of the embodiments described herein.

In any of the embodiments described herein, if not mentioned otherwise, the expression “comprising” does not exclude other elements or steps.

In any of the embodiments described herein, if not mentioned otherwise, the indefinite article “a” or “an” does not exclude a plurality.

The mere fact that certain measures are re-cited in mutually different dependent claims does not indicate that a combination of these measures cannot be used to advantage.

Materials and Methods

1. O2C—Oxygen to See

Measurement of the Blood Volume and Blood Flow within the Skin

(A) O2C—Method and Equipment

The blood volume and the blood flow within the skin capillaries have been measured using the technology O2C (“Oxygen to see” of LEA Medizintechnik GmbH, Giessen, Germany http://www.lea.de/deu/fro2chd.htm).

The O2C examination combines tissue photospectrometry (ATS) with laser Doppler flow measurement (LDA; Laser-Doppler-Anemometry). Laser Doppler Anemometry (LDA) is an optical measurement method for the punctual determination of velocity components in flows. A split laser beam crosses at the measuring point, where interference fringe patterns are created. A particle moving through the fringe pattern generates a scattered light signal whose frequency is proportional to the velocity component perpendicular to the interference fringes and is detected by the light detector. By combining laser Doppler systems with different laser wavelengths, all flow velocity components can be detected. In addition to capillary venous oxygen saturation and hemoglobin quantity, the flow velocity in the capillaries can be determined simultaneously.

Most of the capillary blood volume of the skin is pooled in the postcapillary venous system. Therefore, the O2C focusses on measuring in this area. With respect to the blood flow direction, the capillaries of the skin are arranged upstream from the postcapillary venous system. Hence, by measuring changes in the postcapillary venous system direct conclusions for changes in the capillaries of the skin can be drawn. An increase in the blood volume pooled in the postcapillary venous system measured by O2C results from an increase in the blood volume leaving the capillaries and arriving at the postcapillary venous system (for further explanations see below). Such increased blood volume arriving at the postcapillary venous system may be due to a vasodilation of the skin capillaries thereby allowing to hold a larger volume of blood or due to an increased throughput through the skin capillaries due to an accelerated blood flow or combination of both. Hence, by measuring the blood volume within the postcapillary venous system and the blood flow within the skin tissue, the O2C allows to determine the blood volume and the blood flow present within skin capillaries.

By combining Laser-Doppler flowmetry and tissue spectrometry for measurement, the O2C particularly measures the tissue micro-perfusion based on the following parameters:
oxygen saturation of hemoglobin (sO₂) in %;
relative hemoglobin amount (rHb) in arbitrary units (AU);
relative blood flow (rFlow) in arbitrary units (AU); and
blood flow velocity (Vel) in arbitrary units (AU).

A Laser-Doppler shift is caused by the movement of the erythrocytes within the lumen of the capillaries, which is then detected by the O2C-device as blood flow velocity (Vel).

The amount of erythrocytes is indirectly detected by the absorption of the hemoglobin. Emitted white light registers the hemoglobin oxygen saturation (sO₂) and the relative hemoglobin amount (rHb). The sO₂ is determined by the colour of the blood, the rHb is indicated by the absorption of the white light in the tissue.

The amount of erythrocytes correlates with the blood volume present. The blood volume combined with the blood flow velocity (Vel), yields the overall blood flow (rFlow).

For the O2C measurement white light of 500-630 nm wave length as well as laser light of 830 nm wave length have been used.

The O2C-device used for the measurements in the present invention had the manufacturer's serial number SN: 306-069-18 and was used in accordance with the manufacturer's manual “02C Handbuch fur Softwarevariante V40”, O2C-Handbuch-12-01-de-01-20191206, LEA Medizintechnik GmbH, www.LEA.de, if not mentioned otherwise. For this O2C-device different types of probes are available. Depending on the emitted light wave length as well as the distance between the illumination and the detection point (called separation), different tissue depths are evaluated. For the measurements four probes of the type LFx81 were used having a main working depth of 2 mm. The O2C measurements were performed at Friedrich Alexander University Erlangen-Nuernberg, University Hospital Erlangen, Department of Vascular Surgery on Dec. 22, 2021.

(B) O2C—Setup and Conditions for the Measurement

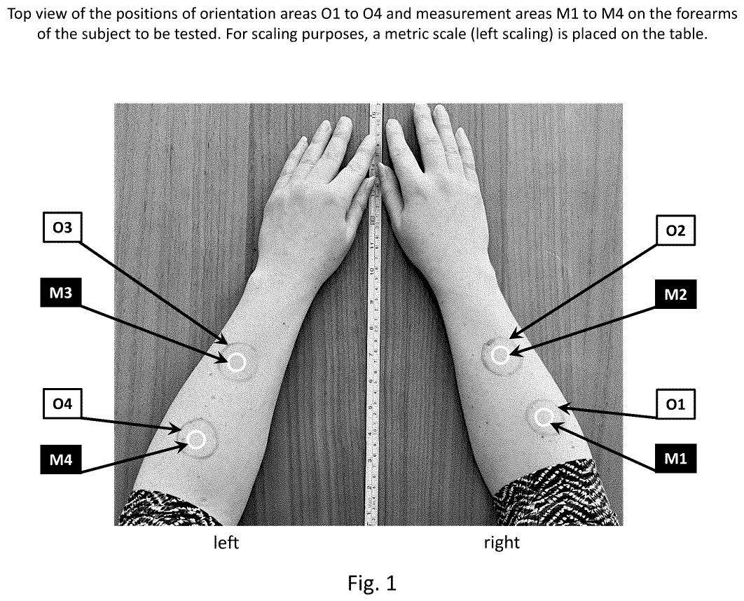

The skin of a subject on which the measurement was performed was cleaned and washed with hand soap approximately 5 hours before the measurement was started. No substances like crème, lotion oil etc. were applied to the skin after washing. The subject was placed on a chair with the forearms resting in a calm position on a table in front. Blood pressure and pulse were monitored and stayed constant without changes. The measurement was performed on both forearms of the subject. The skin of the two forearms was cleaned with an alcohol pad. On each forearm, two circles with approximately 3 cm in diameter were outlined with an eye liner pen to visually highlight the respective orientation areas, O1, O2, O3 and O4 (FIG. 1). For the measurements, four of the above-described probes of type LFx81 were used. Within each orientation area O1 to O4 one probe was placed and fixed in order to provide data of a respective measurement area M1, M2, M3 and M4. The measurement area is smaller than and located within the corresponding orientation area (FIG. 1). Since blood flow is easily influenced by pressure, the probes were applied to the tissue in such a way that no pressure was exerted or venous return was influenced. The probes were attached to the skin surface with the transparent double-sided adhesive foil strips supplied by LEA Medizintechnik.

The subject was instructed not to move or to tense the muscles in the arms for the entire durations of the measurement, which was also supervised.

The measurement of hemoglobin levels and oxygen saturation requires shielding from light. It was made sure that there were no strongly modulated light sources (e.g. irradiation with neon tubes) in the direct vicinity of the probe head, since the input of modulated extraneous light causes false readings in blood flow velocity and blood flow. The room was completely darkened by blinds. The only light sources present were the monitor of the O2C-device and a 24 inch computer screen. These lighting conditions were maintained and kept constant for all measurements. The distance between M1 and M2 as well as between M3 and M4 was approximately 7 cm measured centre to centre of the circles. The probes were fixed on the skin in such a way that they were not on the colour markings outlined with the eye liner pen.

The room temperature was kept constant at approx. 20° C. with no air flow, doors and windows remained closed. The conditions were not changed for all measurements.

After a resting time of 15 min to achieve acclimatisation to the environment and thereby ensure stable conditions, a baseline measurement was conducted to get starting values for the sO₂, rHb, rFlow and Vel.

Directly after the baseline measurement, the measurement using the heat pad was conducted as described in the Materials and Methods' section ‘O2C—Oxygen to See’, item (C).

(C) O2C-Measurement on a Subject when Using a Heat Pad

An O2C-measurement was performed on a subject following topical application of a heat to the skin. The equipment for the measurement and the measurement setup were used as described in this section under items (A) and (B) above.

A heat pad (“Relags Magic Heat Pad” by basic NATURE; size: 10 cm×10 cm; weight; 126 g; BSN350974) was activated by pressing and clicking the integrated metal disc activator. After solidification (crystallization) and opacification of the gel-like content of the heat pad was completed, the activated and warmed heat pad was immediately applied with slight pressure topically to measurement area O4. O4 was totally covered by the warmed heat pad for a duration of 1 min. The application was performed in accordance with the Materials and Methods' section ‘Application of a heat pad’.

The measurement areas M1 to M4 were treated or left untreated as follows:
M1=measurement area 1=proximal right forearm=untreated control;
   corresponding orientation area was O1
M2=measurement area 2=distal right forearm=untreated control;
   corresponding orientation area was O2
M3=measurement area 3=distal left forearm=untreated control;
   corresponding orientation area was O3
M4=measurement area 4=proximal left forearm=topical heat pad application;
   corresponding orientation area was O4

After removing the heat pad from O4, the four probes were placed and fixed on M1 to M4 as described above.

One minute after removing the heat pad, the determination of the values for the sO₂, rHb, rFlow and Vel was started and continued until the baseline levels had re-established.

(D) O2C-Measurement on a Subject when Using a Heat Crème

An O2C-measurement was performed on a subject following topical application of a heat crème. The equipment for the measurement and the measurement setup were used as described in this section under items (A) and (B) above.

A heat crème, Kytta® heat balm containing methylnicotinat [“Kytta® heat balm” by P&G Health Germany GmbH, Germany, PZN 12358936]) was used as a test composition. A crème basis lacking any active substances (Basiscrème DAC, Bombastus Werke AG, PZN 04193119; density 9.5 g/10 ml) was used as a comparative composition.

The measurement areas M1 to M4 were treated or left untreated as follows:
M1=measurement area 1=proximal right forearm=heat crème, Kytta® heat balm, test composition;
   corresponding orientation area was O1
M2=measurement area 2=distal right forearm=untreated control;
   corresponding orientation area was O2
M3=measurement area 3=distal left forearm=untreated control;
   corresponding orientation area was O3
M4=measurement area 4=proximal left forearm=crème basis, Basiscrème DAC, comparative composition;
   corresponding orientation area was O4

A separate eyeshadow applicator comprising a tip made of a flattened sponge (about 8 mm×10 mm×3 mm) was used to apply the test composition to M1 and the comparative composition to M4, respectively.

It was started with the application of the comparative composition. The applicator to be used for the Basiscrème DAC was weighed without heat crème, then 107 mg crème basis were applied to one side of the sponge and the crème basis was evenly distributed to totally cover the outlined area of M4. Then, with the other side of the applicator's sponge the excessive crème basis was removed from M4. Subsequently, the applicator was weighed again and it was calculated that 39 mg of Basiscrème DAC remained on M4. Hence, finally 39 mg Basiscrème DAC were applied to M4.

The time needed for the application of the comparative composition was about 15 sec to 20 sec.

Sixty seconds after the start of the application of the Basiscrème DAC to O4 it was started to apply the Kytta® heat balm to O1. For this purpose, another applicator to be used for the heat crème was weighed without heat crème, then 109 mg heat crème were applied to one side of the sponge and the heat crème was evenly distributed to totally cover the outlined area of O1. Then, with the other side of the applicator's sponge the excessive heat crème was removed from M1. Subsequently, the applicator was weighed again and it was calculated that 40 mg of Kytta® heat balm remained on O1. Hence, finally 40 mg Kytta® heat balm were applied to O1. The application was performed in accordance with the Materials and Methods' section ‘Application of heat crème’.

The time needed for the application of the test composition was also about 15 sec to 20 sec.

After finishing the application of the Kytta® heat balm to O1, all 4 areas were cleaned with an alcohol pad to ensure sticking of the probes, then the 4 probes were placed and fixed on M1 to M4 as described above.

Two minutes after finishing the application of the Kytta® heat balm, the determination of the values for the sO₂, rHb, rFlow and Vel was started and continued until baseline levels of sO₂, rFlow and Vel had re-established.

2. Thermal Measurement and Skin Reddening

(A) Method and equipment for thermal measurement of the skin surface temperature and quantification of skin reddening

The thermal measurement of the temperature on the skin, also referred to as skin surface temperature, and the quantification of skin reddening were performed simultaneously. The expressions “temperature on the skin”, “temperature of the skin” and/or “skin surface temperature” are used interchangeably.

Thermal Measurement

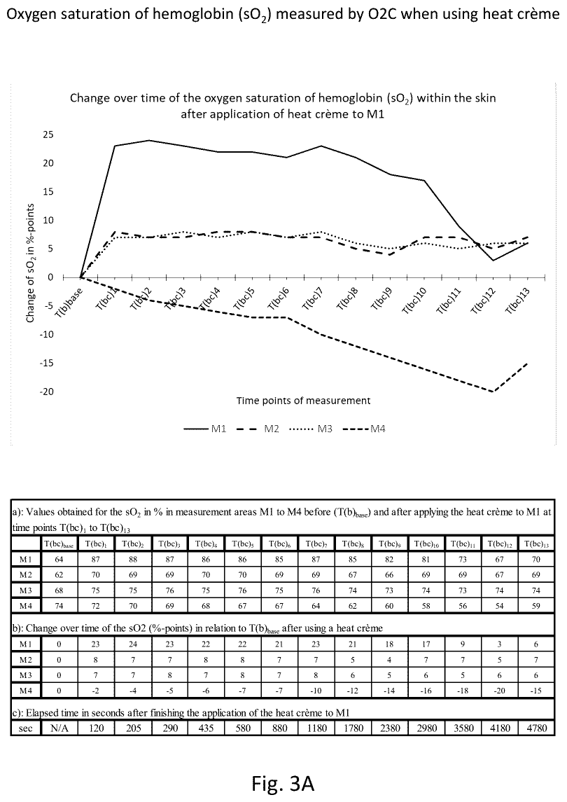

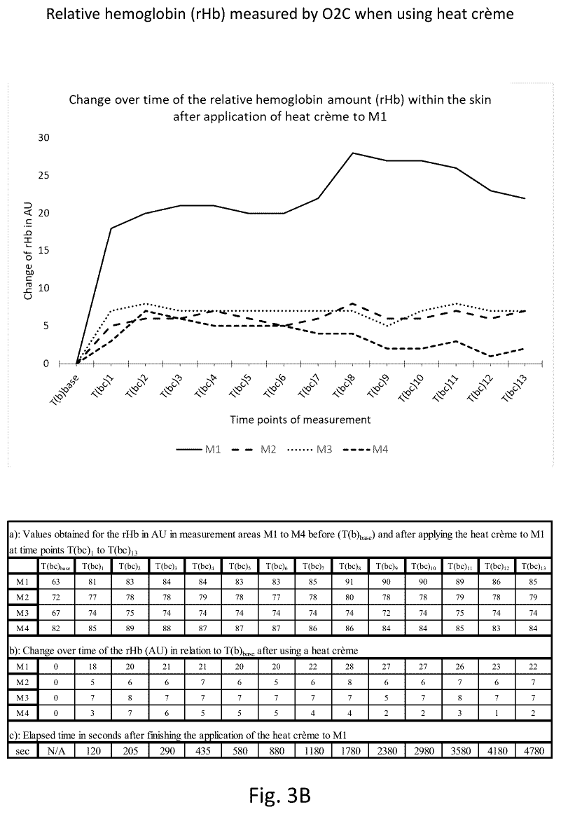

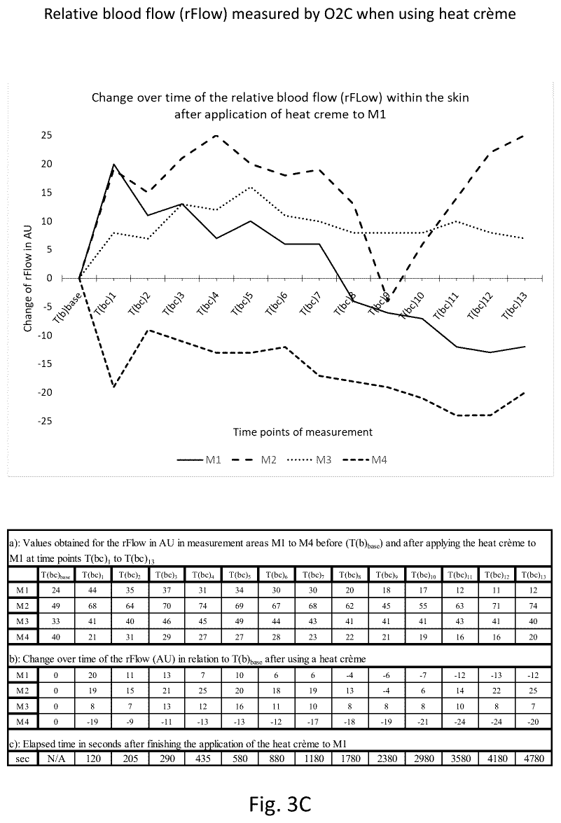

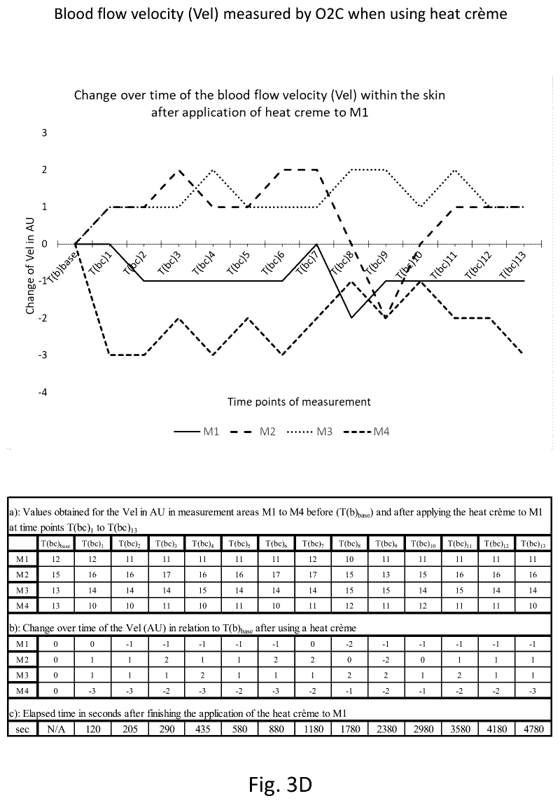

The thermal measurement of the temperature on the skin, also referred to as skin surface temperature, and the skin reddening, also referred to as skin surface temperature, was conducted with a thermographic camera (i.e. a thermographic infrared camera) firmly mounted on a tripod facing down on a table. The measurement setup of the thermographic camera is indicated and depicted in FIG. 3. A calibrated and cooled highly accurate thermographic camera, type InfraTec ImageIRO 8800, serial number 88173803, with a 25 mm F2.0 multicoated germanium lens, Type Janos Strix (serial 404790167) was used. The corresponding image aperture angle of the thermographic camera with this lens was 21.7°×17.5°. The sensor (photodetector) of the camera was a Stirling cooled photo quantum detector with 640×512 image pixels; 15 μm pixel pitch. The detector material was mercury cadmium telluride (MCT). The system spectral range was Long Wave Infrared (LWIR), from 7.7 μm to 10.2 μm. The applied readout method was ITR (Integrate Then Read). The system was set for the measurement to the calibrated measuring range of −10° C. to 40° C. where the system had a pre-specified absolute measurement precision of ±1° C. or ±1% and a specified relative resolution of 0.025 K (Kelvin) at 30° C. (degree Celsius). It turned out that the measured values exceeded the pre-set measurement range. Hence, the system was, while already set to −10° C. to 40° C., additionally qualified in the range of 40° C. to 45° C. and reached a maximum deviation of ±1° C. or ±1%. This was equal to the range of −10° C. to 40° C., resulting in the overall calibrated range of −10° C. to 45° C.

The acquisition frame rate was set to 1 f/s. The integration time (exposure time) was 347 its. The data bus to the recording host PC was gigabit ethernet.

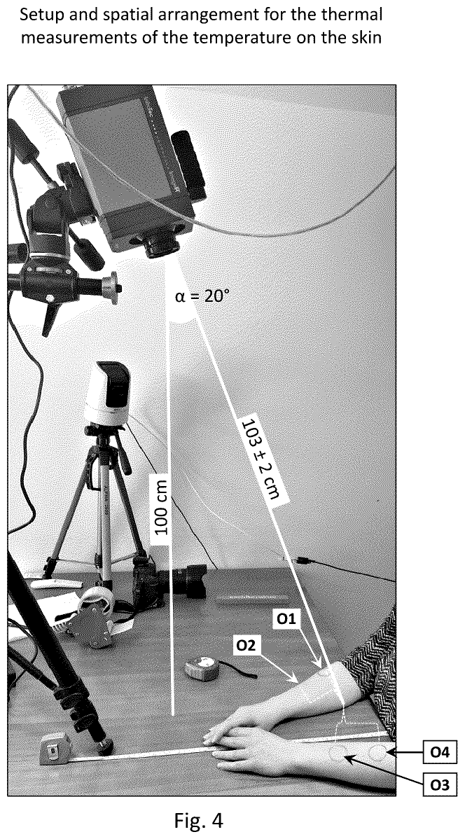

The thermographic camera acquired the temperature in image pixels. Each measurement area M1 to M4 was represented by a number of measurement pixels within the respective orientation areas O1 to O4. The arrangement of the measurement and orientation areas is shown in FIG. 1 wherein the orientation areas O1 to O4 are indicated by the dark line of an eye liner pen on the subject's forearms (FIGS. 1 and 4). Measurements areas M1 to M4 are in indicated in the figures by white encircled areas (FIGS. 1, 5, 7 and 9). All measurement areas M1 to M4 are a smaller subset of their respective orientation area O1 to O4 as can be seen in FIG. 1. All measurement pixels for each measurement area were recorded over time with one frame of 640×512 measured pixels, per second, which included the obtained measurement values (maximum and minimum temperatures data are not shown) and the average temperature per pixel. The average temperature of all measurement pixels within each individual measurement area M1 to M4 was taken as the temperature on the skin, also referred to as skin surface temperature or temperature of the skin.

For the thermal measurements, the surface emissivity (ε) of the measured object is relevant. Human skin is with a factor of 0.98 very close to the theoretical maximum of 1.0 (see e.g. Manual of Technical Temperature Measurement, 2^{nd} edition, VDI, Springer Verlag, chapter 13.7, p. 1235). Taking the correction according to the emissivity (ε) into account, for example, at the begin of the main measurement T(tc)ₚᵣₑ, the measured values for the measurement area M1=30.12° C. and M2=29.44° C. resulted in a delta of 0.68° C. The corrected value adopting the emissivity (ε) into the calculation would result in M1=30.26° C. and M2=29.57° C. (the real temperatures). The delta was here 0.69° C. The correction was in the second digit after the comma (0.68° C. versus 0.69° C.=0.01° C.) and therefore not relevant for the measurement described. For easier processing, the following values are rounded to one digit after the comma, representing all measured values without the correction factor of the emissivity (ε).

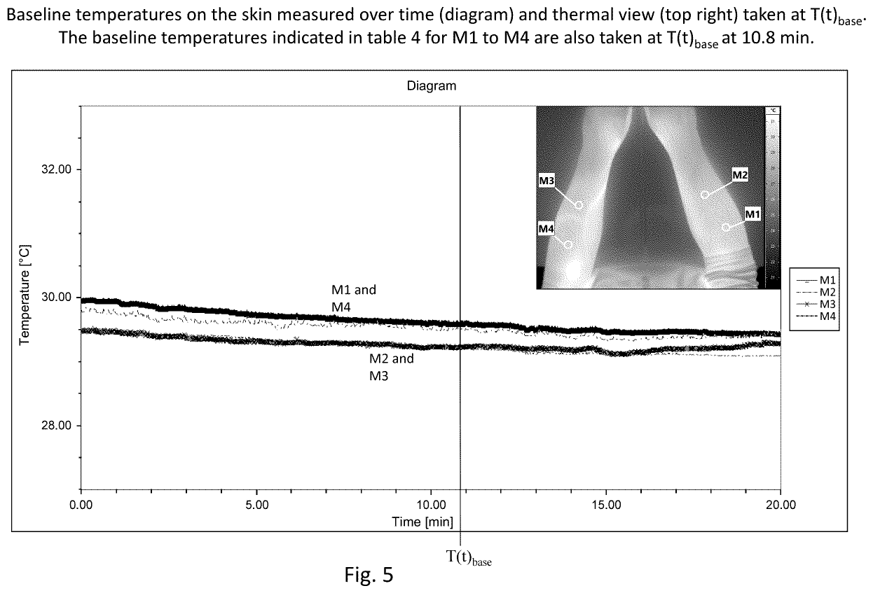

For the diagrams in FIGS. 5, 6 and 8, the average temperature (skin surface temperature) of all measurement pixels within the four measurement areas M1, M2, M3 and M4 was plotted over time.

Before the described measurement was started, a baseline measurement was done, to ensure stable operation during the measurement. Before the start of the baseline measurement, the system was activated in measuring mode with a pre-run time of 1264 sec (21.06 min).

The measurements were documented with a continuous recording of a standard Network Video camera Type Axis V5915 with Firmware 8.45.3.2 and recorded on an Axis Camera Station Video Server version 5.38.317 for reference.

(B) Setup and Conditions for Thermal Measurement

The skin of a subject on which the measurement was performed was cleaned and washed with hand soap approximately 3 hours before the measurement was started. No substances like crème, lotion oil etc. were applied to the skin after washing. The subject was placed on a chair with the forearms resting in a calm position on a table in front. The exact position was outlined on the table to assure that the forearms were not moved or flexed during the entire measurement process. On each forearm, two circles with approximately 3 cm in diameter were outlined with an eye liner pen to visually highlight the respective orientation areas, O1 to O4 (FIG. 1). The orientation areas were used to have a visually findable orientation on which substances were applied to the skin and a heat cushion was centered during its application. The measurement areas M1 to M4 were smaller in diameter and allocated within their respective orientation areas. The smaller measurement area allows small movements of the arm without effecting the measurements, since the measurement areas M1 to M4 are still within the larger orientation areas O1 to O4 where the treatment was applied (FIG. 1).

The thermographic camera was positioned above a table facing downwards with approx. 20° inclination towards the test person, focusing on the forearms (FIG. 4). The distance to each measurement area M1 to M4 on the forearms was 103±2 cm along the 20° inclined axis of the camera, wherein the distance from the lens to the table surface along the 90° vertical axis was 100 cm.

The room temperature at the beginning of the measurements was 22.0° C. The room temperature was passively kept constant during the entire measurements and did solely slightly rise in a slow and constant slope over a timeframe of approximately 6 hours by 0.5° C. to 22.5° C. at the end of the measurements. During the entire measurements, no air chill was applicable neither to the camera nor to the test person, no active air circulation (e.g. fan) was present, doors and windows remained closed and neither a heating system nor any cooling (air conditioning) was active.

To ensure stable measurement conditions, the skin surface temperature in all four measurement areas M1 to M4 was recorded for 20 min to serve as a baseline. About 20 min after finishing the determination of the baseline, the measurement using the heat pad was conducted as described in the Materials and Methods' section ‘Thermal measurement (skin surface temperature) and skin reddening on a subject when using a heat pad’, item (C). Furthermore, a measurement using a heat crème was conducted as described in the Materials and Methods' section ‘Thermal measurement (skin surface temperature) and skin reddening on a subject when using a heat crème’, item (D), about 2 hours after finishing the determination of the baseline.

(C) Thermal Measurement (Skin Surface Temperature) and Skin Reddening on a Subject when Using a Heat Pad

A measurement of the skin surface temperature was performed on a subject following topical application of a heat pad to the skin wherein the heat pad was centered on orientation area O4. The equipment for the measurement and the measurement setup were used as described in this section under above items (A) and (B).

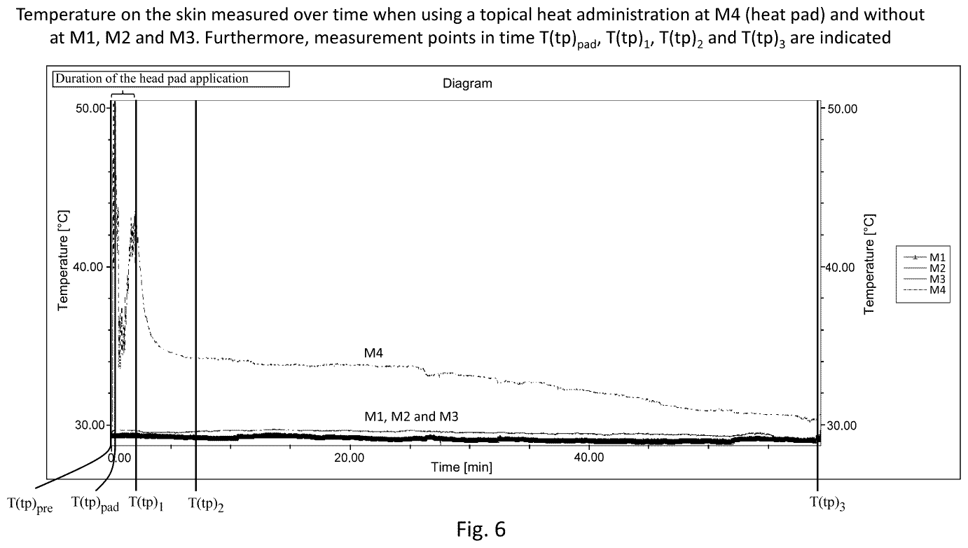

Before applying the heat pad to the skin, the measurement of the skin surface temperature was started and the temperature readings in measurement areas M1 to M4 were in a range of 29.2° C. to 29.6° C. (FIG. 6, Table 6).

Then, a heat pad (“Relags Magic Heat Pad” by basic NATURE; size: 10 cm×10 cm; weight; 126 g; BSN350974) was activated by pressing and clicking the integrated metal disc activator. After solidification (crystallization) and opacification of the gel-like content of the heat pad was completed, the activated and warmed heat pad was immediately applied with slight pressure topically to the skin of orientation area O4. O4 and respectively the included measurement area M4 was totally covered by the warmed heat pad for a duration of approximately 2 min in order to heat up the skin. The application was performed in accordance with the Materials and Methods' section ‘Application of a heat pad’. The warmed heat pad had a measured maximum temperature of 49.4° C. (T(tp)_{pad}) at its surface (FIG. 6, Table 6). A certain variation of the temperature in the diagram reading of FIG. 6 over the duration of the heat pad application was observed due to measurement uncertainty of the heat pad surface, as the emissivity (ε) of the heat pad surface could not be determined and might influence the temperature readings, and because the heat pad was partially covered by the hand of an operator person keeping the heat pad centered on O4.

The measurement areas M1 to M4 were treated or left untreated as follows:
M1=measurement area 1=proximal right forearm=untreated control;
   corresponding orientation area was 01
M2=measurement area 2=distal right forearm=untreated control;
   corresponding orientation area was 02
M3=measurement area 3=distal left forearm=untreated control;
   corresponding orientation area was 03
M4=measurement area 4=proximal left forearm=topical heat pad application;
   corresponding orientation area was 04

The temperature was continuously measured every second throughout the experiment. Beginning ten seconds after the heat pad was removed from M4 (at T(tp)₁), it can be seen from FIG. 6 that it took about 57 min (at T(tp)₃) until the measured temperature difference between the untreated controls M1 to M3 and the heat pad treated M4 was about 1.3° C. (approximately 1.4° C. above the average of the baseline skin surface temperature, Table 5).

The skin reddening was visually determined by observing and investigating orientation areas O1 to O4 with the naked eye.

(D) Thermal Measurement (Skin Surface Temperature) and Skin Reddening on a Subject when Using Heat Crème

A measurement of the skin surface temperature was performed on a subject following topical application of a heat crème (test composition) to the skin. The equipment for the measurement and the measurement setup were used as described in this section under items (A) and (B) above.

A heat crème, Kytta® heat balm containing methylnicotinat [“Kytta® heat balm” by P&G Health Germany GmbH, Germany, PZN 12358936]) was used as a test composition. A crème basis lacking any active substances (Basiscrème DAC, Bombastus Werke AG, PZN 04193119; density 9.5 g/10 ml) was used as a comparative composition. The measurement areas M1 to M4 were treated or left untreated as follows:
M1=measurement area 1=proximal right forearm=heat crème, Kytta® heat balm, test composition;
   corresponding orientation area was O1
M2=measurement area 2=distal right forearm=untreated control;
   corresponding orientation area was O2
M3=measurement area 3=distal left forearm=crème basis, Basscréme DAC, comparative composition;
   corresponding orientation area was O3
M4=measurement area 4=proximal left forearm=untreated control;
   corresponding orientation area was 04

Before applying the compositions to the skin, the measurement of the skin surface temperatures of M1 to M4 were started and continued for 110 minutes, until the temperature between the measurement areas M3 (comparative composition) on the left forearm and M1 (test composition) on the right forearm nearly leveled and reached a difference of about 1° C.

A separate eyeshadow applicator comprising a tip made of a flattened sponge (about 8 mm×10 mm×3 mm) was used to apply the test composition to O1 and the comparative composition to O3, respectively.

Then, it was started with the application of the comparative composition. The applicator to be used for the Basiscrème DAC was weighed without heat crème, then 99 mg crème basis were applied to one side of the sponge and the crème basis was evenly distributed to totally cover orientation area O3. Then, with the other side of the applicator's sponge the excessive crème basis was removed from O3. Subsequently, the applicator was weighed again and calculated that 51 mg of Basiscrème DAC remained on O3. Hence, finally 51 mg Basiscrème DAC were applied to O3.

The time needed for the application of the comparative composition was about 15 sec to 20 sec.

About sixty seconds after the start of the application of the Basiscrème DAC to O4 it was started to apply the Kytta® heat balm to orientation area O1. For this purpose, another applicator to be used for the heat crème was weighed without heat crème, then 92 mg heat crème were applied to one side of the sponge and the heat crème was evenly distributed to totally cover the outlined area of O1. Then, with the other side of the applicator's sponge the excessive heat crème was removed from O1. Subsequently, the applicator was weighed again and calculated that 65 mg of Kytta® heat balm remained on O1. Hence, finally 65 mg Kytta® heat balm were applied to O1. The application was performed in accordance with the Materials and Methods' section ‘Application of heat crème’.

The time needed for the application of the test composition was also about 15 sec to 30 sec.

The skin reddening was visually determined by observing and investigating orientation areas O1 to O4 with the naked eye.

3. Measurement of Areas and Distances Relating to the Skin

The areas and distances relating to the skin like the skin area, application area, effective surface area as well as spacings and distances on the skin were measured by placing a metric measuring tape on the surface of the skin and visually reading the extents like spacings, radii or distances. Areas are then calculated from the measured extents. Area sums are obtained by adding up the individually calculated areas. For easier handling, areas or distances to be measured may by be highlighted or enricles on the skin by using a skin compatible pen prior to measuring.

4. Preparation of PBMCs

All items and materials to be placed on the working place were extensively disinfected with 80% ethanol (v/v in aqua ad injectabilia, Braun) prior to placing them on the working place. Then, 30 μl sodium heparin (Heparin-Natrium supplied by Braun, B. Braun Melsungen AG, 25000 I.E./5 ml (25,000 IU/5 ml) Injection solution, PZN:15782698) were transferred to two 10 ml syringes each. One heparin equipped syringe was equipped with a 21G butterfly injection needle and used to collect venous blood from a patient. Then the filled syringe was exchanged with the second heparin equipped syringe and another 10 ml blood were collected (the needle remained in the vein of the patient so that he would not have to be pricked twice). The filled syringes were directly inverted three times in order to mix heparin and blood. The heparin blood was transferred to 10 ml sterile centrifugation tube. Four additional 10 ml sterile centrifugation tubes were equipped with 3 ml lymphocytes separation medium (Lymphocyte Separating Medium, Pancoll human, density: 1.077 g/ml, product number P04-60125). The lymphocytes separation medium was carefully overlaid with 5 ml of the heparin blood. These four additional 10 ml sterile centrifugation tubes were centrifuged for 15 min at 850×g without break in order to allow separation of mononuclear leucocytes from the blood by density gradient centrifugation.

The white interface containing PBMCs was carefully harvested by pipetting and transferred in fresh 10 ml centrifugation tubes. These tubes were centrifuged for 12 min at 1900×g. The supernatant was discarded and the cellular pellet was solved in 10 ml sterile 0.9% NaCl solution (0.9% Braun Ecoflac Plus, B. Braun Melsungen AG, PZN: 08609255) and centrifuged for 8 min at 750×g. The supernatant was discarded, the cellular pellet resuspended in 10 ml sterile 0.9% NaCl solution (0.9% Braun Ecoflac Plus, B. Braun Melsungen AG, PZN: 08609255) and centrifuged for 8 min at 750×g. The supernatant was discarded and the cellular pellet was resuspended in 0.25 ml 0.9% NaCl solution (0.9% Braun Ecoflac Plus, B. Braun Melsungen AG, PZN: 08609255). The PBMCs were counted using Trypan-Blue staining in combination with a Neubauer-hamocytometera as known in the art. The PBMCs' concentration was adjusted to 4.5×10⁶ PBMCs (4.5 million PBMCs) in 100 μl.

5. Preparation of Injection Solution Comprising IFN-γ: IFN-γ Injection Solution

High Dose IFN-γ Injection Solution: 10,000 IU/Ml IFN-γ:

From one ampule containing in total 2×10⁶ IU human IFN-γ (2,000,000 IU; 0.1 mg) in 0.5 ml injection solution (injection solution comprising recombinant human IFN-γ-1b-protein comprising the peptide sequence SEQ ID NO: 1, produced by Boehringer Ingelheim RCV GmbH & Co KG and in Germany commercially available from Boehringer Ingelheim Pharma GmbH & Co. KG under the tradename Imukin®, PZN: 06958744, Lot: M000487), 0.5 ml (containing 2,000,000 IU; 0.1 mg IFN-γ) were mixed with 199.5 ml 0.9% NaCl solution (Braun Ecoflac Plus, B. Braun Melsungen AG, PZN: 08609255). This resulted in a final concentration of 10,000 IU/ml and 500 ng/ml IFN-γ, respectively. The solution was frozen in 0.5 ml aliquots in sterile glass ampules and stored at −20° C. until use.

Prior to administration, the thus obtained high dose IFN-γ injection solution of one glass ampule was thawed at room temperature and 0.1 ml (100 μl) were then injected into the skin of the patient corresponding to an amount of 1,000 IU and 50 ng IFN-γ, respectively.

Low Dose IFN-γ Injection Solution: 1,000 IU/Ml IFN-γ:

If a lower concentration of IFN-γ was needed, a dilution was prepared as follows. One 500 μl aliquot of the above described high dose IFN-γ solution containing 10,000 IU/ml and 500 ng/ml IFN-γ, respectively, was thawed and mixed with 4.5 ml 0.9% NaCl solution (Kochsalzlösung 0.9% Miniplasco connect, B. Braun Melsungen AG, PZN: 03079870). From this dilution, 0.1 ml were injected into the skin of the patient corresponding to an amount of 100 IU and 5 ng IFN-γ, respectively.

Alternatively, from one ampule containing in total 2×10⁶ IU human IFN-γ (2,000,000 IU; 0.1 mg) in 0.5 ml injection solution (injection solution comprising recombinant human IFN-γ-1b-protein comprising the peptide sequence SEQ ID NO: 1, produced by Boehringer Ingelheim RCV GmbH & Co KG and in Germany commercially available from Boehringer Ingelheim Pharma GmbH & Co. KG under the tradename Imukin®, PZN: 06958744, Lot: M000487), 0.5 ml (containing 200,000 IU; 10 μg IFN-γ) were mixed with 199.5 ml 0.9% NaCl solution (Braun Ecoflac Plus, B. Braun Melsungen AG, PZN: 08609255). This resulted in a final concentration of 1,000 IU/ml and 50 ng/ml IFN-γ, respectively. The solution was frozen in 0.5 ml aliquots in sterile glass ampules and stored at −20° C. until use.

Prior to administration, the thus obtained low dose IFN-γ injection solution of one glass ampule was thawed at room temperature and 0.1 ml (100 μl) were then injected into the skin of the patient corresponding to an amount of 100 IU and 5 ng IFN-γ, respectively.

6. Preparation of Injection Solution Comprising IL-2: IL-2 Injection Solution

To one ampule containing 18×10⁶ IU (18,000,000 IU; 1.1 mg) lyophilized IL-2 (recombinant human IL-2-protein comprising the peptide sequence SEQ ID NO: 2 produced by Novartis Pharmaceuticals UK, Limited, and in Germany commercially available from Novartis Pharma GmbH under the tradename Proleukin®S) 9 ml water for injection (AQUA AD Injectabilia Miniplasco connect, B. Braun Melsungen AG, PZN 03113087) were added on order to solve the IL-2 to achieve a final concentration of 2×10⁶ IU/ml IL-2 (2,000,000 IU/ml; 122 μg/ml IL-2). Of this IL-2 solution, 0.4 ml were mixed with 399.6 ml 0.9% NaCl solution (Braun Ecoflac Plus PZN: 08609255). This resulted in a final concentration of 2,000 IU/ml and 122 ng/ml IL-2. The solution was frozen in 0.5 ml aliquots in sterile glass ampules.

Prior to administration, the thus obtained IL-2 injection solution of one glass ampule was thawed at room temperature and 0.1 ml (100 μl) were then injected into the skin of the patient, corresponding to an amount of 200 IU and 12.2 ng IL-2, respectively.

7. Preparation of Injection Solution Comprising IFN-γ and IL-2: IFN-γ/IL-2 Injection Solution

High Dose IFN-γ/IL-2 Injection Solution: 10,000 IU/Ml IFN-γ and 2,000 IU/Ml IL-2:

Two ampules containing each 2×10⁶ IU human IFN-γ (2,000,000 IU; 0.1 mg) in 0.5 ml injection solution (injection solution comprising recombinant human IFN-γ-1b-protein comprising the peptide sequence SEQ ID NO: 1, produced by Boehringer Ingelheim RCV GmbH & Co KG and in Germany commercially available from Boehringer Ingelheim Pharma GmbH & Co. KG under the tradename Imukin®, PZN: 06958744, Lot: M000487) were combined and the resulting 1 ml IFN-γ injection solution (4,000,000 IU/ml; 200 μg/ml IFN-γ) was mixed with 398.6 ml 0.9% NaCl solution (Braun Ecoflac Plus, B. Braun Melsungen AG, PZN: 08609255) resulting in 399.6 ml solution containing IFN-γ.

Then, to one ampule containing 18×10⁶ IU (18,000,000 IU; 1.1 mg) lyophilized IL-2 (recombinant human IL-2-protein comprising the peptide sequence SEQ ID NO: 4, produced by Novartis Pharmaceuticals UK, Limited, and in Germany commercially available from Novartis Pharma GmbH under the tradename Proleukin®S) 9 ml water for injection (AQUA AD Injectabilia Miniplasco connect, B. Braun Melsungen AG, PZN 03113087) were added in order to solve the IL-2 to achieve a final concentration of 2×10⁶ IU/ml IL-2 (2,000,000 IU/ml; 122 μg/ml IL-2). Of this IL-2 solution, 0.4 ml were mixed with the above prepared 399.6 ml solution containing IFN-γ. This resulted in a final concentration of 10,000 IU/ml IFN-γ and 2,000 IU/ml IL-2, and 500 ng IFN-γ and 122 ng IL-2, respectively.

The solution was frozen in 0.5 ml aliquots in sterile glass ampules and stored at −20° C. until use.

Prior to administration, the thus obtained high dose IFN-γ/IL-2 injection solution of one glass ampule was thawed at room temperature and 0.1 ml (100 μl) were then injected into the skin of the patient corresponding to an amount of 1,000 IU IFN-γ and 200 IU IL-2, and 50 ng IFN-γ and 12.2 ng IL-2, respectively.

Low Dose IFN-γ/IL-2 Injection Solution: 1,000 IU/Ml IFN-γ and 2,000 IU/Ml IL-2:

From one ampule containing in total 2×10⁶ IU human IFN-γ (2,000,000 IU; 0.1 mg) in 0.5 ml injection solution (injection solution comprising recombinant human IFN-γ-1b-protein comprising the peptide sequence SEQ ID NO: 1, produced by Boehringer Ingelheim RCV GmbH & Co KG and in Germany commercially available from Boehringer Ingelheim Pharma GmbH & Co. KG under the tradename Imukin®, PZN: 06958744, Lot: M000487), 0.5 ml (containing 2,000,000 IU; 0.1 mg IFN-γ) were mixed with 199.5 ml 0.9% NaCl solution (Braun Ecoflac Plus, B. Braun Melsungen AG, PZN: 08609255). This resulted in a concentration of 10,000 IU/ml and 500 ng/ml IFN-γ, respectively. From this solution, 100 ml were mixed with 899 ml 0.9% NaCl solution (Braun Ecoflac Plus, B. Braun Melsungen AG, PZN: 08609255) resulting in 999 ml solution containing IFN-γ.

Then, to one ampule containing 18×10⁶ IU (18,000,000 IU; 1.1 mg) lyophilized IL-2 (recombinant human IL-2-protein comprising the peptide sequence SEQ ID NO: 4, produced by Novartis Pharmaceuticals UK, Limited, and in Germany commercially available from Novartis Pharma GmbH under the tradename Proleukin®S) 9 ml water for injection (AQUA AD Injectabilia Miniplasco connect, B. Braun Melsungen AG, PZN 03113087) were added in order to solve the IL-2 to achieve a final concentration of 2×10⁶ IU/ml IL-2 (2,000,000 IU/ml; 122 μg/ml IL-2). Of this IL-2 solution, 1 ml was mixed with the above prepared 999 ml solution containing IFN-γ. This resulted in a final concentration of 1,000 IU/ml IFN-γ and 2,000 IU/ml IL-2, and 50 ng IFN-γ and 122 ng IL-2, respectively.

The solution was frozen in 0.5 ml aliquots in sterile glass ampules and stored at −20° C. until use.

Prior to administration, the thus obtained low dose IFN-γ/IL-2 injection solution of one glass ampule was thawed at room temperature and 0.1 ml (100 μl) were then injected into the skin of the patient corresponding to an amount of 100 IU IFN-γ and 200 IU IL-2, and ng IFN-γ and 12.2 ng IL-2, respectively.

8. Preparation of Injection Solution Comprising IL-4 and IL-2: IL-4/IL2 Injection Solution

To one ampule containing 10 μg (about 2.9×10⁵ IU, 290,000 IU) lyophilized IL-4 (recombinant human IL-4-protein comprising the peptide sequence SEQ ID NO: 3 carrier free, produced by R&D Systems and in Germany commercially available from R&D Systems, ordering number 204-IL-010/CF) 0.1 ml 0.9% NaCl solution (Braun Ecoflac Plus, B. Braun Melsungen AG, PZN: 08609255) were added and the IL-4 was reconstituted therein. The resulting solution was completely transferred to 49.85 ml 0.9% NaCl solution (Braun Ecoflac Plus, B. Braun Melsungen AG, PZN: 08609255) resulting in 49.95 ml solution containing IL-4.

Then, to one ampule containing 18×10⁶ IU (18,000,000 IU; 1.1 mg) lyophilized IL-2 (recombinant human IL-2-protein comprising the peptide sequence SEQ ID NO: 4, produced by Novartis Pharmaceuticals UK, Limited, and in Germany commercially available from Novartis Pharma GmbH under the tradename Proleukin®S) 9 ml water for injection (AQUA AD Injectabilia Miniplasco connect, B. Braun Melsungen AG, PZN 03113087) were added in order to solve the IL-2 to achieve a final concentration of 2×10⁶ IU/ml IL-2 (2,000,000 IU/ml; 122 μg/ml IL-2). Of this IL-2 solution, 0.05 ml were mixed with the above prepared 49.95 ml solution containing IL-4. This resulted in a final concentration of 0.2 μg/ml IL-4 and 2,000 IU/ml IL-2, and 0.2 μg/ml IL-4 and 122 ng/ml IL-2, respectively.

The solution was frozen in 0.5 ml aliquots in sterile glass ampules and stored at −20° C. until use.

Prior to administration, the thus obtained IL-4/IL-2 injection solution of one glass ampule was thawed at room temperature and 0.1 ml (100 μl) were then injected into the skin of the patient corresponding to an amount of 20 ng IL-4 and 200 IU IL-2, and 20 ng IL-4 and 12.2 ng IL-2, respectively.

9. Preparation of Injection Solution Comprising BDNF and IL-2: BDNF/IL2 Injection Solution

To one ampule containing 5 μg lyophilized BDNF (recombinant human BDNF-protein comprising the peptide sequence SEQ ID NO: 4, carrier free, produced by R&D Systems and in Germany commercially available from R&D Systems, ordering number 248-BD-005/CF) 0.1 ml 0.9% NaCl solution (Braun Ecoflac Plus, B. Braun Melsungen AG, PZN: 08609255) were added and the IL-4 was reconstituted. This resulting solution was completely transferred to 49.85 ml 0.9% NaCl solution (Braun Ecoflac Plus, B. Braun Melsungen AG, PZN: 08609255), resulting in 49.95 ml solution containing BDNF.

Then, to one ampule containing 18×10⁶ IU (18,000,000 IU; 1.1 mg) lyophilized IL-2 (recombinant human IL-2-protein comprising the peptide sequence SEQ ID NO: 4, produced by Novartis Pharmaceuticals UK, Limited, and in Germany commercially available from Novartis Pharma GmbH under the tradename Proleukin®S) 9 ml water for injection (AQUA AD Injectabilia Miniplasco connect, B. Braun Melsungen AG, PZN 03113087) were added in order to solve the IL-2 to achieve a final concentration of 2×10⁶ IU/ml IL-2 (2,000,000 IU/ml; 122 μg/ml IL-2). Of this IL-2 solution, 0.05 ml were mixed with the above prepared 49.95 ml solution containing BDNF. This resulted in a final concentration of 0.01 μg/ml BDNF and 2,000 IU/ml IL-2, and 0.01 μg/ml BDNF and 122 ng/ml IL-2, respectively.

The solution was frozen in 0.5 ml aliquots in sterile glass ampules and stored at −20° C. until use.

Prior to administration, the thus obtained BDNF/IL-2 injection solution of one glass ampule was thawed at room temperature and 0.1 ml (100 μl) were then injected into the skin of the patient corresponding to an amount of 1 ng BDNF and 200 IU IL-2, and 1 ng BDNF and 12.2 ng IL-2, respectively.

10. Preparation of Creme Comprising IFN-γ: IFN-γ Creme

From one ampule containing in total 2×10⁶ IU human IFN-γ (2,000,000 IU; 0.1 mg) in 0.5 ml injection solution (injection solution comprising recombinant human IFN-γ-1b-protein comprising the peptide sequence SEQ ID NO: 1, produced by Boehringer Ingelheim RCV GmbH & Co KG and in Germany commercially available from Boehringer Ingelheim Pharma GmbH & Co. KG under the tradename Imukin®, PZN: 06958744, Lot: M000487), 0.5 ml (containing 2,000,000 IU; 0.1 mg IFN-γ) were mixed with 94.5 g crème basis (Basiscrème DAC, Bombastus-Werke AG, PZN 04193119; density 9.5 g/10 ml) resulting in a crème comprising a concentration of 2,000 IU/100 μl IFN-γ.

Then, 1 ml of the IFN-γ crème were used twice daily for topical application in order to administer the IFN-γ topically to the skin.

11. Application of a Heat Pad—Administration of Conditioning Energy to the Skin

The skin was topically heated by placing a heat pad (latent heat storage “Relags Magic Heat Pad” by basic NATURE; size: 10 cm×10 cm; weight; 126 g; BSN350974) with slight pressure on the skin. Hence, the type of the heat pads employed in the Inventive Examples was the same as used in Reference Examples 1 and 3, except for Inventive Example 10, where temporarily an electrical hand warmer has also been employed. The heat pad develops according to the manufacturer's instructions a maximum temperature of 58° C. According to the measurements in, the used type of heat pad reached a maximum temperature of 49.4° C. (FIG. 6, Table 6). The heat pad was activated by pressing and clicking the integrated metal disc-activator. After solidification (crystallisation) and opacification of the gel-like contents of the heat pad were completed, the activated heat pad was immediately applied with slight pressure for 10 sec up to 5 min onto the subject's skin. After the heat pad was removed, the skin had noticeably warmed up, which could be determined by touching it with the hand and was palpable with the fingers. Furthermore, a clear redness of the skin had developed, wherein the redness was visually detectable to the naked eye.

Alternatively, an electrical hand warmer had been used as a heat pad in the same way as described above (Portable electric USB hand warmer and power bank including a temperature display, rechargeable, 10,000 mAh, manufactured by ReVolt, EAN: 4022107937498; or HONYIN hand warmer, rechargeable hand warmer, 7800 mAh, reusable, electric, power bank USB, portable). The ReVolt hand warmer was equipped with a display displaying the temperature. The temperature was adjusted between about 38° C. and 48° C. based on the temperature displayed in the display. The HONYIN had warmer was either used at the low temperature or the medium temperature. According to the manufacturer's indications the low temperature is about 35° C. to 42° C. (95 F to 108 F), the medium temperature about 42° C. to 48° C. (108 F to 118 F).

12. Application of Heat Creme—Administration of a Skin-Conditioning Agent to the Skin

As a heat crème Kytta® heat balm containing methylnicotinat (“Kytta® Warmebalsam” by P&G Health Germany GmbH, Germany, PZN 12358936) was applied topically to the skin of a subject. Hence, the heat crème employed in the Inventive Examples was the same as used in Reference Examples 2 and 4. The dimensions of the area, to which the heat crème was applied, was in the range of 4 cm² to 100 cm². About 10 mg to 800 mg, usually about 35 mg to 150 mg, heat crème were administered to the skin of the skin area by applying a thin film of heat crème thereto. Subsequently, the treated skin became noticeably warm within about 15 minutes. The warming could be determined by touching the treated skin with the hand or fingers. Furthermore, in case of a Caucasian skin, a clear redness of the skin developed, wherein the redness was visually detectable to the naked eye.

A detailed description of the effect of methylnicotinate on elevated perfusion can be found in Tur E et al. (Tur E, Guy RH, Tur M, Maibach HI. “Noninvasive assessment of local nicotinate pharmacodynamics by photoplethysmography”. J Invest Dermatol. 1983 June; 80(6):499-503).

The local pharmacodynamics of a topical vasodilator (methyl nicotinate (MN) has been described by Elawa et al. (Elawa S, Mirdell R, Farnebo S, Tesselaar E. “Skin blood flow response to topically applied methyl nicotinate: Possible mechanisms”. Skin Res Technol. 2020 May; 26(3):343-348. Epub 2019 Nov. 27. PMID: 31777124.). Particularly, the authors described that a dose of 50 μl of 20 mmol/l

MN results in a reproducible perfusion response in healthy subjects using a Laser Speckle Contrast Imager (PeriCam PSI System; Perimed AB) was used to measure the perfusion of the skin. The perfusion increase occurs a few minutes after topical application of MN and reaches a stable plateau phase after five minutes.

13. Determination of Joint Condition—Tender Joints (TJ) and Swollen Joints (SJ)

The investigation and determination of the joint condition has been performed by a physician or rheumatologist. A tender joint (TJ) was pressure sensitive but not necessarily swollen. A swollen joint (SJ) shows a swelling, which was palpable by manual investigation and/or visually detectable to the naked eye. The amount of TJ and SJ was counted. A reduction in the amounts of TJ and/or SJ, respectively, was an indication for a reduction in inflammation, mitigation of the disease and an improvement of the patient's health condition.

14. CRP—C-Reactive Protein

The CRP (C-reactive protein) is the most important blood laboratory value for detecting and monitoring inflammation in the body. It was determined by a certified laboratory. A low CRP indicates the absence of an inflammation, while a high CRP is indicative for an inflammation to be present.

It is a highly sensitive marker. Already a tiny focus of inflammation may already cause a rise of the CRP. Furthermore, it is known that elevated CRP-values show an association with mental as well as physical stress. Hence, an increased or fluctuating CRP may also be due to mental stress like depression or concerns and physical stress like physical strain or physical labor. Such increase may override the positive effects on the CRP achieved by the present invention and the CRP development in such patients may then no longer be indicative for disease control of the inflammatory disease, immunological disease and/or autoimmunological disease. Hence, a fluctuating or increased CRP does not exclude the effectiveness of the present invention. However, a low or decreased CRP is a clear indication for lack of any inflammations in the patient's body and disease control.

TABLE 1
Scoring of CRP-ranges for an adult human:

| | CRP |
|---|---|
| Score | [mg/l {milligram/litre}] |
| normal | 0 to 5 |
| slightly increased | >5 to 40 |
| moderately increased | >40 to 100 |
| strongly increased | >100 |

15. BASDAI—Bath Ankylosing Spondylitis Disease Activity Index Disease Activity Questionnaire

The BASDAI (Bath Ankylosing Spondylitis Disease Activity Index) is a validated and standardized diagnostic questionnaire which allows a physician, usually a rheumatologist, to determine the effectiveness of a current drug therapy, or the need to institute a new drug therapy for the treatment of ankylosing spondylitis (Bechterew's disease). A higher BASDAI indicates a higher level of suffering pressure and suboptimal disease control in the patient, while a lower BASDAI indicates an improved life quality of the patient due to better disease control.

The BASDAI consists of a 0 to 10 scale measuring discomfort, pain, and fatigue (0 being no problem and 10 being the worst problem) in response to six questions asked of the patient pertaining to the five major symptoms of ankylosing spondylitis:
1) Fatigue
2) Spinal pain
3) Arthralgia (joint pain; tender joints) or swelling;
4) Enthesitis or inflammation of tendons and ligaments (areas of localized tenderness where connective tissues insert into bone);
5) Morning stiffness duration;
6) Morning stiffness severity.

To give each symptom equal weighting, the average of the two scores relating to morning stiffness [5) and 6)]) was taken. The resulting 0 to 50 score was divided by 5 to give a final 0 to 10 BASDAI-score. Scores of 4 or greater suggest suboptimal control of disease. Hence, those patients are good candidates for testing the method according to present invention.

16. HAQ—Health Assessment Questionnaire Functional Disability Index

The HAQ (Health assessment Questionnaire) is a standardized questionnaire to assess disability due to inflammatory rheumatic joint disease like polyarthritis, rheumatoid arthritis, psoriatic arthritis, polymyalgia rheumatic etc. Patients completed this questionnaire themselves, assessing their ability to perform the following activities in eight daily functional domains: Dressing, getting up, eating, walking, personal hygiene, handing objects, grasping, other activities.

On a scale of 0 to 3, a distinction is made as to whether the activities can be performed:
Scale value 0=no disability, can be performed without ANY difficulty;
Scale value 1=can be performed with SOME difficulty;
Scale value 2=can be performed with MUCH difficulty;
Scale value 3=high disability, UNABLE to do.

Furthermore, each of the disability items on the HAQ has a companion aids/devices variable that is used to record what type(s) of assistance, if any, the participant uses for his/her usual activities. These variables (see below) are coded as follows:
Scale value 0=no assistance is needed.
Scale value 1=a special device is used by the patient in his/her usual activities.
Scale value 2=the patient usually needs help from another person.
Scale value 3=the patient usually needs BOTH a special device AND help from another person.

The functional disability index (HAQ) is the average of the highest numerical values reported for each of the eight different domains. High HAQ-scores correspond to a high degree of disability.

A change of 0.22 units in the HAQ-score is generally considered to be the smallest distinguishable unit (minimum clinically distinguishable unit). A clinically significant improvement is considered to be a decrease in HAQ of at least 0.5 units.

A decrease in the HAQ of 0.5 score-points or more is regarded as significant based on the publications of Strand V et al (c.f. particularly, the calculation in Table 1, Strand V, van Vollenhoven R F, Lee E B, Fleischmann R, Zwillich S H, Gruben D, et al. “Tofacitinib or adalimumab versus placebo: patient-reported outcomes from a phase 3 study of active rheumatoid arthritis”. Rheumatology (Oxford), 2016 June; 55(6):1031-41, doi: 10.1093/rheumatology/kev442; and Strand V, Kremer J, Wallenstein G, Kanik KS, Connell C, Gruben D, et al. “Effects of tofacitinib monotherapy on patient-reported outcomes in a randomized phase 3 study of patients with active rheumatoid arthritis and inadequate response to DMARDs”. Arthritis Res Ther. 2015; 17:307. doi: 10.1186/s13075-015-0825-9)

17. SHILD—Questionnaire for Multiple Sclerosis

The SHILD (for German: Gesundheitsbezogene Selbsthilfe in Deutschland—Entwicklungen, Wirkungen, Perspektiven) provides a questionnaire created in the framework of a multicenter study funded by the German Federal Ministry of Health on the state of health-related self-help in Germany. Further information can be found at https://www.uke.de/extern/shild/.

For the purposes of the present invention, the questionnaire of the SHILD study for the indication group of multiple sclerosis was used for data collection, wherein only the section “C—Health-related quality of life” was evaluated. Said section C divided into four sub-sections:
C1—consists of one question: “flow would you describe your health condition in general?”
C2—contains 22 question addressing several areas of health.
C3—consists of one question: “flow would you rate your overall quality of life over the past four weeks?”
C4—contains 12 statements that relate to the patient's condition and possible concerns about the future.

The particular questions and statements of section C of the SHILD were as follows. The answers possible and given by the SHILD-questionnaire are also indicated in the following. Patients were asked to tick off exactly one of the given answers what applies best.
As regards sub-section C1:
   Question: How would you describe your state of health in general?
   Possible answers given by SHIED: very good, good, moderate, poor or very poor
As regards sub-section C2:
   Question: In the last 4 weeks, how often did you have due to your MS . . . :
      (1) . . . difficulty going around outside the house?; (2) . . . difficulties with activities outside the home, i.e. shopping, going to the cinema . . . ?; (3) . . . difficulty walking around at home?; (4) . . . balance or walking problems?; (5) . . . difficulties with leisure activities at home, i.e. DIY, gardening . . . ?; (6) . . . difficulties with activities in the workplace, i.e. integration, interruptions, restrictions . . . ?; (7) . . . quickly feel signs of fatigue?; (8) . . . the feeling of having no energy?; (9) . . . vision problems: Do you see worse or is your vision disturbed in any way?; (10) . . . feel unpleasant sensations, i.e. hot or cold feeling . . . ?; (11) . . . a compromised love life?; (12) . . . a compromised sex life?; (13) . . . felt offended by other people's looks?; (14) felt unsafe in public?; (15) . . . anxious?; (16) . . . depressed or dispressed?; (17) . . . close to tears?; (18) . . . nervous or annoyed about individual matters or situations?; (19) . . . burdened by a decline in your memory capacity?; (20) . . . unfocused, e.g. when reading, watching a film or following a conversation?; (21) . . . exposed to an above-average urge to urinate?; and (22) . . . plagued by problems with your bladder control?
   Possible answers: never, rare, sometimes, often or always
As regards sub-section C3:
   Question: How would you rate your overall quality of life over the last four weeks?
   Possible answers: very good, good, moderate, poor or very poor
As regards sub-section C4:
   Statements relating to the patient's condition and possible concerns about the future:
      (1) When I think about the further course of my illness, I get scared. (2) I get really nervous before doctor's appointments or check-ups; (3) I am afraid of pain; (4) The thought that I might no longer be to perform as well at work scares me; (5) When I feel anxious, I also feel it physically (e.g. palpitations, stomach ache, tension); (6) The question of whether my children could also get my disease worries me; (7) It worries me that I could be dependent on outside help in everyday life; (8) I'm worried that I won't be able to pursue my hobbies anymore because of my illness; (9) I am afraid of drastic medical measures in the course of the disease; (10) I am worried that my medication could harm my body; (11) I am worried about what will happen to my family if something should happen to me; and (12) The thought that I could be absent from work because of MS worries me.
   Possible answers: never, rare, sometimes, often, very often or does not apply to me

Every answer to a question/statement was assigned a value for the purposes of the present invention, if it did not match the categories of the assigned values from the outset. This assignment was done for easier comparison of the patient's statements. The assignment was as follows:

TABLE 2
Answer and correspondingly assigned value used for the SHILD-scoring

| Score | 0 | 1 | 2 | 3 | 4 | (no score assigned) |
|---|---|---|---|---|---|---|
| Answer: | never | rare | sometimes | often | always/very often | does not apply to me |
| Assigned value | very good | good | moderate | poor | very poor | (not evaluated) |

Additionally, for sub-sections C2 and C4, a ratio was calculated by summing up the values of all answers divided by the number of questions. The ratio is indicated as ‘impact on quality of life’ and ‘impact on psychophysics’.

It is noted that the questions, statements and answers are translated from German. The exact original wording of the SHIED-questionnaire can be found at file: https://www.google.com/url?sa=t&rct=j&q=&esrc=s&source=web&cd=&ved=2ahUKEwislI3TOuf6AhVLQPEDHZHcCjIQFnoEC BYQAQ&url=https %3A %2F %2Fwww.uke.de %2Fextern %2Fshild %2FMaterialien Dateien %2FFragebogen SHIED MS final.pdf &usg=AOvVaw 1-bZX4kqeeXMBEFtSr1Mkf

The invention will be explained in more detail by the following Inventive Examples, Reference Examples and Comparative Examples. The examples are only for illustrative purposes and shall not limit the invention thereto.

### EXAMPLES

Beforehand, a brief explanation of the abbreviations is given for a better understanding which abbreviations are used in Reference Examples 1 to 4:
The capital letter “T” stands for point in time.
The lower case “b” indicates a blood measurement within the skin using O2C measurements.
The lower case “t” indicates a temperature measurement on the skin using the thermal measurements.
The lower case “p” indicates a measurement when a heat pad was used.
The lower case “c” indicates a measurement when a heat crème was used.
The letters or numbers in the index closer specify the individual point in time.

Hence, for instance T(bp)₁ stands for point in time 1 regarding a blood measurement within the skin using a heat pad.

### Reference Example 1 (RE 1): O2C-Measurement on a Subject when Using a Heat Pad

A female person (51 years of age) volunteered for the O2C measurement using a heat pad as a subject to be tested. The subject was normotensive and nonsmoker. The measurement was performed as described in the Materials and Methods' section ‘O2C—Oxygen to See’.

Blood pressure and pulse were monitored and did not change over the entire measurement.

After the resting time of 15 min to achieve acclimatisation to the environment and thereby ensure stable conditions, the baseline measurement was conducted to get starting values for the sO₂, rHb, rFlow and Vel indicated at point in time T(bp)_{base}. The values are indicated in Table 3.

Directly after finishing the baseline measurement, the measurement using the heat pad was conducted as described in the Materials and Methods' section ‘O2C—Oxygen to See’, item (C).

At point in time T(bp)₁, which was 60 sec (1 min) after removing the heat pad from M4, the determination of the values for the sO₂, rHb, rFlow and Vel were started and continued until the baseline levels had re-established. The values for the sO₂, rHb, rFlow and Vel have been obtained for points in time T(bp)_{base} and T(bp)₁ to T(bp)₁₂. The timing of the measurements, including the points in time and the intervening time intervals, are described in Table 3.

The subject did not feel any sensations of pain during the entire measurement, neither during the rise in temperature, during the application of the hat pad nor in respect to the reddening of the skin, particularly not on and around measurement area M4.

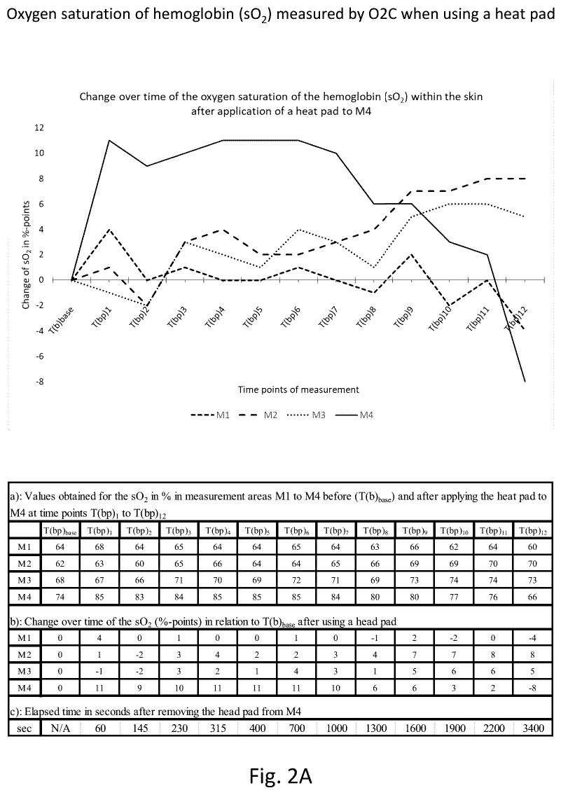

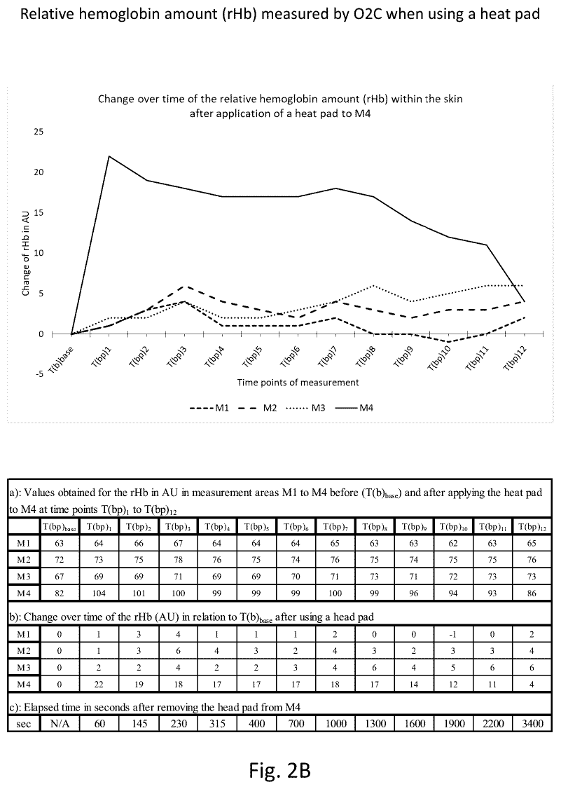

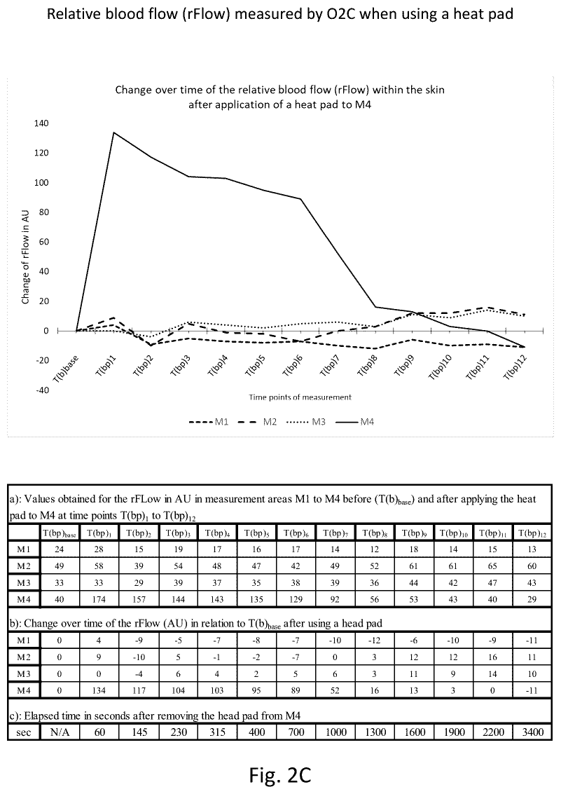

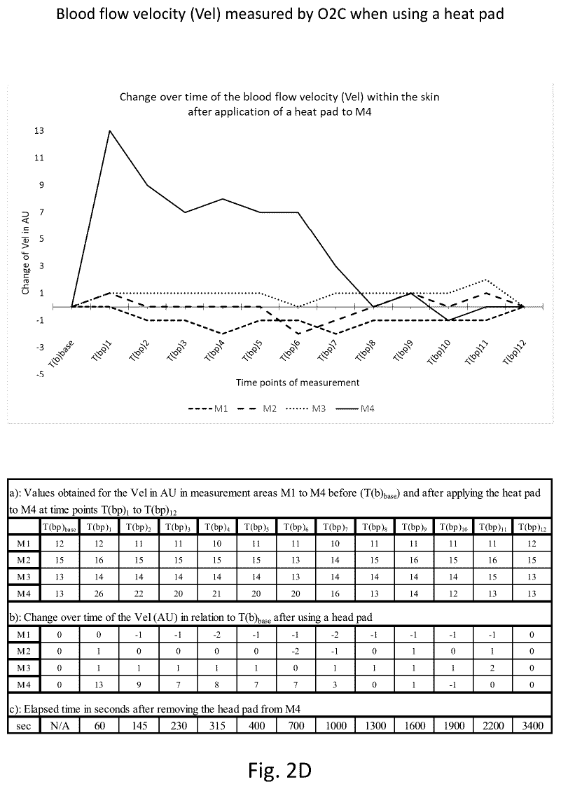

In FIG. 2A, the upper table indicates the values obtained for the sO₂ in %; the lower table indicates the amount of change in the sO₂ in percentage point (% points) over time in respect to the value obtained at T(bp)_{base} (determined by subtraction); and in the diagram the amount of change in the sO₂ was plotted against points in time T(bp)_{base} and T(bp)₁ to T(bp)₁₂.

In FIG. 2B, the upper table indicates the values obtained for the rHb in AU; the lower table indicates the amount of change of the rHb in AU over time in respect to the value obtained at T(bp)_{base} (determined by subtraction); and in the diagram the amount of change in the rHb was plotted against points in time T(bp)_{base} and T(bp)₁ to T(bp)₁₂.

In FIG. 2C, the upper table indicates the values obtained for the rFlow in AU; the lower table indicates the amount of change of the rFlow in AU over time in respect to the value obtained at T(bp)_{base} (determined by subtraction); and in the diagram the amount of change in the rFlow was plotted against points in time T(bp)_{base} and T(bp)₁ to T(bp)₁₂.

In FIG. 2D, the upper table indicates the values obtained for the Vel in AU; the lower table indicates the amount of change of the Vel in AU over time in respect to the value obtained at T(bp)_{base} (determined by subtraction); and in the diagram the amount of change in the Vel was plotted against points in time T(bp)_{base} and T(bp)₁ to T(bp)₁₂.

TABLE 3
Description of points in time of the O2C-measurement
of the skin surface temperature when using a heat pad

| Points in time | | |
|---|---|---|
| and intervening | | |
| time intervals | Description | |
| T(bp)_{base} | initial state before the application of the heat pad | |
| T(bp)₁ | 60 sec (1 min) after removing the heat pad from M4 | |
| Time interval | 85 sec | (1.4 min) |
| T(bp)₂ | 145 sec (2.4 min) after removing the heat pad from M4 | |
| Time interval | 85 sec | (1.4 min) |
| T(bp)₃ | 230 sec (3.8 min) after removing the heat pad from M4 | |
| Time interval | 85 sec | (1.4 min) |
| T(bp)₄ | 315 sec (5.3 min) after removing the heat pad from M4 | |
| Time interval | 85 sec | (1.4 min) |
| T(bp)₅ | 400 sec (6.7 min) after removing the heat pad from M4 | |
| Time interval | 300 sec | (5 min) |
| T(bp)₆ | 700 sec (11.7 min) after removing the heat pad from M4 | |
| Time interval | 300 sec | (5 min) |
| T(bp)₇ | 1000 sec (16.7 min) after removing the heat pad from M4 | |
| Time interval | 300 sec | (5 min) |
| T(bp)₈ | 1300 sec (21.7 min) after removing the heat pad from M4 | |
| Time interval | 300 sec | (5 min) |
| T(bp)₉ | 1600 sec (26.7 min) after removing the heat pad from M4 | |
| Time interval | 300 sec | (5 min) |
| T(bp)₁₀ | 1900 sec (31.7 min) after removing the heat pad from M4 | |
| Time interval | 300 sec | (5 min) |
| T(bp)₁₁ | 2200 sec (36.7 min) after removing the heat pad from M4 | |
| Time interval | 1200 sec | (20 min) |

| | |
|---|---|
| T(bp)₁₂ | 3400 sec (56.7 min) after removing the heat pad from M4 |

T(bp)_{base} and T(bp)₁ to T(bp)₁₂ correspond to T(bp)_{base} and T(bp)₁ to T(bp)12, respectively, of FIGS. 2A to 2D.

### Reference Example 2 (RE 2): O2C-Measurement on a Subject when Using a Heat Crème

Same female person as stated in Reference Example 1 volunteered for the O2C measurement using heat crème as a subject to be tested. The measurement was performed as described in the Materials and Methods' section ‘O2C—Oxygen to See’.

After finishing the measurement stated in Reference Example 1, and following a break of 20 min (about 75 min after the baseline measurement at T(bc)_{base}), the measurement using a heat crème was conducted as described in the Materials and Methods' section ‘O2C—Oxygen to See’, item (D).

Blood pressure and pulse were monitored and did not change over the entire measurement and in respect to the measurement of Reference Example 1.

At point in time T(bc)₁, which was 120 sec (2 min) after finishing the application of the Kytta® heat balm to M1, the determination of the values for the sO₂, rHb, rFlow and Vel were started and continued until for the sO₂, rFlow and Vel baseline levels had re-established. The values for the sO₂, rHb, rFlow and Vel have been obtained for points in time T(bc)_{base} and T(bc)₁ to T(bc)₁₃. The timing of the measurements, including the points in time and the intervening time intervals, is described in Table 4. The subject did not feel any sensations of pain during the entire measurement, neither during the rise in temperature, during the application of the test and control compositions nor in respect of the reddening of the skin, particularly not on and around measurement area M1.

In FIG. 3A, the upper table indicates the values obtained for the sO₂ in %; the lower table indicates the amount of change in the sO₂ in percentage point (% points) over time in respect to the value obtained at T(bc)_{base} (determined by subtraction); and in the diagram the amount of change in the sO₂ was plotted against points in time T(bc)_{base} and T(bc)₁ to T(bc)₁₃.

In FIG. 3B, the upper table indicates the values obtained for the rHb in AU; the lower table indicates the amount of change of the rHb in AU over time in respect to the value obtained at T(bc)_{base} (determined by subtraction); and in the diagram the amount of change in the rHb was plotted against points in time T(bc)_{base} and T(bc)₁ to T(bc)₁₃.

In FIG. 3C, the upper table indicates the values obtained for the rFlow in AU; the lower table indicates the amount of change of the rFlow in AU over time in respect to the value obtained at T(bc)_{base} (determined by subtraction); and in the diagram the amount of change in the rFlow was plotted against points in time T(bc)_{base} and T(bc)₁ to T(bc)₁₂.

In FIG. 3D, the upper table indicates the values obtained for the Vel in AU; the lower table indicates the amount of change of the Vel in AU over time in respect to the value obtained at T(bc)_{base} (determined by subtraction); and in the diagram the amount of change in the Vel was plotted against points in time T(bc)_{base} and T(bc)₁ to T(bc)₁₂.

TABLE 4
Description of points in time of the O2C-measurement
of the skin surface temperature when using heat creme

| Points in time | | |
|---|---|---|
| and intervening | | |
| time intervals | Description | |
| T(bc)_{base} | initial state before the application of the heat crème | |
| T(bc)₁ | 120 sec (2 min) after finishing the application of the heat crème to M1 | |
| Time interval | 85 sec | (1.4 min) |
| T(bc)₂ | 205 sec (3.4 min) after finishing the application of the heat crème to M1 | |
| Time interval | 85 sec | (1.4 min) |
| T(bc)₃ | 290 sec (4.8 min) after finishing the application of the heat crème to M1 | |
| Time interval | 145 sec | (2.4 min) |
| T(bc)₄ | 435 sec (7.25 min) after finishing the application of the heat crème to M1 | |
| Time interval | 145 sec | (2.4 min) |
| T(bc)₅ | 580 sec (9.7 min) after finishing the application of the heat crème to M1 | |
| Time interval | 300 sec | (5 min) |
| T(bc)₆ | 880 sec (14.7 min) after finishing the application of the heat crème to M1 | |
| Time interval | 300 sec | (5 min) |
| T(bc)₇ | 1180 sec (19.7 min) after finishing the application of the heat crème to M1 | |
| Time interval | 600 sec | (10 min) |
| T(bc)₈ | 1780 sec (29.7 min) after finishing the application of the heat crème to M1 | |
| Time interval | 600 sec | (10 min) |
| T(bc)₉ | 2380 sec (39.7 min) after finishing the application of the heat crème to M1 | |
| Time interval | 600 sec | (10 min) |
| T(bc)₁₀ | 2980 sec (49.7 min) after finishing the application of the heat crème to M1 | |
| Time interval | 600 sec | (10 min) |
| T(bc)₁₁ | 3580 sec (59.7 min) after finishing the application of the heat crème to M1 | |
| Time interval | 600 sec | (10 min) |
| T(bc)₁₂ | 4180 sec (69.7 min) after finishing the application of the heat crème to M1 | |
| Time interval | 600 sec | (10 min) |

| | |
|---|---|
| T(bc)₁₃ | 4780 sec (79.7 min) after finishing the application of the heat crème to M1 |

T(bc)_{base} and T(bc)₁ to T(bc)₁₃ correspond to T(bc)_{base} and T(bc)₁ to T(bc)₁₃, respectively, of FIGS. 3A to 3D.

### Reference Example 3 (RE 3): Thermal Measurement (Skin Surface Temperature) and Skin Reddening on a Subject when Using a Heat Pad

A female person (51 years of age) volunteered for the thermal measurement and skin reddening using a heat pad as subject to be tested. The subject was normotensive and nonsmoker. The measurement was performed as described in the Materials and Methods' section ‘Thermal measurement and quantification of skin reddening’.

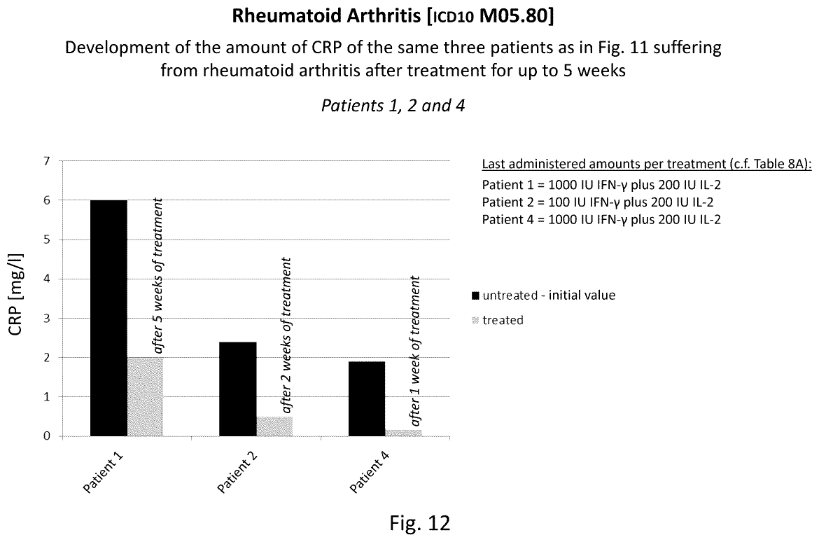

To ensure stable measurement conditions, the skin surface temperature in all 4 measurement areas, M1 to M4, was recorded for 20 min to serve as a baseline (FIG. 12). A slow but very stable decrease in the temperature of approximately 0.5° C. within a 20 min timeframe was shown indicating stable measurement conditions. The slow but very stable decrease in the temperature was presumably due to reduced body activity. As an example, the temperature readings after approximately 10.8 min (T(t)_{base}) in the 4 measurement areas, M1 to M4, were selected (FIG. 5) and are stated in the following Table 5.

TABLE 5
Baseline skin surface temperature at measurement
areas M1 to M4 at point in time T(t)_{base}
Temperature after 10.8 min = T(t)_{base}

| | |
|---|---|
| M1 | 29.6° C. |
| M2 | 29.2° C. |
| M3 | 29.2° C. |
| M4 | 29.5° C. |

Hence, the average of the baseline skin surface temperature of M1 to M4 was 29.4° C.

Twenty min after finishing the baseline measurement, the measurement using the heat pad was conducted as described in the Materials and Methods' section ‘Thermal measurement and quantification of skin reddening’, item (C).

Ten seconds after the heat pad was removed from M4, the start of the measurements of the skin surface temperature on all measurement areas M1 to M4 without heat pad on M4 was indicated at point in time T(tp)₁ (FIG. 6, Table 6).

The starting temperature of the skin surface for heat pad treated M4 was 43.9° C. at T(tp)₁ and was, hence, clearly increased above the baseline skin surface temperature (average of 29.4° C., Table 6) and the average starting temperature of the skin surface of the untreated controls, M1 to M3 (average of M1 to M3 of 29.4° C. at T(tp)₁, Table 6). Hence, the starting temperatures of the skin surfaces on the untreated controls, M1 to M3, were at T(tp)₁ were approximately the same as the baseline skin surface temperature. The subsequent temperature development on all 4 measurement areas, M1 to M4, was continuously measured every second for about 59 min (FIG. 6) until the difference between the untreated controls, M1 to M3 (average of M1 to M3 of 29.5° C., at T(tp)₃) and the heat pad treated M4 (30.8° C. at T(tp)₃) was about 1.3° C. (a level approximately 1.4° C. above the average of the baseline skin surface temperature) (FIG. 6, Table 6). With respect to time, the measured skin surface temperature on heat pad treated M4 was gradually decreasing, while the skin surface temperatures on the untreated controls M1 to M3 remained constant around the average of the baseline temperature of M1 to M4 and the average of the starting temperature of M1 to M3 over the entire measurement period of 59 min (FIG. 6, Table 6).

After 57 min, a slight reddening of the skin was still visible at and around heat pad treated M4. The untreated control M3 located on the same left forearm as M4 and both controls, M1 and M2, on the right forearm, were unaffected, showing that the treatment with the heat pad was a locally limited phenomenon.

The temperature difference between the skin surface at and around M4 and the skin surface of untreated skin, particularly at M1 to M3, was clearly palpable with the fingers.

The subject did not feel any sensations of pain during the entire measurement, neither during the rise in temperature, during the application of the heat pad nor in respect of the reddening of the skin, particularly not on and around M4.

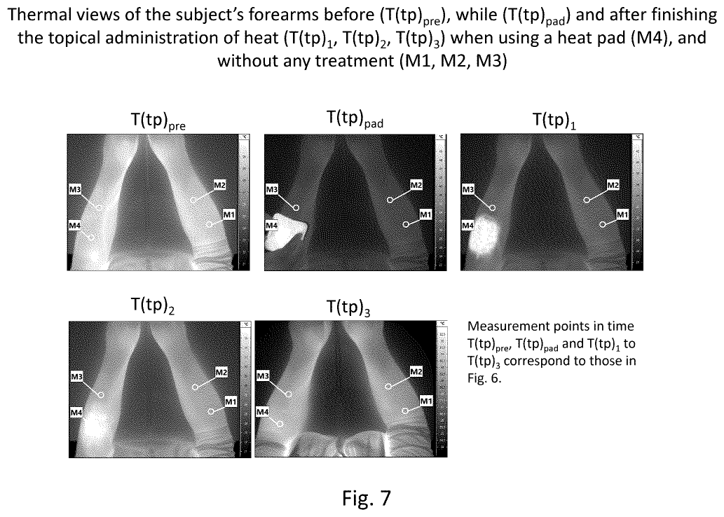

It can be seen from all thermal views in FIG. 7 that the skin surface temperature shown for M1 to M3 was similar for the entire forearm.

Furthermore, on the thermal views at T(tp)₁ and T(tp)₂ it can be seen that the areal actually heated at and around M4 (corresponds to the skin area according to the present invention) was larger than the area on which the heat pad was applied (corresponds to the application area according to the present invention). This may be because the heat radiates into the adjacent skin areals and presumably because the heat supply causes capillaries in adjacent skin areals to dilate, indicated by a temperature increase on the skin surface beyond the areal heated by the heat pad. Hence, in this example, the application area was the areal the heat pad was topically applied to. The skin area was the areal finally heated that is the areal to which heat was administered.

TABLE 6
Skin surface temperatures on M1 to M3 and temperature
of the heat pad on M4 at T(tp)_{pad}; skin surface temperatures on
M1 to M4 at T(tp)₁, T(tp)₂ and T(tp)₃, respectively,
in accordance with FIGS. 6 and 7.

| | T(tp)ₚᵣₑ | T(tp)_{pad} | T(tp)₁ | T(tp)₂ | T(tp)₃ |
|---|---|---|---|---|---|
| | [° C.] * | [° C.] * | [° C.] ^{#} | [° C.] ^{#} | [° C.] ^{#} |
| M1 (untreated control) | 29.2 | 29.2 | 29.2 | 29.1 | 29.4 |
| M2 (untreated control) | 29.2 | 29.2 | 29.2 | 29.2 | 29.5 |
| M3 (untreated control) | 29.6 | 29.7 | 29.6 | 29.6 | 29.7 |
| M4 (heat pad | 29.5 | 49.4 | 43.9 | 34.3 | 30.8 |
| application) | | | | | |

| Point in time | Description |
|---|---|
| T(tp)_{pad} | during the application of the heat pad. * |
| T(tp)₁ | 1 sec (0.02 min) after removal of the heat pad from M4. ^{#} |
| T(tp)₂ | 300 sec (5 min) after removal of the heat pad from M4. ^{#} |
| T(tp)₃ | 3420 sec (57 min) after removal of the heat pad from M4. ^{#} |

* Point in time T(tp)_{pad} is, as indicated above, during the application of the heat pad on M4. Hence, it is noted that at T(tp)_{pad} on M4 the temperature of the heat pad surface was measured and recorded on the thermal views while on M1 to M3 the surface temperature of the skin.
^{#} Points in time T(tp)₁ to T(tp)₃ are, as indicated above, after the removal of the heat pad from M4. Hence, on M1, M2, M3 and M4 the surface temperature of the skin was measured and recorded on the thermal views.
T(tp)_{pad}, T(tp)₁, T(tp)₂ and T(tp)₃ correspond to T(tp)_{pad}, T(tp)₁, T(tp)₂ and T(tp)₃, respectively, of FIGS. 6 and 7.

### Reference Example 4 (RE 4): Thermal Measurement (Skin Surface Temperature) and Skin Reddening on a Subject when Using Heat Crème

Same female person as stated in Reference Example 3 volunteered for the thermal measurement and skin reddening using heat crème as subject to be tested. The measurement was performed as described in the Materials and Methods' section ‘Thermal measurement and quantification of skin reddening’.

After finishing the measurement stated in Reference Example 3, a one hour break was taken. Then the measurement using the heat pad was conducted as described in the Materials and Methods' section ‘Thermal measurement and quantification of skin reddening’, item (D). It can be seen that the baseline skin surface temperatures at measurement areas M1 to M4 and the average baseline temperature (Table 5) are very similar or even identical to the respective starting temperatures of the measurements using a heat pad and a heat crème (Tables 6 and 7), respectively. This indicates that the measurement conditions remained constant.

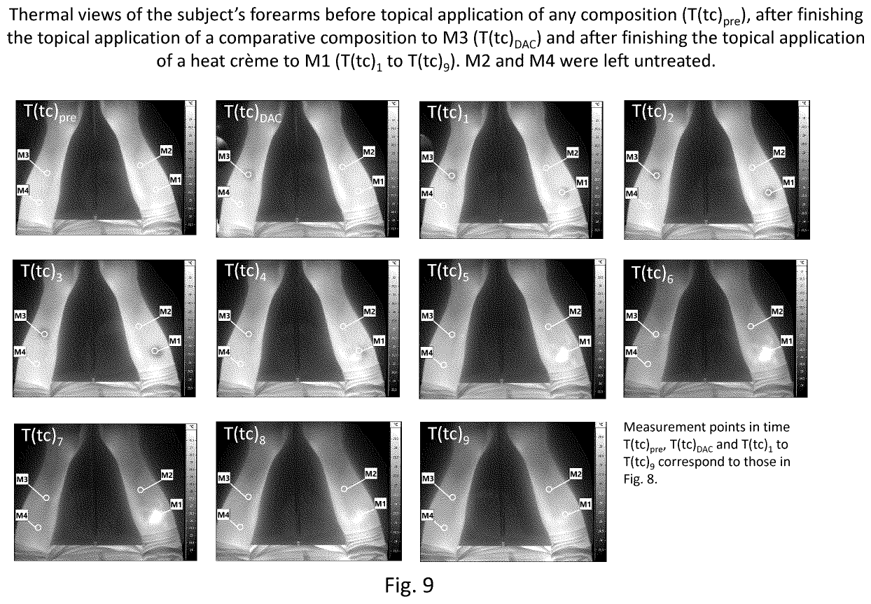

Before applying the compositions to the skin, the measurements of the skin surface temperatures of M1 to M4 were started and continuously measured every second for 110 min (until about T(tc)₉, FIGS. 8 and 9, Table 7) until the temperature difference between M3 (comparative composition) on the left forearm and M1 (test composition) on the right forearm reached a level of about 1.8° C.

The starting temperature measured for the skin surface on M2 and M3, both located at the distal positions on the forearms, at T(tc)ₚᵣₑ (before starting the application of the compositions), was very similar (29.4° C. and 29.2° C., which was around the average of baseline skin surface temperature of 29.4° C., Table 5). The temperature measured for the skin surface on M3 at T(tc)_{DAC}, which was 1 sec after the application of the comparative composition to M3 was finished, was 28.1° C. (FIG. 8, Table 7).

M1 and M4 are both located at the proximal positions on the forearms. The starting temperature measured for the skin surface on M1 and M4 at T(tc)ₚᵣₑ and T(tc)_{DAC} (both before the application of the Kytta® heat balm) was very similar with 30.1° C. each for M1, and 30.0° C. and 30.1° C. for M4 (Table 7) (which was slightly higher than the average of baseline skin surface temperature of 29.4° C., Table 5). The temperature measured for the skin surface on M1 at T(tc)₁, which was 10 sec after the application of the test composition to M1 was finished (and 60 sec after the application of the comparative composition to M3 was finished), was 29.2° C. (FIG. 8, Table 7).

As can be seen from FIGS. 8 and 9, after the application of the test composition, the measured values of the skin surface temperature decreased with time in M1 from 30.1° C. at T(tc)ₚᵣₑ and T(t)_{DAC} to a minimum of 27.9° C. at T(tc)₃, presumably due to evaporative cooling of the test composition's moisture and/or due to a transient change of the skin's emissivity (8) which may have caused the measured values to vary from reality (Jr, George & Barter, Archie. (2006). Dual-Band MWIR/LWIR Radiometer for absolute temperature measurements. Proceedings of SPIE—The International Society for Optical Engineering. 6205. 10.1117/12.668666 Chapter 5.4.). Likewise, the skin surface temperature decreased with time in M3 from 29.2° C. at T(tc)ₚᵣₑ to a minimum of 27.4° C. at T(tc)₂ after the application of the comparative composition. The temperature minimum of the comparative composition at M3 was with

T(tc)₂ about 1.2 min earlier than the temperature minimum of the test composition at M1 with T(tc)₃, which was in accordance with the time shift of about one minute the comparative composition was applied earlier (FIG. 8, Table 7).

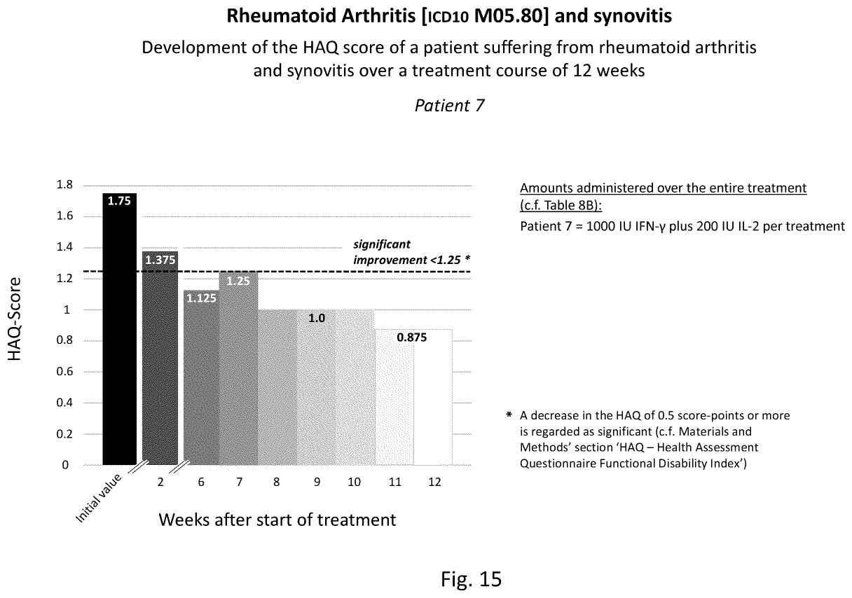

After the temperature minimum at T(tc)₃ the rise of the skin surface temperature in M1 was steeper than for the comparative composition at M3 after the temperature minimum at T(tc)₂ (FIG. 8). At point in time T(tc)₄ the skin surface temperature of both, M1 and M3, reached the starting temperatures of before the application of the test composition and the comparative composition, respectively, at T(tc)ₚᵣₑ. After T(tc)₄, the skin surface temperature for the test composition in M1 rapidly exceeded the starting temperature/baseline temperature. At T(tc)₅ the temperature in M1 was with 31.4° C., which was clearly higher than the starting temperature of 30.1° C. and also clearly higher than the skin surface temperature 28.5° C. for the comparative composition in M3 at T(tc)₅. Only for the test composition an increase in the skin surface temperature to 32.6° C. was measured (M1 at (T(tc)₆), which was approximately the maximum skin surface temperature reached with the test composition. The maximum skin surface temperature was maintained in M1 for about 15 min until T(tc)₇ (FIG. 8). Subsequently, the temperature in M1 slowly decreased. At T(tc)₈ the temperature in M1 was decreased to 29.8° C. and still above the skin surface temperature of M1 to M3 (27.4° C. to 28.3° C.) but below the starting temperature of 30.1° C. At about T(tc)₉, the skin surface temperature in M1 finally had returned to about the starting temperature (FIG. 15) and the difference to untreated control M4 reached a level of about 1° C. (FIG. 15, Table 7). M4 is like M1 located on the proximal position on the forearm and, hence, both position as well comparable.

The Basiscrème DAC applied on the skin of M3 may have caused a reduced heat loss and thus a slight increase in the skin temperature between about T(tc)₄ and T(tc)₇. Nevertheless, for the comparative composition in M3 no increase above the average of the baseline skin surface temperature of 29.4° C. as well as the starting temperatures at M2 and M3 at T(tc)ₚᵣₑ could be measured over the entire measurement period (FIGS. 5, 8 and 9, Table 5 and 7). The skin surface temperature for the comparative composition in M3 remained over the entire duration of the measurement below at 29.2° C. or below.

The skin surface temperatures of the untreated controls at M2 and M4 remained constant and laid around the average of the baseline skin surface temperature over the entire measurement period (FIGS. 5, 8 and 9, Tables 5 and 7 and). The temperature difference between the skin surface at and around M1 and the skin surface of untreated skin, particularly at M2 and M4, and comparative composition at M3 was palpable with the fingers.

It can be seen from all thermal views in FIG. 9, that the skin surface temperature shown for M2 to M4 was similar for the entire forearm. Furthermore, on the thermal views at T(tc)₄ to T(tc)₈ it can be seen that the areal at and around M1 was heated.

A reddening was observed for both, for the test composition in M1 and the comparative composition in M3, which was still present at T(tc)₉. The reddening in M3 was presumably due to an allergic reaction. The untreated control site on the left forearm (M4) and on the right forearm (M2) were unaffected, showing that the treatment with the Kytta® heat balm was a locally limited phenomenon.

The subject did not feel any sensations of pain during the entire measurement, neither during the rise in temperature, during the application of the test and control compositions nor in respect of the reddening of the skin, particularly not on and around measurement area M1.

TABLE 7
Skin surface temperatures M1 to M4 at T(tc)ₚᵣₑ,
T(tc)_{DAC} and T(tc)₁ to T(tc)₉ in accordance with FIGS. 8 and 9

| | T(tc)ₚᵣₑ | T(tc)_{DAC} | T(tc)₁ | T(tc)₂ | T(tc)₃ | T(tc)₄ | T(tc)₅ | T(tc)₆ | T(tc)₇ | T(tc)₈ | T(tc)₉ |
|---|---|---|---|---|---|---|---|---|---|---|---|
| | [° C.] | [° C.] | [C] | [° C.] | [° C.] | [° C.] | [° C.] | [C] | [° C.] | [C] | [° C.] |
| M1 (test treatment) | 30.1 | 30.1 | 29.2 | 28.0 | 27.9 | 29.4 | 31.4 | 32.6 | 32.4 | 29.7 | 29.0 |
| M2 (untreated control) | 29.4 | 29.4 | 29.4 | 29.3 | 29.4 | 29.3 | 29.1 | 28.7 | 28.1 | 27.6 | 27.6 |
| M3 (comparative treatment) | 29.2 | 28.1 | 27.9 | 27.4 | 27.5 | 28.0 | 28.5 | 28.9 | 28.0 | 27.3 | 27.2 |
| M4 (untreated control) | 30.0 | 30.1 | 30.0 | 29.8 | 29.8 | 29.7 | 29.6 | 29.2 | 28.7 | 28.2 | 28.1 |

| Point in time | Description |
|---|---|
| T(tc)ₚᵣₑ | initial state before the application of any of the test or comparative compositions |
| T(tc)_{DAC} | 1 sec (0.02 min) after finishing the application of the comparative composition on M3. |
| T(tc)₁ | 1 sec (0.02 min) after finishing the application of the test composition on M1; |
| | 80 sec after measurement start. |
| T(tc)₂ | 210 sec (3.5 min) after finishing the application of the test composition on M1; |
| | 270 sec after measurement start; temperature low point at M3. |
| T(tc)₃ | 270 sec (4.5 min) after finishing the application of the test composition on M1; |
| | 358 sec after measurement start; temperature low point at M1. |
| T(tc)₄ | 600 sec (10 min) after finishing the application of the test composition on M1. |
| T(tc)₅ | 900 sec (15 min) after finishing the application of the test composition on M1. |
| T(tc)₆ | 1800 sec (30 min) after finishing the application of the test composition on M1. |
| T(tc)₇ | 3600 sec (60 min) after finishing the application of the test composition on M1. |
| T(tc)₈ | 5400 sec (90 min) after finishing the application of the test composition on M1. |
| T(tc)₉ | 5820 sec (97 min) after finishing the application of the test composition on M1. |

T(tc)ₚᵣₑ, T(tc)_{DAC} and T(tc)₁ to T(tc)₉ correspond to T(tc)ₚᵣₑ, T(tc)_{DAC} and T(tc)₁ to T(tc)₉, respectively, of FIGS. 8 and 9.

### Discussion of Reference Examples 1, 2, 3 and 4

O2C measurement indicates an increased blood volume within the skin and a vasodilation of the capillaries within the skin. Relation between sO₂ rHb rFlow Vel blood volume within the skin capillaries vasodilation of the skin capillaries, and accumulation of PBMCs within the skin

Most of the capillary blood volume is pooled in the postcapillary venous system. Therefore, as already stated above, the O2C measures mainly in this area. With respect to the blood flow direction, the capillaries of the skin are arranged upstream to the postcapillary venous system. Hence, by measuring changes in the blood volume of the downstream postcapillary venous system, direct conclusions for changes in the skin capillaries can be drawn. An increase in the blood volume pooled in the postcapillary venous system results from an increase in the blood volume leaving the skin capillaries and arriving in the postcapillary venous system. The increased blood volume in the postcapillary venous system was indicated by increased values for the rHb and sO₂ when measured with O2C:
The rHb is directly proportional to the amount of erythrocytes, and hence, to the volume of blood present in the postcapillary venous system.
The sO₂ is directly proportional to the oxygen saturation of the erythrocytes in the postcapillary venous system. A higher sO₂ value indicates that there is a greater proportion of erythrocytes present that are still loaded with oxygen when they exit the skin capillaries. Such greater proportion of oxygenated erythrocytes can either:
   (i) be due to a reduced oxygen consumption in the upstream skin capillaries while the total amount of erythrocytes remains the same; or
   (ii) due to the presence of additional erythrocytes so that the total amount of erythrocytes was increased while oxygen consumption in the upstream capillary system remains the same; or due to a combination of items (i) and (ii).

It is pointed out that the subject did not change her activity level over the entire measurement (muscles relaxed, calm position, no movement, unchanged blood pressure). The environmental conditions did not change or vary either (c.f. material and methods' section “O2C—Oxygen to See”, item (B)). Consequently, the oxygen consumption by the subject's skin tissue and, hence, the amount of oxygen extracted from the erythrocytes within the skin capillaries remained unaltered. Therefore, item (ii) is applicable and the increased s02 value can only be explained by a greater number of oxygenated erythrocytes arriving in the postcapillary venous system. Consequently, an increased amount of erythrocytes, and hence an increased blood volume must have passed through the capillaries of the skin.

Therefore, the values rHb and sO₂ are both measures for the blood volume present in the postcapillary venous system. However, they do not yet allow any statement as to why the blood throughput through the upstream skin capillaries was increased finally leading to the increase in the postcapillary blood volume. Again, two causes are possible, (iii) either the upstream skin capillaries show an increased vasodilation thereby allowing to hold and transport a larger blood volume without any increase in the blood flow velocity (iv) or the blood flow was accelerated while the skin capillary dilation was unchanged or a combination of items (iii) and (iv). To distinguish there between, the O2C determines in addition to the rHb and the sO₂ the blood flow parameters rFlow and Vel. By using a heat pad, as can be seen from Reference Example 1, all 4 parameters sO₂, rHb, rFlow and Vel were increased (FIGS. 2A to 2D). However, when using heat crème, only sO₂ and rHb were increased while the flow parameters rFlow and Vel remained unaffected or were even decreased (Reference Example 2, FIGS. 3A to 3D). An increase in the sO₂ and rHb indicates, as stated above under item (ii), an increase in the amount of erythrocytes and hence, an increase in the blood volume pooled in the postcapillary venous system. Since such increase in Reference Example 2 was not caused by an increase in the flow velocity (Vel unchanged or even decreased), such increased blood volume pooling in the postcapillary venous system must be due to item (iii) that is a vasodilation and an increased blood volume present in the skin capillaries.

In summary, in the present experimental setup, the blood volume in the postcapillary venous system (measured via sO₂ and rHb) is directly proportional to the blood volume present within the lumen of the capillaries of the skin. By determining the blood volume within the postcapillary venous system and the blood flow within the skin tissue, the O2C allows to determine whether an increased blood volume is contained within the skin capillaries and therefore a vasodilation of the skin capillaries is present.

Consequently, Reference Examples 1 and 2 demonstrate that:
generating a vasodilation of the capillaries within the skin, but not necessarily increased blood flow parameters rFlow and Vel (c.f. Reference Example 1 versus Reference Example 2), achieves the beneficial effects of the present invention (c.f. e.g. Inventive Examples 1 and 17, which use like Reference Example 2 heat crème).

Furthermore,
generating an increased blood volume, but not necessarily increased blood flow parameters (Reference Example 1 versus Reference Example 2), within the skin achieves the beneficial effects of the present invention (c.f. Reference Example 1, which uses heat crème).

This also in line with the observed redness (Reference Examples 1 to 4). An increased blood volume has an increased amount of erythrocytes (increased rHb and sO2 values) causing the skin to turn red.
Therefore, generating a redness on the skin of the subject provides for the beneficial effects of the present invention (c.f. e.g. Inventive Examples 1 to 7 and 9 to 17).

Furthermore, this also in line with the increased skin surface temperature measured (Reference Examples 3 and 4). In case of a heat crème, the crème as such was not warm, however, it biochemically acts amongst other as vasodilator (c.f. materials and methods' section ‘Application of heat crème’). Blood from inside the body like the liver increasingly flows into the dilated capillaries. Blood from the liver has a high temperature of up to 40° C., but also from other body regions the temperature is close to 37° C. In case of applying the heat pad, it is well established that by applying heat a vasodilation is induced. The increased volume of such high temperature blood present within the dilated skin capillaries conditions an increased temperature of the skin measurable on the skin surface.
Therefore, generating an increased temperature on the skin of the subject provides for the beneficial effects of the present invention (c.f. e.g. Inventive Examples 1 to 7 and 9 to 17).

Consequently, the increase in the blood volume in the postcapillary system measured by O2C (and indicated by increased values for rHb and/or increased sO₂) is amongst other indicative for the generation of:
a vasodilation of the capillaries within the skin of the subject; and/or
an increased temperature on the skin of the subject; and/or
a redness on the skin of the subject,
and vice versa.

Hence, vasodilation, increased blood volume, increased rHb, increased sO₂, increased temperature and skin redness appear mutually dependent and seem to be, at least partially, cause and result of each other at the same time.

Examples on Patients

All patients treated in the following Inventive Examples, Reference Examples and Comparative Examples were selected solely based on their diagnosis without performing a preselection in view of other parameters.

All patients which were treated in the following Inventive Examples reported an onset of beneficial effects within 12 hours to 24 hours after receiving the treatment.

None of the patient treated, neither in the following Inventive Examples, Reference Examples nor Comparative Examples, showed an increased infection rate, increased susceptibility to infections, adverse side effects nor incompatibilities.

All patients, physicians and all further individuals concerned with performing the examples were subject to confidentiality and were required to sign a confidentiality agreement.

### Inventive Example 1 (IE 1)—Patient 1: Rheumatoid Arthritis

1000 IU IFN-γ (High Dose) and 200 IU IL-2 Plus Heat Crème

Patient 1 was at the beginning of the treatment a 46-year-old female, 58 kg, suffering from rheumatoid arthritis in accordance with code M05.80 of the standard ICD-10-GM 2021 for over 20 years (ICD-10 M05.80). She showed swelling of joints and joint pain as indicated in Table 8A. She was not able to walk downstairs forwardly, could not sleep through and was strongly impaired in daily life. The patient described her condition as strongly affected by the disease.

Over the course of the treatment she received a constant basic treatment of 30 mg hydrocortisone per day. This was necessary because the cortisol production by the adrenal glands of the patient was insufficient. The dose, composition and frequency of the hydrocortisone basic treatment remained constant over the entire duration of the treatment.

All injection solutions were prepared as described in the Materials and Methods' section. Furthermore, in preparation for the treatment, 100 μl of IL-2 injection solution (containing 200 IU IL-2) and 100 μl high dose IFN-γ injection solution (containing 1000 IU IFN-γ) were combined in order to prepare 200 μl of a combined injection solution.

The treatment according to the present invention was performed by topically applying on the skin of the upper side of the forearm a heat crème, Kytta® heat crème, as described in the Materials and Methods' section ‘Application of heat crème’. About 50 mg to 800 mg Kytta® heat crème were applied to an area of about 25 cm². After about 1 min to 15 min the skin had turned red and warm and an increase in the skin temperature was palpable. About 1 min to 15 min after the application of the heat crème, independently whether the skin had already turned red and warm or not, the 200 μl of the combined injection solution (containing 1000 IU IFN-γ and 200 IU IL-2) or 100 μl of high dose IFN-γ/IL-2 injection solution (also containing 1000 IU IFN-γ and 200 IU IL-2) were injected within the skin of the red and warmed skin area. The exact treatment regimen is stated in Table 8A. Within the first 22 weeks of the treatment the injection was placed subcutaneously, then the injection depth was lowered to perform an intradermal injection. In both cases a characteristic skin wheal was raised, wherein in the latter case it was more pronounced. The depth of injection was at the beginning about 2 mm to 5 mm and then lowered to about 1 mm to 2.5 mm underneath the surface of the skin. The treatment regimen was performed as indicated in Table 8A.

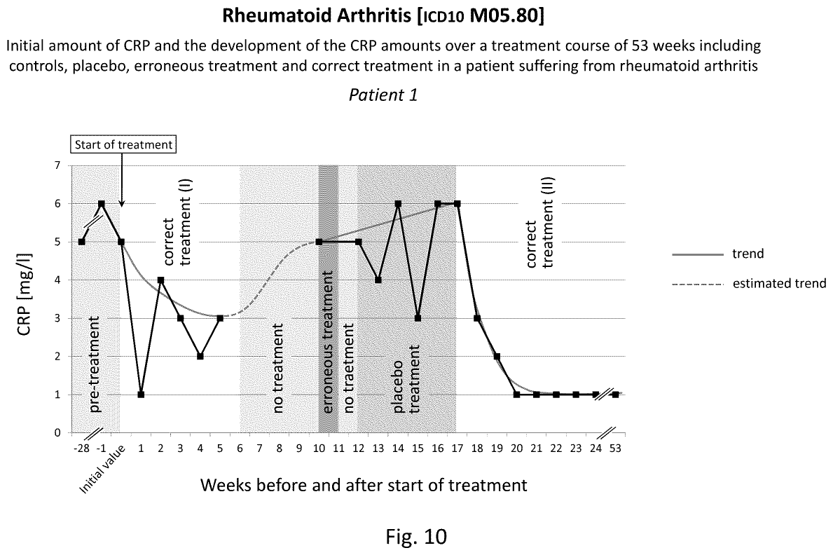

According to the patient's statement, over about the first 1.5 weeks of treatment pain and fatigue were significantly reduced. Within the further course of the treatment the swelling in the joints and the tenderness/pain of the joints were strongly reduced. The patient was able to walk downstairs forwardly, could sleep through and was less impaired in daily life. The CRP decreased remarkably. Details and results are indicated in Table 8A and FIGS. 10 to 13. The patient then described her condition as good. It is pointed out that in week 51 the CRP was temporarily increased to 5 mg/l. This could be explained due the fact that the patient suffered from a bacterial infection and furthermore had not been treated the week before.

In weeks 6 to 9 and 11 after the start of treatment, no treatments were performed. It can be seen from Table 8A and FIG. 10, that after 4 weeks without treatment the CRP returned to 5 (week 10 after start of treatment), that is equal to the initial value at the start of treatment.

It is noted that patient 1 that in patient 1 furthermore an incorrect/erroneous treatment (week 10 after start of treatment) and a double blinded placebo treatment (weeks 12 to 16 after start of treatment) were performed, which are described in Comparative Example 1, Comparative Example 3, Table 8A and FIG. 10.

The patient was treated for 53 weeks until date and no accommodation with respect to the treatment, so no reduction in effect of the treatment was observed. Furthermore, neither adverse side effects nor intolerances nor incompatibilities, also not with the hydrocortisone basic treatment, were observed over the entire duration of the treatment.

As regards the skin temperature when using heat crème, it is pointed out that the skin temperature about 4.5 min after the application of the heat crème was even lower than the initial skin temperature (c.f. Reference Example 4 and FIG. 8 at T(tc)₃,). However, about 15 min after the administration of the IFN-γ and IL-2 the temperature had strongly increased and exceeded clearly the initial skin temperature (FIG. 8 at T(tc)₅). This indicates that an accumulation of PBMCs, vasodilation, increased blood volume, increased sO₂, increased rHb, increased temperature and/or redness is also sufficient to be established after injecting the immunomodulatory substance(s) in order to achieve the beneficial effects.

### Inventive Example 2 (IE 2)—Patient 2: Rheumatoid Arthritis

100 IU IFN-γ (low dose); or 100 IU IFN-γ (low dose) and 200 IU IL-2 plus heat pad Patient 2 was at the beginning of the treatment a 59-year-old female, 62 kg, suffering from rheumatoid arthritis in accordance with code M05.80 of the standard ICD-10-GM 2021 for over 20 years. She showed deformations and swelling of joints and joint pain, particularly, in both hands (all ten proximal interphalangeal and methacarophalangia were affected). The patient was not able to grab properly and unable to open bottles. The patient described her condition as strongly affected by the disease.

Over the course of the treatment, she received a constant basic treatment of 15 mg methotrexate (MTX; folic acid antagonists) per week. However, after the second treatment, she forgot to inject the MTX. Her condition and pain had already improved to such an extent that she was no longer in need of the MTX. Nevertheless, she continued the treatment with MTX but reduced it in week 11 to 12.5 mg/week and further in week 44 to 10 mg/week. Furthermore she did not use it in weeks 43 and 47 after treatment start. At the present time it was the patient's final aim to become free of MTX. Thus the weekly MTX dose will continue to be reduced beyond the duration of this example while the treatment is continued.

All injection solutions were prepared as described in the Materials and Methods' section. Furthermore, in preparation for the treatment, 100 μl of IL-2 injection solution (containing 200 IU IL-2) and 100 μl of low dose IFN-γ injection solution (containing 100 IU IFN-γ) were combined in order to prepare 200 μl of a combined injection solution.

The treatment according to the present invention was performed by topically applying a heat pad on the upper side of the forearm as described in the Materials and Methods' section ‘Application of a heat pad’. The heat pad was applied for about 2 min. Thereafter the skin had turned red and warm and an increase in the skin temperature was palpable. About 1 min after removing the heat pad, 100 μl of low dose IFN-γ injection solution (containing 100 IU IFN-γ), 200 μl of the combined injection solution (containing 100 IU IFN-γ and 200 IU IL-2) or 100 μl of low dose IFN-γ/IL-2 injection solution (also containing 100 IU IFN-γ and 200 IU IL-2) were injected to administer the amounts according to the regimen as stated in Table 8A. In particularly, from week 12, solely 100 IU IFN-γ (without IL-2) were injected within the skin of the red and warmed skin area.

In the first three treatments the injection (initial and weeks 1 and 2 after start of treatment) was placed subcutaneously. In the following treatments the injection depth was lowered to perform the injection intradermally. In both cases a characteristic skin wheal was raised, wherein in the latter case it was more pronounced. The depth of injection was at the beginning about 2 mm to 5 mm and then lowered to about 1 mm to 2.5 mm underneath the surface of the skin.

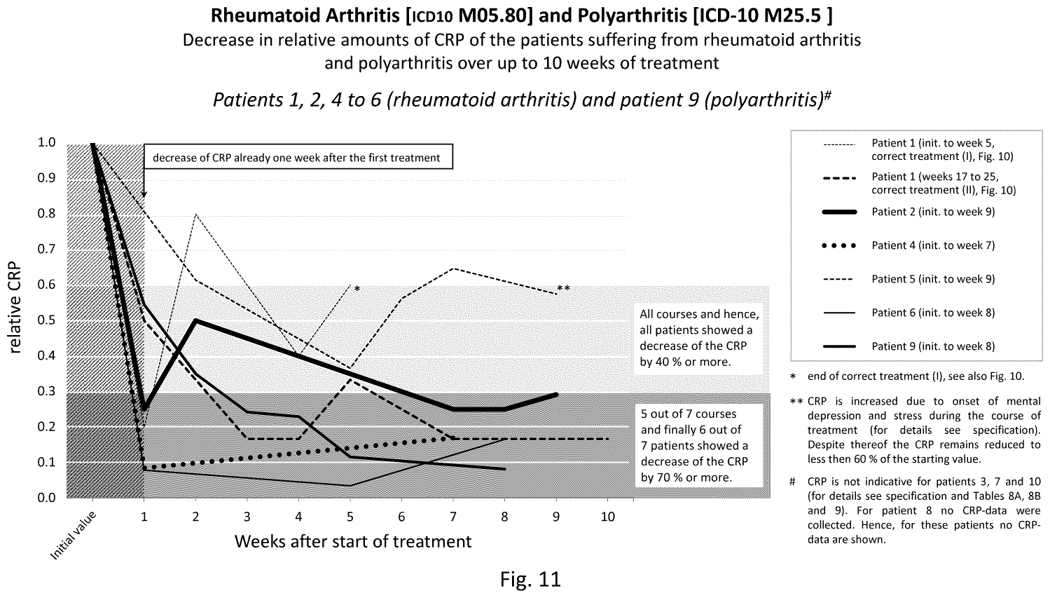

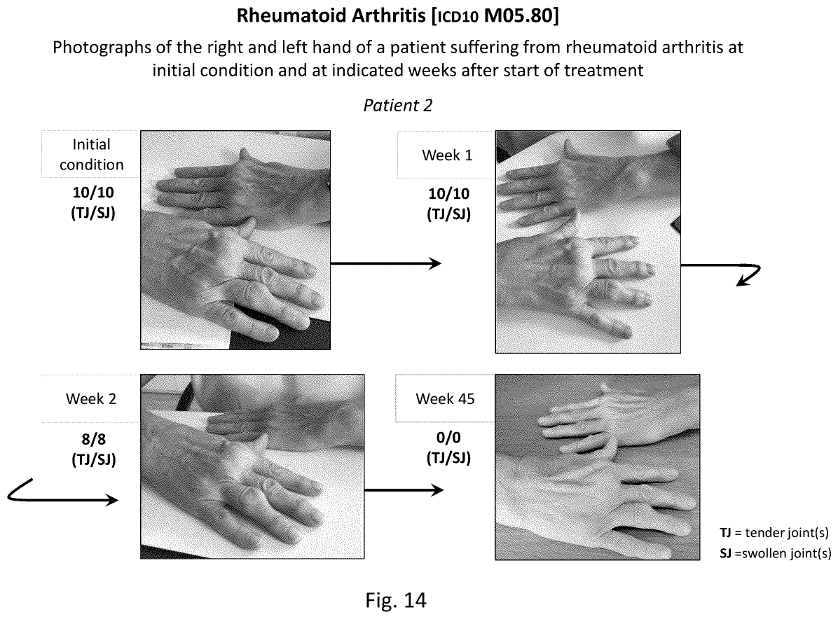

As already stated above, after the second treatment the patient's condition had improved that much that she forgot to inject the MTX. Furthermore, the patient reported that the fatigue was reduced. The CRP decreased already remarkably within the first two weeks and did not rise even after 4 weeks without treatment (weeks 3 to 6) and finally levelled off at below 6 mg/l (Table 8A and FIGS. 11 to 13). Furthermore, two weeks after start of treatment the number of tender (TJ) and swollen (SJ) joints was already reduced from initially 10/10 (TJ/SJ) to 8/8 (TJ/SJ) and the HAQ had become zero (Table 8A and FIG. 14). After 10 weeks of treatment including interruptions (no treatment in weeks 3 to 6 and 10) the initial number of tender and swollen joints of 10/10 (TJ/SJ) in both hands were reduced to zero 0/0 (TJ/SJ) and this condition was maintained (Table 8A and FIG. 14).

The treatment was performed for 50 weeks without relapse. The CRP stayed at below 0.6 mg/l, the HAQ at zero and the number of tender and swollen joints became also zero, except for week 42 (Table 8A and FIGS. 11 to 14). The patient was treated for 50 weeks and no accommodation with respect to the treatment, so no reduction in effect of the treatment was observed. Neither adverse side effects nor intolerances nor incompatibilities, also not with the MTX basic treatment, were observed over the entire duration of the treatment. The treatment even added a beneficial effect on top of the MTX basic treatment and allowed reducing and gradually even substituting the MTX while maintaining the improved health condition.

### Inventive Example 3 (IE 3)—Patient 3: Rheumatoid Arthritis

100 IU IFN-γ (Low Dose); or 100 IU IFN-γ (Low Dose) and 200 IU IL-2 or without IL-2 Plus Heat Pad

Patient 3 was at the beginning of the treatment an 83-year-old female, 78 kg, suffering from rheumatoid arthritis in accordance with code M05.80 of the standard ICD-10-GM 2021 for over 45 years. It is noted that in patient 3 the first treatment was an erroneous treatment which is described in Comparative Example 2 (c.f. Table 8A).

Patient 3 showed deformations and swollen and tender joints in both shoulders, knees and hands including all ten proximal interphalangeal and methacarophalangia. The patient was not able to walk downstairs forwardly, perform gardening and was strongly impaired in housekeeping. The patient received a basic treatment with pain killers, however, the patient reported not to be free of pain despite the pain killers. The patient described her condition as strongly affected by the disease.

Over the course of the treatment the basic treatment with pain killers was continued. The dose, composition and frequency of the pain killer basic treatment remained constant over the duration of the treatment.

All injection solutions were prepared as described in the Materials and Methods' section. Furthermore, in preparation for the treatment, 100 μl of IL-2 injection solution (containing 200 IU IL-2) and 100 μl low dose IFN-γ injection solution (containing 100 IU IFN-γ) were combined in order to prepare 200 μl of a combined injection solution.

The treatment according to the present invention was performed by topically applying a heat pad on the upper side of the forearm as described in the Materials and Methods' section ‘Application of a heat pad’. The heat pad was applied for about 1 min to 2 min. Thereafter, the skin had turned red and warm and an increase in the skin temperature was palpable. About 1 min to 2 min after removing the heat pad, 100 μl of low dose IFN-γ injection solution (containing 100 IU IFN-γ), 200 μl of the combined injection solution (containing 100 IU IFN-γ and 200 IU IL-2) or 100 μl of low dose IFN-γ/IL-2 injection solution (also containing 100 IU IFN-γ and 200 IU IL-2) were injected to administer the amounts according to the regimen as stated in Table 8A. Hence, in weeks 1 to 6 after start of treatment 100 IU IFN-γ (without IL-2) were injected, in weeks 8 to 10 after start of treatment 100 IU IFN-γ and 200 IU IL-2 were co-injected, each within the skin of the red and warmed skin area. In the first three treatments the injection was placed subcutaneously, then the injection depth was lowered to perform the injection intradermally. In both cases a characteristic skin wheal was raised, wherein in the latter case it was more pronounced. The depth of injection was at the beginning about 2 mm to 5 mm and was then lowered to about 1 mm to 2.5 mm underneath the surface of the skin.

As can be seen from Table 8A, the low concentration of 100 IU IFN-γ without IL-2 (weeks 1 to 6 after start of treatment) significantly improved the HAQ from initially 1.625 to 1 (week 8) (a decrease in the HAQ of 0.5 score-points or more is regarded as significant, c.f. Materials and Methods' section ‘HAQ—Health Assessment Questionnaire Functional Disability Index’). The patient reported to be free of pain, apart from lumbago, for about 3 to 4 days after treatment, but not over an entire week. Similarly, fatigue was reduced for about 3 to 4 days after treatment, but also not over an entire week. Furthermore, the low concentration of 100 IU IFN-γ without IL-2 did not have any improving effect on the joint condition. The amount of tender (TJ) and swollen (SJ) joints remained unchanged at 30/30 (TJ/SW), the CRP was already initially quite low (1.6 mg/l) and could not be reduced any further.

However, when the low amount of 100 IU IFN-γ was combined with 200 IU IL-2 by injecting the combined injection solution or the low dose IFN-γ/IL-2 injection solution, the patient reported, that the pain free condition, apart from lumbago, and the reduced fatigue were prolonged for the entire week and lasted until the next treatment. Moreover, the amount of tender and swollen joints could be reduced from 30/30 to 20/20 (TJ/SJ) in week 10 after start of treatment while the HAQ remained significantly improved at 1.125 (weeks 10 and 11).

Hence, IL-2 synergistically improved the effect of IFN-γ (c.f. also Comparative Examples 1 and 2). Neither adverse side effects nor intolerances nor incompatibilities, also not with the basic treatment of pain killers, were observed over the entire duration of the treatment. The treatment even added a beneficial effect on top of the basic treatment of pain killers by achieving a further reduction of pain.

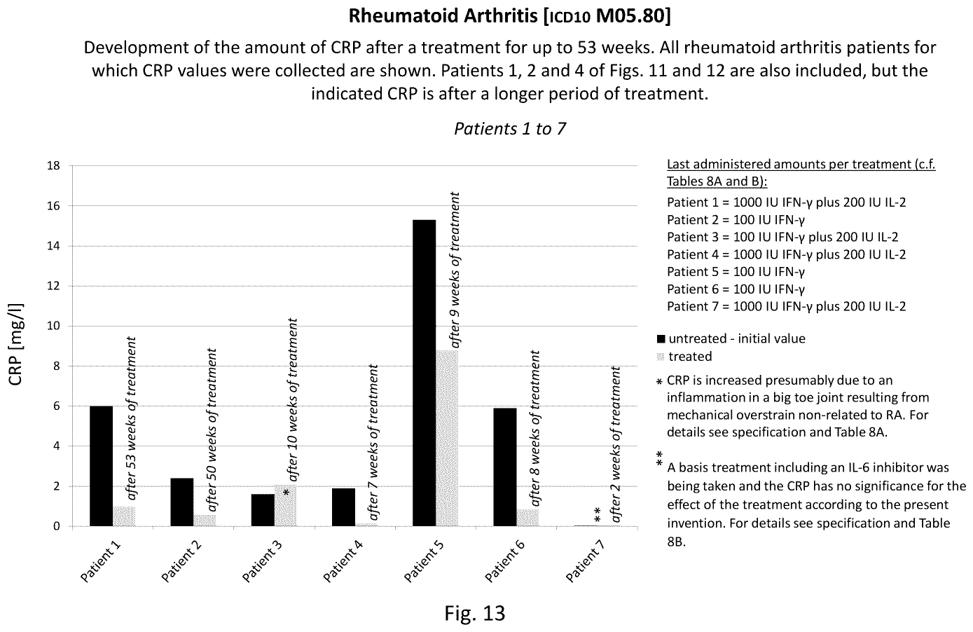

It should be noted that in week 8 the CRP had interims strongly increased to 7.1 (Table 8A and FIG. 13). This presumably due to an inflammation in a big toe joint resulting from mechanical overstrain non-related to rheumatoid arthritis. Consequently, the following decrease in the CRP (weeks 9 and 10 after treatment start) may not be related to the treatment but rather to a healing of the overstrain. As already stated above, the CRP is a highly sensitive marker and already any tiny focus of inflammation may already cause a rise of the CRP.

### Inventive Example 4 (IE 4)—Patient 4: Rheumatoid Arthritis

1000 IU IFN-γ (High Dose) and 200 IU IL-2 Plus Heat Creme or Heat Pad

Patient 4 was at the beginning of the treatment a 49-year-old female, 55 kg, suffering from rheumatoid arthritis in accordance with code M05.80 of the standard ICD-10-GM 2021 for about 35 years. Her rheumatoid condition was already very advanced.

She showed strong deformations of her joints and had received already several joint replacements (>10, exactly 29). Furthermore, she showed swelling of joints and joint pain (tender joints) as indicated in Table 8A. The patient described her condition was strongly affected by the disease.

Over the course of the treatment, she received a constant basic treatment of a combination of several medicaments. The dose, composition and frequency of the basic treatment remained constant over the duration of the treatment.

All injection solutions were prepared as described in the Materials and Methods' section. Furthermore, in preparation for the treatment, 100 μl of IL-2 injection solution (containing 200 IU IL-2) and 100 μl of high dose IFN-γ injection solution (containing 1000 IU IFN-γ) were combined in order to prepare a combined injection solution.

The treatment regimen was conducted as indicated in Table 8A.

In the first three treatments, the treatment was performed by applying a heat crème, Kytta® heat crème, topically on the skin of the upper side of the forearm as described in the Materials and Methods' section ‘Application of heat crème’. In total about 50 mg to 800 mg Kytta® heat crème were applied to an area of about 50 cm². After about 6 min, 200 μl of the combined injection solution (containing 1000 IU IFN-γ and 200 IU IL-2) were injected within the skin of the area the heat crème had been applied to.

In weeks 5, 7 and 9 the treatment was performed by topically applying a heat pad on the upper side of the forearm as described in the Materials and Methods' section ‘Application of a heat pad’. The heat pad was applied for about 2 min to 5 min. Thereafter, the skin had turned red and warm and an increase in the skin temperature was palpable. About 1 min to 2 min after removing the heat pad, the combined injection solution was injected within the skin of the red and warmed skin area.

In the first three treatments (initial and weeks 1 and 2 after start of treatment) the injection was placed subcutaneously, then (weeks 5, 7 and 9 after start of treatment) the injection depth was lowered to perform the injection intradermally. In both cases a characteristic skin wheal was raised, wherein in the latter case it was more pronounced. The depth of injection was at the beginning about 2 mm to 5 mm and was then lowered to about 1 mm to 2.5 mm underneath the surface of the skin.

As can be seen from Table 8A and FIGS. 11 to 13, the CRP decreased already after the first treatment from initially 1.89 mg/l to a remarkably low value of 0.16 mg/l. Furthermore, the tender (TJ) and swollen joints (SJ) were strongly reduced from initially 14/11 (TJ/SJ) to 3/2 (TJ/SJ). The patient reported that the treatment was good for her and made her everyday life easier. Neither adverse side effects nor intolerances nor incompatibilities, also not with the basic treatment, were observed over the entire duration of the treatment. However, the pain was not reduced over the entire period of treatment, which was to be expected for patient 4. The degeneration of her joints was already very advanced. The pain resulted from arthrosis and exisiting joint degeneration and was not primarily due to joint inflammation. The immunomodulatory method according to the present invention was not expected to improve or restore destructed and deformed joints. The treatment aims, amoungst others, at the control and prevention of joint inflammation. The example shows that the heat crème can be exchanged with a heat pad. Hence, the patient preferred the treatment using a heat pad, which is in most cases easier to perform. It takes time for the heat crème to be absorbed. By then the skin is greasy, which can be a hindrance. In addition, Kytta heat crème may cause adverse side effects, such as allergic reactions and burning skin. All this not the case when using a heat pad.

As already described in Inventive Example 1, when using heat crème, the skin temperature after application of the heat crème first decreases before increasing and then exceeding the initial skin temperature value (c.f. Reference Example 4 and FIG. 8 at T(tc)₃,). Hence, also Inventive Example 4 indicates that an accumulation of PBMCs, vasodilation, increased blood volume, increased sO₂, increased rHb, increased temperature and/or redness is also sufficient to be established after injecting the immunomodulatory substance(s) in order to achieve the beneficial effects.

### Inventive Example 5 (IE 5)—Patient 5: Rheumatoid Arthritis

100 IU IFN-γ (Low Dose) Plus Heat Pad (No IL-2 Administered)

Patient 5 was at the beginning of the treatment a 57-year-old female, 75 kg, suffering from rheumatoid arthritis in accordance with code M05.80 of the standard ICD-10-GM 2021. She showed swelling of joints and joint pain as indicated in Table 8B. Both wrists, knees and feet were affected. She had received joint replacements (total knee prostheses) already 3 and 5 years before start of treatment. The patient was strongly impaired in daily life and described her condition as strongly affected by the disease. The patient refused any other form of treatment with a strong aversion against drugs.

The treatment according to the present invention was performed by topically applying a heat pad on the upper side of the forearm as described in the Materials and Methods' section ‘Application of a heat pad’. The heat pad was applied for about 1 min to 4 min. Thereafter, the skin had turned red and warm and an increase in the skin temperature was palpable. About 15 sec to 2 min after removing the heat pad, 100 μl of low dose IFN-γ injection solution (containing 100 IU IFN-γ) were injected within the skin of the red and warmed skin area. The injection solution was prepared as described in the Materials and Methods' section. The treatment regimen was conducted as indicated in Table 8B.

The injection was placed intradermally. A characteristic and pronounced skin wheal was raised by the injection. The depth of injection was about 1 mm to 2.5 mm underneath the surface of the skin. The treatment regimen was performed as indicated in Table 8B.

One week after the first treatment, the patient described an improvement of her aching feet. After the second treatment the number of tender joints was reduced from initially 22 to 8 (Table 8B). Five weeks after start of treatment, the CRP had remarkably decreased from initially 15.3 mg/l to 5.6 mg/l, the number of tender and swollen joints was reduced to 0/8 (TJ/SJ), the HAQ was improved from initially 1.75 to 1.125 and she (a farmer) reported that the milking of the cows was easier to do.

From the fifth week after the start of treatment, the patient was exposed to severe mental pressure and confronted with personal problems. She made a strongly stressed and depressed impression and the CRP increased from week 6 until week 9 which can be explained by her stressful situation. An elevated CRP is known to show an association with stressful conditions (c.f. also Materials and Methods' section ‘CRP—C-reactive protein’). Nevertheless, the CRP at week 9 after start of treatment was 8.8 mg/l which is still about only 60% of the initial CRP of 15.3 mg/l (Table 8B and FIGS. 11 and 13). Furthermore, despite her stressful life conditions, the HAQ and the number of tender and swollen joints did not deteriorate and remained on the improved level or continued to further improve (Table 8B). The patient finally stopped the treatment for personal reasons due to her renewed strong aversion against any kind of medication and her depressed mental condition.

### Inventive Example 6 (IE 6)—Patient 6: Rheumatoid Arthritis

100 IU IFN-γ (Low Dose) Plus Heat Pad

Patient 6 was at the beginning of the treatment a 71-year-old female, 62 kg, suffering from rheumatoid arthritis in accordance with code M05.80 of the standard ICD-10-GM 2021. She showed swelling of joints and joint pain as indicated in Table 8B. The patient described her condition as strongly affected by the disease.

Over the course of the treatment, she received a basic treatment of 600 mg ibuprofen (non-steroidal anti-inflammatory drug) twice per day, 50 mg tilidin (synthetic opioid) once per day in the evening and 200 mg novalgin (metamizole; analgetic and antipyretic) once per day in the morning.

The treatment according to the present invention was performed by administering topically a heat pad on the upper side of the forearm as described in the Materials and Methods' section ‘Application of a heat pad’. The heat pad was applied for about 1 min to 5 min until the skin had turned red and warm and an increase in the skin temperature was palpable. About 15 sec to 1 min after removing the heat pad, 100 μl of low dose IFN-γ injection solution (containing 100 IU IFN-γ) were injected within the skin of the red and warmed skin area. The injection solution was prepared as described in the Materials and Methods' section. The treatment regimen was performed as indicated in Table 8B. The injection was placed intradermally to intraepidermally. A characteristic and pronounced skin wheal was raised. The depth of injection was about 0.5 mm to 2.5 mm underneath the surface of the skin.

The day after the first treatment, the patient reported about reduced muscular tension and less stiffness in the morning. The second day after the first treatment, the patient was free of pain and did not take pain killers. The positive effect diminished on day 4 after the start of treatment but was restored with the second and consecutive treatments. Thus, the overall fitness was improved and the patient reported to be less impaired in daily life. The CRP decreased remarkably from initially 5.2 mg/l to 0.86 mg/l after 8 weeks of treatment (Table 8B and FIGS. 11 and 13). Neither adverse side effects nor intolerances nor incompatibilities, also not with the basic treatment indicated above, were observed over the entire duration of the treatment. Over the course of the treatment, the patient reduced the pain killers on her own decision, because she became more and more pain free. Hence, the treatment even allowed reducing and gradually substituting the pain killers while maintaining the improved health condition. The patient then described her condition as good.

### Inventive Example 7 (IE 7)—Patient 7: Rheumatoid Arthritis/Synovitis

1000 IU IFN-γ (High Dose) and 200 IU IL-2 Plus Heat Pad Patient 7 was at the beginning of the treatment a 55-year-old female, 48 kg, suffering from rheumatoid arthritis in accordance with code M05.80 of the standard ICD-10-GM 2021 and strong synovitis in accordance with code M65.- of the standard ICD-10-GM 2021. She showed swelling of joints and joint pain as indicated in Table 8B. Both elbows, wrists, hands and feet were strongly affected by the disease and painful. The metacarpi I and II of the right hand were surgically replaced and an arthrodeses were performed on the metacarpi II to V few weeks before treatment start. The joints of the hands showed strong synovitis. The patient described her condition as strongly affected by the disease. Furthermore, both patient's forefeet were pathologically strongly deformed leading to swelling and pain which, however, was non-attributable to her rheumatoid arthritis.

Furthermore, the patient received until a line more than a year before start of treatment several distinct biologics and MTX (methotrexate). Precisely, over the years 2008 to April 2021 the patient received treatments with:
three distict TNF-α-inhibitors, namely:
   adalinumab (Imraldi®, adalinumab is the active agent also contained in Humira®);
   etanercept (Enbrel®); and
   certolizumab pegol (Cimzia®),
three distinct Janus-kinase (JAK)-inhibitors, namely:
   baricitinib (Olumiant®);
   tofacitinib (Xeljanz®); and
   upadacitinib (Rinvoq®),
and furthermore:
   abatacept (Orencia®, selective immunosuppressive inhibiting T-cell activation); and
   methotrexate (MTX).

Due to insufficient effectiveness like for instance in case of abatacept (Orencia®) and/or very severe adverse side effects like the tendency to fibrosis in the lungs with fungal manifestation and aspergilloma in the lungs, sinusitis, pansinusitis, rheumatoid nodules and herpes zoster all indicated treatments with the biologics and MTX needed to be stopped. A treatment regimen with MTX and upadacitinib (Rinvoq®) was tried even a second time, but again severe adverse effects (tendency to fibrosis in the lungs and sinusitis) developed and a discontinuation of the treatment was necessary.

Over the course of the treatment according to the present invention, she received a basic treatment of 162 mg tocilizumab (RoActemra®, IL-6 inhibitor) per week, 4 mg prednisolone (synthetic glucocorticoid) per day and 60 mg raloxifen (non-steroidal selective estrogen receptor modulator against osteoporosis) per day. Furthermore, until 13 days before start of treatment 20 mg hydroxychloroquin sulphate (Quenzyl®; anti-inflammatory drug for autoimmune diseases) per day were taken. The treatment with tocilizumab (RoActemra®, IL-6 inhibitor) was only partially efficient.

The treatment according to the present invention was performed by administering topically a heat pad on the upper side of the forearm as described in the Materials and Methods' section ‘Application of a heat pad’. The heat pad was applied for about 1 min to 5 min until the skin had turned red and warm and an increase in the skin temperature was palpable. About 15 sec to 1 min after removing the heat pad, 100 μl of high dose IFN-γ/IL-2 injection solution (containing 1000 IU IFN-γ and 200 IU IL-2) was injected within the skin of the red and warmed skin area. The injection solution was prepared as described in the Materials and Methods' section. The treatment regimen was performed as indicated in Table 8B. The injection was placed intradermally. A characteristic and pronounced skin wheal was raised. The depth of injection was about 1 mm to 2.5 mm underneath the surface of the skin.

Under treatment, the HAQ sank significantly from 1.75 before treatment to 1.125 within 6 weeks of treatment and further to zero. After 12 weeks of treatment (Table 8B and FIG. 15). Furthermore, a reduction in the amount of tender and swollen joints from initial 5/6 (TJ/SJ) to 0/1 was effected within 12 weeks of treatment (Table 8B, indicated in bold digits). The intermediate rise to 7/8 (TJ/SJ) in week 7 is attributable to a maltreatment in post-operative occupational therapy on the operated right hand that also caused the patient a lot of psychological distress. The amounts of swollen and tender joints indicated in Table 8B in brackets and italic digits resulted from the deformations and are non-related to rheumatoid arthritis. As already stated above, both forefeet are pathologically strongly deformed leading to swelling and pain.

Hence, the treatment according to the present invention provided a beneficial effect on top of the IL-6 inhibitor (and the other substances of the basic treatment) that the IL-6 inhibitor alone could not have achieved. The immunomodulatory method aims, amongst others, at the control of joint inflammation and the prevention or delay of joint degeneration, but it was not expected to improve or restore deformed joints. Since the patient received the IL-6 inhibitor, the CRP was not a decisive marker. IL-6 is known to be necessary for CRP gene induction. The IL-6 inhibitor accounts for the absence of elevated CRP values already before the start of treatment according to the present invention and has no significance for the effect of the treatment according to the present invention as long as a IL-6-inhibitor is being taken.

Furthermore, the patient reduced the pain killers on her own decision during the treatment, because she be became more and more pain free. This demonstrates that the treatment allowed reducing and gradually substituting the pain killers while maintaining the improved health condition. Furthermore, the treatment substituted the Quensyl® (hydroxychloroquin sulphate) while maintaining the improved health condition. Half a year before the start of treatment, the patient already did not take the Quensyl® for about two weeks, but then had to continue taking it, because RoActemra® (tocilizumab) and prednisolone alone, without Quensyl®, was not sufficient to achieve a tolerable state of health/life quality. As stated above, at the start of the treatment, the Quensyl® had been again suspended for already 13 days. The treatment according to the present invention allowed to improve the patient's state of health/life quality in the absence of a Quensyl® basic treatment. Since then and until now, there was no indication to take Quensyl® again. That it was possible to discontinue Quensyl® was particularly gratifying because Quensyl® can cause irreversible eye damage with long-term use. Furthermore, neither any adverse side effects nor intolerances nor incompatibilities, also not with the basic treatment indicated above including tocilizumab (RoActemra®, IL-6 inhibitor), were observed over the entire duration of the treatment.

Hence, it was demonstrated that the treatment according to the present invention allowed to reduce or even substitute the pain killers and hydroxychloroquin sulphate (Quensyl®) while maintaining or even further improving the patient's health condition.

The patient then described her condition as very good as regards the rheumatoid arthritis. She reported that the work in the house became easier for her, that she would enjoy it more and that she is more motivated to do it, she had more desire, verve and energy, a more serene and relaxed state of mind, more vigor in gardening and furthermore sporting activities such as hiking could be done more often and for longer periods of time.

### Inventive Example 8 (IE 8)—Patient 8: Rheumatoid Arthritis

100 IU IFN-γ (low dose) and 200 IU IL-2; 1000 IU IFN-γ (high dose) and 200 IU IL-2 plus Injection of PBMCs Patient 8 was at the beginning of the treatment a 79-year-old female, 75 kg, suffering from rheumatoid arthritis in accordance with code M05.80 of the standard ICD-10-GM 2021 for over 40 years. She showed deformations and swelling of joints and joint pain in both shoulders, knees and hands (all ten proximal interphalangeal and methacarophalangia). She was and is still treated with pain killers. The patient was not able to walk downstairs forwardly, perform gardening or needle work and was strongly impaired in housekeeping. The patient described her condition as strongly affected by the disease.

The dose and composition of the pain killers remained constant over the duration of the treatment with PBMCs.

The injection solutions were prepared as described in the Materials and Methods' section. Furthermore, 100 μl of IL-2 injection solution (containing 200 IU IL-2) were combined with 100 μl of low dose IFN-γ injection solution (containing 100 IU IFN-γ) to 200 μl combined injection solution (a) containing 100 IU IFN-γ and 200 IU IL-2. Alternatively, 100 μl of IL-2 injection solution (containing 200 IU IL-2) and 100 μl of high dose IFN-γ injection solution (containing 1000 IU IFN-γ) were combined to 200 μl combined injection solution (b) containing 1000 IU IFN-γ and 200 IU IL-2.

PBMCs were prepared as described in the Materials and Methods' section ‘Preparation of PBMCs’ and administered every two weeks in a single treatment in a total amount of 9×10⁶ PBMCs (9 million PBMCs), prepared in a volume of 200 μl 0.9 NaCl.

The treatment was performed by injecting 200 μl of the prepared PBMCs (9×10⁶ PBMCs). The PBMCs were injected intradermally to subcutaneously into the skin of a forearm. The depth of injection was about 2 mm to 4 mm underneath the surface of the skin and, following the injection, a characteristic and pronounced skin wheal was raised. About 30 min to 45 min after the injection of the PBMCs either the combined injection solution (a) or the combined injection solution (b) was injected in a vicinity of about 1 cm or less around the injection site of the PBMCs. The depth of the injections was again about 2 mm to 4 mm underneath the surface of the skin and, following the injection, a characteristic and pronounced skin wheal was raised again.

Alternatively, the treatment was performed by conducting a first injection of 100 μl of the prepared PBMCs were injected (4.5×10⁶ PBMCs [4.5 million PBMCs]). The PBMCs were injected intradermally to subcutaneously into the skin of a forearm. The depth of injection was about 2 mm to 4 mm underneath the surface of the skin and, following the injection, a characteristic and pronounced skin wheal was raised. About 30 min to 45 min after this injection of the PBMCs either the combined injection solution (a) or (b) was injected in a vicinity of about 1 cm or less around the injection site of the PBMCs. The depth of injection was also about 2 mm to 4 mm underneath the surface of the skin and a characteristic and, following the injection, again a pronounced skin wheal was raised. After further about 30 min to 45 min a second injection of 100 μl of the prepared PBMCs (4.5×10⁶ PBMCs [4.5 million PBMCs]) was conducted in a vicinity of about 1 cm or less around the first injection site of the PBMCs. The second injection of PBMCs was performed in the same way as the first one.

The patient performed the treatment and the injections as a self-treatment.

According to the patient's statement, after about two days for the combined injection solution (a) and after about one day for the combined injection solution (b) the pain was significantly reduced to zero and the dosage of the pain killers administered was halved.

The swelling in the joints was remarkably reduced, the patient was able to walk downstairs forwardly, perform gardening and needle work and the impairment in housekeeping was strongly reduced. The effects lasted for both combined injection solutions (a) and (b) for about 10 days. The patient then described her condition as good.

Hence, the treatment allowed reducing and gradually substituting the pain killers while maintaining the improved health condition. Furthermore, neither adverse side effects nor intolerances nor incompatibilities, also not with the basic treatment indicated above, were observed over the entire duration of the treatment.

Hence, it was demonstrated that PBMCs impart the beneficial effects.

### Inventive Example 9 (IE 9)—Patient 9: Polyarthritis

100 IU IFN-γ (low dose); or 100 IU IFN-γ (low dose) and 200 IU IL-2 plus heat pad

Patient 9 was at the beginning of the treatment a 77-year-old female, 58 kg, suffering from polyarthritis in accordance with code M25.5 of the standard ICD-10-GM 2021 of unknown origin for over 2 years. She showed swelling of shoulder joints and was not able to lift her arm over her head. The patient described her condition as strongly affected by the disease and as highly painful.

Over the course of the treatment, she received a constant basic treatment with pain killers. The dose, composition and frequency of the pain killer basic treatment remained constant over the duration of the treatment.

The treatment according to the present invention was performed by topically applying a heat pad on the upper side of the forearm as described in the Materials and Methods' section ‘Application of a heat pad’. The heat pad was applied for about 1 min to 2 min until the skin had turned red and warm and an increase in the skin temperature was palpable. About 30 sec to 1 min after removing the heat pad, the injection solutions were administered within the skin of the red and warmed skin area. The treatment regimen was conducted as indicated in Table 9.

For the first 4 treatments 100 μl of low dose IFN-γ injection solution (containing 100 IU IFN-γ) were injected subcutaneously. The depth of injection was about 2 mm to 5 mm.

In weeks 4 to 19 after start of treatment 100 μl of low dose IFN-γ/IL-2 injection solution (containing 100 IU IFN-γ and 200 IU IL-2) were injected intradermally. The depth of injection was about 1 mm to 2.5 mm underneath the surface of the skin.

The injection solutions were prepared as described in the Materials and Methods' section.

After each injection a characteristic skin wheal had raised, wherein for the intradermal injection it was more pronounced.

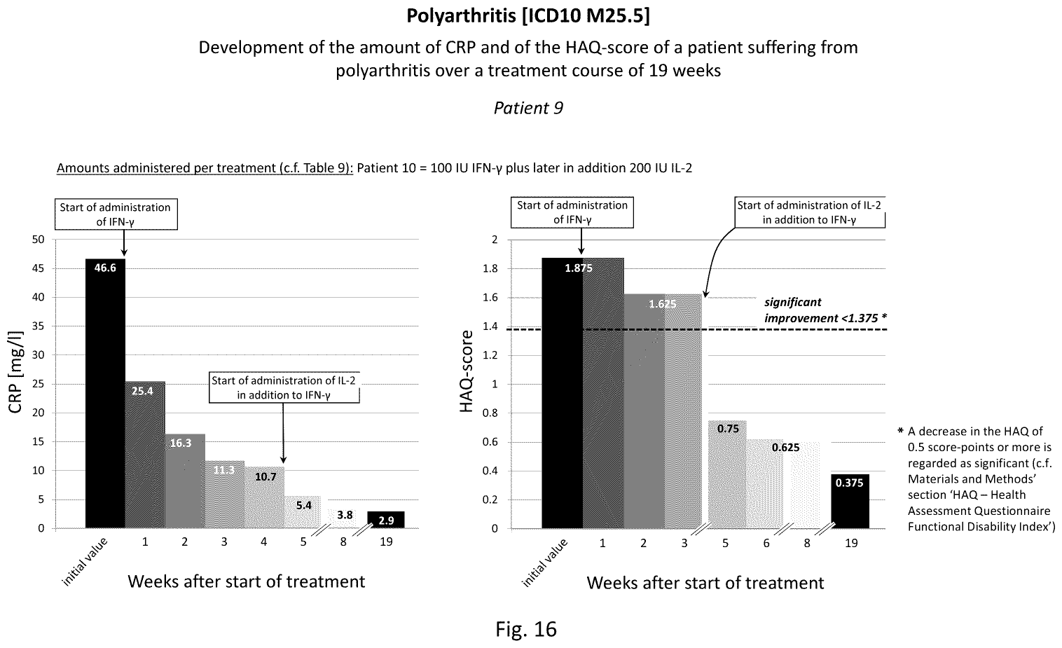

Already after the second treatment (week 2 after start of treatment), the patient was able to lift her hands over her head and the disease condition was strongly improved. The CRP was reduced to almost a third of the initial CRP-amount (week 2, Table 9 and FIGS. 11 and 16). The number of tender joints (TJ) was reduced from 8 to 3 (Table 9). The HAQ decreased significantly within already the first weeks of treatment from initially 1.875 to 0.75 and finally from week 16 to 0.375 (Table 9 and FIG. 16).

This beneficial effects were ongoing for 19 weeks until to date. The final CRP was 2.9 mg/l, which is less than 1/16 (6.25%) of the initial value (FIGS. 11 and 16). The number of tender (TJ) and swollen joints (SJ) was reduced from initially 8/2 (TJ/SJ) to 0/0 (TJ/SJ) and the HAQ decreased significantly to finally 0.375 (Table 9 and FIG. 16). Neither adverse side effects nor intolerances nor incompatibilities, also not with the basic treatment of pain killers, were observed over the entire duration of the treatment. Finally, the patient and physician decided to stop the treatment due to complete remission of symptoms.

### Inventive Example 10 (IE 10): Polyarthritis

100 IU IFN-γ (Low Dose); 100 IU IFN-γ (Low Dose) and 200 IU IL-2; 1000 IU IFN-γ (High Dose) and 200 IU IL-2 Plus Two Times Heat Pad or Electrical Hand Warmer

Patient 10 was at the beginning of the treatment a 58-year-old female, 66 kg, suffering from polyarthritis in accordance with code M25.5 of the standard ICD-10-GM 2021 of unknown origin for over 23 years. The diagnosis of the polyarthritis was done in the year of the start of treatment and before the start of treatment. She showed swelling of finger joints especially the index and the middle finger. The patient described her condition as affected by the disease and as painful.

Over the course of the treatment, she did not receive any basic treatment for polyarthritis.

The treatment according to the present invention was performed by topically applying a heat pad on the upper side of the forearm as described in the Materials and Methods' section ‘Application of a heat pad’. After about 10 weeks of treatment alternatively an electrical hand warmer was used as also described in the Materials and Methods' section ‘Application of a heat pad’.

The heat pad or electrical hand warmer was applied for about 1 min to 2 min until the skin had turned red and warm and an increase in the skin temperature was palpable. About 15 sec to 2 min after removing the heat pad or electrical hand warmer, the injection solutions were administered within the skin of the red and warmed skin area. For the first treatment, 100 μl of low dose IFN-γ injection solution (containing 100 IU IFN-γ) were used for injection. For the 2nd to the 14^{th} treatment 100 μl of low dose IFN-γ/IL-2 injection solution (containing 100 IU IFN-γ and 200 IU IL-2) were used for injection. For all subsequent treatments 100 μl of high dose IFN-γ/IL-2 injection solution (containing 1000 IU IFN-γ and 200 IU IL-2) were used for injection. The injection solutions were prepared as described in the Materials and Methods' section. The injections were placed intraepidermally to intradermally. A characteristic and pronounced skin wheal was raised following the injection. The depth of the injections was about 0.5 mm to 2.5 mm underneath the surface of the skin.

From about the 17^{th} treatment onwards the skin at and around the injection site was reheated one to two times within about 1 hour to hours after the injection was performed by using the electrical hand warmer. At least one reheating was performed, typically about 1.5 hours. The second reheating, if conducted, was usually performed about 3 hours to 4.5 hours after the injection.

After the first treatment, the patient reported to be pain free for 24 hours, but less than a few days.

Hence, the following treatments included IL-2 while the amount of IFN-γ remained unchanged (100 IU IFN-γ). These treatments generally reduced the pain and swelling. Further, the need for sleep was reduced i.e. a shortend sleep duration was sufficient. Moreover, the patient stayed free of pain for a few days but not for an entire week. Hence, similarly to Inventive Example 3, the addition of IL-2 synergistically prolonged the effect of the IFN-γ. After 8 weeks the patient was able to apply crème on her back by herself. When reheating the skin, the patient reported about less tenderness and swelling of the joints during the following days and week (explanation see below).

After increasing the amount of IFN-γ to 1000 IU per week from week 14 after start of treatment, the patient reported to be pain free for about a week. When reheating the skin, the patient reported about even less tenderness and swelling of the joints.

Despite after 22 weeks of treatment, the tenderness and pain of the joints was still slightly visible, the patient reported that the improvement was substantial and the remaining tenderness and pain of the joints was “not comparable to the initial pain and swelling”. The patient indicated only minimal impact on her daily life.

In patient 10 the CRP was not indicative, because the patient was from the beginning and throughout the course of treatment subjected to recurrent major private worries and mental stress both non-related to the treatment. The CRP fluctuated in dependency thereof and it is known that an elevated CRP may be associated with stressful conditions (c.f. also Materials and Methods' section ‘CRP—C-reactive protein’). An HAQ questionnaire was not indicated due the enormous stress and the patient was not able do discriminate between disease and stress related impact on the questions of the HAQ.

Without wishing to be bound to theory, it is believed that by reheating the injection site, more PBMCs can be recruited into the skin. Furthermore, again without whishing to be bound to theory, autoantigen present in low amount in the patient's blood may be accumulated within the skin for a longer period of time. LHs may then be explosed for a longer period of time to the autoantigen and e.g. prolonged or repeated increased temperatures are believed to lead to a better stabilisation of the MCH-autoantigen complex by heat shock protein (chaperone) expression. Thereby, it is believed that e.g. regulatory T-cells are generated more effectively and in larger amounts may be and more regulatory. Consequently, more effector cells like helper T-cells, or a subset thereof, and regulatory T-cells may be generated leading to an amplified beneficial effect. Precisely, as can be seen from e.g. FIGS. 2A, 2B, 3A, 6 and 8, the condition of the skin returns after a certain time (usually about 1 to 1.5 hours) to its initial state, regardless whether a heat pad or a heat crème had been used. Hence, a vasodilation, increased blood volume, increased sO₂, increased rHb, increased temperature and/or redness is no longer present and an accumulation of PBMCs can no longer be generated—the capillaries have constricted again, further PBMCs may not follow into the narrowed capillaries which are not accessible for bulky nucleated cells like PBMCs. However, since the immunomodulatory substance(s) are presumably retained in effective concentrations within the skin tissue for about 6 hours or maybe even longer before they diffuse away diffuse away and/or the dendritic cells presumably conditioned by the immunomodulatory substance(s) are still at site, a repeated recruitment of PBMCs should allow to bring more PBMCs into contact with the immunomodulatory substance(s) and/or the conditioned dendritic cells. Consequently, by repeating the accumulation of PBMCS, vasodilation, increased blood volume, increased sO₂, increased rHb, increased temperature and/or redness more effector cells may be generated and the already beneficial effect may thereby be amplified.

### Inventive Example 11 (IE 11)—Patient 11: Ankylosing Spondylitis (Bechterew's Disease)

1000 IU IFN-γ (High Dose) and 200 IU IL-2; Two Times 100 IU IFN-γ (Low Dose) Plus Heat Creme or Heat Pad

Patient 11 was at the beginning of the treatment a 55-year-old female, 86 kg, suffering from ankylosing spondylitis. She had strong pain, particularly in the back, was suffering from fatigue and was not able to work through for more than 4 days. She was strongly impaired in her work as an accountant and an early retirement was contemplated. She found it difficult to cope with the workday and the psychological workload. Increased physical activity like sports led in the days following the exercise to increased symptoms of Bechterew's disease-related illness and pain. The patient described her condition as strongly affected by the disease.

She reported that a treatment with different biologics (Humira® [adalinumab], Simponi® [golimumab], Imraldi® [adalinumab] and Cimzia® [certolizumab pegol]) had little effect from the outset and within half a year, the biologics lost their effect. The treatement with the biologics ended more than a year before start of the treatment according to the present invention.

Over the course of the treatment, she received a constant basic treatment of a combination of some soporifics and pain killers (oxycodone, cannabis drops, amitriptyline). The dose, composition and frequency of the basic treatment remained constant over the duration of the treatment.

The treatment according to the present invention was conducted in accordance with the regimen as indicated in Table 10. The first four treatments were performed by administering topically on the skin of the upper side of the forearm a heat crème, Kytta® heat crème, as described in the Materials and Methods' section ‘Application of a heat crème’. About 50 mg to 800 mg, usually about 40 mg to 150 mg, Kytta® heat crème were administered to a skin area of about 20 cm² to 70 cm². After about 3 min to 8 min, 100 μl of high dose IFN-γ/IL-2 injection solution (containing 1000 IU IFN-γ and 200 IU IL-2) were injected subcutaneously within the skin of the area the heat crème had been applied to. A characteristic skin wheal was raised by the injection. The depth of injection was about 2 mm to 5 mm underneath the surface of the skin. The injection solution was prepared as described in the Materials and Methods' section.

As already described in Inventive Examples 1 and 4, when using heat crème, the skin temperature after application of the heat crème first decreases before increasing and then exceeding the initial skin temperature value (c.f. Reference Example 4 and FIG. 8 at T(tc)₃,). Hence, also Inventive Example 11 indicates that an accumulation of PBMCs, vasodilation, increased blood volume, increased sO₂, increased rHb, increased temperature and/or redness is also sufficient to be established after injecting IFN-γ and IL-2 in order to achieve the beneficial effects.

In weeks 6, 8 and 10 after the start of treatment, the treatment was performed by topically applying a heat pad on the upper side of the forearm as described in the Materials and Methods' section ‘Application of a heat pad’. The heat pad was applied for about 1 min to 5 min. Thereafter, the skin had turned red and warm and an increase in the skin temperature was palpable. About 30 sec to 2 min after removing the heat pad, 100 μl of high dose IFN-γ/IL-2 injection solution were injected within the skin of the red and warmed skin area. The injection was placed intraepidermally to intradermally. A characteristic and pronounced skin wheal was raised. The depth of injection was keep particularly low. Thus, the depth of injection was about 0.5 mm to 1.5 mm underneath the surface of the skin.

The patient reported about a clear improvement in pain following each treatment. The improvement lasted for about 4 days. Furthermore, the fatigue war reduced and she was able to work two weeks through. Her psychophysical stress resistance was improved and increased physical activity and sports became possible. Moreover, her condition did not deteriorate in the days that followed the increased activity. Even a hula hoop was purchased. However, in some cases at the first day after the treatment her condition improvement was not as much as at the following days or at the day of or the days before the treatment.

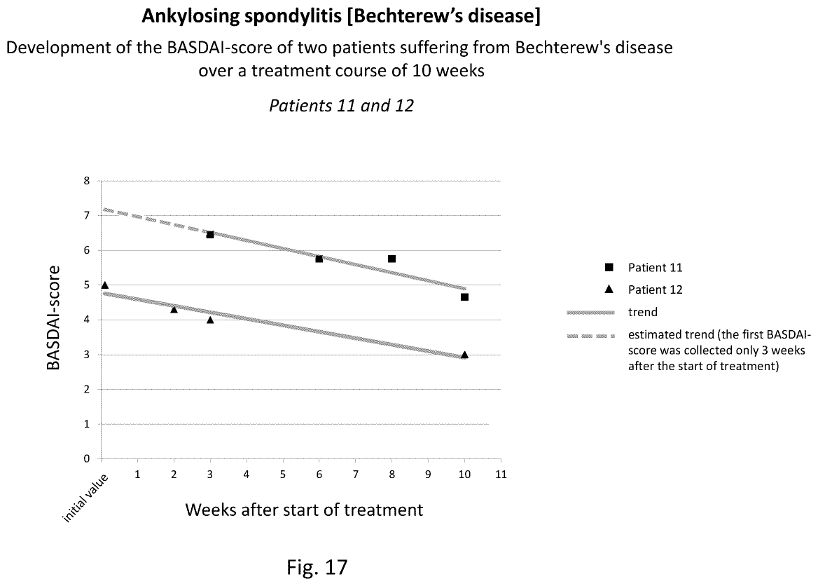

Moreover, the BASDAI-score was determined (Table 10 and FIG. 17). Under treatment in week 10, the BASDAI was reduced to 4.6, which approaches a good control of the disease. A good disease control is indicated by a BASDAI-score of 4 or less (c.f. Materials and Methods' section ‘BASDAI—Bath Ankylosing Spondylitis Disease Activity Index disease activity questionnaire’). Furthermore, regardless whether a heat crème or a heat pad had been used, the number of tender and swollen joints was reduced from initially 7/5 (TJ/SJ) to 3/3 (TJ/SJ) (Table 10).

The example shows that the heat crème can be exchanged with using a heat pad. The treatment using a heat pad is easier and was preferred by the patient. The reason is, that it takes time for the heat crème to be absorbed. By then the skin is greasy, which can be a hindrance. In addition, Kytta heat crème caused a burning skin. All this not the case when using a heat pad. After finishing the trail, the patient insistently requested that the treatment be continued. Hence, a follow-up treatment was carried out as follows:
1) Since some of the effects, particularly the pain relief, lasted under the trail only for about 4 days after the last treatment, this suggested that the weekly frequency of the treatment might be too low and treatment frequency should be increased. Hence, the treatment was continued with two treatments per week (alternating every 3 or 4 days) for about further 15 weeks. Again, in some cases at the first day after the treatment her condition improvement was not as much as at the following days or at the day of or the days before the treatment.
2) Therefore, the frequency of the treatment was reduced to one treatment per week for about another 20 weeks. However, instead of one injection of 100 μl of high dose IFN-γ/IL-2 injection solution (containing 1000 IU IFN-γ and 200 IU IL-2), two injections of 100 μl each of low dose IFN-γ injection solution (containing each 100 IU IFN-γ) were performed. The injection were performed intraepidermally to intradermally in the same manner as stated above. Hence, IL-2 was no longer administered and the total amount of IFN-γ administered was reduced to one fifth. The two injections were placed in a distance of about 1.5 cm to 4 cm to each other and both within the skin of the same area heated beforehand with a head pad as described above. The beneficial effects lasted for 6 to 7 days after treatment. Moreover, the patient reported that the improvement on the day after the treatment was as good and comparable with all other days. According to her observations, this was particularly the case when the injection was placed particularly shallowly, i.e. at a particularly shallow depth underneath the skin surface.

It is pointed out that the follow-up treatment of about 35 weeks in total was carried out by the patient on her own without a doctor. Hence, no CRP values were collected. The overall health condition of the patient remained improved. The patient was more active and her physical and mental resilience were very good. Despite thereof the BASDAI-scores fluctuated, but the values never exceeded the initial level of 6 (maximum score during the follow-up treatment was 5.97 (once), the minimum score was 2.1 (once)) and remained below it on average and median (average BASDAI of 3.86, median BSADAI of 3.96; detailed data are not shown). As stated before, the patient felt good. She therefore tested her limits during the follow-up treatment, increased her activity level and adapted her activity for her improved state of health (longer hikes, longer and more exhausting bicycle tours, longer working days, and even alpine ski tours were undertaken). It seemed that the patient repeatedly overexerted herself. The fluctuations in the BASDAI can be explained that in some cases it seemed difficult for her to distinguish the consequences of the overexertion from the underlying Bechterew's disease. Furthermore, the patient obviously accepted a certain level of discomfort and adjusted her maximum activity level to it (which made up a large part of their quality of life), wherein the activity level and her physical, psychological and occupational resilience were significantly increased compared to before the treatment. In short, with only a moderate improved BASDAI on average and median, significantly more activity and hence significantly more life quality was possible for her.

For personal reasons she interrupted then the treatment for 7 weeks. After about 5 weeks of interruption of the treatment, her condition worsened again and the symptoms typical for her Bechterew's disease reappeared and she decided to resume the treatment soon.

Over the entire duration of the treatment neither adverse side effects nor intolerances nor incompatibilities, also not with the basic treatment indicated above including, were observed over the entire duration of the treatment. There was also no loss of effect of the treatment.

Compared to rheumatoid arthritis, ankylosing spondylitis affects tendons, muscles and connective tissue to a greater extent. Hence, without wishing to be bound to theory, it is believed that by reducing the amount of IFN-γ from 1000 IU to 200 IU and additionally dividing the 200 IU into two injections each containing 100 IU IFN-γ, wherein these injections are placed just below the skin surface (intraepidermally to intradermally), less amounts of IFN-γ diffuse and penetrate into the deeper layers of the skin and the layers below the skin. Hence, the distance of the IFN-γ to muscles and connective tissue and hence, to the autoantigen of ankylosing spondylitis may be increased. Furthermore, by performing two injections with less high amounts of IFN-γ, the IFN-γ is distributed more evenly within the skin. Thereby, a similar or even larger area of the skin (particularly the epidermis and dermis) may be supplied with IFN-γ and can be used as an incubator than when a larger quantity of IFN-γ is injected by one injection. In other words, the amount of IFN-γ may be distributed more targeted in a direction parallel to the skin surface in the epidermis and particularly dermis. Thereby, it is believed that the part of the skin used as an incubator may remain the same or is even enlarged as when a single high IFN-γ dose is injected, and a similar of even larger amount of affected LHs, affected peripheral T_{reg}s and/or affected PBMCs may be generated (prolonged effect of 6 to 7 days after treatment). Moreover, the distance to the antigen is maximized and the exposure of the critical tissues (muscles and connective tissue) to effective amounts of the immunomodulatory substance(s) can at the same time be minimized or prevented. Thereby, it is believed that the patient's immune response in reaction to the treatment can be focused on the generation of anti-inflammatory effectors, particularly regulatory T-cells (c.f. also Inventive Example 12, where similar observations were made). Consequently, this could explain why the health condition was improved to full extent even the day after the treatment.

It is assumed that for this purpose it may be particularly beneficial to divide the total dose of 200 IU IFN-γ to e.g. 50 or more intradermal or intraepidermal injections, which are distributed over a certain skin areal and wherein each injection provides only 4 IU or even much less IFN-γ. This may be for instance accomplished by using a microneedle patch which may be equipped with e.g. 50 or more microneedles for intradermal or intraepidermal IFN-γ administration (patches with 1000 and more microneedles are available). Furthermore, it is assumed that for this purpose it may be particularly beneficial to lower the total IFN-γ dose to 30 IU by at the same time increasing the treatment frequency from 1 to 2 treatments per week to e.g. a daily treatment. It is of course also possible to combine both with each other.

### Inventive Example 12 (IE 12)—Patient 12: Ankylosing Spondylitis (Bechterew's Disease)

100 IU IFN-γ (low dose) plus heat pad

Patient 12 was at the beginning of the treatment a 42-year-old male, 90 kg, suffering from ankylosing spondylitis. He had pain, particularly in the hands and was suffering from morning stiffness. Sports and physical activity were possible only to a very limited extent. Sleeping through the night was difficult. Increased physical activity led to increased symptoms of Bechterew's disease-related illness and pain in the following days. The illness forced him to give up his original profession as a carpenter. The patient described his condition as affected by the disease.

A treatment of the disease with different TNF-α inhibitors (tumor necrosis factor alpha inhibitors) like Enbrel®, Remicade® and Symponi® was accompanied by strong adverse side effects and hence, not successful in this patient. The treatment with TNF-α inhibitors was stopped more than one year before the treatment according to the present invention was started.

The treatment was performed by administering topically a heat pad on the upper side of the forearm as described in the Materials and Methods' section ‘Application of a heat pad’. The heat pad was applied for 1 min to 5 min. Thereafter, the skin had turned red and warm and an increase in the skin temperature was palpable. About 10 sec to 2 min after removing the heat pad, 100 μl of low dose IFN-γ injection solution (containing 100 IU IFN-γ) were injected within the skin of the red and warmed skin area. The treatment regimen was performed as indicated in Table 10. The injection solution was prepared as described in the Materials and Methods' section.

The patient reported about a clear improvement in pain that lasted after the treatment for about 4 days. After four weeks, the morning stiffness was completely absent and did not reuccure since. Sleep became more restful and the patient reported that he is doing very well. Wood chopping and lumbering as well as Nordic walking over distances of 6 km or more became possible and his condition did not deteriorate in the days following the increased physical body activity. Hence, the patient did not suffer a relapse for his increased activity afterwards and his health condition remained unchanged on the improved level. Neither of these activities were possible or possible only with difficulties before start of treatment.

Furthermore, the BASDAI was determined. Three weeks after start of treatment, the BASDAI had reduced to 4 and in week 10 after start of treatment to 3 (Table 10 and FIG. 17). This implies a good control of the disease as indicated by a BASDAI of 4 or less (c.f. Materials and Methods' section ‘BASDAI—Bath Ankylosing Spondylitis Disease Activity Index disease activity questionnaire’). Neither adverse side effects nor intolerances were observed over the entire duration of the treatment.

The injections were placed intraepidermally to intradermally apart from the fourth injection which was placed deeper. In each case a characteristic skin wheal was raised. The patient reported that the beneficial effect was more evident when the substances were injected into a lower depth underneath the skin surface. If the injection was placed deeper, the beneficial effects stated above were achieved, however, an unpleasant neck stiffness occurred. He had not noticed such particular kind of neck stiffness before in his life and he could not explain it otherwise. Hence, the injection was in the further course of the treatment placed particularly low in a depth of about 0.5 mm to 1.5 mm underneath the surface of the skin. The neck stiffness did never reoccur and the beneficial effects remained or even further improved. Hence, an intraepidermal to intradermal administration and a depth of injection of about 0.5 mm to 1.5 mm underneath the surface of the skin showed to be particularly beneficial.

Compared to rheumatoid arthritis, ankylosing spondylitis affects tendons, muscles and connective tissue to a greater extent. Hence, without wishing to be bound to theory, it is believed that by placing the injection less deep and using low dose of IFN-γ, the distance to these tissues and hence to the autoantigen of ankylosing spondylitis can be maximized and/or the exposure of these critical tissues to effective amounts of the immunomodulatory substance(s) can also be minimized or prevented. Thereby, it is believed that the patient's immune response in reaction to the treatment can be focused on the generation of anti-inflammatory effectors, particularly regulatory T-cells (c.f. also Inventive Example 11, where similar observations were made).

### Inventive Example 13 (IE 13)—Patient 13: Polymyalgia Rheumatica

100 IU IFN-γ (Low Dose); Two Times 100 IU IFN-γ (Low Dose); 1000 IU IFN-γ (High Dose) and 200 IU IL-2 Plus Heat Pad

Patient 13 was at the beginning of the treatment a 57-year-old female, 78 kg, suffering from polymyalgia rheumatica in accordance with code M35.3 of the standard ICD-10-GM 2021 of unknown origin for over 2 years. She showed swelling and tenderness of finger and foot joints. The patient described her condition as strongly affected by the disease and as highly painful.

Over the course of the treatment, she received a treatment with pain killers if needed (diclofenac natrium, 7.5 mg) which was reduced gradually by the patient during the treatment course (final dose was a maximum of 7.5 mg per day). Additionally, the patient received once per day each 25 mg hydrochlorothiazide (thiazide diuretic), 50 mg allopurinol (used to treat elevated uric acid levels), 5 mg bisoprolol hemifumarat (beta-blocker for treating increased blood pressure) and 160 mg valsartan (AT₁-antagonist for treating increased blood pressure).

The treatment according to the present invention was performed by topically applying a heat pad on the upper side of the forearm as described in the Materials and Methods' section ‘Application of a heat pad’. The heat pad was applied for about 1 min to 2 min. The skin turned red and became warm and an increase in the skin temperature was palpable. About 30 sec to 1 min after removing the heat pad the injections were performed intradermally within the skin of the red and warmed skin area. The depth of the injection was about 1 mm to 2.5 mm underneath the surface of the skin and a characteristic and pronounced skin wheal was raised by the injection. The treatment regimen is indicated in Table 11. The injections were performed as follows:
For the first treatment, 100 μl of low dose IFN-γ injection solution (containing 100 IU IFN-γ) were used for injection.
For the second treatment (week 1 after start of treatment) two times 100 μl of low dose IFN-γ injection solution (containing each 100 IU IFN-γ) were injected in a distance of about 1 cm within the red and warmed skin.
For the third treatment (two weeks and tree days after start of treatment (week 2[3] after start of treatment)) 100 μl of low dose IFN-γ/IL-2 injection solution (containing 100 IU IFN-γ and 200 IU IL-2) were injected.
For all of the following treatments (3[3] to 9 weeks after start of treatment), 100 μl of high dose IFN-γ/IL-2 injection solution (containing 1000 IU IFN-γ and 200 IU IL-2) were used for injection.

The injection solutions were prepared as described in the Materials and Methods' section.

After the first treatment (week 1 after start of treatment), the patient reported about a decrease of the pain in the first phalanges of the fingers that lasted for one day. After the second treatment (2[3] weeks after start of treatment) the pain in the small finger joints was further reduced. The amount of tender (TJ) and swollen joints (SJ) was reduced from 35/25 (TJ/SJ) (initial value) and 52/52 (TJ/SJ) (week 1 after treatment under dosage finding), respectively, to 26/0 (TJ/SJ) in week 2[3] and 6/0 (TJ/SJ) in week 6[3] after start of treatment. Since the benefit of the treatment did not last longer than 4 days, the treatment was in the following performed twice per week. After the sixth treatment performed in week 5 after start of treatment, the patient reported to be nearly pain-free for 2 days. Since the patient was fully employed, working mainly with her hands in a standing position, a reduction in CRP was hardly expected. Especially, since the workload increased over the course of the treatment and the treatment was not performed in the weeks 5[3] and 6 after start of treatment (due to excess work load and psychical stress, her dog died), this resulted in a relapse. In week 6[3] after start of treatment the CRP had increased to 8.8 mg/l. With resumption of treatment, the CRP could be reduced within two weeks to 6.5 mg/l (week 9 after start of treatment). Furthermore, as can be seen from Table 11, the HAQ was reduced from initially 1.625 to 1.135 (week 9 after start of treatment). The patient then described her condition as good. Neither adverse side effects nor intolerances nor incompatibilities, also not with the basic treatment detailed above, were observed over the entire duration of the treatment.

### Inventive Example 14 (IE 14)—Patient 14: Polymyalgia Rheumatica

100 IU IFN-γ (Low Dose); 1000 IU IFN-γ (High Dose) and 200 IU IL2 Plus Heat Pad

Patient 14 was at the beginning of the treatment a 66-year-old male, 90 kg, suffering from polymyalgia rheumatica in accordance with code M35.3 of the standard ICD-10-GM 2021 of unknown origin for 7 month. He showed swelling and tenderness of all 30 finger joints, and tenderness of both wrists and shoulders. The patient described his condition as affected by the disease and as highly painful. A prompt start of treatment with methotrexate (MTX; folic acid antagonists, disease-modifying antirheumatic drug) was planned. With onset of the disease, the patient was treated with 70 mg prednisolone (synthetic glucocorticoid) which was gradually reduced to 8 mg, but needed to be increased again to 10 mg without complete remission of symptoms.

Over the course of the treatment according to the present invention, he received a basic treatment with binoprolol (beta-blocker for treating increased blood pressure) (3.75 mg) and acetylsalicylic acid (100 mg). With beginning of the treatment according to the present invention, the dose was at 10 mg prednisolone daily. During the treatment according to the present invention, the prednisolone dose was further decreased to 7 mg per day.

The treatment according to the present invention was performed by topically applying a heat pad on the upper side of the forearm as described in the Materials and Methods' section ‘Application of a heat pad’. The heat pad was applied for about 30 sec to 2 min. Following the heat pad application, the skin had turned red and warm and an increase in the skin temperature was palpable. About 1 min after removing the heat pad, 100 μl of low dose IFN-γ injection solution (containing 100 IU IFN-γ) were intradermally injected (first treatment), or 100 μl of high dose IFN-γ/IL-2 injection solution (containing 1000 IU IFN-γ and 200 IU IL-2) were intradermally injected (week 2 to 6 after start of treatment) within the skin of the red and warmed skin area. The depth of the injections was about 1 mm to 2.5 mm underneath the surface of the skin and a characteristic and pronounced skin wheal was raised. The injection solutions were prepared as described in the Materials and Methods' section. The treatment regimen was performed as indicated in Table 11.

After the first treatment (week 1 after start of treatment), the patient reported about a decrease of the pain in the first phalanges of the fingers. After the second treatment (week 3 after start of treatment) the pain in the small finger joints was further reduced. Since the benefit of the treatment did not last longer than for 4 days, the treatment was from week 3 after start of treatment and in the following weeks performed twice per week (alternating every 3 or 4 days). The patient reduced the prednisolone dose in week 2 (before the 2nd treatment) to 9 mg/day, and in week 5 (before the 6^{th} treatment) to 8 mg/day.

During the treatment of 5 weeks and 3 days (Table 11, week 5[3]), the HAQ remained about unchanged, while the activity level and personal quality of life improved according to the patient's statement. The tender and swollen joints reduced from 34/30 (TJ/SJ) to 15/15 (TJ/SJ). The CRP reduced from 2.4 mg/l to 1.8 mg/l (with an interim maximum of 3.4 mg/l and an interim minimum of 1.1 mg/l, Table 11). The patient then described his condition as good. Neither adverse side effects nor intolerances nor incompatibilities, also not with the basic treatment detailed above, were observed over the entire duration of the treatment. The planned treatment with MTX was discarded and was also no longer indicative. Hence, the treatment according to the present invention allowed to substitute the planned treatment with MTX. Furthermore, the treatment allowed reducing and gradually substituting the glucocorticoid while maintaining the improved health condition.

### Inventive Example 15 (IE 15)—Patient 15: Multiple Sclerosis

0.02 mg IL-4 and 200 IU IL-2 plus 0.001 μg BDNF and 200 IU IL-2 plus heat crème.

Patient 15 was at the beginning of the treatment a 43-year-old female, 60 kg, suffering from multiple sclerosis. The diagnosis of multiple sclerosis was confirmed in the year 2021 in accordance with code G35.10 of the standard ICD-10-GM 2021.

She reported sensory weakness in both legs, strong fatigue (sleeping more than 12 hours per day), strong listlessness, strong constipation and a strong urge to urinate at the latest 2 hours after the last urination. The patient described her condition as strongly affected by the disease.

Over the course of the treatment, the patient received no basic treatment due to refusal by the patient.

The treatment was performed as follows, wherein left and right forearm were treated at the same time:

Left forearm: About 50 mg to 800 mg Kytta® heat crème, usually about 40 mg to 150 mg, were applied topically on the skin of the upper side of the forearm to an area of about 25 cm² to 100 cm² as described in the Materials and Methods' section ‘Application of heat crème’. After about 5 min to 15 min the skin had turned red and warm and an increase in the skin temperature was palpable. Then, 100 μl of IL4-/IL-2 injection solution (containing 200 IU IL-2 and 0.02 mg IL-4) were intradermally injected within the skin of the red and warmed skin area.

Right forearm: Similarly, about 50 mg to 800 mg Kytta® heat crème, usually about 40 mg to 150 mg, were applied topically on the skin of the upper side of the forearm to an area of about 25 cm² to 100 cm² as described in the Materials and Methods' section ‘Application of heat crème’. After about 5 min to 15 min the skin had turned red and warm and an increase in the skin temperature was palpable. Then, 100 μl of BDNF/IL-2 injection solution (containing 200 IU IL-2 and 0.001 μg BDNF) were intradermally injected within the skin of the red and warmed skin area.

The injections were performed intrademally. The depth of the injections was about 1 mm to 2.5 mm underneath the surface of the skin. Following the injection, a characteristic and pronounced skin wheal was raised. The injection solutions were prepared as described in the Materials and Methods' section.

The treatment was performed every second day by the patient herself for one year.

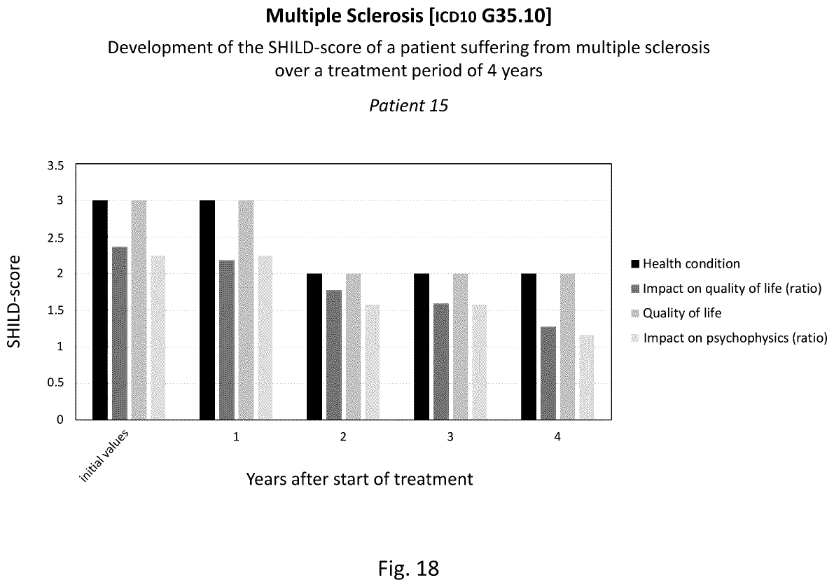

Since neither CRP nor HAQ nor BASDAI are decisive for multiple sclerosis, the SHILD questionnaire was used (c.f. Materials and Methods' section ‘SHIED—Questionnaire for multiple sclerosis’). The results are given in Table 12 and FIG. 18. It is pointed out that the positive effect of the treatment is reflected in the SHILD score typically only after a significant delay. Consequently, the SHILD score from year 2 after the start of treatment provides information about the success of the treatment in the first year after the start of treatment and shows a clear improvement. The results are given in Table 12 and FIG. 18.

Furthermore, the patient reported about a clear decrease in fatigue and listlessness, the time between the urinations increased and the obstipation was reduced within the first year after start of treatment. During the entire treatment from the start no single relapse had occurred while before the start of treatment usually one to four relapses per year were experienced by the patient. Neither adverse side effects nor intolerances nor incompatibilities were observed over the entire duration of the treatment.

Since BDNF was no longer available for the patient after one year of treatment, the treatment including BDNF was stopped and a follow-up treatment continued as outlined in Inventive Example 16.

### Inventive Example 16 (IE 16)—Patient 15: Multiple Sclerosis

0.02 mg IL-4 and 200 IU IL-2 Plus Heat Crème.

The patient, patient 15, was the same as in Inventive Example 15 suffering from multiple sclerosis in accordance with code G35.10 of the standard ICD-10-GM 2021. She received a follow-up treatment to the treatment of Inventive Example 15 as described herein. At the beginning of the treatment she was 44 years of age and weighed 60 kg.

The treatment was performed as described in Inventive Example 15, however, BDNF was not administered. Hence, the treatment was performed on the right or left forearm in the same manner as described in Inventive Example 15 for the left forearm. Since neither CRP nor HAQ nor BASDAI are decisive for multiple sclerosis, the SHILD questionnaire was used (c.f. Materials and Methods' section ‘SHIED—Questionnaire for multiple sclerosis’). As already stated above, the positive effect of the treatment is reflected in the SHILD score typically only after a significant delay. Consequently, the SHILD score from years 3 and 4 after the start of treatment provides information about the success of the treatment in the second and third year after the start of treatment and shows that the clear improvement in the SHILD achieved in year 2 by using could be maintained in years 3 and 4 without using BDNF. The results are given in Table 12 and FIG. 18. However, despite the fatigue was still reduced, the reduction was slightly less than when BDNF was additionally administered (c.f. Inventive Example 15).

Within the years of treatment, the patient showed increased strength and power. She was able to reduce her sleeping time to a minimum of 10 hours (before treatment 12 hours), the time between urination increased up to 4 hours and the constipation was reduced. Additionally, the patient reported to be more resistant to mental stress. Overall, the patient's health condition and quality of life improved over the years of treatment. Furthermore, during the treatment no single relapse had occurred while before the start of treatment usually one to four relapses per year were experienced by the patient. Neither adverse side effects nor intolerances nor incompatibilities were observed over the entire duration of the treatment.

Hence, patient 15 could be freed from relapses for a timespan of in total 4 years with the treatments according to Inventive Examples 15 and 16.

### Inventive Example 17 (IE 17)—Patient 1: Basedow's Disease

1000 IU IFN-γ (High Dose) and 200 IU IL-2 Plus Heat Crème

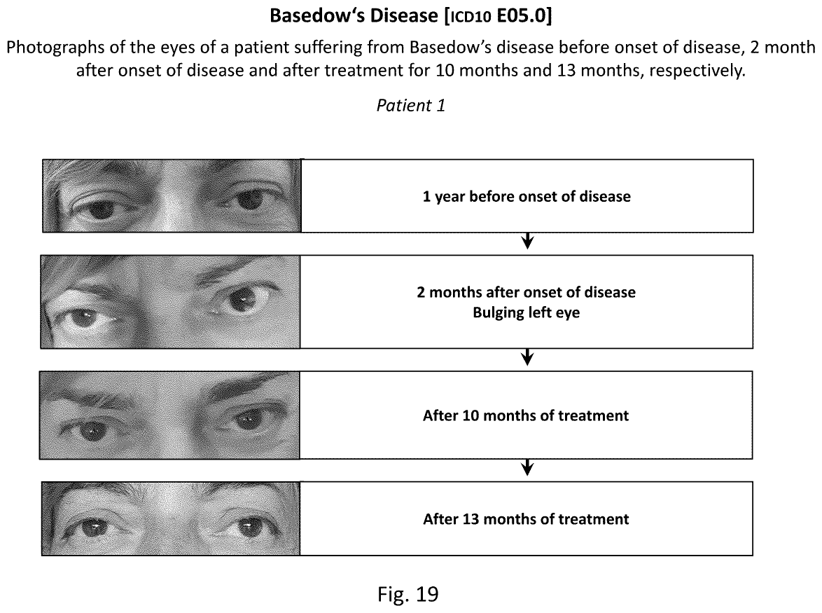

The patient (patient 1) was the same as in Inventive Example 1, a 46-year-old female weighing 58 kg. She suffered in addition to rheumatoid arthritis from Basedow's disease in accordance with code E05.0 of the standard ICD-10-GM 2021 for over 4 months. She showed a bulging eye before start of treatment (FIG. 19).

Over the course of the treatment, she received a constant basic treatment of 30 mg cortisone per day. This was necessary because the cortisol production by the adrenal glands of the patient was insufficient. The dose, composition and frequency of the cortisone basic treatment remained constant over the entire duration of the treatment.

The treatment was the same as described in Inventive Example 1, wherein the treatment regimen is indicated in Table 8A.

As can be seen from the photographs shown in FIG. 19, two months after onset of the disease clearly bulging eyes, particularly the left one, were observable. After 10 months of treatment according to the present invention, the bulging was reduced and scarcely detectable. Thirteen months after start of treatment, the eyes returned to their normal position in the socket, with nearly no difference detectable compared to the photographs taken before disease onset.

Overall, the patient reported an improvement and continued the treatment.

### Comparative Example 1 (CE 1)—Patient 1

Erroneous Treatment, Negative Control: Using Heat Crème Plus 200 IU IL-2 without IFN-γ on the Right Forearm and 1000 IU IFN-γ (High Dose) without Heat Crème Nor Heat Pad on the Left Forearm

Patient 1 of Inventive Example 1 experienced in addition to the treatment according to the present invention an erroneous or incorrect treatment according to a negative control. The patient was aware, informed and agreed on such treatment. The erroneous treatment was performed in week 10 after start of treatment and the non-effectiveness thereof was demonstrated with the results obtained in week 12 after treatment start (Table 8A and FIG. 10).

The erroneous treatment according to a negative control (Table 8A, indicated with [1]) was performed as follows:

Right forearm: On the upper side of the right forearm, a treatment with heat crème as described in Inventive Example 1 was performed. After about 15 min the skin had turned red and warm and an increase of the skin temperature was palpable. Then, 100 μl of IL-2 injection solution (containing 200 IU IL-2) were injected subcutaneously into a depth of about 2 mm to 4 mm underneath the surface of the skin within the red and warmed skin area. A characteristic skin wheal was raised.

Left forearm: On the upper side of the left forearm, solely 100 μl of high dose IFN-γ injection solution (containing 1000 IU IFN-γ) were injected subcutaneously into a depth of about 2 mm to 4 mm underneath the surface of the skin, wherein neither heat pad nor heat crème were used nor IL-2 administered. Again a characteristic skin wheal was raised by the injection. The injection in the left forearm was performed at about the same time as the injection in the right forearm (+/−2 min). No heat crème had been administered to and around the side of injection.

The injection solutions were prepared as described in the Materials and Methods' section.

The erroneous treatment was preceded by 4 weeks without treatment (weeks 6 to 9 after treatment start, Table 8A and FIG. 1) thereby re-establishing the initial untreated condition of a high CRP of 5 (Table 8A and FIG. 10, compare initial CRP and CRP at week 10 after start of treatment).

After the erroneous treatment had been performed, the patient reported that pain and fatigue had remained high and unchanged. The patient did also not report any health improvements or relieves from the disease over the following 1.5 weeks following the erroneous treatment as it was comparatively the case for the correct treatment of Inventive Example 1 after treatment start.

Hence, neither heat crème alone (i.e. neither generating an accumulation of PBMCs, vasodilation, increased blood volume, increased sO₂, increased rHb, increased temperature and/or redness alone) nor IL-2 alone nor the combination of both caused a beneficial effect. Furthermore, also the administration of even a high dose of 1000 IU IFN-γ alone cannot cause beneficial effects when no heat crème or heat is administered. In other words, the administration of even a high dose of 1000 IU IFN-γ is only effective when heat crème or heat is administered, i.e. an accumulation of PBMCs, vasodilation, increased blood volume, increased sO₂, increased rHb, increased temperature and/or redness is generated.

The patient also did not report any negative effects like adverse side effect nor intolerances nor incompatibilities. Therefore, it is further substantiated that the treatment and the substances used in the present invention indeed do not have any negative effects which however, might have been superimposed by the beneficial effects in the Inventive Examples.

### Comparative Example 2 (CE 2)—Patient 3

Erroneous Treatment, Negative Control Using a Heat Pad Plus IL-2 without IFN-γ; or IFN-γ (Low Dose of 100 IU) Alone without Heat Crème or Heat Pad

Patient 3 of Inventive Example 3 experienced in addition to the treatment according to the present invention an erroneous or incorrect treatment according to a negative control. The patient was aware, informed and agreed on such treatment. The erroneous treatment was performed as the first treatment (initial week) and the non-effectiveness thereof was confirmed with the results obtained in week 2 after the start of treatment (Table 8A).

The erroneous treatment according to a negative control (Table 8A, indicated with [2]) was performed as follows:

Right forearm: On the upper side of the right forearm, a treatment with a head pad as described in Inventive Example 3 was performed. About 1 min after removing the heat pad, 100 μl of IL-2 injection solution (containing 100 IU IL-2) were injected subcutaneously into a depth of about 2 mm to 4 mm underneath the surface of the skin. A characteristic skin wheal was raised.

Left forearm: On the upper side of the left forearm, 100 μl of low dose IFN-γ injection solution (containing 100 IU IFN-γ) were injected subcutaneously into a depth of about 2 mm to 4 mm underneath the surface of the skin, wherein neither heat pad nor heat crème were used nor IL-2 administered. Again a characteristic skin wheal was raised. No heat pad had been applied at and around the side of injection.

The injection solutions were prepared as described in the Materials and Methods' section.

The patient did not report any health improvements or relieves from the disease as it was comparatively the case for the correct treatments of Inventive Example 3, regardless whether including IL-2 or not (pain free, apart from lumbago, and reduced fatigue, at least for about 3 to 4 days after treatment).

Hence, neither heat pad alone (i.e. neither generating an accumulation of PBMCs, vasodilation, increased blood volume, increased sO₂, increased rHb, increased temperature and/or redness alone) nor IL-2 alone nor the combination of both caused a beneficial effect. Furthermore, also the administration of a low dose of 100 IU IFN-γ alone cannot cause beneficial effects when no heat crème or heat is applied. In other words, the administration of 100 IU IFN-γ acts synergistically together with the administration of heat crème or conditioning energy/heat, i.e. with the accumulation of PBMCs, vasodilation, increased blood volume, increased sO₂, increased rHb, increased temperature and/or redness is generated.

The patient also did not report about any negative effects like adverse side effect nor intolerances nor incompatibilities. Therefore, it is further substantiated that the treatment and substances as used in the present invention indeed do not have any negative effects which however, might have been superimposed by the beneficial effect in the Inventive Examples.

### Comparative Example 3 (CE 3)—Patient 1

Double Blinded Placebo Treatment Using Heat Crème about 1.5 Hours Before Administering 1000 IU IFN-γ (High Dose) and 200 IU IL-2

Patient 1 of Inventive Example 1 experienced in addition to the treatments according to the present invention and according to Comparative Example 1 a further incorrect treatment. Contrary to Comparative Example 1, both patient and the physician believed the conduction of the treatment had been done correctly (double blinded placebo). Therefore, a placebo effect could be excluded and the treatment was regarded as placebo treatment. The placebo treatment was performed over 5 weeks (Table 8A, weeks 12 to 16 after treatment start, and FIG. 10).

In the placebo treatment (Table 8A, indicated with [3]) the heat crème was applied correctly in the same way as described in Inventive Example 1. Furthermore, also the 100 μl of the high dose IFN-γ/IL-2 injection solution (containing 1000 IU IFN-γ and 200 IU IL-2) were prepared correctly and injected correctly in the same way as described in Inventive Example 1. However, the placebo treatment consisted in the fact that the injection of the IFN-γ/IL-2 injection solution was performed about 1 hour to 1.75 hours after the heat crème had been applied to the skin. Precisely, the patient already applied the cream at home and then made his way to the doctor. The injection was then administered at the doctor's office. The fluctuations in the CRP of the curve of FIG. 10 and in Table 8A can be explained by the fact that the journey to the doctor and/or the waiting time at the doctor's office sometimes took a little longer, sometimes a little shorter. Consequently, sometimes the effect of the cream may still have been sufficiently present at the time or beyond the time of the injection (values obtained in weeks 13 and 15 after start of treatment), sometimes it may have been not sufficiently present anymore (values obtained in weeks 12, 14, 16 and 17 after start of treatment). Depending on this, the treatment was successful or not.

Hence, the average CRP obtained from the weeks of incorrect treatment (CRP values of weeks 13 to 18 after the start of treatment) did not improve (FIG. 10, indicated trend). Similarly, also the joint condition did not improve during weeks 13 to 17 after start of treatment (Table 8A). As can be seen from FIG. 8 and as described in detail in the Materials and Methods' section ‘Thermal measurement and quantification of skin reddening’ in Reference Example 4, about 1 hour after the application of the heat crème at T(tc)₇, the skin temperature already started to return to its initial value and the reddening started to fade. About 1.5 hours after the application of the heat crème at T(tc)₈ the skin temperature had almost re-established to the initial value (FIG. 8) and the reddening was faded. The patient and the physician reported both that by the time the injection was performed the skin surface was no longer particularly red and/or an increased skin temperature was sometimes a little and sometimes not at all noticeable.

Hence, without the effect of the heat crème (i.e. without accumulation of PBMCs, vasodilation, increased blood volume, increased sO₂, increased rHb, increased temperature and/or redness) the beneficial effects were not achieved. The combination of IFN-γ and IL-2 in the indicated concentrations acts synergistically together with accumulation of PBMCs, vasodilation, increased blood volume, increased sO₂, increased rHb, increased temperature and/or redness to develop the beneficial effect.

Again, the patient did not report any negative effects like adverse side effect nor intolerances nor incompatibilities. Therefore, it is further substantiated that the treatment and substances used in the present invention indeed do not have any negative effects which however, might have been superimposed by the beneficial effect in the Inventive Examples.

### Comparative Example 4 (Patient 16)

Topical Administration of a High Amount of 20,000 IU IFN-γ in the Close Vicinity of an Inflamed Joint

Patient 16 was a 45 years old female, 65 kg, suffering from arthritis in the knees. She showed swelling of joints and joint pain in both knees. She was untreated with pain killers or any other medication. The patient was hardly able to walk down stairs forwardly under great pain. The patient described her condition as strongly affected since neither long walks nor sportive activities were possible.

An amount of 20,000 IU IFN-γ was topically administered twice daily (per day 40,000 IU IFN-γ in total) to each inflamed knee by using an IFN-γ containing crème. The crème was prepared as described in the Materials and Methods' section. Hence, an extremely high amount of IFN-γ was administered in close vicinity to a site of inflammation, i.e. close to antigen. An accumulation of PBMCs, vasodilation, increased blood volume, increased sO₂, increased rHb, increased temperature and/or redness was not induced nor a heat crème or heat pad applied.

The patient's condition was worsening over the treatment for three days, then the treatment was stopped.

While the Inventive Examples, Reference Examples and Comparative Examples of the present disclosure have been illustrated and described in detail in the figures and foregoing description, such illustrations and descriptions are to be considered illustrative or exemplary and not restrictive. The present disclosure is not limited to the disclosed embodiments. Other variations to the disclosed exemplary embodiments can be understood and effected by those skilled in the art in practicing the present disclosure provided herein, from a review of the figures, the disclosure, and the claims.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims. It is intended that the disclosure and examples be considered as exemplary only, with a true scope and spirit of disclosed embodiments being indicated by the following claims, along with the full scope of equivalents to which such claims are entitled. It is also to be understood that the terminology used herein is for the purpose of describing particular embodiments only, and is not intended to be limiting.

Annotation to the Tables 8A and B and 9 to 10: Patients were usually examined and treated in the same session. The success of the treatment or the effect thereof was then determined in the following session, usually one week later, and the next treatment performed as indicated in the tables.

TABLE 8A
Rheumatoid arthritis/Basedow's disease

| | Rheumatoid arthritis/Basedow's Disease | | | | | | | Rheumatoid arthritis | | | | | | |
|---|---|---|---|---|---|---|---|---|---|---|---|---|---|---|
| | [ICD-10 M05.80]/[ICD-10 E05.0] | | | | | | | [ICD-10 M05.80] | | | | | | |
| | IE 1/IE 17/CE 2/CE 3 (Patient 1) | | | | | | | IE 2 (Patient 2) | | | | | | |
| | [female/46 years/58 kg] | | | | | | | [female/59 years/62 kg] | | | | | | |
| | | | | | Treatment | | | | | | | Treatment | | |
| Weeks before | Results | | | | | | heat | Results | | | | | | heat |
| and after start | CRP | Tender | Swollen | | IFN-γ | IL-2 | crème/ | CRP | Tender | Swollen | | IFN-γ | IL-2 | crème/ |
| of treatment | [mg/l] | joints | joints | HAQ | [IU] | [IU] | pad | [mg/l] | joints | joints | HAQ | [IU] | [IU] | pad |
| −28 | 5 | — | — | — | —/— | —/— | —/— | | | | | | | |
| −1 | 6 | — | — | — | —/— | —/— | —/— | | | | | | | |
| init. | 5 | 17 | 2 | 1.25 | 1000 | 200 | c | 2.4 | 10 | 10 | 0.125 | 100 | —/— | p |
| 1 | 1 | 8 | 10 | 1 | 1000 | 200 | c | 0.6 | 10 | 10 | 0.125 | 100 | 200 | p |
| 2 | 4 | 13 | 10 | 0.75 | 1000 | 200 | c | 1.2 | 8 | 8 | 0 | 100 | 200 | p |
| 3 | 3 | 11 | 11 | 1.125 | 1000 | 200 | c | — | — | — | — | —/— | —/— | —/— |
| 4 | 2 | — | — | 1 | 1000 | 200 | c | — | — | — | — | —/— | —/— | —/— |
| 5 | 3 | — | — | 1.125 | 1000 | 200 | c | — | — | — | — | —/— | —/— | —/— |
| 6 | — | — | — | — | —/— | —/— | —/— | — | — | — | — | —/— | —/— | —/— |
| 7 | — | — | — | — | —/— | —/— | —/— | 0.6 | 3 | 3 | 0 | 100 | 200 | p |
| 8 | — | — | — | — | —/— | —/— | —/— | 0.6 | 8 | 8 | 0 | 100 | 200 | p |
| 9 | — | — | — | — | —/— | —/— | —/— | 0.7 | 8 | 8 | 0 | 100 | 200 | p |
| 10 | 5 | — | — | — | 1000 | 200 | c [1] | — | — | — | — | —/— | —/— | —/— |
| 11 | — | — | — | — | —/— | —/— | —/— | — | 0 | 0 | 0 | 100 | 200 | p |
| 12 | 5 | 5 | 1 | — | 1000 | 200 | c [3] | 0.6 | 0 | 0 | 0 | 100 | —/— | p |
| 13 | 4 | 4 | 3 | — | 1000 | 200 | c [3] | — | — | — | — | 100 | —/— | p |
| 14 | 6 | 2 | 5 | 1 | 1000 | 200 | c [3] | — | 0 | 0 | 0 | 100 | —/— | p |
| 15 | 3 | 4 | 2 | 1 | 1000 | 200 | c [3] | 0.7 | 0 | 0 | 0 | 100 | —/— | p |
| 16 | 6 | — | — | — | 1000 | 200 | c [3] | — | — | — | — | 100 | —/— | p |
| 17 | 6 | 4 | 8 | 1 | 1000 | 200 | c | — | — | — | — | 100 | —/— | p |
| 18 | 3 | 4 | 1 | 1 | 1000 | 200 | c | — | — | — | — | 100 | —/— | p |
| 19 | 2 | 3 | 1 | 1 | 1000 | 200 | c | — | — | — | — | —/— | —/— | —/— |
| 20 | 1 | 2 | 3 | 1 | 1000 | 200 | c | — | — | — | — | —/— | —/— | —/— |
| 21 | 1 | 0 | 0 | 1 | 1000 | 200 | c | — | — | — | — | —/— | —/— | —/— |
| 22 | 2 | 0 | 3 | 1 | 1000 | 200 | c | — | — | — | — | 100 | —/— | p |
| 23 | — | 3 | 3 | 1 | 1000 | 200 | c | — | — | — | — | —/— | —/— | —/— |
| 24 | 1 | 3 | 4 | 1 | 1000 | 200 | c | — | — | — | — | 100 | —/— | p |
| 25 | 1 | — | — | 1 | 1000 | 200 | c | — | — | — | — | 100 | —/— | p |
| 26 | 1 | — | — | — | 1000 | 200 | c | — | — | — | — | 100 | —/— | p |
| 27 | 1 | — | — | — | 1000 | 200 | c | — | — | — | — | 100 | —/— | p |
| 28 | 1 | 0 | 3 | 1 | 1000 | 200 | c | — | — | — | — | 100 | —/— | p |
| 29 | 1 | 3 | 3 | 1 | 1000 | 200 | c | — | — | — | — | 100 | —/— | p |
| 30 | — | 3 | 3 | 1 | 1000 | 200 | c | — | — | — | — | 100 | —/— | p |
| 31 | — | 2 | 3 | 1 | 1000 | 200 | c | — | — | — | — | 100 | —/— | p |
| 32 | — | — | — | — | —/— | —/— | —/— | — | — | — | — | 100 | —/— | p |
| 33 | — | — | — | — | —/— | —/— | —/— | — | 0 | 0 | 0 | 100 | —/— | p |
| 34 | — | — | — | — | —/— | —/— | —/— | — | — | — | — | 100 | —/— | p |
| 35 | 1 | — | — | 1 | 1000 | 200 | c | — | — | — | — | 100 | —/— | p |
| 36 | 1 | — | — | 1 | 1000 | 200 | c | — | — | — | — | 100 | —/— | p |
| 37 | 1 | — | — | 1 | 1000 | 200 | c | — | — | — | — | 100 | —/— | p |
| 38 | — | — | — | — | 1000 | 200 | c | — | 0 | 0 | 0 | 100 | —/— | p |
| 39 | — | — | — | — | —/— | —/— | —/— | — | — | — | — | 100 | —/— | p |
| 40 | — | — | — | 1 | 1000 | 200 | c | — | 0 | 0 | 0 | 100 | —/— | p |
| 41 | — | — | — | — | 1000 | 200 | c | <0.6 | 0 | 0 | 0 | 100 | —/— | p |
| 42 | — | — | — | — | 1000 | 200 | c | <0.6 | 0 | 5 | 0 | 100 | —/— | p |
| 43 | — | 3 | 3 | 1 | 1000 | 200 | c | — | — | — | — | 100 | —/— | p |
| 44 | — | — | — | — | 1000 | 200 | c | — | — | — | — | —/— | —/— | —/— |
| 45 | — | — | — | — | 1000 | 200 | c | — | 0 | 0 | 0 | 100 | —/— | p |
| 46 | — | — | — | 1 | 1000 | 200 | c | — | — | — | — | —/— | —/— | —/— |
| 47 | — | — | — | — | 1000 | 200 | c | — | — | — | — | 100 | —/— | p |
| 48 | — | — | — | — | 1000 | 200 | c | <0.6 | 0 | 0 | 0 | 100 | —/— | p |
| 49 | — | — | — | 1 | 1000 | 200 | c | — | — | — | — | 100 | —/— | p |
| 50 | — | — | — | — | —/— | —/— | —/— | <0.6 | 0 | 0 | 0 | 100 | —/— | p |
| 51 | 5 | 3 | 2 | 1 | 1000 | 200 | c | | | | | | | |
| 52 | 1 | 3 | 3 | 1 | 1000 | 200 | c | | | | | | | |
| 53 | 1 | — | — | 1 | 1000 | 200 | c | | | | | | | |
| | Rheumatoid arthritis | | | | | | | Rheumatoid arthritis | | | | | | |
| | [ICD-10 M05.80] | | | | | | | [ICD-10 M05.80] | | | | | | |
| | IE 3/CE 2 (Patient 3) | | | | | | | IE 4 (Patient 4) | | | | | | |
| | [female/83 years/78 kg] | | | | | | | [female/49 years/55 kg] | | | | | | |
| | | | | | Treatment | | | | | | | Treatment | | |
| Weeks before | Results | | | | | | heat | Results | | | | | | heat |
| and after start | CRP | Tender | Swollen | | IFN-γ | IL-2 | crème/ | CRP | Tender | Swollen | | IFN-γ | IL-2 | crème/ |
| of treatment | [mg/l] | joints | joints | HAQ | [IU] | [IU] | pad | [mg/l] | joints | joints | HAQ | [IU] | [IU] | pad |
| −28 | | | | | | | | | | | | | | |
| −1 | | | | | | | | | | | | | | |
| init. | 1.6 | 30 | 30 | 1.625 | 100 | 200 | p [2] | 1.89 | 14 | 11 | — | 1000 | 200 | c |
| 1 | 1.7 | 30 | 30 | 1.625 | 100 | —/— | p | 0.16 | 3 | 2 | — | 1000 | 200 | c |
| 2 | 1.7 | 30 | 30 | — | 100 | —/— | p | — | 3 | 2 | — | 1000 | 200 | c |
| 3 | — | — | — | — | 100 | —/— | p | — | — | — | — | —/— | —/— | —/— |
| 4 | — | — | — | — | 100 | —/— | p | — | — | — | — | —/— | —/— | —/— |
| 5 | — | — | — | — | 100 | —/— | p | — | 4 | 2 | — | 1000 | 200 | p |
| 6 | — | — | — | — | 100 | —/— | p | — | — | — | — | —/— | —/— | —/— |
| 7 | — | — | — | — | —/— | —/— | —/— | 0.32 | 4 | 2 | — | 1000 | 200 | p |
| 8 | 7.1* | 30 | 30 | 1 | 100 | 200 | p | — | — | — | — | —/— | —/— | —/— |
| 9 | 2.5* | — | — | — | 100 | 200 | p | — | 3 | 2 | — | 1000 | 200 | p |
| 10 | 2.1* | 20 | 20 | 1.125 | 100 | 200 | p | | | | | | | |
| 11 | — | — | — | 1.125 | 100 | 200 | p | | | | | | | |
| 12 | | | | | | | | | | | | | | |
| 13 | | | | | | | | | | | | | | |
| 14 | | | | | | | | | | | | | | |
| 15 | | | | | | | | | | | | | | |
| 16 | | | | | | | | | | | | | | |
| 17 | | | | | | | | | | | | | | |
| 18 | | | | | | | | | | | | | | |
| 19 | | | | | | | | | | | | | | |
| 20 | | | | | | | | | | | | | | |
| 21 | | | | | | | | | | | | | | |
| 22 | | | | | | | | | | | | | | |
| 23 | | | | | | | | | | | | | | |
| 24 | | | | | | | | | | | | | | |
| 25 | | | | | | | | | | | | | | |
| 26 | | | | | | | | | | | | | | |
| 27 | | | | | | | | | | | | | | |
| 28 | | | | | | | | | | | | | | |
| 29 | | | | | | | | | | | | | | |
| 30 | | | | | | | | | | | | | | |
| 31 | | | | | | | | | | | | | | |
| 32 | | | | | | | | | | | | | | |
| 33 | | | | | | | | | | | | | | |
| 34 | | | | | | | | | | | | | | |
| 35 | | | | | | | | | | | | | | |
| 36 | | | | | | | | | | | | | | |
| 37 | | | | | | | | | | | | | | |
| 38 | | | | | | | | | | | | | | |
| 39 | | | | | | | | | | | | | | |
| 40 | | | | | | | | | | | | | | |
| 41 | | | | | | | | | | | | | | |
| 42 | | | | | | | | | | | | | | |
| 43 | | | | | | | | | | | | | | |
| 44 | | | | | | | | | | | | | | |
| 45 | | | | | | | | | | | | | | |
| 46 | | | | | | | | | | | | | | |
| 47 | | | | | | | | | | | | | | |
| 48 | | | | | | | | | | | | | | |
| 49 | | | | | | | | | | | | | | |
| 50 | | | | | | | | | | | | | | |
| 51 | | | | | | | | | | | | | | |
| 52 | | | | | | | | | | | | | | |
| 53 | | | | | | | | | | | | | | |

[1] = Erroneous treatment, negative control (Patient 1, CE 1):
right arm: 200 IU IL-2 after heat crème application, no IFN-γ administered;
left arm: 1,000 IU IFN-γ, no heat crème application.
[2] = Erroneous treatment, negative control (Patient 3, CE 2):
right arm: 200 IU IL-2 after heat pad application, no IFN-γ administered;
left arm: 100 IU IFN-γ, no heat pad application.
[3] = Placebo treatment (Patient 1, CE 3): heat crème was applied about >1.5 hours before administering IFN-γ and IL-2, a heating of the skin was no longer noticeable (see also FIG. 8), no reddening was detectable. The attending physician and the patient assumed that the treatment had been carried out correctly.
*CRP is high due to an inflammation in a big toe joint resulting from mechanical overstrain non-related to RA. Hence, the CRP increase is presumably not related to RA and the CRP decrease from week 8 to 10 may not be related to the treatment but rather to a healing of overstrain.
init. = initial value and condition (untreated)/start with first treatment
p = heat pad
c = heat crème
—/— = no treatment/not administered
— = no data collected

TABLE 8B
Rheumatoid arthritis

| | Rheumatoid arthritis | | | | | | | Rheumatoid arthritis | | | | | | |
|---|---|---|---|---|---|---|---|---|---|---|---|---|---|---|
| | [ICD-10 M05.80] | | | | | | | [ICD-10 M05.80] | | | | | | |
| | IE 5 (Patient 5) | | | | | | | IE 6 (Patient 6) | | | | | | |
| | [female/57 years/75 kg] | | | | | | | [female/71 years/62 kg] | | | | | | |
| | | | | | Treatment | | | | | | | Treatment | | |
| Weeks after | Results | | | | | | heat | Results | | | | | | heat |
| start of | CRP | Tender | Swollen | | IFN-γ | IL-2 | crème/ | CRP | Tender | Swollen | | IFN-γ | IL-2 | crème/ |
| treatment | [mg/l] | joints | joints | HAQ | [IU] | [IU] | pad | [mg/l] | joints | joints | HAQ | [IU] | [IU] | pad |
| init. | 15.3 | 22 | — | 1.75 | 100 | —/— | p | 5.2 | 3 | 4 | 1.56 | 100 | —/— | p |
| 1 | — | — | 13 | 1.25 | 100 | —/— | p | 0.41 | 0 | 1 | 1.43 | 100 | —/— | p |
| 2 | 9.4 | 8 | | 1 | 100 | —/— | p | — | 0 | 2 | 1.44 | 100 | —/— | p |
| 3 | — | — | — | — | —/— | —/— | —/— | — | — | — | — | —/— | —/— | —/— |
| 4 | — | — | — | — | —/— | —/— | —/— | — | 2 | 2 | | —/— | —/— | —/— |
| 5 | 5.6 | 0 | 8 | 1.125 | 100 | —/— | p | 0.18 | — | 2 | — | 100 | —/— | p |
| 6 | 8.6 | 0 | 5 | 1.125 | 100 | —/— | p | — | 0 | 0 | 1.83 | 100 | —/— | p |
| 7 | 9.9 | 0 | 6 | 1.125 | 100 | —/— | p | — | 1 | 1 | 1.44 | 100 | —/— | p |
| 8 | — | 1 | 4 | 0.75 | 100 | —/— | p | 0.86 | 0 | 1 | 1.5 | 100 | —/— | p |
| 9 | 8.8 | 0 | 6 | 0.875 | 100 | —/— | p | | | | | | | |
| 10 | | | | | | | | | | | | | | |
| 11 | | | | | | | | | | | | | | |
| 12 | | | | | | | | | | | | | | |

| | Rheumatoid arthritis/Synovitis | | | | | | | |
|---|---|---|---|---|---|---|---|---|
| | [ICD-10 M05.80]/[ICD-10 M65.—] | | | | | | | |
| | IE 7 (Patient 7) | | | | | | | |
| | [female/55 years/48 kg] | | | | | | | Rheumatoid arthritis |
| | | | | | Treatment | | | [ICD-10 M05.80] |
| Weeks after | Results | | | | | | heat | IE 8 (Patient 8) |
| start of | CRP | Tender | Swollen | | IFN-γ | IL-2 | crème/ | [female/79 years/75 kg] |
| treatment | [mg/l]** | joints^{#} | joints^{#} | HAQ | [IU] | [IU] | pad | Treatment |
| init. | 0.03 | 5(10) | 6(10) | 1.75 | 1000 | 200 | p | Injection of isolated |
| 1 | — | 0(5) | 5(9) | — | 1000 | 200 | p | PBMCs and of 200 IU IL-2 |
| 2 | 0.09 | 1(4) | 4(4) | 1.375 | 1000 | 200 | p | with either 100 IU |
| 3 | 0.04 | 0(3) | 4(10) | — | 1000 | 200 | p | IFN-γ or 1000 IU IFN-γ. |
| 4 | — | 1(3) | 4(8) | — | 1000 | 200 | p | For further details, |
| 5 | — | — | — | — | —/— | —/— | —/— | please see specification, |
| 6 | — | 0 | 5(10) | 1.125 | 1000 | 200 | p | Inventive Example 8. |
| 7 | 0.03 | 7(0) | 8(4) | 1.25 | 1000 | 200 | p | |
| 8 | 0.03 | 0(5) | 4(10) | 1 | 1000 | 200 | p | |
| 9 | — | 0(8) | 4(8) | 1 | 1000 | 200 | p | |
| 10 | 0.03 | 3(10) | 0(10) | 1 | 1000 | 200 | p | |
| 11 | — | 0(0) | 0(10) | 0.875 | 1000 | 200 | p | |
| 12 | — | 0(10) | 1(10) | 0.875 | 1000 | 200 | p | |

init. = initial value and condition (untreated)/start with first treatment
p = heat pad
—/— = no treatment/not administered
— = no data collected
**Patient 7 received a basic treatment including an IL-6 inhibitor. The basic treatment was continued in parallel to the treatment in accordance with the present invention. This basic treatment accounts for the absence of elevated CRP values already before the start of treatment according to the present invention. The CRP in patient 7 has no significance for the effect of the treatment according to the present invention as long as the IL-6-inhibitor is being taken (see also specification).
^{#}Patient 7 showed for both forefeet strong pathologic deformations leading to swelling and pain non-attributable to the rheumatoid arthritis. The corresponding joints are indicated in italics and in brackets. The amount of swollen and tender joints related to rheumatoid arthritis are indicated in bold digits (see also specification).

TABLE 9
Polyarthritis

| | Polyarthritis | | | | | | | |
|---|---|---|---|---|---|---|---|---|
| | [ICD-10 M25.5 ] | | | | | | | |
| | IE 9 (Patient 9) | | | | | | | |
| | [female/77 years/58 kg] | | | | | | | Polyarthritis |
| | | | | | Treatment | | | [ICD-10 M25.5] |
| Weeks after | Results | | | | | | heat | IE 10 (Patient 10) |
| start of | CRP | Tender | Swollen | | IFN-γ | IL-2 | crème/ | [female/58 years/66 kg] |
| treatment | [mg/l] | joints | joints | HAQ | [IU] | [IU] | pad | Treatment |
| init. | 46.6 | 8 | 2 | 1.875 | 100 | —/— | p | Use of heat pad or |
| 1 | 25.4 | 8 | 0 | 1.875 | 100 | —/— | p | electrical hand warmer |
| 2 | 16.3 | 3 | 1 | 1.625 | 100 | —/— | p | plus and injection of |
| 3 | 11.3 | — | — | 1.625 | 100 | —/— | p | 200 IU and 1000 IU IFN-γ. |
| 4 | 10.7 | — | — | — | 100 | 200 | p | Later reheating of the |
| 5 | 5.4 | — | — | 0.75 | 100 | 200 | p | skin at and around the |
| 6 | — | — | — | 0.625 | 100 | 200 | p | site of injection. |
| 7 | — | — | — | — | 100 | 200 | p | For further details, |
| 8 | 3.8 | 1 | 0 | 0.625 | 100 | 200 | p | please see specification, |
| 9 | — | — | — | — | 100 | 200 | p | Inventive Example 10. |
| 10 | — | — | — | — | —/— | —/— | —/— | |
| 11 | — | — | — | — | —/— | —/— | —/— | |
| 12 | — | — | — | — | —/— | —/— | —/— | |
| 13 | — | — | — | — | 100 | 200 | p | |
| 14 | — | — | — | — | —/— | —/— | —/— | |
| 15 | — | — | — | — | 100 | 200 | p | |
| 16 | — | 0 | 0 | 0.375 | 100 | 200 | p | |
| 17 | 2.9 | — | — | — | 100 | 200 | p | |
| 18 | — | — | — | — | 100 | 200 | p | |
| 19 | 2.9 | 0 | 0 | 0.375 | 100 | 200 | p | |

init. = initial value and condition (untreated)/start with first treatment
p = heat pad
—/— = no treatment/not administered
— = no data collected

TABLE 10
Ankylosing spondylitis

| | Ankylosing spondylitis | | | | | | Ankylosing spondylitis | | | | | |
|---|---|---|---|---|---|---|---|---|---|---|---|---|
| | IE 11 (Patient 11) | | | | | | IE 12 (Patient 12) | | | | | |
| | [female/55 years/86 kg] | | | | | | [male/42 years/93 kg] | | | | | |
| | | | | Treatment | | | | | | Treatment | | |
| Weeks after | Results | | | | | heat | Results | | | | | heat |
| start of | | Tender | Swollen | IFN-γ | IL-2 | crème/ | | Tender | Swollen | IFN-γ | IL-2 | crème/ |
| treatment | BASDAI | joints | joints | [IU] | [IU] | pad | BASDAI | joints | joints | [IU] | [IU] | pad |
| init. | — | 7 | 5 | 1000 | 200 | p | 5 | — | — | 100 | —/— | p |
| 1 | — | 14 | 5 | 1000 | 200 | p | — | — | — | 100 | —/— | p |
| 2 | — | 14 | 5 | 1000 | 200 | p | 4.3 | — | — | 100 | —/— | p |
| 3 | 6.45 | 5 | 6 | 1000 | 200 | p | 4 | — | — | 100 | —/— | p |
| 4 | — | — | — | —/— | —/— | —/— | — | — | — | 100 | —/— | p |
| 5 | — | — | — | —/— | —/— | —/— | — | — | — | 100 | —/— | p |
| 6 | 5.75 | 8 | 6 | 1000 | 200 | p | — | — | — | 100 | —/— | p |
| 7 | — | — | — | —/— | —/— | —/— | — | — | — | 100 | —/— | p |
| 8 | 5.75 | 8 | 5 | 1000 | 200 | p | — | — | — | 100 | —/— | p |
| 9 | — | — | — | —/— | —/— | —/— | — | — | — | 100 | —/— | p |
| 10 | 4.6 | 3 | 3 | 1000 | 200 | p | 3 | — | — | 100 | —/— | p |

init. = initial value and condition (untreated)/start with first treatment
p = heat pad
—/— = no treatment/not administered
— = no data collected

TABLE 11
Polymyalgia rheumatica

| | Polymyalgia rheumatica | | | | | | | Polymyalgia rheumatica | | | | | | |
|---|---|---|---|---|---|---|---|---|---|---|---|---|---|---|
| | [ICD-10 M35.3] | | | | | | | [ICD-10 M35.3] | | | | | | |
| | IE 13 (Patient 13) | | | | | | | IE 14 (Patient 14) | | | | | | |
| | [female/57 years/78 kg] | | | | | | | [male/66 years/90 kg] | | | | | | |
| Weeks [plus | | | | | Treatment | | | | | | | Treatment | | |
| days] after | Results | | | | | | heat | Results | | | | | | heat |
| start of | CRP | Tender | Swollen | | IFN-γ | IL-2 | crème/ | CRP | Tender | Swollen | | IFN-γ | IL-2 | crème/ |
| treatment | [mg/l] | joints | joints | HAQ | [IU] | [IU] | pad | [mg/l] | joints | joints | HAQ | [IU] | [IU] | pad |
| init. | 2.5 | 35 | 25 | 1.625 | 100 | —/— | p | 2.4 | 34 | 30 | 0.75 | 100 | 0 | p |
| 1 | 3 | 52 | 52 | 1.5 | 2 × 100 | —/— | p | — | — | — | — | —/— | —/— | —/— |
| 2 | — | — | — | — | —/— | —/— | —/— | 3.4 | 19 | 15 | 0.75 | 1000 | 200 | p |
| 2[3] | 3.5 | 26 | 0 | 1.5 | 100 | 200 | p | — | — | — | — | —/— | —/— | —/— |
| 3 | — | — | — | — | —/— | —/— | —/— | 1.9 | — | — | — | 1000 | 200 | p |
| 3[3] | 3 | 4 | 30 | 1.625 | 1000 | 200 | p | 1.1 | 17 | 17 | 0.875 | 1000 | 200 | p |
| 4 | — | — | — | — | —/— | —/— | —/— | — | — | — | — | 1000 | 200 | p |
| 4[3] | — | — | — | — | 1000 | 200 | p | — | — | — | — | 1000 | 200 | p |
| 5 | — | — | — | — | 1000 | 200 | p | 1.5 | 15 | 15 | 0.75 | 1000 | 200 | p |
| 5[3] | — | — | — | — | —/— | —/— | —/— | — | — | — | — | 1000 | 200 | p |
| 6 | — | — | — | — | —/— | —/— | —/— | | | | | | | |
| 6[3] | 8.8 | 6 | 0 | 1.625 | 1000 | 200 | p | | | | | | | |
| 7 | — | — | — | — | 1000 | 200 | p | | | | | | | |
| 7[3] | 7 | 50 | 30 | 1.5 | 1000 | 200 | p | | | | | | | |
| 8 | — | — | — | — | 1000 | 200 | p | | | | | | | |
| 8[3] | — | — | — | — | 1000 | 200 | p | | | | | | | |
| 9 | 6.5 | 4 | 30 | 1.135 | 1000 | 200 | p | | | | | | | |

init. = initial value and condition (untreated)/start with first treatment
p = heat pad
—/— = no treatment/not administered
— = no data collected
For instance, ‘2[3]’ means ‘2 weeks and 3 days’ after start of treatment

TABLE 12
Multiple sclerosis

| | Multiple sclerosis [ICD-10 G35.10] | | | |
|---|---|---|---|---|
| | IE 15/IE 16 (Patient 15) [female/43 years/62 kg] | | | |
| | Results | | | |
| Years after | | Impact on | | Impact on |
| start of | Health | quality of life | Quality | psychophysics |
| treatment | condition | U(ratio) | of life | (ratio) |
| init. | 3 | 2.36 | 3 | 2.25 |
| 1 | 3 | 2.18 | 3 | 2.25 |
| 2 | 2 | 1.77 | 2 | 1.583 |
| 3 | 2 | 1.59 | 2 | 1.583 |
| 4 | 2 | 1.27 | 2 | 1.167 |

init. = initial value and condition (untreated)/start with treatment

### CLAUSES

1. Method for treating and/or preventing an inflammatory disease, immunological disease and/or autoimmunological disease in a subject,
wherein the method comprises a step (A), wherein step (A) is selected from one or more of steps:
   (A-0) generating an accumulation of PBMCs (peripheral blood mononuclear cells) within the skin of the subject; and/or
   (A-1) generating a vasodilation of the capillaries within the skin of the subject; and/or
   (A-2) generating an increased blood volume within the skin of the subject; and/or
   (A-3) generating an increased sO₂ (oxygen saturation of haemoglobin) within the skin and/or an increased rHb (relative haemoglobin amount) within the skin of the subject; and/or
   (A-4) generating an increased temperature on the skin of the subject; and/or
   (A-5) generating a redness on the skin of the subject; and/or
   (A-6) administering conditioning energy to the skin of the subject; and/or
   (A-7) administering a skin-conditioning agent to the skin of the subject; and/or
   (A-8) administering PBMCs (peripheral blood mononuclear cells) into the skin of the subject,
and the method further comprises the step(s) of:
   (B) administering an immunomodulatory substance(s) to the skin of the subject; and/or
   (C) administering an immunomodulatory substance(s) to the skin of the subject,
   wherein immunomodulatory substance(s) administered in step (C) is different from the immunomodulatory substance(s) administered in step (B).

2. Method according to clause 1, wherein the skin is of a skin area, wherein preferably the skin of the skin area is immunological inactive and/or unchallenged and/or spatially distanced to any site of immunological activity and/or challenge.

3. Method according to any of the preceding clauses, wherein the immunomodulatory substance(s) is administered in step (B) and/or step (C) in an amount that does not cause a systemic increase of the concentration of the immunomodulatory substance(s) in the subject.

4. Method according to any of the preceding clauses, wherein step (A) is selected from one or more of steps (A-0), (A-1), (A-2), (A-3) and/or (A-4).

5. Method according to any of the preceding clauses, wherein in step (A-1) of the method:
the vasodilation is determined in terms of the sO₂ and/or the rHb within the skin.

6. Method according to any of the preceding clauses, wherein in step (A-2) of the method:
the increased blood volume is determined in terms of the sO₂ and/or the rHb within the skin.

7. Method according to any of the preceding clauses, wherein in step (A-3) of the method:
the sO₂ is increased by 2% or more and/or the sO₂ is increased by 2%-points or more; and/or the rHb is increased by 2% or more and/or the rHb is increased by 2 AU (arbitrary units).

8. Method according to any of the preceding clauses, wherein in step (A-4) of the method:
the temperature is increased by 1% or more and/or the temperature is increased by 0.2° C. or more.

9. Method according to any of the preceding clauses, wherein in step (A-6) of the method the conditioning energy is administered to generate:
a vasodilation of the capillaries within the skin of the subject, wherein the vasodilation is determined in terms of the sO₂ and/or the rHb; and/or
an increased blood volume within the skin of the subject, wherein the vasodilation is determined in terms of the sO₂ and/or the rHb; and/or
an increased sO₂ and/or an increased rHb within the skin of the subject, wherein preferably the sO₂ is increased by 2% or more and/or increased by 2%-points or more; and/or the rHb is increased by 2% or more and/or by 2 AU (arbitrary units) or more; and/or
an increased temperature on the skin of the subject, wherein preferably the temperature is increased by 1% or more and/or is increased by 0.2° C. or more; and/or
a redness on the skin of the subject, wherein preferably the redness is visually detectable to the a naked human eye,
wherein preferably the conditioning energy is administered by using an energizing means.

10. Method according to any of the preceding clauses, wherein in (A-7) of the method the skin-conditioning agent is administered to generate:
a vasodilation of the capillaries within the skin of the subject, wherein the vasodilation is determined in terms of the sO₂ and/or the rHb; and/or
an increased blood volume within the skin of the subject, wherein the vasodilation is determined in terms of the sO₂ and/or the rHb; and/or
an increased sO₂ and/or an increased rHb within the skin of the subject, wherein preferably the sO₂ is increased by 2% or more and/or increased by 2%-points or more; and/or the rHb is increased by 2% or more and/or by 2 AU (arbitrary units) or more; and/or
an increased temperature on the skin of the subject, wherein preferably the temperature is increased by 1% or more and/or is increased by 0.2° C. or more; and/or
a redness on the skin of the subject, wherein preferably the redness is visually detectable to the a naked human eye.

11. Method according to any of the preceding clauses, wherein in step (A-8) of the method the PBMCs are injected into a sub-topical layer of the skin.

12. Method according to any of the preceding clauses, wherein the immunomodulatory substance(s) of step (B) and/or (C) is a cytokine-like acting substance(s), preferably an interferon-like acting substance(s), interleukin-like acting substance(s) and/or neurotrophin-like acting substance(s), more preferably an IFN-γ-like acting substance(s), IL-4-like acting substance(s), BDNF-like acting substance(s) and/or IL-2 like acting substance(s), even more preferably IFN-γ (interferon-gamma), IL-4 (interleukin 4), BDNF (brain-derived neurotrophic factor) and/or IL-2 (interleukin 2), or any derivative, fragment, protein-analogue, peptide, protein-variant, biopharmaceutical, inductor, precursor, prodrug, mutein, co-drug, propeptide thereof and/or any pharmaceutically acceptable salt of any of these, still more preferably IFN-γ, IL-4, BDNF and/or IL-2.

13. Method according to any of the preceding clauses, wherein:
the method comprises steps (A) and (B); and
the immunomodulatory substance(s) administered in step (B) is IFN-γ, IL-4 and/or BDNF,
or
the method comprises steps (A), (B) and (C);
the immunomodulatory substance(s) in step (B) is any of the immunomodulatory substance(s) except for IL-2; and
the immunomodulatory substance(s) in step (C) is IL-2,
or
the method comprises steps (A), (B) and (C);
the immunomodulatory substance(s) in step (B) is IFN-γ, IL-4 and/or BDNF; and
the immunomodulatory substance(s) in step (C) is IL-2.

14. Method according to any of the preceding clauses, wherein:
the method comprises steps (A), (B) and optionally (C);
the immunomodulatory substance(s) in step (B) is IFN-γ; and
the immunomodulatory substance(s) in step (C) is IL-2,
or
the method comprises steps (A), (B) and optionally (C);
the immunomodulatory substance(s) in step (B) is IL-4 and/or BDNF, preferably IL-4; and
the immunomodulatory substance(s) in step (C) is IL-2.

15. Method according to clauses 12 to 14, wherein:
the IFN-γ is administered in an amount of 600 IU/kg body mass of the subject or less; in an amount of 30 ng/kg body mass of the subject or less; in a total amount of 30,000 IU or less; and/or in a total amount of 1,500 ng or less;
the IL-4 is administered in an amount of 20 ng/kg body mass of the subject or less; and/or in a total amount of 1,000 ng or less;
the BDNF is administered in an amount of 10 ng/kg per body mass of the subject or less; and/or in a total amount of 500 ng or less; and/or
the IL-2 is administered in an amount of 10 IU/kg body mass of the subject or less; in an amount of 0.6 ng/kg body mass of the subject or less; in a total amount of 500 IU or less; and/or in a total amount of 30.5 ng or less.

16. Method according to any of the preceding clauses, wherein the inflammatory disease, immunological disease and/or autoimmunological disease to be treated and/or prevented comprises, preferably consists of, arthritis, synovitis, tenosynovitis, inflammatory rheumatic disease, Hashimoto's disease, Basedow's disease, Morbus Crohn and/or multiple sclerosis.

17. Method according to any of the preceding clauses, wherein
the method comprises steps (A), (B) and (C);
the inflammatory disease, immunological disease and/or autoimmunological disease to be treated and/or prevented is any of the inflammatory disease, immunological disease and/or autoimmunological disease to be treated and/or prevented;
the immunomodulatory substance(s) in step (B) is the immunomodulatory substance(s) except for IL-2; and
the immunomodulatory substance(s) in step (C) is IL-2,
or
the method comprises steps (A) and (B) and optionally (C);
the inflammatory disease, immunological disease and/or autoimmunological disease to be treated and/or prevented is any of the inflammatory disease, immunological disease and/or autoimmunological disease to be treated and/or prevented except for an inflammatory disease of the nervous system, preferably except for multiple sclerosis, preferably is arthritis, synovitis, tenosynovitis, inflammatory rheumatic disease, Hashimoto's disease and/or Basedow's disease, more preferably is arthritis, synovitis, tenosynovitis, rheumatoid arthritis (RA), psoriatic arthritis, polymyalgia rheumatica, Bechterew's disease and/or Basedow's disease;
the immunomodulatory substance(s) in step (B) is IFN-γ and/or IL-4, preferably is IFN-γ; and
the immunomodulatory substance(s) in step (C) is IL-2,
or
the method comprises steps (A) and (B) and optionally (C);
the inflammatory disease, immunological disease and/or autoimmunological disease to be treated and/or prevented is an inflammatory disease of the nervous system, preferably is multiple sclerosis;
the immunomodulatory substance(s) in step (B) is IL-4 and/or BDNF, preferably IL-4; and
the immunomodulatory substance(s) in step (C) is IL-2.

18. Method in a method for treating and/or preventing an inflammatory disease, immunological disease and/or autoimmunological disease in a subject, wherein the skin-conditioning agent of step (A-7) of the method is a blood-circulation-increasing agent, vasodilating agent, skin-temperature increasing agent, skin-502-increasing agent and/or skin-rHb-increasing agent, preferably the skin-conditioning agent comprises nitrates, alpha blockers, ACE-inhibitors, ginkgo preparations like gingko balm, calcium antagonists, dihydralazine, minoxidil, dihydroergotoxin, nicotinic acid analogues, vasodilators like methylnicotinat, moxa herbs, capsaicine, pentoxifylline, vasodilator containing crème, methylnicotinat containing crème, Kytta® heat balm and/or any combination thereof.

19. Method according to any of the preceding clauses, wherein step (A), step (B) and/or step (C) is performed multiple times.

20. Method according to any of the preceding clauses, wherein the administration of steps (B) and/or (C) is effected partially or totally in the same area of the skin where the generating and/or administering of step (A) is effected.

21. Method according to any of the preceding clauses, wherein the method is performed in such a way that the individual effects or generated by step (A), (B), (B₁) and/or (C) at least partially and/or totally overlap in time and spatially.

22. Method according to any of the preceding clauses, wherein the immunomodulatory substance(s) are administered in combination with each other.

23. Method according to any of the preceding clauses, wherein the immunomodulatory substance(s) are administered separately in time.

24. Method according to any of the preceding clauses, wherein the method is performed within 5 hours or less.

25. Method according to any of the preceding clauses, wherein the administration in steps (A-6), (A-7) and/or (A-8) is by topical application or by injection.

26. Method according to any of the preceding clauses, wherein the immunomodulatory substance(s) are administered by topical application or by injection.

27. Method according to any of the preceding clauses, wherein the immunomodulatory substance(s) causes the PBMCs to mature, differentiate and/or proliferate PBMCs.

28. Method according to any of the preceding clauses, wherein the subject is a mammal or human.

### LIST OF REFERENCE SIGNS

100 medical device
102 skin
110 skin-conditioning unit
112 heating unit
114 contact detection means
116 wireless coupling means
118 energy source
119 trigger
120 applicator unit
121 first reservoir
122 actuator, pump
123 control unit
126 first applicator
127 applicator actuator
130 controller
132 control unit
200 handle, housing
601 coupling
602 coupling
603 coupling
604 coupling
605 coupling
606 coupling
607 coupling
608 coupling
701 external connector
702 user display
703 wireless coupling means
704 storage area

1. A method for treating and/or preventing an inflammatory disease, immunological disease and/or autoimmunological disease in a subject,
wherein the method comprises a step (A), wherein step (A) is selected from one or more of steps:
(A-0) generating an accumulation of PBMCs (peripheral blood mononuclear cells) within the skin of the subject;
(A-1) generating a vasodilation of the capillaries within the skin of the subject;
(A-2) generating an increased blood volume within the skin of the subject;
(A-3) generating an increased sO₂ (oxygen saturation of haemoglobin) within the skin and/or an increased rHb (relative haemoglobin amount) within the skin of the subject;
(A-4) generating an increased temperature on the skin of the subject;
(A-5) generating a redness on the skin of the subject;
(A-6) administering conditioning energy to the skin of the subject;
(A-7) administering a skin-conditioning agent to the skin of the subject; and/or
(A-8) administering PBMCs into the skin of the subject,
and the method further comprises the step(s) of:
(B) administering an immunomodulatory substance(s) to the skin of the subject; and/or
(C) administering an immunomodulatory substance(s) to the skin of the subject,
wherein immunomodulatory substance(s) administered in step (C) is different from the immunomodulatory substance(s) administered in step (B).

2. The method according to claim 1, wherein the skin is of a skin area, wherein preferably the skin of the skin area is immunological inactive and/or unchallenged and/or spatially distanced to any site of immunological activity and/or challenge.

3. The method of claim 1, wherein the immunomodulatory substance(s) is administered in step (B) and/or step (C) in an amount that does not cause a systemic increase of the concentration of the immunomodulatory substance(s) in the subject.

4. The method of claim 1, wherein step (A) is selected from one or more of steps (A-0), (A-1), (A-2), (A-3) and/or (A-4).

5. The method of claim 1, wherein in step (A-1) of the method:
the vasodilation is determined in terms of the sO₂ and/or the rHb within the skin.

6. The method of claim 1, wherein in step (A-2) of the method:
the increased blood volume is determined in terms of the sO₂ and/or the rHb within the skin.

7. The method of claim 1, wherein in step (A-3) of the method:
the sO₂ is increased by 2% or more and/or the sO₂ is increased by 2%-points or more; and/or the rHb is increased by 2% or more and/or the rHb is increased by 2 AU (arbitrary units).

8. The method of claim 1, wherein in step (A-4) of the method:
the temperature is increased by 1% or more and/or the temperature is increased by 0.2° C. or more.

9. The method of claim 1, wherein in step (A-6) of the method the conditioning energy is administered to generate:
a vasodilation of the capillaries within the skin of the subject, wherein the vasodilation is determined in terms of the sO₂ and/or the rHb; and/or
an increased blood volume within the skin of the subject, wherein the vasodilation is determined in terms of the sO₂ and/or the rHb; and/or
an increased sO₂ and/or an increased rHb within the skin of the subject, wherein preferably the sO₂ is increased by 2% or more and/or increased by 2%-points or more; and/or the rHb is increased by 2% or more and/or by 2 AU (arbitrary 50 units) or more; and/or
an increased temperature on the skin of the subject, wherein preferably the temperature is increased by 1% or more and/or is increased by 0.2° C. or more; and/or
a redness on the skin of the subject,
wherein preferably the conditioning energy is administered by using an energizing means.

10. The method of claim 1, wherein in step (A-7) of the method the skin-conditioning agent is administered to generate:
a vasodilation of the capillaries within the skin of the subject, wherein the vasodilation is determined in terms of the sO₂ and/or the rHb; and/or
an increased blood volume within the skin of the subject, wherein the vasodilation is determined in terms of the sO₂ and/or
the rHb; and/or
an increased sO₂ and/or an increased rHb within the skin of the subject, wherein preferably the sO₂ is increased by 2% or more and/or increased by 2%-points or more; and/or the rHb is increased by 2% or more and/or by 2 AU (arbitrary units) or more; and/or
an increased temperature on the skin of the subject, wherein preferably the temperature is increased by 1% or more and/or is increased by 0.2° C. or more; and/or
a redness on the skin of the subject.

11. The method of claim 1, wherein in step (A-8) of the method the PBMCs are injected into a sub-topical layer of the skin.

12. The method of claim 1, wherein the immunomodulatory substance(s) of step (B) and/or (C) is a cytokine-like acting substance(s).

13. The method of claim 1, wherein:
the method comprises steps (A) and (B);
the immunomodulatory substance(s) for use is IFN-γ, IL-4 and/or BDNF; and
the immunomodulatory substance(s) administered in step (B) is IFN-γ, IL-4 and/or BDNF,
or
the method comprises steps (A), (B) and (C);
the immunomodulatory substance(s) for use any of the immunomodulatory substance(s);
the immunomodulatory substance(s) in step (B) is any of the immunomodulatory substance(s) except for IL-2; and
the immunomodulatory substance(s) in step (C) is IL-2,
or
the method comprises steps (A), (B) and (C);
the immunomodulatory substance(s) for use is IFN-γ, IL-4, BDNF and/or IL-2;
the immunomodulatory substance(s) in step (B) is IFN-γ, IL-4 and/or BDNF; and
the immunomodulatory substance(s) in step (C) is IL-2.

14. The method of claim 1, wherein:
the method comprises steps (A), (B) and optionally (C);
the immunomodulatory substance(s) for use is IFN-γ and/or IL-2;
the immunomodulatory substance(s) in step (B) is IFN-γ; and
the immunomodulatory substance(s) in step (C) is IL-2,
or
the method comprises steps (A), (B) and optionally (C);
the immunomodulatory substance(s) for use is IL-4, BDNF and/or IL-2;
the immunomodulatory substance(s) in step (B) is IL-4 and/or BDNF; and
the immunomodulatory substance(s) in step (C) is IL-2.

15. The method of claim 13, wherein:
the IFN-γ is administered in an amount of 600 IU/kg body mass of the subject or less; in an amount of 30 ng/kg body mass of the subject or less; in a total amount of 30,000 IU or less; and/or in a total amount of 1,500 ng or less;
the IL-4 is administered in an amount of 20 ng/kg body mass of the subject or less; and/or in a total amount of 1,000 ng or less;
the BDNF is administered in an amount of 10 ng/kg per body mass of the subject or less; and/or in a total amount of 500 ng or less; and/or
the IL-2 is administered in an amount of 10 IU/kg body mass of the subject or less; in an amount of 0.6 ng/kg body mass of the subject or less; in a total amount of 500 IU or less; and/or in a total amount of 30.5 ng or less.

16. The method of claim 1, wherein the inflammatory disease, immunological disease and/or autoimmunological disease to be treated and/or prevented comprises, preferably consists of, arthritis, synovitis, tenosynovitis, inflammatory rheumatic disease, Hashimoto's disease, Basedow's disease, Morbus Crohn and/or multiple sclerosis.

17. The method of claim 1, wherein
the method comprises steps (A), (B) and (C);
the immunomodulatory substance(s) for use is any immunomodulatory substance(s);
the immunomodulatory substance(s) in step (B) is any immunomodulatory substance(s) except for IL-2; and
the immunomodulatory substance(s) in step (C) is IL-2,
or
the method comprises steps (A) and (B) and optionally (C);
the immunomodulatory substance(s) for use is IFN-γ, IL-4 and/or IL-2;
the inflammatory disease, immunological disease and/or autoimmunological disease to be treated and/or prevented is any inflammatory disease, immunological disease and/or autoimmunological disease except for an inflammatory disease of the nervous system;
the immunomodulatory substance(s) in step (B) is IFN-γ and/or IL-4; and
the immunomodulatory substance(s) in step (C) is IL-2,
or
the method comprises steps (A) and (B) and optionally (C);
the immunomodulatory substance(s) for use is IL-4, BDNF and/or IL-2;
the inflammatory disease, immunological disease and/or autoimmunological disease to be treated and/or prevented is an inflammatory disease of the nervous system;
the immunomodulatory substance(s) in step (B) is IL-4 and/or BDNF; and
the immunomodulatory substance(s) in step (C) is IL-2.

18. The method of claim 1, wherein the skin-conditioning agent for use and the skin-conditioning agent of step (A-7) of the method is a blood-circulation-increasing agent, vasodilating agent, skin-temperature increasing agent, skin-sO₂-increasing agent and/or skin-rHb-increasing agent, and/or any combination thereof.

19. The method of claim 1, wherein step (A), step (B) and/or step (C) is performed multiple times.

20. Pharmaceutical composition (I) comprising:
a skin-conditioning agent; and
an immunomodulatory substance(s).

21. Pharmaceutical composition (II) comprising:
an IFN-γ-like acting substance(s);
an IL-4-like acting substance(s);
an BDNF-like acting substance(s); and/or
an IL-2-like acting substance(s),
wherein the pharmaceutical composition is suitable to deliver:
IFN-γ-like acting substance(s)
in an amount equivalent to 600 IU IFN-γ/kg body mass of the subject or less;
in an amount equivalent in activity to 30 ng IFN-γ/kg body mass of the subject or less;
50 in a total amount equivalent to 30,000 IU IFN-γ or less; and/or
in a total amount equivalent in activity to 1,500 ng IFN-γ or less;
IL-4-like acting substance(s)
in an amount equivalent in activity to 20 ng IL-4/kg body mass of the subject or less; and/or
in a total amount equivalent in activity to 1,000 ng IL-4 or less;
BDNF-like acting substance(s)
in an amount equivalent in activity to 10 ng BDNF/kg per body mass of the subject or less; and/or
in a total amount equivalent in activity to 500 ng BDNF or less; and/or
IL-2-like acting substance(s) in an amount equivalent to 10 IU/kg body mass of the subject or less;
in an amount of 0.6 ng/kg body mass of the subject or less;
in a total amount equivalent in activity to 500 IU IL-2 or less; and/or
in a total amount equivalent in activity to 30.5 ng IL-2 or less.

22. Pharmaceutical composition (II) according to claim 21 comprising:
IFN-γ;
IL-4;
BDNF; and/or
IL-2,
wherein the pharmaceutical composition is suitable to deliver:
IFN-γ in an amount of 600 IU/kg body mass of the subject or less; in an amount of 30 ng/kg body mass of the subject or less; in a total amount of 30,000 IU or less; and/or in a total amount of 1,500 ng or less;
IL-4 in an amount of 20 ng/kg body mass of the subject or less; and/or in a total amount of 1,000 ng or less;
BDNF in an amount of 10 ng/kg per body mass of the subject or less; and/or in a total amount of 500 ng or less; and/or
IL-2 in an amount of 10 IU/kg body mass of the subject or less; in an amount of 0.6 ng/kg body mass of the subject or less; in a total amount of 500 IU or less; and/or in a total amount of 30.5 ng or less.

23. Pharmaceutical composition (111), comprising:
IFN-γ-like acting substance(s) in a concentration equivalent to 100,000 IU IFN-γ/ml or IU IFN-γ/g or less, or in a concentration equivalent in activity to 5,000 ng IFN-γ/ml or ng IFN-γ/g or less;
IL-4-like acting substance(s) in a concentration or equivalent in activity to 1,000 ng IL-4/ml or ng/g or less;
BDNF-like acting substance(s) in a concentration equivalent in activity to 1,500 ng BDNF/ml or ng/g or less;
IL-2-like acting substance(s) in a concentration equivalent to 100,000 IU IL-2/ml or IU IL-2/g less, or in a concentration equivalent in activity to 6100 ng IL-2/ml or ng IL-2/g or less.

24. Pharmaceutical composition (111) according to claim 23, comprising:
IFN-γ in a concentration of 100,000 IU/ml or IU/g or less, or of 5,000 ng/ml or ng/g or less;
IL-4 in a concentration of 1,000 ng/ml or ng/g or less;
BDNF in a concentration of 1,500 ng/ml or ng/g or less; and/or
IL-2 in a concentration of 100,000 IU/ml or IU/g less of IL-2, or of 6100 ng/ml or ng/g or less of IL-2.

25. Pharmaceutical composition (IV) comprising:
an IFN-γ-like acting substance;
an IL-4-like acting substance;
an BDNF-like acting substance; or
an IL-2-like acting substance, and
an immunomodulatory substance(s), wherein in case the pharmaceutical composition (IV) comprises:
the IFN-γ-like acting substance, the immunomodulatory substance(s) is different from the IFN-γ-like acting substance;
the IL-4-like acting substance, the immunomodulatory substance(s) is different from the IL-4-like acting substance;
the BDNF-like acting substance, the immunomodulatory substance(s) is different from the BDNF-like acting substance; and
the IL-2-like acting substance, the immunomodulatory substance(s) is different from the IL-2-like acting substance.

26. Injectable dosage form (I), comprising:
an IFN-γ-like acting substance;
an IL-4-like acting substance;
an BDNF-like acting substance; or
an IL-2-like acting substance, and
an immunomodulatory substance(s), wherein in case the injectable dosage form (I) comprises:
the IFN-γ-like acting substance, the immunomodulatory substance(s) is different from the IFN-γ-like acting substance;
the IL-4-like acting substance, the immunomodulatory substance(s) is different from the IL-4-like acting substance;
the BDNF-like acting substance, the immunomodulatory substance(s) is different from the BDNF-like acting substance; and
the IL-2-like acting substance, the immunomodulatory substance(s) is different from the IL-2-like acting substance.

27. Injectable dosage form (II), comprising:
an IFN-γ-like acting substance(s) in a total amount equivalent to 30,000 IU IFN-γ or less and/or in a total amount equivalent in activity to 1,500 ng IFN-γ or less;
an IL-4-like acting substance(s) in a total amount equivalent in activity to 1,000 ng IL-4 or less;
an BDNF-like acting substance(s) in a total amount equivalent in activity to 500 ng BDNF or less; or
an IL-2-like acting substance(s) a total amount equivalent to 1,000 IU IL-2 or less and/or in a total amount equivalent in activity to 61 ng IL-2 or less.

28. Injectable dosage form (II) according to claim 27, comprising:
IFN-γ in a total amount of 30,000 IU or less and/or in a total amount of 1,500 ng or less;
IL-4 in a total amount of 1,000 ng or less;
BDNF in a total amount of 500 ng or less; or
IL-2 a total amount of 1,000 IU or less and/or in a total amount of 61 ng or less.

29. Topical dosage form (I) comprising:
an IFN-γ-like acting substance;
an IL-4-like acting substance;
an BDNF-like acting substance; or
an IL-2-like acting substance.

30. Topical dosage form (II) comprising:
an immunomodulatory substance(s);
an energizing constituent.

31. Medical device (100) comprising:
a skin-conditioning unit (110); and
an applicator unit (120);
wherein the skin-conditioning unit (110) and the applicator unit (120) are configured to cooperatively interact to administer to a skin (102) of a subject an amount of a first therapeutic agent by means of the applicator unit (120) with an amount of conditioning measures by means of the skin-conditioning unit (110); and/or
wherein the skin-conditioning unit (110) and the applicator unit (120) are configured to act to a skin (102) of a subject.

32. Kit of parts (I) comprising:
i) an immunomodulatory substance(s); and
ii) a skin-conditioning agent; and/or
iii) an energizing means.

33. Kit of parts (II) comprising:
i) an IFN-γ-like acting substance, IL 4-like acting substance, BDNF-like acting substance and/or IL-2-like acting substance; and
ii) an immunomodulatory substance(s), wherein in case the Kit of parts (II) comprises:
the IFN-γ-like acting substance, the immunomodulatory substance(s) is different from the IFN-γ-like acting substance;
the IL-4-like acting substance, the immunomodulatory substance(s) is different from the IL-4-like acting substance;
the BDNF-like acting substance, the immunomodulatory substance(s) is different from the BDNF-like acting substance; and
the IL-2-like acting substance, the immunomodulatory substance(s) is different from the IL-2-like acting substance.

34. Kit of parts (III) comprising:
i) an injectable dosage form according to claim 26; and
ii) a skin-conditioning agent; and/or
iii) an energizing means.

35. Kit of parts (IV) comprising:
i) a topical dosage form according to claim 29; and
ii) a skin-conditioning agent; and/or
iv) an energizing means.

36. Kit of parts (V) comprising:
an applicator unit (120); and
a skin-conditioning unit (110); and
optionally a reservoir (121), comprising a first therapeutic agent like an immunomodulatory substance, wherein the reservoir (121) is configured to be fluidly coupled to the applicator unit (120).

37. The Pharmaceutical composition (I) according to claim 20;
further comprising a pharmaceutically acceptable vehicle, diluent, adjuvant, excipient, carrier, additive and/or salt.

38. Pharmaceutical composition (I) according to claim 20;
wherein the immunomodulatory substance(s) is a cytokine-like acting substance(s).

39. Pharmaceutical composition (II) according to claim 21;
wherein the IFN-γ-like acting substance is IFN-γ and/or any derivative, fragment, protein analogue, peptide, protein-variant, biopharmaceutical, inductor, precursor, prodrug, mutein, co-drug, propeptide thereof and/or any pharmaceutically acceptable salt of any of these,
the IL-4-like acting substance is IL-4 and/or any derivative, fragment, protein analogue, peptide, protein-variant, biopharmaceutical, inductor, precursor, prodrug, mutein, co-drug, propeptide thereof and/or any pharmaceutically acceptable salt of any of these,
the BDNF-like acting substance is BDNF and/or any derivative, fragment, protein analogue, peptide, protein-variant, biopharmaceutical, inductor, precursor, prodrug, mutein, co-drug, propeptide thereof and/or any pharmaceutically acceptable salt of any of these, and
the IL-2-like acting substance is IL-2 and/or any derivative, fragment, protein analogue, peptide, protein-variant, biopharmaceutical, inductor, precursor, prodrug, mutein, co-drug, propeptide thereof and/or any pharmaceutically acceptable salt of any of these.


SEQUENCE LISTING (4 sequences)

SEQ ID NO: 1 - protein, 399 aa, Homo sapiens
  REGION 1..399: note = huma IFN-gamma, human Interferon gamma 1b
METGLNASPPRTYRVALLYSGLALAGLASNLELYSLYSTYRPHEASNALAGLYHISSERASPVALALAASPASNGLYTHRLEPHELEGLYILELELYSASNTRPLYSGLGLSERASPARGLYSILEMETGLNSERGLNILEVALSERPHETYRPHELYSLEPHELYSASNPHELYSASPASPGLNSERILEGLNLYSSERVALGLTHRILELYSGLASPMETASNVALLYSPHEPHEASNSERASNLYSLYSLYSARGASPASPPHEGLLYSLETHRASNTYRSERVALTHRASPLEASNVALGLNARGLYSALAILEHISGLLEILEGLNVALMETALAGLLESERPRALAALALYSTHRGLYLYSARGLYSARGSERGLNMETLEPHEARGGLYARG

SEQ ID NO: 2 - protein, 132 aa, Homo sapiens
  REGION 1..132: note = human IL-2, human interleukin 2
PTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 3 - protein, 424 aa, Homo sapiens
  REGION 1..424: note = human IL-4, Interleukin 4
METGLYLETHRSERGLNLELEPRPRLEPHEPHELELEALACYSALAGLYASNPHEVALHISGLYHISLYSCYSASPILETHRLEGLNGLILEILELYSTHRLEASNSERLETHRGLGLNLYSTHRLECYSTHRGLLETHRVALTHRASPILEPHEALAALASERLYSASNTHRTHRGLLYSGLTHRPHECYSARGALAALATHRVALLEARGGLNPHETYRSERHISHISGLLYSASPTHRARGCYSLEGLYALATHRALAGLNGLNPHEHISARGHISLYSGLNLEILEARGPHELELYSARGLEASPARGASNLETRPGLYLEALAGLYLEASNSERCYSPRVALLYSGLALAASNGLNSERTHRLEGLASNPHELEGLARGLELYSTHRILEMETARGGLLYSTYRSERLYSCYSSERSER

SEQ ID NO: 4 - protein, 119 aa, Homo sapiens
  REGION 1..119: note = human BDNF, human brain-derived neurotrophic factor
HSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIKRGR